SEQUENCE LISTING (141 sequences)

SEQ ID NO: 1 - protein, 19 aa, Artificial Sequence
  Description of Artificial Sequence Synthetic peptide
KWASLWNWFNITNWLWYIK

SEQ ID NO: 2 - protein, 13 aa, Artificial Sequence
  Description of Artificial Sequence Synthetic peptide
KGWNWFDITNWGK

SEQ ID NO: 3 - protein, 13 aa, Artificial Sequence
  Description of Artificial Sequence Synthetic peptide
DKWXXXXXWFXIT

SEQ ID NO: 4 - protein, 17 aa, Artificial Sequence
  Description of Artificial Sequence Synthetic peptide
DKWXXXXXWFXITXXXW

SEQ ID NO: 5 - protein, 17 aa, Artificial Sequence
  Description of Artificial Sequence Synthetic peptide
DKWXXXXXWFXITXWXW

SEQ ID NO: 6 - protein, 26 aa, Artificial Sequence
  Description of Artificial Sequence Synthetic peptide
XXXXXXXXNWFXITXWLWXXXXXXXX

SEQ ID NO: 7 - protein, 14 aa, Artificial Sequence
  Description of Artificial Sequence Synthetic peptide
NWFCITXWLWKKKK

SEQ ID NO: 8 - protein, 16 aa, Artificial Sequence
  Description of Artificial Sequence Synthetic peptide
NWFDITNWLWYIKKKK

SEQ ID NO: 9 - protein, 15 aa, Artificial Sequence
  Description of Artificial Sequence Synthetic peptide
NWFDITNWLWKXKXK

SEQ ID NO: 10 - protein, 18 aa, Artificial Sequence
  Description of Artificial Sequence Synthetic peptide
KKXNWFDITNWLWKXKXK

SEQ ID NO: 11 - protein, 18 aa, Artificial Sequence
  Description of Artificial Sequence Synthetic peptide
NWFDITNWLWYIKXKXKK

SEQ ID NO: 12 - protein, 14 aa, Artificial Sequence
  Description of Artificial Sequence Synthetic peptide
NWFCITXWLWKKKK

SEQ ID NO: 13 - protein, 18 aa, Artificial Sequence
  Description of Artificial Sequence Synthetic peptide
NWFXITXWLWXXXXXXXX

SEQ ID NO: 14 - protein, 13 aa, Artificial Sequence
  Description of Artificial Sequence Synthetic peptide
SLWNWFDITNWLW

SEQ ID NO: 15 - protein, 14 aa, Artificial Sequence
  Description of Artificial Sequence Synthetic peptide
NWFDITNWLWKKKK

SEQ ID NO: 16 - protein, 19 aa, Artificial Sequence
  Description of Artificial Sequence Synthetic peptide
SLWNWFDITNWLWYIKKKK

SEQ ID NO: 17 - protein, 12 aa, Artificial Sequence
  Description of Artificial Sequence Synthetic peptide
RCRTIDVFRNCI

SEQ ID NO: 18 - protein, 15 aa, Artificial Sequence
  Description of Artificial Sequence Synthetic peptide
AEPAETSWFYLTTFL

SEQ ID NO: 19 - protein, 12 aa, Artificial Sequence
  Description of Artificial Sequence Synthetic peptide
NWFDITNWLWRR

SEQ ID NO: 20 - protein, 15 aa, Artificial Sequence
  Description of Artificial Sequence Synthetic peptide
SLWNWFDITNWLWRR

SEQ ID NO: 21 - protein, 19 aa, Artificial Sequence
  Description of Artificial Sequence Synthetic peptide
DKWASLWNWFDITNWLWRR

SEQ ID NO: 22 - protein, 15 aa, Artificial Sequence
  Description of Artificial Sequence Synthetic peptide
WNWFDITNWLWKKKK

SEQ ID NO: 23 - protein, 16 aa, Artificial Sequence
  Description of Artificial Sequence Synthetic peptide
LWNWFDITNWLWKKKK

SEQ ID NO: 24 - protein, 17 aa, Artificial Sequence
  Description of Artificial Sequence Synthetic peptide
SLWNWFDITNWLWKKKK

SEQ ID NO: 25 - protein, 14 aa, Artificial Sequence
  Description of Artificial Sequence Synthetic peptide
NWFDITNWLAKKKK

SEQ ID NO: 26 - protein, 15 aa, Artificial Sequence
  Description of Artificial Sequence Synthetic peptide
WNWFDITNWLAKKKK

SEQ ID NO: 27 - protein, 16 aa, Artificial Sequence
  Description of Artificial Sequence Synthetic peptide
LWNWFDITNWLAKKKK

SEQ ID NO: 28 - protein, 17 aa, Artificial Sequence
  Description of Artificial Sequence Synthetic peptide
SLWNWFDITNWLAKKKK

SEQ ID NO: 29 - protein, 6 aa, Artificial Sequence
  Description of Artificial Sequence Synthetic peptide
WFDITX

SEQ ID NO: 30 - protein, 7 aa, Artificial Sequence
  Description of Artificial Sequence Synthetic peptide
NWFDITX

SEQ ID NO: 31 - protein, 8 aa, Artificial Sequence
  Description of Artificial Sequence Synthetic peptide
XNWFDITX

SEQ ID NO: 32 - protein, 14 aa, Artificial Sequence
  Description of Artificial Sequence Synthetic peptide
DKWASLXNWFDITX

SEQ ID NO: 33 - protein, 16 aa, Artificial Sequence
  Description of Artificial Sequence Synthetic peptide
ELDKWASLXNWFDITX

SEQ ID NO: 34 - protein, 12 aa, Artificial Sequence
  Description of Artificial Sequence Synthetic peptide
NWFDITNXLWRR

SEQ ID NO: 35 - protein, 15 aa, Artificial Sequence
  Description of Artificial Sequence Synthetic peptide
SLXNWFDITNXLWRR

SEQ ID NO: 36 - protein, 19 aa, Artificial Sequence
  Description of Artificial Sequence Synthetic peptide
DKWXSLXNWFDITNXLWRR

SEQ ID NO: 37 - protein, 7 aa, Artificial Sequence
  Description of Artificial Sequence Synthetic peptide
CAWFXIT

SEQ ID NO: 38 - protein, 7 aa, Artificial Sequence
  Description of Artificial Sequence Synthetic peptide
CAWFXIT

SEQ ID NO: 39 - protein, 7 aa, Artificial Sequence
  Description of Artificial Sequence Synthetic peptide
CAWFXIT

SEQ ID NO: 40 - protein, 7 aa, Artificial Sequence
  Description of Artificial Sequence Synthetic peptide
CAWFXIT

SEQ ID NO: 41 - protein, 9 aa, Artificial Sequence
  Description of Artificial Sequence Synthetic peptide
KKCAWFXIT

SEQ ID NO: 42 - protein, 9 aa, Artificial Sequence
  Description of Artificial Sequence Synthetic peptide
KKCAWFXIT

SEQ ID NO: 43 - protein, 13 aa, Artificial Sequence
  Description of Artificial Sequence Synthetic peptide
CNWFXITNWLWRR

SEQ ID NO: 44 - protein, 13 aa, Artificial Sequence
  Description of Artificial Sequence Synthetic peptide
CNWFXITNWLWRR

SEQ ID NO: 45 - protein, 19 aa, Artificial Sequence
  Description of Artificial Sequence Synthetic peptide
DKWASLCNWFXITNWLWRR

SEQ ID NO: 46 - protein, 19 aa, Artificial Sequence
  Description of Artificial Sequence Synthetic peptide
DKWASLCNWFXITNWLWRR

SEQ ID NO: 47 - protein, 22 aa, Artificial Sequence
  Description of Artificial Sequence Synthetic peptide
LELDKWASLCNWFXITNWLWRR

SEQ ID NO: 48 - protein, 22 aa, Artificial Sequence
  Description of Artificial Sequence Synthetic peptide
LELDKWASLCNWFXITNWLWRR

SEQ ID NO: 49 - protein, 12 aa, Artificial Sequence
  Description of Artificial Sequence Synthetic peptide
CWFXITNWLWKK

SEQ ID NO: 50 - protein, 12 aa, Artificial Sequence
  Description of Artificial Sequence Synthetic peptide
CWFXITNWLWKK

SEQ ID NO: 51 - protein, 13 aa, Artificial Sequence
  Description of Artificial Sequence Synthetic peptide
WCWFXITNWLWKK

SEQ ID NO: 52 - protein, 14 aa, Artificial Sequence
  Description of Artificial Sequence Synthetic peptide
CWFXITNWLWKKKK

SEQ ID NO: 53 - protein, 14 aa, Artificial Sequence
  Description of Artificial Sequence Synthetic peptide
CWFXITNWLWKKKK

SEQ ID NO: 54 - protein, 15 aa, Artificial Sequence
  Description of Artificial Sequence Synthetic peptide
WCWFXITNWLWKKKK

SEQ ID NO: 55 - protein, 15 aa, Artificial Sequence
  Description of Artificial Sequence Synthetic peptide
WCWFXITNWLWKKKK

SEQ ID NO: 56 - protein, 16 aa, Artificial Sequence
  Description of Artificial Sequence Synthetic peptide
NWFEITNKLWGRRRRC

SEQ ID NO: 57 - protein, 16 aa, Artificial Sequence
  Description of Artificial Sequence Synthetic peptide
NWFEITNKLWGRRRRC

SEQ ID NO: 58 - protein, 18 aa, Artificial Sequence
  Description of Artificial Sequence Synthetic peptide
LWNWFEITNKLWGRRRRC

SEQ ID NO: 59 - protein, 18 aa, Artificial Sequence
  Description of Artificial Sequence Synthetic peptide
LWNWFEITNKLWGRRRRC

SEQ ID NO: 60 - protein, 23 aa, Artificial Sequence
  Description of Artificial Sequence Synthetic peptide
DKWASLWNWFEITNKLWGRRRRC

SEQ ID NO: 61 - protein, 23 aa, Artificial Sequence
  Description of Artificial Sequence Synthetic peptide
DKWASLWNWFEITNKLWGRRRRC

SEQ ID NO: 62 - protein, 27 aa, Artificial Sequence
  Description of Artificial Sequence Synthetic peptide
LLELDKWASLWNWFEITNKLWGRRRRC

SEQ ID NO: 63 - protein, 27 aa, Artificial Sequence
  Description of Artificial Sequence Synthetic peptide
LLELDKWASLWNWFEITNKLWGRRRRC

SEQ ID NO: 64 - protein, 16 aa, Artificial Sequence
  Description of Artificial Sequence Synthetic peptide
NWFEITNWLWGRRRRC

SEQ ID NO: 65 - protein, 23 aa, Artificial Sequence
  Description of Artificial Sequence Synthetic peptide
DKWASLKNWFEITNWLWGRRRRC

SEQ ID NO: 66 - protein, 23 aa, Artificial Sequence
  Description of Artificial Sequence Synthetic peptide
DKWASLKNWFEITNWLWGRRRRC

SEQ ID NO: 67 - protein, 27 aa, Artificial Sequence
  Description of Artificial Sequence Synthetic peptide
LLELDKWASLKNWFEITNWLWGRRRRC

SEQ ID NO: 68 - protein, 27 aa, Artificial Sequence
  Description of Artificial Sequence Synthetic peptide
LLELDKWASLKNWFEITNWLWGRRRRC

SEQ ID NO: 69 - protein, 14 aa, Artificial Sequence
  Description of Artificial Sequence Synthetic peptide
EWFKITNWLWKKKK

SEQ ID NO: 70 - protein, 14 aa, Artificial Sequence
  Description of Artificial Sequence Synthetic peptide
EWFKITNWLWKKKK

SEQ ID NO: 71 - protein, 15 aa, Artificial Sequence
  Description of Artificial Sequence Synthetic peptide
WEWFKITNWLWKKKK

SEQ ID NO: 72 - protein, 15 aa, Artificial Sequence
  Description of Artificial Sequence Synthetic peptide
WEWFKITNWLWKKKK

SEQ ID NO: 73 - protein, 13 aa, Artificial Sequence
  Description of Artificial Sequence Synthetic peptide
NWFDITNWLWYIK

SEQ ID NO: 74 - protein, 15 aa, Artificial Sequence
  Description of Artificial Sequence Synthetic peptide
WNWFDITNWLWKKKK

SEQ ID NO: 75 - protein, 18 aa, Artificial Sequence
  Description of Artificial Sequence Synthetic peptide
NWFDITNWLWYIKXKXKK

SEQ ID NO: 76 - protein, 6 aa, Artificial Sequence
  Description of Artificial Sequence Synthetic peptide
ELDKWA

SEQ ID NO: 77 - protein, 6 aa, Artificial Sequence
  Description of Artificial Sequence Synthetic peptide
NWFXIT

SEQ ID NO: 78 - protein, 5 aa, Artificial Sequence
  Description of Artificial Sequence Synthetic peptide
WFXIT

SEQ ID NO: 79 - protein, 5 aa, Artificial Sequence
  Description of Artificial Sequence Synthetic peptide
WFXIT

SEQ ID NO: 80 - protein, 11 aa, Artificial Sequence
  Description of Artificial Sequence Synthetic peptide
LWNWFDISQWL

SEQ ID NO: 81 - protein, 8 aa, Artificial Sequence
  Description of Artificial Sequence Synthetic peptide
LLELDKWA

SEQ ID NO: 82 - protein, 7 aa, Artificial Sequence
  Description of Artificial Sequence Synthetic peptide
ELDKWAS

SEQ ID NO: 83 - protein, 11 aa, Artificial Sequence
  Description of Artificial Sequence Synthetic peptide
LWNWFDITNWL

SEQ ID NO: 84 - protein, 6 aa, Artificial Sequence
  Description of Artificial Sequence Synthetic peptide
SGGRGG

SEQ ID NO: 85 - protein, 6 aa, Artificial Sequence
  Description of Artificial Sequence Synthetic peptide
NWFDIT

SEQ ID NO: 86 - protein, 6 aa, Artificial Sequence
  Description of Artificial Sequence Synthetic peptide
NWFNIT

SEQ ID NO: 87 - protein, 10 aa, Artificial Sequence
  Description of Artificial Sequence Synthetic peptide
NWFDITNWLW

SEQ ID NO: 88 - protein, 16 aa, Artificial Sequence
  Description of Artificial Sequence Synthetic peptide
SLWNWFDITNWLWRRK

SEQ ID NO: 89 - protein, 14 aa, Artificial Sequence
  Description of Artificial Sequence Synthetic peptide
NWFCITXWLWKKKK

SEQ ID NO: 90 - protein, 12 aa, Artificial Sequence
  Description of Artificial Sequence Synthetic peptide
NWFDITNXLWRR

SEQ ID NO: 91 - protein, 12 aa, Artificial Sequence
  Description of Artificial Sequence Synthetic peptide
GVIPLLTITNYA

SEQ ID NO: 92 - protein, 18 aa, Artificial Sequence
  Description of Artificial Sequence Synthetic peptide
EGTTGWGWLGKPIGAFAH

SEQ ID NO: 93 - protein, 17 aa, Artificial Sequence
  Description of Artificial Sequence Synthetic peptide
ELDKWANLWNWFDITNW

SEQ ID NO: 94 - protein, 24 aa, Artificial Sequence
  Description of Artificial Sequence Synthetic peptide
LLELDKWASLWNWFNITNWLWYIK

SEQ ID NO: 95 - protein, 11 aa, Artificial Sequence
  Description of Artificial Sequence Synthetic peptide
LWDWFNISNWL

SEQ ID NO: 96 - protein, 11 aa, Artificial Sequence
  Description of Artificial Sequence Synthetic peptide
LWSWFDITNWL

SEQ ID NO: 97 - protein, 11 aa, Artificial Sequence
  Description of Artificial Sequence Synthetic peptide
LWNWFNITNWL

SEQ ID NO: 98 - protein, 11 aa, Artificial Sequence
  Description of Artificial Sequence Synthetic peptide
LWNWFDITKWL

SEQ ID NO: 99 - protein, 11 aa, Artificial Sequence
  Description of Artificial Sequence Synthetic peptide
LWSWFSITNWL

SEQ ID NO: 100 - protein, 11 aa, Artificial Sequence
  Description of Artificial Sequence Synthetic peptide
LWNWFXITNWL

SEQ ID NO: 101 - protein, 11 aa, Artificial Sequence
  Description of Artificial Sequence Synthetic peptide
LWNWFDISKWL

SEQ ID NO: 102 - protein, 11 aa, Artificial Sequence
  Description of Artificial Sequence Synthetic peptide
LWNWFNITNWL

SEQ ID NO: 103 - protein, 11 aa, Artificial Sequence
  Description of Artificial Sequence Synthetic peptide
LWXWFDITQWL

SEQ ID NO: 104 - protein, 11 aa, Artificial Sequence
  Description of Artificial Sequence Synthetic peptide
LWNWFDISXWL

SEQ ID NO: 105 - protein, 11 aa, Artificial Sequence
  Description of Artificial Sequence Synthetic peptide
LWXWFDISNWL

SEQ ID NO: 106 - protein, 11 aa, Artificial Sequence
  Description of Artificial Sequence Synthetic peptide
LWSWFNISNWL

SEQ ID NO: 107 - protein, 11 aa, Artificial Sequence
  Description of Artificial Sequence Synthetic peptide
LWNWFNITKWL

SEQ ID NO: 108 - protein, 11 aa, Artificial Sequence
  Description of Artificial Sequence Synthetic peptide
LWNWFXISXWL

SEQ ID NO: 109 - protein, 11 aa, Artificial Sequence
  Description of Artificial Sequence Synthetic peptide
LWNWFSITKWL

SEQ ID NO: 110 - protein, 11 aa, Artificial Sequence
  Description of Artificial Sequence Synthetic peptide
LWSWFDISKWL

SEQ ID NO: 111 - protein, 11 aa, Artificial Sequence
  Description of Artificial Sequence Synthetic peptide
LWNWFSITNWL

SEQ ID NO: 112 - protein, 11 aa, Artificial Sequence
  Description of Artificial Sequence Synthetic peptide
LWTWFDITNWL

SEQ ID NO: 113 - protein, 11 aa, Artificial Sequence
  Description of Artificial Sequence Synthetic peptide
LWSWFDITKWL

SEQ ID NO: 114 - protein, 11 aa, Artificial Sequence
  Description of Artificial Sequence Synthetic peptide
LWSWFNISNWL

SEQ ID NO: 115 - protein, 11 aa, Artificial Sequence
  Description of Artificial Sequence Synthetic peptide
LWNWFDISRWL

SEQ ID NO: 116 - protein, 11 aa, Artificial Sequence
  Description of Artificial Sequence Synthetic peptide
LWNWFNITEWL

SEQ ID NO: 117 - protein, 11 aa, Artificial Sequence
  Description of Artificial Sequence Synthetic peptide
LWNWFDIXXWL

SEQ ID NO: 118 - protein, 11 aa, Artificial Sequence
  Description of Artificial Sequence Synthetic peptide
LWNWFNITNWL

SEQ ID NO: 119 - protein, 11 aa, Artificial Sequence
  Description of Artificial Sequence Synthetic peptide
LWXWFDIXNWL

SEQ ID NO: 120 - protein, 11 aa, Artificial Sequence
  Description of Artificial Sequence Synthetic peptide
LWNWFDISNWL

SEQ ID NO: 121 - protein, 11 aa, Artificial Sequence
  Description of Artificial Sequence Synthetic peptide
LWNWFGITKWL

SEQ ID NO: 122 - protein, 11 aa, Artificial Sequence
  Description of Artificial Sequence Synthetic peptide
LWNWFDISNWL

SEQ ID NO: 123 - protein, 11 aa, Artificial Sequence
  Description of Artificial Sequence Synthetic peptide
LWNWFDITXWL

SEQ ID NO: 124 - protein, 11 aa, Artificial Sequence
  Description of Artificial Sequence Synthetic peptide
LWXWFSIXNWL

SEQ ID NO: 125 - protein, 11 aa, Artificial Sequence
  Description of Artificial Sequence Synthetic peptide
LWNWFSITKWL

SEQ ID NO: 126 - protein, 11 aa, Artificial Sequence
  Description of Artificial Sequence Synthetic peptide
LWNWFEITNWL

SEQ ID NO: 127 - protein, 11 aa, Artificial Sequence
  Description of Artificial Sequence Synthetic peptide
LWNWFTISKWL

SEQ ID NO: 128 - protein, 11 aa, Artificial Sequence
  Description of Artificial Sequence Synthetic peptide
LWNWFSISKWL

SEQ ID NO: 129 - protein, 11 aa, Artificial Sequence
  Description of Artificial Sequence Synthetic peptide
LWNWFDITQWL

SEQ ID NO: 130 - protein, 11 aa, Artificial Sequence
  Description of Artificial Sequence Synthetic peptide
LWTWFDISNWL

SEQ ID NO: 131 - protein, 11 aa, Artificial Sequence
  Description of Artificial Sequence Synthetic peptide
LWNWFDISNWL

SEQ ID NO: 132 - protein, 11 aa, Artificial Sequence
  Description of Artificial Sequence Synthetic peptide
LWNWFSISXWL

SEQ ID NO: 133 - protein, 11 aa, Artificial Sequence
  Description of Artificial Sequence Synthetic peptide
LWXWFXISNWL

SEQ ID NO: 134 - protein, 11 aa, Artificial Sequence
  Description of Artificial Sequence Synthetic peptide
LWNWFSITQWL

SEQ ID NO: 135 - protein, 11 aa, Artificial Sequence
  Description of Artificial Sequence Synthetic peptide
LWNWFSITQWL

SEQ ID NO: 136 - protein, 11 aa, Artificial Sequence
  Description of Artificial Sequence Synthetic peptide
LWNWFGISXWL

SEQ ID NO: 137 - protein, 11 aa, Artificial Sequence
  Description of Artificial Sequence Synthetic peptide
LWTWFDITKWL

SEQ ID NO: 138 - protein, 11 aa, Artificial Sequence
  Description of Artificial Sequence Synthetic peptide
LWXWFDISXWL

SEQ ID NO: 139 - protein, 11 aa, Artificial Sequence
  Description of Artificial Sequence Synthetic peptide
LWSWFXISNWL

SEQ ID NO: 140 - protein, 11 aa, Artificial Sequence
  Description of Artificial Sequence Synthetic peptide
LWNWFSITKWL

SEQ ID NO: 141 - protein, 11 aa, Artificial Sequence
  Description of Artificial Sequence Synthetic peptide
LWNWFTITKWL

(19) United States
(12) Patent Application Publication
(10) Pub. No.: US 2011/0124842 A1
(43) Pub. Date: May 26, 2011

(54) Peptide that binds to a broadly neutralizing anti-HIV antibody-structure of 4E10 Fab fragment complex, uses thereof, compositions therefrom

(76) Inventors: Florence M. Brunel, La Jolla, CA (US); Dennis R. Burton, La Jolla, CA (US); Rosa M.F. Cardoso, Del Mar, CA (US); Philip E. Dawson, San Diego, CA (US); Sharon Ferguson, La Jolla, CA (US); Ian A. Wilson, La Jolla, CA (US); Michael B. Zwick, San Diego, CA (US)

(21) Appl. No.: 11/489,162

(22) Filed: Jul. 19, 2006

Related U.S. Application Data
(63) Continuation-in-part of application No. 10/946,371, filed on Sep. 20, 2004.
(60) Provisional application No. 60/504,123, filed on Sep. 19, 2003.

(51) Int. Cl. C07K 7/08; C07K 7/06

(52) U.S. Cl. 530/326; 530/327

(57) ABSTRACT

The present invention relates to the structure of Fab 4E10, e.g., as a complex with herein identified peptide KGND, herein identified as a 4E10 mimetope on gp41, as determined by crystallographic techniques, and the confirmation that peptide KGND has a functional relevant conformation, as well as the determination of key residues on 4E10, and uses thereof and compounds and compositions therefrom. Furthermore, the invention also relates to other peptides and mimetic peptides which bind to Fab 4E10.

### CROSS-REFERENCE TO RELATED APPLICATIONS

This application is a continuation-in-part of U.S. patent application Ser. No. 10/946,371 filed Sep. 20, 2004, which claims priority to U.S. Provisional Patent Application Ser. No. 60/504,123, filed on Sep. 19, 2003. This application also makes reference to various documents cited in this text, including International Application Patent Application PCT/EP00/10070, filed Sep. 9, 2002, and published on Mar. 20, 2003 as WO 03/022879.

Citations in the text can be by way of citation to a document in the reference list or by full citation in the text to a document that may or may not also be listed in the reference list. There is no admission that any of the various documents cited in this text are prior art as to the present invention. Any document having as an author or inventor person or persons named as an inventor herein is a document that is not by another as to the inventive entity herein. All documents cited in this text (“herein cited document”) and all documents cited or referenced in herein cited documents are hereby incorporated by reference, including the text, figures, and sequence listing of WO 03/022879. Likewise, teachings of herein cited documents and documents cited in herein cited documents can be employed in the practice and utilities of the present invention.

### GOVERNMENT SUPPORT/OTHER GRANTS

The development of inventions herein was supported by grants from the National Institutes of Health (NIH) Grant Nos. A1058725, A133292, GM46192 and MH0622961. Also, funding for developments of inventions herein was provided by the International AIDS Vaccine Initiative (IAVI), No. SFP-1442 and the American Foundation for AIDS Research. The United States government, IAVI and the American Foundation for AIDS Research may have certain rights to the present invention.

### FIELD OF THE INVENTION

The invention relates to the structure of Fab 4E10, e.g., as a complex with herein identified peptide KGND, herein identified as a 4E10 mimetope on gp41, as determined by crystallographic techniques, and to the confirmation that peptide KGND has a functional relevant conformation, as well as to the determination of key residues on 4E10. The present invention thus provides a means for identifying or designing compounds, such as, but not limited to, peptides or derivatized peptides (e.g., N-acylated or N-alkylated peptides), that bind to the antibody. These compounds, when administered, elicit anti-HIV antibodies. The compounds may then be used in diagnostic, pharmaceutical, immunogenic, immunological or vaccine compositions. These compositions are useful in the detection or treatment and/or prevention of HIV infections, specifically Glade B infections, although variants may be effective against any one or more of clades A, C, D, or E. Further, antibodies elicited by such compounds also can be used in diagnostic or pharmaceutical, immunogenic, immunological or vaccine compositions. The invention also relates to the use of the structure of KGND, e.g., as determined by crystallographic techniques to identify further compounds or antibodies, which would bind to KGND, which compounds or antibodies are useful in diagnostic, pharmaceutical, immunogenic, immunological compositions, e.g., as such compounds or antibodies bind to HIV immunogens, antigens or epitopes.

The invention also relates to data storage media encoded with the structural data, e.g., coordinates of crystallized 4E10 or at least a functional portion thereof and/or KGND. Such data storage material is capable of displaying such structures, or their structural homologues, as a graphical three-dimensional representation on a computer screen. This invention also relates to methods of using the structure co-ordinates to solve the structure of compounds that similarly complex with 4E10, as well as compounds that complex with KGND. In addition, this invention relates to methods of using structure co-ordinates to screen and design compounds that bind to 4E10, as well as compounds that bind to KGND. The invention further relates to transmission of information concerning such compounds.

Other aspects of the invention are discussed in or are obvious from the text of this document.

### BACKGROUND OF THE INVENTION

The development of a vaccine is considered to be the best hope for controlling the acquired immune deficiency syndrome (AIDS) epidemic. A vaccine should elicit two components: neutralizing antibodies and cytotoxic T lymphocytes, CTL. This can be achieved by immunization with dead virus or immunogenic peptides or proteins from the infectious agent. However, in the case of human immunodeficiency virus (HIV), these approaches have not yet been successful. Protection against both intravenous and vaginal simian-human immunodeficiency virus (SHIV) challenges by neutralizing antibodies has been shown in macaques (Parren, 2001; Mascola, 2000; Shibata, 1999).

In addition, an effective vaccine should elicit a broadly neutralizing antibody response, since a wide variety of strains of the virus exist. Broadly neutralizing antibodies recognize exposed conserved regions on gp120 and gp41 on envelope spikes on the surface of the virus. Their existence was demonstrated by the activity of certain HIV sera; and broadly neutralizing antibodies have been described (Burton, 1994; Conley, 1994; Burton, 1996; Zwick, 2001).

The HIV type 1 (HIV-1) transmembrane glycoprotein gp41 mediates viral fusion with host cells (Chan, 1998). Before fusion, gp41 exists as a trimeric complex associated with gp120, and has limited accessibility. The broadly neutralizing human monoclonal antibodies 2F5 and 4E10 appear to recognize structures that are present to some degree even after binding of virus to the target cell (Binley, 2003). Their epitopes are close and are found in a region of gp41 proximal to the membrane (see FIG. 42). FIG. 42A provides the structure of gp41, and FIG. 42B depicts the current model wherein HIV gp41 undergoes major structural arrangements.

The native state of the 120-gp41 complex is metastable and triggered by gp120 binding to CD4 and coreceptor (here CCR5). The 4E10 epitope on gp41 is represented as a pink helix parallel to the plane of the viral membrane and the epitope seems to be exposed and susceptible to antibody binding and virus neutralization in the metastable and receptor-bound states of gp41. Conformational changes of the Env proteins leading to the pre-hairpin intermediate cause gp120 dissociation of gp41 and insertion of the gp41 fusion peptide into the host cell membrane. For clarity, only one gp41 monomer is shown for the pre-hairpin state (N-terminal heptad repeat is a pink helix and C-terminal heptad repeat is a green helix). 4E10 binding to the extended pre-hairpin intermediate is a possibility to be still proved. The viral and cell membranes are brought into close proximity and the orientation of the helical gp41 membrane-proximal region parallel to the membranes with the Trp residues around the helical axis could aid in the disruption of both membranes. In the final stages of fusion, the C-terminal heptad repeat folds back onto the N-terminal heptad repeat to generate a trimer of hairpins also known as the 6-helix bundle structure.

Different routes have been explored to elicit broadly neutralizing antibodies. One of them consists of trying to generate immunogens that will induce a 2F5-like immune response. However, immunizations with peptides containing the 2F5 sequence have failed to elicit neutralizing antibodies, possibly because these peptides do not adopt the same conformation as gp41 during fusion. As a result, antibodies bind to the peptide epitope but do not neutralize.

Only a handful of potent and broadly cross-reactive human monoclonal antibodies (MAbs) have being identified to date against HIV-1 primary isolates and include MAbs b12, 2G12, 2F5, and 4E10. These rare MAbs have been derived from HIV-1 infected patients and target conserved, but distinct, epitopes on gp120 or gp41, the HIV-1 envelope (Env) glycoproteins responsible for mediating HIV entry into human cells (Weissenhorn et al., 1997; Chan et al., 1997; Kwong et al., 1998; Wyatt and Sodroski, 1998). MAb b12 binds to the recessed CD4 binding site on gp120 (Saphire et al., 2001), whereas MAb 2G12 recognizes a unique cluster of oligomannose sugars on the gp120 outer domain (Calarese et al., 2003). MAbs 4E10 and 2F5 both recognize adjacent and conserved contiguous epitopes in the C-terminal membrane-proximal region of gp41 (FIG. 37A), indicating that gp41 is not completely masked by gp120 from Ab recognition. The 2F5 epitope is centered around the sequence ELDKWA (SEQ ID NO: 76) (Muster et al., 1993; Zwick et al., 2001a; Barbato et al., 2003), whereas 4E10 recognizes an epitope containing the sequence NWF(D/N)IT (SEQ ID NO: 77) (Zwick et al., 2001b) in a Trp-rich region of gp41 immediately C-terminal to the 2F5 epitope.

Other reports that have identified neutralizing antibodies (such as 2F5 and 4E10) against human immunodeficiency virus glycoproteins, such as gp41 include, for instance, Stiegler et al., 2001; Ferrantelli et al., 2003; Ktabwalla et al., 2003; Ruprecht et al., 2003. Mention is also made of Schibli et al., 2001 that relates to the NMR structure of a peptide that shows a helical structure. Mention is also made of Barbato, G. et al. 2003, McGaughey, G. B., 2003, Biron, Z. et al., 2002, and Joyce, J. G. et al., 2002; which show there is controversy in the art as to the structure of peptides, such as gp41 and portions thereof.

Studies have been done to elucidate the crystal structure of biologically significant proteins and modulators thereof, such as cytochrome P450 2C9, Beta-Site APP Cleaving Enzyme, ketopantoate reductase, ketopantoate hydroxymethyl transferase, pantothenate synetase; see, e.g., PCT Patent Application Publication Nos. WO 02/077270, WO 03/035693, WO 03/012089, WO 02/095035, WO 02/079490, WO 02/0222793.

It would thus be desirable to identify the structure of Fab 4E10, e.g., in complex with a herein identified peptide KGND, herein identified as a 4E10 mimetope on gp41, such as by way of crystallographic techniques, and confirm that peptide KGND has a functional relevant conformation. These techniques would also provide a determination of key residues on 4E10, to provide means for identifying or designing compounds, such as peptides or derivatized peptides (e.g., N-acylated or N-alkylated peptides), that bind to the antibody, and thus when administered elicit anti-HIV antibodies; the compounds may then be used in diagnostic, pharmaceutical, immunogenic, immunological or vaccine compositions, useful in the detection or treatment and/or prevention of HIV infections, and which antibodies can be used in diagnostic or pharmaceutical, immunogenic, immunological or vaccine compositions. Such compounds may also be made on synthetic backbones or scaffolds which would provide the correct spacing and distribution for the side chains.

In addition, the study of crystal structure and symmetry is developed (See, e.g., Cotton and Wilkinson, Inorganic Chemistry (John Wiley & Sons, Fourth Ed. 1980), especially Ch. 2). X-ray crystallography, or more generally crystallography, is an established, well-studied technique that provides what can best be described as a three-dimensional picture of what a molecule looks like in a crystal, and is useful for determining whether a compound that is not a known ligand of a target biomolecule can indeed bind as a ligand to a target biomolecule (see, e.g., WO 99/45379; U.S. Pat. No. 6,087,478; U.S. Pat. No. 6,110,672); and, there are additional techniques for identifying drug cores (see, e.g., WO 98/57155 regarding fragment-based screening). Mention is also made of U.S. Pat. Nos. 6,128,582, 6,153,579, 6,077,682, and 6,037,117 and PCT publications WO01/37194 and WO00/47763 for additional information on aspects of structure-based drug design and homology modelling.

These techniques can be employed with the herein disclosed 4E10 crystals and proteins, to rationally design compounds that bind to or interact with 4E10; and, the use of these techniques, in combination with herein disclosed 4E10 crystals and proteins it is believed has not been heretofore taught or suggested in the art.

As previously stated, simultaneous targeting of multiple conserved epitopes on HIV appears to be the best strategy for vaccine development to maximize the breadth of protection (Zwick et al., 2001b; Kitabwalla et al., 2003). As a single agent, 4E10 is the broadest neutralizing MAb described to date with activity against most isolates from HIV-1 clades, including A, B, C, D, E, and G, albeit sometimes with less potency compared to the other three more restricted MAbs described above. The breadth and potency of 4E10 was recently evaluated against a panel of 93 viruses in a pseudovirus assay (Binley et al. Manuscript in preparation). From this extensive analysis, 4E10 neutralizes viruses with a variety of substitutions in the NWF(D/N)IT (SEQ ID NO: 77) motif comprising the 4E10 epitope (FIG. 37B). The minimal epitope for 4E10 from this study was defined as WFXI, where X can be D, N, S, G, E, or T. However, several HIV isolates with the same 4E10 target epitope are differentially neutralized with orders of magnitude difference in potencies (Binley et al. Manuscript in preparation), implying that the 4E10 epitope is not constitutively exposed on all viruses, but differences in Env conformation or different infection kinetics might influence accessibility to the 4E10 epitope.

Broadly neutralizing monoclonal antibodies to HIV-1 like 4E10 are invaluable tools for vaccine design and the description of the binding of 4E10 to its peptide epitope should assist in the design of immunogens able of eliciting 4E10-like neutralizing responses. The fact that the 4E10 epitope is contiguous and has a biologically-relevant helical conformation, makes the epitope a very good lead for structure-based design of a broadly effective HIV-1 vaccine. The importance of understanding why only a few antibodies can neutralize primary isolates of HIV-1 is of fundamental importance for the design of an HIV-1 vaccine and for generating a broad immune response that would be effective against the multiple isolates and clades of HIV-1 found worldwide.

The conserved C-terminal region of the 41 extracellular domain that encompasses the 4E10 and 2F5 epitopes is critical for Env-mediated membrane fusion and virus infectivity (Salzwedel et al., 1999; Munoz-Barroso et al., 1999). Alanine mutation of three of five conserved tryptophan residues (Trp⁶⁶⁶, Trp⁶⁷⁰, and Trp⁶⁷²; numbered according to the HXB2 isolate sequence) in this membrane-proximal gp41 region abolishes viral entry (Salzwedel et al., 1999). Moreover, the induction of membrane leakage by a peptide corresponding to this Trp-rich region (Suarez et al., 2000) implies that this region may be directly involved in membrane disruption during the fusion process. However, this notion has been challenged by another mutagenesis study which suggests that the membrane-proximal region instead provides a flexible arm to gp41 to allow membrane fusion (Dimitrov et al., 2003). Overall, the conserved membrane-proximal region of gp41 appears to be highly promising for vaccine development, especially since it is the target of two (4E10 and 2F5) of the four most broadly neutralizing HIV MAbs.

The three-dimensional structure of the Trp-rich membrane-proximal region of gp41 was previously investigated by NMR spectroscopy using a synthetic peptide (KWASLWNWFNITNWLWYIK) (SEQ ID NO: 1; Schibli et al., 2001). In dodecylphosphocholine micelles, the Trp-rich region has a helical structure with the Trp residues forming a “collar” around the helix axis, parallel to the water-dodecylphosphocholine interface of the micelle. However, the precise orientation of this region in the natural context of the native gp120-gp41 trimer and how it might rearrange during the fusion process remain unknown. To examine the interaction of 4E10 with its epitope on gp41 at the atomic level, we determined the crystal structure of Fab 4E10 in complex with a soluble synthetic 13-residue peptide (KGWNWFDITNWGK) (SEQ ID NO: 2; Zwick et al., 2001a) that encompasses the 4E10 epitope and corresponds to the W670-W678 consensus group M sequence of gp160. The structure of this complex elucidates the epitope conformation recognized by 4E10, as well as its interaction with this neutralizing antibody.

Peptides also appear to be good candidates in the development of a vaccine against HIV. Carrier-conjugated synthetic peptides have advantages over protein-based systems because peptides can be modified and synthesized more easily than proteins, therefore they can be used more readily in a drug design process. Moreover, synthetic peptides, conjugated to the appropriate carrier elicit antibodies that often cross react with the native protein antigen.

The success of immunoprophylaxis in animal models using HIV-1 neutralizing monoclonal antibodies suggests that, if neutralizing antibodies could be generated by an appropriate vaccine, they could provide substantial benefits (Gauduin et al., 1997; Parren et al., 2001; Ferrantelli et al., 2002; Ferrantelli et al., 2003; Mascola, 2003). However, the goal of designing immunogens which elicit antibodies that can neutralize multiple isolates of HIV-1 has been extraordinarily difficult to achieve. The vast majority of anti-HIV-1 antibodies elicited either by immunization or during natural infection have poor or no cross-neutralizing activity to other HIV-1 isolates and typically bind to epitopes that either vary from virus to virus or are poorly, or not, exposed on infectious virions.

The present invention identifies, designs and synthesizes peptides and peptidomimetics that would target more than one epitope present on gp41 using information on the structure of 4E10 and 2F5/peptide complexes that can ultimately be used in therapeutics or vaccines.

### SUMMARY OF THE INVENTION

The structural features of antibody (Fab) 4E10, the broadest HIV nAB (neutralizing antibody), complexed with KGND, have been discovered from analysis of its crystal structure. It has also been discovered that the structure of KGND, 4E10 mimetope on gp41, has a functionally relevant conformation; that is, the structure of KGND—a helical structure—has been elucidated. This structure provides information on how compounds can bind to 4E10, as well as on how compounds may bind to KGND. Also, the interaction of key residues (e.g., Trp5, Phe6, Ile8, and Thr9) on 4E10/4E10 epitope have been determined. The atomic coordinates of the crystal structure are set forth in Table 1. The crystal features are: a C2 space group, cell parameters (in angstroms for a, b, c and degrees for Beta, rms deviations 0.5 angstroms, 1.0 degrees) of a: 157.3 angstroms, b: 45.1 angstroms, c: 198.6 angstroms, and Beta: 113.8 degrees. There are two dimers (i.e. Fab-peptide) per asymmetric unit. Other aspects of the crystal structure are provided in the Figures and Table 1.

The invention thus provides a Fab 4E10:KGND complex having the crystal structure herein described, e.g., a C2 space group, cell parameters (in angstroms for a, b, c and degrees for Beta, rms deviations 0.5 angstroms, 1.0 degrees) of a: 157.3 angstroms, b: 45.1 angstroms, c: 198.6 angstroms, and Beta: 113.8 degrees and/or having an X-ray diffraction pattern corresponding to or resulting from any or all of the foregoing and/or having an X-ray diffraction pattern corresponding to or resulting from any or all of the foregoing and/or a crystal having the structure defined by the coordinates of Table 1. Furthermore, one of skill in the art will recognize that using the coordinates of Table 1, it is possible to obtain multiple crystal structures which may crystallize in another space group with differing cell dimensions. The invention encompasses such other structures and uses thereof as herein discussed.

The invention further provides a peptide which consists essentially of WFXIT (SEQ ID NO: 78), wherein X may be N, D, S, G or other amino acids, e.g., conservative substitutions thereof. WFXIT (SEQ ID NO: 78) has been identified as the key residues of 4E10. These residues may be flanked on either side, however the present invention does not encompass such sequences as known in the art, or which would alter the structure (from the helical structure elucidated as part of this invention). Furthermore, the invention encompasses a polypeptide having a sequence consisting essentially of DKWX¹X²X³X⁴X⁵WFXIT (SEQ ID NO: 3), wherein X is as defined above, X¹=A or a conservative substitution thereof, X²=N or a conservative substitution thereof, X³=L or a conservative substitution thereof, X⁴=W or a conservative substitution thereof, X⁵=N or a conservative substitution thereof, wherein the polypeptide has a helical structure, and it is not otherwise disclosed in the art. X⁵ can also be S or T or conservative substitutions thereof. In one embodiment, the peptide binds to Fab 4E10.

Yet further still, the invention also encompasses a polypeptide having a sequence consisting essentially of DKWX¹X²X³X⁴X⁵WFXIT (SEQ ID NO: 3), wherein X=N, D, S, G, Q, C, T, M, E, K, R, A, P, I, L, V, Ornithine (hereinafter “O”), Aib, or other natural or synthetic amino acids, including conservative substitutions thereof, X¹=A, G, P, I, L, V, Aib, or other natural or synthetic amino acids, or a conservative substitution thereof; X²=N, Q, C, S, T, M, or other natural or synthetic amino acids, or a conservative substitution thereof; X³=L, I, V, G, A, P, or other natural or synthetic amino acids, or a conservative substitution thereof, X⁴=W, H, F, Y, K, C, Aib, or other natural or synthetic amino acids, or a conservative substitution thereof, X⁵=N, S, T, Q, C, M, E, A, or other natural or synthetic amino acids, or a conservative substitution thereof; and wherein the polypeptide has a helical structure. In one embodiment, the peptide binds to Fab 4E10.

Yet even further still, the invention also encompasses a polypeptide having a sequence consisting essentially of DKWX¹X²X³X⁴X⁵WFXITXX⁶XW (SEQ ID NO: 4), wherein X=N, D, S, G, Q, C, T, M, E, K, R, A, P, I, L, V, O, Aib, or other natural or synthetic amino acids, including conservative substitutions thereof, X¹=A, G, P, I, L, V, Aib, or other natural or synthetic amino acids, or a conservative substitution thereof; X²=N, Q, C, S, T, M, or other natural or synthetic amino acids, or a conservative substitution thereof; X³=L, I, V, G, A, P, or other natural or synthetic amino acids, or a conservative substitution thereof, X⁴=W, H, F, Y, K, C, Aib, or other natural or synthetic amino acids, or a conservative substitution thereof, X⁵=N, S, T, Q, C, M, E, A, or other natural or synthetic amino acids, or a conservative substitution thereof, X⁶=any natural or synthetic amino acids; and wherein the polypeptide has a helical structure. In one embodiment, the peptide binds to Fab 4E10. In one embodiment, X⁶ is W, such that the polypeptide has the sequence consisting essentially of DKWX¹X²X³X⁴X⁵WFXITXWXW (SEQ ID NO: 5). For example, a peptide with this sequence is shown in FIG. 40C.

The invention further encompasses a polypeptide having a sequence consisting essentially of XNWFX¹ITX²WLWX (SEQ ID NO: 6), wherein X comprises 0-8 amino acids consisting essentially of K, Aib, Y, I, or other natural or synthetic amino acids, including conservative substitutions thereof; wherein X¹=D, C, or other natural or synthetic amino acids, including conservative substitutions thereof; wherein X²=0, N, or other natural or synthetic amino acids or a conservative substitution thereof, wherein the polypeptide has a helical structure, and is not otherwise disclosed in the art. In some embodiments, Aib may be inserted between any two amino acids of WFX¹IT (SEQ ID NO: 79). Alternatively or additionally, WFX¹IT (SEQ ID NO: 79) can be branched. The branched chain can be of sufficient length and/or configuration that the polypeptide binds to Fab 4E10. In another embodiment, the polypeptide comprises or consists essentially of: NWFCITOWLWKKKK-NH₂ (SEQ ID NO: 7); NWFDITNWLWYIKKKK-NH₂ (SEQ ID NO: 8); NWFDITNWLWK-Aib-K-Aib-K-NH₂ (SEQ ID NO: 9); KK-Aib-NWFDITNWLWK-Aib-K-Aib-K-NH₂ (SEQ ID NO: 10); NWFDITNWLWYIK-Aib-K-Aib-KK-NH₂ (SEQ ID NO: 11); or NWFCITOWLWKKKK-NH₂ (SEQ ID NO: 12).

The invention additionally encompasses a polypeptide having a sequence consisting essentially of: NWFX¹ITX²WLWX (SEQ ID NO: 13), wherein X comprises 0-8 amino acids consisting essentially of K, Aib, Y, I, or other natural or synthetic amino acids, including conservative substitutions thereof; wherein X¹=D, C, or other natural or synthetic amino acids or a conservative substitution thereof; wherein X²=0, N, or other natural or synthetic amino acids or a conservative substitution thereof; wherein the polypeptide has a helical structure, and is not otherwise disclosed in the art.

The invention further encompasses a polypeptide having a sequence consisting essentially of WFX(I/L)(T/S)XX(L/I)W wherein X does not play a major role in Fab 4E10 binding. The polypeptide may have a helical structure and X may further introduces constraints (e.g., Aib). Advantageously, the polypeptide binds to Fab 4E10.

The invention also provides a method for screening or identification comprising exposing the Fab 4E10 of the foregoing crystal structure to one or more test samples, and determining whether a Fab 4E10 complex is formed. The method can be performed wherein the Fab 4E10 or functional portion thereof is exposed to the test samples by co-crystallizing the Fab 4E10 protein or functional portion thereof in the presence of the one or more test samples. The resulting crystals can be analyzed by X-ray diffraction or crystallographic techniques and compared with the herein data. If similar in crystal structure, the test sample thus binds to Fab 4E10 in a manner analogous to KGND, and is thus useful for eliciting antibodies or in a diagnostic, pharmaceutical immunogenic, immunological or vaccine composition. The Fab 4E10 can be soaked in a solution of one or more test samples. These methods may also be used with other, similiarly binding Mabs, including, but not limited to, Z13, in order to determine whether a test sample will crystallize with the Z13 or other Mab.

The invention also provides a computer-assisted method for identifying or designing potential compounds to fit within or bind to Fab 4E10 or a functional portion thereof: comprising using a computer system, e.g., a programmed computer comprising a processor, a data storage system, an input device, and an output device, the steps of: (a) inputting into the programmed computer through said input device data comprising the three-dimensional coordinates of a subset of the atoms in the Fab 4E10 binding domain (containing or binding to key residues identified herein), optionally with structural information from Fab 4E10 complex(es), such as the Fab 4E10:KGND complex, thereby generating a data set; (b) comparing, using said processor, said data set to a computer database of chemical structures stored in said computer data storage system; (c) selecting from said database, using computer methods, chemical structures having a portion that is structurally similar to said data set; (d) constructing, using computer methods, a model of a chemical structure having a portion that is structurally similar to said data set and (e) outputting to said output device the selected chemical structures having a portion similar to said data set; and optionally synthesizing one or more of the selected chemical structures; and further optionally contacting said synthesized selected chemical structure with Fab 4E10 to ascertain whether said synthesized chemical structure binds to or fits within the domain of Fab 4E10 and/or administering said chemical structure to an animal capable of having an antibody response to ascertain whether the chemical structure elicits anti-HIV antibodies (e.g., by testing said resultant antibodies for binding to HIV or HIV glycoproteins or portions thereof); or, comprising: providing the structure of Fab 4E10 as defined by the co-ordinates of Table 1, providing the structure of a candidate binding molecule, and fitting the structure of the candidate to the structure of the Fab 4E10 of Table 1; or, comprising: providing the co-ordinates of at least two atoms of Table 1 of Fab 4E10 (“selected co-ordinates”), providing the structure of a candidate binding molecule, and fitting the structure of the candidate to the selected coordinates; or, comprising: providing the co-ordinates of at least a sub-domain of Fab 4E10, providing the structure of a candidate binding molecule, and fitting the structure of the candidate to the sub-domain of Fab 4E10; said method optionally further comprising: obtaining or synthesizing the chemical structure or candidate and contacting the chemical structure or candidate with Fab 4E10 to determine the ability of the chemical structure or candidate to interact with Fab 4E10; or obtaining or synthesizing the chemical structure or candidate and forming a complex of Fab 4E10 and said chemical structure or candidate, and analyzing the complex to determine the ability of said chemical structure or candidate to interact with Fab 4E10 and/or administering said chemical structure or candidate to an animal capable of raising antibodies against the chemical structure to ascertain whether said chemical structure or candidate elicits anti-HIV antibodies (e.g., by testing said resultant antibodies for binding to HIV or HIV glycoproteins or portions thereof).

And these methods or steps thereof optionally include transmission of information from such methods or steps, e.g., via telecommunication, telephone, video conference, mass communication, e.g., presentation such as a computer presentation (e.g. POWERPOINT), interne, email, documentary communication such as a computer program (e.g. WORD) document and the like.

The invention further comprehends a compound having a chemical structure selected using the herein methods, said compound binding to Fab 4E10 and eliciting an anti-HIV antibody. The invention further still comprehends compositions containing such a compound, e.g., a diagnostic, pharmaceutical, immunogenic, immunological, or vaccine composition, as well as methods for making and using such compositions, e.g., admixing such compound with a pharmaceutically suitable or acceptable vehicle or carrier or diluent, including and/or adjuvant when desired; administering to an animal that generates antibodies the compound or composition, for instance, to generate anti-HIV antibodies that may be diagnostically useful or an immunogenic or immunological or vaccine response (for instance, if the animal is susceptible to HIV, such as a human, so as to provide a prophylactic or treatment); or, using the compound to detect the presence of anti-HIV antibodies in a sample (for instance, by labeling the compound and detecting binding of the compound and hence anti-HIV antibodies).

The invention further relates to identification, design, synthesis and isolation of the polypeptide herein referred to as KGND, which has the sequence set forth in FIG. 9. The present invention also relates to homologues, derivatives and variants of KGND. Yet further still, the invention relates to the conformational structure of KGND, as described herein. Furthermore, it is assumed that any homologues, derivatives and variants of KGND would encompass the conformational structure of KGND as described herein. Additionally, the invention relates to nucleic acids encoding KGND or homologues, derivative or variants of KGND, as wells as to vectors comprising and expressing such nucleic acids.

The invention also provides a method for screening or identification comprising exposing the KGND binding domain of the antibody of the foregoing crystal structure to one or more test samples, and determining whether a KGND antibody complex is formed. The method can be performed wherein the KGND binding domain of the antibody or functional portion thereof is exposed to the test samples by co-crystallizing the antibodies or functional portions thereof in the presence of the one or more test samples (KGND analogs). The resulting crystals can be analyzed by X-ray diffraction or crystallographic techniques and compared with the herein data.

If similar in crystal structure, the test sample thus binds to Fab 4E10 in a manner analogous to KGND, and is thus useful for eliciting antibodies or in a diagnostic, pharmaceutical immunogenic, immunological or vaccine composition. The antibodies or functional portions can be soaked in a solution of one or more test samples. These methods may also be used with other, similiarly binding Mabs, including, but not limited to, Z13, in order to determine whether a test sample will crystallize with the Z13 or other Mab.

The invention also provides a computer-assisted method for identifying or designing potential compounds to fit within or bind to the KGND binding domain of the antibody or a functional portion thereof: comprising using a computer system, e.g., a programmed computer comprising a processor, a data storage system, an input device, and an output device, the steps of: (a) inputting into the programmed computer through said input device data comprising the three-dimensional co-ordinates of a subset of the atoms in the KGND antibody binding domain (containing or binding to key residues identified herein), optionally with structural information from KGND antibody complex(es), such as the Fab 4E10:KGND complex, thereby generating a data set; (b) comparing, using said processor, said data set to a computer database of chemical structures stored in said computer data storage system; (c) selecting from said database, using computer methods, chemical structures having a portion that is structurally similar to said data set; (d) constructing, using computer methods, a model of a chemical structure having a portion that is structurally similar to said data set and (e) outputting to said output device the selected chemical structures having a portion similar to said data set; and optionally synthesizing one or more of the selected chemical structures; and further optionally contacting said synthesized selected chemical structure with the KGND domain of the antibody or a functional portion to ascertain whether said synthesized chemical structure binds to or fits within the domain of KGND and/or administering said chemical structure to an animal capable of having an antibody response to ascertain whether the chemical structure elicits anti-HIV antibodies (e.g., by testing said resultant antibodies for binding to HIV or HIV glycoproteins or portions thereof); or, comprising: providing the structure of KGND as defined by the co-ordinates of Table 1, providing the structure of a candidate binding molecule, and fitting the structure of the candidate to the structure of the KGND of Table 1; or, comprising: providing the co-ordinates of at least two atoms of Table 1 of KGND (“selected co-ordinates”), providing the structure of a candidate binding molecule, and fitting the structure of the candidate to the selected co-ordinates; or, comprising: providing the co-ordinates of at least a sub-domain of KGND, providing the structure of a candidate binding molecule, and fitting the structure of the candidate to the sub-domain of KGND; said method optionally further comprising: obtaining or synthesizing the chemical structure or candidate and contacting the chemical structure or candidate with KGND antibody binding domain to determine the ability of the chemical structure or candidate to interact with the KGND antibody binding domain; or obtaining or synthesizing the chemical structure or candidate and forming a complex of the KGND antibody binding domain and said chemical structure or candidate, and analyzing the complex to determine the ability of said chemical structure or candidate to interact with the KGND antibody binding domain and/or administering said chemical structure or candidate to an animal capable of raising antibodies against the chemical structure to ascertain whether said chemical structure or candidate elicits anti-HIV antibodies (e.g., by testing said resultant antibodies for binding to HIV or HIV glycoproteins or portions thereof).

And these methods or steps thereof optionally include transmission of information from such methods or steps, e.g., via telecommunication, telephone, video conference, mass communication, e.g., presentation such as a computer presentation (e.g., POWERPOINT), internet, email, documentary communication such as a computer program (e.g., WORD) document and the like.

The invention further comprehends a compound having a chemical structure selected using the herein methods, said compound binding to the KGND antibody binding domain and eliciting an anti-HIV antibody. The invention further still comprehends compositions containing such a compound, e.g., a diagnostic, pharmaceutical, immunogenic, immunological, or vaccine composition, as well as methods for making and using such compositions, e.g., admixing such compound with a pharmaceutically suitable or acceptable vehicle or carrier or diluent, including and/or adjuvant when desired; administering to an animal that generates antibodies the compound or composition, for instance, to generate anti-HIV antibodies that may be diagnostically useful or an immunogenic or immunological or vaccine response (for instance, if the animal is susceptible to HIV, such as a human, so as to provide a prophylactic or treatment); or, using the compound to detect the presence of anti-HIV antibodies in a sample (for instance, by labeling the compound and detecting binding of the compound and hence anti-HIV antibodies).

The invention also comprises a diagnostic, pharmaceutical, immunogenic, immunological, or vaccine composition containing a polypeptide of the present invention.

The invention also describes a method for making a composition comprising a polypeptide of the present invention, wherein the method comprises admixing such polypeptide with a pharmaceutically suitable or acceptable vehicle or carrier or diluent, optionally including or being an adjuvant.

The invention further encompasses a method for using a composition according to the invention, wherein the composition is administered to an animal that generates antibodies to the composition, wherein the antibodies generated are anti-HIV antibodies that may be diagnostically useful or wherein administration of the composition elicits an immunogenic or immunological or vaccine response; or, where the composition is used to detect the presence of anti-HIV antibodies in a sample.

Also provided by the present invention is a method for eliciting anti-HIV antibodies comprising administering to an animal capable of eliciting antibodies a composition of the present invention.

A method for detecting anti-HIV antibodies is provided, comprising contacting a sample suspected of having such antibodies with a composition of the invention and detecting binding of the antibody to the composition. In one embodiment, the animal is a human and the method is for treatment or prevention of HIV. In another embodiment, the method is for generating antibodies for diagnostic purposes.

Further provided herein is a diagnostic composition containing a polypeptide of the invention or an antibody elicited by administration of the polypeptide. The invention also encompasses a composition for prevention or treatment of HIV, comprising a polypeptide of the invention, or an antibody elicited by administration of the polypeptide. In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

These and other embodiments are disclosed or are obvious from and encompassed by, the following Detailed Description.

### BRIEF DESCRIPTION OF THE DRAWINGS

The following Detailed Description, given to describe the invention by way of example, but not intended to limit the invention to specific embodiments described, as well as the foregoing text, may be understood in conjunction with the accompanying Figures, incorporated herein by reference, in which:

FIG. 1 shows the HIV-1 envelope glycoproteins gp120 and gp41;

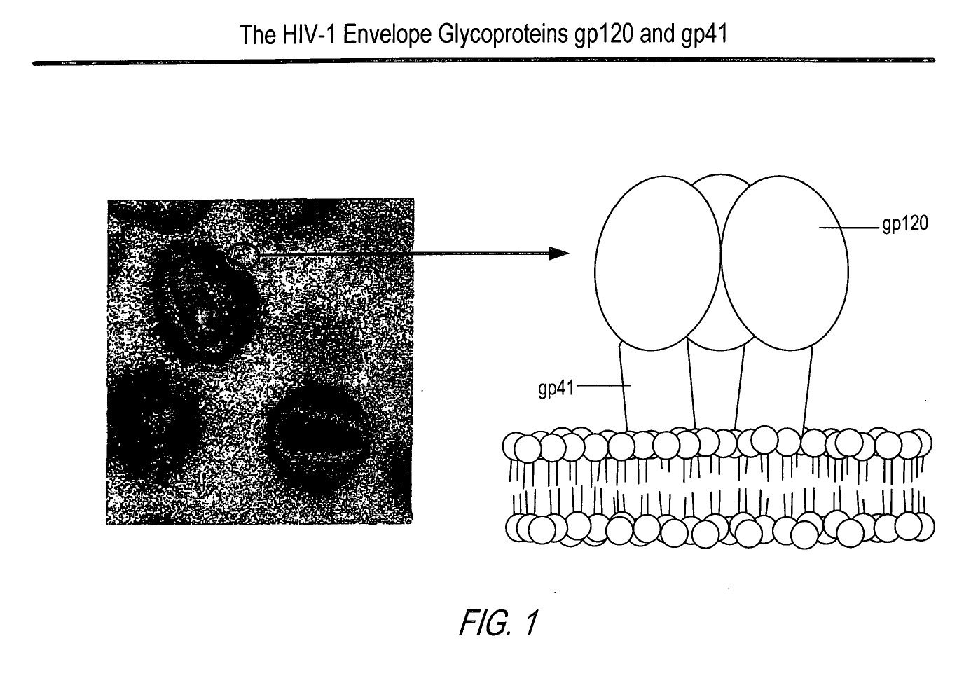

FIG. 2 shows the structure of gp120 core as a complex;

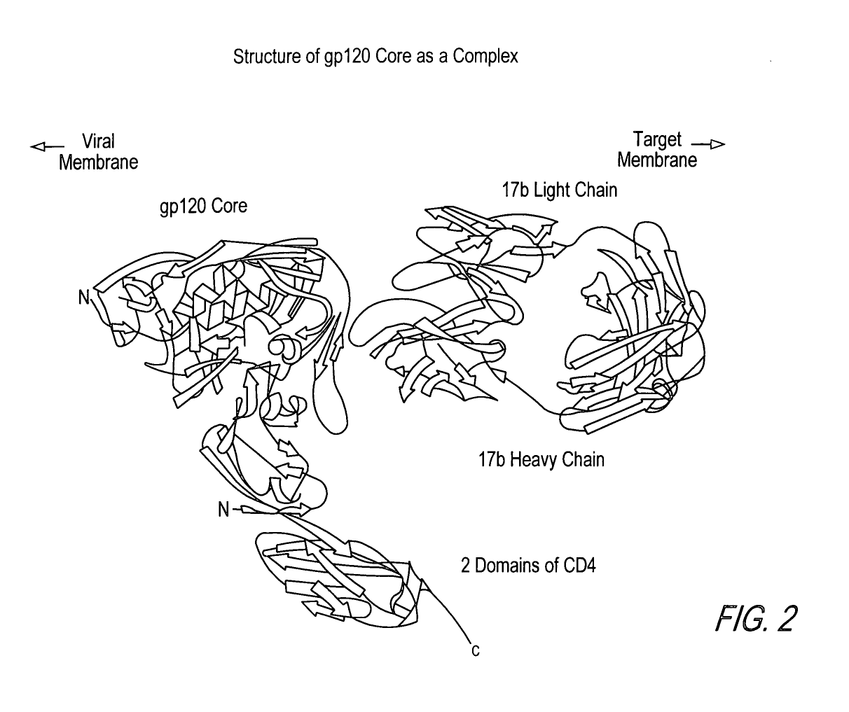

FIG. 3 shows the structure of gp120 core;

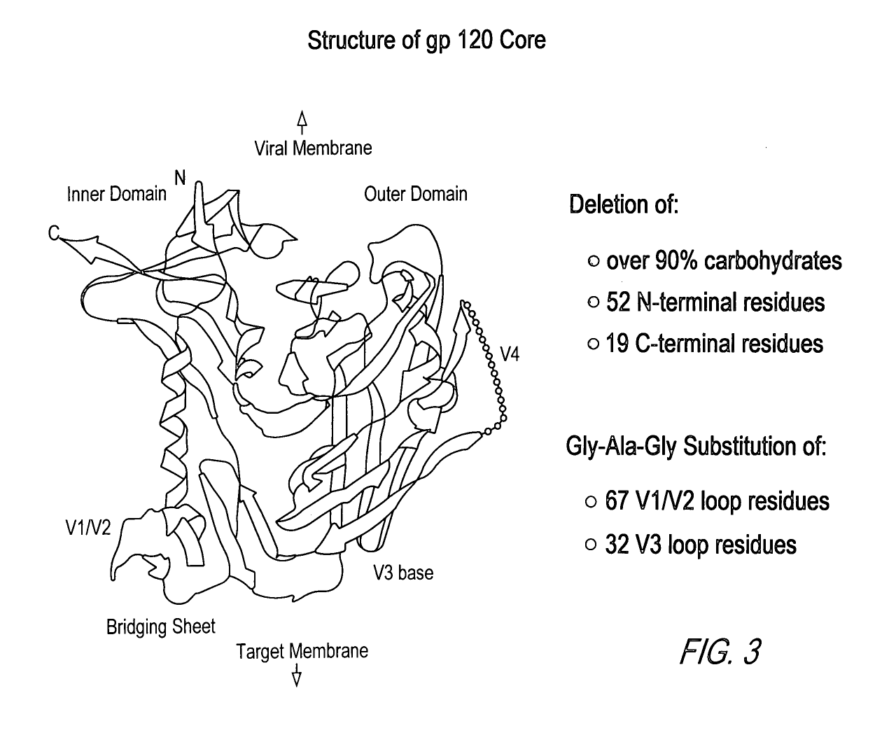

FIG. 4 shows the structure of gp41 core in the fusogenic state;

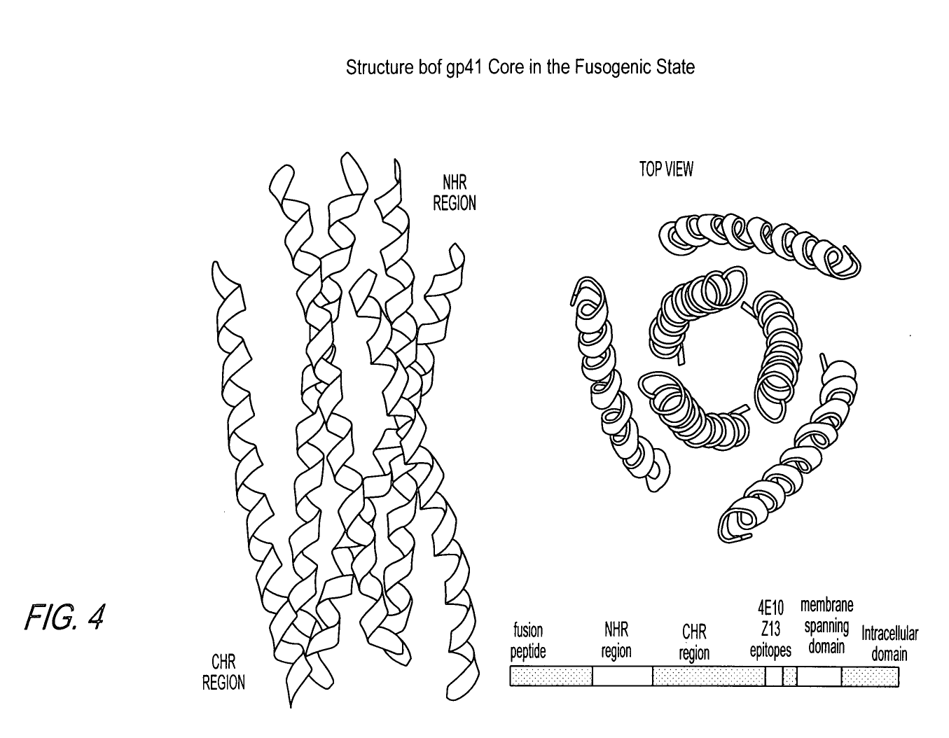

FIG. 5 shows epitopes of HIV-1 neutralizing antibodies (nabs) on gp120 and gp41;

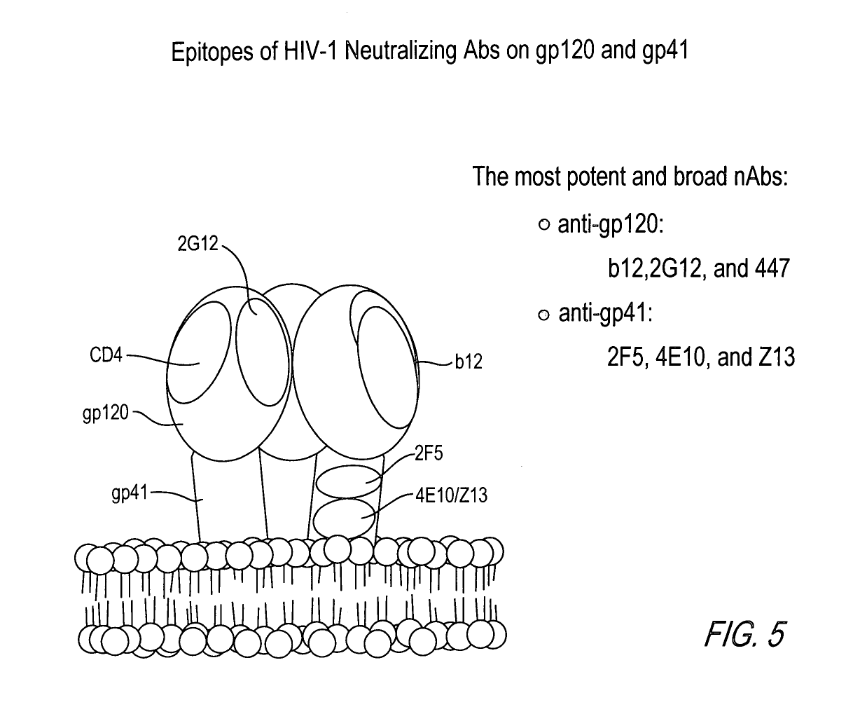

FIG. 6 shows binding of anti-gp41 Fabs to immobilized gp41 by ELISA;

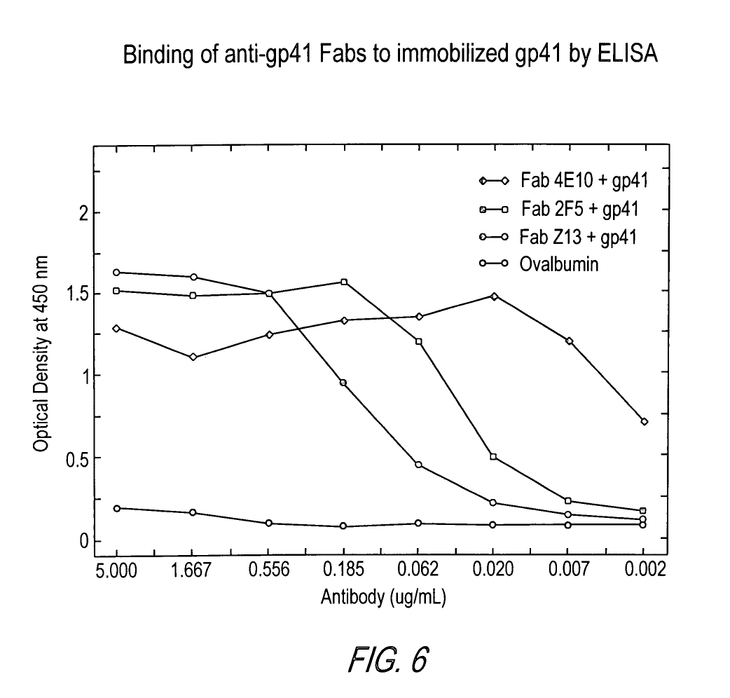

FIG. 7 shows the production of Fab 4E10;

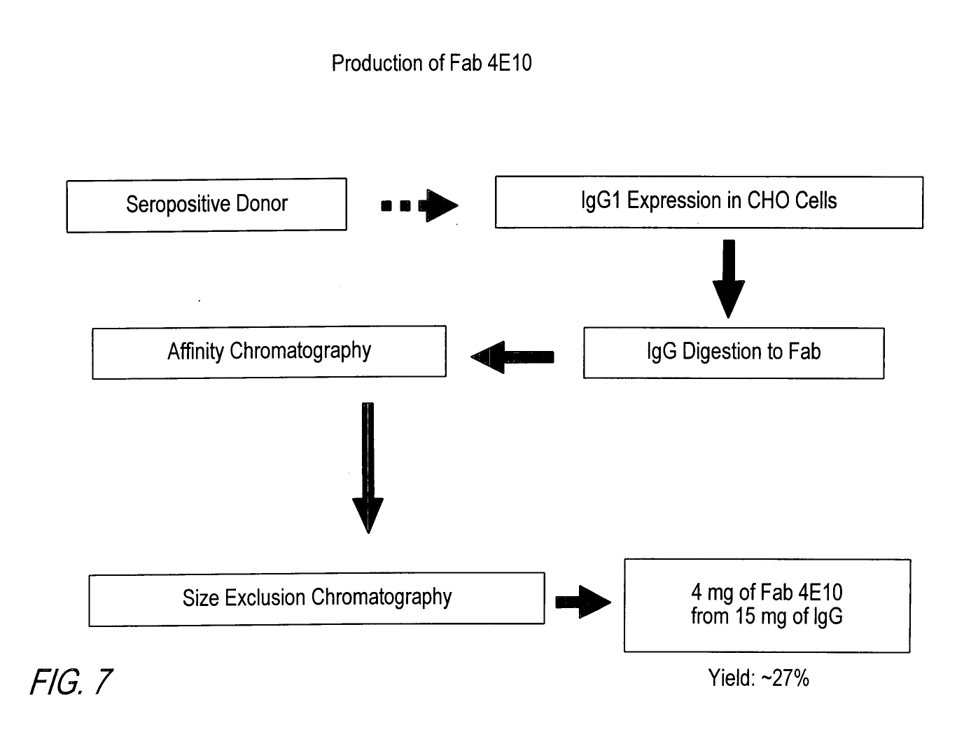

FIG. 8 shows the purification of Fab 4E10—size exclusion chromatography, superdex 75 16/60 chromatograph, NR 4-20% SDS-PAGE;

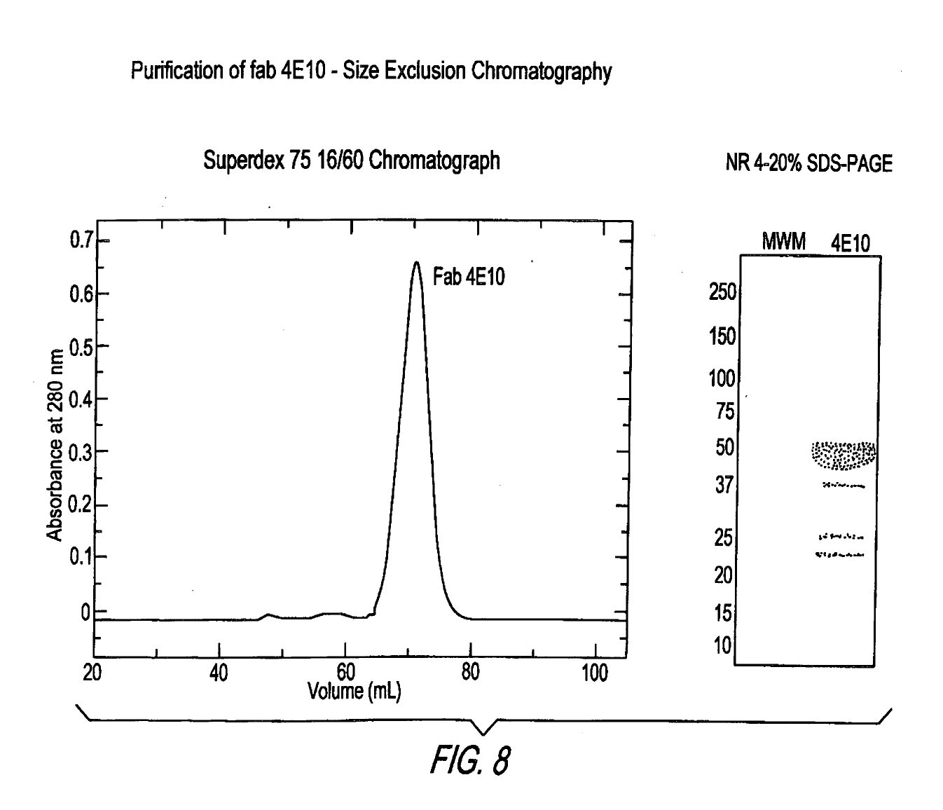

FIG. 9 shows peptide KGND (disclosed as SEQ NO: 2), a 4E10 mimetope on gp41 (in the 4E10 epitope, the gp41 sequence can be prefaced by LLELDKWA (SEQ ID NO: 81), and the K in the sequence depicted may be an N, i.e., SLWNWFDITNWLW (SEQ ID NO: 14));

FIG. 10 shows Fab 4E10 binding to immobilized peptide KGND by ELISA;

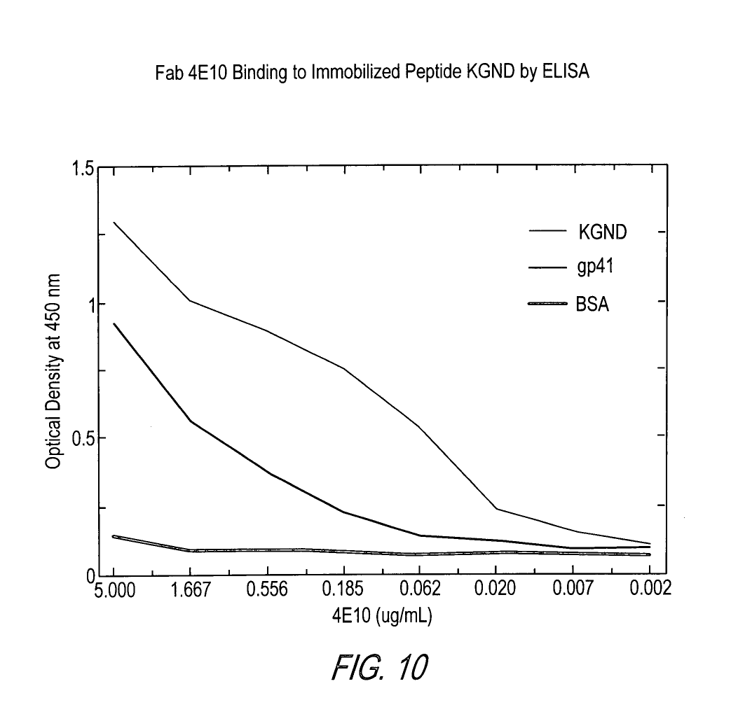

FIG. 11 shows peptide KGND complex crystallographic results, quadrant of the X-ray diffraction pattern;

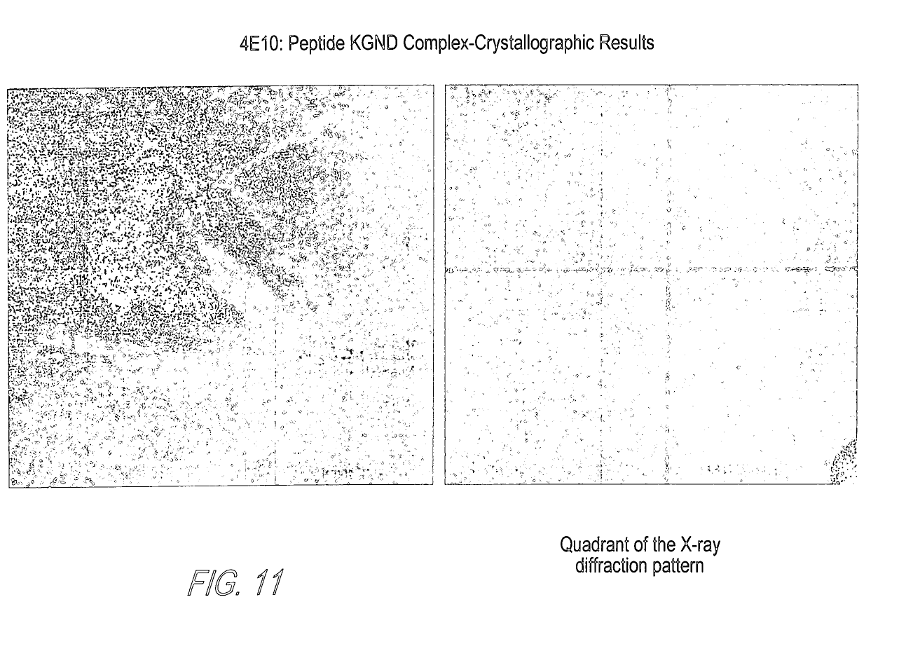

FIG. 12 shows Fab4E10:KGND complex data processing statistics;

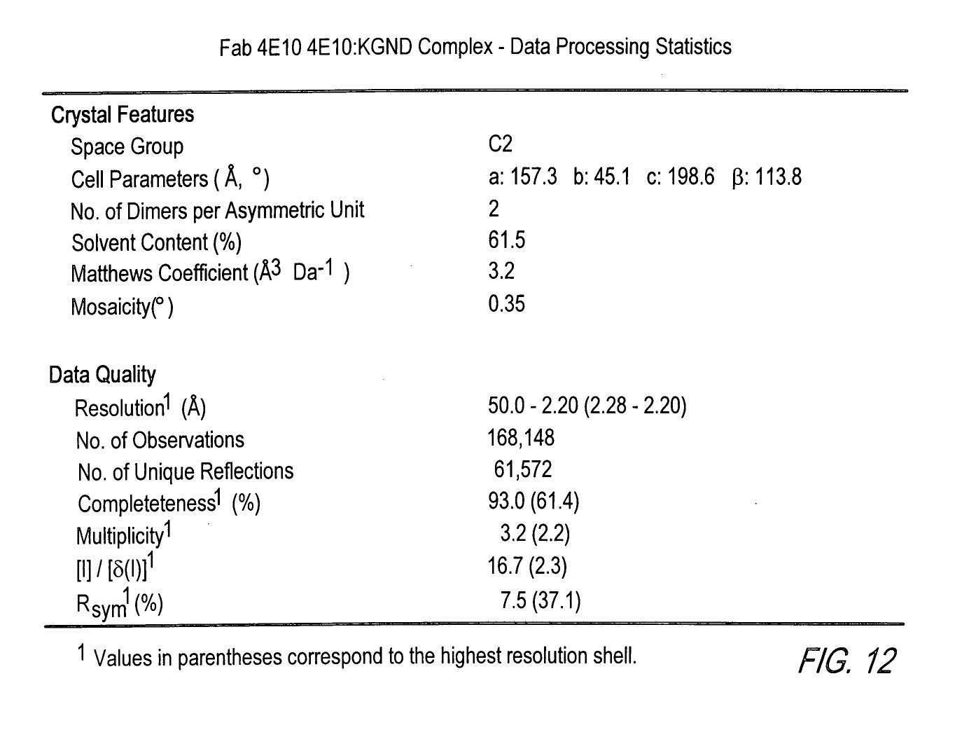

FIG. 13 shows Fab4E10:KGND complex refinement statistics;

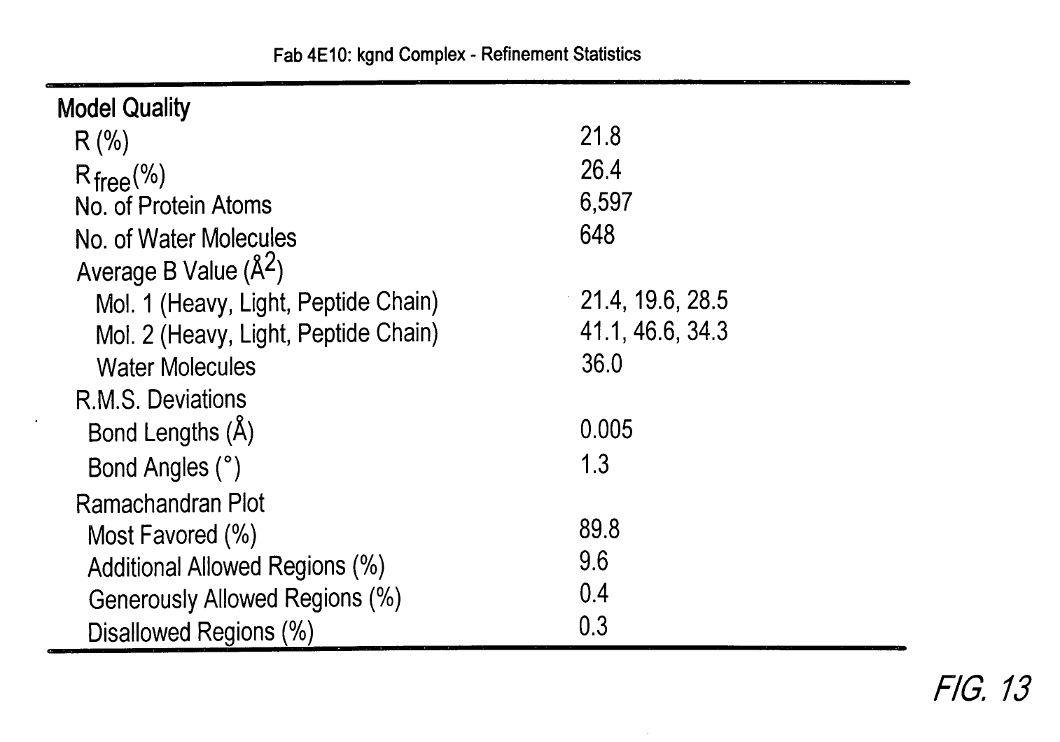

FIG. 14 shows the electron density of KGND peptide with Fab 4E10 at 2.2 angstroms;

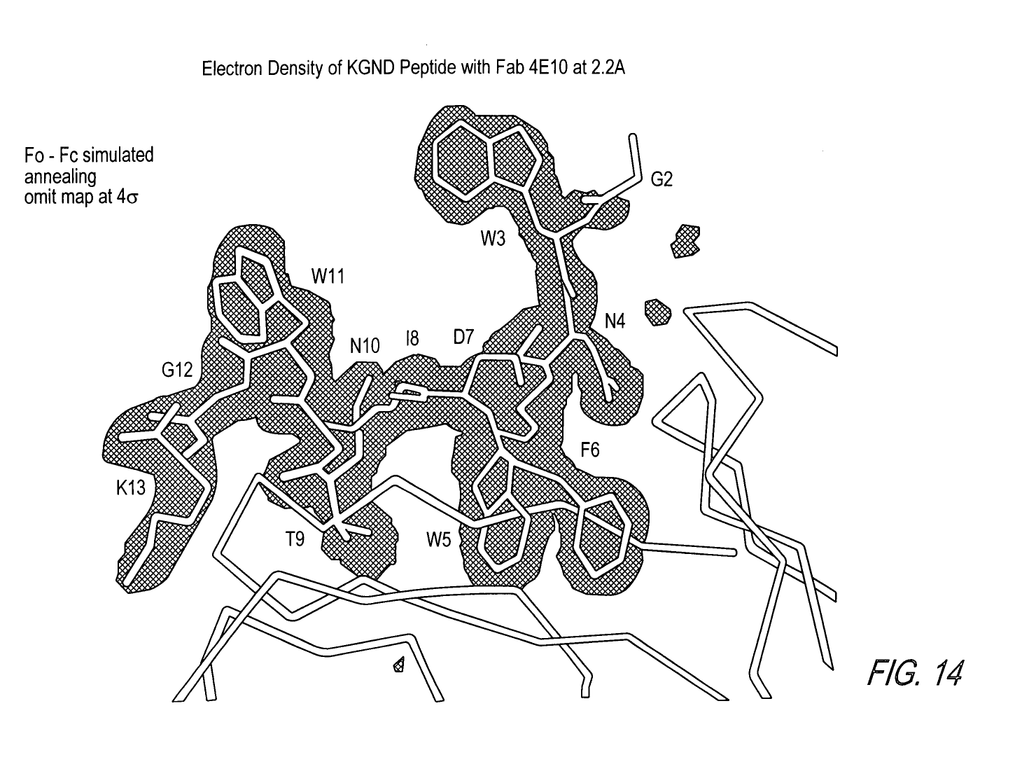

FIG. 15 shows a global view of Fab 4E10 in complex with peptide KGND;

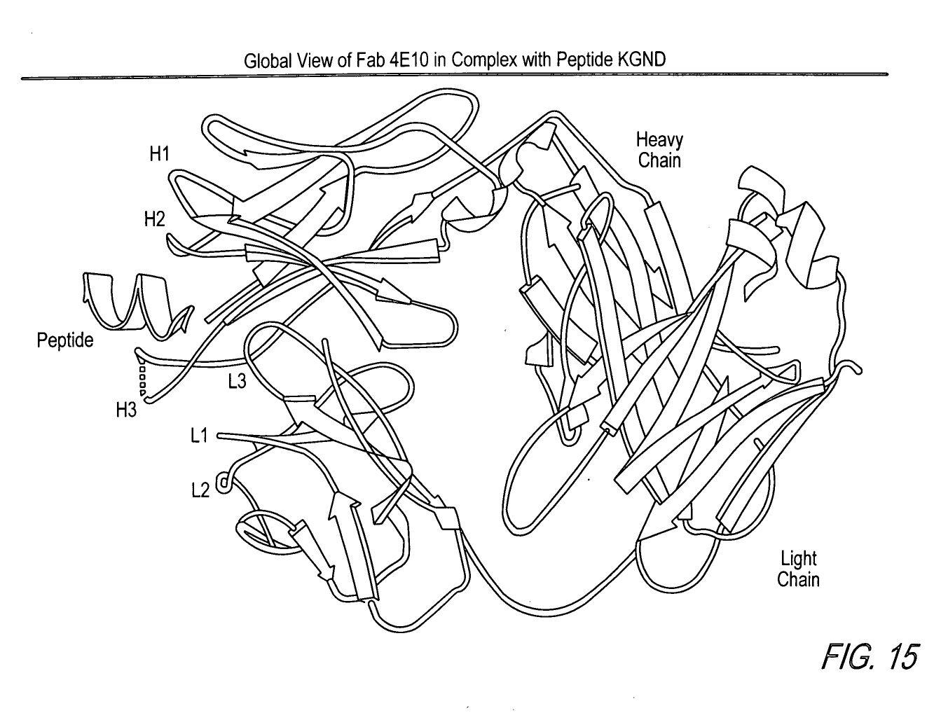

FIG. 16 shows peptide KGND (disclosed as SEQ ID NO: 2);

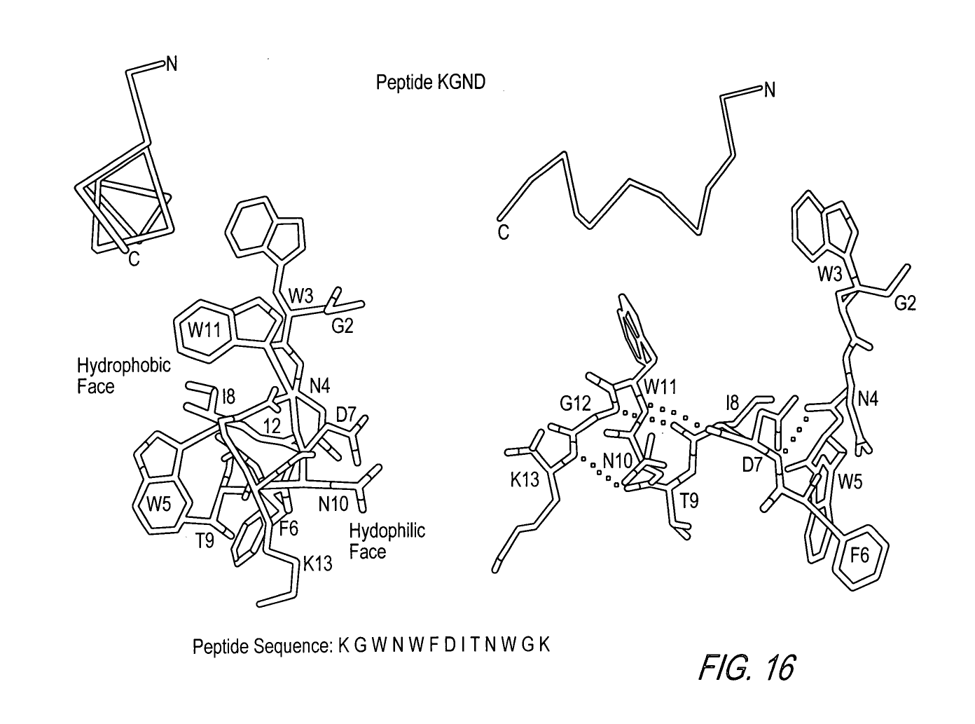

FIG. 17 shows a top view of peptide KGND (disclosed as SEQ ID NO: 2);

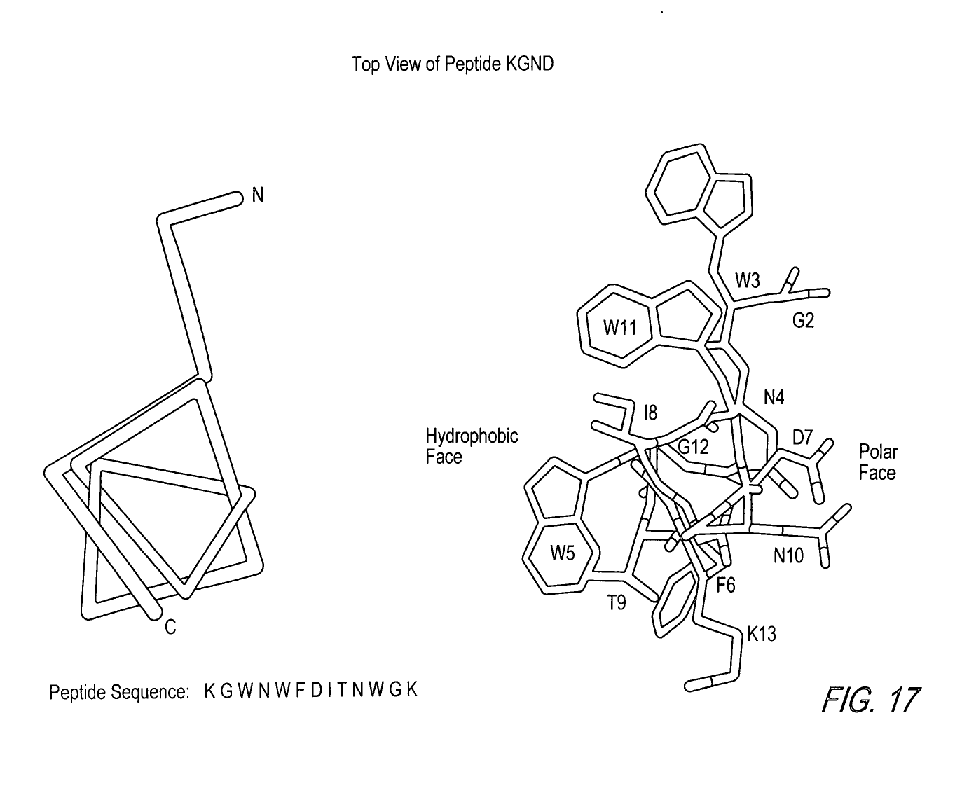

FIG. 18 shows a side view of peptide KGND (disclosed as SEQ ID NO: 2);

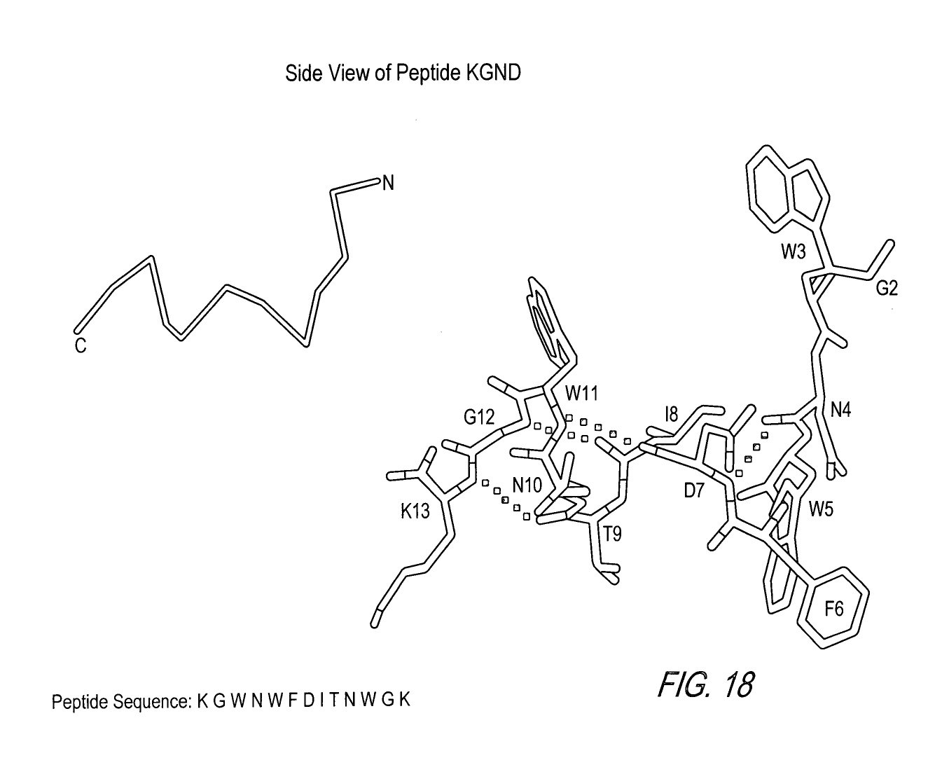

FIG. 19 shows Fab 4E10 in complex with peptide KGND;

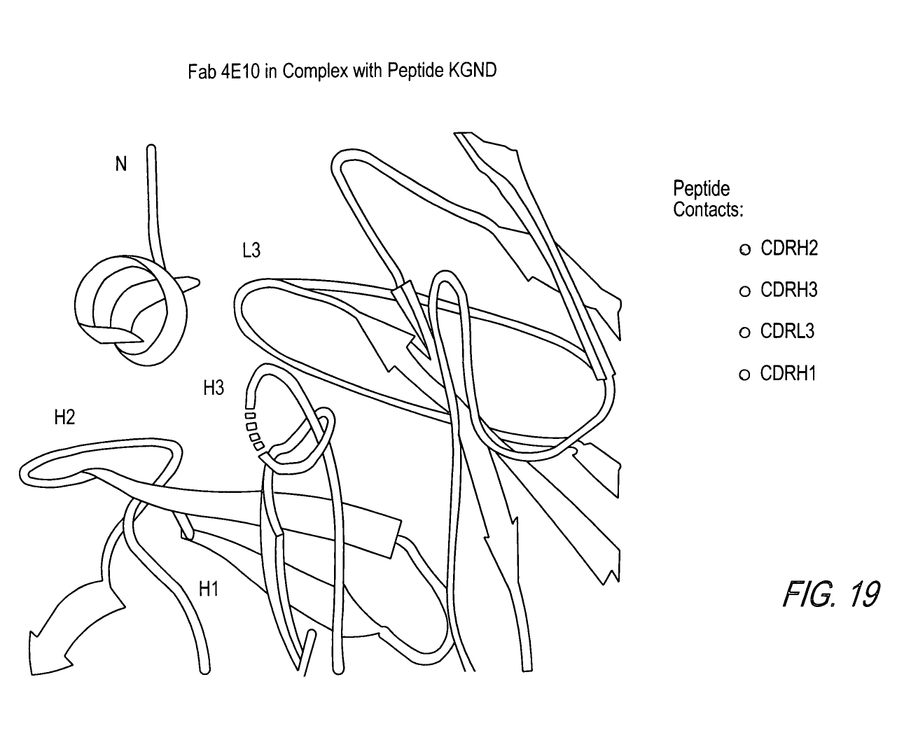

FIG. 20 shows Fab 4E10 in complex with peptide KGND, induced fit;

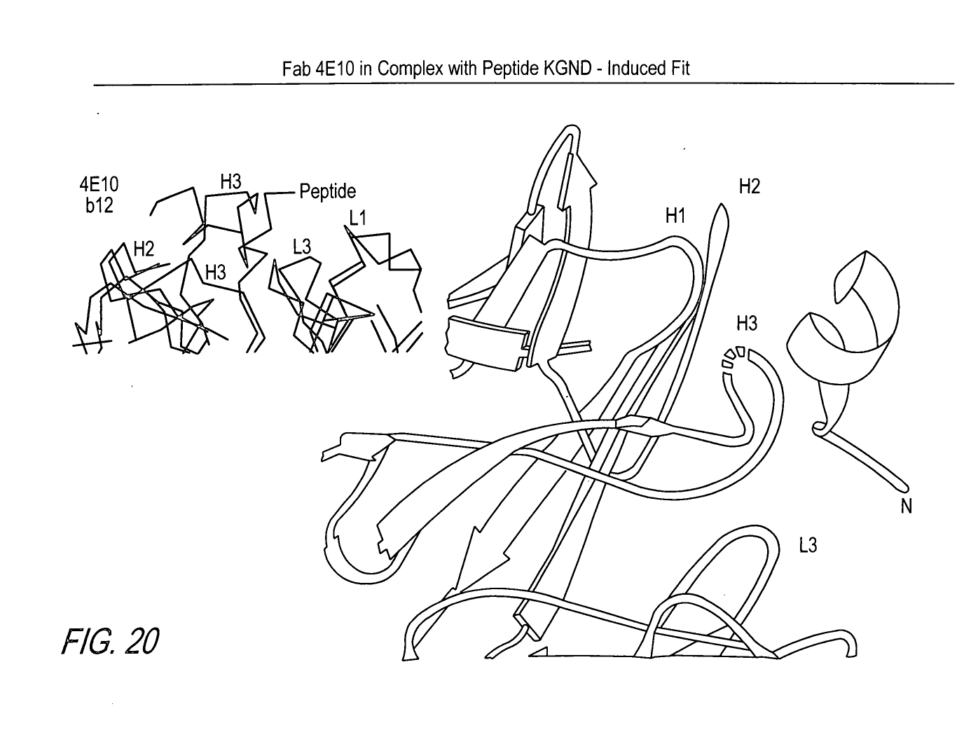

FIG. 21 shows 4E10:KGND complex, electrostatic potential surface;

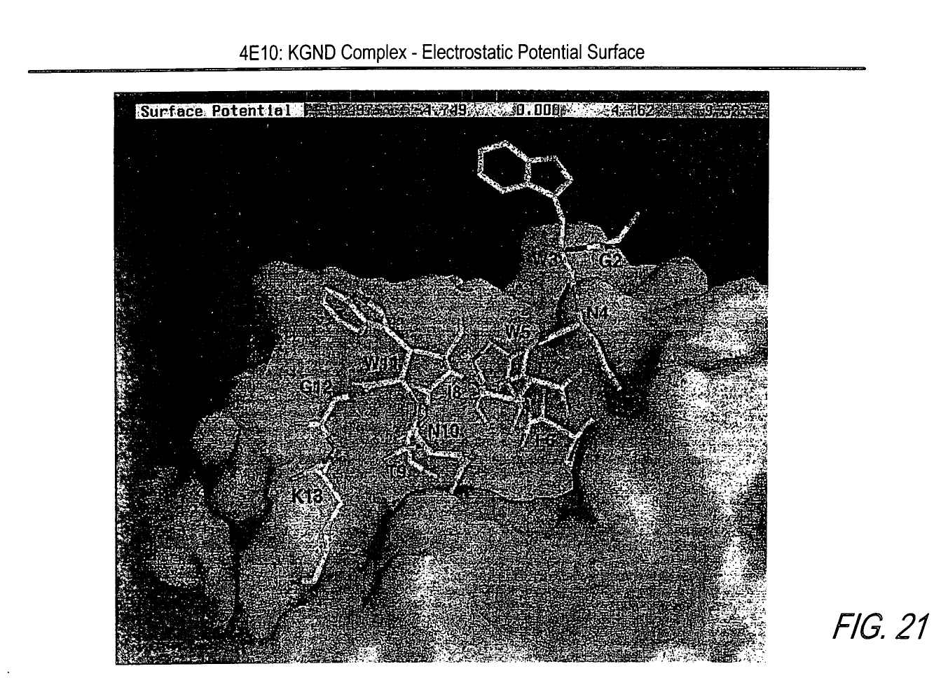

FIG. 22 shows 4E10:KGND complex, Trp3 and Trp11 crystal contacts;

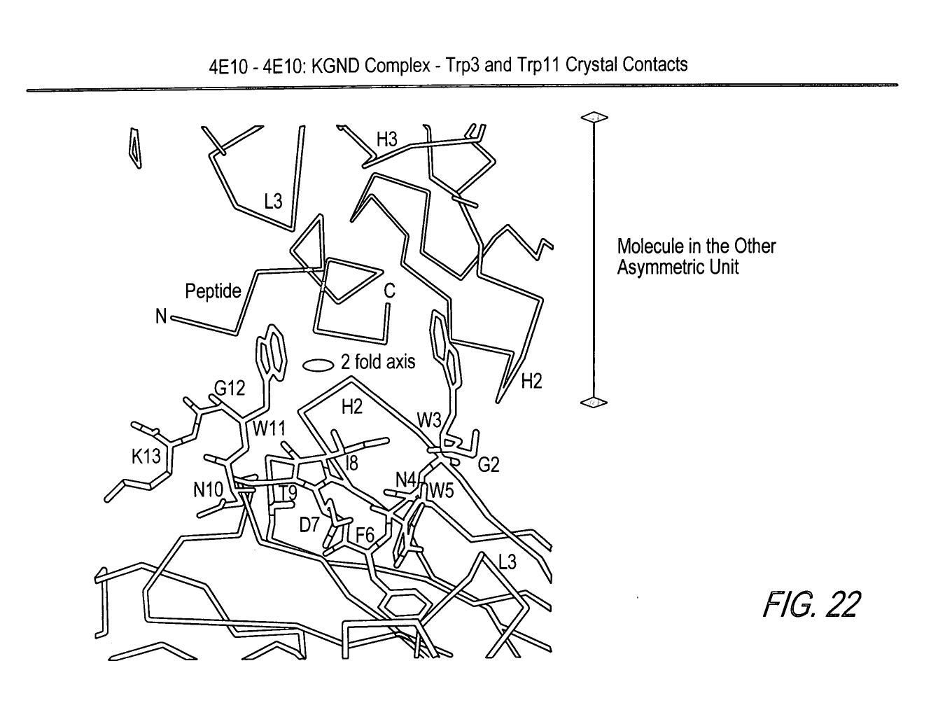

FIG. 23 shows 4 E10:KGND complex, Trp3 and Trp11 crystal contacts;

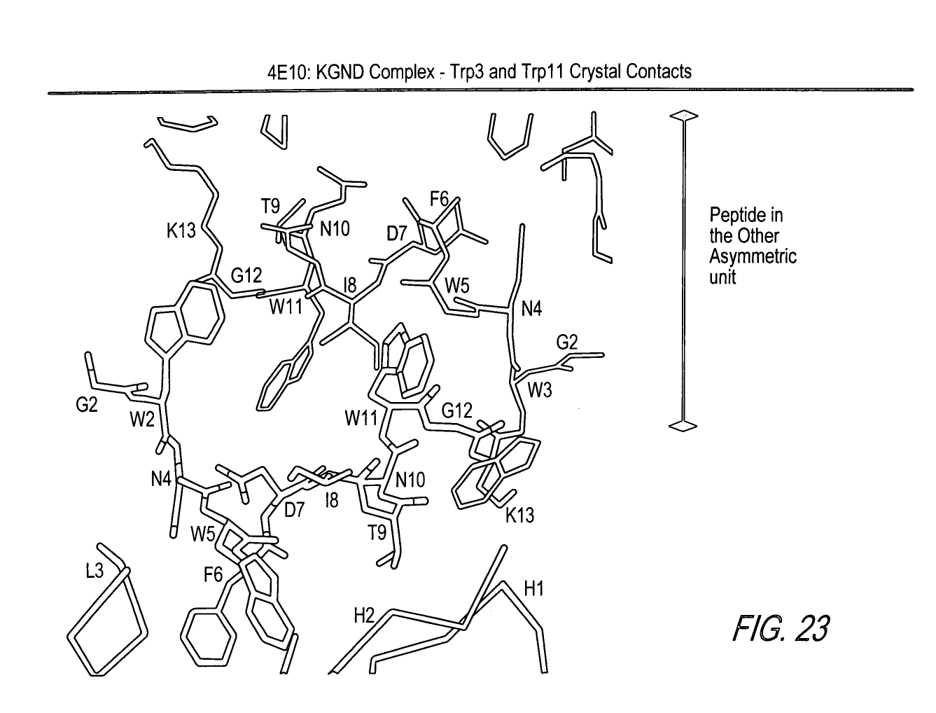

FIG. 24 shows hydrophobic contacts between 4E10 and peptide KGND (residues 2-13 of SEQ ID NO: 2);

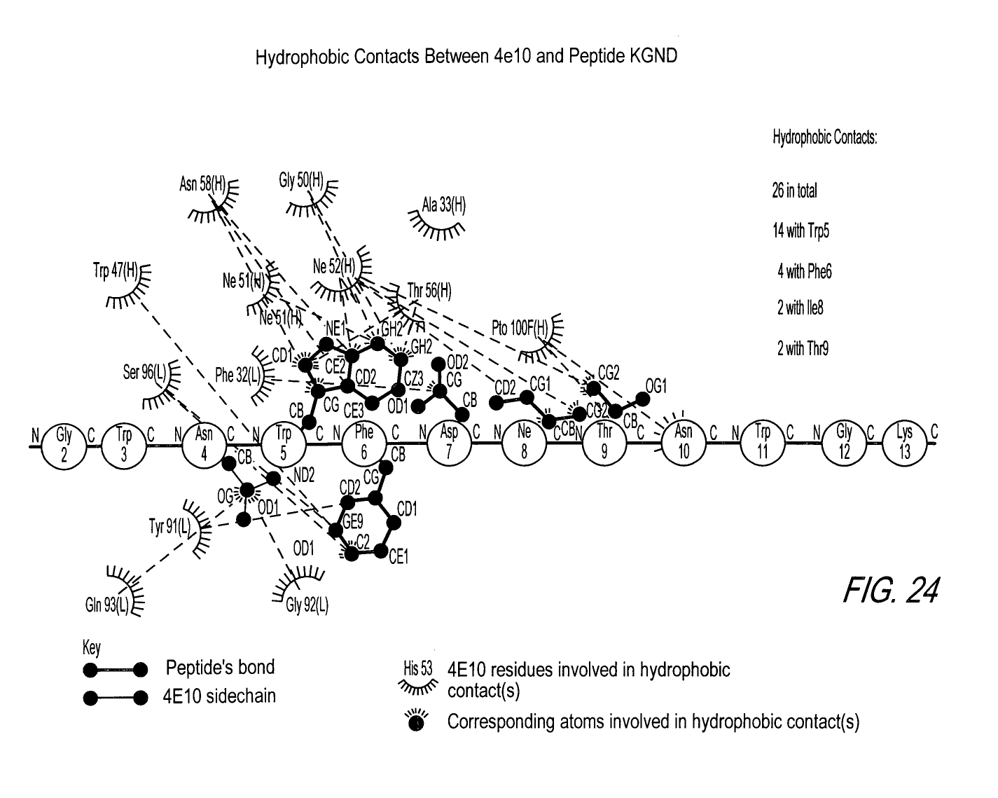

FIG. 25 shows H bonds between 4E10 and peptide KGND (residues 2-13 of SEQ ID NO: 2);

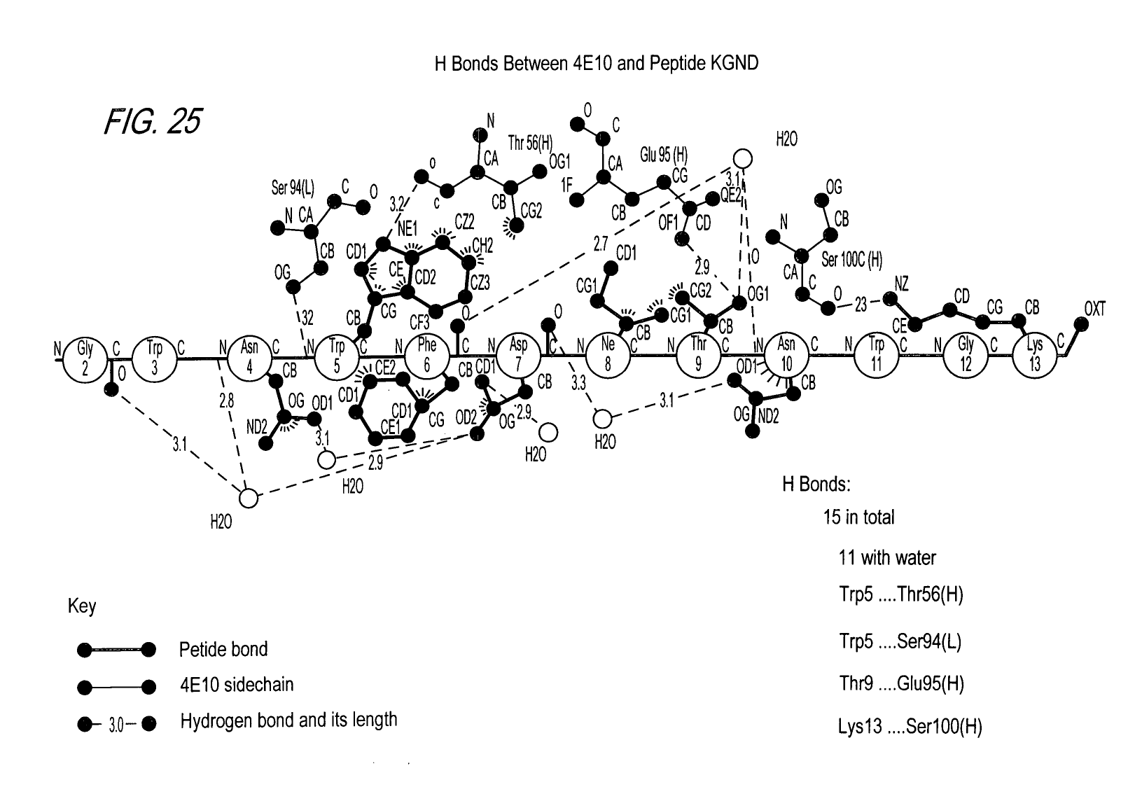

FIG. 26 shows 4E10:KGND complex, Trp5 and Phe6 contacts;

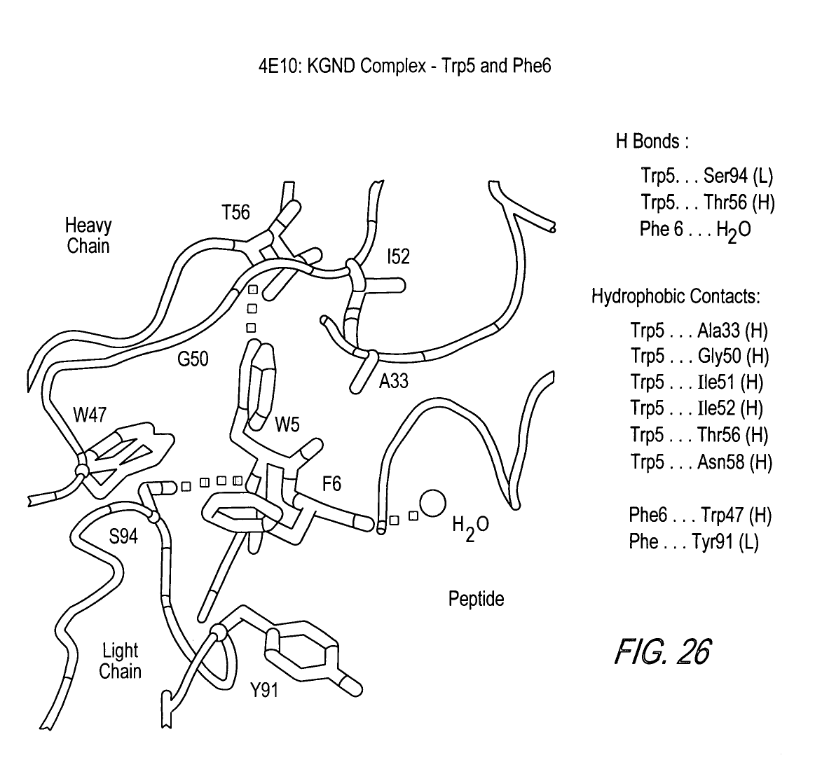

FIG. 27 shows 4E10:KGND complex, Ile8 and Thr9 crystal contacts;

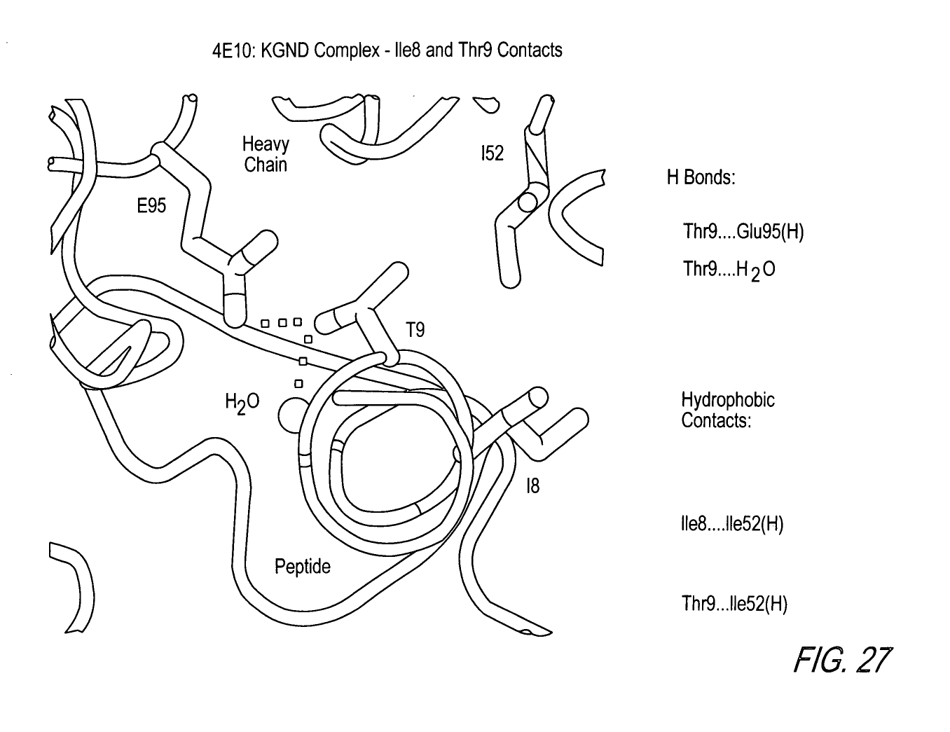

FIG. 28 shows 4E10:peptide KGND crystal packing;

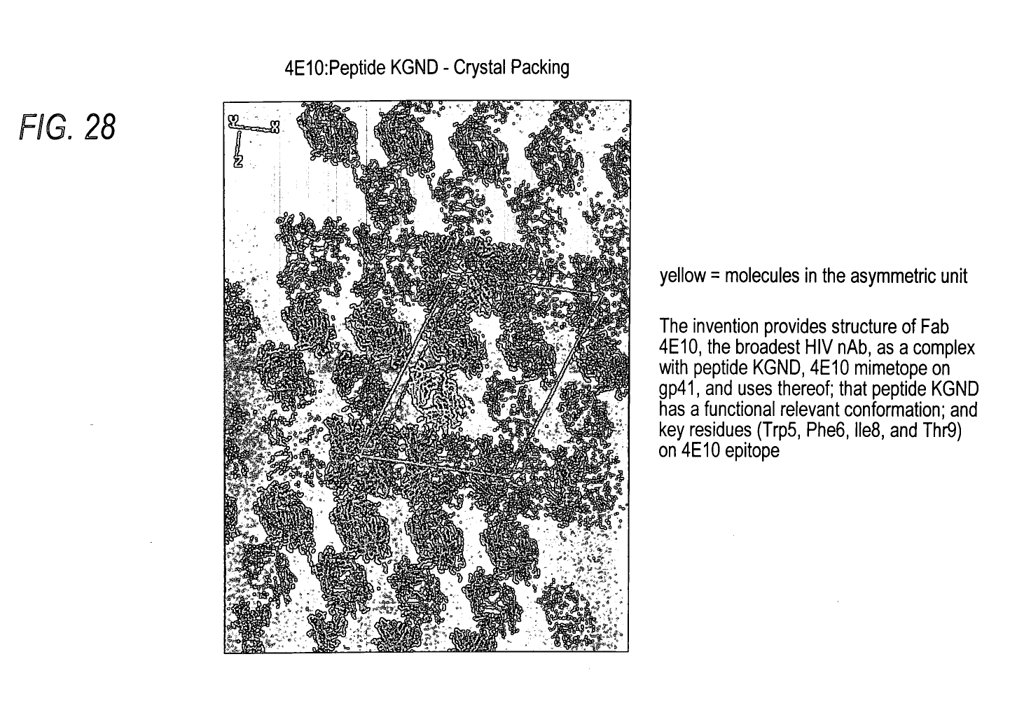

FIG. 29 shows 4E10 vs. b12-Calpha superposition;

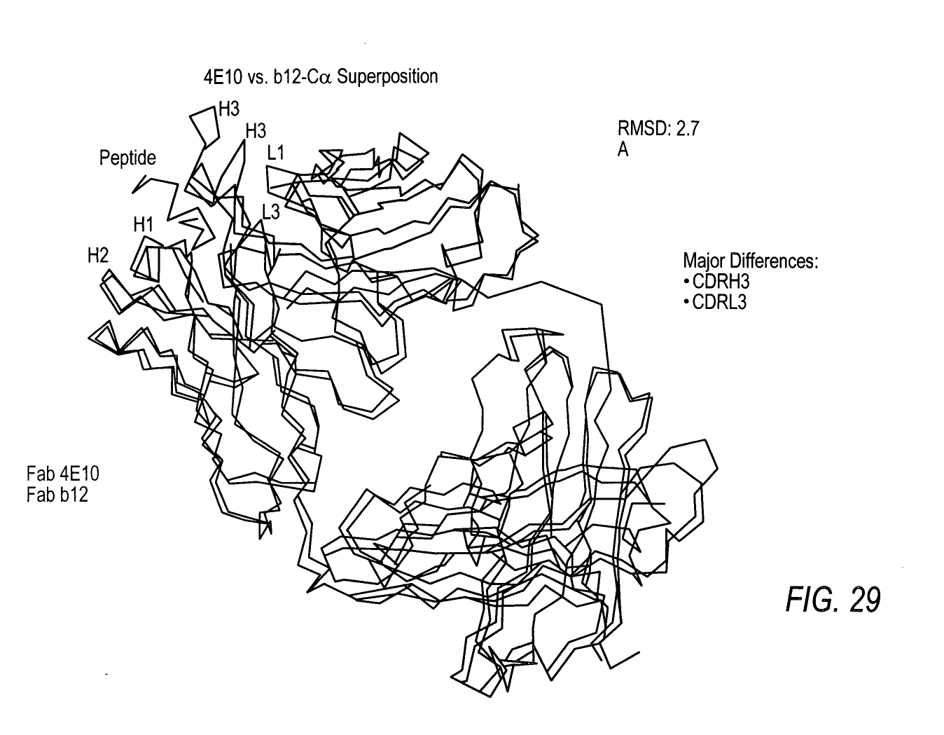

FIG. 30 shows 4E10 vs. b12—CDRH3 and CDRL3;

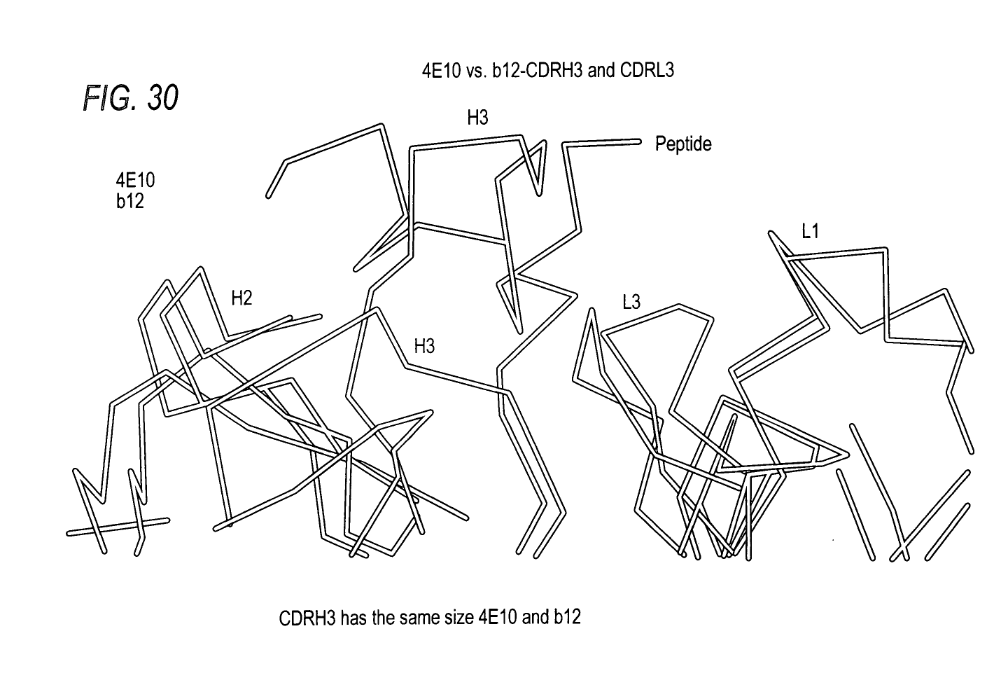

FIG. 31 shows other antibodies complexed with helical peptides;

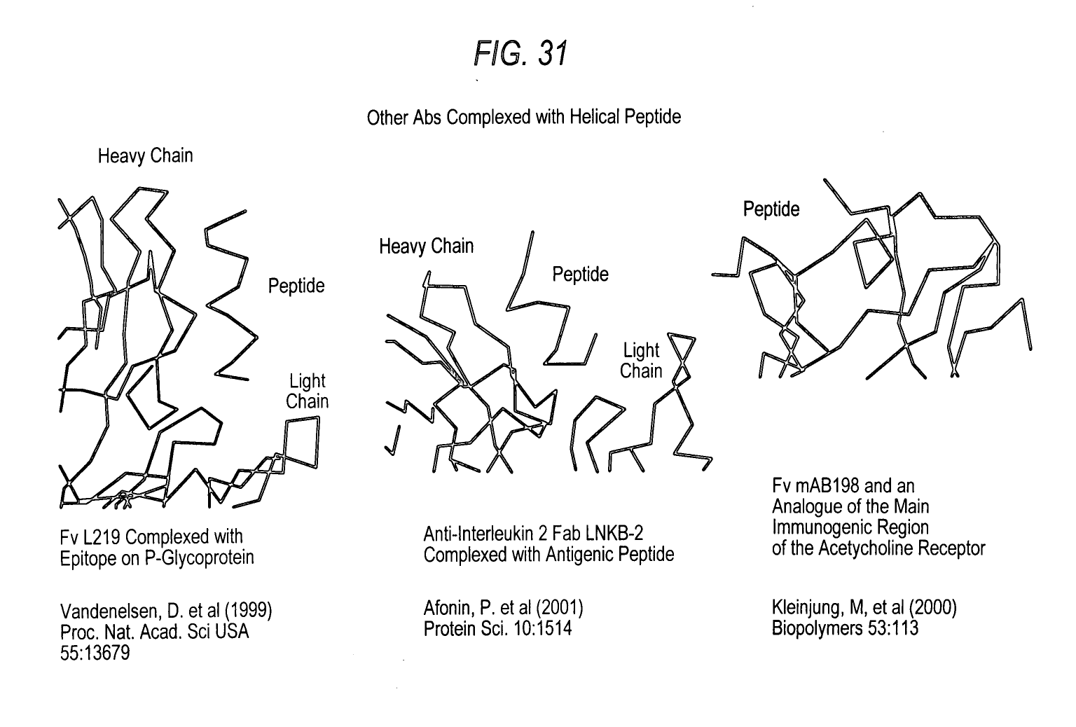

FIG. 32 shows 2F5 complex with its gp41 epitope (SEQ ID NO: 82);

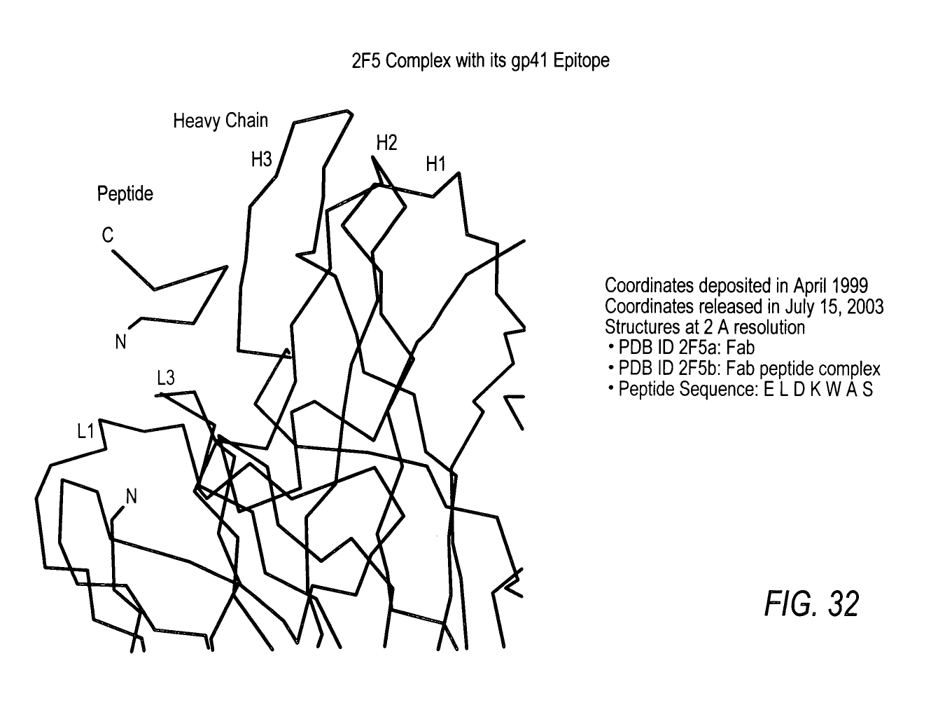

FIG. 33 shows 2F5:epitope complex, epitope configuration (SEQ ID NO: 82);

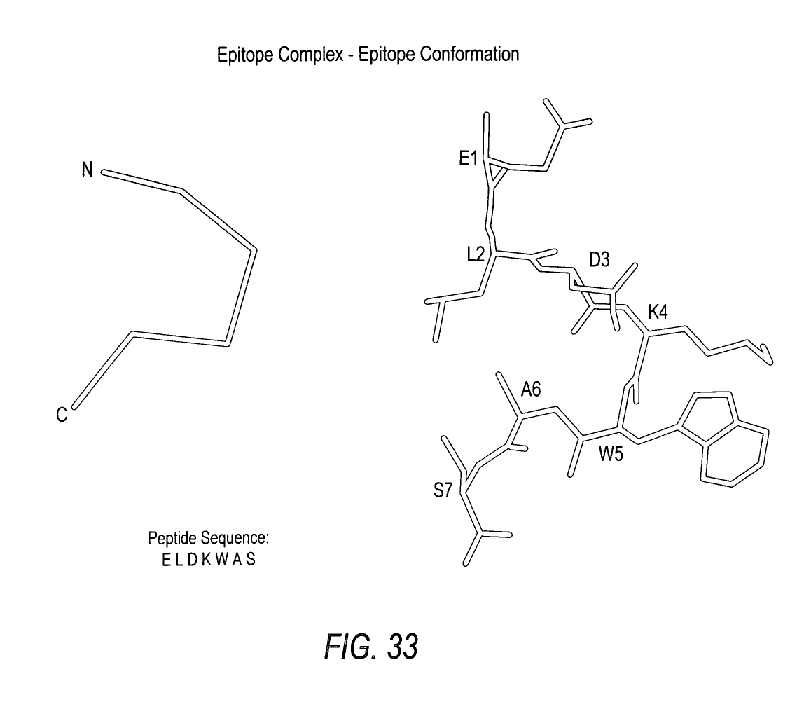

FIG. 34 shows 2F5 as a complex with its epitope (ELDKWAS) (SEQ ID NO: 82);

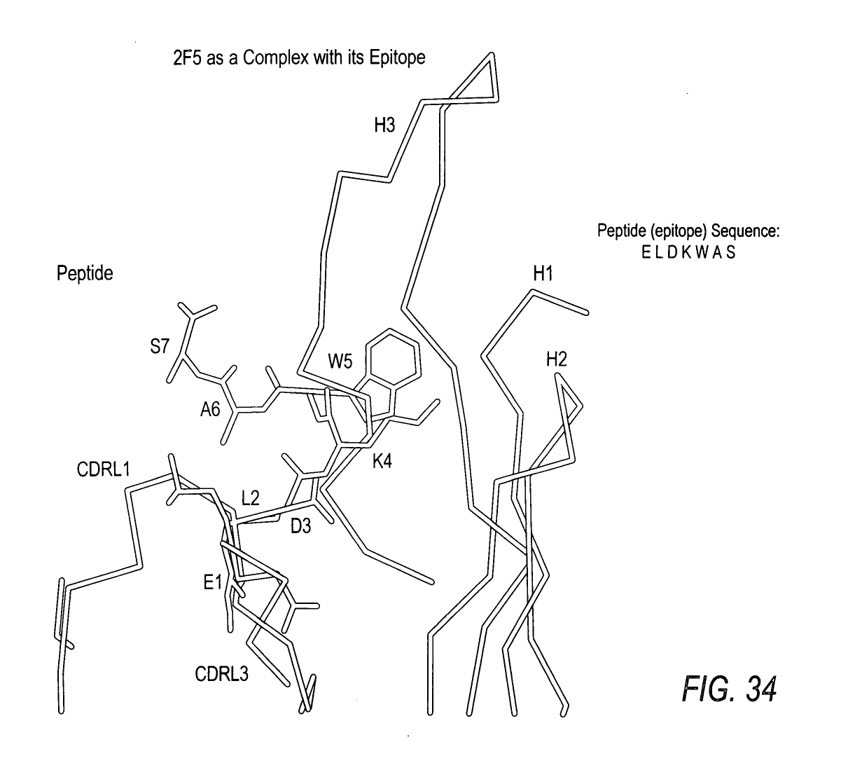

FIG. 35 shows a distribution of key residues in 2F5 and 4E10 epitopes; and

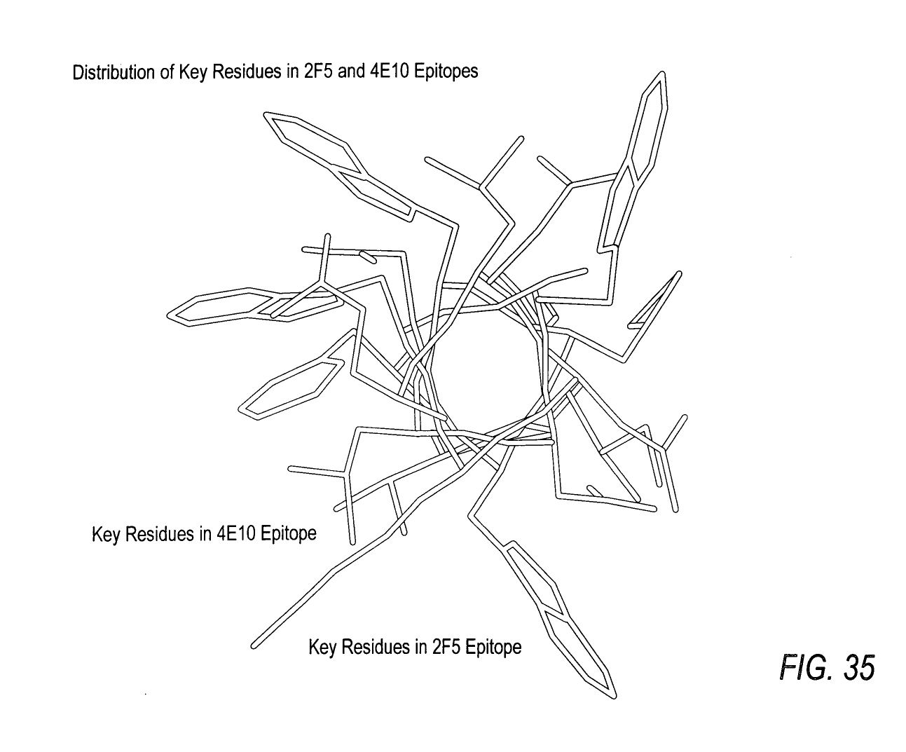

FIG. 36 shows a distribution of key residues in 2F5 and 4E10 epitopes (SEQ ID NO: 93).

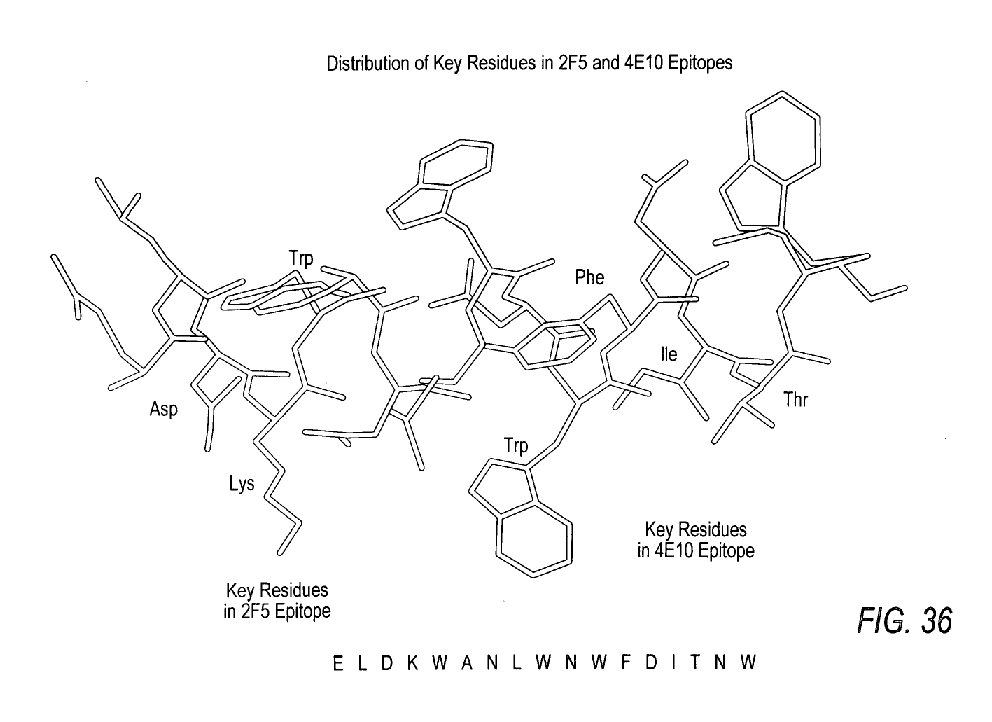

FIG. 37 shows the 4E10 epitope in the context of gp41 and the effect of sequence variation of the epitope on virus neutralization (SEQ ID NOS 94, 83, 95-141 and 80 are disclosed respectively in order of appearance).

FIG. 38 shows the structure of the peptide bound to Fab 4E10.

FIG. 39 shows the antigen binding site of Fab 4E10.

FIG. 40 shows contacts between Fab 4E10 and key residues of its epitope.

FIG. 41 shows a cartoon representation of a hypothetical model of HIV env-mediated membrane fusion and virus neutralization by antibody 4E10.

FIG. 42 shows the structure of gp41 and the current model of HIV gp41. Adapted from Barbato et al., J. Mol. Biol. 2003, 330(5):1101-15.

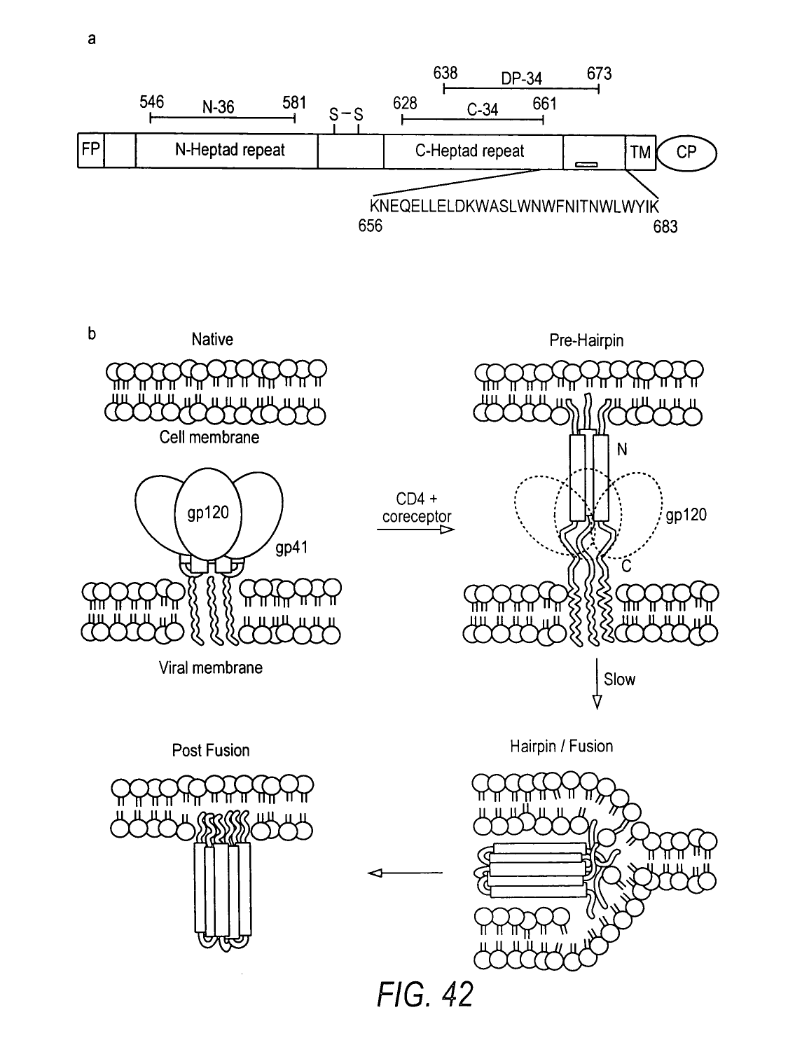

FIG. 43 shows schematic representations of an α-helix with Aib and target cyclic peptides.

FIG. 44 shows the results of competition assays on 44-2 (native sequence) with different peptides: a cycloether (22-4), an Aib-containing peptide (33-1), some lactams (38) and a shorter native sequence.

FIG. 45 shows structures determined for gp41 core peptides (SEQ ID NO: 94).

FIG. 46 shows 4E10 helix small molecule mimetics are synthesized using scaffold that mimics an alpha helix (from Ernst, 2003).

FIG. 47 is a graph depicting the effects of A1a substitutions (along the epitope) on the 4E10 binding to synthetic peptides. The bars represent the ratio log (IC₅₀_{_{—}}peptide reference/IC₅₀_{_{—}}mutant). The values for the log (IC₅₀_{_{—}}peptide reference/IC₅₀_{_{—}}mutant) of W672, F673, T675 represent a minimum, since the IC₅₀ increased by a factor greater than 1,000 when Ala was substituted for those amino acids. IC₅₀ₛ were measured in 2 sets of experiments. For W672, F673, I675, T676, W680, Ala-substitutions were performed on the 14-mer NWFDITNWLWKKKK-NH₂ (SEQ ID NO: 15; IC₅₀=40 nM). For the rest, the substitutions were performed on the 17-mer SLWNWFDITNWLWYIKKKK-NH₂ (SEQ ID NO: 16; IC₅₀=10 nM).

FIG. 48 represents circular dichroism spectra of free 4E10-epitope peptides with or without helix-promiting constraints. The presence of two minima is consistent with a helical conformation. An acyclic compound (in yellow) is compared to its cyclic analog (in blue)(left panel). Native linear sequences of the 4E10 epitope is compared to an Aib-containing analog (right panel).

FIG. 49 is a bar graph showing the ability of peptide NWFDITNWLWYIKKKK-NH₂ (SEQ ID NO: 8) to block neutralization of HIV-1 by 4E10. Replication competent HIV-1 (SF 162 and JR-CSF), produced in human PBMCs, were assayed for neutralization by 4E10 (100 μg/ml) in TZM-b1 cells, in the presence (white bars) or absence (black bars) of an excess of peptide.

FIG. 50 represents graphs showing the effect of peptide NWFDITNWLWYIKKKK-NH₂ (SEQ ID NO: 8) on neutralization of HIV-1 by polyclonal antibodies and sera. HIV-1_{JR-FL}, pseudotyped using the pNL4-3.Luc reporter plasmid, was assayed for neutralization using pooled polyclonal IgG from HIV-1 seropositive individuals (HIVIG), broadly neutralizing serum from the FDA2 individual and normal human serum spiked with 4E10 at 200 μg/ml in the undiluted serum. Neutralization assays were performed using U87.CD4.CCR5 cells as target cells, in the presence (open symbols) or absence (closed symbols) of peptide NWFDITNWLWYIKKKK-NH₂ (SEQ ID NO: 8). Note that the zero point in serum dilution corresponds to only 30 μg/ml 94-1 being present.

FIG. 51 is a helical wheel representation of gp41 (residues 670-682). The key binding residues for 4E10 are shown in red, and are all found on the same side of the helix.

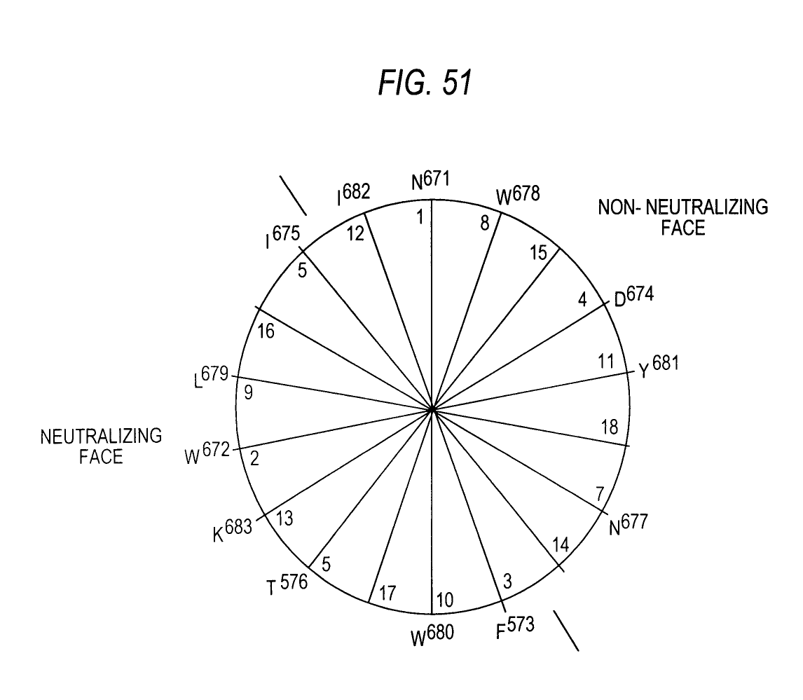

FIG. 52 is a schematic of the vaccine design process where constraints are introduced. The peptides are constrained to a helix conformation via the introduction of an Aib or tether constraint. The “non-neutralizing face” is blocked with the introduction of non-immunogenic bulk so antibody is preferentially elicited against the neutralizing face.

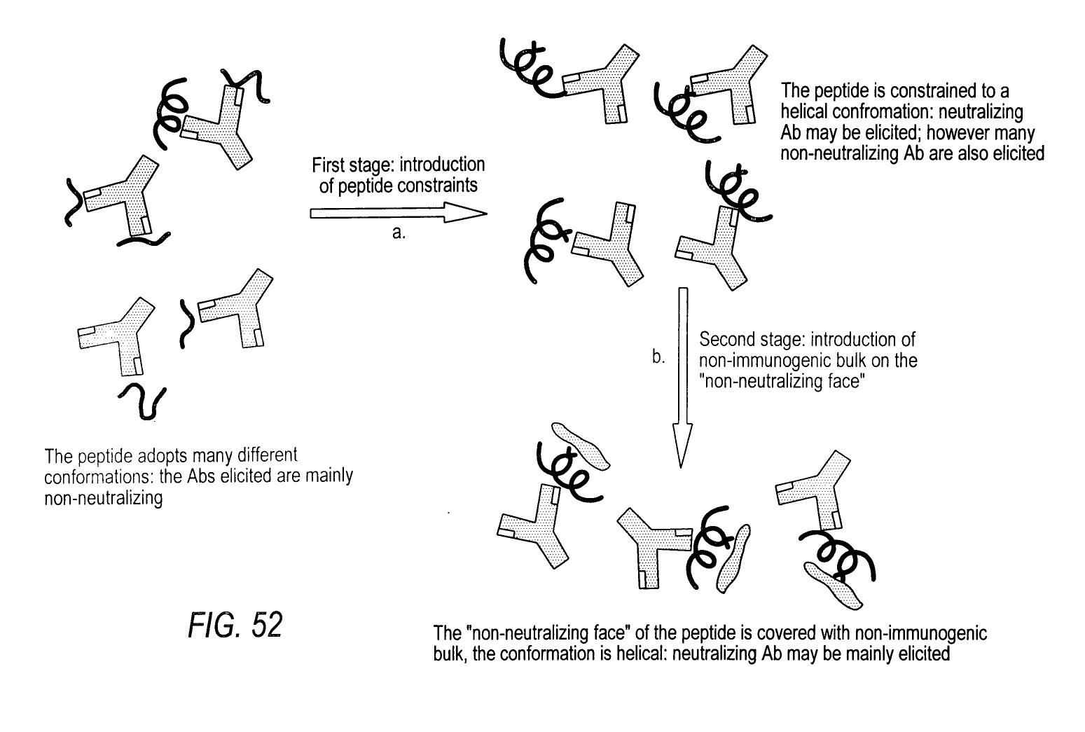

FIG. 53 is a stereo view of the peptide structures superimposed on the sigma A-weighted Fo-Fc electron density omit map contoured at 3.5σ (2.5σ for peptide 94-1). Clear electron density is evident for peptide 104-2 (panel A), peptide 94-1 (panel B) and peptide 33-1 (panel C) residues, except for K683-K686 at the C-terminus of peptide 94-1.

FIG. 54 shows structural similarly among the 4E10-bound peptides. Panels A and B show superposition of the three-dimensional structure of the four peptides (KGND, 94-1, 33-1, and 104-2) bound to Fab 4E10. The structural homology among the peptides is high even in the constrained region of peptides 104-2 and 33-1 as shown in panel B.

FIG. 55 shows contacts between Fab 4E10 and its peptide epitopes. Panel A shows contacts between Fab 4E10 and key epitope residues Trp^{P672}, Phe^{P673}, and Thr^{P676}. The side chains of Trp^{P672} and Phe^{P673} are involved in aromatic π-stacking interactions with 4E10 residues Tyr^{L91}, Trp^{H47}, and Phe^{H100J}. Panel B shows contacts between epitope residue TrpP680 and CDR H3 of 4E10. Tyr⁶⁸¹ could help to stabilize Trp^{P680} in an optimal conformation for interaction with the antibody. Panel C shows a cluster of Ile/Leu made at the combining site using CDR H2 residues and epitope residues Ile⁶⁷⁵, Leu⁶⁷⁹, and Ile⁶⁸².

### DETAILED DESCRIPTION OF THE INVENTION

As discussed herein and illustrated in the Figures, the invention pertains to the structure of Fab 4E10, e.g., as a complex with herein identified peptide KGND, herein described as a 4E10 mimetope on gp41, as determined by crystallographic techniques, and to the confirmation that peptide KGND has a functional relevant conformation, as well as to the determination of key residues on 4E10. As likewise discussed herein, the present invention thus provides a means for identifying or designing compounds, such as peptides or derivatized peptides (e.g., N-acylated or N-alkylated peptides, wherein carbon chains advantageously have up to 12, e.g., up to 6 carbons, and may be substituted, e.g., with one or more hetero-atoms such as N, S, or O), that bind to the antibody. Similarly, the present invention also provides a means for identifying or designing compounds that bind to the KGND binding domain in the antibody. The design of these compounds that act as an immunogen is based on the crystal structure described herein. These compounds, when administered, elicit anti-HIV antibodies. The compounds may then be used in diagnostic, pharmaceutical, immunogenic, immunological or vaccine compositions. These compositions are useful in the detection or treatment and/or prevention of HIV infections. And, antibodies elicited by such compounds also can be used in diagnostic or pharmaceutical, immunogenic, immunological or vaccine compositions.

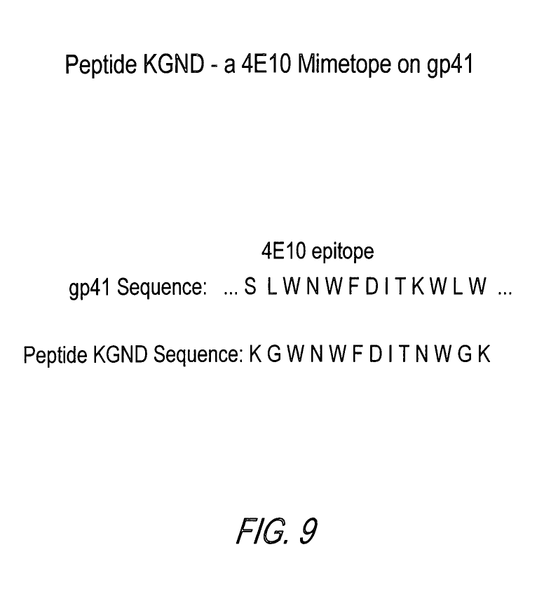

Additionally, the invention pertains to the identification, design, synthesis and isolation of the polypeptide herein referred to as KGND, which has the sequence set forth in FIG. 9. The present invention also relates to homologues, derivatives and variants of KGND, wherein it is preferred that the homologue, derivative or variant have at least 50%, at least 60%, at least 70%, and least 75%, at least 80%, at least 85%, at least 90%, at least 93%, at least 95%, at least 97%, at least 98% or at least 99% homology or identity with the sequence of KGND. It is noted that within this specification, homology to KGND refers to the homology of the homologue, derivative or variant to the binding site of KGND. In this respect, when determining the percent homology of a compound that consisted essentially of a non-peptidic backbone containing side chains that were homologues, derivatives or variants of KGND, only the composition of the side chain would be used in determining the percent homology; the percent homology is determined solely for the portion of the compound which contains the equivalent of KGND's binding site.

Yet further still, the invention relates to the conformational structure of KGND, as described herein. Furthermore, it is assumed that any homologues, derivatives and variants of KGND would encompass the conformational structure of KGND as described herein.

The invention still further relates to nucleic acid sequences expressing KGND, or homologues, variants or derivatives thereof. One of skill in the art will know, recognize and understand techniques used to create such. Additionally, one of skill in the art will be able to incorporate such a nucleic acid sequence into an appropriate vector, allowing for production of the amino acid sequence of KGND or a homologue, variant or derivative thereof.

Where used herein and unless specifically indicated otherwise, the following terms are intended to have the following meanings in addition to any broader (or narrower) meanings the terms might enjoy in the art:

The term “isolated” is used herein to indicate that the isolated moiety (e.g. peptide or compound) exists in a physical milieu distinct from that in which it occurs in nature. For example, the isolated peptide may be substantially isolated with respect to the complex cellular milieu in which it naturally occurs. The absolute level of purity is not critical, and those skilled in the art can readily determine appropriate levels of purity according to the use to which the peptide is to be put. The term “isolating” when used a step in a process is to be interpreted accordingly.

In many circumstances, the isolated moiety will form part of a composition (for example a more or less crude extract containing many other molecules and substances), buffer system, matrix or excipient, which may for example contain other components (including proteins, such as albumin).

In other circumstances, the isolated moiety may be purified to essential homogeneity, for example as determined by PAGE or column chromatography (for example HPLC or mass spectrometry). In preferred embodiments, the isolated peptide or nucleic acid of the invention is essentially the sole peptide or nucleic acid in a given composition.

The proteins and compounds of the invention need not be isolated in the sense defined above, however.

The term “pharmaceutical composition” is used herein to define a solid or liquid composition in a form, concentration and level of purity suitable for administration to a patient (e.g. a human patient) upon which administration it can elicit the desired physiological changes. The terms “immunogenic composition” and “immunological composition” and “immunogenic or immunological composition” cover any composition that elicits an immune response against the targeted pathogen, HIV. Terms such as “vaccinal composition” and “vaccine” and “vaccine composition” cover any composition that induces a protective immune response against the targeted pathogen or which efficaciously protects against the pathogen; for instance, after administration or injection, elicits a protective immune response against the targeted pathogen or provides efficacious protection against the pathogen. Accordingly, an immunogenic or immunological composition induces an immune response which can, but need not, be a protective immune response. An immunogenic or immunological composition can be used in the treatment of individuals infected with the pathogen, e.g., to stimulate an immune response against the pathogen, such as by stimulating antibodies against the pathogen. Thus, an immunogenic or immunological composition can be a pharmaceutical composition. Furthermore, when the text speaks of “immunogen, antigen or epitope”, an immunogen can be an antigen or an epitope of an antigen. A diagnostic composition is a composition containing a compound or antibody, e.g., a labeled compound or antibody, that is used for detecting the presence in a sample, such as a biological sample, e.g., blood, semen, vaginal fluid, etc, of an antibody that binds to the compound or an immunogen, antigen or epitope that binds to the antibody; for instance, an anti-HIV antibody or an HIV immunogen, antigen or epitope.

A “binding site” can be a site (such as an atom, a functional group of an amino acid residue or a plurality of such atoms and/or groups) in a binding cavity or region, which may bind to a compound such as a candidate immunogen, antigen or epitope, protein, peptide, derivatized protein or peptide, or compound. An “active site” can be a site (such as an atom, a functional group of an amino acid residue or a plurality of such atoms and/or groups) in a binding cavity or region, which is/are involved in binding.

By “fitting”, is meant determining by automatic, or semi-automatic means, interactions between one or more atoms of a candidate molecule and at least one atom of a structure of the invention, and calculating the extent to which such interactions are stable. Interactions include attraction and repulsion, brought about by charge, steric considerations and the like. Various computer-based methods for fitting are described further herein.

By “helix” or “helical”, is meant a helix as known in the art, including, but not limited to an alpha-helix. Additionally, the term helix or helical may also be used to indicate a c-terminal helical element with an N-terminal turn.

By “root mean square (or rms) deviation”, we mean the square root of the arithmetic mean of the squares of the deviations from the mean.

By a “computer system”, we mean the hardware means, software means and data storage means used to analyse atomic coordinate data. The minimum hardware means of the computer-based systems of the present invention typically comprises a central processing unit (CPU), input means, output means and data storage means. Desirably a monitor is provided to visualize structure data. The data storage means may be RAM or means for accessing computer readable media of the invention. Examples of such systems are microcomputer workstations available from Silicon Graphics Incorporated and Sun Microsystems running Unix based, Windows NT or IBM OS/2 operating systems.

By “computer readable media”, we mean any medium or media, which can be read and accessed directly by a computer e.g. so that the media is suitable for use in the above-mentioned computer system. Such media include, but are not limited to: magnetic storage media such as floppy discs, hard disc storage medium and magnetic tape; optical storage media such as optical discs or CD-ROM; electrical storage media such as RAM and ROM; and hybrids of these categories such as magnetic/optical storage media.

A “conservative amino acid change” is one in which the amino acid residue is replaced with an amino acid residue having a similar side chain. Families of amino acid residues having similar side chains have been defined in the art. These families include amino acids with basic side chains (e.g. lysine, arginine and histidine), acidic side chains (e.g. aspartic acid and glutamic acid), non-charged amino acids or polar side chains (e.g. glycine, asparagine, glutamine, serine, threonine, tyrosine and cysteine), non-polar side chains (e.g. alanine, valine, leucine, isoleucine, proline, phenylalanine, methionine and tryptophan), beta-branched side chains (e.g. threonine, valine and isoleucine), and aromatic side chains (e.g. tyrosine, phenylalanine, tryptophan and histidine).

Conservative substitutions may be made to relevant amino acid sequences of interest in accordance with the following chart:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Thus, references herein to proteins and peptides that are to some defined extent “identical” (or which share a defined extent of “identity”) with a reference protein or peptide may also optionally be interpreted to include proteins and peptides in which conservative amino acid changes are disregarded so that the original amino acid and its changed counterpart are regarded as identical for the purposes of sequence comparisons. Accordingly, the invention can comprehend proteins or peptides and the use thereof having conservative amino acid changes as to KGND, so long as the three dimensional structure, as defined herein, is maintained, e.g., so that there is binding/complexing with Fab 4E10.

For the purposes of the present invention, sequence identity or homology is determined by comparing the amino acid sequences of the proteins when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin and Altschul (1990) Proc. Natl. Acad. Sci. USA 87: 2264-2268, modified as in Karlin and Altschul (1993) Proc. Natl. Acad. Sci. USA 90: 5873-5877.

Another example of a mathematical algorithm used for comparison of sequences is the algorithm of Myers and Miller (1988) CABIOS 4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85: 2444-2448.

Preferred for use according to the present invention is the WU-BLAST (Washington University BLAST) version 2.0 software. WU-BLAST version 2.0 executable programs for several UNIX platforms can be obtained from Washington University. This program is based on WU-BLAST version 1.4, which in turn is based on the public domain NCBI-BLAST version 1.4 (Altschul and Gish, 1996; Altschul et al., 1990; Gish and States, 1993; Karlin and Altschul, 1993; all of which are incorporated by reference herein).

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC(N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur and Lipman, 1983, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences.

And, without undue experimentation, the skilled artisan can consult with many other programs or references for determining percent homology.

The synthetic KGND polypeptide described herein may be chemically synthesized in whole or part using techniques that are well-known in the art (see, e.g., Kochendoerfer, G. G., 2001). Additionally, homologs and derivatives of the polypeptide may be also be synthesized.

Alternatively, methods which are well known to those skilled in the art can be used to construct expression vectors containing nucleic acid molecules that encode the polypeptide or homologs or derivatives thereof under appropriate transcriptional/translational control signals, for expression. These methods include in vitro recombinant DNA techniques, synthetic techniques and in vivo recombination/genetic recombination. See, for example, the techniques described in Maniatis et al., 1989. The Fab 4E10 antibody is obtained as described herein and in the literature.

The crystals of the invention can be obtained by conventional means as are well-known in the art of protein crystallography, including batch, liquid bridge, dialysis, vapor diffusion and hanging drop methods (see, e.g., McPherson, 1982; McPherson, 1990; Webber, 1991). Generally, the crystals of the invention are grown by dissolving substantially pure Fab 4E10 and compound (e.g., polypeptide KGND in example, but other compounds may be used to test if such compounds form crystals analogous to those disclosed herein) in an aqueous buffer containing a precipitant at a concentration just below that necessary to precipitate the protein. Water is removed by controlled evaporation to produce precipitating conditions, which are maintained until crystal growth ceases.

The crystals of the invention, and particularly the atomic structure co-ordinates obtained therefrom, have a wide variety of uses. The crystals and structure co-ordinates are particularly useful for identifying compounds that bind to Fab 4E10 and thus are useful to elicit anti-HIV antibodies. Such compounds are useful in eliciting clade B anti-HIV antibodies, however variants may be useful in eliciting clade A, C, D or E anti-HIV antibodies.

The structure co-ordinates described herein can be used as phasing models in determining the crystal structures of additional synthetic or mutated Fab, 4 E10 domains, as well as the structures of co-crystals of such domains with ligands.

The provision of the crystal structure of Fab 4E10 complexed with KGND in Table 1 and the Figures provide the skilled artisan with a detailed insight into the mechanisms of action of Fab 4E10. This insight provides a means to design compounds that bind to Fab 4E10 and thus to certain anti-HIV antibodies, and therefore compounds that elicit anti-HIV antibodies, which are useful in diagnosis, treatment, or prevention of HIV in an individual in need thereof.

The provision of the crystal structure of Fab 4E10 complexed with KGND allows a novel approach for drug or compound discovery, identification, and design for compounds that bind to to Fab 4E10 and thus to anti-HIV antibodies, and therefore compounds that elicit anti-HIV antibodies, which are useful in diagnosis, treatment, or prevention of HIV in an individual in need thereof. Accordingly, the invention provides a computer-based method of rational drug or compound design or identification which comprises: providing the structure of the Fab 4E10 complex as defined by the co-ordinates or the identifying co-ordinates in Table 1 and/or in the Figures; providing a structure of a candidate compound; and fitting the structure of the candidate to the structure of Fab 4E10 of Table 1 and the Figures.

In an alternative aspect, the method may use the co-ordinates of atoms of interest of Fab 4E10 which are in the vicinity of the active site or binding region in order to model the pocket in which the substrate or ligand binds. These co-ordinates may be used to define a space which is then screened “in silico” against a candidate molecule. Thus, the invention provides a computer-based method of rational drug or compound design or identification which comprises: providing the co-ordinates of at least two atoms of Table 1 (“selected co-ordinates”); providing the structure of a candidate compound; and fitting the structure of the candidate to the selected coordinates.

In practice, it may be desirable to model a sufficient number of atoms of Fab 4E10 as defined by the co-ordinates of Table 1 which represent the active site or binding region. Thus, there can be provided the co-ordinates of at least 5, advantageously at least 10, more advantageously at least 50 and even more advantageously at least 100 atoms of the structure. Accordingly, the methods of the invention can employ a sub-domain of interest of Fab 4E10 which is in the vicinity of the active site or binding region, and the invention can provide a computer-based method for identifying or rationally designing a compound or drug which comprises: providing the co-ordinates of at least a sub-domain of; providing the structure of a candidate modulator or inhibitor of Fab 4E10; and fitting the structure of the candidate to the coordinates of the Fab 4E10 sub-domain provided.

These methods can optionally include synthesizing the candidate and can optionally further include contacting the candidate with Fab 4E10 to test whether there is binding and/or inhibition and/or administering the compound to an animal capable of eliciting antibodies and testing whether the compound elicits anti-HIV antibodies. Compounds which elicit anti-HIV antibodies are useful for diagnostic purposes, as well as for immunogenic, immunological or even vaccine compositions, as well as pharmaceutical compositions.

“Fitting” can mean determining, by automatic or semi-automatic means, interactions between at least one atom of the candidate and at least one atom of Fab 4E10 and calculating the extent to which such an interaction is stable. Interactions can include attraction, repulsion, brought about by charge, steric considerations, and the like. A “sub-domain” can mean at least one, e.g., one, two, three, or four, complete element(s) of secondary structure. Particular regions of Fab 4E10 include those identified in Table 1.

The step of providing the structure of a candidate molecule may involve selecting the compound by computationally screening a database of compounds for interaction with the active site. For example, a 3-D descriptor for the potential modulator may be derived, the descriptor including geometric and functional constraints derived from the architecture and chemical nature of the active site. The descriptor may then be used to interrogate the compound database, a potential modulator being a compound that has a good match to the features of the descriptor. In effect, the descriptor can be a type of virtual pharmacophore.

In any event, the determination of the three-dimensional structure of Fab 4E10 complex provides a basis for the design of new and specific compounds that bind to Fab 4E10 and are useful for eliciting an immune response. For example, from knowing the three-dimensional structure of Fab 4E10 complex, computer modelling programs may be used to design or identify different molecules expected to interact with possible or confirmed active sites such as binding sites or other structural or functional features of Fab 4E10. More specifically, a compound that potentially binds (“binder”) to Fab 4E10 activity can be examined through the use of computer modeling using a docking program such as GRAM, DOCK or AUTODOCK (see Walters et al. Drug Discovery Today, vol. 3, no. 4 (1998), 160-178, and Dunbrack et al. Folding and Design 2 (1997), 27-42). This procedure can include computer fitting of potential binders to FAB 4E10 to ascertain how well the shape and the chemical structure of the potential binder will bind to the antibody.

Also, computer-assisted, manual examination of the active site or binding site of Fab 4E10 may be performed. The use of programs such as GRID (P. Goodford, J. Med. Chem., 1985, 28, 849-57)—program that determines probable interaction sites between molecules with various functional groups and the antibody—may also be used to analyze the active site or binding site to predict partial structures of binding compounds.

Computer programs can be employed to estimate the attraction, repulsion or steric hindrance of the two binding partners, e.g., Fab 4E10 and a candidate binder. Generally, the tighter the fit, the fewer the steric hindrances, and the greater the attractive forces, the more potent the potential binder, since these properties are consistent with a tighter binding constant. Furthermore, the more specificity in the design of a candidate binder, the more likely it is that it will not interact with other proteins as well.

In a further aspect, the invention provides for a method for determining the structure of a binder of Fab 4E10 bound to Fab 4E10, said method comprising, (a) providing a crystal of Fab 4E10 according to the invention, (b) soaking the crystal or another crystal with said binder; and (c) determining the structure of said Fab 4E10-binder complex. Such other crystal may have essentially the same coordinates discussed herein, however due to minor alterations in the polypeptide or sequence, the crystal may form in a different space group.

The invention further involves, in place of or in addition to in silico methods, high throughput screening of compounds to select compounds with binding activity. Those compounds which show binding activity may be selected as possible candidate binders, and further crystallized with Fab 4E10, e.g., by co-crystallization or by soaking, for X-ray analysis. The resulting X-ray structure may be compared with that of Table 1 and the information in the Figures for a variety of purposes. For example, where the contacts made by such compounds overlap with those made by Fab 4E10, novel molecules comprising residues which contain contacts of Fab 4E10 and other compounds may be provided. Compounds of the present invention may comprise or consist essentially of polypeptides having a sequence consisting essentially of DKWX¹X²X³X⁴X⁵WFXIT (SEQ ID NO: 3), wherein X is N, D, S, or G, X¹=A or a conservative substitution thereof, X²=N or a conservative substitution thereof, X³=L or a conservative substitution thereof, X⁴=W or a conservative substitution thereof, X⁵=N, S or T or a conservative substitution thereof, wherein the polypeptide has a helical structure, and it is not otherwise disclosed in he art. Furthermore, said compounds may also comprise or consist essentially of a polypeptide having a sequence consisting essentially of DKWX¹X²X³X⁴X⁵WFXIT (SEQ ID NO: 3), wherein X=N, D, S, G, Q, C, T, M, E, K, R, A, P, I, L, V, O, Aib, or other natural or synthetic amino acids, including conservative substitutions thereof, X¹=A, G, P, I, L, V, Aib, or other natural or synthetic amino acids, or a conservative substitution thereof; X²=N, Q, C, S, T, M, or other natural or synthetic amino acids, or a conservative substitution thereof; X³=L, I, V, G, A, P, or other natural or synthetic amino acids, or a conservative substitution thereof, X⁴=W, H, F, Y, K, C, Aib, or other natural or synthetic amino acids, or a conservative substitution thereof, X⁵=N, S, T, Q, C, M, E, A, or other natural or synthetic amino acids, or a conservative substitution thereof; wherein the polypeptide has a helical structure, and it is not otherwise disclosed in the art. In one embodiment, these polypeptides may include Aib inserted between any two amino acids of WFXIT. In another embodiment, the polypeptides may be branched, including wherein WFXIT is branched. It is an aspect of the present invention that any branched chains may be sufficiently short in length, or circular or helical in structure such that the peptide is able to bind to Fab 4E10. In yet another aspect of the invention, the polypeptide comprises or consists essentially of a peptide as shown in Table 4.

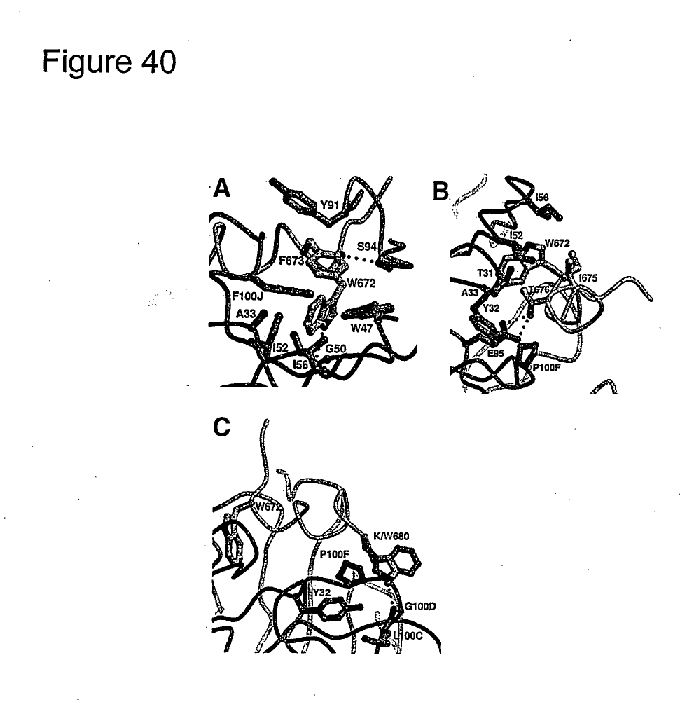

In yet another aspect, the invention also encompasses a polypeptide having a sequence consisting essentially of DKWX¹X²X³X⁴X⁵WFXITXX⁶XW (SEQ ID NO: 4), wherein X=N, D, S, G, Q, C, T, M, E, K, R, A, P, I, L, V, O, Aib, or other natural or synthetic amino acids, including conservative substitutions thereof, X¹=A, G, P, I, L, V, Aib, or other natural or synthetic amino acids, or a conservative substitution thereof; X²=N, Q, C, S, T, M, or other natural or synthetic amino acids, or a conservative substitution thereof; X³=L, I, V, G, A, P, or other natural or synthetic amino acids, or a conservative substitution thereof, X⁴=W, H, F, Y, K, C, Aib, or other natural or synthetic amino acids, or a conservative substitution thereof, X⁵=N, S, T, Q, C, M, E, A, or other natural or synthetic amino acids, or a conservative substitution thereof, X⁶=any natural or synthetic amino acids; and wherein the polypeptide has a helical structure. In one embodiment, the peptide binds to Fab 4E10. In one embodiment, X⁶ is W, such that the polypeptide has the sequence consisting essentially of DKWX¹X²X³X⁴X⁵WFXITXWXW (SEQ ID NO: 5), wherein the sequence includes an additional two tryptophans, as depicted in FIG. 40C.

The invention also encompasses a polypeptide having a sequence consisting essentially of XNWFX¹ITX²WLWX (SEQ ID NO: 6), wherein X comprises 0-8 amino acids consisting essentially of K, Aib, Y, I, or other natural or synthetic amino acids, including conservative substitutions thereof; wherein X¹=D, C, or other natural or synthetic amino acids, including conservative substitutions thereof; wherein X²=0, N, or other natural or synthetic amino acids or a conservative substitution thereof, wherein the polypeptide has a helical structure, and is not otherwise disclosed in the art. In one embodiment, Aib may be inserted between any two amino acids of WFX¹IT (SEQ ID NO: 79). Alternatively or additionally, WFX¹ IT (SEQ ID NO: 79) can be branched. The branched chain can be of sufficient length and/or configuration that the polypeptide binds to Fab 4E10. In another embodiment, the polypeptide comprises or consists essentially of: NWFCITOWLWKKKK-NH₂ (SEQ ID NO: 7); NWFDITNWLWYIKKKK-NH₂ (SEQ ID NO: 8); NWFDITNWLWK-Aib-K-Aib-K-NH₂ (SEQ ID NO: 9); KK-Aib-NWFDITNWLWK-Aib-K-Aib-K-NH₂ (SEQ ID NO: 10); NWFDITYNWLWYIK-Aib-K-Aib-KK-NH₂ (SEQ ID NO: 11); or NWFCITOWLWKKKK-NH₂ (SEQ ID NO: 12).

The invention additionally encompasses a polypeptide having a sequence consisting essentially of: NWFX¹ITX²WLWX (SEQ ID NO: 13), wherein X comprises 0-8 amino acids consisting essentially of K, Aib, Y, I, or other natural or synthetic amino acids, including conservative substitutions thereof; wherein X^{I}=D, C, or other natural or synthetic amino acids or a conservative substitution thereof; wherein X²=0, N, or other natural or synthetic amino acids or a conservative substitution thereof; wherein the polypeptide has a helical structure, and is not otherwise disclosed in the art.

Having designed, identified, or selected possible binding candidate binders by determining those which have favorable fitting properties, e.g., strong attraction between a candidate and Fab 4E10, these can then be screened for activity. Consequently, the invention further involves: obtaining or synthesizing the candidate modulator or inhibitor; and contacting the candidate binder with Fab 4E10 to determine the ability of the candidate to bind with Fab 4E10. In the latter step, the candidate is advantageously contacted with Fab 4E10 under conditions to determine its function. Instead of, or in addition to, performing such an assay, the invention may comprise: obtaining or synthesizing the candidate modulator, forming a complex of Fab 4E10 and the candidate, and analyzing the complex, e.g., by X-ray diffraction or NMR or other means, to determine the ability of the candidate to interact with Fab 4E10. Detailed structural information can then be obtained about the binding of the candidate to Fab 4E10, and in light of this information, adjustments can be made to the structure or functionality of the potential modulator, e.g., to improve its binding to Fab 4E10. These steps may be repeated and re-repeated as necessary. Alternatively or additionally, potential binders can be administered to an animal capable of eliciting an antibody response, to ascertain whether the potential binder elicits anti-HIV antibodies.

The invention further involves a method of determining three dimensional structures of Fab 4E10 and KGND homologues of unknown structure by using the structural co-ordinates of Table 1 and the information in the Figures. For example, if X-ray crystallographic or NMR spectroscopic data are provided for a Fab 4E10 and/or KGND homologue of unknown structure, the structure of Fab 4E10 complex as defined in Table 1 and the Figures may be used to interpret that data to provide a likely structure for the Fab 4E10 and/or KGND homologue by techniques well known in the art, e.g., by phase modeling in the case of X-ray crystallography. Thus, an inventive method can comprise: aligning a representation of an amino acid sequence of a Fab 4E10 and/or KGND homologue of unknown structure with the amino acid sequence of Fab 4E10 and/or KGND to match homologous regions of the amino acid sequences; modeling the structure of the matched homologous regions of the Fab 4E10 and/or KGND of unknown structure on the structure as defined in Table 1 and/or in the Figures of the corresponding regions of Fab 4E10 and/or KGND; and, determining a conformation (e.g. so that favorable interactions are formed within the Fab 4E10 and/or KGND of unknown structure and/or so that a low energy conformation is formed) for the Fab 4E10 and/or KGND of unknown structure which substantially preserves the structure of said matched homologous regions. “Homologous regions” describes amino acid residues in two sequences that are identical or have similar, e.g., aliphatic, aromatic, polar, negatively charged, or positively charged, side-chain chemical groups. Identical and similar residues in homologous regions are sometimes described as being respectively “invariant” and “conserved” by those skilled in the art. Advantageously, the first and third steps are performed by computer modeling. Homology modeling is a technique that is well known to those skilled in the art (see, e.g., Greer, 1985; and Blundell et al. 1988).

In general, comparison of amino acid sequences is accomplished by aligning an amino acid sequence of a polypeptide of a known structure with the amino acid sequence of a the polypeptide of unknown structure. Amino acids in the sequences are then compared and groups of amino acids that are homologous are grouped together. This method detects conserved regions of the polypeptides and accounts for amino acid insertions and deletions. Homology between amino acid sequences can be determined by using commercially available algorithms (see also the description of homology above). In addition to those otherwise mentioned herein, mention is made too of the programs BLAST, gapped BLAST, BLASTN, BLASTP, and PSI-BLAST, provided by the National Center for Biotechnology Information. These programs are widely used in the art for this purpose and can align homologous regions of two amino acid sequences.

Once the amino acid sequence of the polypeptides with known and unknown structures are aligned, the structures of the conserved amino acids in a computer representation of the polypeptide with known structure are transferred to the corresponding amino acids of the polypeptide whose structure is unknown. For example, a tyrosine in the amino acid sequence of known structure may be replaced by a phenylalanine, the corresponding homologous amino acid in the amino acid sequence of unknown structure. The structures of amino acids located in non-conserved regions may be assigned manually using standard peptide geometries or by molecular simulation techniques, such as molecular dynamics. Refining the entire structure can be by molecular dynamics and/or energy minimization.

The aspects of the invention which employ the Fab 4E10 and/or KGND structure in silico may be equally applied to homologue models of Fab 4E10 and/or KGND obtained by the above aspect of the invention and this forms yet a further embodiment of the invention. Thus, having determined a conformation of a Fab 4E10 and/or KGND by the methods described herein, such a conformation may be used in a computer-based method of rational drug or compound design or identification as described herein.

The invention further provides a method for determining the structure of a binder of Fab 4E10 bound to Fab 4E10 comprising: providing a crystal of Fab 4E10, e.g., according to the invention, soaking the crystal with the binder, and determining the structure of the FAB 4E10-binder complex. Alternatively or additionally the FAB 4E10 and the binder may be cocrystallized.

The invention further provides systems, such as computer systems, intended to generate structures and/or perform rational drug or compound design for a Fab 4E10 or complex of Fab 4E10 and a potential binder. The system can contain: atomic co-ordinate data according to Table 1 and the Figures or derived therefrom by homology modeling, said data defining the three-dimensional structure of a Fab 4E10 or at least one sub-domain thereof; or structure factor data for Fab 4E10, said structure factor data being derivable from the atomic co-ordinate data of Table 1 and the Figures. The invention also involves computer readable media with: atomic coordinate data according to Table 1 and/or the Figures or derived therefrom by homology modeling, said data defining the three-dimensional structure of a Fab 4E10 or at least one sub-domain thereof; or structure factor data for Fab 4E10, said structure factor data being derivable from the atomic co-ordinate data of Table 1 and/or the Figures. “Computer readable media” refers to any media which can be read and accessed directly by a computer, and includes, but is not limited to: magnetic storage media such as floppy discs, hard storage medium and magnetic tape; optical storage media such as optical discs or CD-ROM; electrical storage media such as RAM and ROM; and hybrids of these categories, such as magnetic/optical media. By providing such computer readable media, the atomic co-ordinate data can be routinely accessed to model Fab 4E10 or a sub-domain thereof. For example RASMOL (Sayle et al., TIBS vol. 20 (1995), 374) is a publicly available software package which allows access and analysis of atomic coordinate data for structural determination and/or rational drug design. The invention further comprehends methods of doing business by providing access to such computer readable media and/or computer systems and/or atomic co-ordinate data to users; e.g., the media and/or atomic co-ordinate data can be accessible to a user, for instance on a subscription basis, via the Internet or a global communication/computer network; or, the computer system can be available to a user, on a subscription basis. Structure factor data, which are derivable from atomic co-ordinate data (see, e.g., Blundell et al., in Protein Crystallography, Academic Press, NY, London and San Francisco (1976)), are particularly useful for calculating electron density maps, e.g., difference Fourier electron density maps. Thus, there are additional uses for the computer readable media and/or computer systems and/or atomic co-ordinate data and additional reasons to provide them to users. A “computer system” refers to the hardware means, software means and data storage means used to analyze the atomic co-ordinate data of the present invention. The minimum hardware means of computer-based systems of the invention may comprise a central processing unit (CPU), input means, output means, and data storage means. Desirably, a monitor is provided to visualize structure data. The data storage means may be RAM or other means for accessing computer readable media of the invention. Examples of such systems are microcomputer workstations available from Silicon Graphics Incorporated and Sun Microsystems running Unix based, Linux, Windows NT or IBM OS/2 operating systems.

Accordingly, the invention further comprehends methods of transmitting information obtained in any method or step thereof described herein or any information described herein, e.g., via telecommunications, telephone, mass communications, mass media, presentations, interne, email, etc.

The invention also provides a method of analyzing a complex of Fab 4E10 and a potential binder comprising: employing X-ray crystallographic diffraction data from the complex and a three-dimensional structure of Fab 4E10 or at least a sub-domain thereof, to generate a different Fourier electron density map of the complex; advantageously, the three-dimensional structure being as defined by the atomic co-ordinate data according to Table 1 and/or the Figures.

Such complexes can be crystallized and analyzed using X-ray diffraction methods, e.g., according to the approaches described by Greer et al., 1994, and difference Fourier electron density maps can be calculated based on X-ray diffraction patterns of soaked or co-crystallized Fab 4E10 and the solved structure of uncomplexed Fab 4E10. These maps can then be used to determine whether and where a particular potential binder binds to Fab 4E10 and/or changes the conformation of Fab 4E10. Electron density maps can be calculated using programs such as those from the CCP4 computer package (Collaborative Computing Project, No. 4. The CCP4 Suite: Programs for Protein Crystallography, Acta Crystallographica, D50, 1994, 760-763). For map visualization and model building programs such as “QUANTA” (1994, San Diego, Calif.: Molecular Simulations, Jones et al., 1991) can be used.

Table 1 gives atomic co-ordinate data for Fab 4E10 complexed with KGND, and lists each atom by a unique number; the chemical element and its position for each amino acid residue (as determined by electron density maps and antibody sequence comparisons), the amino acid residue in which the element is located, the chain identifier, the number of the residue, coordinates (e.g., X, Y, Z) which define with respect to the crystallographic axes the atomic position (in Å) of the respective atom, the occupancy of the atom in the respective position, “B”, isotropic displacement parameter (in Å²) which accounts for movement of the atom around its atomic center, and atomic number. See also the text herein and the Figures.

Determination of the 3D structure of Fab 4E10 provides important information about the likely active/binding site(s) of Fab 4E10. This information may be used for rational design of Fab 4E10 binders, e.g., by computational techniques that identify possible binding ligands for the active site(s), by enabling linked-fragment approaches to drug design, and by enabling the identification and location of bound ligands using analyses such as X-ray crystallographic analysis.

Greer et al., supra, relates to an iterative approach to ligand design based on repeated sequences of computer modeling, protein-ligand complex formation, and X-ray analysis. Thymidylate synthase inhibitors were designed by Greer; and, Fab 4E10 binders may also be designed in this way. Using, for example, GRID (P. Goodford, 1985) or the solved 3D structure of Fab 4E10, a potential binder of Fab 4E10 may be designed that complements the functionalities of the FAB 4E10 active site(s). The potential binder can be synthesized, formed into a complex with Fab 4E10, and the complex then analyzed, e.g., by X-ray crystallography, NMR or a combination thereof, to identify the actual position of the bound compound.

Determination of the position of the potential binder compound in the complex allows determination of the interactions of it with Fab 4E10. This allows the skilled artisan to analyze the affinity and specificity of the compound for Fab 4E10, and to propose modifications to the compound to increase or decrease either or both of these properties. Thus, the structure and/or functional groups of the compound can then be adjusted, if necessary or desired, in view of the results from the analysis (e.g., X-ray analysis), and the synthesis and analysis sequence repeated until an optimized compound is obtained. Related approaches to structure-based drug and compound design are also discussed in other documents cited herein, as well as in Bohacek et al., 1996.

As a result of the determination of the Fab 4E10 3D structure, more purely computational techniques for rational drug and compound design may also be used to design Fab 4E10 binders and hence compounds that elicit anti-HIV antibodies; for example, automated ligand-receptor docking programs (see Jones et al., 1995) which require accurate information on the atomic coordinates of target receptors, may be used to design or identify potential Fab 4E10 binders.

Linked-fragment approaches to drug or compound design also require accurate information on the atomic co-ordinates of a target. Small compounds that have the potential to bind to regions of Fab 4E10 which in themselves may not be binder compounds may be assembled by chemical linkage to provide potential binders. Thus, the basic idea behind these approaches is to determine the binding locations of more than one, e.g., plural or a plurality of, ligands to a target molecule, and then construct a molecular scaffold to connect the ligands together in such a way that their relative binding positions are preserved. The ligands may be provided computationally and modeled in a computer system, or provided in an experimental setting, wherein crystals according to the invention are provided and more than one, e.g., plural or a plurality of, ligands soaked separately or in mixed pools into the crystal prior to analysis, e.g., X-ray analysis, and determination of their location.

The binding site of two or more ligands are determined and may be connected to thus form a potential lead compound that can be further refined, e.g., the iterative technique of Greer et al. For a virtual linked-fragment approach, see Verlinde et al., 1992; and for NMR and X-ray approaches, see Skuker et al., 1996; and Stout et al., 1998. The use of these or other approaches to design and/or identify Fab 4E10 binders and hence compounds that elicit anti-HIV antibodies (see, e.g., patent documents cited herein such as in the Background Section and documents cited therein, supra) is made possible by the determination of the Fab 4E10 structure.

Many of the techniques and approaches to structure-based described herein employ X-ray analysis to identify the binding position of a potential modulator in a complex with a protein. A common way of doing this is to perform X-ray crystallography on the complex, produce a difference Fourier electron density map, and associate a particular pattern of electron density with the potential modulator. However, to produce a map (See Blundell et al., supra), it is important to know the 3D structure of the protein beforehand (or at least the protein structure factors). Therefore, determination of the Fab 4E10 structure also allows difference Fourier electron density maps of complexes of Fab 4E10 with a potential modulator to be produced, which can greatly assist in the process of rational compound and/or drug design or identification.

The approaches to structure-based drug or compound design or identification described herein involve initial identification of possible compounds for interaction with the target molecule (in this case Fab 4E10), and thus elicit anti-HIV antibodies. Sometimes these compounds are known, e.g., from research literature. However, when they are not, or when novel compounds are wanted, a first stage of the drug or compound design or identification program may involve computer-based in silico screening of compound databases (such as the Cambridge Structural Database) with the aim of identifying compounds which interact with the active site or sites of the target bio-molecule (in this case Fab 4E10). Screening selection criteria may be based on pharmacokinetic properties such as metabolic stability and toxicity. However, determination of the Fab 4E10 structure allows the architecture and chemical nature of each Fab 4E10 active site to be identified, which in turn allows the geometric and functional constraints of a descriptor for the potential binder to be derived. The descriptor can be, therefore, a type of virtual 3D pharmacophore, which can also be used as selection criteria or filter for database screening.

Compounds which have a chemical structure selected using the invention, wherein said compounds are Fab 4E10 binders, form a further aspect of the invention; and, such compounds may be used in methods of medical treatments, such as for diagnosis, preventing or treating HIV or for eliciting antibodies for diagnosis of HIV, including use in vaccines. Further, such compounds may be used in the preparation of medicaments for such treatments or prevention, or compositions for diagnostic purposes. The compounds may be employed alone or in combination with other treatments, vaccines or preventatives; and, the compounds may be used in the preparation of combination medicaments for such treatments or prevention, or in kits containing the compound and the other treatment or preventative.

It is noted that these therapeutics can be a chemical compound, a composition comprising a polypeptide of the present invention and/or antibody elicited by such a chemical compound and/or portion thereof or a pharmaceutically acceptable salt or a composition comprising a polypeptide of the invention, and can be administered alone or as an active ingredient in combination with pharmaceutically acceptable carriers, diluents, and vehicles, as well as other active ingredients.

The compounds or compositions can be administered orally, subcutaneously or parenterally including intravenous, intraarterial, intramuscular, intraperitoneally, and intranasal administration as well as intrathecal and infusion techniques.

It is noted that humans are treated generally longer than the mice or other experimental animals which treatment has a length proportional to the length of the disease process and drug effectiveness. The doses may be single doses or multiple doses over a period of several days, but single doses are preferred. Thus, one can scale up from animal experiments, e.g., rats, mice, and the like, to humans, by techniques from this disclosure and documents cited herein and the knowledge in the art, without undue experimentation.

The treatment generally has a length proportional to the length of the disease process and drug effectiveness and the patient being treated.

When administering a therapeutic of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various amounts of the other ingredients, as desired.

A pharmacological formulation of the present invention, e.g., comprising a therapeutic compound or polypeptide of the present invention, can be administered to the patient in an injectable formulation containing any compatible carrier, such as various vehicles, adjuvants, additives, and diluents; or the compounds utilized in the present invention can be administered parenterally to the patient in the form of slow-release subcutaneous implants or targeted delivery systems such as monoclonal antibodies, iontophoretic, polymer matrices, liposomes, and microspheres.

A pharmacological formulation of the compound and composition comprising a polypeptide utilized in the present invention can be administered orally to the patient. Conventional methods such as administering the compounds in tablets, suspensions, solutions, emulsions, capsules, powders, syrups and the like are usable. Known techniques which deliver the compound orally or intravenously and retain the biological activity are preferred.

In one embodiment, a formulation of the present invention can be administered initially, and thereafter maintained by further administration. For instance, a formulation of the invention can be administered in one type of composition and thereafter further administered in a different or the same type of composition. For example, a formulation of the invention can be administered by intravenous injection to bring blood levels to a suitable level. The patient's levels are then maintained by an oral dosage form, although other forms of administration, dependent upon the patient's condition, can be used. In the instance of a vaccine composition, the vaccine may be administered as a single dose, or the vaccine may incorporate set booster doses. For example, booster doses may comprises variants in order to provide protection against multiple clades of HIV.

The quantity to be administered will vary for the patient being treated and whether the administration is for treatment or prevention and will vary from a few micrograms to a few milligrams for an average 70 kg patient, e.g., 5 micrograms to 5 milligrams such as 500 micrograms, or about 100 ng/kg of body weight to 100 mg/kg of body weight per administration and preferably will be from 10 pg/kg to 10 mg/kg per administration. Typically, however, the antigen is present in an amount on, the order of micrograms to milligrams, or, about 0.001 to about 20 wt %, preferably about 0.01 to about 10 wt %, and most preferably about 0.05 to about 5 wt %.

Of course, for any composition to be administered to an animal or human, including the components thereof, and for any particular method of administration, it is preferred to determine therefor: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable immunological response, such as by titrations of sera and analysis thereof for antibodies or antigens, e.g., by ELISA and/or RFFIT analysis. Such determinations do not require undue experimentation from the knowledge of the skilled artisan, this disclosure and the documents cited herein. And, the time for sequential administrations can be ascertained without undue experimentation. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention. Typically, an adjuvant or additive is commonly used as 0.001 to 50 wt % solution in phosphate buffered saline, and the active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %. Such determinations do not require undue experimentation from the knowledge of the skilled artisan, this disclosure and the documents cited herein. And, the time for sequential administrations can be ascertained without undue experimentation.

Examples of compositions comprising a therapeutic of the invention include liquid preparations for orifice, e.g., oral, nasal, anal, vaginal, peroral, intragastric, mucosal (e.g., perlingual, alveolar, gingival, olfactory or respiratory mucosa) etc., administration such as suspensions, syrups or elixirs; and, preparations for parenteral, subcutaneous, intradermal, intramuscular or intravenous administration (e.g., injectable administration), such as sterile suspensions or emulsions. Such compositions may be in admixture with a suitable carrier, diluent, or excipient such as sterile water, physiological saline, glucose or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON′S PHARMACEUTICAL SCIENCE”, 17th edition, 1985, incorporated herein by reference, may be consulted to prepare suitable preparations, without undue experimentation.

Compositions of the invention, are conveniently provided as liquid preparations, e.g., isotonic aqueous solutions, suspensions, emulsions or viscous compositions which may be buffered to a selected pH. If digestive tract absorption is preferred, compositions of the invention can be in the “solid” form of pills, tablets, capsules, caplets and the like, including “solid” preparations which are time-released or which have a liquid filling, e.g., gelatin covered liquid, whereby the gelatin is dissolved in the stomach for delivery to the gut. If nasal or respiratory (mucosal) administration is desired, compositions may be in a form and dispensed by a squeeze spray dispenser, pump dispenser or aerosol dispenser. Aerosols are usually under pressure by means of a hydrocarbon. Pump dispensers can preferably dispense a metered dose or, a dose having a particular particle size.

Compositions of the invention can contain pharmaceutically acceptable flavors and/or colors for rendering them more appealing, especially if they are administered orally. The viscous compositions may be in the form of gels, lotions, ointments, creams and the like (e.g., for transdermal administration) and will typically contain a sufficient amount of a thickening agent so that the viscosity is from about 2500 to 6500 cps, although more viscous compositions, even up to 10,000 cps may be employed. Viscous compositions have a viscosity preferably of 2500 to 5000 cps, since above that range they become more difficult to administer. However, above that range, the compositions can approach solid or gelatin forms which are then easily administered as a swallowed pill for oral ingestion.

Liquid preparations are normally easier to prepare than gels, other viscous compositions, and solid compositions. Additionally, liquid compositions are somewhat more convenient to administer, especially by injection or orally. Viscous compositions, on the other hand, can be formulated within the appropriate viscosity range to provide longer contact periods with mucosa, such as the lining of the stomach or nasal mucosa.

Obviously, the choice of suitable carriers and other additives will depend on the exact route of administration and the nature of the particular dosage form, e.g., liquid dosage form (e.g., whether the composition is to be formulated into a solution, a suspension, gel or another liquid form), or solid dosage form (e.g., whether the composition is to be formulated into a pill, tablet, capsule, caplet, time release form or liquid-filled form).

Solutions, suspensions and gels, normally contain a major amount of water (preferably purified water) in addition to the active compound. Minor amounts of other ingredients such as pH adjusters (e.g., a base such as NaOH), emulsifiers or dispersing agents, buffering agents, preservatives, wetting agents, jelling agents, (e.g., methylcellulose), colors and/or flavors may also be present. The compositions can be isotonic, i.e., it can have the same osmotic pressure as blood and lacrimal fluid.

The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions may be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

A pharmaceutically acceptable preservative can be employed to increase the shelf-life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight although there may be appreciable variation depending upon the agent selected.

Those skilled in the art will recognize that the components of the compositions should be selected to be chemically inert with respect to the active compound. This will present no problem to those skilled in chemical and pharmaceutical principles, or problems can be readily avoided by reference to standard texts or by simple experiments (not involving undue experimentation), from this disclosure and the documents cited herein.

It is generally envisaged that compounds and compositions of the invention will be administered by injection, as such compounds are to elicit anti-HIV antibodies, and the skilled artisan can, from this disclosure and the knowledge in the art, formulate compounds and compositions identified by herein methods for administration by injection and administer such compounds and compositions by injection.

The inventive compositions of this invention are prepared by mixing the ingredients following generally accepted procedures. For example the selected components may be simply mixed in a blender, or other standard device to produce a concentrated mixture which may then be adjusted to the final concentration and viscosity by the addition of water or thickening agent and possibly a buffer to control pH or an additional solute to control tonicity. Generally the pH may be from about 3 to 7.5. Compositions can be administered in dosages and by techniques well known to those skilled in the medical arts taking into consideration such factors as the age, sex, weight, and condition of the particular patient, and the composition form used for administration (e.g., solid vs. liquid). Dosages for humans or other mammals can be determined without undue experimentation by the skilled artisan, from this disclosure, the documents cited herein, and the knowledge in the art.

Suitable regimes for initial administration and further doses or for sequential administrations also are variable, may include an initial administration followed by subsequent administrations; but nonetheless, may be ascertained by the skilled artisan, from this disclosure, the documents cited herein, and the knowledge in the art.

Accordingly, the invention comprehends; in further aspects, methods for preparing therapeutic or preventive compositions including an active agent, ingredient or compound or Fab 4E10 binder as from inventive methods herein for ascertaining compounds that bind to, as well as to methods for inhibiting HIV or eliciting antibodies against HIV by administering a compound or compounds that bind to Fab 4E10.

Furthermore, as discussed herein, compounds which bind to Fab 4E10 are useful in generating antibodies, which are themselves useful in assays as well as in therapeutics as well as diagnostics; and, the compounds which bind to Fab 4E10 are useful for detecting anti-HIV antibodies in a sample. From documents cited herein, one can readily make and use such antibodies, and methods for producing monoclonal antibodies are well known to those of ordinary skill in the art, see, e.g., U.S. Pat. Nos. 4,196,265 and 6,221,645. Thus, the compounds that bind to Fab 4E10 can be used to generate antibodies and the antibodies can be used, without undue experimentation, e.g., to detect HIV immunogens, antigens or epitopes in a sample.

The invention is further described by the following non-limiting example(s), given by way of illustration.

### EXAMPLES

### Example 1

### Crystallization of Fab 4E10 Complex

Fab 4E10 was obtained from Polymun, Herman Katinger, and is otherwise available as described in documents cited/incorporated by reference herein. Briefly, Fab 4E10 was obtained by antibody producing hybridomas that were generated by a combined polyethylene glycol/electrofusion method. PBMC from 10 asymptomatic HIV-1 positive donors were fused with the mouse-human heteromyeloma cell line CB-F7. Hybridoma supernatants were screened for HIV-specific antibody production and positive clones were further analyzed by ELISA, Western blot, and immunofluorescence assays. In order to enable safe mass production and to change the isotype of 2175-and 4E10 from IgG3 to IgG1 the antibodies were expressed recombinantly in Chinese Hamster Ovary cells (CHO) as IgG1.

The term “4E10-IgG3” exclusively refers to the known IgG3 variant and the term “4E10-IgG1” to the IgG1 variant of 4E10. Mab 4E10IgG3 is produced by a hybridoma cell line deposited at ECACC under Accession Nr. 90091703, while 4E10-IgG1 is expressed by a CHO cell line (deposited under the Budapest Treaty at ECACC Acc.Nr. 01 1 10665). Both variants recognize the same epitope on gp41 of HIV.

The minimum binding epitope (core epitope) of 4E10 is entirely present on peptide 2031 and is located subsequent to the ELDKWAS (SEQ ID NO: 82) epitope of 2F5 and within the aa sequence LWNWFDITNWL (SEQ ID NO: 83) (a.a. positions 670-680 of gp41; numbering according to TCLA isolate HTLV-III MN). More detailed mapping using smaller peptides revealed a core epitope of 5 amino acids comprising the aa sequence WFXIT (SEQ ID NO: 78) (a.a. 673-677 of gp41 of HTLV-III MN). The X may preferably be D, N, S, or T, although other amino acids are possible.

Fab 4E10 was contacted with KGND, which was synthesized using standard protein synthesis techniques. Crystals were grown by the vapor diffusion method under the following conditions: 10% PEG (polyethylene glycol), 0.1 M sodium citrate pH 5, and 10 mM hexaminecobalt trichloride. The formed crystals are as described herein and in the Figures, with atomic coordinates as set forth in Table 1, determined by X-ray diffraction using a Synchrotron Radiation source and otherwise standard XRD methods (see, e.g., documents cited/incorporated by reference herein). The Figures identify relevant regions of KGND and Fab 4E10, and provide comparisons thereof, all of which may be employed by the skilled artisan in the practice of embodiments of the invention.

TABLE 1
Atomic Coordinates (see also FIGS.):

| | | | | | | | | | | |
|---|---|---|---|---|---|---|---|---|---|---|
| HELIX | 6 | 6 | PRO | H | 84 | ASP | H | 86 | 5 | 3 |
| HELIX | 7 | 7 | ASN | H | 162 | GLY | H | 164 | 5 | 3 |
| HELIX | 8 | 8 | LYS | H | 213 | SER | H | 215 | 5 | 3 |
| HELIX | 1 | 1 | PRO | L | 80 | ASP | L | 82 | 5 | 3 |
| HELIX | 2 | 2 | SER | L | 121 | GLY | L | 128 | 1 | 8 |
| HELIX | 3 | 3 | LYS | L | 183 | GLU | L | 187 | 1 | 5 |
| HELIX | 4 | 4 | ASP | P | 7 | LYS | P | 13 | 5 | 12 |

| | | | | | | | | | | | | | | | | | | |
|---|---|---|---|---|---|---|---|---|---|---|---|---|---|---|---|---|---|---|
| SHEET | 1 | E | 4 | GLN | H | 3 | SER | H | 7 | 0 | | | | | | | | |
| SHEET | 2 | E | 4 | VAL | H | 18 | SER | H | 25 | −1 | N | SER | H | 25 | O | GLN | H | 3 |
| SHEET | 3 | E | 4 | THR | H | 77 | LEU | H | 82 | −1 | N | LEU | H | 82 | O | VAL | H | 18 |
| SHEET | 4 | E | 4 | ILE | H | 67 | ASP | H | 72 | −1 | N | ASP | H | 72 | O | THR | H | 77 |
| SHEET | 1 | F | 5 | THR | H | 107 | VAL | H | 109 | 0 | | | | | | | | |
| SHEET | 2 | F | 5 | ALA | H | 88 | GLU | H | 95 | −1 | N | TYR | H | 90 | O | THR | H | 107 |
| SHEET | 3 | F | 5 | ILE | H | 34 | GLN | H | 39 | −1 | N | GLN | H | 39 | O | VAL | H | 89 |
| SHEET | 4 | F | 5 | LEU | H | 45 | ILE | H | 52 | −1 | N | ILE | H | 51 | O | SER | H | 35 |
| SHEET | 1 | H | 4 | SER | H | 120 | LEU | H | 124 | 0 | | | | | | | | |
| SHEET | 2 | H | 4 | THR | H | 137 | TYR | H | 147 | −1 | N | LYS | H | 145 | O | SER | H | 120 |
| SHEET | 3 | H | 4 | TYR | H | 185 | PRO | H | 194 | −1 | N | VAL | H | 193 | O | ALA | H | 138 |
| SHEET | 4 | H | 4 | VAL | H | 171 | THR | H | 173 | −1 | N | HIS | H | 172 | O | VAL | H | 190 |
| SHEET | 1 | I | 3 | THR | H | 153 | TRP | H | 157 | 0 | | | | | | | | |
| SHEET | 2 | I | 3 | THR | H | 205 | HIS | H | 212 | −1 | N | ASN | H | 211 | O | THR | H | 153 |
| SHEET | 3 | I | 3 | THR | H | 217 | LYS | H | 222 | −1 | N | LYS | H | 222 | O | THR | H | 205 |
| SHEET | 1 | A | 4 | THR | L | 10 | SER | L | 12 | 0 | | | | | | | | |
| SHEET | 2 | A | 4 | THR | L | 102 | GLU | L | 105 | 1 | N | LYS | L | 103 | O | MET | L | 11 |
| SHEET | 3 | A | 4 | ALA | L | 84 | GLN | L | 90 | −1 | N | TYR | L | 86 | O | THR | L | 102 |
| SHEET | 4 | A | 4 | LEU | L | 33 | GLN | L | 38 | −1 | N | GLN | L | 38 | O | THR | L | 85 |
| SHEET | 1 | B | 3 | VAL | L | 19 | ARG | L | 24 | 0 | | | | | | | | |
| SHEET | 2 | B | 3 | ASP | L | 70 | ILE | L | 75 | −1 | N | ILE | L | 75 | O | VAL | L | 19 |
| SHEET | 3 | B | 3 | PHE | L | 62 | SER | L | 67 | −1 | N | SER | L | 67 | O | ASP | L | 70 |
| SHEET | 1 | C | 4 | SER | L | 114 | PHE | L | 118 | 0 | | | | | | | | |
| SHEET | 2 | C | 4 | THR | L | 129 | ASN | L | 137 | −1 | N | ASN | L | 137 | O | SER | L | 114 |
| SHEET | 3 | C | 4 | LEU | L | 175 | SER | L | 182 | −1 | N | LEU | L | 181 | O | ALA | L | 130 |
| SHEET | 4 | C | 4 | SER | L | 159 | VAL | L | 163 | −1 | N | SER | L | 162 | O | SER | L | 176 |
| SHEET | 1 | D | 3 | ALA | L | 144 | VAL | L | 150 | 0 | | | | | | | | |
| SHEET | 2 | D | 3 | VAL | L | 191 | HIS | L | 198 | −1 | N | THR | L | 197 | O | LYS | L | 145 |
| SHEET | 3 | D | 3 | VAL | L | 205 | ASN | L | 210 | −1 | N | PHE | L | 209 | O | TYR | L | 192 |

| | | | | | | | |
|---|---|---|---|---|---|---|---|
| SSBOND | 3 | CYS | H | 22 | CYS | H | 92 |
| SSBOND | 4 | CYS | H | 142 | CYS | H | 208 |
| SSBOND | 1 | CYS | L | 23 | CYS | L | 88 |
| SSBOND | 2 | CYS | L | 134 | CYS | L | 194 |

| | | | | | | | | | | | |
|---|---|---|---|---|---|---|---|---|---|---|---|
| ATOM | 1634 | CB | GLN | H | 1 | 35.464 | 4.610 | −22.540 | 1.00 | 44.42 | H |
| ATOM | 1635 | CG | GLN | H | 1 | 36.944 | 4.690 | −22.899 | 1.00 | 47.07 | H |
| ATOM | 1636 | CD | GLN | H | 1 | 37.203 | 5.514 | −24.158 | 1.00 | 49.57 | H |
| ATOM | 1637 | OE1 | GLN | H | 1 | 36.763 | 6.660 | −24.267 | 1.00 | 53.18 | H |
| ATOM | 1638 | NE2 | GLN | H | 1 | 37.919 | 4.930 | −25.111 | 1.00 | 49.25 | H |
| ATOM | 1639 | C | GLN | H | 1 | 33.172 | 4.442 | −23.535 | 1.00 | 39.46 | H |
| ATOM | 1640 | O | GLN | H | 1 | 32.905 | 5.528 | −24.050 | 1.00 | 38.74 | H |
| ATOM | 1641 | N | GLN | H | 1 | 35.141 | 4.133 | −24.947 | 1.00 | 42.08 | H |
| ATOM | 1642 | CA | GLN | H | 1 | 34.599 | 3.894 | −23.580 | 1.00 | 41.17 | H |
| ATOM | 1643 | N | VAL | H | 2 | 32.263 | 3.687 | −22.925 | 1.00 | 35.96 | H |
| ATOM | 1644 | CA | VAL | H | 2 | 30.865 | 4.090 | −22.830 | 1.00 | 33.37 | H |
| ATOM | 1645 | CB | VAL | H | 2 | 29.913 | 2.929 | −23.213 | 1.00 | 31.89 | H |
| ATOM | 1646 | CG1 | VAL | H | 2 | 28.459 | 3.346 | −22.977 | 1.00 | 27.56 | H |
| ATOM | 1647 | CG2 | VAL | H | 2 | 30.136 | 2.528 | −24.667 | 1.00 | 28.18 | H |
| ATOM | 1648 | C | VAL | H | 2 | 30.458 | 4.572 | −21.449 | 1.00 | 33.18 | H |
| ATOM | 1649 | O | VAL | H | 2 | 30.570 | 3.842 | −20.465 | 1.00 | 33.53 | H |
| ATOM | 1650 | N | GLN | H | 3 | 29.985 | 5.809 | −21.380 | 1.00 | 33.14 | H |
| ATOM | 1651 | CA | GLN | H | 3 | 29.527 | 6.368 | −20.119 | 1.00 | 33.32 | H |
| ATOM | 1652 | CB | GLN | H | 3 | 30.416 | 7.532 | −19.669 | 1.00 | 36.32 | H |
| ATOM | 1653 | CG | GLN | H | 3 | 29.883 | 8.221 | −18.415 | 1.00 | 42.78 | H |
| ATOM | 1654 | CD | GLN | H | 3 | 30.801 | 9.303 | −17.873 | 1.00 | 45.73 | H |
| ATOM | 1655 | OE1 | GLN | H | 3 | 31.125 | 10.267 | −18.566 | 1.00 | 46.89 | H |
| ATOM | 1656 | NE2 | GLN | H | 3 | 31.213 | 9.151 | −16.617 | 1.00 | 47.02 | H |
| ATOM | 1657 | C | GLN | H | 3 | 28.094 | 6.858 | −20.292 | 1.00 | 31.38 | H |
| ATOM | 1658 | O | GLN | H | 3 | 27.782 | 7.566 | −21.253 | 1.00 | 30.46 | H |
| ATOM | 1659 | N | LEU | H | 4 | 27.224 | 6.462 | −19.370 | 1.00 | 26.86 | H |
| ATOM | 1660 | CA | LEU | H | 4 | 25.827 | 6.877 | −19.402 | 1.00 | 25.40 | H |
| ATOM | 1661 | CB | LEU | H | 4 | 24.895 | 5.660 | −19.296 | 1.00 | 21.39 | H |
| ATOM | 1662 | CG | LEU | H | 4 | 25.059 | 4.587 | −20.376 | 1.00 | 23.97 | H |
| ATOM | 1663 | CD1 | LEU | H | 4 | 24.063 | 3.462 | −20.136 | 1.00 | 23.04 | H |
| ATOM | 1664 | CD2 | LEU | H | 4 | 24.846 | 5.195 | −21.747 | 1.00 | 24.82 | H |
| ATOM | 1665 | C | LEU | H | 4 | 25.629 | 7.797 | −18.204 | 1.00 | 23.85 | H |
| ATOM | 1666 | O | LEU | H | 4 | 25.958 | 7.428 | −17.080 | 1.00 | 25.02 | H |
| ATOM | 1667 | N | VAL | H | 5 | 25.103 | 8.993 | −18.443 | 1.00 | 21.98 | H |
| ATOM | 1668 | CA | VAL | H | 5 | 24.892 | 9.952 | −17.365 | 1.00 | 21.74 | H |
| ATOM | 1669 | CB | VAL | H | 5 | 25.715 | 11.240 | −17.600 | 1.00 | 22.48 | H |
| ATOM | 1670 | CG1 | VAL | H | 5 | 25.562 | 12.177 | −16.413 | 1.00 | 20.70 | H |
| ATOM | 1671 | CG2 | VAL | H | 5 | 27.186 | 10.882 | −17.830 | 1.00 | 21.74 | H |
| ATOM | 1672 | C | VAL | H | 5 | 23.421 | 10.311 | −17.284 | 1.00 | 21.36 | H |
| ATOM | 1673 | O | VAL | H | 5 | 22.830 | 10.786 | −18.256 | 1.00 | 21.16 | H |
| ATOM | 1674 | N | GLU | H | 6 | 22.836 | 10.094 | −16.114 | 1.00 | 20.79 | H |
| ATOM | 1675 | CA | GLU | H | 6 | 21.420 | 10.361 | −15.906 | 1.00 | 20.25 | H |
| ATOM | 1676 | CB | GLU | H | 6 | 20.800 | 9.206 | −15.109 | 1.00 | 17.82 | H |
| ATOM | 1677 | CG | GLU | H | 6 | 20.923 | 7.856 | −15.811 | 1.00 | 16.88 | H |
| ATOM | 1678 | CD | GLU | H | 6 | 20.220 | 6.717 | −15.074 | 1.00 | 18.65 | H |
| ATOM | 1679 | OE1 | GLU | H | 6 | 19.098 | 6.933 | −14.557 | 1.00 | 15.64 | H |
| ATOM | 1680 | OE2 | GLU | H | 6 | 20.787 | 5.599 | −15.032 | 1.00 | 15.93 | H |
| ATOM | 1681 | C | GLU | H | 6 | 21.143 | 11.679 | −15.197 | 1.00 | 19.77 | H |
| ATOM | 1682 | O | GLU | H | 6 | 22.021 | 12.251 | −14.550 | 1.00 | 20.51 | H |
| ATOM | 1683 | N | SER | H | 7 | 19.913 | 12.161 | −15.331 | 1.00 | 20.87 | H |
| ATOM | 1684 | CA | SER | H | 7 | 19.509 | 13.394 | −14.672 | 1.00 | 20.94 | H |
| ATOM | 1685 | CB | SER | H | 7 | 18.169 | 13.878 | −15.239 | 1.00 | 20.14 | H |
| ATOM | 1686 | OG | SER | H | 7 | 17.293 | 12.788 | −15.494 | 1.00 | 23.20 | H |
| ATOM | 1687 | C | SER | H | 7 | 19.427 | 13.146 | −13.157 | 1.00 | 19.57 | H |
| ATOM | 1688 | O | SER | H | 7 | 19.364 | 11.996 | −12.708 | 1.00 | 19.67 | H |
| ATOM | 1689 | N | GLY | H | 8 | 19.437 | 14.224 | −12.378 | 1.00 | 19.48 | H |
| ATOM | 1690 | CA | GLY | H | 8 | 19.408 | 14.108 | −10.928 | 1.00 | 16.62 | H |
| ATOM | 1691 | C | GLY | H | 8 | 18.120 | 13.642 | −10.275 | 1.00 | 16.61 | H |
| ATOM | 1692 | O | GLY | H | 8 | 17.067 | 13.614 | −10.903 | 1.00 | 14.54 | H |
| ATOM | 1693 | N | ALA | H | 9 | 18.223 | 13.280 | −8.998 | 1.00 | 15.56 | H |
| ATOM | 1694 | CA | ALA | H | 9 | 17.090 | 12.806 | −8.208 | 1.00 | 17.26 | H |
| ATOM | 1695 | CB | ALA | H | 9 | 17.507 | 12.642 | −6.737 | 1.00 | 12.74 | H |
| ATOM | 1696 | C | ALA | H | 9 | 15.940 | 13.797 | −8.308 | 1.00 | 19.39 | H |
| ATOM | 1697 | O | ALA | H | 9 | 16.160 | 14.991 | −8.481 | 1.00 | 19.15 | H |
| ATOM | 1698 | N | GLU | H | 10 | 14.712 | 13.310 | −8.194 | 1.00 | 19.88 | H |
| ATOM | 1699 | CA | GLU | H | 10 | 13.575 | 14.208 | −8.279 | 1.00 | 21.25 | H |
| ATOM | 1700 | CB | GLU | H | 10 | 13.198 | 14.431 | −9.741 | 1.00 | 25.39 | H |
| ATOM | 1701 | CG | GLU | H | 10 | 12.243 | 15.597 | −9.932 | 1.00 | 30.96 | H |
| ATOM | 1702 | CD | GLU | H | 10 | 11.905 | 15.852 | −11.385 | 1.00 | 30.30 | H |
| ATOM | 1703 | OE1 | GLU | H | 10 | 11.200 | 16.842 | −11.653 | 1.00 | 33.08 | H |
| ATOM | 1704 | OE2 | GLU | H | 10 | 12.337 | 15.065 | −12.254 | 1.00 | 31.11 | H |
| ATOM | 1705 | C | GLU | H | 10 | 12.350 | 13.750 | −7.505 | 1.00 | 19.69 | H |
| ATOM | 1706 | O | GLU | H | 10 | 12.026 | 12.563 | −7.476 | 1.00 | 19.95 | H |
| ATOM | 1707 | N | VAL | H | 11 | 11.681 | 14.708 | −6.870 | 1.00 | 17.87 | H |
| ATOM | 1708 | CA | VAL | H | 11 | 10.470 | 14.444 | −6.104 | 1.00 | 16.18 | H |
| ATOM | 1709 | CB | VAL | H | 11 | 10.361 | 15.388 | −4.883 | 1.00 | 17.92 | H |
| ATOM | 1710 | CG1 | VAL | H | 11 | 8.998 | 15.221 | −4.200 | 1.00 | 14.80 | H |
| ATOM | 1711 | CG2 | VAL | H | 11 | 11.488 | 15.092 | −3.902 | 1.00 | 16.82 | H |
| ATOM | 1712 | C | VAL | H | 11 | 9.289 | 14.693 | −7.036 | 1.00 | 15.88 | H |
| ATOM | 1713 | O | VAL | H | 11 | 9.238 | 15.724 | −7.696 | 1.00 | 14.46 | H |
| ATOM | 1714 | N | LYS | H | 12 | 8.358 | 13.740 | −7.100 | 1.00 | 15.37 | H |
| ATOM | 1715 | CA | LYS | H | 12 | 7.175 | 13.855 | −7.952 | 1.00 | 14.41 | H |
| ATOM | 1716 | CB | LYS | H | 12 | 7.238 | 12.854 | −9.106 | 1.00 | 14.67 | H |
| ATOM | 1717 | CG | LYS | H | 12 | 8.473 | 12.964 | −9.964 | 1.00 | 19.76 | H |
| ATOM | 1718 | CD | LYS | H | 12 | 8.567 | 14.308 | −10.663 | 1.00 | 21.24 | H |
| ATOM | 1719 | CE | LYS | H | 12 | 7.433 | 14.496 | −11.646 | 1.00 | 23.39 | H |
| ATOM | 1720 | NZ | LYS | H | 12 | 7.744 | 15.609 | −12.583 | 1.00 | 24.49 | H |
| ATOM | 1721 | C | LYS | H | 12 | 5.899 | 13.588 | −7.156 | 1.00 | 13.60 | H |
| ATOM | 1722 | O | LYS | H | 12 | 5.826 | 12.642 | −6.375 | 1.00 | 13.46 | H |
| ATOM | 1723 | N | ARG | H | 13 | 4.892 | 14.422 | −7.377 | 1.00 | 12.93 | H |
| ATOM | 1724 | CA | ARG | H | 13 | 3.622 | 14.282 | −6.692 | 1.00 | 13.80 | H |
| ATOM | 1725 | CB | ARG | H | 13 | 2.784 | 15.560 | −6.854 | 1.00 | 18.82 | H |
| ATOM | 1726 | CG | ARG | H | 13 | 3.376 | 16.819 | −6.207 | 1.00 | 22.51 | H |
| ATOM | 1727 | CD | ARG | H | 13 | 3.368 | 16.734 | −4.687 | 1.00 | 28.33 | H |
| ATOM | 1728 | NE | ARG | H | 13 | 3.852 | 17.969 | −4.067 | 1.00 | 31.48 | H |
| ATOM | 1729 | CZ | ARG | H | 13 | 4.979 | 18.070 | −3.366 | 1.00 | 33.17 | H |
| ATOM | 1730 | NH1 | ARG | H | 13 | 5.756 | 17.004 | −3.181 | 1.00 | 28.06 | H |
| ATOM | 1731 | NH2 | ARG | H | 13 | 5.336 | 19.245 | −2.855 | 1.00 | 32.70 | H |
| ATOM | 1732 | C | ARG | H | 13 | 2.850 | 13.111 | −7.275 | 1.00 | 12.60 | H |
| ATOM | 1733 | O | ARG | H | 13 | 2.986 | 12.783 | −8.451 | 1.00 | 12.87 | H |
| ATOM | 1734 | N | PRO | H | 14 | 2.036 | 12.454 | −6.453 | 1.00 | 12.42 | H |
| ATOM | 1735 | CD | PRO | H | 14 | 1.872 | 12.626 | −4.999 | 1.00 | 11.72 | H |
| ATOM | 1736 | CA | PRO | H | 14 | 1.252 | 11.321 | −6.952 | 1.00 | 12.90 | H |
| ATOM | 1737 | CB | PRO | H | 14 | 0.418 | 10.929 | −5.737 | 1.00 | 13.82 | H |
| ATOM | 1738 | CG | PRO | H | 14 | 1.308 | 11.287 | −4.580 | 1.00 | 13.36 | H |
| ATOM | 1739 | C | PRO | H | 14 | 0.375 | 11.773 | −8.137 | 1.00 | 14.87 | H |
| ATOM | 1740 | O | PRO | H | 14 | −0.194 | 12.868 | −8.117 | 1.00 | 15.18 | H |
| ATOM | 1741 | N | GLY | H | 15 | 0.283 | 10.938 | −9.167 | 1.00 | 14.17 | H |
| ATOM | 1742 | CA | GLY | H | 15 | −0.537 | 11.273 | −10.316 | 1.00 | 12.35 | H |
| ATOM | 1743 | C | GLY | H | 15 | 0.204 | 12.000 | −11.421 | 1.00 | 14.60 | H |
| ATOM | 1744 | O | GLY | H | 15 | −0.261 | 12.043 | −12.564 | 1.00 | 13.70 | H |
| ATOM | 1745 | N | SER | H | 16 | 1.359 | 12.569 | −11.096 | 1.00 | 13.34 | H |
| ATOM | 1746 | CA | SER | H | 16 | 2.128 | 13.285 | −12.094 | 1.00 | 15.40 | H |
| ATOM | 1747 | CB | SER | H | 16 | 3.080 | 14.289 | −11.424 | 1.00 | 15.52 | H |
| ATOM | 1748 | OG | SER | H | 16 | 4.182 | 13.644 | −10.806 | 1.00 | 15.76 | H |
| ATOM | 1749 | C | SER | H | 16 | 2.920 | 12.319 | −12.973 | 1.00 | 15.45 | H |
| ATOM | 1750 | O | SER | H | 16 | 2.825 | 11.103 | −12.828 | 1.00 | 14.60 | H |
| ATOM | 1751 | N | SER | H | 17 | 3.675 | 12.883 | −13.906 | 1.00 | 15.38 | H |
| ATOM | 1752 | CA | SER | H | 17 | 4.509 | 12.108 | −14.806 | 1.00 | 17.26 | H |
| ATOM | 1753 | CB | SER | H | 17 | 4.127 | 12.366 | −16.274 | 1.00 | 20.21 | H |
| ATOM | 1754 | OG | SER | H | 17 | 2.817 | 11.902 | −16.558 | 1.00 | 26.02 | H |
| ATOM | 1755 | C | SER | H | 17 | 5.944 | 12.553 | −14.585 | 1.00 | 16.04 | H |
| ATOM | 1756 | O | SER | H | 17 | 6.207 | 13.732 | −14.339 | 1.00 | 16.03 | H |
| ATOM | 1757 | N | VAL | H | 18 | 6.873 | 11.611 | −14.665 | 1.00 | 14.04 | H |
| ATOM | 1758 | CA | VAL | H | 18 | 8.274 | 11.942 | −14.492 | 1.00 | 14.27 | H |
| ATOM | 1759 | CB | VAL | H | 18 | 8.911 | 11.121 | −13.343 | 1.00 | 13.52 | H |
| ATOM | 1760 | CG1 | VAL | H | 18 | 8.880 | 9.630 | −13.678 | 1.00 | 15.10 | H |
| ATOM | 1761 | CG2 | VAL | H | 18 | 10.342 | 11.577 | −13.108 | 1.00 | 10.10 | H |
| ATOM | 1762 | C | VAL | H | 18 | 9.035 | 11.651 | −15.775 | 1.00 | 15.21 | H |
| ATOM | 1763 | O | VAL | H | 18 | 8.682 | 10.741 | −16.521 | 1.00 | 16.43 | H |
| ATOM | 1764 | N | SER | H | 19 | 10.069 | 12.438 | −16.039 | 1.00 | 16.54 | H |
| ATOM | 1765 | CA | SER | H | 19 | 10.910 | 12.224 | −17.205 | 1.00 | 18.70 | H |
| ATOM | 1766 | CB | SER | H | 19 | 10.812 | 13.394 | −18.189 | 1.00 | 19.75 | H |
| ATOM | 1767 | OG | SER | H | 19 | 9.548 | 13.433 | −18.829 | 0.70 | 16.84 | H |
| ATOM | 1768 | C | SER | H | 19 | 12.343 | 12.101 | −16.707 | 1.00 | 19.36 | H |
| ATOM | 1769 | O | SER | H | 19 | 12.822 | 12.958 | −15.975 | 1.00 | 18.14 | H |
| ATOM | 1770 | N | VAL | H | 20 | 13.016 | 11.023 | −17.084 | 1.00 | 18.70 | H |
| ATOM | 1771 | CA | VAL | H | 20 | 14.396 | 10.822 | −16.673 | 1.00 | 17.31 | H |
| ATOM | 1772 | CB | VAL | H | 20 | 14.562 | 9.512 | −15.859 | 1.00 | 20.50 | H |
| ATOM | 1773 | CG1 | VAL | H | 20 | 16.014 | 9.356 | −15.393 | 1.00 | 16.20 | H |
| ATOM | 1774 | CG2 | VAL | H | 20 | 13.624 | 9.524 | −14.659 | 1.00 | 17.53 | H |
| ATOM | 1775 | C | VAL | H | 20 | 15.225 | 10.746 | −17.942 | 1.00 | 16.56 | H |
| ATOM | 1776 | O | VAL | H | 20 | 14.833 | 10.098 | −18.903 | 1.00 | 16.58 | H |
| ATOM | 1777 | N | SER | H | 21 | 16.361 | 11.428 | −17.959 | 1.00 | 17.59 | H |
| ATOM | 1778 | CA | SER | H | 21 | 17.210 | 11.408 | −19.144 | 1.00 | 17.64 | H |
| ATOM | 1779 | CB | SER | H | 21 | 17.582 | 12.832 | −19.555 | 1.00 | 17.12 | H |
| ATOM | 1780 | OG | SER | H | 21 | 18.339 | 13.469 | −18.545 | 1.00 | 18.60 | H |
| ATOM | 1781 | C | SER | H | 21 | 18.474 | 10.595 | −18.908 | 1.00 | 18.15 | H |
| ATOM | 1782 | O | SER | H | 21 | 18.891 | 10.369 | −17.771 | 1.00 | 19.16 | H |
| ATOM | 1783 | N | CYS | H | 22 | 19.085 | 10.168 | −20.002 | 1.00 | 19.52 | H |
| ATOM | 1784 | CA | CYS | H | 22 | 20.305 | 9.379 | −19.954 | 1.00 | 19.29 | H |
| ATOM | 1785 | C | CYS | H | 22 | 21.140 | 9.768 | −21.166 | 1.00 | 18.35 | H |
| ATOM | 1786 | O | CYS | H | 22 | 20.811 | 9.400 | −22.292 | 1.00 | 18.61 | H |
| ATOM | 1787 | CB | CYS | H | 22 | 19.934 | 7.907 | −20.014 | 1.00 | 20.40 | H |
| ATOM | 1788 | SG | CYS | H | 22 | 21.272 | 6.681 | −20.160 | 1.00 | 24.51 | H |
| ATOM | 1789 | N | LYS | H | 23 | 22.207 | 10.521 | −20.930 | 1.00 | 19.52 | H |
| ATOM | 1790 | CA | LYS | H | 23 | 23.075 | 10.973 | −22.012 | 1.00 | 21.98 | H |
| ATOM | 1791 | CB | LYS | H | 23 | 23.580 | 12.390 | −21.728 | 1.00 | 22.12 | H |
| ATOM | 1792 | CG | LYS | H | 23 | 24.442 | 12.963 | −22.845 | 1.00 | 25.90 | H |
| ATOM | 1793 | CD | LYS | H | 23 | 24.949 | 14.348 | −22.500 | 1.00 | 27.19 | H |
| ATOM | 1794 | CE | LYS | H | 23 | 25.871 | 14.878 | −23.590 | 1.00 | 31.13 | H |
| ATOM | 1795 | NZ | LYS | H | 23 | 25.185 | 14.975 | −24.911 | 1.00 | 32.12 | H |
| ATOM | 1796 | C | LYS | H | 23 | 24.262 | 10.034 | −22.181 | 1.00 | 22.53 | H |
| ATOM | 1797 | O | LYS | H | 23 | 25.010 | 9.789 | −21.237 | 1.00 | 22.77 | H |
| ATOM | 1798 | N | ALA | H | 24 | 24.433 | 9.515 | −23.389 | 1.00 | 23.09 | H |
| ATOM | 1799 | CA | ALA | H | 24 | 25.530 | 8.602 | −23.664 | 1.00 | 24.28 | H |
| ATOM | 1800 | CB | ALA | H | 24 | 25.008 | 7.375 | −24.416 | 1.00 | 21.47 | H |
| ATOM | 1801 | C | ALA | H | 24 | 26.661 | 9.257 | −24.459 | 1.00 | 26.72 | H |
| ATOM | 1802 | O | ALA | H | 24 | 26.449 | 10.217 | −25.205 | 1.00 | 26.80 | H |
| ATOM | 1803 | N | SER | H | 25 | 27.867 | 8.735 | −24.270 | 1.00 | 28.39 | H |
| ATOM | 1804 | CA | SER | H | 25 | 29.047 | 9.200 | −24.990 | 1.00 | 31.55 | H |
| ATOM | 1805 | CB | SER | H | 25 | 29.786 | 10.292 | −24.209 | 1.00 | 30.71 | H |
| ATOM | 1806 | OG | SER | H | 25 | 30.425 | 9.768 | −23.065 | 1.00 | 35.97 | H |
| ATOM | 1807 | C | SER | H | 25 | 29.930 | 7.969 | −25.156 | 1.00 | 32.24 | H |
| ATOM | 1808 | O | SER | H | 25 | 30.055 | 7.154 | −24.233 | 1.00 | 32.51 | H |
| ATOM | 1809 | N | GLY | H | 26 | 30.518 | 7.820 | −26.337 | 1.00 | 33.24 | H |
| ATOM | 1810 | CA | GLY | H | 26 | 31.360 | 6.667 | −26.606 | 1.00 | 32.73 | H |
| ATOM | 1811 | C | GLY | H | 26 | 30.557 | 5.599 | −27.330 | 1.00 | 33.76 | H |
| ATOM | 1812 | O | GLY | H | 26 | 29.331 | 5.590 | −27.256 | 1.00 | 33.10 | H |
| ATOM | 1813 | N | GLY | H | 27 | 31.236 | 4.694 | −28.026 | 1.00 | 33.84 | H |
| ATOM | 1814 | CA | GLY | H | 27 | 30.529 | 3.651 | −28.747 | 1.00 | 34.04 | H |
| ATOM | 1815 | C | GLY | H | 27 | 29.560 | 4.237 | −29.762 | 1.00 | 35.04 | H |
| ATOM | 1816 | O | GLY | H | 27 | 29.862 | 5.238 | −30.406 | 1.00 | 35.15 | H |
| ATOM | 1817 | N | SER | H | 28 | 28.394 | 3.616 | −29.910 | 1.00 | 33.81 | H |
| ATOM | 1818 | CA | SER | H | 28 | 27.394 | 4.103 | −30.851 | 1.00 | 33.89 | H |
| ATOM | 1819 | CB | SER | H | 28 | 27.340 | 3.216 | −32.093 | 1.00 | 34.14 | H |
| ATOM | 1820 | OG | SER | H | 28 | 26.257 | 3.595 | −32.922 | 1.00 | 35.77 | H |
| ATOM | 1821 | C | SER | H | 28 | 26.012 | 4.154 | −30.214 | 1.00 | 32.92 | H |
| ATOM | 1822 | O | SER | H | 28 | 25.459 | 3.130 | −29.811 | 1.00 | 33.03 | H |
| ATOM | 1823 | N | PHE | H | 29 | 25.459 | 5.358 | −30.140 | 1.00 | 29.68 | H |
| ATOM | 1824 | CA | PHE | H | 29 | 24.149 | 5.572 | −29.550 | 1.00 | 27.12 | H |
| ATOM | 1825 | CB | PHE | H | 29 | 23.880 | 7.074 | −29.402 | 1.00 | 23.91 | H |
| ATOM | 1826 | CG | PHE | H | 29 | 22.492 | 7.390 | −28.918 | 1.00 | 20.49 | H |
| ATOM | 1827 | CD1 | PHE | H | 29 | 22.128 | 7.135 | −27.594 | 1.00 | 18.09 | H |
| ATOM | 1828 | CD2 | PHE | H | 29 | 21.536 | 7.908 | −29.789 | 1.00 | 17.76 | H |
| ATOM | 1829 | CE1 | PHE | H | 29 | 20.833 | 7.389 | −27.145 | 1.00 | 17.73 | H |
| ATOM | 1830 | CE2 | PHE | H | 29 | 20.226 | 8.170 | −29.351 | 1.00 | 17.33 | H |
| ATOM | 1831 | CZ | PHE | H | 29 | 19.876 | 7.908 | −28.024 | 1.00 | 18.13 | H |
| ATOM | 1832 | C | PHE | H | 29 | 23.003 | 4.964 | −30.343 | 1.00 | 25.44 | H |
| ATOM | 1833 | O | PHE | H | 29 | 22.121 | 4.316 | −29.785 | 1.00 | 25.48 | H |
| ATOM | 1834 | N | SER | H | 30 | 23.026 | 5.182 | −31.652 | 1.00 | 25.89 | H |
| ATOM | 1835 | CA | SER | H | 30 | 21.959 | 4.724 | −32.533 | 1.00 | 26.35 | H |
| ATOM | 1836 | CB | SER | H | 30 | 22.077 | 5.439 | −33.883 | 1.00 | 28.92 | H |
| ATOM | 1837 | OG | SER | H | 30 | 22.001 | 6.847 | −33.724 | 1.00 | 33.37 | H |
| ATOM | 1838 | C | SER | H | 30 | 21.786 | 3.235 | −32.787 | 1.00 | 25.99 | H |
| ATOM | 1839 | O | SER | H | 30 | 20.655 | 2.763 | −32.913 | 1.00 | 25.27 | H |
| ATOM | 1840 | N | SER | H | 31 | 22.884 | 2.492 | −32.864 | 1.00 | 24.77 | H |
| ATOM | 1841 | CA | SER | H | 31 | 22.797 | 1.063 | −33.172 | 1.00 | 26.05 | H |
| ATOM | 1842 | CB | SER | H | 31 | 23.915 | 0.688 | −34.145 | 1.00 | 26.79 | H |
| ATOM | 1843 | OG | SER | H | 31 | 25.179 | 1.049 | −33.612 | 1.00 | 27.27 | H |
| ATOM | 1844 | C | SER | H | 31 | 22.777 | 0.054 | −32.022 | 1.00 | 24.80 | H |
| ATOM | 1845 | O | SER | H | 31 | 22.776 | −1.149 | −32.270 | 1.00 | 26.00 | H |
| ATOM | 1846 | N | TYR | H | 32 | 22.759 | 0.520 | −30.779 | 1.00 | 24.72 | H |
| ATOM | 1847 | CA | TYR | H | 32 | 22.718 | −0.400 | −29.641 | 1.00 | 24.11 | H |
| ATOM | 1848 | CB | TYR | H | 32 | 23.978 | −0.246 | −28.781 | 1.00 | 24.73 | H |
| ATOM | 1849 | CG | TYR | H | 32 | 25.206 | −0.797 | −29.464 | 1.00 | 26.51 | H |
| ATOM | 1850 | CD1 | TYR | H | 32 | 25.274 | −2.145 | −29.824 | 1.00 | 27.67 | H |
| ATOM | 1851 | CE1 | TYR | H | 32 | 26.366 | −2.653 | −30.531 | 1.00 | 29.09 | H |
| ATOM | 1852 | CD2 | TYR | H | 32 | 26.270 | 0.032 | −29.820 | 1.00 | 28.65 | H |
| ATOM | 1853 | CE2 | TYR | H | 32 | 27.370 | −0.468 | −30.528 | 1.00 | 30.73 | H |
| ATOM | 1854 | CZ | TYR | H | 32 | 27.408 | −1.811 | −30.881 | 1.00 | 30.02 | H |
| ATOM | 1855 | OH | TYR | H | 32 | 28.478 | −2.309 | −31.598 | 1.00 | 30.13 | H |
| ATOM | 1856 | C | TYR | H | 32 | 21.455 | −0.192 | −28.808 | 1.00 | 23.12 | H |
| ATOM | 1857 | O | TYR | H | 32 | 21.003 | 0.937 | −28.620 | 1.00 | 23.83 | H |
| ATOM | 1858 | N | ALA | H | 33 | 20.881 | −1.294 | −28.334 | 1.00 | 21.93 | H |
| ATOM | 1859 | CA | ALA | H | 33 | 19.654 | −1.255 | −27.548 | 1.00 | 20.44 | H |
| ATOM | 1860 | CB | ALA | H | 33 | 19.093 | −2.664 | −27.402 | 1.00 | 17.89 | H |
| ATOM | 1861 | C | ALA | H | 33 | 19.857 | −0.617 | −26.175 | 1.00 | 20.70 | H |
| ATOM | 1862 | O | ALA | H | 33 | 20.790 | −0.957 | −25.447 | 1.00 | 19.09 | H |
| ATOM | 1863 | N | ILE | H | 34 | 18.972 | 0.310 | −25.827 | 1.00 | 19.68 | H |
| ATOM | 1864 | CA | ILE | H | 34 | 19.056 | 1.005 | −24.550 | 1.00 | 20.99 | H |
| ATOM | 1865 | CB | ILE | H | 34 | 19.227 | 2.547 | −24.781 | 1.00 | 23.11 | H |
| ATOM | 1866 | CG2 | ILE | H | 34 | 18.111 | 3.064 | −25.643 | 1.00 | 28.63 | H |
| ATOM | 1867 | CG1 | ILE | H | 34 | 19.321 | 3.308 | −23.451 | 1.00 | 26.72 | H |
| ATOM | 1868 | CD1 | ILE | H | 34 | 17.990 | 3.545 | −22.750 | 1.00 | 29.23 | H |
| ATOM | 1869 | C | ILE | H | 34 | 17.801 | 0.686 | −23.746 | 1.00 | 20.52 | H |
| ATOM | 1870 | O | ILE | H | 34 | 16.685 | 0.926 | −24.198 | 1.00 | 19.71 | H |
| ATOM | 1871 | N | SER | H | 35 | 17.998 | 0.119 | −22.559 | 1.00 | 18.31 | H |
| ATOM | 1872 | CA | SER | H | 35 | 16.889 | −0.261 | −21.696 | 1.00 | 17.36 | H |
| ATOM | 1873 | CB | SER | H | 35 | 16.981 | −1.743 | −21.314 | 1.00 | 17.72 | H |
| ATOM | 1874 | OG | SER | H | 35 | 16.644 | −2.602 | −22.386 | 1.00 | 19.58 | H |
| ATOM | 1875 | C | SER | H | 35 | 16.830 | 0.539 | −20.413 | 1.00 | 16.72 | H |
| ATOM | 1876 | O | SER | H | 35 | 17.778 | 1.220 | −20.031 | 1.00 | 16.31 | H |
| ATOM | 1877 | N | TRP | H | 36 | 15.691 | 0.438 | −19.747 | 1.00 | 16.53 | H |
| ATOM | 1878 | CA | TRP | H | 36 | 15.494 | 1.099 | −18.476 | 1.00 | 14.91 | H |
| ATOM | 1879 | CB | TRP | H | 36 | 14.398 | 2.163 | −18.574 | 1.00 | 15.59 | H |
| ATOM | 1880 | CG | TRP | H | 36 | 14.844 | 3.406 | −19.282 | 1.00 | 14.70 | H |
| ATOM | 1881 | CD2 | TRP | H | 36 | 15.511 | 4.530 | −18.695 | 1.00 | 15.28 | H |
| ATOM | 1882 | CE2 | TRP | H | 36 | 15.739 | 5.467 | −19.726 | 1.00 | 13.68 | H |
| ATOM | 1883 | CE3 | TRP | H | 36 | 15.935 | 4.837 | −17.396 | 1.00 | 15.48 | H |
| ATOM | 1884 | CD1 | TRP | H | 36 | 14.703 | 3.695 | −20.607 | 1.00 | 17.00 | H |
| ATOM | 1885 | NE1 | TRP | H | 36 | 15.237 | 4.933 | −20.882 | 1.00 | 16.69 | H |
| ATOM | 1886 | CZ2 | TRP | H | 36 | 16.372 | 6.692 | −19.501 | 1.00 | 14.96 | H |
| ATOM | 1887 | CZ3 | TRP | H | 36 | 16.568 | 6.060 | −17.169 | 1.00 | 14.78 | H |
| ATOM | 1888 | CH2 | TRP | H | 36 | 16.778 | 6.971 | −18.220 | 1.00 | 15.58 | H |
| ATOM | 1889 | C | TRP | H | 36 | 15.110 | 0.018 | −17.473 | 1.00 | 13.83 | H |
| ATOM | 1890 | O | TRP | H | 36 | 14.273 | −0.836 | −17.749 | 1.00 | 13.04 | H |
| ATOM | 1891 | N | VAL | H | 37 | 15.753 | 0.057 | −16.316 | 1.00 | 12.69 | H |
| ATOM | 1892 | CA | VAL | H | 37 | 15.511 | −0.905 | −15.260 | 1.00 | 11.46 | H |
| ATOM | 1893 | CB | VAL | H | 37 | 16.694 | −1.897 | −15.146 | 1.00 | 12.05 | H |
| ATOM | 1894 | CG1 | VAL | H | 37 | 16.488 | −2.832 | −13.956 | 1.00 | 10.93 | H |
| ATOM | 1895 | CG2 | VAL | H | 37 | 16.827 | −2.697 | −16.454 | 1.00 | 8.82 | H |
| ATOM | 1896 | C | VAL | H | 37 | 15.364 | −0.151 | −13.955 | 1.00 | 12.87 | H |
| ATOM | 1897 | O | VAL | H | 37 | 16.136 | 0.768 | −13.679 | 1.00 | 12.86 | H |
| ATOM | 1898 | N | ARG | H | 38 | 14.376 | −0.525 | −13.148 | 1.00 | 11.12 | H |
| ATOM | 1899 | CA | ARG | H | 38 | 14.203 | 0.161 | −11.886 | 1.00 | 12.81 | H |
| ATOM | 1900 | CB | ARG | H | 38 | 12.856 | 0.902 | −11.836 | 1.00 | 12.72 | H |
| ATOM | 1901 | CG | ARG | H | 38 | 11.646 | 0.017 | −11.610 | 1.00 | 11.52 | H |
| ATOM | 1902 | CD | ARG | H | 38 | 10.384 | 0.853 | −11.486 | 1.00 | 10.91 | H |
| ATOM | 1903 | NE | ARG | H | 38 | 9.242 | 0.038 | −11.080 | 1.00 | 9.82 | H |
| ATOM | 1904 | CZ | ARG | H | 38 | 8.019 | 0.512 | −10.858 | 1.00 | 11.19 | H |
| ATOM | 1905 | NH1 | ARG | H | 38 | 7.052 | −0.316 | −10.492 | 1.00 | 9.02 | H |
| ATOM | 1906 | NH2 | ARG | H | 38 | 7.763 | 1.809 | −11.008 | 1.00 | 10.48 | H |
| ATOM | 1907 | C | ARG | H | 38 | 14.334 | −0.780 | −10.699 | 1.00 | 13.03 | H |
| ATOM | 1908 | O | ARG | H | 38 | 14.300 | −2.005 | −10.829 | 1.00 | 15.10 | H |
| ATOM | 1909 | N | GLN | H | 39 | 14.499 | −0.187 | −9.532 | 1.00 | 15.04 | H |
| ATOM | 1910 | CA | GLN | H | 39 | 14.658 | −0.958 | −8.324 | 1.00 | 14.14 | H |
| ATOM | 1911 | CB | GLN | H | 39 | 16.144 | −1.225 | −8.095 | 1.00 | 16.15 | H |
| ATOM | 1912 | CG | GLN | H | 39 | 16.470 | −1.994 | −6.837 | 1.00 | 13.86 | H |
| ATOM | 1913 | CD | GLN | H | 39 | 17.915 | −2.458 | −6.830 | 1.00 | 15.59 | H |
| ATOM | 1914 | OE1 | GLN | H | 39 | 18.830 | −1.656 | −7.005 | 1.00 | 13.70 | H |
| ATOM | 1915 | NE2 | GLN | H | 39 | 18.125 | −3.762 | −6.634 | 1.00 | 12.88 | H |
| ATOM | 1916 | C | GLN | H | 39 | 14.082 | −0.187 | −7.162 | 1.00 | 14.37 | H |
| ATOM | 1917 | O | GLN | H | 39 | 14.634 | 0.830 | −6.746 | 1.00 | 14.58 | H |
| ATOM | 1918 | N | ALA | H | 40 | 12.952 | −0.662 | −6.657 | 1.00 | 14.98 | H |
| ATOM | 1919 | CA | ALA | H | 40 | 12.318 | −0.028 | −5.514 | 1.00 | 15.23 | H |
| ATOM | 1920 | CB | ALA | H | 40 | 10.920 | −0.602 | −5.301 | 1.00 | 14.46 | H |
| ATOM | 1921 | C | ALA | H | 40 | 13.209 | −0.326 | −4.309 | 1.00 | 17.48 | H |
| ATOM | 1922 | O | ALA | H | 40 | 13.986 | −1.284 | −4.323 | 1.00 | 16.41 | H |
| ATOM | 1923 | N | PRO | H | 41 | 13.106 | 0.489 | −3.248 | 1.00 | 19.70 | H |
| ATOM | 1924 | CD | PRO | H | 41 | 12.204 | 1.642 | −3.095 | 1.00 | 19.39 | H |
| ATOM | 1925 | CA | PRO | H | 41 | 13.916 | 0.302 | −2.042 | 1.00 | 21.53 | H |
| ATOM | 1926 | CB | PRO | H | 41 | 13.345 | 1.341 | −1.080 | 1.00 | 22.88 | H |
| ATOM | 1927 | CG | PRO | H | 41 | 12.890 | 2.436 | −2.010 | 1.00 | 22.89 | H |
| ATOM | 1928 | C | PRO | H | 41 | 13.819 | −1.107 | −1.481 | 1.00 | 22.10 | H |
| ATOM | 1929 | O | PRO | H | 41 | 12.721 | −1.612 | −1.244 | 1.00 | 21.85 | H |
| ATOM | 1930 | N | GLY | H | 42 | 14.974 | −1.736 | −1.281 | 1.00 | 22.29 | H |
| ATOM | 1931 | CA | GLY | H | 42 | 15.004 | −3.082 | −0.733 | 1.00 | 22.34 | H |
| ATOM | 1932 | C | GLY | H | 42 | 14.498 | −4.191 | −1.639 | 1.00 | 22.90 | H |
| ATOM | 1933 | O | GLY | H | 42 | 14.442 | −5.344 | −1.221 | 1.00 | 23.75 | H |
| ATOM | 1934 | N | GLN | H | 43 | 14.134 | −3.861 | −2.875 | 1.00 | 23.81 | H |
| ATOM | 1935 | CA | GLN | H | 43 | 13.631 | −4.873 | −3.809 | 1.00 | 23.01 | H |
| ATOM | 1936 | CB | GLN | H | 43 | 12.285 | −4.431 | −4.396 | 1.00 | 27.57 | H |
| ATOM | 1937 | CG | GLN | H | 43 | 11.167 | −4.321 | −3.375 | 1.00 | 35.08 | H |
| ATOM | 1938 | CD | GLN | H | 43 | 10.076 | −5.350 | −3.605 | 1.00 | 43.06 | H |
| ATOM | 1939 | OE1 | GLN | H | 43 | 10.326 | −6.558 | −3.567 | 1.00 | 45.82 | H |
| ATOM | 1940 | NE2 | GLN | H | 43 | 8.856 | −4.875 | −3.849 | 1.00 | 45.40 | H |
| ATOM | 1941 | C | GLN | H | 43 | 14.609 | −5.165 | −4.946 | 1.00 | 20.18 | H |
| ATOM | 1942 | O | GLN | H | 43 | 15.659 | −4.534 | −5.056 | 1.00 | 16.85 | H |
| ATOM | 1943 | N | GLY | H | 44 | 14.239 | −6.120 | −5.796 | 1.00 | 17.59 | H |
| ATOM | 1944 | CA | GLY | H | 44 | 15.086 | −6.508 | −6.908 | 1.00 | 16.06 | H |
| ATOM | 1945 | C | GLY | H | 44 | 14.966 | −5.641 | −8.146 | 1.00 | 16.74 | H |
| ATOM | 1946 | O | GLY | H | 44 | 14.226 | −4.665 | −8.175 | 1.00 | 20.38 | H |
| ATOM | 1947 | N | LEU | H | 45 | 15.715 | −6.001 | −9.176 | 1.00 | 14.76 | H |
| ATOM | 1948 | CA | LEU | H | 45 | 15.713 | −5.269 | −10.436 | 1.00 | 14.05 | H |
| ATOM | 1949 | CB | LEU | H | 45 | 16.977 | −5.601 | −11.229 | 1.00 | 11.20 | H |
| ATOM | 1950 | CG | LEU | H | 45 | 18.286 | −5.363 | −10.476 | 1.00 | 11.37 | H |
| ATOM | 1951 | CD1 | LEU | H | 45 | 19.446 | −5.965 | −11.249 | 1.00 | 8.84 | H |
| ATOM | 1952 | CD2 | LEU | H | 45 | 18.470 | −3.861 | −10.255 | 1.00 | 7.91 | H |
| ATOM | 1953 | C | LEU | H | 45 | 14.504 | −5.640 | −11.282 | 1.00 | 15.73 | H |
| ATOM | 1954 | O | LEU | H | 45 | 14.017 | −6.770 | −11.227 | 1.00 | 14.18 | H |
| ATOM | 1955 | N | GLU | H | 46 | 14.020 | −4.683 | −12.067 | 1.00 | 16.26 | H |
| ATOM | 1956 | CA | GLU | H | 46 | 12.895 | −4.946 | −12.941 | 1.00 | 15.29 | H |
| ATOM | 1957 | CB | GLU | H | 46 | 11.579 | −4.518 | −12.290 | 1.00 | 18.06 | H |
| ATOM | 1958 | CG | GLU | H | 46 | 10.363 | −4.960 | −13.104 | 1.00 | 20.71 | H |
| ATOM | 1959 | CD | GLU | H | 46 | 9.039 | −4.638 | −12.440 | 1.00 | 23.61 | H |
| ATOM | 1960 | OE1 | GLU | H | 46 | 8.008 | −5.155 | −12.912 | 1.00 | 26.51 | H |
| ATOM | 1961 | OE2 | GLU | H | 46 | 9.019 | −3.873 | −11.457 | 1.00 | 24.93 | H |
| ATOM | 1962 | C | GLU | H | 46 | 13.069 | −4.221 | −14.268 | 1.00 | 15.04 | H |
| ATOM | 1963 | O | GLU | H | 46 | 13.190 | −2.985 | −14.312 | 1.00 | 13.75 | H |
| ATOM | 1964 | N | TRP | H | 47 | 13.106 | −5.001 | −15.343 | 1.00 | 12.40 | H |
| ATOM | 1965 | CA | TRP | H | 47 | 13.248 | −4.454 | −16.684 | 1.00 | 13.91 | H |
| ATOM | 1966 | CB | TRP | H | 47 | 13.545 | −5.572 | −17.688 | 1.00 | 13.87 | H |
| ATOM | 1967 | CG | TRP | H | 47 | 13.678 | −5.095 | −19.104 | 1.00 | 12.71 | H |
| ATOM | 1968 | CD2 | TRP | H | 47 | 12.720 | −5.259 | −20.158 | 1.00 | 12.29 | H |
| ATOM | 1969 | CE2 | TRP | H | 47 | 13.256 | −4.644 | −21.313 | 1.00 | 13.40 | H |
| ATOM | 1970 | CE3 | TRP | H | 47 | 11.458 | −5.866 | −20.239 | 1.00 | 13.01 | H |
| ATOM | 1971 | CD1 | TRP | H | 47 | 14.729 | −4.406 | −19.646 | 1.00 | 12.43 | H |
| ATOM | 1972 | NE1 | TRP | H | 47 | 14.481 | −4.132 | −20.974 | 1.00 | 14.24 | H |
| ATOM | 1973 | CZ2 | TRP | H | 47 | 12.576 | −4.622 | −22.537 | 1.00 | 11.52 | H |
| ATOM | 1974 | CZ3 | TRP | H | 47 | 10.781 | −5.844 | −21.457 | 1.00 | 13.58 | H |
| ATOM | 1975 | CH2 | TRP | H | 47 | 11.345 | −5.225 | −22.589 | 1.00 | 12.86 | H |
| ATOM | 1976 | C | TRP | H | 47 | 11.917 | −3.797 | −17.020 | 1.00 | 15.52 | H |
| ATOM | 1977 | O | TRP | H | 47 | 10.868 | −4.428 | −16.914 | 1.00 | 14.29 | H |
| ATOM | 1978 | N | MET | H | 48 | 11.958 | −2.530 | −17.413 | 1.00 | 16.81 | H |
| ATOM | 1979 | CA | MET | H | 48 | 10.736 | −1.802 | −17.750 | 1.00 | 16.50 | H |
| ATOM | 1980 | CB | MET | H | 48 | 10.850 | −0.355 | −17.273 | 1.00 | 15.66 | H |
| ATOM | 1981 | CG | MET | H | 48 | 11.071 | −0.217 | −15.768 | 1.00 | 17.20 | H |
| ATOM | 1982 | SD | MET | H | 48 | 11.034 | 1.507 | −15.240 | 1.00 | 17.91 | H |
| ATOM | 1983 | CE | MET | H | 48 | 9.327 | 1.914 | −15.620 | 1.00 | 22.20 | H |
| ATOM | 1984 | C | MET | H | 48 | 10.503 | −1.837 | −19.253 | 1.00 | 16.70 | H |
| ATOM | 1985 | O | MET | H | 48 | 9.376 | −2.005 | −19.724 | 1.00 | 17.49 | H |
| ATOM | 1986 | N | GLY | H | 49 | 11.588 | −1.679 | −20.000 | 1.00 | 16.28 | H |
| ATOM | 1987 | CA | GLY | H | 49 | 11.511 | −1.693 | −21.446 | 1.00 | 14.49 | H |
| ATOM | 1988 | C | GLY | H | 49 | 12.771 | −1.102 | −22.037 | 1.00 | 14.12 | H |
| ATOM | 1989 | O | GLY | H | 49 | 13.714 | −0.778 | −21.309 | 1.00 | 15.17 | H |
| ATOM | 1990 | N | GLY | H | 50 | 12.800 | −0.963 | −23.358 | 1.00 | 13.77 | H |
| ATOM | 1991 | CA | GLY | H | 50 | 13.970 | −0.396 | −23.995 | 1.00 | 14.00 | H |
| ATOM | 1992 | C | GLY | H | 50 | 13.680 | 0.165 | −25.373 | 1.00 | 18.11 | H |
| ATOM | 1993 | O | GLY | H | 50 | 12.546 | 0.121 | −25.852 | 1.00 | 17.14 | H |
| ATOM | 1994 | N | ILE | H | 51 | 14.711 | 0.700 | −26.015 | 1.00 | 18.01 | H |
| ATOM | 1995 | CA | ILE | H | 51 | 14.551 | 1.247 | −27.347 | 1.00 | 19.29 | H |
| ATOM | 1996 | CB | ILE | H | 51 | 14.150 | 2.747 | −27.298 | 1.00 | 20.46 | H |
| ATOM | 1997 | CG2 | ILE | H | 51 | 15.237 | 3.558 | −26.618 | 1.00 | 17.92 | H |
| ATOM | 1998 | CG1 | ILE | H | 51 | 13.938 | 3.279 | −28.718 | 1.00 | 22.44 | H |
| ATOM | 1999 | CD1 | ILE | H | 51 | 13.472 | 4.713 | −28.764 | 1.00 | 29.45 | H |
| ATOM | 2000 | C | ILE | H | 51 | 15.824 | 1.120 | −28.176 | 1.00 | 20.01 | H |
| ATOM | 2001 | O | ILE | H | 51 | 16.934 | 1.157 | −27.642 | 1.00 | 19.35 | H |
| ATOM | 2002 | N | ILE | H | 52 | 15.640 | 0.939 | −29.479 | 1.00 | 21.19 | H |
| ATOM | 2003 | CA | ILE | H | 52 | 16.740 | 0.874 | −30.441 | 1.00 | 21.29 | H |
| ATOM | 2004 | CB | ILE | H | 52 | 16.605 | −0.330 | −31.391 | 1.00 | 22.07 | H |
| ATOM | 2005 | CG2 | ILE | H | 52 | 17.691 | −0.265 | −32.474 | 1.00 | 19.34 | H |
| ATOM | 2006 | CG1 | ILE | H | 52 | 16.701 | −1.628 | −30.588 | 1.00 | 18.91 | H |
| ATOM | 2007 | CD1 | ILE | H | 52 | 16.418 | −2.868 | −31.393 | 1.00 | 21.10 | H |
| ATOM | 2008 | C | ILE | H | 52 | 16.510 | 2.162 | −31.224 | 1.00 | 21.55 | H |
| ATOM | 2009 | O | ILE | H | 52 | 15.596 | 2.243 | −32.036 | 1.00 | 19.40 | H |
| ATOM | 2010 | N | PRO | H | 52A | 17.329 | 3.194 | −30.972 | 1.00 | 23.09 | H |
| ATOM | 2011 | CD | PRO | H | 52A | 18.490 | 3.201 | −30.067 | 1.00 | 21.84 | H |
| ATOM | 2012 | CA | PRO | H | 52A | 17.194 | 4.487 | −31.649 | 1.00 | 24.13 | H |
| ATOM | 2013 | CB | PRO | H | 52A | 18.421 | 5.259 | −31.166 | 1.00 | 23.29 | H |
| ATOM | 2014 | CG | PRO | H | 52A | 18.674 | 4.676 | −29.812 | 1.00 | 23.21 | H |
| ATOM | 2015 | C | PRO | H | 52A | 17.072 | 4.483 | −33.171 | 1.00 | 26.22 | H |
| ATOM | 2016 | O | PRO | H | 52A | 16.119 | 5.035 | −33.713 | 1.00 | 25.25 | H |
| ATOM | 2017 | N | SER | H | 53 | 18.026 | 3.857 | −33.852 | 1.00 | 29.61 | H |
| ATOM | 2018 | CA | SER | H | 53 | 18.042 | 3.818 | −35.312 | 1.00 | 33.24 | H |
| ATOM | 2019 | CB | SER | H | 53 | 18.918 | 2.665 | −35.807 | 1.00 | 34.50 | H |
| ATOM | 2020 | OG | SER | H | 53 | 19.249 | 2.840 | −37.177 | 1.00 | 39.10 | H |
| ATOM | 2021 | C | SER | H | 53 | 16.672 | 3.741 | −35.979 | 1.00 | 34.60 | H |
| ATOM | 2022 | O | SER | H | 53 | 16.378 | 4.527 | −36.875 | 1.00 | 36.82 | H |
| ATOM | 2023 | N | ASP | H | 54 | 15.827 | 2.811 | −35.552 | 1.00 | 35.84 | H |
| ATOM | 2024 | CA | ASP | H | 54 | 14.502 | 2.696 | −36.160 | 1.00 | 35.39 | H |
| ATOM | 2025 | CB | ASP | H | 54 | 14.301 | 1.296 | −36.738 | 1.00 | 39.17 | H |
| ATOM | 2026 | CG | ASP | H | 54 | 14.970 | 1.123 | −38.080 | 1.00 | 42.15 | H |
| ATOM | 2027 | OD1 | ASP | H | 54 | 15.832 | 0.225 | −38.205 | 1.00 | 44.09 | H |
| ATOM | 2028 | OD2 | ASP | H | 54 | 14.629 | 1.886 | −39.009 | 1.00 | 43.91 | H |
| ATOM | 2029 | C | ASP | H | 54 | 13.364 | 3.008 | −35.206 | 1.00 | 32.99 | H |
| ATOM | 2030 | O | ASP | H | 54 | 12.203 | 2.732 | −35.508 | 1.00 | 31.89 | H |
| ATOM | 2031 | N | SER | H | 55 | 13.697 | 3.588 | −34.058 | 1.00 | 30.90 | H |
| ATOM | 2032 | CA | SER | H | 55 | 12.692 | 3.927 | −33.061 | 1.00 | 29.46 | H |
| ATOM | 2033 | CB | SER | H | 55 | 11.746 | 4.997 | −33.612 | 1.00 | 33.38 | H |
| ATOM | 2034 | OG | SER | H | 55 | 12.457 | 6.178 | −33.938 | 1.00 | 36.26 | H |
| ATOM | 2035 | C | SER | H | 55 | 11.892 | 2.691 | −32.661 | 1.00 | 25.93 | H |
| ATOM | 2036 | O | SER | H | 55 | 10.677 | 2.756 | −32.484 | 1.00 | 24.90 | H |
| ATOM | 2037 | N | THR | H | 56 | 12.575 | 1.560 | −32.529 | 1.00 | 23.60 | H |
| ATOM | 2038 | CA | THR | H | 56 | 11.899 | 0.332 | −32.141 | 1.00 | 22.84 | H |
| ATOM | 2039 | CB | THR | H | 56 | 12.637 | −0.911 | −32.685 | 1.00 | 23.67 | H |
| ATOM | 2040 | OG1 | THR | H | 56 | 12.826 | −0.782 | −34.101 | 1.00 | 27.56 | H |
| ATOM | 2041 | CG2 | THR | H | 56 | 11.826 | −2.172 | −32.406 | 1.00 | 20.88 | H |
| ATOM | 2042 | C | THR | H | 56 | 11.819 | 0.241 | −30.614 | 1.00 | 22.26 | H |
| ATOM | 2043 | O | THR | H | 56 | 12.828 | 0.028 | −29.939 | 1.00 | 21.53 | H |
| ATOM | 2044 | N | THR | H | 57 | 10.618 | 0.423 | −30.076 | 1.00 | 21.70 | H |
| ATOM | 2045 | CA | THR | H | 57 | 10.419 | 0.345 | −28.639 | 1.00 | 21.72 | H |
| ATOM | 2046 | CB | THR | H | 57 | 9.416 | 1.402 | −28.129 | 1.00 | 21.24 | H |
| ATOM | 2047 | OG1 | THR | H | 57 | 8.210 | 1.333 | −28.898 | 1.00 | 23.23 | H |
| ATOM | 2048 | CG2 | THR | H | 57 | 10.018 | 2.794 | −28.226 | 1.00 | 21.43 | H |
| ATOM | 2049 | C | THR | H | 57 | 9.904 | −1.031 | −28.277 | 1.00 | 22.01 | H |
| ATOM | 2050 | O | THR | H | 57 | 9.322 | −1.722 | −29.107 | 1.00 | 22.98 | H |
| ATOM | 2051 | N | ASN | H | 58 | 10.124 | −1.418 | −27.027 | 1.00 | 20.80 | H |
| ATOM | 2052 | CA | ASN | H | 58 | 9.713 | −2.721 | −26.520 | 1.00 | 19.87 | H |
| ATOM | 2053 | CB | ASN | H | 58 | 10.864 | −3.718 | −26.730 | 1.00 | 18.39 | H |
| ATOM | 2054 | CG | ASN | H | 58 | 10.630 | −5.054 | −26.050 | 1.00 | 16.89 | H |
| ATOM | 2055 | OD1 | ASN | H | 58 | 11.574 | −5.818 | −25.831 | 1.00 | 21.07 | H |
| ATOM | 2056 | ND2 | ASN | H | 58 | 9.388 | −5.348 | −25.726 | 1.00 | 11.36 | H |
| ATOM | 2057 | C | ASN | H | 58 | 9.441 | −2.528 | −25.036 | 1.00 | 18.33 | H |
| ATOM | 2058 | O | ASN | H | 58 | 10.370 | −2.359 | −24.259 | 1.00 | 19.83 | H |
| ATOM | 2059 | N | TYR | H | 59 | 8.173 | −2.552 | −24.640 | 1.00 | 18.91 | H |
| ATOM | 2060 | CA | TYR | H | 59 | 7.826 | −2.347 | −23.235 | 1.00 | 17.48 | H |
| ATOM | 2061 | CB | TYR | H | 59 | 6.726 | −1.288 | −23.086 | 1.00 | 16.64 | H |
| ATOM | 2062 | CG | TYR | H | 59 | 6.991 | 0.031 | −23.781 | 1.00 | 18.59 | H |
| ATOM | 2063 | CD1 | TYR | H | 59 | 8.247 | 0.639 | −23.725 | 1.00 | 17.63 | H |
| ATOM | 2064 | CE1 | TYR | H | 59 | 8.476 | 1.869 | −24.336 | 1.00 | 17.36 | H |
| ATOM | 2065 | CD2 | TYR | H | 59 | 5.968 | 0.692 | −24.468 | 1.00 | 18.53 | H |
| ATOM | 2066 | CE2 | TYR | H | 59 | 6.187 | 1.926 | −25.080 | 1.00 | 16.03 | H |
| ATOM | 2067 | CZ | TYR | H | 59 | 7.442 | 2.505 | −25.010 | 1.00 | 18.32 | H |
| ATOM | 2068 | OH | TYR | H | 59 | 7.672 | 3.715 | −25.615 | 1.00 | 14.86 | H |
| ATOM | 2069 | C | TYR | H | 59 | 7.353 | −3.596 | −22.517 | 1.00 | 15.52 | H |
| ATOM | 2070 | O | TYR | H | 59 | 6.757 | −4.483 | −23.118 | 1.00 | 13.44 | H |
| ATOM | 2071 | N | ALA | H | 60 | 7.613 | −3.647 | −21.215 | 1.00 | 15.61 | H |
| ATOM | 2072 | CA | ALA | H | 60 | 7.161 | −4.764 | −20.400 | 1.00 | 15.20 | H |
| ATOM | 2073 | CB | ALA | H | 60 | 7.843 | −4.743 | −19.037 | 1.00 | 14.83 | H |
| ATOM | 2074 | C | ALA | H | 60 | 5.656 | −4.561 | −20.238 | 1.00 | 15.96 | H |
| ATOM | 2075 | O | ALA | H | 60 | 5.193 | −3.439 | −20.039 | 1.00 | 16.26 | H |
| ATOM | 2076 | N | PRO | H | 61 | 4.873 | −5.643 | −20.323 | 1.00 | 17.82 | H |
| ATOM | 2077 | CD | PRO | H | 61 | 5.303 | −7.037 | −20.534 | 1.00 | 19.10 | H |
| ATOM | 2078 | CA | PRO | H | 61 | 3.415 | −5.557 | −20.185 | 1.00 | 18.39 | H |
| ATOM | 2079 | CB | PRO | H | 61 | 2.995 | −7.021 | −20.080 | 1.00 | 19.72 | H |
| ATOM | 2080 | CG | PRO | H | 61 | 4.013 | −7.710 | −20.958 | 1.00 | 22.03 | H |
| ATOM | 2081 | C | PRO | H | 61 | 2.960 | −4.743 | −18.978 | 1.00 | 19.93 | H |
| ATOM | 2082 | O | PRO | H | 61 | 2.050 | −3.926 | −19.082 | 1.00 | 22.98 | H |
| ATOM | 2083 | N | SER | H | 62 | 3.597 | −4.953 | −17.832 | 1.00 | 20.79 | H |
| ATOM | 2084 | CA | SER | H | 62 | 3.203 | −4.231 | −16.630 | 1.00 | 22.83 | H |
| ATOM | 2085 | CB | SER | H | 62 | 3.966 | −4.752 | −15.407 | 1.00 | 24.35 | H |
| ATOM | 2086 | OG | SER | H | 62 | 5.349 | −4.497 | −15.516 | 1.00 | 32.31 | H |
| ATOM | 2087 | C | SER | H | 62 | 3.377 | −2.721 | −16.748 | 1.00 | 22.34 | H |
| ATOM | 2088 | O | SER | H | 62 | 2.874 | −1.984 | −15.920 | 1.00 | 21.39 | H |
| ATOM | 2089 | N | PHE | H | 63 | 4.075 | −2.254 | −17.777 | 1.00 | 21.17 | H |
| ATOM | 2090 | CA | PHE | H | 63 | 4.262 | −0.821 | −17.946 | 1.00 | 20.06 | H |
| ATOM | 2091 | CB | PHE | H | 63 | 5.756 | −0.474 | −17.901 | 1.00 | 20.30 | H |
| ATOM | 2092 | CG | PHE | H | 63 | 6.391 | −0.729 | −16.560 | 1.00 | 20.38 | H |
| ATOM | 2093 | CD1 | PHE | H | 63 | 6.023 | 0.026 | −15.444 | 1.00 | 20.76 | H |
| ATOM | 2094 | CD2 | PHE | H | 63 | 7.300 | −1.765 | −16.394 | 1.00 | 16.91 | H |
| ATOM | 2095 | CE1 | PHE | H | 63 | 6.548 | −0.254 | −14.181 | 1.00 | 18.92 | H |
| ATOM | 2096 | CE2 | PHE | H | 63 | 7.831 | −2.056 | −15.140 | 1.00 | 18.74 | H |
| ATOM | 2097 | CZ | PHE | H | 63 | 7.453 | −1.300 | −14.029 | 1.00 | 19.77 | H |
| ATOM | 2098 | C | PHE | H | 63 | 3.626 | −0.312 | −19.242 | 1.00 | 20.59 | H |
| ATOM | 2099 | O | PHE | H | 63 | 3.575 | 0.893 | −19.489 | 1.00 | 18.77 | H |
| ATOM | 2100 | N | GLN | H | 64 | 3.136 | −1.236 | −20.063 | 1.00 | 20.72 | H |
| ATOM | 2101 | CA | GLN | H | 64 | 2.495 | −0.868 | −21.319 | 1.00 | 22.24 | H |
| ATOM | 2102 | CB | GLN | H | 64 | 2.094 | −2.116 | −22.098 | 1.00 | 22.85 | H |
| ATOM | 2103 | CG | GLN | H | 64 | 1.431 | −1.815 | −23.429 | 1.00 | 27.00 | H |
| ATOM | 2104 | CD | GLN | H | 64 | 2.432 | −1.609 | −24.546 | 1.00 | 31.26 | H |
| ATOM | 2105 | OE1 | GLN | H | 64 | 3.129 | −2.547 | −24.948 | 1.00 | 30.76 | H |
| ATOM | 2106 | NE2 | GLN | H | 64 | 2.514 | −0.382 | −25.055 | 1.00 | 32.64 | H |
| ATOM | 2107 | C | GLN | H | 64 | 1.244 | −0.047 | −20.997 | 1.00 | 21.34 | H |
| ATOM | 2108 | O | GLN | H | 64 | 0.289 | −0.561 | −20.423 | 1.00 | 20.88 | H |
| ATOM | 2109 | N | GLY | H | 65 | 1.262 | 1.229 | −21.367 | 1.00 | 20.90 | H |
| ATOM | 2110 | CA | GLY | H | 65 | 0.132 | 2.093 | −21.087 | 1.00 | 19.95 | H |
| ATOM | 2111 | C | GLY | H | 65 | 0.515 | 3.243 | −20.173 | 1.00 | 20.11 | H |
| ATOM | 2112 | O | GLY | H | 65 | −0.211 | 4.230 | −20.074 | 1.00 | 19.54 | H |
| ATOM | 2113 | N | ARG | H | 66 | 1.659 | 3.127 | −19.505 | 1.00 | 18.14 | H |
| ATOM | 2114 | CA | ARG | H | 66 | 2.114 | 4.177 | −18.598 | 1.00 | 18.53 | H |
| ATOM | 2115 | CB | ARG | H | 66 | 2.163 | 3.661 | −17.159 | 1.00 | 20.15 | H |
| ATOM | 2116 | CG | ARG | H | 66 | 0.865 | 3.701 | −16.388 | 1.00 | 21.72 | H |
| ATOM | 2117 | CD | ARG | H | 66 | 1.162 | 3.993 | −14.922 | 1.00 | 21.90 | H |
| ATOM | 2118 | NE | ARG | H | 66 | 2.050 | 2.996 | −14.326 | 1.00 | 20.20 | H |
| ATOM | 2119 | CZ | ARG | H | 66 | 2.738 | 3.186 | −13.206 | 1.00 | 18.63 | H |
| ATOM | 2120 | NH1 | ARG | H | 66 | 3.519 | 2.224 | −12.727 | 1.00 | 19.92 | H |
| ATOM | 2121 | NH2 | ARG | H | 66 | 2.661 | 4.346 | −12.574 | 1.00 | 19.84 | H |
| ATOM | 2122 | C | ARG | H | 66 | 3.508 | 4.662 | −18.949 | 1.00 | 17.71 | H |
| ATOM | 2123 | O | ARG | H | 66 | 3.994 | 5.639 | −18.388 | 1.00 | 19.62 | H |
| ATOM | 2124 | N | ILE | H | 67 | 4.147 | 3.985 | −19.889 | 1.00 | 17.92 | H |
| ATOM | 2125 | CA | ILE | H | 67 | 5.520 | 4.303 | −20.223 | 1.00 | 16.13 | H |
| ATOM | 2126 | CB | ILE | H | 67 | 6.400 | 3.067 | −19.871 | 1.00 | 19.04 | H |
| ATOM | 2127 | CG2 | ILE | H | 67 | 5.995 | 1.888 | −20.733 | 1.00 | 18.82 | H |
| ATOM | 2128 | CG1 | ILE | H | 67 | 7.878 | 3.344 | −20.094 | 1.00 | 19.95 | H |
| ATOM | 2129 | CD1 | ILE | H | 67 | 8.743 | 2.144 | −19.752 | 1.00 | 21.31 | H |
| ATOM | 2130 | C | ILE | H | 67 | 5.776 | 4.718 | −21.666 | 1.00 | 15.61 | H |
| ATOM | 2131 | O | ILE | H | 67 | 5.107 | 4.264 | −22.597 | 1.00 | 15.68 | H |
| ATOM | 2132 | N | THR | H | 68 | 6.746 | 5.605 | −21.831 | 1.00 | 14.95 | H |
| ATOM | 2133 | CA | THR | H | 68 | 7.164 | 6.056 | −23.142 | 1.00 | 17.72 | H |
| ATOM | 2134 | CB | THR | H | 68 | 6.566 | 7.438 | −23.526 | 1.00 | 18.38 | H |
| ATOM | 2135 | OG1 | THR | H | 68 | 5.135 | 7.355 | −23.567 | 1.00 | 22.62 | H |
| ATOM | 2136 | CG2 | THR | H | 68 | 7.064 | 7.850 | −24.909 | 1.00 | 18.84 | H |
| ATOM | 2137 | C | THR | H | 68 | 8.684 | 6.170 | −23.095 | 1.00 | 16.12 | H |
| ATOM | 2138 | O | THR | H | 68 | 9.246 | 6.783 | −22.194 | 1.00 | 17.75 | H |
| ATOM | 2139 | N | ILE | H | 69 | 9.349 | 5.552 | −24.056 | 1.00 | 18.02 | H |
| ATOM | 2140 | CA | ILE | H | 69 | 10.801 | 5.610 | −24.116 | 1.00 | 17.26 | H |
| ATOM | 2141 | CB | ILE | H | 69 | 11.419 | 4.199 | −24.006 | 1.00 | 14.20 | H |
| ATOM | 2142 | CG2 | ILE | H | 69 | 12.924 | 4.274 | −24.223 | 1.00 | 14.07 | H |
| ATOM | 2143 | CG1 | ILE | H | 69 | 11.093 | 3.600 | −22.631 | 1.00 | 13.69 | H |
| ATOM | 2144 | CD1 | ILE | H | 69 | 11.536 | 2.146 | −22.461 | 1.00 | 11.59 | H |
| ATOM | 2145 | C | ILE | H | 69 | 11.170 | 6.238 | −25.455 | 1.00 | 18.40 | H |
| ATOM | 2146 | O | ILE | H | 69 | 10.725 | 5.779 | −26.503 | 1.00 | 21.75 | H |
| ATOM | 2147 | N | SER | H | 70 | 11.972 | 7.296 | −25.411 | 1.00 | 19.93 | H |
| ATOM | 2148 | CA | SER | H | 70 | 12.385 | 7.988 | −26.627 | 1.00 | 20.98 | H |
| ATOM | 2149 | CB | SER | H | 70 | 11.692 | 9.349 | −26.725 | 1.00 | 20.64 | H |
| ATOM | 2150 | OG | SER | H | 70 | 11.851 | 10.069 | −25.520 | 0.60 | 18.70 | H |
| ATOM | 2151 | C | SER | H | 70 | 13.891 | 8.194 | −26.667 | 1.00 | 21.10 | H |
| ATOM | 2152 | O | SER | H | 70 | 14.561 | 8.204 | −25.631 | 1.00 | 21.33 | H |
| ATOM | 2153 | N | ALA | H | 71 | 14.419 | 8.367 | −27.872 | 1.00 | 21.16 | H |
| ATOM | 2154 | CA | ALA | H | 71 | 15.847 | 8.579 | −28.051 | 1.00 | 23.82 | H |
| ATOM | 2155 | CB | ALA | H | 71 | 16.498 | 7.317 | −28.591 | 1.00 | 20.43 | H |
| ATOM | 2156 | C | ALA | H | 71 | 16.098 | 9.746 | −28.994 | 1.00 | 24.26 | H |
| ATOM | 2157 | O | ALA | H | 71 | 15.514 | 9.827 | −30.072 | 1.00 | 26.33 | H |
| ATOM | 2158 | N | ASP | H | 72 | 16.973 | 10.649 | −28.574 | 1.00 | 26.00 | H |
| ATOM | 2159 | CA | ASP | H | 72 | 17.308 | 11.824 | −29.366 | 1.00 | 28.13 | H |
| ATOM | 2160 | CB | ASP | H | 72 | 17.291 | 13.062 | −28.466 | 1.00 | 28.26 | H |
| ATOM | 2161 | CG | ASP | H | 72 | 17.420 | 14.363 | −29.246 | 1.00 | 30.14 | H |
| ATOM | 2162 | OD1 | ASP | H | 72 | 18.177 | 14.406 | −30.237 | 1.00 | 26.71 | H |
| ATOM | 2163 | OD2 | ASP | H | 72 | 16.772 | 15.352 | −28.850 | 1.00 | 32.01 | H |
| ATOM | 2164 | C | ASP | H | 72 | 18.694 | 11.649 | −29.992 | 1.00 | 26.88 | H |
| ATOM | 2165 | O | ASP | H | 72 | 19.705 | 11.960 | −29.367 | 1.00 | 29.75 | H |
| ATOM | 2166 | N | ASN | H | 73 | 18.727 | 11.151 | −31.223 | 1.00 | 27.16 | H |
| ATOM | 2167 | CA | ASN | H | 73 | 19.981 | 10.925 | −31.947 | 1.00 | 28.60 | H |
| ATOM | 2168 | CB | ASN | H | 73 | 19.691 | 10.497 | −33.395 | 1.00 | 31.33 | H |
| ATOM | 2169 | CG | ASN | H | 73 | 19.129 | 9.086 | −33.498 | 1.00 | 35.82 | H |
| ATOM | 2170 | OD1 | ASN | H | 73 | 18.278 | 8.686 | −32.704 | 1.00 | 40.73 | H |
| ATOM | 2171 | ND2 | ASN | H | 73 | 19.589 | 8.330 | −34.498 | 1.00 | 35.15 | H |
| ATOM | 2172 | C | ASN | H | 73 | 20.925 | 12.130 | −31.975 | 1.00 | 28.03 | H |
| ATOM | 2173 | O | ASN | H | 73 | 22.143 | 11.963 | −31.928 | 1.00 | 28.87 | H |
| ATOM | 2174 | N | SER | H | 74 | 20.378 | 13.340 | −32.047 | 1.00 | 26.58 | H |
| ATOM | 2175 | CA | SER | H | 74 | 21.227 | 14.527 | −32.106 | 1.00 | 27.35 | H |
| ATOM | 2176 | CB | SER | H | 74 | 20.411 | 15.758 | −32.522 | 1.00 | 27.62 | H |
| ATOM | 2177 | OG | SER | H | 74 | 19.485 | 16.139 | −31.524 | 1.00 | 30.05 | H |
| ATOM | 2178 | C | SER | H | 74 | 21.992 | 14.827 | −30.818 | 1.00 | 27.08 | H |
| ATOM | 2179 | O | SER | H | 74 | 22.996 | 15.539 | −30.847 | 1.00 | 27.61 | H |
| ATOM | 2180 | N | THR | H | 75 | 21.533 | 14.300 | −29.687 | 1.00 | 25.43 | H |
| ATOM | 2181 | CA | THR | H | 75 | 22.246 | 14.545 | −28.434 | 1.00 | 25.07 | H |
| ATOM | 2182 | CB | THR | H | 75 | 21.394 | 15.338 | −27.413 | 1.00 | 25.27 | H |
| ATOM | 2183 | OG1 | THR | H | 75 | 20.191 | 14.616 | −27.131 | 1.00 | 25.53 | H |
| ATOM | 2184 | CG2 | THR | H | 75 | 21.054 | 16.729 | −27.953 | 1.00 | 22.11 | H |
| ATOM | 2185 | C | THR | H | 75 | 22.679 | 13.251 | −27.765 | 1.00 | 24.88 | H |
| ATOM | 2186 | O | THR | H | 75 | 23.264 | 13.280 | −26.691 | 1.00 | 26.55 | H |
| ATOM | 2187 | N | ASN | H | 76 | 22.400 | 12.120 | −28.403 | 1.00 | 23.75 | H |
| ATOM | 2188 | CA | ASN | H | 76 | 22.762 | 10.835 | −27.819 | 1.00 | 25.04 | H |
| ATOM | 2189 | CB | ASN | H | 76 | 24.283 | 10.706 | −27.718 | 1.00 | 25.96 | H |
| ATOM | 2190 | CG | ASN | H | 76 | 24.909 | 10.237 | −29.007 | 1.00 | 31.03 | H |
| ATOM | 2191 | OD1 | ASN | H | 76 | 24.399 | 10.514 | −30.094 | 1.00 | 36.46 | H |
| ATOM | 2192 | ND2 | ASN | H | 76 | 26.029 | 9.526 | −28.900 | 1.00 | 37.11 | H |
| ATOM | 2193 | C | ASN | H | 76 | 22.128 | 10.738 | −26.435 | 1.00 | 23.09 | H |
| ATOM | 2194 | O | ASN | H | 76 | 22.762 | 10.318 | −25.469 | 1.00 | 21.27 | H |
| ATOM | 2195 | N | THR | H | 77 | 20.865 | 11.144 | −26.356 | 1.00 | 21.10 | H |
| ATOM | 2196 | CA | THR | H | 77 | 20.121 | 11.105 | −25.108 | 1.00 | 19.95 | H |
| ATOM | 2197 | CB | THR | H | 77 | 19.692 | 12.518 | −24.667 | 1.00 | 19.84 | H |
| ATOM | 2198 | OG1 | THR | H | 77 | 20.851 | 13.349 | −24.539 | 1.00 | 24.67 | H |
| ATOM | 2199 | CG2 | THR | H | 77 | 18.967 | 12.466 | −23.321 | 1.00 | 18.95 | H |
| ATOM | 2200 | C | THR | H | 77 | 18.873 | 10.245 | −25.261 | 1.00 | 19.67 | H |
| ATOM | 2201 | O | THR | H | 77 | 18.151 | 10.342 | −26.263 | 1.00 | 18.36 | H |
| ATOM | 2202 | N | ALA | H | 78 | 18.643 | 9.388 | −24.273 | 1.00 | 16.32 | H |
| ATOM | 2203 | CA | ALA | H | 78 | 17.473 | 8.525 | −24.253 | 1.00 | 16.61 | H |
| ATOM | 2204 | CB | ALA | H | 78 | 17.888 | 7.073 | −24.032 | 1.00 | 15.21 | H |
| ATOM | 2205 | C | ALA | H | 78 | 16.613 | 9.019 | −23.094 | 1.00 | 16.94 | H |
| ATOM | 2206 | O | ALA | H | 78 | 17.138 | 9.522 | −22.103 | 1.00 | 19.03 | H |
| ATOM | 2207 | N | TYR | H | 79 | 15.298 | 8.878 | −23.214 | 1.00 | 17.77 | H |
| ATOM | 2208 | CA | TYR | H | 79 | 14.390 | 9.350 | −22.172 | 1.00 | 16.54 | H |
| ATOM | 2209 | CB | TYR | H | 79 | 13.599 | 10.571 | −22.664 | 1.00 | 16.99 | H |
| ATOM | 2210 | CG | TYR | H | 79 | 14.429 | 11.770 | −23.036 | 1.00 | 17.49 | H |
| ATOM | 2211 | CD1 | TYR | H | 79 | 14.809 | 12.703 | −22.072 | 1.00 | 19.75 | H |
| ATOM | 2212 | CE1 | TYR | H | 79 | 15.580 | 13.810 | −22.406 | 1.00 | 18.31 | H |
| ATOM | 2213 | CD2 | TYR | H | 79 | 14.843 | 11.972 | −24.355 | 1.00 | 17.52 | H |
| ATOM | 2214 | CE2 | TYR | H | 79 | 15.618 | 13.080 | −24.702 | 1.00 | 18.80 | H |
| ATOM | 2215 | CZ | TYR | H | 79 | 15.983 | 13.991 | −23.725 | 1.00 | 18.67 | H |
| ATOM | 2216 | OH | TYR | H | 79 | 16.769 | 15.069 | −24.050 | 1.00 | 19.71 | H |
| ATOM | 2217 | C | TYR | H | 79 | 13.380 | 8.300 | −21.744 | 1.00 | 16.90 | H |
| ATOM | 2218 | O | TYR | H | 79 | 12.897 | 7.514 | −22.560 | 1.00 | 17.15 | H |
| ATOM | 2219 | N | LEU | H | 80 | 13.060 | 8.305 | −20.456 | 1.00 | 15.44 | H |
| ATOM | 2220 | CA | LEU | H | 80 | 12.050 | 7.406 | −19.914 | 1.00 | 15.40 | H |
| ATOM | 2221 | CB | LEU | H | 80 | 12.618 | 6.501 | −18.810 | 1.00 | 14.87 | H |
| ATOM | 2222 | CG | LEU | H | 80 | 11.526 | 5.799 | −17.985 | 1.00 | 15.37 | H |
| ATOM | 2223 | CD1 | LEU | H | 80 | 10.847 | 4.726 | −18.841 | 1.00 | 11.18 | H |
| ATOM | 2224 | CD2 | LEU | H | 80 | 12.128 | 5.185 | −16.709 | 1.00 | 14.33 | H |
| ATOM | 2225 | C | LEU | H | 80 | 10.971 | 8.285 | −19.304 | 1.00 | 14.24 | H |
| ATOM | 2226 | O | LEU | H | 80 | 11.267 | 9.160 | −18.494 | 1.00 | 12.88 | H |
| ATOM | 2227 | N | GLN | H | 81 | 9.727 | 8.077 | −19.716 | 1.00 | 14.26 | H |
| ATOM | 2228 | CA | GLN | H | 81 | 8.627 | 8.826 | −19.140 | 1.00 | 15.68 | H |
| ATOM | 2229 | CB | GLN | H | 81 | 7.883 | 9.652 | −20.197 | 1.00 | 22.30 | H |
| ATOM | 2230 | CG | GLN | H | 81 | 6.599 | 10.280 | −19.644 | 1.00 | 29.38 | H |
| ATOM | 2231 | CD | GLN | H | 81 | 5.861 | 11.147 | −20.651 | 1.00 | 35.77 | H |
| ATOM | 2232 | OE1 | GLN | H | 81 | 5.849 | 10.857 | −21.850 | 1.00 | 38.76 | H |
| ATOM | 2233 | NE2 | GLN | H | 81 | 5.220 | 12.207 | −20.163 | 1.00 | 36.96 | H |
| ATOM | 2234 | C | GLN | H | 81 | 7.668 | 7.832 | −18.500 | 1.00 | 14.17 | H |
| ATOM | 2235 | O | GLN | H | 81 | 7.259 | 6.863 | −19.134 | 1.00 | 11.10 | H |
| ATOM | 2236 | N | LEU | H | 82 | 7.320 | 8.073 | −17.238 | 1.00 | 15.53 | H |
| ATOM | 2237 | CA | LEU | H | 82 | 6.403 | 7.199 | −16.508 | 1.00 | 14.92 | H |
| ATOM | 2238 | CB | LEU | H | 82 | 7.124 | 6.557 | −15.322 | 1.00 | 12.70 | H |
| ATOM | 2239 | CG | LEU | H | 82 | 6.755 | 5.141 | −14.856 | 1.00 | 17.68 | H |
| ATOM | 2240 | CD1 | LEU | H | 82 | 7.087 | 5.023 | −13.376 | 1.00 | 10.38 | H |
| ATOM | 2241 | CD2 | LEU | H | 82 | 5.286 | 4.830 | −15.090 | 1.00 | 17.56 | H |
| ATOM | 2242 | C | LEU | H | 82 | 5.250 | 8.077 | −16.001 | 1.00 | 15.80 | H |
| ATOM | 2243 | O | LEU | H | 82 | 5.480 | 9.044 | −15.272 | 1.00 | 14.86 | H |
| ATOM | 2244 | N | ASN | H | 82A | 4.023 | 7.725 | −16.380 | 1.00 | 14.11 | H |
| ATOM | 2245 | CA | ASN | H | 82A | 2.827 | 8.489 | −16.005 | 1.00 | 16.21 | H |
| ATOM | 2246 | CB | ASN | H | 82A | 1.837 | 8.520 | −17.172 | 1.00 | 19.43 | H |
| ATOM | 2247 | CG | ASN | H | 82A | 2.458 | 9.020 | −18.451 | 1.00 | 26.73 | H |
| ATOM | 2248 | OD1 | ASN | H | 82A | 2.062 | 8.611 | −19.544 | 1.00 | 31.37 | H |
| ATOM | 2249 | ND2 | ASN | H | 82A | 3.426 | 9.918 | −18.330 | 1.00 | 29.73 | H |
| ATOM | 2250 | C | ASN | H | 82A | 2.069 | 7.946 | −14.806 | 1.00 | 15.71 | H |
| ATOM | 2251 | O | ASN | H | 82A | 2.368 | 6.865 | −14.290 | 1.00 | 13.94 | H |
| ATOM | 2252 | N | SER | H | 82B | 1.064 | 8.711 | −14.392 | 1.00 | 12.68 | H |
| ATOM | 2253 | CA | SER | H | 82B | 0.182 | 8.329 | −13.301 | 1.00 | 16.08 | H |
| ATOM | 2254 | CB | SER | H | 82B | −0.858 | 7.349 | −13.855 | 1.00 | 16.26 | H |
| ATOM | 2255 | OG | SER | H | 82B | −1.827 | 7.007 | −12.882 | 1.00 | 28.09 | H |
| ATOM | 2256 | C | SER | H | 82B | 0.945 | 7.709 | −12.127 | 1.00 | 15.97 | H |
| ATOM | 2257 | O | SER | H | 82B | 0.634 | 6.608 | −11.687 | 1.00 | 13.30 | H |
| ATOM | 2258 | N | LEU | H | 82C | 1.935 | 8.441 | −11.622 | 1.00 | 16.32 | H |
| ATOM | 2259 | CA | LEU | H | 82C | 2.777 | 7.974 | −10.524 | 1.00 | 15.98 | H |
| ATOM | 2260 | CB | LEU | H | 82C | 3.898 | 8.985 | −10.251 | 1.00 | 14.25 | H |
| ATOM | 2261 | CG | LEU | H | 82C | 5.277 | 8.887 | −10.922 | 1.00 | 18.81 | H |
| ATOM | 2262 | CD1 | LEU | H | 82C | 5.253 | 7.953 | −12.113 | 1.00 | 16.74 | H |
| ATOM | 2263 | CD2 | LEU | H | 82C | 5.731 | 10.294 | −11.323 | 1.00 | 11.42 | H |
| ATOM | 2264 | C | LEU | H | 82C | 2.062 | 7.680 | −9.219 | 1.00 | 15.95 | H |
| ATOM | 2265 | O | LEU | H | 82C | 1.167 | 8.412 | −8.796 | 1.00 | 14.22 | H |
| ATOM | 2266 | N | LYS | H | 83 | 2.488 | 6.590 | −8.594 | 1.00 | 16.60 | H |
| ATOM | 2267 | CA | LYS | H | 83 | 1.979 | 6.145 | −7.306 | 1.00 | 18.74 | H |
| ATOM | 2268 | CB | LYS | H | 83 | 1.232 | 4.819 | −7.437 | 1.00 | 19.46 | H |
| ATOM | 2269 | CG | LYS | H | 83 | −0.220 | 4.966 | −7.843 | 1.00 | 25.19 | H |
| ATOM | 2270 | CD | LYS | H | 83 | −0.905 | 3.611 | −7.866 | 1.00 | 29.65 | H |
| ATOM | 2271 | CE | LYS | H | 83 | −2.417 | 3.763 | −7.884 | 1.00 | 32.26 | H |
| ATOM | 2272 | NZ | LYS | H | 83 | −2.920 | 4.512 | −6.683 | 1.00 | 32.62 | H |
| ATOM | 2273 | C | LYS | H | 83 | 3.197 | 5.943 | −6.413 | 1.00 | 18.61 | H |
| ATOM | 2274 | O | LYS | H | 83 | 4.306 | 5.741 | −6.913 | 1.00 | 17.60 | H |
| ATOM | 2275 | N | PRO | H | 84 | 3.007 | 5.988 | −5.087 | 1.00 | 18.21 | H |
| ATOM | 2276 | CD | PRO | H | 84 | 1.733 | 6.206 | −4.375 | 1.00 | 18.59 | H |
| ATOM | 2277 | CA | PRO | H | 84 | 4.113 | 5.805 | −4.141 | 1.00 | 20.09 | H |
| ATOM | 2278 | CB | PRO | H | 84 | 3.404 | 5.710 | −2.791 | 1.00 | 20.25 | H |
| ATOM | 2279 | CG | PRO | H | 84 | 2.201 | 6.600 | −2.989 | 1.00 | 19.66 | H |
| ATOM | 2280 | C | PRO | H | 84 | 4.957 | 4.566 | −4.447 | 1.00 | 19.54 | H |
| ATOM | 2281 | O | PRO | H | 84 | 6.164 | 4.562 | −4.216 | 1.00 | 21.23 | H |
| ATOM | 2282 | N | GLU | H | 85 | 4.326 | 3.524 | −4.978 | 1.00 | 19.06 | H |
| ATOM | 2283 | CA | GLU | H | 85 | 5.044 | 2.298 | −5.304 | 1.00 | 18.72 | H |
| ATOM | 2284 | CB | GLU | H | 85 | 4.071 | 1.159 | −5.639 | 1.00 | 21.75 | H |
| ATOM | 2285 | CG | GLU | H | 85 | 2.930 | 0.999 | −4.657 | 1.00 | 28.99 | H |
| ATOM | 2286 | CD | GLU | H | 85 | 1.777 | 1.935 | −4.968 | 1.00 | 30.56 | H |
| ATOM | 2287 | OE1 | GLU | H | 85 | 1.058 | 1.678 | −5.958 | 1.00 | 34.47 | H |
| ATOM | 2288 | OE2 | GLU | H | 85 | 1.596 | 2.928 | −4.232 | 1.00 | 31.63 | H |
| ATOM | 2289 | C | GLU | H | 85 | 6.007 | 2.481 | −6.475 | 1.00 | 17.32 | H |
| ATOM | 2290 | O | GLU | H | 85 | 6.770 | 1.566 | −6.799 | 1.00 | 16.06 | H |
| ATOM | 2291 | N | ASP | H | 86 | 5.957 | 3.639 | −7.126 | 1.00 | 12.60 | H |
| ATOM | 2292 | CA | ASP | H | 86 | 6.861 | 3.908 | −8.246 | 1.00 | 14.20 | H |
| ATOM | 2293 | CB | ASP | H | 86 | 6.203 | 4.831 | −9.280 | 1.00 | 13.35 | H |
| ATOM | 2294 | CG | ASP | H | 86 | 5.049 | 4.158 | −10.017 | 1.00 | 16.05 | H |
| ATOM | 2295 | OD1 | ASP | H | 86 | 5.273 | 3.086 | −10.614 | 1.00 | 16.03 | H |
| ATOM | 2296 | OD2 | ASP | H | 86 | 3.918 | 4.701 | −10.008 | 1.00 | 16.06 | H |
| ATOM | 2297 | C | ASP | H | 86 | 8.146 | 4.541 | −7.711 | 1.00 | 14.26 | H |
| ATOM | 2298 | O | ASP | H | 86 | 9.091 | 4.790 | −8.460 | 1.00 | 17.20 | H |
| ATOM | 2299 | N | THR | H | 87 | 8.168 | 4.810 | −6.410 | 1.00 | 13.02 | H |
| ATOM | 2300 | CA | THR | H | 87 | 9.351 | 5.375 | −5.777 | 1.00 | 12.35 | H |
| ATOM | 2301 | CB | THR | H | 87 | 9.127 | 5.586 | −4.281 | 1.00 | 12.53 | H |
| ATOM | 2302 | OG1 | THR | H | 87 | 8.171 | 6.631 | −4.093 | 1.00 | 14.67 | H |
| ATOM | 2303 | CG2 | THR | H | 87 | 10.439 | 5.964 | −3.586 | 1.00 | 16.61 | H |
| ATOM | 2304 | C | THR | H | 87 | 10.448 | 4.337 | −5.972 | 1.00 | 12.95 | H |
| ATOM | 2305 | O | THR | H | 87 | 10.308 | 3.188 | −5.540 | 1.00 | 12.66 | H |
| ATOM | 2306 | N | ALA | H | 88 | 11.532 | 4.737 | −6.625 | 1.00 | 13.68 | H |
| ATOM | 2307 | CA | ALA | H | 88 | 12.621 | 3.812 | −6.887 | 1.00 | 13.17 | H |
| ATOM | 2308 | CB | ALA | H | 88 | 12.109 | 2.652 | −7.738 | 1.00 | 13.61 | H |
| ATOM | 2309 | C | ALA | H | 88 | 13.765 | 4.493 | −7.613 | 1.00 | 15.16 | H |
| ATOM | 2310 | O | ALA | H | 88 | 13.657 | 5.659 | −8.011 | 1.00 | 11.84 | H |
| ATOM | 2311 | N | VAL | H | 89 | 14.868 | 3.759 | −7.752 | 1.00 | 14.10 | H |
| ATOM | 2312 | CA | VAL | H | 89 | 16.016 | 4.243 | −8.494 | 1.00 | 13.97 | H |
| ATOM | 2313 | CB | VAL | H | 89 | 17.353 | 3.654 | −7.974 | 1.00 | 16.92 | H |
| ATOM | 2314 | CG1 | VAL | H | 89 | 18.485 | 4.041 | −8.926 | 1.00 | 12.03 | H |
| ATOM | 2315 | CG2 | VAL | H | 89 | 17.654 | 4.168 | −6.567 | 1.00 | 14.95 | H |
| ATOM | 2316 | C | VAL | H | 89 | 15.785 | 3.721 | −9.910 | 1.00 | 14.69 | H |
| ATOM | 2317 | O | VAL | H | 89 | 15.467 | 2.540 | −10.096 | 1.00 | 16.19 | H |
| ATOM | 2318 | N | TYR | H | 90 | 15.913 | 4.593 | −10.904 | 1.00 | 13.30 | H |
| ATOM | 2319 | CA | TYR | H | 90 | 15.727 | 4.184 | −12.291 | 1.00 | 14.31 | H |
| ATOM | 2320 | CB | TYR | H | 90 | 14.714 | 5.103 | −13.003 | 1.00 | 14.28 | H |
| ATOM | 2321 | CG | TYR | H | 90 | 13.291 | 4.953 | −12.496 | 1.00 | 12.16 | H |
| ATOM | 2322 | CD1 | TYR | H | 90 | 12.922 | 5.442 | −11.242 | 1.00 | 11.31 | H |
| ATOM | 2323 | CE1 | TYR | H | 90 | 11.652 | 5.220 | −10.727 | 1.00 | 11.39 | H |
| ATOM | 2324 | CD2 | TYR | H | 90 | 12.337 | 4.241 | −13.231 | 1.00 | 12.99 | H |
| ATOM | 2325 | CE2 | TYR | H | 90 | 11.058 | 4.008 | −12.719 | 1.00 | 11.06 | H |
| ATOM | 2326 | CZ | TYR | H | 90 | 10.729 | 4.496 | −11.468 | 1.00 | 11.40 | H |
| ATOM | 2327 | OH | TYR | H | 90 | 9.496 | 4.219 | −10.931 | 1.00 | 15.00 | H |
| ATOM | 2328 | C | TYR | H | 90 | 17.070 | 4.242 | −13.004 | 1.00 | 15.02 | H |
| ATOM | 2329 | O | TYR | H | 90 | 17.749 | 5.271 | −12.965 | 1.00 | 15.56 | H |
| ATOM | 2330 | N | TYR | H | 91 | 17.460 | 3.127 | −13.622 | 1.00 | 14.85 | H |
| ATOM | 2331 | CA | TYR | H | 91 | 18.718 | 3.043 | −14.361 | 1.00 | 12.74 | H |
| ATOM | 2332 | CB | TYR | H | 91 | 19.570 | 1.828 | −13.949 | 1.00 | 13.34 | H |
| ATOM | 2333 | CG | TYR | H | 91 | 19.977 | 1.707 | −12.500 | 1.00 | 14.12 | H |
| ATOM | 2334 | CD1 | TYR | H | 91 | 19.205 | 0.968 | −11.602 | 1.00 | 13.71 | H |
| ATOM | 2335 | CE1 | TYR | H | 91 | 19.590 | 0.822 | −10.279 | 1.00 | 14.75 | H |
| ATOM | 2336 | CD2 | TYR | H | 91 | 21.150 | 2.299 | −12.033 | 1.00 | 10.40 | H |
| ATOM | 2337 | CE2 | TYR | H | 91 | 21.544 | 2.159 | −10.707 | 1.00 | 12.54 | H |
| ATOM | 2338 | CZ | TYR | H | 91 | 20.761 | 1.422 | −9.836 | 1.00 | 12.79 | H |
| ATOM | 2339 | OH | TYR | H | 91 | 21.133 | 1.295 | −8.517 | 1.00 | 15.61 | H |
| ATOM | 2340 | C | TYR | H | 91 | 18.500 | 2.854 | −15.850 | 1.00 | 13.74 | H |
| ATOM | 2341 | O | TYR | H | 91 | 17.575 | 2.151 | −16.270 | 1.00 | 11.06 | H |
| ATOM | 2342 | N | CYS | H | 92 | 19.353 | 3.480 | −16.652 | 1.00 | 12.91 | H |
| ATOM | 2343 | CA | CYS | H | 92 | 19.309 | 3.237 | −18.081 | 1.00 | 16.65 | H |
| ATOM | 2344 | C | CYS | H | 92 | 20.483 | 2.270 | −18.222 | 1.00 | 16.22 | H |
| ATOM | 2345 | O | CYS | H | 92 | 21.407 | 2.284 | −17.409 | 1.00 | 15.01 | H |
| ATOM | 2346 | CB | CYS | H | 92 | 19.564 | 4.501 | −18.921 | 1.00 | 19.88 | H |
| ATOM | 2347 | SG | CYS | H | 92 | 20.992 | 5.544 | −18.482 | 1.00 | 25.81 | H |
| ATOM | 2348 | N | ALA | H | 93 | 20.444 | 1.422 | −19.232 | 1.00 | 16.85 | H |
| ATOM | 2349 | CA | ALA | H | 93 | 21.513 | 0.468 | −19.428 | 1.00 | 20.24 | H |
| ATOM | 2350 | CB | ALA | H | 93 | 21.250 | −0.784 | −18.600 | 1.00 | 21.79 | H |
| ATOM | 2351 | C | ALA | H | 93 | 21.625 | 0.105 | −20.887 | 1.00 | 21.02 | H |
| ATOM | 2352 | O | ALA | H | 93 | 20.630 | −0.239 | −21.529 | 1.00 | 21.48 | H |
| ATOM | 2353 | N | ARG | H | 94 | 22.841 | 0.186 | −21.413 | 1.00 | 20.12 | H |
| ATOM | 2354 | CA | ARG | H | 94 | 23.054 | −0.165 | −22.798 | 1.00 | 19.46 | H |
| ATOM | 2355 | CB | ARG | H | 94 | 23.936 | 0.864 | −23.512 | 1.00 | 20.68 | H |
| ATOM | 2356 | CG | ARG | H | 94 | 24.103 | 0.553 | −24.999 | 1.00 | 24.06 | H |
| ATOM | 2357 | CD | ARG | H | 94 | 25.274 | 1.288 | −25.624 | 1.00 | 23.68 | H |
| ATOM | 2358 | NE | ARG | H | 94 | 24.992 | 2.700 | −25.822 | 1.00 | 27.40 | H |
| ATOM | 2359 | CZ | ARG | H | 94 | 25.856 | 3.568 | −26.336 | 1.00 | 26.78 | H |
| ATOM | 2360 | NH1 | ARG | H | 94 | 27.065 | 3.168 | −26.706 | 1.00 | 27.62 | H |
| ATOM | 2361 | NH2 | ARG | H | 94 | 25.510 | 4.837 | −26.473 | 1.00 | 26.02 | H |
| ATOM | 2362 | C | ARG | H | 94 | 23.695 | −1.542 | −22.933 | 1.00 | 18.01 | H |
| ATOM | 2363 | O | ARG | H | 94 | 24.522 | −1.951 | −22.125 | 1.00 | 17.99 | H |
| ATOM | 2364 | N | GLU | H | 95 | 23.261 | −2.237 | −23.972 | 1.00 | 17.90 | H |
| ATOM | 2365 | CA | GLU | H | 95 | 23.725 | −3.555 | −24.374 | 1.00 | 18.90 | H |
| ATOM | 2366 | CB | GLU | H | 95 | 23.142 | −3.797 | −25.756 | 1.00 | 22.87 | H |
| ATOM | 2367 | CG | GLU | H | 95 | 23.255 | −5.153 | −26.342 | 1.00 | 27.64 | H |
| ATOM | 2368 | CD | GLU | H | 95 | 22.615 | −5.185 | −27.715 | 1.00 | 27.44 | H |
| ATOM | 2369 | OE1 | GLU | H | 95 | 22.358 | −6.297 | −28.214 | 1.00 | 30.37 | H |
| ATOM | 2370 | OE2 | GLU | H | 95 | 22.377 | −4.090 | −28.296 | 1.00 | 22.68 | H |
| ATOM | 2371 | C | GLU | H | 95 | 25.260 | −3.529 | −24.451 | 1.00 | 21.19 | H |
| ATOM | 2372 | O | GLU | H | 95 | 25.845 | −2.506 | −24.810 | 1.00 | 21.98 | H |
| ATOM | 2373 | N | GLY | H | 96 | 25.915 | −4.634 | −24.112 | 1.00 | 20.63 | H |
| ATOM | 2374 | CA | GLY | H | 96 | 27.364 | −4.666 | −24.206 | 1.00 | 22.13 | H |
| ATOM | 2375 | C | GLY | H | 96 | 27.772 | −5.136 | −25.593 | 1.00 | 23.52 | H |
| ATOM | 2376 | O | GLY | H | 96 | 26.909 | −5.379 | −26.438 | 1.00 | 23.31 | H |
| ATOM | 2377 | N | SER | H | 97 | 29.076 | −5.261 | −25.838 | 1.00 | 25.68 | H |
| ATOM | 2378 | CA | SER | H | 97 | 29.576 | −5.730 | −27.134 | 1.00 | 27.29 | H |
| ATOM | 2379 | CB | SER | H | 97 | 29.962 | −4.542 | −28.034 | 1.00 | 27.37 | H |
| ATOM | 2380 | OG | SER | H | 97 | 30.950 | −3.723 | −27.431 | 1.00 | 27.88 | H |
| ATOM | 2381 | C | SER | H | 97 | 30.776 | −6.663 | −26.950 | 1.00 | 29.40 | H |
| ATOM | 2382 | O | SER | H | 97 | 31.430 | −6.650 | −25.913 | 1.00 | 29.06 | H |
| ATOM | 2383 | N | SER | H | 98 | 31.062 | −7.478 | −27.960 | 1.00 | 30.55 | H |
| ATOM | 2384 | CA | SER | H | 98 | 32.180 | −8.410 | −27.880 | 1.00 | 33.21 | H |
| ATOM | 2385 | CB | SER | H | 98 | 31.853 | −9.678 | −28.659 | 1.00 | 32.11 | H |
| ATOM | 2386 | OG | SER | H | 98 | 31.640 | −9.376 | −30.026 | 0.60 | 29.78 | H |
| ATOM | 2387 | C | SER | H | 98 | 33.464 | −7.805 | −28.435 | 1.00 | 35.19 | H |
| ATOM | 2388 | O | SER | H | 98 | 33.428 | −6.834 | −29.188 | 1.00 | 36.13 | H |
| ATOM | 2389 | N | GLY | H | 99 | 34.597 | −8.384 | −28.050 | 1.00 | 36.79 | H |
| ATOM | 2390 | CA | GLY | H | 99 | 35.883 | −7.916 | −28.542 | 1.00 | 39.03 | H |
| ATOM | 2391 | C | GLY | H | 99 | 36.381 | −6.579 | −28.029 | 1.00 | 40.02 | H |
| ATOM | 2392 | O | GLY | H | 99 | 35.738 | −5.930 | −27.205 | 1.00 | 39.67 | H |
| ATOM | 2393 | N | GLU | H | 100 | 37.548 | −6.174 | −28.525 | 1.00 | 41.33 | H |
| ATOM | 2394 | CA | GLU | H | 100 | 38.159 | −4.909 | −28.138 | 1.00 | 42.12 | H |
| ATOM | 2395 | CB | GLU | H | 100 | 39.593 | −4.830 | −28.673 | 1.00 | 43.63 | H |
| ATOM | 2396 | CG | GLU | H | 100 | 40.669 | −5.010 | −27.608 | 1.00 | 45.07 | H |
| ATOM | 2397 | CD | GLU | H | 100 | 41.602 | −6.168 | −27.900 | 1.00 | 46.46 | H |
| ATOM | 2398 | OE1 | GLU | H | 100 | 42.083 | −6.267 | −29.050 | 1.00 | 47.33 | H |
| ATOM | 2399 | OE2 | GLU | H | 100 | 41.860 | −6.971 | −26.976 | 1.00 | 45.78 | H |
| ATOM | 2400 | C | GLU | H | 100 | 37.347 | −3.727 | −28.656 | 1.00 | 42.27 | H |
| ATOM | 2401 | O | GLU | H | 100 | 36.628 | −3.841 | −29.652 | 1.00 | 42.00 | H |
| ATOM | 2402 | N | GLY | H | 100A | 37.469 | −2.592 | −27.976 | 1.00 | 41.38 | H |
| ATOM | 2403 | CA | GLY | H | 100A | 36.732 | −1.406 | −28.376 | 1.00 | 41.12 | H |
| ATOM | 2404 | C | GLY | H | 100A | 35.297 | −1.479 | −27.895 | 1.00 | 40.27 | H |
| ATOM | 2405 | O | GLY | H | 100A | 34.965 | −2.320 | −27.060 | 1.00 | 40.42 | H |
| ATOM | 2406 | N | TRP | H | 100B | 34.443 | −0.608 | −28.423 | 1.00 | 39.70 | H |
| ATOM | 2407 | CA | TRP | H | 100B | 33.038 | −0.591 | −28.030 | 1.00 | 39.10 | H |
| ATOM | 2408 | CB | TRP | H | 100B | 32.778 | 0.598 | −27.105 | 1.00 | 40.69 | H |
| ATOM | 2409 | CG | TRP | H | 100B | 33.605 | 0.544 | −25.866 | 1.00 | 41.43 | H |
| ATOM | 2410 | CD2 | TRP | H | 100B | 34.945 | 1.023 | −25.716 | 1.00 | 42.81 | H |
| ATOM | 2411 | CE2 | TRP | H | 100B | 35.359 | 0.704 | −24.403 | 1.00 | 42.90 | H |
| ATOM | 2412 | CE3 | TRP | H | 100B | 35.840 | 1.690 | −26.565 | 1.00 | 42.24 | H |
| ATOM | 2413 | CD1 | TRP | H | 100B | 33.266 | −0.028 | −24.674 | 1.00 | 42.27 | H |
| ATOM | 2414 | NE1 | TRP | H | 100B | 34.314 | 0.063 | −23.788 | 1.00 | 42.44 | H |
| ATOM | 2415 | CZ2 | TRP | H | 100B | 36.630 | 1.029 | −23.917 | 1.00 | 43.02 | H |
| ATOM | 2416 | CZ3 | TRP | H | 100B | 37.105 | 2.014 | −26.082 | 1.00 | 42.41 | H |
| ATOM | 2417 | CH2 | TRP | H | 100B | 37.487 | 1.682 | −24.769 | 1.00 | 43.01 | H |
| ATOM | 2418 | C | TRP | H | 100B | 32.105 | −0.537 | −29.235 | 1.00 | 38.08 | H |
| ATOM | 2419 | O | TRP | H | 100B | 31.022 | 0.049 | −29.171 | 1.00 | 37.44 | H |
| ATOM | 2420 | N | SER | H | 100C | 32.527 | −1.163 | −30.330 | 1.00 | 36.57 | H |
| ATOM | 2421 | CA | SER | H | 100C | 31.736 | −1.193 | −31.550 | 1.00 | 34.79 | H |
| ATOM | 2422 | CB | SER | H | 100C | 32.344 | −0.249 | −32.589 | 1.00 | 35.83 | H |
| ATOM | 2423 | OG | SER | H | 100C | 32.337 | 1.092 | −32.125 | 1.00 | 37.54 | H |
| ATOM | 2424 | C | SER | H | 100C | 31.656 | −2.607 | −32.113 | 1.00 | 33.66 | H |
| ATOM | 2425 | O | SER | H | 100C | 31.399 | −2.802 | −33.302 | 1.00 | 31.54 | H |
| ATOM | 2426 | N | GLY | H | 100D | 31.879 | −3.593 | −31.249 | 1.00 | 34.02 | H |
| ATOM | 2427 | CA | GLY | H | 100D | 31.820 | −4.980 | −31.675 | 1.00 | 33.38 | H |
| ATOM | 2428 | C | GLY | H | 100D | 30.387 | −5.479 | −31.750 | 1.00 | 33.47 | H |
| ATOM | 2429 | O | GLY | H | 100D | 29.449 | −4.679 | −31.745 | 1.00 | 31.00 | H |
| ATOM | 2430 | N | SER | H | 100E | 30.219 | −6.797 | −31.821 | 1.00 | 32.39 | H |
| ATOM | 2431 | CA | SER | H | 100E | 28.896 | −7.409 | −31.890 | 1.00 | 32.77 | H |
| ATOM | 2432 | CB | SER | H | 100E | 29.023 | −8.897 | −32.215 | 1.00 | 33.96 | H |
| ATOM | 2433 | OG | SER | H | 100E | 29.781 | −9.096 | −33.393 | 1.00 | 41.75 | H |
| ATOM | 2434 | C | SER | H | 100E | 28.161 | −7.251 | −30.560 | 1.00 | 31.30 | H |
| ATOM | 2435 | O | SER | H | 100E | 28.706 | −7.564 | −29.501 | 1.00 | 32.29 | H |
| ATOM | 2436 | N | PRO | H | 100F | 26.909 | −6.774 | −30.600 | 1.00 | 28.81 | H |
| ATOM | 2437 | CD | PRO | H | 100F | 26.129 | −6.437 | −31.807 | 1.00 | 28.48 | H |
| ATOM | 2438 | CA | PRO | H | 100F | 26.105 | −6.580 | −29.387 | 1.00 | 27.96 | H |
| ATOM | 2439 | CB | PRO | H | 100F | 24.899 | −5.798 | −29.902 | 1.00 | 27.33 | H |
| ATOM | 2440 | CG | PRO | H | 100F | 24.706 | −6.386 | −31.273 | 1.00 | 28.38 | H |
| ATOM | 2441 | C | PRO | H | 100F | 25.699 | −7.915 | −28.773 | 1.00 | 25.97 | H |
| ATOM | 2442 | O | PRO | H | 100F | 25.444 | −8.870 | −29.496 | 1.00 | 25.04 | H |
| ATOM | 2443 | N | ASP | H | 100G | 25.636 | −7.993 | −27.446 | 1.00 | 25.78 | H |
| ATOM | 2444 | CA | ASP | H | 100G | 25.240 | −9.250 | −26.828 | 1.00 | 25.70 | H |
| ATOM | 2445 | CB | ASP | H | 100G | 26.397 | −9.851 | −26.015 | 1.00 | 28.92 | H |
| ATOM | 2446 | CG | ASP | H | 100G | 27.170 | −8.820 | −25.230 | 1.00 | 33.23 | H |
| ATOM | 2447 | OD1 | ASP | H | 100G | 26.664 | −8.335 | −24.190 | 1.00 | 28.94 | H |
| ATOM | 2448 | OD2 | ASP | H | 100G | 28.301 | −8.502 | −25.664 | 1.00 | 37.37 | H |
| ATOM | 2449 | C | ASP | H | 100G | 23.963 | −9.225 | −25.997 | 1.00 | 22.11 | H |
| ATOM | 2450 | O | ASP | H | 100G | 23.724 | −10.127 | −25.202 | 1.00 | 22.36 | H |
| ATOM | 2451 | N | GLY | H | 100H | 23.140 | −8.199 | −26.192 | 1.00 | 20.73 | H |
| ATOM | 2452 | CA | GLY | H | 100H | 21.874 | −8.121 | −25.478 | 1.00 | 18.22 | H |
| ATOM | 2453 | C | GLY | H | 100H | 21.965 | −7.776 | −24.011 | 1.00 | 17.56 | H |
| ATOM | 2454 | O | GLY | H | 100H | 21.336 | −6.816 | −23.566 | 1.00 | 16.82 | H |
| ATOM | 2455 | N | ALA | H | 100I | 22.727 | −8.567 | −23.257 | 1.00 | 16.58 | H |
| ATOM | 2456 | CA | ALA | H | 100I | 22.918 | −8.332 | −21.830 | 1.00 | 16.91 | H |
| ATOM | 2457 | CB | ALA | H | 100I | 23.939 | −9.327 | −21.273 | 1.00 | 16.23 | H |
| ATOM | 2458 | C | ALA | H | 100I | 23.386 | −6.887 | −21.591 | 1.00 | 17.55 | H |
| ATOM | 2459 | O | ALA | H | 100I | 24.029 | −6.285 | −22.448 | 1.00 | 16.54 | H |
| ATOM | 2460 | N | PHE | H | 100J | 23.074 | −6.352 | −20.415 | 1.00 | 17.68 | H |
| ATOM | 2461 | CA | PHE | H | 100J | 23.408 | −4.975 | −20.049 | 1.00 | 16.85 | H |
| ATOM | 2462 | CB | PHE | H | 100J | 22.348 | −4.466 | −19.067 | 1.00 | 14.90 | H |
| ATOM | 2463 | CG | PHE | H | 100J | 20.933 | −4.668 | −19.545 | 1.00 | 13.97 | H |
| ATOM | 2464 | CD1 | PHE | H | 100J | 19.891 | −4.812 | −18.631 | 1.00 | 14.38 | H |
| ATOM | 2465 | CD2 | PHE | H | 100J | 20.635 | −4.694 | −20.907 | 1.00 | 16.04 | H |
| ATOM | 2466 | CE1 | PHE | H | 100J | 18.576 | −4.978 | −19.062 | 1.00 | 10.28 | H |
| ATOM | 2467 | CE2 | PHE | H | 100J | 19.321 | −4.858 | −21.349 | 1.00 | 14.28 | H |
| ATOM | 2468 | CZ | PHE | H | 100J | 18.291 | −5.001 | −20.423 | 1.00 | 13.93 | H |
| ATOM | 2469 | C | PHE | H | 100J | 24.810 | −4.764 | −19.464 | 1.00 | 16.53 | H |
| ATOM | 2470 | O | PHE | H | 100J | 25.048 | −4.991 | −18.278 | 1.00 | 13.51 | H |
| ATOM | 2471 | N | ALA | H | 101 | 25.728 | −4.292 | −20.299 | 1.00 | 17.73 | H |
| ATOM | 2472 | CA | ALA | H | 101 | 27.101 | −4.063 | −19.869 | 1.00 | 17.15 | H |
| ATOM | 2473 | CB | ALA | H | 101 | 28.055 | −4.350 | −21.017 | 1.00 | 18.30 | H |
| ATOM | 2474 | C | ALA | H | 101 | 27.353 | −2.659 | −19.336 | 1.00 | 18.96 | H |
| ATOM | 2475 | O | ALA | H | 101 | 28.217 | −2.469 | −18.487 | 1.00 | 18.39 | H |
| ATOM | 2476 | N | PHE | H | 102 | 26.601 | −1.681 | −19.833 | 1.00 | 18.02 | H |
| ATOM | 2477 | CA | PHE | H | 102 | 26.783 | −0.298 | −19.408 | 1.00 | 19.10 | H |
| ATOM | 2478 | CB | PHE | H | 102 | 27.103 | 0.561 | −20.624 | 1.00 | 18.63 | H |
| ATOM | 2479 | CG | PHE | H | 102 | 28.166 | −0.028 | −21.479 | 1.00 | 21.67 | H |
| ATOM | 2480 | CD1 | PHE | H | 102 | 27.867 | −0.536 | −22.729 | 1.00 | 23.75 | H |
| ATOM | 2481 | CD2 | PHE | H | 102 | 29.453 | −0.176 | −20.988 | 1.00 | 23.95 | H |
| ATOM | 2482 | CE1 | PHE | H | 102 | 28.826 | −1.185 | −23.468 | 1.00 | 22.45 | H |
| ATOM | 2483 | CE2 | PHE | H | 102 | 30.417 | −0.825 | −21.723 | 1.00 | 24.44 | H |
| ATOM | 2484 | CZ | PHE | H | 102 | 30.104 | −1.333 | −22.965 | 1.00 | 25.72 | H |
| ATOM | 2485 | C | PHE | H | 102 | 25.580 | 0.256 | −18.667 | 1.00 | 18.27 | H |
| ATOM | 2486 | O | PHE | H | 102 | 24.452 | 0.233 | −19.162 | 1.00 | 21.05 | H |
| ATOM | 2487 | N | TRP | H | 103 | 25.841 | 0.758 | −17.470 | 1.00 | 17.21 | H |
| ATOM | 2488 | CA | TRP | H | 103 | 24.797 | 1.296 | −16.622 | 1.00 | 17.88 | H |
| ATOM | 2489 | CB | TRP | H | 103 | 24.684 | 0.459 | −15.346 | 1.00 | 17.49 | H |
| ATOM | 2490 | CG | TRP | H | 103 | 24.293 | −0.964 | −15.577 | 1.00 | 15.78 | H |
| ATOM | 2491 | CD2 | TRP | H | 103 | 23.030 | −1.568 | −15.264 | 1.00 | 14.44 | H |
| ATOM | 2492 | CE2 | TRP | H | 103 | 23.107 | −2.923 | −15.655 | 1.00 | 13.86 | H |
| ATOM | 2493 | CE3 | TRP | H | 103 | 21.840 | −1.094 | −14.695 | 1.00 | 15.84 | H |
| ATOM | 2494 | CD1 | TRP | H | 103 | 25.061 | −1.949 | −16.130 | 1.00 | 14.46 | H |
| ATOM | 2495 | NE1 | TRP | H | 103 | 24.355 | −3.126 | −16.180 | 1.00 | 15.61 | H |
| ATOM | 2496 | CZ2 | TRP | H | 103 | 22.045 | −3.812 | −15.492 | 1.00 | 13.40 | H |
| ATOM | 2497 | CZ3 | TRP | H | 103 | 20.778 | −1.982 | −14.535 | 1.00 | 15.15 | H |
| ATOM | 2498 | CH2 | TRP | H | 103 | 20.892 | −3.326 | −14.933 | 1.00 | 15.72 | H |
| ATOM | 2499 | C | TRP | H | 103 | 25.053 | 2.740 | −16.234 | 1.00 | 17.94 | H |
| ATOM | 2500 | O | TRP | H | 103 | 26.198 | 3.143 | −16.041 | 1.00 | 15.64 | H |
| ATOM | 2501 | N | GLY | H | 104 | 23.977 | 3.514 | −16.120 | 1.00 | 18.87 | H |
| ATOM | 2502 | CA | GLY | H | 104 | 24.107 | 4.895 | −15.700 | 1.00 | 16.28 | H |
| ATOM | 2503 | C | GLY | H | 104 | 24.231 | 4.854 | −14.186 | 1.00 | 19.28 | H |
| ATOM | 2504 | O | GLY | H | 104 | 24.210 | 3.766 | −13.604 | 1.00 | 19.05 | H |
| ATOM | 2505 | N | GLN | H | 105 | 24.349 | 6.006 | −13.534 | 1.00 | 18.79 | H |
| ATOM | 2506 | CA | GLN | H | 105 | 24.489 | 6.011 | −12.082 | 1.00 | 19.29 | H |
| ATOM | 2507 | CB | GLN | H | 105 | 25.143 | 7.317 | −11.601 | 1.00 | 19.37 | H |
| ATOM | 2508 | CG | GLN | H | 105 | 24.194 | 8.516 | −11.508 | 1.00 | 19.21 | H |
| ATOM | 2509 | CD | GLN | H | 105 | 24.034 | 9.260 | −12.822 | 1.00 | 20.31 | H |
| ATOM | 2510 | OE1 | GLN | H | 105 | 24.082 | 8.670 | −13.895 | 1.00 | 16.90 | H |
| ATOM | 2511 | NE2 | GLN | H | 105 | 23.828 | 10.568 | −12.736 | 1.00 | 21.95 | H |
| ATOM | 2512 | C | GLN | H | 105 | 23.143 | 5.826 | −11.378 | 1.00 | 19.23 | H |
| ATOM | 2513 | O | GLN | H | 105 | 23.091 | 5.655 | −10.162 | 1.00 | 19.62 | H |
| ATOM | 2514 | N | GLY | H | 106 | 22.059 | 5.860 | −12.144 | 1.00 | 19.41 | H |
| ATOM | 2515 | CA | GLY | H | 106 | 20.744 | 5.708 | −11.550 | 1.00 | 19.55 | H |
| ATOM | 2516 | C | GLY | H | 106 | 20.141 | 7.051 | −11.164 | 1.00 | 19.76 | H |
| ATOM | 2517 | O | GLY | H | 106 | 20.856 | 8.034 | −10.985 | 1.00 | 21.23 | H |
| ATOM | 2518 | N | THR | H | 107 | 18.820 | 7.095 | −11.040 | 1.00 | 18.89 | H |
| ATOM | 2519 | CA | THR | H | 107 | 18.115 | 8.321 | −10.684 | 1.00 | 16.55 | H |
| ATOM | 2520 | CB | THR | H | 107 | 17.428 | 8.950 | −11.912 | 1.00 | 16.05 | H |
| ATOM | 2521 | OG1 | THR | H | 107 | 18.413 | 9.320 | −12.882 | 1.00 | 18.39 | H |
| ATOM | 2522 | CG2 | THR | H | 107 | 16.631 | 10.183 | −11.505 | 1.00 | 21.50 | H |
| ATOM | 2523 | C | THR | H | 107 | 17.032 | 7.981 | −9.673 | 1.00 | 15.77 | H |
| ATOM | 2524 | O | THR | H | 107 | 16.118 | 7.220 | −9.975 | 1.00 | 14.87 | H |
| ATOM | 2525 | N | LEU | H | 108 | 17.127 | 8.539 | −8.475 | 1.00 | 14.07 | H |
| ATOM | 2526 | CA | LEU | H | 108 | 16.122 | 8.262 | −7.469 | 1.00 | 16.45 | H |
| ATOM | 2527 | CB | LEU | H | 108 | 16.668 | 8.537 | −6.067 | 1.00 | 15.24 | H |
| ATOM | 2528 | CG | LEU | H | 108 | 15.629 | 8.437 | −4.944 | 1.00 | 17.84 | H |
| ATOM | 2529 | CD1 | LEU | H | 108 | 15.066 | 7.025 | −4.881 | 1.00 | 14.61 | H |
| ATOM | 2530 | CD2 | LEU | H | 108 | 16.269 | 8.815 | −3.614 | 1.00 | 15.88 | H |
| ATOM | 2531 | C | LEU | H | 108 | 14.878 | 9.109 | −7.697 | 1.00 | 16.56 | H |
| ATOM | 2532 | O | LEU | H | 108 | 14.938 | 10.337 | −7.652 | 1.00 | 16.88 | H |
| ATOM | 2533 | N | VAL | H | 109 | 13.753 | 8.454 | −7.963 | 1.00 | 14.70 | H |
| ATOM | 2534 | CA | VAL | H | 109 | 12.506 | 9.177 | −8.146 | 1.00 | 14.20 | H |
| ATOM | 2535 | CB | VAL | H | 109 | 11.740 | 8.721 | −9.405 | 1.00 | 15.31 | H |
| ATOM | 2536 | CG1 | VAL | H | 109 | 10.409 | 9.471 | −9.499 | 1.00 | 16.04 | H |
| ATOM | 2537 | CG2 | VAL | H | 109 | 12.564 | 8.997 | −10.646 | 1.00 | 15.47 | H |
| ATOM | 2538 | C | VAL | H | 109 | 11.657 | 8.912 | −6.916 | 1.00 | 13.81 | H |
| ATOM | 2539 | O | VAL | H | 109 | 11.339 | 7.766 | −6.601 | 1.00 | 15.75 | H |
| ATOM | 2540 | N | THR | H | 110 | 11.318 | 9.976 | −6.202 | 1.00 | 14.00 | H |
| ATOM | 2541 | CA | THR | H | 110 | 10.507 | 9.860 | −5.002 | 1.00 | 13.45 | H |
| ATOM | 2542 | CB | THR | H | 110 | 11.137 | 10.636 | −3.828 | 1.00 | 13.02 | H |
| ATOM | 2543 | OG1 | THR | H | 110 | 12.442 | 10.116 | −3.558 | 1.00 | 16.22 | H |
| ATOM | 2544 | CG2 | THR | H | 110 | 10.278 | 10.494 | −2.579 | 1.00 | 10.95 | H |
| ATOM | 2545 | C | THR | H | 110 | 9.106 | 10.412 | −5.260 | 1.00 | 13.67 | H |
| ATOM | 2546 | O | THR | H | 110 | 8.941 | 11.601 | −5.518 | 1.00 | 14.04 | H |
| ATOM | 2547 | N | VAL | H | 111 | 8.108 | 9.539 | −5.200 | 1.00 | 14.48 | H |
| ATOM | 2548 | CA | VAL | H | 111 | 6.726 | 9.942 | −5.419 | 1.00 | 14.29 | H |
| ATOM | 2549 | CB | VAL | H | 111 | 5.928 | 8.828 | −6.112 | 1.00 | 13.21 | H |
| ATOM | 2550 | CG1 | VAL | H | 111 | 4.500 | 9.297 | −6.387 | 1.00 | 16.08 | H |
| ATOM | 2551 | CG2 | VAL | H | 111 | 6.618 | 8.443 | −7.410 | 1.00 | 14.84 | H |
| ATOM | 2552 | C | VAL | H | 111 | 6.121 | 10.230 | −4.058 | 1.00 | 15.37 | H |
| ATOM | 2553 | O | VAL | H | 111 | 5.946 | 9.328 | −3.238 | 1.00 | 17.22 | H |
| ATOM | 2554 | N | SER | H | 112 | 5.795 | 11.492 | −3.814 | 1.00 | 14.00 | H |
| ATOM | 2555 | CA | SER | H | 112 | 5.260 | 11.878 | −2.515 | 1.00 | 13.79 | H |
| ATOM | 2556 | CB | SER | H | 112 | 6.415 | 11.910 | −1.505 | 1.00 | 14.08 | H |
| ATOM | 2557 | OG | SER | H | 112 | 6.039 | 12.530 | −0.293 | 1.00 | 12.76 | H |
| ATOM | 2558 | C | SER | H | 112 | 4.588 | 13.246 | −2.580 | 1.00 | 14.63 | H |
| ATOM | 2559 | O | SER | H | 112 | 4.840 | 14.026 | −3.503 | 1.00 | 14.19 | H |
| ATOM | 2560 | N | SER | H | 113 | 3.739 | 13.538 | −1.598 | 1.00 | 13.36 | H |
| ATOM | 2561 | CA | SER | H | 113 | 3.061 | 14.833 | −1.539 | 1.00 | 14.67 | H |
| ATOM | 2562 | CB | SER | H | 113 | 1.585 | 14.654 | −1.149 | 1.00 | 14.39 | H |
| ATOM | 2563 | OG | SER | H | 113 | 0.937 | 13.717 | −1.990 | 0.60 | 17.81 | H |
| ATOM | 2564 | C | SER | H | 113 | 3.756 | 15.732 | −0.507 | 1.00 | 13.30 | H |
| ATOM | 2565 | O | SER | H | 113 | 3.352 | 16.873 | −0.288 | 1.00 | 13.53 | H |
| ATOM | 2566 | N | ALA | H | 114 | 4.802 | 15.209 | 0.123 | 1.00 | 13.46 | H |
| ATOM | 2567 | CA | ALA | H | 114 | 5.530 | 15.962 | 1.139 | 1.00 | 15.19 | H |
| ATOM | 2568 | CB | ALA | H | 114 | 6.494 | 15.036 | 1.896 | 1.00 | 16.11 | H |
| ATOM | 2569 | C | ALA | H | 114 | 6.300 | 17.150 | 0.576 | 1.00 | 14.48 | H |
| ATOM | 2570 | O | ALA | H | 114 | 6.705 | 17.161 | −0.583 | 1.00 | 12.51 | H |
| ATOM | 2571 | N | SER | H | 115 | 6.491 | 18.155 | 1.420 | 1.00 | 15.49 | H |
| ATOM | 2572 | CA | SER | H | 115 | 7.235 | 19.350 | 1.048 | 1.00 | 18.64 | H |
| ATOM | 2573 | CB | SER | H | 115 | 6.504 | 20.612 | 1.532 | 1.00 | 21.54 | H |
| ATOM | 2574 | OG | SER | H | 115 | 5.296 | 20.822 | 0.822 | 1.00 | 29.56 | H |
| ATOM | 2575 | C | SER | H | 115 | 8.597 | 19.266 | 1.733 | 1.00 | 17.02 | H |
| ATOM | 2576 | O | SER | H | 115 | 8.737 | 18.605 | 2.764 | 1.00 | 19.25 | H |
| ATOM | 2577 | N | THR | H | 116 | 9.589 | 19.934 | 1.159 | 1.00 | 16.43 | H |
| ATOM | 2578 | CA | THR | H | 116 | 10.934 | 19.958 | 1.719 | 1.00 | 16.81 | H |
| ATOM | 2579 | CB | THR | H | 116 | 11.814 | 20.927 | 0.930 | 1.00 | 18.18 | H |
| ATOM | 2580 | OG1 | THR | H | 116 | 11.898 | 20.478 | −0.423 | 1.00 | 18.24 | H |
| ATOM | 2581 | CG2 | THR | H | 116 | 13.204 | 21.011 | 1.531 | 1.00 | 16.11 | H |
| ATOM | 2582 | C | THR | H | 116 | 10.901 | 20.398 | 3.184 | 1.00 | 16.28 | H |
| ATOM | 2583 | O | THR | H | 116 | 10.196 | 21.340 | 3.550 | 1.00 | 16.72 | H |
| ATOM | 2584 | N | LYS | H | 117 | 11.662 | 19.710 | 4.025 | 1.00 | 15.01 | H |
| ATOM | 2585 | CA | LYS | H | 117 | 11.694 | 20.044 | 5.441 | 1.00 | 14.63 | H |
| ATOM | 2586 | CB | LYS | H | 117 | 10.505 | 19.391 | 6.159 | 1.00 | 15.43 | H |
| ATOM | 2587 | CG | LYS | H | 117 | 10.356 | 19.788 | 7.622 | 1.00 | 14.95 | H |
| ATOM | 2588 | CD | LYS | H | 117 | 9.292 | 18.950 | 8.320 | 1.00 | 18.78 | H |
| ATOM | 2589 | CE | LYS | H | 117 | 9.256 | 19.217 | 9.831 | 1.00 | 22.36 | H |
| ATOM | 2590 | NZ | LYS | H | 117 | 10.577 | 18.912 | 10.494 | 1.00 | 28.51 | H |
| ATOM | 2591 | C | LYS | H | 117 | 12.997 | 19.590 | 6.089 | 1.00 | 15.60 | H |
| ATOM | 2592 | O | LYS | H | 117 | 13.470 | 18.473 | 5.858 | 1.00 | 15.20 | H |
| ATOM | 2593 | N | GLY | H | 118 | 13.571 | 20.467 | 6.905 | 1.00 | 15.94 | H |
| ATOM | 2594 | CA | GLY | H | 118 | 14.808 | 20.144 | 7.588 | 1.00 | 15.03 | H |
| ATOM | 2595 | C | GLY | H | 118 | 14.493 | 19.331 | 8.822 | 1.00 | 15.25 | H |
| ATOM | 2596 | O | GLY | H | 118 | 13.448 | 19.513 | 9.440 | 1.00 | 15.11 | H |
| ATOM | 2597 | N | PRO | H | 119 | 15.389 | 18.422 | 9.215 | 1.00 | 14.44 | H |
| ATOM | 2598 | CD | PRO | H | 119 | 16.669 | 18.090 | 8.558 | 1.00 | 12.53 | H |
| ATOM | 2599 | CA | PRO | H | 119 | 15.168 | 17.585 | 10.394 | 1.00 | 14.19 | H |
| ATOM | 2600 | CB | PRO | H | 119 | 16.069 | 16.396 | 10.121 | 1.00 | 13.47 | H |
| ATOM | 2601 | CG | PRO | H | 119 | 17.289 | 17.082 | 9.519 | 1.00 | 12.69 | H |
| ATOM | 2602 | C | PRO | H | 119 | 15.574 | 18.261 | 11.694 | 1.00 | 14.49 | H |
| ATOM | 2603 | O | PRO | H | 119 | 16.336 | 19.217 | 11.685 | 1.00 | 15.67 | H |
| ATOM | 2604 | N | SER | H | 120 | 15.055 | 17.761 | 12.807 | 1.00 | 13.98 | H |
| ATOM | 2605 | CA | SER | H | 120 | 15.480 | 18.247 | 14.112 | 1.00 | 17.16 | H |
| ATOM | 2606 | CB | SER | H | 120 | 14.355 | 18.170 | 15.147 | 1.00 | 16.79 | H |
| ATOM | 2607 | OG | SER | H | 120 | 13.209 | 18.886 | 14.722 | 1.00 | 25.25 | H |
| ATOM | 2608 | C | SER | H | 120 | 16.536 | 17.189 | 14.437 | 1.00 | 18.05 | H |
| ATOM | 2609 | O | SER | H | 120 | 16.416 | 16.042 | 13.987 | 1.00 | 17.07 | H |
| ATOM | 2610 | N | VAL | H | 121 | 17.578 | 17.556 | 15.175 | 1.00 | 17.40 | H |
| ATOM | 2611 | CA | VAL | H | 121 | 18.598 | 16.581 | 15.524 | 1.00 | 14.03 | H |
| ATOM | 2612 | CB | VAL | H | 121 | 20.000 | 17.001 | 15.005 | 1.00 | 14.58 | H |
| ATOM | 2613 | CG1 | VAL | H | 121 | 21.041 | 15.945 | 15.379 | 1.00 | 13.85 | H |
| ATOM | 2614 | CG2 | VAL | H | 121 | 19.965 | 17.180 | 13.492 | 1.00 | 8.66 | H |
| ATOM | 2615 | C | VAL | H | 121 | 18.611 | 16.447 | 17.039 | 1.00 | 16.59 | H |
| ATOM | 2616 | O | VAL | H | 121 | 18.967 | 17.382 | 17.755 | 1.00 | 17.80 | H |
| ATOM | 2617 | N | PHE | H | 122 | 18.206 | 15.279 | 17.525 | 1.00 | 15.70 | H |
| ATOM | 2618 | CA | PHE | H | 122 | 18.154 | 15.031 | 18.958 | 1.00 | 14.79 | H |
| ATOM | 2619 | CB | PHE | H | 122 | 16.780 | 14.480 | 19.349 | 1.00 | 14.90 | H |
| ATOM | 2620 | CG | PHE | H | 122 | 15.632 | 15.358 | 18.928 | 1.00 | 15.70 | H |
| ATOM | 2621 | CD1 | PHE | H | 122 | 15.563 | 16.686 | 19.346 | 1.00 | 15.66 | H |
| ATOM | 2622 | CD2 | PHE | H | 122 | 14.611 | 14.854 | 18.124 | 1.00 | 14.88 | H |
| ATOM | 2623 | CE1 | PHE | H | 122 | 14.487 | 17.500 | 18.967 | 1.00 | 19.60 | H |
| ATOM | 2624 | CE2 | PHE | H | 122 | 13.534 | 15.661 | 17.742 | 1.00 | 17.11 | H |
| ATOM | 2625 | CZ | PHE | H | 122 | 13.470 | 16.979 | 18.162 | 1.00 | 16.21 | H |
| ATOM | 2626 | C | PHE | H | 122 | 19.238 | 14.069 | 19.412 | 1.00 | 16.21 | H |
| ATOM | 2627 | O | PHE | H | 122 | 19.639 | 13.164 | 18.681 | 1.00 | 14.11 | H |
| ATOM | 2628 | N | PRO | H | 123 | 19.721 | 14.252 | 20.645 | 1.00 | 18.40 | H |
| ATOM | 2629 | CD | PRO | H | 123 | 19.405 | 15.353 | 21.575 | 1.00 | 17.09 | H |
| ATOM | 2630 | CA | PRO | H | 123 | 20.768 | 13.395 | 21.196 | 1.00 | 16.92 | H |
| ATOM | 2631 | CB | PRO | H | 123 | 21.360 | 14.259 | 22.297 | 1.00 | 16.43 | H |
| ATOM | 2632 | CG | PRO | H | 123 | 20.129 | 14.926 | 22.846 | 1.00 | 17.88 | H |
| ATOM | 2633 | C | PRO | H | 123 | 20.259 | 12.074 | 21.742 | 1.00 | 17.70 | H |
| ATOM | 2634 | O | PRO | H | 123 | 19.141 | 11.986 | 22.229 | 1.00 | 16.50 | H |
| ATOM | 2635 | N | LEU | H | 124 | 21.096 | 11.052 | 21.625 | 1.00 | 16.49 | H |
| ATOM | 2636 | CA | LEU | H | 124 | 20.826 | 9.726 | 22.164 | 1.00 | 17.77 | H |
| ATOM | 2637 | CB | LEU | H | 124 | 21.017 | 8.657 | 21.085 | 1.00 | 19.18 | H |
| ATOM | 2638 | CG | LEU | H | 124 | 19.806 | 8.091 | 20.329 | 1.00 | 19.90 | H |
| ATOM | 2639 | CD1 | LEU | H | 124 | 18.536 | 8.831 | 20.684 | 1.00 | 19.87 | H |
| ATOM | 2640 | CD2 | LEU | H | 124 | 20.073 | 8.153 | 18.845 | 1.00 | 18.45 | H |
| ATOM | 2641 | C | LEU | H | 124 | 21.940 | 9.646 | 23.207 | 1.00 | 17.76 | H |
| ATOM | 2642 | O | LEU | H | 124 | 23.017 | 9.126 | 22.936 | 1.00 | 15.95 | H |
| ATOM | 2643 | N | ALA | H | 125 | 21.680 | 10.209 | 24.383 | 1.00 | 18.36 | H |
| ATOM | 2644 | CA | ALA | H | 125 | 22.666 | 10.272 | 25.459 | 1.00 | 22.16 | H |
| ATOM | 2645 | CB | ALA | H | 125 | 22.096 | 11.071 | 26.638 | 1.00 | 18.54 | H |
| ATOM | 2646 | C | ALA | H | 125 | 23.204 | 8.937 | 25.960 | 1.00 | 22.76 | H |
| ATOM | 2647 | O | ALA | H | 125 | 22.478 | 7.954 | 26.055 | 1.00 | 23.82 | H |
| ATOM | 2648 | N | PRO | H | 126 | 24.504 | 8.889 | 26.282 | 1.00 | 26.11 | H |
| ATOM | 2649 | CD | PRO | H | 126 | 25.531 | 9.941 | 26.188 | 1.00 | 25.27 | H |
| ATOM | 2650 | CA | PRO | H | 126 | 25.074 | 7.632 | 26.781 | 1.00 | 28.93 | H |
| ATOM | 2651 | CB | PRO | H | 126 | 26.567 | 7.947 | 26.892 | 1.00 | 27.47 | H |
| ATOM | 2652 | CG | PRO | H | 126 | 26.592 | 9.430 | 27.131 | 1.00 | 26.40 | H |
| ATOM | 2653 | C | PRO | H | 126 | 24.431 | 7.300 | 28.128 | 1.00 | 32.90 | H |
| ATOM | 2654 | O | PRO | H | 126 | 24.299 | 8.169 | 28.989 | 1.00 | 33.12 | H |
| ATOM | 2655 | N | SER | H | 127 | 24.008 | 6.053 | 28.295 | 1.00 | 37.36 | H |
| ATOM | 2656 | CA | SER | H | 127 | 23.364 | 5.618 | 29.533 | 1.00 | 43.64 | H |
| ATOM | 2657 | CB | SER | H | 127 | 22.916 | 4.157 | 29.411 | 1.00 | 44.30 | H |
| ATOM | 2658 | OG | SER | H | 127 | 22.333 | 3.698 | 30.617 | 1.00 | 47.24 | H |
| ATOM | 2659 | C | SER | H | 127 | 24.273 | 5.766 | 30.749 | 1.00 | 46.48 | H |
| ATOM | 2660 | O | SER | H | 127 | 25.398 | 5.267 | 30.762 | 1.00 | 46.83 | H |
| ATOM | 2661 | N | SER | H | 128 | 23.772 | 6.453 | 31.771 | 1.00 | 50.29 | H |
| ATOM | 2662 | CA | SER | H | 128 | 24.529 | 6.664 | 32.998 | 1.00 | 54.76 | H |
| ATOM | 2663 | CB | SER | H | 128 | 23.868 | 7.753 | 33.847 | 1.00 | 54.72 | H |
| ATOM | 2664 | OG | SER | H | 128 | 22.547 | 7.385 | 34.207 | 1.00 | 55.54 | H |
| ATOM | 2665 | C | SER | H | 128 | 24.616 | 5.364 | 33.798 | 1.00 | 57.39 | H |
| ATOM | 2666 | O | SER | H | 128 | 25.484 | 5.211 | 34.657 | 1.00 | 58.15 | H |
| ATOM | 2667 | N | LYS | H | 129 | 23.707 | 4.436 | 33.508 | 1.00 | 60.71 | H |
| ATOM | 2668 | CA | LYS | H | 129 | 23.673 | 3.139 | 34.177 | 1.00 | 63.54 | H |
| ATOM | 2669 | CB | LYS | H | 129 | 22.233 | 2.621 | 34.264 | 1.00 | 64.23 | H |
| ATOM | 2670 | CG | LYS | H | 129 | 21.472 | 3.101 | 35.489 | 1.00 | 65.75 | H |
| ATOM | 2671 | CD | LYS | H | 129 | 22.145 | 2.626 | 36.777 | 1.00 | 66.61 | H |
| ATOM | 2672 | CE | LYS | H | 129 | 22.152 | 1.101 | 36.883 | 1.00 | 67.70 | H |
| ATOM | 2673 | NZ | LYS | H | 129 | 22.958 | 0.593 | 38.035 | 1.00 | 66.64 | H |
| ATOM | 2674 | C | LYS | H | 129 | 24.538 | 2.126 | 33.435 | 1.00 | 65.71 | H |
| ATOM | 2675 | O | LYS | H | 129 | 24.102 | 1.011 | 33.139 | 1.00 | 66.55 | H |
| ATOM | 2676 | N | SER | H | 130 | 25.769 | 2.528 | 33.140 | 1.00 | 67.40 | H |
| ATOM | 2677 | CA | SER | H | 130 | 26.726 | 1.689 | 32.426 | 1.00 | 68.20 | H |
| ATOM | 2678 | CB | SER | H | 130 | 26.478 | 1.769 | 30.921 | 1.00 | 67.87 | H |
| ATOM | 2679 | OG | SER | H | 130 | 26.719 | 3.083 | 30.440 | 1.00 | 66.73 | H |
| ATOM | 2680 | C | SER | H | 130 | 28.109 | 2.246 | 32.733 | 1.00 | 68.81 | H |
| ATOM | 2681 | O | SER | H | 130 | 29.132 | 1.668 | 32.361 | 1.00 | 69.06 | H |
| ATOM | 2682 | N | THR | H | 133 | 28.102 | 3.386 | 33.417 | 1.00 | 69.30 | H |
| ATOM | 2683 | CA | THR | H | 133 | 29.289 | 4.137 | 33.809 | 1.00 | 69.66 | H |
| ATOM | 2684 | CB | THR | H | 133 | 28.964 | 4.922 | 35.146 | 1.00 | 70.66 | H |
| ATOM | 2685 | OG1 | THR | H | 133 | 28.711 | 6.293 | 34.814 | 1.00 | 71.15 | H |
| ATOM | 2686 | CG2 | THR | H | 133 | 30.075 | 4.847 | 36.189 | 1.00 | 71.24 | H |
| ATOM | 2687 | C | THR | H | 133 | 30.642 | 3.399 | 33.827 | 1.00 | 68.61 | H |
| ATOM | 2688 | O | THR | H | 133 | 31.216 | 3.162 | 32.765 | 1.00 | 69.10 | H |
| ATOM | 2689 | N | SER | H | 134 | 31.154 | 3.027 | 34.994 | 1.00 | 66.54 | H |
| ATOM | 2690 | CA | SER | H | 134 | 32.451 | 2.367 | 35.073 | 1.00 | 63.79 | H |
| ATOM | 2691 | CB | SER | H | 134 | 32.811 | 2.101 | 36.534 | 1.00 | 64.81 | H |
| ATOM | 2692 | OG | SER | H | 134 | 33.030 | 3.320 | 37.220 | 1.00 | 65.31 | H |
| ATOM | 2693 | C | SER | H | 134 | 32.610 | 1.083 | 34.268 | 1.00 | 61.07 | H |
| ATOM | 2694 | O | SER | H | 134 | 31.774 | 0.180 | 34.324 | 1.00 | 60.75 | H |
| ATOM | 2695 | N | GLY | H | 135 | 33.707 | 1.022 | 33.520 | 1.00 | 57.68 | H |
| ATOM | 2696 | CA | GLY | H | 135 | 34.010 | −0.140 | 32.708 | 1.00 | 54.18 | H |
| ATOM | 2697 | C | GLY | H | 135 | 32.949 | −0.501 | 31.691 | 1.00 | 51.62 | H |
| ATOM | 2698 | O | GLY | H | 135 | 31.787 | −0.100 | 31.806 | 1.00 | 52.84 | H |
| ATOM | 2699 | N | GLY | H | 136 | 33.356 | −1.258 | 30.680 | 1.00 | 47.82 | H |
| ATOM | 2700 | CA | GLY | H | 136 | 32.418 | −1.682 | 29.663 | 1.00 | 43.20 | H |
| ATOM | 2701 | C | GLY | H | 136 | 32.309 | −0.779 | 28.453 | 1.00 | 39.61 | H |
| ATOM | 2702 | O | GLY | H | 136 | 33.116 | 0.125 | 28.243 | 1.00 | 38.63 | H |
| ATOM | 2703 | N | THR | H | 137 | 31.287 | −1.043 | 27.650 | 1.00 | 37.02 | H |
| ATOM | 2704 | CA | THR | H | 137 | 31.039 | −0.287 | 26.438 | 1.00 | 33.19 | H |
| ATOM | 2705 | CB | THR | H | 137 | 30.869 | −1.230 | 25.239 | 1.00 | 33.98 | H |
| ATOM | 2706 | OG1 | THR | H | 137 | 32.085 | −1.962 | 25.038 | 1.00 | 37.85 | H |
| ATOM | 2707 | CG2 | THR | H | 137 | 30.547 | −0.441 | 23.983 | 1.00 | 34.38 | H |
| ATOM | 2708 | C | THR | H | 137 | 29.781 | 0.545 | 26.587 | 1.00 | 28.74 | H |
| ATOM | 2709 | O | THR | H | 137 | 28.764 | 0.064 | 27.077 | 1.00 | 29.60 | H |
| ATOM | 2710 | N | ALA | H | 138 | 29.861 | 1.802 | 26.172 | 1.00 | 24.92 | H |
| ATOM | 2711 | CA | ALA | H | 138 | 28.719 | 2.699 | 26.239 | 1.00 | 21.06 | H |
| ATOM | 2712 | CB | ALA | H | 138 | 29.103 | 3.989 | 26.955 | 1.00 | 19.64 | H |
| ATOM | 2713 | C | ALA | H | 138 | 28.266 | 2.998 | 24.813 | 1.00 | 19.92 | H |
| ATOM | 2714 | O | ALA | H | 138 | 29.067 | 2.965 | 23.881 | 1.00 | 18.98 | H |
| ATOM | 2715 | N | ALA | H | 139 | 26.977 | 3.272 | 24.644 | 1.00 | 17.60 | H |
| ATOM | 2716 | CA | ALA | H | 139 | 26.440 | 3.584 | 23.328 | 1.00 | 15.58 | H |
| ATOM | 2717 | CB | ALA | H | 139 | 25.409 | 2.522 | 22.897 | 1.00 | 15.97 | H |
| ATOM | 2718 | C | ALA | H | 139 | 25.792 | 4.958 | 23.382 | 1.00 | 13.51 | H |
| ATOM | 2719 | O | ALA | H | 139 | 25.190 | 5.331 | 24.387 | 1.00 | 13.22 | H |
| ATOM | 2720 | N | LEU | H | 140 | 25.943 | 5.712 | 22.304 | 1.00 | 12.02 | H |
| ATOM | 2721 | CA | LEU | H | 140 | 25.358 | 7.040 | 22.199 | 1.00 | 13.12 | H |
| ATOM | 2722 | CB | LEU | H | 140 | 26.304 | 8.091 | 22.803 | 1.00 | 15.49 | H |
| ATOM | 2723 | CG | LEU | H | 140 | 27.695 | 8.260 | 22.184 | 1.00 | 15.97 | H |
| ATOM | 2724 | CD1 | LEU | H | 140 | 27.632 | 9.245 | 21.018 | 1.00 | 17.36 | H |
| ATOM | 2725 | CD2 | LEU | H | 140 | 28.660 | 8.776 | 23.239 | 1.00 | 19.65 | H |
| ATOM | 2726 | C | LEU | H | 140 | 25.136 | 7.290 | 20.713 | 1.00 | 12.42 | H |
| ATOM | 2727 | O | LEU | H | 140 | 25.645 | 6.550 | 19.878 | 1.00 | 12.05 | H |
| ATOM | 2728 | N | GLY | H | 141 | 24.367 | 8.321 | 20.386 | 1.00 | 13.33 | H |
| ATOM | 2729 | CA | GLY | H | 141 | 24.105 | 8.624 | 18.995 | 1.00 | 11.72 | H |
| ATOM | 2730 | C | GLY | H | 141 | 23.247 | 9.863 | 18.814 | 1.00 | 12.83 | H |
| ATOM | 2731 | O | GLY | H | 141 | 23.081 | 10.655 | 19.740 | 1.00 | 13.89 | H |
| ATOM | 2732 | N | CYS | H | 142 | 22.712 | 10.028 | 17.610 | 1.00 | 11.28 | H |
| ATOM | 2733 | CA | CYS | H | 142 | 21.856 | 11.158 | 17.279 | 1.00 | 12.60 | H |
| ATOM | 2734 | C | CYS | H | 142 | 20.624 | 10.681 | 16.519 | 1.00 | 12.75 | H |
| ATOM | 2735 | O | CYS | H | 142 | 20.703 | 9.761 | 15.706 | 1.00 | 15.35 | H |
| ATOM | 2736 | CB | CYS | H | 142 | 22.613 | 12.175 | 16.415 | 1.00 | 13.10 | H |
| ATOM | 2737 | SG | CYS | H | 142 | 23.789 | 13.245 | 17.312 | 1.00 | 22.67 | H |
| ATOM | 2738 | N | LEU | H | 143 | 19.488 | 11.310 | 16.790 | 1.00 | 12.56 | H |
| ATOM | 2739 | CA | LEU | H | 143 | 18.243 | 10.981 | 16.119 | 1.00 | 11.44 | H |
| ATOM | 2740 | CB | LEU | H | 143 | 17.121 | 10.789 | 17.139 | 1.00 | 11.98 | H |
| ATOM | 2741 | CG | LEU | H | 143 | 15.713 | 10.552 | 16.578 | 1.00 | 10.36 | H |
| ATOM | 2742 | CD1 | LEU | H | 143 | 15.706 | 9.305 | 15.697 | 1.00 | 10.52 | H |
| ATOM | 2743 | CD2 | LEU | H | 143 | 14.721 | 10.407 | 17.726 | 1.00 | 11.79 | H |
| ATOM | 2744 | C | LEU | H | 143 | 17.920 | 12.155 | 15.200 | 1.00 | 13.50 | H |
| ATOM | 2745 | O | LEU | H | 143 | 17.660 | 13.261 | 15.671 | 1.00 | 13.02 | H |
| ATOM | 2746 | N | VAL | H | 144 | 17.967 | 11.906 | 13.892 | 1.00 | 13.33 | H |
| ATOM | 2747 | CA | VAL | H | 144 | 17.697 | 12.922 | 12.868 | 1.00 | 14.30 | H |
| ATOM | 2748 | CB | VAL | H | 144 | 18.694 | 12.782 | 11.698 | 1.00 | 12.45 | H |
| ATOM | 2749 | CG1 | VAL | H | 144 | 18.510 | 13.925 | 10.712 | 1.00 | 13.61 | H |
| ATOM | 2750 | CG2 | VAL | H | 144 | 20.122 | 12.742 | 12.230 | 1.00 | 13.76 | H |
| ATOM | 2751 | C | VAL | H | 144 | 16.280 | 12.679 | 12.344 | 1.00 | 15.25 | H |
| ATOM | 2752 | O | VAL | H | 144 | 16.078 | 11.878 | 11.436 | 1.00 | 14.71 | H |
| ATOM | 2753 | N | LYS | H | 145 | 15.299 | 13.377 | 12.901 | 1.00 | 17.43 | H |
| ATOM | 2754 | CA | LYS | H | 145 | 13.931 | 13.127 | 12.490 | 1.00 | 19.01 | H |
| ATOM | 2755 | CB | LYS | H | 145 | 13.124 | 12.634 | 13.690 | 1.00 | 23.24 | H |
| ATOM | 2756 | CG | LYS | H | 145 | 12.661 | 13.711 | 14.628 | 1.00 | 27.84 | H |
| ATOM | 2757 | CD | LYS | H | 145 | 11.170 | 13.575 | 14.862 | 1.00 | 30.54 | H |
| ATOM | 2758 | CE | LYS | H | 145 | 10.835 | 12.220 | 15.436 | 1.00 | 31.67 | H |
| ATOM | 2759 | NZ | LYS | H | 145 | 9.409 | 11.871 | 15.191 | 1.00 | 30.62 | H |
| ATOM | 2760 | C | LYS | H | 145 | 13.143 | 14.217 | 11.787 | 1.00 | 17.76 | H |
| ATOM | 2761 | O | LYS | H | 145 | 13.411 | 15.411 | 11.937 | 1.00 | 17.23 | H |
| ATOM | 2762 | N | ASP | H | 146 | 12.154 | 13.753 | 11.025 | 1.00 | 15.26 | H |
| ATOM | 2763 | CA | ASP | H | 146 | 11.232 | 14.579 | 10.262 | 1.00 | 14.74 | H |
| ATOM | 2764 | CB | ASP | H | 146 | 10.383 | 15.436 | 11.207 | 1.00 | 15.34 | H |
| ATOM | 2765 | CG | ASP | H | 146 | 9.547 | 14.596 | 12.158 | 1.00 | 17.00 | H |
| ATOM | 2766 | OD1 | ASP | H | 146 | 9.385 | 13.384 | 11.897 | 1.00 | 15.00 | H |
| ATOM | 2767 | OD2 | ASP | H | 146 | 9.045 | 15.143 | 13.160 | 1.00 | 19.18 | H |
| ATOM | 2768 | C | ASP | H | 146 | 11.872 | 15.455 | 9.203 | 1.00 | 13.72 | H |
| ATOM | 2769 | O | ASP | H | 146 | 11.843 | 16.680 | 9.291 | 1.00 | 16.46 | H |
| ATOM | 2770 | N | TYR | H | 147 | 12.446 | 14.820 | 8.189 | 1.00 | 13.23 | H |
| ATOM | 2771 | CA | TYR | H | 147 | 13.053 | 15.561 | 7.101 | 1.00 | 12.47 | H |
| ATOM | 2772 | CB | TYR | H | 147 | 14.583 | 15.453 | 7.132 | 1.00 | 11.43 | H |
| ATOM | 2773 | CG | TYR | H | 147 | 15.139 | 14.070 | 6.848 | 1.00 | 10.78 | H |
| ATOM | 2774 | CD1 | TYR | H | 147 | 15.401 | 13.177 | 7.881 | 1.00 | 9.29 | H |
| ATOM | 2775 | CE1 | TYR | H | 147 | 15.965 | 11.924 | 7.630 | 1.00 | 10.28 | H |
| ATOM | 2776 | CD2 | TYR | H | 147 | 15.442 | 13.679 | 5.546 | 1.00 | 9.05 | H |
| ATOM | 2777 | CE2 | TYR | H | 147 | 16.004 | 12.431 | 5.279 | 1.00 | 12.95 | H |
| ATOM | 2778 | CZ | TYR | H | 147 | 16.264 | 11.560 | 6.329 | 1.00 | 11.51 | H |
| ATOM | 2779 | OH | TYR | H | 147 | 16.831 | 10.339 | 6.066 | 1.00 | 12.01 | H |
| ATOM | 2780 | C | TYR | H | 147 | 12.536 | 15.035 | 5.777 | 1.00 | 13.27 | H |
| ATOM | 2781 | O | TYR | H | 147 | 12.044 | 13.914 | 5.693 | 1.00 | 12.89 | H |
| ATOM | 2782 | N | PHE | H | 148 | 12.653 | 15.859 | 4.745 | 1.00 | 13.28 | H |
| ATOM | 2783 | CA | PHE | H | 148 | 12.223 | 15.491 | 3.416 | 1.00 | 12.06 | H |
| ATOM | 2784 | CB | PHE | H | 148 | 10.698 | 15.574 | 3.288 | 1.00 | 11.34 | H |
| ATOM | 2785 | CG | PHE | H | 148 | 10.182 | 15.035 | 1.987 | 1.00 | 8.03 | H |
| ATOM | 2786 | CD1 | PHE | H | 148 | 10.058 | 15.861 | 0.873 | 1.00 | 10.89 | H |
| ATOM | 2787 | CD2 | PHE | H | 148 | 9.908 | 13.671 | 1.849 | 1.00 | 12.28 | H |
| ATOM | 2788 | CE1 | PHE | H | 148 | 9.678 | 15.335 | −0.372 | 1.00 | 11.27 | H |
| ATOM | 2789 | CE2 | PHE | H | 148 | 9.528 | 13.130 | 0.617 | 1.00 | 7.09 | H |
| ATOM | 2790 | CZ | PHE | H | 148 | 9.416 | 13.966 | −0.499 | 1.00 | 9.10 | H |
| ATOM | 2791 | C | PHE | H | 148 | 12.870 | 16.450 | 2.431 | 1.00 | 11.47 | H |
| ATOM | 2792 | O | PHE | H | 148 | 12.928 | 17.651 | 2.684 | 1.00 | 14.31 | H |
| ATOM | 2793 | N | PRO | H | 149 | 13.402 | 15.925 | 1.319 | 1.00 | 12.30 | H |
| ATOM | 2794 | CD | PRO | H | 149 | 13.812 | 16.739 | 0.155 | 1.00 | 12.78 | H |
| ATOM | 2795 | CA | PRO | H | 149 | 13.410 | 14.492 | 0.987 | 1.00 | 12.66 | H |
| ATOM | 2796 | CB | PRO | H | 149 | 13.220 | 14.503 | −0.517 | 1.00 | 12.80 | H |
| ATOM | 2797 | CG | PRO | H | 149 | 14.120 | 15.675 | −0.924 | 1.00 | 11.93 | H |
| ATOM | 2798 | C | PRO | H | 149 | 14.762 | 13.877 | 1.372 | 1.00 | 13.89 | H |
| ATOM | 2799 | O | PRO | H | 149 | 15.515 | 14.470 | 2.139 | 1.00 | 11.29 | H |
| ATOM | 2800 | N | GLU | H | 150 | 15.053 | 12.684 | 0.857 | 1.00 | 15.23 | H |
| ATOM | 2801 | CA | GLU | H | 150 | 16.349 | 12.045 | 1.091 | 1.00 | 16.13 | H |
| ATOM | 2802 | CB | GLU | H | 150 | 16.330 | 10.591 | 0.606 | 1.00 | 16.56 | H |
| ATOM | 2803 | CG | GLU | H | 150 | 15.570 | 9.622 | 1.501 | 1.00 | 17.35 | H |
| ATOM | 2804 | CD | GLU | H | 150 | 16.420 | 9.095 | 2.642 | 1.00 | 20.36 | H |
| ATOM | 2805 | OE1 | GLU | H | 150 | 16.959 | 9.911 | 3.418 | 1.00 | 25.44 | H |
| ATOM | 2806 | OE2 | GLU | H | 150 | 16.553 | 7.863 | 2.770 | 1.00 | 21.03 | H |
| ATOM | 2807 | C | GLU | H | 150 | 17.307 | 12.860 | 0.211 | 1.00 | 16.85 | H |
| ATOM | 2808 | O | GLU | H | 150 | 16.875 | 13.493 | −0.746 | 1.00 | 16.25 | H |
| ATOM | 2809 | N | PRO | H | 151 | 18.614 | 12.848 | 0.512 | 1.00 | 16.90 | H |
| ATOM | 2810 | CD | PRO | H | 151 | 19.651 | 13.300 | −0.437 | 1.00 | 18.46 | H |
| ATOM | 2811 | CA | PRO | H | 151 | 19.227 | 12.119 | 1.616 | 1.00 | 17.43 | H |
| ATOM | 2812 | CB | PRO | H | 151 | 20.340 | 11.362 | 0.918 | 1.00 | 16.34 | H |
| ATOM | 2813 | CG | PRO | H | 151 | 20.875 | 12.430 | −0.062 | 1.00 | 17.91 | H |
| ATOM | 2814 | C | PRO | H | 151 | 19.778 | 13.040 | 2.699 | 1.00 | 17.98 | H |
| ATOM | 2815 | O | PRO | H | 151 | 19.797 | 14.267 | 2.559 | 1.00 | 16.93 | H |
| ATOM | 2816 | N | VAL | H | 152 | 20.211 | 12.422 | 3.787 | 1.00 | 17.69 | H |
| ATOM | 2817 | CA | VAL | H | 152 | 20.826 | 13.129 | 4.893 | 1.00 | 18.22 | H |
| ATOM | 2818 | CB | VAL | H | 152 | 20.014 | 12.970 | 6.190 | 1.00 | 18.05 | H |
| ATOM | 2819 | CG1 | VAL | H | 152 | 20.895 | 13.222 | 7.390 | 1.00 | 22.33 | H |
| ATOM | 2820 | CG2 | VAL | H | 152 | 18.849 | 13.945 | 6.193 | 1.00 | 20.82 | H |
| ATOM | 2821 | C | VAL | H | 152 | 22.158 | 12.416 | 5.062 | 1.00 | 17.39 | H |
| ATOM | 2822 | O | VAL | H | 152 | 22.237 | 11.214 | 4.819 | 1.00 | 16.64 | H |
| ATOM | 2823 | N | THR | H | 153 | 23.210 | 13.146 | 5.414 | 1.00 | 15.58 | H |
| ATOM | 2824 | CA | THR | H | 153 | 24.494 | 12.492 | 5.657 | 1.00 | 15.88 | H |
| ATOM | 2825 | CB | THR | H | 153 | 25.644 | 13.017 | 4.753 | 1.00 | 14.92 | H |
| ATOM | 2826 | OG1 | THR | H | 153 | 25.839 | 14.414 | 4.971 | 1.00 | 19.26 | H |
| ATOM | 2827 | CG2 | THR | H | 153 | 25.332 | 12.762 | 3.296 | 1.00 | 19.20 | H |
| ATOM | 2828 | C | THR | H | 153 | 24.837 | 12.773 | 7.112 | 1.00 | 15.07 | H |
| ATOM | 2829 | O | THR | H | 153 | 24.423 | 13.792 | 7.675 | 1.00 | 14.44 | H |
| ATOM | 2830 | N | VAL | H | 154 | 25.574 | 11.859 | 7.725 | 1.00 | 13.17 | H |
| ATOM | 2831 | CA | VAL | H | 154 | 25.968 | 12.013 | 9.111 | 1.00 | 12.33 | H |
| ATOM | 2832 | CB | VAL | H | 154 | 25.080 | 11.157 | 10.067 | 1.00 | 14.77 | H |
| ATOM | 2833 | CG1 | VAL | H | 154 | 25.503 | 11.378 | 11.518 | 1.00 | 12.28 | H |
| ATOM | 2834 | CG2 | VAL | H | 154 | 23.607 | 11.506 | 9.888 | 1.00 | 12.05 | H |
| ATOM | 2835 | C | VAL | H | 154 | 27.406 | 11.555 | 9.283 | 1.00 | 15.09 | H |
| ATOM | 2836 | O | VAL | H | 154 | 27.806 | 10.521 | 8.748 | 1.00 | 14.53 | H |
| ATOM | 2837 | N | SER | H | 156 | 28.190 | 12.339 | 10.010 | 1.00 | 14.01 | H |
| ATOM | 2838 | CA | SER | H | 156 | 29.561 | 11.959 | 10.301 | 1.00 | 14.74 | H |
| ATOM | 2839 | CB | SER | H | 156 | 30.556 | 12.771 | 9.463 | 1.00 | 16.67 | H |
| ATOM | 2840 | OG | SER | H | 156 | 30.515 | 14.137 | 9.819 | 1.00 | 21.53 | H |
| ATOM | 2841 | C | SER | H | 156 | 29.735 | 12.259 | 11.791 | 1.00 | 14.91 | H |
| ATOM | 2842 | O | SER | H | 156 | 28.892 | 12.923 | 12.404 | 1.00 | 12.85 | H |
| ATOM | 2843 | N | TRP | H | 157 | 30.801 | 11.739 | 12.383 | 1.00 | 11.38 | H |
| ATOM | 2844 | CA | TRP | H | 157 | 31.055 | 11.972 | 13.799 | 1.00 | 11.89 | H |
| ATOM | 2845 | CB | TRP | H | 157 | 31.005 | 10.662 | 14.588 | 1.00 | 10.69 | H |
| ATOM | 2846 | CG | TRP | H | 157 | 29.607 | 10.199 | 14.850 | 1.00 | 11.63 | H |
| ATOM | 2847 | CD2 | TRP | H | 157 | 28.777 | 10.562 | 15.963 | 1.00 | 10.96 | H |
| ATOM | 2848 | CE2 | TRP | H | 157 | 27.529 | 9.935 | 15.774 | 1.00 | 11.97 | H |
| ATOM | 2849 | CE3 | TRP | H | 157 | 28.968 | 11.360 | 17.099 | 1.00 | 13.66 | H |
| ATOM | 2850 | CD1 | TRP | H | 157 | 28.851 | 9.392 | 14.057 | 1.00 | 10.90 | H |
| ATOM | 2851 | NE1 | TRP | H | 157 | 27.601 | 9.228 | 14.604 | 1.00 | 12.42 | H |
| ATOM | 2852 | CZ2 | TRP | H | 157 | 26.470 | 10.076 | 16.681 | 1.00 | 12.67 | H |
| ATOM | 2853 | CZ3 | TRP | H | 157 | 27.917 | 11.504 | 18.001 | 1.00 | 13.73 | H |
| ATOM | 2854 | CH2 | TRP | H | 157 | 26.680 | 10.860 | 17.784 | 1.00 | 12.64 | H |
| ATOM | 2855 | C | TRP | H | 157 | 32.398 | 12.650 | 14.000 | 1.00 | 12.45 | H |
| ATOM | 2856 | O | TRP | H | 157 | 33.422 | 12.224 | 13.446 | 1.00 | 8.89 | H |
| ATOM | 2857 | N | ASN | H | 162 | 32.380 | 13.710 | 14.800 | 1.00 | 12.31 | H |
| ATOM | 2858 | CA | ASN | H | 162 | 33.585 | 14.480 | 15.072 | 1.00 | 14.19 | H |
| ATOM | 2859 | CB | ASN | H | 162 | 34.515 | 13.705 | 16.010 | 1.00 | 15.19 | H |
| ATOM | 2860 | CG | ASN | H | 162 | 33.935 | 13.569 | 17.407 | 1.00 | 15.88 | H |
| ATOM | 2861 | OD1 | ASN | H | 162 | 33.013 | 14.293 | 17.773 | 1.00 | 20.20 | H |
| ATOM | 2862 | ND2 | ASN | H | 162 | 34.476 | 12.652 | 18.193 | 1.00 | 17.77 | H |
| ATOM | 2863 | C | ASN | H | 162 | 34.280 | 14.807 | 13.761 | 1.00 | 14.28 | H |
| ATOM | 2864 | O | ASN | H | 162 | 35.474 | 14.582 | 13.593 | 1.00 | 16.36 | H |
| ATOM | 2865 | N | SER | H | 163 | 33.496 | 15.322 | 12.824 | 1.00 | 14.32 | H |
| ATOM | 2866 | CA | SER | H | 163 | 33.995 | 15.710 | 11.516 | 1.00 | 17.04 | H |
| ATOM | 2867 | CB | SER | H | 163 | 34.855 | 16.974 | 11.642 | 1.00 | 18.82 | H |
| ATOM | 2868 | OG | SER | H | 163 | 34.112 | 18.023 | 12.245 | 1.00 | 18.91 | H |
| ATOM | 2869 | C | SER | H | 163 | 34.782 | 14.626 | 10.799 | 1.00 | 18.40 | H |
| ATOM | 2870 | O | SER | H | 163 | 35.768 | 14.918 | 10.125 | 1.00 | 19.25 | H |
| ATOM | 2871 | N | GLY | H | 164 | 34.348 | 13.376 | 10.945 | 1.00 | 19.23 | H |
| ATOM | 2872 | CA | GLY | H | 164 | 35.019 | 12.284 | 10.263 | 1.00 | 18.56 | H |
| ATOM | 2873 | C | GLY | H | 164 | 36.181 | 11.632 | 10.985 | 1.00 | 19.26 | H |
| ATOM | 2874 | O | GLY | H | 164 | 36.731 | 10.649 | 10.496 | 1.00 | 19.76 | H |
| ATOM | 2875 | N | ALA | H | 165 | 36.560 | 12.167 | 12.142 | 1.00 | 18.26 | H |
| ATOM | 2876 | CA | ALA | H | 165 | 37.665 | 11.599 | 12.905 | 1.00 | 18.38 | H |
| ATOM | 2877 | CB | ALA | H | 165 | 38.176 | 12.614 | 13.926 | 1.00 | 18.34 | H |
| ATOM | 2878 | C | ALA | H | 165 | 37.229 | 10.321 | 13.618 | 1.00 | 17.24 | H |
| ATOM | 2879 | O | ALA | H | 165 | 38.055 | 9.481 | 13.968 | 1.00 | 17.12 | H |
| ATOM | 2880 | N | LEU | H | 166 | 35.928 | 10.179 | 13.829 | 1.00 | 14.08 | H |
| ATOM | 2881 | CA | LEU | H | 166 | 35.399 | 9.010 | 14.513 | 1.00 | 14.48 | H |
| ATOM | 2882 | CB | LEU | H | 166 | 34.556 | 9.446 | 15.717 | 1.00 | 11.61 | H |
| ATOM | 2883 | CG | LEU | H | 166 | 33.762 | 8.369 | 16.463 | 1.00 | 12.69 | H |
| ATOM | 2884 | CD1 | LEU | H | 166 | 34.700 | 7.291 | 17.007 | 1.00 | 14.91 | H |
| ATOM | 2885 | CD2 | LEU | H | 166 | 32.986 | 9.020 | 17.591 | 1.00 | 12.32 | H |
| ATOM | 2886 | C | LEU | H | 166 | 34.563 | 8.154 | 13.571 | 1.00 | 14.22 | H |
| ATOM | 2887 | O | LEU | H | 166 | 33.472 | 8.552 | 13.169 | 1.00 | 13.67 | H |
| ATOM | 2888 | N | THR | H | 167 | 35.087 | 6.983 | 13.222 | 1.00 | 14.70 | H |
| ATOM | 2889 | CA | THR | H | 167 | 34.391 | 6.060 | 12.336 | 1.00 | 15.67 | H |
| ATOM | 2890 | CB | THR | H | 167 | 35.113 | 5.927 | 10.974 | 1.00 | 16.13 | H |
| ATOM | 2891 | OG1 | THR | H | 167 | 36.455 | 5.471 | 11.184 | 1.00 | 16.76 | H |
| ATOM | 2892 | CG2 | THR | H | 167 | 35.139 | 7.273 | 10.243 | 1.00 | 15.04 | H |
| ATOM | 2893 | C | THR | H | 167 | 34.254 | 4.664 | 12.950 | 1.00 | 17.29 | H |
| ATOM | 2894 | O | THR | H | 167 | 33.261 | 3.970 | 12.711 | 1.00 | 19.07 | H |
| ATOM | 2895 | N | SER | H | 168 | 35.238 | 4.247 | 13.741 | 1.00 | 17.71 | H |
| ATOM | 2896 | CA | SER | H | 168 | 35.180 | 2.921 | 14.358 | 1.00 | 19.50 | H |
| ATOM | 2897 | CB | SER | H | 168 | 36.451 | 2.631 | 15.168 | 1.00 | 20.38 | H |
| ATOM | 2898 | OG | SER | H | 168 | 37.607 | 2.728 | 14.354 | 1.00 | 30.46 | H |
| ATOM | 2899 | C | SER | H | 168 | 33.975 | 2.805 | 15.276 | 1.00 | 17.75 | H |
| ATOM | 2900 | O | SER | H | 168 | 33.763 | 3.657 | 16.135 | 1.00 | 17.52 | H |
| ATOM | 2901 | N | GLY | H | 169 | 33.192 | 1.745 | 15.091 | 1.00 | 16.87 | H |
| ATOM | 2902 | CA | GLY | H | 169 | 32.022 | 1.527 | 15.924 | 1.00 | 15.54 | H |
| ATOM | 2903 | C | GLY | H | 169 | 30.812 | 2.367 | 15.552 | 1.00 | 15.00 | H |
| ATOM | 2904 | O | GLY | H | 169 | 29.812 | 2.356 | 16.263 | 1.00 | 16.21 | H |
| ATOM | 2905 | N | VAL | H | 171 | 30.887 | 3.097 | 14.445 | 1.00 | 15.48 | H |
| ATOM | 2906 | CA | VAL | H | 171 | 29.758 | 3.925 | 14.037 | 1.00 | 14.29 | H |
| ATOM | 2907 | CB | VAL | H | 171 | 30.220 | 5.186 | 13.267 | 1.00 | 16.11 | H |
| ATOM | 2908 | CG1 | VAL | H | 171 | 28.994 | 5.965 | 12.765 | 1.00 | 13.40 | H |
| ATOM | 2909 | CG2 | VAL | H | 171 | 31.078 | 6.064 | 14.170 | 1.00 | 14.98 | H |
| ATOM | 2910 | C | VAL | H | 171 | 28.760 | 3.181 | 13.152 | 1.00 | 14.28 | H |
| ATOM | 2911 | O | VAL | H | 171 | 29.143 | 2.469 | 12.227 | 1.00 | 11.21 | H |
| ATOM | 2912 | N | HIS | H | 172 | 27.477 | 3.348 | 13.454 | 1.00 | 13.08 | H |
| ATOM | 2913 | CA | HIS | H | 172 | 26.416 | 2.738 | 12.662 | 1.00 | 13.48 | H |
| ATOM | 2914 | CB | HIS | H | 172 | 25.740 | 1.582 | 13.412 | 1.00 | 12.73 | H |
| ATOM | 2915 | CG | HIS | H | 172 | 26.612 | 0.381 | 13.596 | 1.00 | 14.40 | H |
| ATOM | 2916 | CD2 | HIS | H | 172 | 26.885 | −0.360 | 14.696 | 1.00 | 11.87 | H |
| ATOM | 2917 | ND1 | HIS | H | 172 | 27.306 | −0.204 | 12.557 | 1.00 | 12.66 | H |
| ATOM | 2918 | CE1 | HIS | H | 172 | 27.971 | −1.251 | 13.011 | 1.00 | 12.50 | H |
| ATOM | 2919 | NE2 | HIS | H | 172 | 27.731 | −1.368 | 14.305 | 1.00 | 12.90 | H |
| ATOM | 2920 | C | HIS | H | 172 | 25.364 | 3.791 | 12.331 | 1.00 | 12.05 | H |
| ATOM | 2921 | O | HIS | H | 172 | 24.666 | 4.287 | 13.211 | 1.00 | 13.96 | H |
| ATOM | 2922 | N | THR | H | 173 | 25.271 | 4.139 | 11.058 | 1.00 | 14.60 | H |
| ATOM | 2923 | CA | THR | H | 173 | 24.284 | 5.107 | 10.601 | 1.00 | 14.83 | H |
| ATOM | 2924 | CB | THR | H | 173 | 24.918 | 6.162 | 9.688 | 1.00 | 15.73 | H |
| ATOM | 2925 | OG1 | THR | H | 173 | 25.803 | 6.983 | 10.464 | 1.00 | 16.56 | H |
| ATOM | 2926 | CG2 | THR | H | 173 | 23.846 | 7.025 | 9.041 | 1.00 | 15.30 | H |
| ATOM | 2927 | C | THR | H | 173 | 23.281 | 4.260 | 9.837 | 1.00 | 15.05 | H |
| ATOM | 2928 | O | THR | H | 173 | 23.585 | 3.715 | 8.781 | 1.00 | 12.55 | H |
| ATOM | 2929 | N | PHE | H | 174 | 22.089 | 4.147 | 10.405 | 1.00 | 13.75 | H |
| ATOM | 2930 | CA | PHE | H | 174 | 21.020 | 3.326 | 9.859 | 1.00 | 12.90 | H |
| ATOM | 2931 | CB | PHE | H | 174 | 20.041 | 2.994 | 10.982 | 1.00 | 10.53 | H |
| ATOM | 2932 | CG | PHE | H | 174 | 20.660 | 2.227 | 12.119 | 1.00 | 15.20 | H |
| ATOM | 2933 | CD1 | PHE | H | 174 | 20.626 | 0.829 | 12.138 | 1.00 | 14.31 | H |
| ATOM | 2934 | CD2 | PHE | H | 174 | 21.278 | 2.898 | 13.170 | 1.00 | 12.93 | H |
| ATOM | 2935 | CE1 | PHE | H | 174 | 21.196 | 0.113 | 13.190 | 1.00 | 11.60 | H |
| ATOM | 2936 | CE2 | PHE | H | 174 | 21.853 | 2.187 | 14.229 | 1.00 | 15.10 | H |
| ATOM | 2937 | CZ | PHE | H | 174 | 21.810 | 0.792 | 14.237 | 1.00 | 14.16 | H |
| ATOM | 2938 | C | PHE | H | 174 | 20.236 | 3.911 | 8.694 | 1.00 | 14.70 | H |
| ATOM | 2939 | O | PHE | H | 174 | 20.174 | 5.134 | 8.517 | 1.00 | 12.77 | H |
| ATOM | 2940 | N | PRO | H | 175 | 19.638 | 3.032 | 7.868 | 1.00 | 13.06 | H |
| ATOM | 2941 | CD | PRO | H | 175 | 19.778 | 1.563 | 7.834 | 1.00 | 12.74 | H |
| ATOM | 2942 | CA | PRO | H | 175 | 18.843 | 3.512 | 6.732 | 1.00 | 13.12 | H |
| ATOM | 2943 | CB | PRO | H | 175 | 18.381 | 2.220 | 6.051 | 1.00 | 12.11 | H |
| ATOM | 2944 | CG | PRO | H | 175 | 19.479 | 1.237 | 6.379 | 1.00 | 12.16 | H |
| ATOM | 2945 | C | PRO | H | 175 | 17.660 | 4.255 | 7.347 | 1.00 | 12.29 | H |
| ATOM | 2946 | O | PRO | H | 175 | 17.167 | 3.862 | 8.401 | 1.00 | 10.39 | H |
| ATOM | 2947 | N | ALA | H | 176 | 17.209 | 5.319 | 6.698 | 1.00 | 13.31 | H |
| ATOM | 2948 | CA | ALA | H | 176 | 16.083 | 6.085 | 7.207 | 1.00 | 13.00 | H |
| ATOM | 2949 | CB | ALA | H | 176 | 15.956 | 7.397 | 6.437 | 1.00 | 15.06 | H |
| ATOM | 2950 | C | ALA | H | 176 | 14.788 | 5.294 | 7.072 | 1.00 | 14.60 | H |
| ATOM | 2951 | O | ALA | H | 176 | 14.712 | 4.361 | 6.278 | 1.00 | 14.47 | H |
| ATOM | 2952 | N | VAL | H | 177 | 13.785 | 5.645 | 7.872 | 1.00 | 13.35 | H |
| ATOM | 2953 | CA | VAL | H | 177 | 12.479 | 5.005 | 7.753 | 1.00 | 14.66 | H |
| ATOM | 2954 | CB | VAL | H | 177 | 11.974 | 4.367 | 9.070 | 1.00 | 14.49 | H |
| ATOM | 2955 | CG1 | VAL | H | 177 | 12.847 | 3.169 | 9.437 | 1.00 | 16.93 | H |
| ATOM | 2956 | CG2 | VAL | H | 177 | 11.953 | 5.395 | 10.183 | 1.00 | 15.16 | H |
| ATOM | 2957 | C | VAL | H | 177 | 11.534 | 6.128 | 7.354 | 1.00 | 15.46 | H |
| ATOM | 2958 | O | VAL | H | 177 | 11.784 | 7.301 | 7.654 | 1.00 | 13.14 | H |
| ATOM | 2959 | N | LEU | H | 178 | 10.471 | 5.771 | 6.648 | 1.00 | 14.85 | H |
| ATOM | 2960 | CA | LEU | H | 178 | 9.485 | 6.738 | 6.196 | 1.00 | 15.30 | H |
| ATOM | 2961 | CB | LEU | H | 178 | 9.052 | 6.410 | 4.760 | 1.00 | 17.10 | H |
| ATOM | 2962 | CG | LEU | H | 178 | 8.030 | 7.343 | 4.093 | 1.00 | 18.90 | H |
| ATOM | 2963 | CD1 | LEU | H | 178 | 8.580 | 8.766 | 4.052 | 1.00 | 15.05 | H |
| ATOM | 2964 | CD2 | LEU | H | 178 | 7.721 | 6.842 | 2.685 | 1.00 | 19.03 | H |
| ATOM | 2965 | C | LEU | H | 178 | 8.307 | 6.633 | 7.150 | 1.00 | 14.48 | H |
| ATOM | 2966 | O | LEU | H | 178 | 7.633 | 5.607 | 7.202 | 1.00 | 15.78 | H |
| ATOM | 2967 | N | GLN | H | 179 | 8.072 | 7.694 | 7.911 | 1.00 | 14.63 | H |
| ATOM | 2968 | CA | GLN | H | 179 | 6.988 | 7.730 | 8.892 | 1.00 | 14.51 | H |
| ATOM | 2969 | CB | GLN | H | 179 | 7.279 | 8.831 | 9.909 | 1.00 | 12.55 | H |
| ATOM | 2970 | CG | GLN | H | 179 | 8.689 | 8.728 | 10.438 | 1.00 | 14.19 | H |
| ATOM | 2971 | CD | GLN | H | 179 | 9.108 | 9.921 | 11.255 | 1.00 | 17.73 | H |
| ATOM | 2972 | OE1 | GLN | H | 179 | 8.993 | 9.922 | 12.482 | 1.00 | 18.23 | H |
| ATOM | 2973 | NE2 | GLN | H | 179 | 9.593 | 10.956 | 10.576 | 1.00 | 12.19 | H |
| ATOM | 2974 | C | GLN | H | 179 | 5.615 | 7.943 | 8.258 | 1.00 | 11.65 | H |
| ATOM | 2975 | O | GLN | H | 179 | 5.513 | 8.279 | 7.078 | 1.00 | 11.46 | H |
| ATOM | 2976 | N | SER | H | 180 | 4.569 | 7.735 | 9.051 | 1.00 | 10.53 | H |
| ATOM | 2977 | CA | SER | H | 180 | 3.199 | 7.897 | 8.576 | 1.00 | 12.42 | H |
| ATOM | 2978 | CB | SER | H | 180 | 2.198 | 7.558 | 9.689 | 1.00 | 11.61 | H |
| ATOM | 2979 | OG | SER | H | 180 | 2.267 | 8.487 | 10.755 | 1.00 | 14.08 | H |
| ATOM | 2980 | C | SER | H | 180 | 2.931 | 9.311 | 8.072 | 1.00 | 12.55 | H |
| ATOM | 2981 | O | SER | H | 180 | 2.060 | 9.518 | 7.241 | 1.00 | 15.38 | H |
| ATOM | 2982 | N | SER | H | 182 | 3.675 | 10.285 | 8.584 | 1.00 | 10.64 | H |
| ATOM | 2983 | CA | SER | H | 182 | 3.508 | 11.672 | 8.165 | 1.00 | 10.68 | H |
| ATOM | 2984 | CB | SER | H | 182 | 4.228 | 12.605 | 9.139 | 1.00 | 10.83 | H |
| ATOM | 2985 | OG | SER | H | 182 | 5.621 | 12.341 | 9.121 | 1.00 | 12.18 | H |
| ATOM | 2986 | C | SER | H | 182 | 4.080 | 11.895 | 6.764 | 1.00 | 9.97 | H |
| ATOM | 2987 | O | SER | H | 182 | 3.802 | 12.912 | 6.135 | 1.00 | 10.75 | H |
| ATOM | 2988 | N | GLY | H | 183 | 4.874 | 10.940 | 6.280 | 1.00 | 9.39 | H |
| ATOM | 2989 | CA | GLY | H | 183 | 5.492 | 11.078 | 4.969 | 1.00 | 7.89 | H |
| ATOM | 2990 | C | GLY | H | 183 | 6.880 | 11.710 | 5.069 | 1.00 | 11.90 | H |
| ATOM | 2991 | O | GLY | H | 183 | 7.529 | 11.995 | 4.057 | 1.00 | 10.24 | H |
| ATOM | 2992 | N | LEU | H | 184 | 7.334 | 11.951 | 6.293 | 1.00 | 9.48 | H |
| ATOM | 2993 | CA | LEU | H | 184 | 8.655 | 12.528 | 6.502 | 1.00 | 12.10 | H |
| ATOM | 2994 | CB | LEU | H | 184 | 8.612 | 13.613 | 7.591 | 1.00 | 11.31 | H |
| ATOM | 2995 | CG | LEU | H | 184 | 7.671 | 14.811 | 7.396 | 1.00 | 9.98 | H |
| ATOM | 2996 | CD1 | LEU | H | 184 | 7.778 | 15.740 | 8.607 | 1.00 | 10.07 | H |
| ATOM | 2997 | CD2 | LEU | H | 184 | 8.029 | 15.555 | 6.116 | 1.00 | 11.93 | H |
| ATOM | 2998 | C | LEU | H | 184 | 9.595 | 11.393 | 6.930 | 1.00 | 13.12 | H |
| ATOM | 2999 | O | LEU | H | 184 | 9.163 | 10.397 | 7.522 | 1.00 | 11.68 | H |
| ATOM | 3000 | N | TYR | H | 185 | 10.875 | 11.547 | 6.612 | 1.00 | 12.41 | H |
| ATOM | 3001 | CA | TYR | H | 185 | 11.880 | 10.548 | 6.957 | 1.00 | 11.99 | H |
| ATOM | 3002 | CB | TYR | H | 185 | 13.015 | 10.577 | 5.942 | 1.00 | 9.63 | H |
| ATOM | 3003 | CG | TYR | H | 185 | 12.610 | 10.095 | 4.574 | 1.00 | 12.86 | H |
| ATOM | 3004 | CD1 | TYR | H | 185 | 12.583 | 8.731 | 4.271 | 1.00 | 8.00 | H |
| ATOM | 3005 | CE1 | TYR | H | 185 | 12.198 | 8.283 | 3.002 | 1.00 | 11.15 | H |
| ATOM | 3006 | CD2 | TYR | H | 185 | 12.242 | 11.001 | 3.582 | 1.00 | 9.43 | H |
| ATOM | 3007 | CE2 | TYR | H | 185 | 11.855 | 10.566 | 2.315 | 1.00 | 13.41 | H |
| ATOM | 3008 | CZ | TYR | H | 185 | 11.837 | 9.212 | 2.033 | 1.00 | 11.47 | H |
| ATOM | 3009 | OH | TYR | H | 185 | 11.469 | 8.795 | 0.783 | 1.00 | 14.63 | H |
| ATOM | 3010 | C | TYR | H | 185 | 12.466 | 10.765 | 8.341 | 1.00 | 12.05 | H |
| ATOM | 3011 | O | TYR | H | 185 | 12.344 | 11.840 | 8.924 | 1.00 | 11.72 | H |
| ATOM | 3012 | N | SER | H | 186 | 13.119 | 9.729 | 8.845 | 1.00 | 12.44 | H |
| ATOM | 3013 | CA | SER | H | 186 | 13.764 | 9.772 | 10.140 | 1.00 | 14.74 | H |
| ATOM | 3014 | CB | SER | H | 186 | 12.761 | 9.468 | 11.248 | 1.00 | 16.78 | H |
| ATOM | 3015 | OG | SER | H | 186 | 13.375 | 9.578 | 12.519 | 1.00 | 18.90 | H |
| ATOM | 3016 | C | SER | H | 186 | 14.876 | 8.740 | 10.177 | 1.00 | 16.16 | H |
| ATOM | 3017 | O | SER | H | 186 | 14.691 | 7.601 | 9.736 | 1.00 | 15.69 | H |
| ATOM | 3018 | N | LEU | H | 187 | 16.040 | 9.137 | 10.680 | 1.00 | 16.44 | H |
| ATOM | 3019 | CA | LEU | H | 187 | 17.147 | 8.201 | 10.782 | 1.00 | 16.87 | H |
| ATOM | 3020 | CB | LEU | H | 187 | 18.075 | 8.291 | 9.559 | 1.00 | 14.28 | H |
| ATOM | 3021 | CG | LEU | H | 187 | 19.212 | 9.287 | 9.296 | 1.00 | 19.24 | H |
| ATOM | 3022 | CD1 | LEU | H | 187 | 20.284 | 9.214 | 10.375 | 1.00 | 16.48 | H |
| ATOM | 3023 | CD2 | LEU | H | 187 | 19.834 | 8.941 | 7.930 | 1.00 | 15.16 | H |
| ATOM | 3024 | C | LEU | H | 187 | 17.937 | 8.382 | 12.070 | 1.00 | 14.24 | H |
| ATOM | 3025 | O | LEU | H | 187 | 17.868 | 9.421 | 12.724 | 1.00 | 15.63 | H |
| ATOM | 3026 | N | SER | H | 188 | 18.659 | 7.340 | 12.451 | 1.00 | 12.48 | H |
| ATOM | 3027 | CA | SER | H | 188 | 19.469 | 7.407 | 13.648 | 1.00 | 13.75 | H |
| ATOM | 3028 | CB | SER | H | 188 | 18.929 | 6.456 | 14.720 | 1.00 | 12.20 | H |
| ATOM | 3029 | OG | SER | H | 188 | 19.109 | 5.101 | 14.346 | 1.00 | 18.39 | H |
| ATOM | 3030 | C | SER | H | 188 | 20.898 | 7.035 | 13.280 | 1.00 | 14.03 | H |
| ATOM | 3031 | O | SER | H | 188 | 21.142 | 6.340 | 12.293 | 1.00 | 12.35 | H |
| ATOM | 3032 | N | SER | H | 189 | 21.836 | 7.546 | 14.061 | 1.00 | 13.43 | H |
| ATOM | 3033 | CA | SER | H | 189 | 23.249 | 7.259 | 13.880 | 1.00 | 14.59 | H |
| ATOM | 3034 | CB | SER | H | 189 | 23.988 | 8.440 | 13.249 | 1.00 | 12.93 | H |
| ATOM | 3035 | OG | SER | H | 189 | 25.338 | 8.091 | 12.988 | 1.00 | 15.58 | H |
| ATOM | 3036 | C | SER | H | 189 | 23.740 | 7.037 | 15.300 | 1.00 | 12.88 | H |
| ATOM | 3037 | O | SER | H | 189 | 23.433 | 7.825 | 16.197 | 1.00 | 12.40 | H |
| ATOM | 3038 | N | VAL | H | 190 | 24.481 | 5.957 | 15.506 | 1.00 | 12.24 | H |
| ATOM | 3039 | CA | VAL | H | 190 | 24.973 | 5.633 | 16.833 | 1.00 | 11.28 | H |
| ATOM | 3040 | CB | VAL | H | 190 | 24.154 | 4.486 | 17.468 | 1.00 | 10.78 | H |
| ATOM | 3041 | CG1 | VAL | H | 190 | 22.657 | 4.805 | 17.420 | 1.00 | 10.28 | H |
| ATOM | 3042 | CG2 | VAL | H | 190 | 24.454 | 3.179 | 16.744 | 1.00 | 8.15 | H |
| ATOM | 3043 | C | VAL | H | 190 | 26.417 | 5.177 | 16.778 | 1.00 | 13.37 | H |
| ATOM | 3044 | O | VAL | H | 190 | 26.949 | 4.869 | 15.709 | 1.00 | 11.55 | H |
| ATOM | 3045 | N | VAL | H | 191 | 27.040 | 5.122 | 17.947 | 1.00 | 14.34 | H |
| ATOM | 3046 | CA | VAL | H | 191 | 28.414 | 4.673 | 18.058 | 1.00 | 13.48 | H |
| ATOM | 3047 | CB | VAL | H | 191 | 29.410 | 5.845 | 17.831 | 1.00 | 14.93 | H |
| ATOM | 3048 | CG1 | VAL | H | 191 | 29.159 | 6.943 | 18.851 | 1.00 | 16.88 | H |
| ATOM | 3049 | CG2 | VAL | H | 191 | 30.856 | 5.341 | 17.903 | 1.00 | 10.61 | H |
| ATOM | 3050 | C | VAL | H | 191 | 28.614 | 4.087 | 19.449 | 1.00 | 13.80 | H |
| ATOM | 3051 | O | VAL | H | 191 | 28.017 | 4.545 | 20.422 | 1.00 | 14.44 | H |
| ATOM | 3052 | N | THR | H | 192 | 29.406 | 3.029 | 19.534 | 1.00 | 14.76 | H |
| ATOM | 3053 | CA | THR | H | 192 | 29.695 | 2.441 | 20.832 | 1.00 | 17.33 | H |
| ATOM | 3054 | CB | THR | H | 192 | 29.538 | 0.908 | 20.827 | 1.00 | 17.10 | H |
| ATOM | 3055 | OG1 | THR | H | 192 | 30.366 | 0.342 | 19.812 | 1.00 | 15.81 | H |
| ATOM | 3056 | CG2 | THR | H | 192 | 28.081 | 0.531 | 20.579 | 1.00 | 18.14 | H |
| ATOM | 3057 | C | THR | H | 192 | 31.141 | 2.837 | 21.117 | 1.00 | 19.24 | H |
| ATOM | 3058 | O | THR | H | 192 | 31.966 | 2.880 | 20.207 | 1.00 | 19.20 | H |
| ATOM | 3059 | N | VAL | H | 193 | 31.430 | 3.153 | 22.373 | 1.00 | 20.59 | H |
| ATOM | 3060 | CA | VAL | H | 193 | 32.760 | 3.586 | 22.782 | 1.00 | 23.39 | H |
| ATOM | 3061 | CB | VAL | H | 193 | 32.874 | 5.131 | 22.758 | 1.00 | 23.69 | H |
| ATOM | 3062 | CG1 | VAL | H | 193 | 32.637 | 5.662 | 21.358 | 1.00 | 23.98 | H |
| ATOM | 3063 | CG2 | VAL | H | 193 | 31.857 | 5.742 | 23.728 | 1.00 | 23.50 | H |
| ATOM | 3064 | C | VAL | H | 193 | 33.032 | 3.135 | 24.214 | 1.00 | 25.63 | H |
| ATOM | 3065 | O | VAL | H | 193 | 32.106 | 2.786 | 24.952 | 1.00 | 22.71 | H |
| ATOM | 3066 | N | PRO | H | 194 | 34.310 | 3.131 | 24.624 | 1.00 | 28.39 | H |
| ATOM | 3067 | CD | PRO | H | 194 | 35.536 | 3.320 | 23.828 | 1.00 | 29.91 | H |
| ATOM | 3068 | CA | PRO | H | 194 | 34.636 | 2.719 | 25.992 | 1.00 | 29.54 | H |
| ATOM | 3069 | CB | PRO | H | 194 | 36.159 | 2.820 | 26.027 | 1.00 | 29.18 | H |
| ATOM | 3070 | CG | PRO | H | 194 | 36.548 | 2.512 | 24.610 | 1.00 | 29.53 | H |
| ATOM | 3071 | C | PRO | H | 194 | 33.969 | 3.699 | 26.951 | 1.00 | 29.60 | H |
| ATOM | 3072 | O | PRO | H | 194 | 34.024 | 4.911 | 26.747 | 1.00 | 30.65 | H |
| ATOM | 3073 | N | SER | H | 195 | 33.324 | 3.182 | 27.987 | 1.00 | 30.73 | H |
| ATOM | 3074 | CA | SER | H | 195 | 32.659 | 4.045 | 28.951 | 1.00 | 32.14 | H |
| ATOM | 3075 | CB | SER | H | 195 | 32.070 | 3.206 | 30.076 | 1.00 | 31.17 | H |
| ATOM | 3076 | OG | SER | H | 195 | 31.167 | 2.246 | 29.563 | 1.00 | 33.28 | H |
| ATOM | 3077 | C | SER | H | 195 | 33.626 | 5.077 | 29.529 | 1.00 | 33.11 | H |
| ATOM | 3078 | O | SER | H | 195 | 33.240 | 6.208 | 29.827 | 1.00 | 32.61 | H |
| ATOM | 3079 | N | SER | H | 196 | 34.885 | 4.686 | 29.679 | 1.00 | 32.87 | H |
| ATOM | 3080 | CA | SER | H | 196 | 35.888 | 5.588 | 30.229 | 1.00 | 35.42 | H |
| ATOM | 3081 | CB | SER | H | 196 | 37.174 | 4.816 | 30.529 | 1.00 | 33.36 | H |
| ATOM | 3082 | OG | SER | H | 196 | 37.624 | 4.119 | 29.384 | 1.00 | 35.85 | H |
| ATOM | 3083 | C | SER | H | 196 | 36.195 | 6.770 | 29.311 | 1.00 | 35.88 | H |
| ATOM | 3084 | O | SER | H | 196 | 36.732 | 7.784 | 29.753 | 1.00 | 36.90 | H |
| ATOM | 3085 | N | SER | H | 197 | 35.847 | 6.648 | 28.036 | 1.00 | 36.60 | H |
| ATOM | 3086 | CA | SER | H | 197 | 36.114 | 7.723 | 27.088 | 1.00 | 37.54 | H |
| ATOM | 3087 | CB | SER | H | 197 | 36.107 | 7.182 | 25.660 | 1.00 | 37.36 | H |
| ATOM | 3088 | OG | SER | H | 197 | 34.783 | 6.915 | 25.237 | 1.00 | 40.28 | H |
| ATOM | 3089 | C | SER | H | 197 | 35.100 | 8.858 | 27.202 | 1.00 | 37.96 | H |
| ATOM | 3090 | O | SER | H | 197 | 35.295 | 9.928 | 26.627 | 1.00 | 39.43 | H |
| ATOM | 3091 | N | LEU | H | 198 | 34.024 | 8.629 | 27.946 | 1.00 | 37.12 | H |
| ATOM | 3092 | CA | LEU | H | 198 | 32.995 | 9.648 | 28.110 | 1.00 | 38.71 | H |
| ATOM | 3093 | CB | LEU | H | 198 | 31.768 | 9.066 | 28.818 | 1.00 | 35.78 | H |
| ATOM | 3094 | CG | LEU | H | 198 | 31.031 | 7.922 | 28.112 | 1.00 | 33.84 | H |
| ATOM | 3095 | CD1 | LEU | H | 198 | 29.796 | 7.551 | 28.907 | 1.00 | 31.98 | H |
| ATOM | 3096 | CD2 | LEU | H | 198 | 30.641 | 8.339 | 26.703 | 1.00 | 31.97 | H |
| ATOM | 3097 | C | LEU | H | 198 | 33.510 | 10.850 | 28.890 | 1.00 | 40.83 | H |
| ATOM | 3098 | O | LEU | H | 198 | 32.825 | 11.868 | 29.002 | 1.00 | 42.63 | H |
| ATOM | 3099 | N | GLY | H | 199 | 34.720 | 10.729 | 29.427 | 1.00 | 41.23 | H |
| ATOM | 3100 | CA | GLY | H | 199 | 35.300 | 11.818 | 30.189 | 1.00 | 41.10 | H |
| ATOM | 3101 | C | GLY | H | 199 | 36.580 | 12.346 | 29.575 | 1.00 | 41.10 | H |
| ATOM | 3102 | O | GLY | H | 199 | 37.117 | 13.356 | 30.020 | 1.00 | 41.28 | H |
| ATOM | 3103 | N | THR | H | 200 | 37.076 | 11.666 | 28.550 | 1.00 | 40.87 | H |
| ATOM | 3104 | CA | THR | H | 200 | 38.298 | 12.101 | 27.887 | 1.00 | 41.83 | H |
| ATOM | 3105 | CB | THR | H | 200 | 39.389 | 11.019 | 27.958 | 1.00 | 42.40 | H |
| ATOM | 3106 | OG1 | THR | H | 200 | 38.933 | 9.835 | 27.292 | 1.00 | 43.73 | H |
| ATOM | 3107 | CG2 | THR | H | 200 | 39.716 | 10.693 | 29.403 | 1.00 | 42.23 | H |
| ATOM | 3108 | C | THR | H | 200 | 38.047 | 12.419 | 26.418 | 1.00 | 41.20 | H |
| ATOM | 3109 | O | THR | H | 200 | 38.989 | 12.649 | 25.660 | 1.00 | 42.10 | H |
| ATOM | 3110 | N | GLN | H | 203 | 36.780 | 12.441 | 26.016 | 1.00 | 38.65 | H |
| ATOM | 3111 | CA | GLN | H | 203 | 36.448 | 12.711 | 24.624 | 1.00 | 34.88 | H |
| ATOM | 3112 | CB | GLN | H | 203 | 36.596 | 11.423 | 23.811 | 1.00 | 37.31 | H |
| ATOM | 3113 | CG | GLN | H | 203 | 36.271 | 11.555 | 22.325 | 1.00 | 40.59 | H |
| ATOM | 3114 | CD | GLN | H | 203 | 37.321 | 12.338 | 21.562 | 1.00 | 43.51 | H |
| ATOM | 3115 | OE1 | GLN | H | 203 | 37.044 | 13.407 | 21.014 | 1.00 | 44.26 | H |
| ATOM | 3116 | NE2 | GLN | H | 203 | 38.539 | 11.807 | 21.523 | 1.00 | 44.42 | H |
| ATOM | 3117 | C | GLN | H | 203 | 35.039 | 13.278 | 24.439 | 1.00 | 32.44 | H |
| ATOM | 3118 | O | GLN | H | 203 | 34.096 | 12.892 | 25.134 | 1.00 | 30.75 | H |
| ATOM | 3119 | N | THR | H | 205 | 34.904 | 14.207 | 23.501 | 1.00 | 28.52 | H |
| ATOM | 3120 | CA | THR | H | 205 | 33.612 | 14.793 | 23.205 | 1.00 | 25.37 | H |
| ATOM | 3121 | CB | THR | H | 205 | 33.733 | 16.295 | 22.922 | 1.00 | 27.55 | H |
| ATOM | 3122 | OG1 | THR | H | 205 | 34.601 | 16.498 | 21.802 | 1.00 | 30.58 | H |
| ATOM | 3123 | CG2 | THR | H | 205 | 34.298 | 17.018 | 24.136 | 1.00 | 28.30 | H |
| ATOM | 3124 | C | THR | H | 205 | 33.081 | 14.086 | 21.956 | 1.00 | 21.82 | H |
| ATOM | 3125 | O | THR | H | 205 | 33.853 | 13.611 | 21.128 | 1.00 | 21.85 | H |
| ATOM | 3126 | N | TYR | H | 206 | 31.765 | 14.011 | 21.831 | 1.00 | 17.40 | H |
| ATOM | 3127 | CA | TYR | H | 206 | 31.141 | 13.354 | 20.690 | 1.00 | 14.89 | H |
| ATOM | 3128 | CB | TYR | H | 206 | 30.458 | 12.067 | 21.154 | 1.00 | 13.89 | H |
| ATOM | 3129 | CG | TYR | H | 206 | 31.448 | 11.051 | 21.687 | 1.00 | 15.60 | H |
| ATOM | 3130 | CD1 | TYR | H | 206 | 32.280 | 10.342 | 20.819 | 1.00 | 16.07 | H |
| ATOM | 3131 | CE1 | TYR | H | 206 | 33.250 | 9.462 | 21.300 | 1.00 | 18.69 | H |
| ATOM | 3132 | CD2 | TYR | H | 206 | 31.604 | 10.850 | 23.062 | 1.00 | 15.77 | H |
| ATOM | 3133 | CE2 | TYR | H | 206 | 32.576 | 9.968 | 23.558 | 1.00 | 19.74 | H |
| ATOM | 3134 | CZ | TYR | H | 206 | 33.396 | 9.281 | 22.666 | 1.00 | 19.58 | H |
| ATOM | 3135 | OH | TYR | H | 206 | 34.379 | 8.435 | 23.128 | 1.00 | 21.91 | H |
| ATOM | 3136 | C | TYR | H | 206 | 30.151 | 14.304 | 20.025 | 1.00 | 15.00 | H |
| ATOM | 3137 | O | TYR | H | 206 | 29.209 | 14.801 | 20.648 | 1.00 | 15.44 | H |
| ATOM | 3138 | N | ILE | H | 207 | 30.393 | 14.562 | 18.749 | 1.00 | 13.97 | H |
| ATOM | 3139 | CA | ILE | H | 207 | 29.569 | 15.475 | 17.989 | 1.00 | 14.80 | H |
| ATOM | 3140 | CB | ILE | H | 207 | 30.355 | 16.770 | 17.663 | 1.00 | 15.14 | H |
| ATOM | 3141 | CG2 | ILE | H | 207 | 29.506 | 17.698 | 16.797 | 1.00 | 16.39 | H |
| ATOM | 3142 | CG1 | ILE | H | 207 | 30.775 | 17.463 | 18.963 | 1.00 | 20.46 | H |
| ATOM | 3143 | CD1 | ILE | H | 207 | 31.654 | 18.683 | 18.751 | 1.00 | 21.26 | H |
| ATOM | 3144 | C | ILE | H | 207 | 29.134 | 14.860 | 16.676 | 1.00 | 14.48 | H |
| ATOM | 3145 | O | ILE | H | 207 | 29.970 | 14.443 | 15.877 | 1.00 | 12.08 | H |
| ATOM | 3146 | N | CYS | H | 208 | 27.830 | 14.803 | 16.440 | 1.00 | 14.19 | H |
| ATOM | 3147 | CA | CYS | H | 208 | 27.376 | 14.269 | 15.174 | 1.00 | 16.35 | H |
| ATOM | 3148 | C | CYS | H | 208 | 27.163 | 15.464 | 14.246 | 1.00 | 15.65 | H |
| ATOM | 3149 | O | CYS | H | 208 | 26.555 | 16.467 | 14.631 | 1.00 | 14.46 | H |
| ATOM | 3150 | CB | CYS | H | 208 | 26.086 | 13.451 | 15.332 | 1.00 | 18.24 | H |
| ATOM | 3151 | SG | CYS | H | 208 | 24.614 | 14.382 | 15.840 | 1.00 | 23.36 | H |
| ATOM | 3152 | N | ASN | H | 209 | 27.697 | 15.346 | 13.035 | 1.00 | 15.36 | H |
| ATOM | 3153 | CA | ASN | H | 209 | 27.601 | 16.388 | 12.023 | 1.00 | 15.16 | H |
| ATOM | 3154 | CB | ASN | H | 209 | 28.941 | 16.554 | 11.309 | 1.00 | 13.23 | H |
| ATOM | 3155 | CG | ASN | H | 209 | 30.117 | 16.485 | 12.263 | 1.00 | 16.39 | H |
| ATOM | 3156 | OD1 | ASN | H | 209 | 30.801 | 15.469 | 12.345 | 1.00 | 17.17 | H |
| ATOM | 3157 | ND2 | ASN | H | 209 | 30.348 | 17.564 | 12.996 | 1.00 | 15.81 | H |
| ATOM | 3158 | C | ASN | H | 209 | 26.549 | 15.955 | 11.023 | 1.00 | 14.07 | H |
| ATOM | 3159 | O | ASN | H | 209 | 26.777 | 15.045 | 10.233 | 1.00 | 12.56 | H |
| ATOM | 3160 | N | VAL | H | 210 | 25.403 | 16.622 | 11.056 | 1.00 | 12.76 | H |
| ATOM | 3161 | CA | VAL | H | 210 | 24.300 | 16.279 | 10.177 | 1.00 | 12.88 | H |
| ATOM | 3162 | CB | VAL | H | 210 | 22.986 | 16.185 | 10.988 | 1.00 | 13.26 | H |
| ATOM | 3163 | CG1 | VAL | H | 210 | 21.806 | 15.914 | 10.070 | 1.00 | 11.04 | H |
| ATOM | 3164 | CG2 | VAL | H | 210 | 23.114 | 15.083 | 12.028 | 1.00 | 9.09 | H |
| ATOM | 3165 | C | VAL | H | 210 | 24.149 | 17.291 | 9.057 | 1.00 | 14.04 | H |
| ATOM | 3166 | O | VAL | H | 210 | 24.147 | 18.503 | 9.290 | 1.00 | 14.34 | H |
| ATOM | 3167 | N | ASN | H | 211 | 24.006 | 16.775 | 7.843 | 1.00 | 12.99 | H |
| ATOM | 3168 | CA | ASN | H | 211 | 23.867 | 17.605 | 6.663 | 1.00 | 15.10 | H |
| ATOM | 3169 | CB | ASN | H | 211 | 25.141 | 17.505 | 5.824 | 1.00 | 16.16 | H |
| ATOM | 3170 | CG | ASN | H | 211 | 25.205 | 18.554 | 4.735 | 1.00 | 20.86 | H |
| ATOM | 3171 | OD1 | ASN | H | 211 | 24.668 | 18.374 | 3.644 | 1.00 | 21.80 | H |
| ATOM | 3172 | ND2 | ASN | H | 211 | 25.858 | 19.666 | 5.035 | 1.00 | 22.71 | H |
| ATOM | 3173 | C | ASN | H | 211 | 22.658 | 17.215 | 5.810 | 1.00 | 14.13 | H |
| ATOM | 3174 | O | ASN | H | 211 | 22.561 | 16.092 | 5.320 | 1.00 | 11.98 | H |
| ATOM | 3175 | N | HIS | H | 212 | 21.732 | 18.149 | 5.648 | 1.00 | 14.66 | H |
| ATOM | 3176 | CA | HIS | H | 212 | 20.553 | 17.920 | 4.822 | 1.00 | 13.85 | H |
| ATOM | 3177 | CB | HIS | H | 212 | 19.292 | 17.901 | 5.688 | 1.00 | 13.98 | H |
| ATOM | 3178 | CG | HIS | H | 212 | 18.031 | 17.696 | 4.908 | 1.00 | 14.62 | H |
| ATOM | 3179 | CD2 | HIS | H | 212 | 16.876 | 18.402 | 4.882 | 1.00 | 11.94 | H |
| ATOM | 3180 | ND1 | HIS | H | 212 | 17.865 | 16.662 | 4.013 | 1.00 | 16.10 | H |
| ATOM | 3181 | CE1 | HIS | H | 212 | 16.664 | 16.744 | 3.468 | 1.00 | 10.41 | H |
| ATOM | 3182 | NE2 | HIS | H | 212 | 16.046 | 17.791 | 3.978 | 1.00 | 12.62 | H |
| ATOM | 3183 | C | HIS | H | 212 | 20.536 | 19.081 | 3.831 | 1.00 | 14.40 | H |
| ATOM | 3184 | O | HIS | H | 212 | 19.996 | 20.152 | 4.109 | 1.00 | 13.20 | H |
| ATOM | 3185 | N | LYS | H | 213 | 21.145 | 18.856 | 2.672 | 1.00 | 13.15 | H |
| ATOM | 3186 | CA | LYS | H | 213 | 21.269 | 19.893 | 1.655 | 1.00 | 14.16 | H |
| ATOM | 3187 | CB | LYS | H | 213 | 22.062 | 19.352 | 0.459 | 1.00 | 15.28 | H |
| ATOM | 3188 | CG | LYS | H | 213 | 22.463 | 20.429 | −0.544 | 1.00 | 15.87 | H |
| ATOM | 3189 | CD | LYS | H | 213 | 23.331 | 19.899 | −1.674 | 1.00 | 16.44 | H |
| ATOM | 3190 | CE | LYS | H | 213 | 23.877 | 21.056 | −2.510 | 1.00 | 17.95 | H |
| ATOM | 3191 | NZ | LYS | H | 213 | 24.697 | 20.631 | −3.696 | 1.00 | 15.63 | H |
| ATOM | 3192 | C | LYS | H | 213 | 19.992 | 20.573 | 1.154 | 1.00 | 14.43 | H |
| ATOM | 3193 | O | LYS | H | 213 | 19.960 | 21.790 | 0.997 | 1.00 | 13.23 | H |
| ATOM | 3194 | N | PRO | H | 214 | 18.924 | 19.800 | 0.910 | 1.00 | 12.41 | H |
| ATOM | 3195 | CD | PRO | H | 214 | 18.827 | 18.332 | 0.959 | 1.00 | 12.55 | H |
| ATOM | 3196 | CA | PRO | H | 214 | 17.675 | 20.388 | 0.417 | 1.00 | 12.68 | H |
| ATOM | 3197 | CB | PRO | H | 214 | 16.744 | 19.178 | 0.305 | 1.00 | 11.45 | H |
| ATOM | 3198 | CG | PRO | H | 214 | 17.695 | 18.064 | −0.010 | 1.00 | 13.32 | H |
| ATOM | 3199 | C | PRO | H | 214 | 17.091 | 21.488 | 1.279 | 1.00 | 13.16 | H |
| ATOM | 3200 | O | PRO | H | 214 | 16.541 | 22.464 | 0.767 | 1.00 | 14.68 | H |
| ATOM | 3201 | N | SER | H | 215 | 17.198 | 21.327 | 2.591 | 1.00 | 12.30 | H |
| ATOM | 3202 | CA | SER | H | 215 | 16.673 | 22.321 | 3.507 | 1.00 | 12.03 | H |
| ATOM | 3203 | CB | SER | H | 215 | 15.957 | 21.640 | 4.669 | 1.00 | 12.00 | H |
| ATOM | 3204 | OG | SER | H | 215 | 16.887 | 20.929 | 5.466 | 1.00 | 11.84 | H |
| ATOM | 3205 | C | SER | H | 215 | 17.794 | 23.188 | 4.068 | 1.00 | 13.50 | H |
| ATOM | 3206 | O | SER | H | 215 | 17.538 | 24.064 | 4.887 | 1.00 | 13.37 | H |
| ATOM | 3207 | N | ASN | H | 216 | 19.020 | 22.940 | 3.610 | 1.00 | 12.33 | H |
| ATOM | 3208 | CA | ASN | H | 216 | 20.217 | 23.641 | 4.089 | 1.00 | 14.11 | H |
| ATOM | 3209 | CB | ASN | H | 216 | 20.249 | 25.110 | 3.659 | 1.00 | 12.30 | H |
| ATOM | 3210 | CG | ASN | H | 216 | 21.583 | 25.794 | 4.006 | 1.00 | 14.08 | H |
| ATOM | 3211 | OD1 | ASN | H | 216 | 22.662 | 25.266 | 3.724 | 1.00 | 14.68 | H |
| ATOM | 3212 | ND2 | ASN | H | 216 | 21.506 | 26.969 | 4.615 | 1.00 | 10.56 | H |
| ATOM | 3213 | C | ASN | H | 216 | 20.295 | 23.557 | 5.605 | 1.00 | 15.47 | H |
| ATOM | 3214 | O | ASN | H | 216 | 20.540 | 24.550 | 6.290 | 1.00 | 16.57 | H |
| ATOM | 3215 | N | THR | H | 217 | 20.047 | 22.360 | 6.124 | 1.00 | 16.72 | H |
| ATOM | 3216 | CA | THR | H | 217 | 20.125 | 22.113 | 7.558 | 1.00 | 17.20 | H |
| ATOM | 3217 | CB | THR | H | 217 | 19.031 | 21.129 | 8.034 | 1.00 | 17.84 | H |
| ATOM | 3218 | OG1 | THR | H | 217 | 17.745 | 21.734 | 7.870 | 1.00 | 16.36 | H |
| ATOM | 3219 | CG2 | THR | H | 217 | 19.228 | 20.768 | 9.508 | 1.00 | 18.34 | H |
| ATOM | 3220 | C | THR | H | 217 | 21.494 | 21.487 | 7.810 | 1.00 | 17.22 | H |
| ATOM | 3221 | O | THR | H | 217 | 21.803 | 20.422 | 7.291 | 1.00 | 19.22 | H |
| ATOM | 3222 | N | LYS | H | 218 | 22.320 | 22.170 | 8.586 | 1.00 | 18.07 | H |
| ATOM | 3223 | CA | LYS | H | 218 | 23.651 | 21.678 | 8.904 | 1.00 | 19.21 | H |
| ATOM | 3224 | CB | LYS | H | 218 | 24.688 | 22.425 | 8.068 | 1.00 | 17.40 | H |
| ATOM | 3225 | CG | LYS | H | 218 | 24.373 | 22.281 | 6.592 | 1.00 | 18.14 | H |
| ATOM | 3226 | CD | LYS | H | 218 | 25.440 | 22.817 | 5.687 | 1.00 | 17.51 | H |
| ATOM | 3227 | CE | LYS | H | 218 | 25.076 | 22.513 | 4.248 | 1.00 | 15.40 | H |
| ATOM | 3228 | NZ | LYS | H | 218 | 26.091 | 23.015 | 3.314 | 1.00 | 16.98 | H |
| ATOM | 3229 | C | LYS | H | 218 | 23.859 | 21.875 | 10.392 | 1.00 | 19.04 | H |
| ATOM | 3230 | O | LYS | H | 218 | 24.134 | 22.976 | 10.861 | 1.00 | 21.71 | H |
| ATOM | 3231 | N | VAL | H | 219 | 23.683 | 20.787 | 11.125 | 1.00 | 18.14 | H |
| ATOM | 3232 | CA | VAL | H | 219 | 23.793 | 20.794 | 12.572 | 1.00 | 17.91 | H |
| ATOM | 3233 | CB | VAL | H | 219 | 22.482 | 20.264 | 13.213 | 1.00 | 17.02 | H |
| ATOM | 3234 | CG1 | VAL | H | 219 | 22.646 | 20.130 | 14.723 | 1.00 | 17.60 | H |
| ATOM | 3235 | CG2 | VAL | H | 219 | 21.328 | 21.194 | 12.885 | 1.00 | 17.46 | H |
| ATOM | 3236 | C | VAL | H | 219 | 24.945 | 19.944 | 13.089 | 1.00 | 17.19 | H |
| ATOM | 3237 | O | VAL | H | 219 | 25.188 | 18.839 | 12.600 | 1.00 | 16.56 | H |
| ATOM | 3238 | N | ASP | H | 220 | 25.645 | 20.473 | 14.084 | 1.00 | 16.07 | H |
| ATOM | 3239 | CA | ASP | H | 220 | 26.740 | 19.762 | 14.724 | 1.00 | 16.94 | H |
| ATOM | 3240 | CB | ASP | H | 220 | 28.028 | 20.581 | 14.670 | 1.00 | 17.63 | H |
| ATOM | 3241 | CG | ASP | H | 220 | 28.530 | 20.770 | 13.258 | 1.00 | 20.36 | H |
| ATOM | 3242 | OD1 | ASP | H | 220 | 28.731 | 19.754 | 12.564 | 1.00 | 20.83 | H |
| ATOM | 3243 | OD2 | ASP | H | 220 | 28.722 | 21.929 | 12.832 | 1.00 | 22.27 | H |
| ATOM | 3244 | C | ASP | H | 220 | 26.252 | 19.624 | 16.150 | 1.00 | 17.75 | H |
| ATOM | 3245 | O | ASP | H | 220 | 26.391 | 20.540 | 16.959 | 1.00 | 19.71 | H |
| ATOM | 3246 | N | LYS | H | 221 | 25.670 | 18.472 | 16.452 | 1.00 | 18.16 | H |
| ATOM | 3247 | CA | LYS | H | 221 | 25.100 | 18.229 | 17.770 | 1.00 | 17.27 | H |
| ATOM | 3248 | CB | LYS | H | 221 | 23.794 | 17.449 | 17.613 | 1.00 | 17.83 | H |
| ATOM | 3249 | CG | LYS | H | 221 | 23.029 | 17.211 | 18.897 | 1.00 | 22.03 | H |
| ATOM | 3250 | CD | LYS | H | 221 | 22.559 | 18.515 | 19.508 | 1.00 | 26.12 | H |
| ATOM | 3251 | CE | LYS | H | 221 | 21.686 | 18.256 | 20.722 | 1.00 | 31.10 | H |
| ATOM | 3252 | NZ | LYS | H | 221 | 21.413 | 19.498 | 21.502 | 1.00 | 34.16 | H |
| ATOM | 3253 | C | LYS | H | 221 | 26.027 | 17.484 | 18.715 | 1.00 | 17.28 | H |
| ATOM | 3254 | O | LYS | H | 221 | 26.423 | 16.346 | 18.450 | 1.00 | 15.86 | H |
| ATOM | 3255 | N | LYS | H | 222 | 26.365 | 18.130 | 19.824 | 1.00 | 17.69 | H |
| ATOM | 3256 | CA | LYS | H | 222 | 27.230 | 17.514 | 20.815 | 1.00 | 19.16 | H |
| ATOM | 3257 | CB | LYS | H | 222 | 27.888 | 18.581 | 21.692 | 1.00 | 20.64 | H |
| ATOM | 3258 | CG | LYS | H | 222 | 28.819 | 18.013 | 22.759 | 1.00 | 25.06 | H |
| ATOM | 3259 | CD | LYS | H | 222 | 29.405 | 19.108 | 23.650 | 1.00 | 27.85 | H |
| ATOM | 3260 | CE | LYS | H | 222 | 30.201 | 18.523 | 24.806 | 1.00 | 30.93 | H |
| ATOM | 3261 | NZ | LYS | H | 222 | 30.694 | 19.572 | 25.751 | 1.00 | 34.53 | H |
| ATOM | 3262 | C | LYS | H | 222 | 26.365 | 16.596 | 21.664 | 1.00 | 17.81 | H |
| ATOM | 3263 | O | LYS | H | 222 | 25.300 | 16.993 | 22.125 | 1.00 | 17.31 | H |
| ATOM | 3264 | N | VAL | H | 225 | 26.819 | 15.360 | 21.846 | 1.00 | 16.73 | H |
| ATOM | 3265 | CA | VAL | H | 225 | 26.080 | 14.382 | 22.637 | 1.00 | 18.39 | H |
| ATOM | 3266 | CB | VAL | H | 225 | 25.868 | 13.070 | 21.836 | 1.00 | 16.06 | H |
| ATOM | 3267 | CG1 | VAL | H | 225 | 25.003 | 12.109 | 22.626 | 1.00 | 17.56 | H |
| ATOM | 3268 | CG2 | VAL | H | 225 | 25.243 | 13.383 | 20.482 | 1.00 | 14.55 | H |
| ATOM | 3269 | C | VAL | H | 225 | 26.835 | 14.069 | 23.927 | 1.00 | 18.03 | H |
| ATOM | 3270 | O | VAL | H | 225 | 27.960 | 13.578 | 23.897 | 1.00 | 17.45 | H |
| ATOM | 3271 | N | GLU | H | 226 | 26.210 | 14.351 | 25.061 | 1.00 | 20.87 | H |
| ATOM | 3272 | CA | GLU | H | 226 | 26.849 | 14.098 | 26.345 | 1.00 | 27.69 | H |
| ATOM | 3273 | CB | GLU | H | 226 | 27.431 | 15.405 | 26.897 | 1.00 | 29.80 | H |
| ATOM | 3274 | CG | GLU | H | 226 | 26.429 | 16.534 | 26.985 | 1.00 | 34.68 | H |
| ATOM | 3275 | CD | GLU | H | 226 | 27.084 | 17.895 | 27.159 | 1.00 | 37.56 | H |
| ATOM | 3276 | OE1 | GLU | H | 226 | 26.350 | 18.906 | 27.149 | 1.00 | 41.46 | H |
| ATOM | 3277 | OE2 | GLU | H | 226 | 28.324 | 17.962 | 27.302 | 1.00 | 38.68 | H |
| ATOM | 3278 | C | GLU | H | 226 | 25.906 | 13.474 | 27.362 | 1.00 | 28.78 | H |
| ATOM | 3279 | O | GLU | H | 226 | 24.694 | 13.415 | 27.154 | 1.00 | 28.70 | H |
| ATOM | 3280 | N | PRO | H | 227 | 26.459 | 12.974 | 28.474 | 1.00 | 31.31 | H |
| ATOM | 3281 | CD | PRO | H | 227 | 27.891 | 12.812 | 28.782 | 1.00 | 33.28 | H |
| ATOM | 3282 | CA | PRO | H | 227 | 25.624 | 12.362 | 29.509 | 1.00 | 32.95 | H |
| ATOM | 3283 | CB | PRO | H | 227 | 26.641 | 11.947 | 30.566 | 1.00 | 33.03 | H |
| ATOM | 3284 | CG | PRO | H | 227 | 27.881 | 11.661 | 29.749 | 1.00 | 33.72 | H |
| ATOM | 3285 | C | PRO | H | 227 | 24.644 | 13.404 | 30.034 | 1.00 | 34.83 | H |
| ATOM | 3286 | O | PRO | H | 227 | 24.984 | 14.578 | 30.137 | 1.00 | 35.76 | H |
| ATOM | 3287 | N | LYS | H | 228 | 23.424 | 12.987 | 30.346 | 1.00 | 37.41 | H |
| ATOM | 3288 | CA | LYS | H | 228 | 22.449 | 13.931 | 30.870 | 1.00 | 39.94 | H |
| ATOM | 3289 | CB | LYS | H | 228 | 21.030 | 13.465 | 30.552 | 1.00 | 42.00 | H |
| ATOM | 3290 | CG | LYS | H | 228 | 19.975 | 14.526 | 30.803 | 1.00 | 44.82 | H |
| ATOM | 3291 | CD | LYS | H | 228 | 18.625 | 13.891 | 31.075 | 1.00 | 48.07 | H |
| ATOM | 3292 | CE | LYS | H | 228 | 17.547 | 14.425 | 30.146 | 1.00 | 50.08 | H |
| ATOM | 3293 | NZ | LYS | H | 228 | 16.202 | 13.915 | 30.540 | 1.00 | 51.00 | H |
| ATOM | 3294 | C | LYS | H | 228 | 22.631 | 14.022 | 32.386 | 1.00 | 40.24 | H |
| ATOM | 3295 | O | LYS | H | 228 | 22.911 | 12.975 | 33.005 | 1.00 | 39.97 | H |
| ATOM | 3296 | OXT | LYS | H | 228 | 22.480 | 15.130 | 32.943 | 1.00 | 41.98 | H |
| ATOM | 1 | CB | ASP | L | 1 | 5.212 | −13.750 | −17.671 | 1.00 | 41.03 | L |
| ATOM | 2 | CG | ASP | L | 1 | 5.931 | −12.900 | −16.634 | 1.00 | 46.26 | L |
| ATOM | 3 | OD1 | ASP | L | 1 | 5.488 | −12.876 | −15.464 | 1.00 | 49.24 | L |
| ATOM | 4 | OD2 | ASP | L | 1 | 6.929 | −12.241 | −16.989 | 1.00 | 49.12 | L |
| ATOM | 5 | C | ASP | L | 1 | 6.988 | −15.404 | −18.234 | 1.00 | 34.15 | L |
| ATOM | 6 | O | ASP | L | 1 | 6.481 | −16.302 | −17.562 | 1.00 | 33.49 | L |
| ATOM | 7 | N | ASP | L | 1 | 5.352 | −14.723 | −19.946 | 1.00 | 38.20 | L |
| ATOM | 8 | CA | ASP | L | 1 | 6.145 | −14.254 | −18.776 | 1.00 | 36.32 | L |
| ATOM | 9 | N | ILE | L | 2 | 8.277 | −15.385 | −18.542 | 1.00 | 30.13 | L |
| ATOM | 10 | CA | ILE | L | 2 | 9.166 | −16.418 | −18.045 | 1.00 | 26.76 | L |
| ATOM | 11 | CB | ILE | L | 2 | 10.411 | −16.544 | −18.927 | 1.00 | 25.22 | L |
| ATOM | 12 | CG2 | ILE | L | 2 | 11.428 | −17.478 | −18.273 | 1.00 | 24.52 | L |
| ATOM | 13 | CG1 | ILE | L | 2 | 10.001 | −17.060 | −20.306 | 1.00 | 26.23 | L |
| ATOM | 14 | CD1 | ILE | L | 2 | 11.142 | −17.143 | −21.293 | 1.00 | 26.57 | L |
| ATOM | 15 | C | ILE | L | 2 | 9.585 | −16.037 | −16.628 | 1.00 | 24.41 | L |
| ATOM | 16 | O | ILE | L | 2 | 10.154 | −14.965 | −16.410 | 1.00 | 24.64 | L |
| ATOM | 17 | N | VAL | L | 3 | 9.287 | −16.908 | −15.671 | 1.00 | 20.93 | L |
| ATOM | 18 | CA | VAL | L | 3 | 9.638 | −16.665 | −14.278 | 1.00 | 17.87 | L |
| ATOM | 19 | CB | VAL | L | 3 | 8.578 | −17.242 | −13.311 | 1.00 | 19.70 | L |
| ATOM | 20 | CG1 | VAL | L | 3 | 8.987 | −16.969 | −11.864 | 1.00 | 18.98 | L |
| ATOM | 21 | CG2 | VAL | L | 3 | 7.208 | −16.632 | −13.604 | 1.00 | 18.89 | L |
| ATOM | 22 | C | VAL | L | 3 | 10.989 | −17.303 | −13.944 | 1.00 | 19.31 | L |
| ATOM | 23 | O | VAL | L | 3 | 11.210 | −18.502 | −14.170 | 1.00 | 16.35 | L |
| ATOM | 24 | N | LEU | L | 4 | 11.898 | −16.488 | −13.423 | 1.00 | 16.87 | L |
| ATOM | 25 | CA | LEU | L | 4 | 13.213 | −16.968 | −13.043 | 1.00 | 16.92 | L |
| ATOM | 26 | CB | LEU | L | 4 | 14.297 | −15.996 | −13.517 | 1.00 | 15.72 | L |
| ATOM | 27 | CG | LEU | L | 4 | 14.399 | −15.829 | −15.035 | 1.00 | 14.94 | L |
| ATOM | 28 | CD1 | LEU | L | 4 | 15.541 | −14.877 | −15.355 | 1.00 | 15.70 | L |
| ATOM | 29 | CD2 | LEU | L | 4 | 14.622 | −17.175 | −15.709 | 1.00 | 15.05 | L |
| ATOM | 30 | C | LEU | L | 4 | 13.230 | −17.091 | −11.527 | 1.00 | 17.12 | L |
| ATOM | 31 | O | LEU | L | 4 | 12.940 | −16.133 | −10.811 | 1.00 | 17.23 | L |
| ATOM | 32 | N | THR | L | 5 | 13.554 | −18.282 | −11.044 | 1.00 | 16.20 | L |
| ATOM | 33 | CA | THR | L | 5 | 13.590 | −18.532 | −9.611 | 1.00 | 16.42 | L |
| ATOM | 34 | CB | THR | L | 5 | 12.750 | −19.787 | −9.241 | 1.00 | 18.26 | L |
| ATOM | 35 | OG1 | THR | L | 5 | 11.381 | −19.557 | −9.584 | 1.00 | 20.40 | L |
| ATOM | 36 | CG2 | THR | L | 5 | 12.835 | −20.080 | −7.750 | 1.00 | 18.02 | L |
| ATOM | 37 | C | THR | L | 5 | 15.016 | −18.725 | −9.126 | 1.00 | 15.42 | L |
| ATOM | 38 | O | THR | L | 5 | 15.753 | −19.581 | −9.623 | 1.00 | 13.82 | L |
| ATOM | 39 | N | GLN | L | 6 | 15.399 | −17.897 | −8.165 | 1.00 | 14.56 | L |
| ATOM | 40 | CA | GLN | L | 6 | 16.723 | −17.955 | −7.572 | 1.00 | 16.80 | L |
| ATOM | 41 | CB | GLN | L | 6 | 17.383 | −16.573 | −7.604 | 1.00 | 15.83 | L |
| ATOM | 42 | CG | GLN | L | 6 | 18.036 | −16.264 | −8.940 | 1.00 | 14.63 | L |
| ATOM | 43 | CD | GLN | L | 6 | 18.729 | −14.914 | −8.955 | 1.00 | 16.96 | L |
| ATOM | 44 | OE1 | GLN | L | 6 | 18.105 | −13.879 | −9.215 | 1.00 | 13.67 | L |
| ATOM | 45 | NE2 | GLN | L | 6 | 20.029 | −14.916 | −8.661 | 1.00 | 13.17 | L |
| ATOM | 46 | C | GLN | L | 6 | 16.534 | −18.436 | −6.146 | 1.00 | 16.89 | L |
| ATOM | 47 | O | GLN | L | 6 | 16.023 | −17.715 | −5.293 | 1.00 | 17.57 | L |
| ATOM | 48 | N | SER | L | 7 | 16.932 | −19.679 | −5.905 | 1.00 | 20.16 | L |
| ATOM | 49 | CA | SER | L | 7 | 16.768 | −20.293 | −4.596 | 1.00 | 20.66 | L |
| ATOM | 50 | CB | SER | L | 7 | 15.549 | −21.212 | −4.618 | 1.00 | 22.88 | L |
| ATOM | 51 | OG | SER | L | 7 | 14.624 | −20.849 | −3.609 | 1.00 | 32.94 | L |
| ATOM | 52 | C | SER | L | 7 | 17.998 | −21.091 | −4.197 | 1.00 | 18.88 | L |
| ATOM | 53 | O | SER | L | 7 | 18.543 | −21.846 | −5.000 | 1.00 | 19.73 | L |
| ATOM | 54 | N | PRO | L | 8 | 18.466 | −20.915 | −2.952 | 1.00 | 17.16 | L |
| ATOM | 55 | CD | PRO | L | 8 | 19.620 | −21.642 | −2.395 | 1.00 | 16.35 | L |
| ATOM | 56 | CA | PRO | L | 8 | 17.886 | −20.014 | −1.952 | 1.00 | 15.00 | L |
| ATOM | 57 | CB | PRO | L | 8 | 18.520 | −20.498 | −0.650 | 1.00 | 13.51 | L |
| ATOM | 58 | CG | PRO | L | 8 | 19.886 | −20.894 | −1.100 | 1.00 | 15.91 | L |
| ATOM | 59 | C | PRO | L | 8 | 18.189 | −18.542 | −2.237 | 1.00 | 14.74 | L |
| ATOM | 60 | O | PRO | L | 8 | 19.097 | −18.221 | −3.010 | 1.00 | 15.27 | L |
| ATOM | 61 | N | GLY | L | 9 | 17.423 | −17.661 | −1.602 | 1.00 | 14.82 | L |
| ATOM | 62 | CA | GLY | L | 9 | 17.594 | −16.227 | −1.776 | 1.00 | 15.76 | L |
| ATOM | 63 | C | GLY | L | 9 | 18.775 | −15.662 | −1.004 | 1.00 | 16.34 | L |
| ATOM | 64 | O | GLY | L | 9 | 19.198 | −14.523 | −1.242 | 1.00 | 14.98 | L |
| ATOM | 65 | N | THR | L | 10 | 19.292 | −16.448 | −0.063 | 1.00 | 14.91 | L |
| ATOM | 66 | CA | THR | L | 10 | 20.450 | −16.045 | 0.718 | 1.00 | 16.22 | L |
| ATOM | 67 | CB | THR | L | 10 | 20.062 | −15.468 | 2.108 | 1.00 | 18.79 | L |
| ATOM | 68 | OG1 | THR | L | 10 | 19.133 | −14.389 | 1.956 | 1.00 | 17.32 | L |
| ATOM | 69 | CG2 | THR | L | 10 | 21.308 | −14.946 | 2.818 | 1.00 | 17.59 | L |
| ATOM | 70 | C | THR | L | 10 | 21.388 | −17.226 | 0.971 | 1.00 | 15.81 | L |
| ATOM | 71 | O | THR | L | 10 | 20.945 | −18.331 | 1.282 | 1.00 | 18.36 | L |
| ATOM | 72 | N | MET | L | 11 | 22.685 | −16.986 | 0.813 | 1.00 | 13.91 | L |
| ATOM | 73 | CA | MET | L | 11 | 23.686 | −18.002 | 1.088 | 1.00 | 14.80 | L |
| ATOM | 74 | CB | MET | L | 11 | 24.384 | −18.484 | −0.187 | 1.00 | 14.41 | L |
| ATOM | 75 | CG | MET | L | 11 | 23.601 | −19.502 | −0.957 | 1.00 | 15.84 | L |
| ATOM | 76 | SD | MET | L | 11 | 24.663 | −20.632 | −1.873 | 1.00 | 26.26 | L |
| ATOM | 77 | CE | MET | L | 11 | 23.927 | −20.424 | −3.485 | 1.00 | 11.06 | L |
| ATOM | 78 | C | MET | L | 11 | 24.731 | −17.422 | 2.024 | 1.00 | 12.86 | L |
| ATOM | 79 | O | MET | L | 11 | 25.397 | −16.444 | 1.682 | 1.00 | 9.87 | L |
| ATOM | 80 | N | SER | L | 12 | 24.854 | −18.023 | 3.204 | 1.00 | 12.74 | L |
| ATOM | 81 | CA | SER | L | 12 | 25.836 | −17.615 | 4.201 | 1.00 | 14.28 | L |
| ATOM | 82 | CB | SER | L | 12 | 25.270 | −17.808 | 5.604 | 1.00 | 11.72 | L |
| ATOM | 83 | OG | SER | L | 12 | 24.108 | −17.021 | 5.783 | 1.00 | 14.42 | L |
| ATOM | 84 | C | SER | L | 12 | 27.055 | −18.517 | 4.002 | 1.00 | 15.60 | L |
| ATOM | 85 | O | SER | L | 12 | 27.009 | −19.709 | 4.324 | 1.00 | 17.38 | L |
| ATOM | 86 | N | LEU | L | 13 | 28.135 | −17.947 | 3.473 | 1.00 | 16.05 | L |
| ATOM | 87 | CA | LEU | L | 13 | 29.356 | −18.701 | 3.193 | 1.00 | 17.74 | L |
| ATOM | 88 | CB | LEU | L | 13 | 29.509 | −18.880 | 1.678 | 1.00 | 14.89 | L |
| ATOM | 89 | CG | LEU | L | 13 | 28.420 | −19.662 | 0.935 | 1.00 | 13.78 | L |
| ATOM | 90 | CD1 | LEU | L | 13 | 28.551 | −19.430 | −0.560 | 1.00 | 14.03 | L |
| ATOM | 91 | CD2 | LEU | L | 13 | 28.538 | −21.153 | 1.261 | 1.00 | 15.44 | L |
| ATOM | 92 | C | LEU | L | 13 | 30.621 | −18.038 | 3.752 | 1.00 | 19.60 | L |
| ATOM | 93 | O | LEU | L | 13 | 30.638 | −16.838 | 4.017 | 1.00 | 17.50 | L |
| ATOM | 94 | N | SER | L | 14 | 31.683 | −18.826 | 3.904 | 1.00 | 19.70 | L |
| ATOM | 95 | CA | SER | L | 14 | 32.947 | −18.320 | 4.438 | 1.00 | 20.46 | L |
| ATOM | 96 | CB | SER | L | 14 | 33.693 | −19.430 | 5.191 | 1.00 | 21.80 | L |
| ATOM | 97 | OG | SER | L | 14 | 32.906 | −19.967 | 6.242 | 1.00 | 22.40 | L |
| ATOM | 98 | C | SER | L | 14 | 33.867 | −17.758 | 3.364 | 1.00 | 20.61 | L |
| ATOM | 99 | O | SER | L | 14 | 33.853 | −18.205 | 2.218 | 1.00 | 18.72 | L |
| ATOM | 100 | N | PRO | L | 15 | 34.687 | −16.759 | 3.729 | 1.00 | 20.86 | L |
| ATOM | 101 | CD | PRO | L | 15 | 34.822 | −16.141 | 5.059 | 1.00 | 21.75 | L |
| ATOM | 102 | CA | PRO | L | 15 | 35.617 | −16.154 | 2.774 | 1.00 | 20.65 | L |
| ATOM | 103 | CB | PRO | L | 15 | 36.444 | −15.214 | 3.653 | 1.00 | 21.53 | L |
| ATOM | 104 | CG | PRO | L | 15 | 35.484 | −14.823 | 4.730 | 1.00 | 20.20 | L |
| ATOM | 105 | C | PRO | L | 15 | 36.466 | −17.267 | 2.168 | 1.00 | 19.31 | L |
| ATOM | 106 | O | PRO | L | 15 | 36.908 | −18.161 | 2.881 | 1.00 | 17.07 | L |
| ATOM | 107 | N | GLY | L | 16 | 36.677 | −17.224 | 0.858 | 1.00 | 20.72 | L |
| ATOM | 108 | CA | GLY | L | 16 | 37.475 | −18.250 | 0.211 | 1.00 | 19.04 | L |
| ATOM | 109 | C | GLY | L | 16 | 36.700 | −19.505 | −0.157 | 1.00 | 21.11 | L |
| ATOM | 110 | O | GLY | L | 16 | 37.210 | −20.363 | −0.872 | 1.00 | 22.57 | L |
| ATOM | 111 | N | GLU | L | 17 | 35.470 | −19.627 | 0.328 | 1.00 | 20.30 | L |
| ATOM | 112 | CA | GLU | L | 17 | 34.664 | −20.794 | 0.013 | 1.00 | 22.02 | L |
| ATOM | 113 | CB | GLU | L | 17 | 33.459 | −20.894 | 0.951 | 1.00 | 25.05 | L |
| ATOM | 114 | CG | GLU | L | 17 | 33.508 | −22.047 | 1.926 | 1.00 | 32.27 | L |
| ATOM | 115 | CD | GLU | L | 17 | 32.128 | −22.406 | 2.455 | 1.00 | 36.73 | L |
| ATOM | 116 | OE1 | GLU | L | 17 | 31.517 | −21.573 | 3.160 | 1.00 | 36.03 | L |
| ATOM | 117 | OE2 | GLU | L | 17 | 31.651 | −23.525 | 2.157 | 1.00 | 37.56 | L |
| ATOM | 118 | C | GLU | L | 17 | 34.155 | −20.740 | −1.423 | 1.00 | 21.07 | L |
| ATOM | 119 | O | GLU | L | 17 | 34.110 | −19.683 | −2.048 | 1.00 | 19.64 | L |
| ATOM | 120 | N | ARG | L | 18 | 33.778 | −21.898 | −1.943 | 1.00 | 20.65 | L |
| ATOM | 121 | CA | ARG | L | 18 | 33.238 | −21.976 | −3.290 | 1.00 | 22.24 | L |
| ATOM | 122 | CB | ARG | L | 18 | 33.429 | −23.393 | −3.836 | 1.00 | 23.51 | L |
| ATOM | 123 | CG | ARG | L | 18 | 32.948 | −23.634 | −5.262 | 1.00 | 27.00 | L |
| ATOM | 124 | CD | ARG | L | 18 | 33.378 | −25.030 | −5.714 | 1.00 | 31.33 | L |
| ATOM | 125 | NE | ARG | L | 18 | 33.219 | −25.236 | −7.150 | 1.00 | 36.96 | L |
| ATOM | 126 | CZ | ARG | L | 18 | 32.142 | −25.772 | −7.715 | 1.00 | 38.37 | L |
| ATOM | 127 | NH1 | ARG | L | 18 | 32.086 | −25.914 | −9.032 | 1.00 | 40.55 | L |
| ATOM | 128 | NH2 | ARG | L | 18 | 31.128 | −26.180 | −6.962 | 1.00 | 38.93 | L |
| ATOM | 129 | C | ARG | L | 18 | 31.746 | −21.644 | −3.190 | 1.00 | 20.01 | L |
| ATOM | 130 | O | ARG | L | 18 | 31.151 | −21.748 | −2.118 | 1.00 | 17.95 | L |
| ATOM | 131 | N | VAL | L | 19 | 31.144 | −21.226 | −4.295 | 1.00 | 20.16 | L |
| ATOM | 132 | CA | VAL | L | 19 | 29.718 | −20.935 | −4.271 | 1.00 | 19.58 | L |
| ATOM | 133 | CB | VAL | L | 19 | 29.436 | −19.485 | −3.799 | 1.00 | 21.11 | L |
| ATOM | 134 | CG1 | VAL | L | 19 | 30.113 | −18.505 | −4.693 | 1.00 | 25.30 | L |
| ATOM | 135 | CG2 | VAL | L | 19 | 27.942 | −19.225 | −3.773 | 1.00 | 29.25 | L |
| ATOM | 136 | C | VAL | L | 19 | 29.031 | −21.167 | −5.607 | 1.00 | 15.44 | L |
| ATOM | 137 | O | VAL | L | 19 | 29.541 | −20.787 | −6.661 | 1.00 | 14.08 | L |
| ATOM | 138 | N | THR | L | 20 | 27.868 | −21.805 | −5.543 | 1.00 | 14.54 | L |
| ATOM | 139 | CA | THR | L | 20 | 27.080 | −22.078 | −6.734 | 1.00 | 15.10 | L |
| ATOM | 140 | CB | THR | L | 20 | 27.018 | −23.602 | −7.042 | 1.00 | 15.02 | L |
| ATOM | 141 | OG1 | THR | L | 20 | 26.479 | −24.302 | −5.917 | 1.00 | 17.05 | L |
| ATOM | 142 | CG2 | THR | L | 20 | 28.418 | −24.140 | −7.345 | 1.00 | 15.74 | L |
| ATOM | 143 | C | THR | L | 20 | 25.668 | −21.530 | −6.523 | 1.00 | 14.99 | L |
| ATOM | 144 | O | THR | L | 20 | 24.993 | −21.860 | −5.542 | 1.00 | 12.67 | L |
| ATOM | 145 | N | LEU | L | 21 | 25.248 | −20.665 | −7.437 | 1.00 | 13.77 | L |
| ATOM | 146 | CA | LEU | L | 21 | 23.928 | −20.046 | −7.385 | 1.00 | 13.22 | L |
| ATOM | 147 | CB | LEU | L | 21 | 24.037 | −18.523 | −7.538 | 1.00 | 12.60 | L |
| ATOM | 148 | CG | LEU | L | 21 | 24.382 | −17.707 | −6.284 | 1.00 | 14.05 | L |
| ATOM | 149 | CD1 | LEU | L | 21 | 25.739 | −18.117 | −5.750 | 1.00 | 13.64 | L |
| ATOM | 150 | CD2 | LEU | L | 21 | 24.369 | −16.231 | −6.622 | 1.00 | 13.66 | L |
| ATOM | 151 | C | LEU | L | 21 | 23.051 | −20.603 | −8.502 | 1.00 | 12.48 | L |
| ATOM | 152 | O | LEU | L | 21 | 23.451 | −20.648 | −9.664 | 1.00 | 11.50 | L |
| ATOM | 153 | N | SER | L | 22 | 21.850 | −21.015 | −8.129 | 1.00 | 13.00 | L |
| ATOM | 154 | CA | SER | L | 22 | 20.888 | −21.580 | −9.058 | 1.00 | 13.50 | L |
| ATOM | 155 | CB | SER | L | 22 | 20.149 | −22.726 | −8.360 | 1.00 | 14.47 | L |
| ATOM | 156 | OG | SER | L | 22 | 18.968 | −23.085 | −9.056 | 1.00 | 19.18 | L |
| ATOM | 157 | C | SER | L | 22 | 19.857 | −20.572 | −9.583 | 1.00 | 12.59 | L |
| ATOM | 158 | O | SER | L | 22 | 19.354 | −19.736 | −8.835 | 1.00 | 11.40 | L |
| ATOM | 159 | N | CYS | L | 23 | 19.547 | −20.671 | −10.871 | 1.00 | 14.35 | L |
| ATOM | 160 | CA | CYS | L | 23 | 18.531 | −19.833 | −11.509 | 1.00 | 15.92 | L |
| ATOM | 161 | C | CYS | L | 23 | 17.728 | −20.782 | −12.395 | 1.00 | 14.90 | L |
| ATOM | 162 | O | CYS | L | 23 | 18.249 | −21.319 | −13.369 | 1.00 | 15.82 | L |
| ATOM | 163 | CB | CYS | L | 23 | 19.167 | −18.729 | −12.363 | 1.00 | 14.71 | L |
| ATOM | 164 | SG | CYS | L | 23 | 18.024 | −17.664 | −13.333 | 1.00 | 19.52 | L |
| ATOM | 165 | N | ARG | L | 24 | 16.468 | −21.002 | −12.035 | 1.00 | 17.94 | L |
| ATOM | 166 | CA | ARG | L | 24 | 15.584 | −21.905 | −12.784 | 1.00 | 20.68 | L |
| ATOM | 167 | CB | ARG | L | 24 | 14.943 | −22.914 | −11.838 | 1.00 | 23.64 | L |
| ATOM | 168 | CG | ARG | L | 24 | 15.919 | −23.770 | −11.091 | 1.00 | 31.30 | L |
| ATOM | 169 | CD | ARG | L | 24 | 15.895 | −25.185 | −11.604 | 1.00 | 35.41 | L |
| ATOM | 170 | NE | ARG | L | 24 | 16.419 | −26.087 | −10.588 | 1.00 | 40.63 | L |
| ATOM | 171 | CZ | ARG | L | 24 | 16.408 | −27.412 | −10.671 | 1.00 | 41.04 | L |
| ATOM | 172 | NH1 | ARG | L | 24 | 15.896 | −28.021 | −11.735 | 1.00 | 41.13 | L |
| ATOM | 173 | NH2 | ARG | L | 24 | 16.915 | −28.127 | −9.679 | 1.00 | 40.81 | L |
| ATOM | 174 | C | ARG | L | 24 | 14.475 | −21.137 | −13.485 | 1.00 | 17.77 | L |
| ATOM | 175 | O | ARG | L | 24 | 13.815 | −20.306 | −12.866 | 1.00 | 18.62 | L |
| ATOM | 176 | N | ALA | L | 25 | 14.260 | −21.434 | −14.762 | 1.00 | 17.55 | L |
| ATOM | 177 | CA | ALA | L | 25 | 13.234 | −20.759 | −15.552 | 1.00 | 17.27 | L |
| ATOM | 178 | CB | ALA | L | 25 | 13.768 | −20.472 | −16.934 | 1.00 | 14.55 | L |
| ATOM | 179 | C | ALA | L | 25 | 11.947 | −21.569 | −15.656 | 1.00 | 18.29 | L |
| ATOM | 180 | O | ALA | L | 25 | 11.983 | −22.793 | −15.738 | 1.00 | 16.41 | L |
| ATOM | 181 | N | SER | L | 26 | 10.811 | −20.878 | −15.665 | 1.00 | 20.27 | L |
| ATOM | 182 | CA | SER | L | 26 | 9.516 | −21.540 | −15.768 | 1.00 | 22.31 | L |
| ATOM | 183 | CB | SER | L | 26 | 8.388 | −20.529 | −15.554 | 1.00 | 22.03 | L |
| ATOM | 184 | OG | SER | L | 26 | 8.600 | −19.348 | −16.306 | 1.00 | 23.03 | L |
| ATOM | 185 | C | SER | L | 26 | 9.356 | −22.242 | −17.117 | 1.00 | 25.18 | L |
| ATOM | 186 | O | SER | L | 26 | 8.488 | −23.095 | −17.282 | 1.00 | 27.63 | L |
| ATOM | 187 | N | GLN | L | 27 | 10.200 | −21.878 | −18.077 | 1.00 | 25.92 | L |
| ATOM | 188 | CA | GLN | L | 27 | 10.185 | −22.486 | −19.403 | 1.00 | 27.06 | L |
| ATOM | 189 | CB | GLN | L | 27 | 9.051 | −21.912 | −20.259 | 1.00 | 29.62 | L |
| ATOM | 190 | CG | GLN | L | 27 | 9.082 | −20.409 | −20.400 | 1.00 | 34.79 | L |
| ATOM | 191 | CD | GLN | L | 27 | 7.947 | −19.881 | −21.254 | 1.00 | 37.08 | L |
| ATOM | 192 | OE1 | GLN | L | 27 | 7.891 | −20.132 | −22.457 | 1.00 | 39.54 | L |
| ATOM | 193 | NE2 | GLN | L | 27 | 7.032 | −19.148 | −20.633 | 1.00 | 35.79 | L |
| ATOM | 194 | C | GLN | L | 27 | 11.530 | −22.236 | −20.075 | 1.00 | 26.43 | L |
| ATOM | 195 | O | GLN | L | 27 | 12.311 | −21.398 | −19.626 | 1.00 | 25.72 | L |
| ATOM | 196 | N | SER | L | 27A | 11.798 | −22.976 | −21.144 | 1.00 | 25.04 | L |
| ATOM | 197 | CA | SER | L | 27A | 13.056 | −22.867 | −21.870 | 1.00 | 26.10 | L |
| ATOM | 198 | CB | SER | L | 27A | 12.977 | −23.697 | −23.155 | 1.00 | 27.29 | L |
| ATOM | 199 | OG | SER | L | 27A | 14.109 | −23.471 | −23.974 | 1.00 | 34.20 | L |
| ATOM | 200 | C | SER | L | 27A | 13.449 | −21.431 | −22.209 | 1.00 | 24.83 | L |
| ATOM | 201 | O | SER | L | 27A | 12.625 | −20.647 | −22.684 | 1.00 | 24.55 | L |
| ATOM | 202 | N | VAL | L | 28 | 14.712 | −21.092 | −21.960 | 1.00 | 22.42 | L |
| ATOM | 203 | CA | VAL | L | 28 | 15.219 | −19.757 | −22.257 | 1.00 | 20.59 | L |
| ATOM | 204 | CB | VAL | L | 28 | 16.319 | −19.339 | −21.253 | 1.00 | 18.28 | L |
| ATOM | 205 | CG1 | VAL | L | 28 | 16.913 | −18.010 | −21.660 | 1.00 | 17.18 | L |
| ATOM | 206 | CG2 | VAL | L | 28 | 15.739 | −19.252 | −19.853 | 1.00 | 19.29 | L |
| ATOM | 207 | C | VAL | L | 28 | 15.787 | −19.720 | −23.679 | 1.00 | 19.57 | L |
| ATOM | 208 | O | VAL | L | 28 | 16.764 | −20.398 | −23.989 | 1.00 | 18.94 | L |
| ATOM | 209 | N | GLY | L | 29 | 15.165 | −18.916 | −24.535 | 1.00 | 20.72 | L |
| ATOM | 210 | CA | GLY | L | 29 | 15.596 | −18.801 | −25.920 | 1.00 | 19.27 | L |
| ATOM | 211 | C | GLY | L | 29 | 17.073 | −18.548 | −26.171 | 1.00 | 19.98 | L |
| ATOM | 212 | O | GLY | L | 29 | 17.640 | −17.554 | −25.708 | 1.00 | 18.84 | L |
| ATOM | 213 | N | SER | L | 30 | 17.694 | −19.453 | −26.925 | 1.00 | 20.48 | L |
| ATOM | 214 | CA | SER | L | 30 | 19.104 | −19.350 | −27.272 | 1.00 | 21.60 | L |
| ATOM | 215 | CB | SER | L | 30 | 19.312 | −18.209 | −28.274 | 1.00 | 23.97 | L |
| ATOM | 216 | OG | SER | L | 30 | 18.665 | −18.493 | −29.512 | 1.00 | 29.26 | L |
| ATOM | 217 | C | SER | L | 30 | 20.011 | −19.146 | −26.063 | 1.00 | 21.54 | L |
| ATOM | 218 | O | SER | L | 30 | 21.042 | −18.484 | −26.157 | 1.00 | 20.59 | L |
| ATOM | 219 | N | ASN | L | 31 | 19.620 | −19.721 | −24.930 | 1.00 | 20.34 | L |
| ATOM | 220 | CA | ASN | L | 31 | 20.400 | −19.612 | −23.703 | 1.00 | 22.68 | L |
| ATOM | 221 | CB | ASN | L | 31 | 21.649 | −20.483 | −23.815 | 1.00 | 24.35 | L |
| ATOM | 222 | CG | ASN | L | 31 | 21.316 | −21.952 | −23.847 | 1.00 | 25.69 | L |
| ATOM | 223 | OD1 | ASN | L | 31 | 22.086 | −22.762 | −24.354 | 1.00 | 29.34 | L |
| ATOM | 224 | ND2 | ASN | L | 31 | 20.157 | −22.308 | −23.293 | 1.00 | 25.90 | L |
| ATOM | 225 | C | ASN | L | 31 | 20.797 | −18.183 | −23.362 | 1.00 | 20.26 | L |
| ATOM | 226 | O | ASN | L | 31 | 21.895 | −17.932 | −22.862 | 1.00 | 20.04 | L |
| ATOM | 227 | N | PHE | L | 32 | 19.901 | −17.247 | −23.639 | 1.00 | 19.54 | L |
| ATOM | 228 | CA | PHE | L | 32 | 20.168 | −15.848 | −23.342 | 1.00 | 19.25 | L |
| ATOM | 229 | CB | PHE | L | 32 | 19.290 | −14.954 | −24.217 | 1.00 | 20.41 | L |
| ATOM | 230 | CG | PHE | L | 32 | 19.845 | −14.728 | −25.600 | 1.00 | 23.09 | L |
| ATOM | 231 | CD1 | PHE | L | 32 | 19.077 | −14.105 | −26.577 | 1.00 | 28.35 | L |
| ATOM | 232 | CD2 | PHE | L | 32 | 21.154 | −15.081 | −25.912 | 1.00 | 25.82 | L |
| ATOM | 233 | CE1 | PHE | L | 32 | 19.603 | −13.834 | −27.842 | 1.00 | 28.67 | L |
| ATOM | 234 | CE2 | PHE | L | 32 | 21.690 | −14.813 | −27.176 | 1.00 | 28.48 | L |
| ATOM | 235 | CZ | PHE | L | 32 | 20.909 | −14.186 | −28.142 | 1.00 | 26.27 | L |
| ATOM | 236 | C | PHE | L | 32 | 19.927 | −15.578 | −21.859 | 1.00 | 18.40 | L |
| ATOM | 237 | O | PHE | L | 32 | 18.918 | −14.983 | −21.464 | 1.00 | 17.43 | L |
| ATOM | 238 | N | LEU | L | 33 | 20.854 | −16.057 | −21.037 | 1.00 | 16.97 | L |
| ATOM | 239 | CA | LEU | L | 33 | 20.754 | −15.868 | −19.599 | 1.00 | 16.90 | L |
| ATOM | 240 | CB | LEU | L | 33 | 20.735 | −17.199 | −18.848 | 1.00 | 16.43 | L |
| ATOM | 241 | CG | LEU | L | 33 | 20.316 | −16.855 | −17.415 | 1.00 | 19.87 | L |
| ATOM | 242 | CD1 | LEU | L | 33 | 18.855 | −17.225 | −17.231 | 1.00 | 18.38 | L |
| ATOM | 243 | CD2 | LEU | L | 33 | 21.204 | −17.539 | −16.405 | 1.00 | 20.17 | L |
| ATOM | 244 | C | LEU | L | 33 | 21.964 | −15.079 | −19.165 | 1.00 | 14.91 | L |
| ATOM | 245 | O | LEU | L | 33 | 23.090 | −15.394 | −19.560 | 1.00 | 16.17 | L |
| ATOM | 246 | N | ALA | L | 34 | 21.734 | −14.058 | −18.352 | 1.00 | 12.74 | L |
| ATOM | 247 | CA | ALA | L | 34 | 22.821 | −13.211 | −17.888 | 1.00 | 11.45 | L |
| ATOM | 248 | CB | ALA | L | 34 | 22.680 | −11.820 | −18.486 | 1.00 | 8.89 | L |
| ATOM | 249 | C | ALA | L | 34 | 22.831 | −13.120 | −16.377 | 1.00 | 12.18 | L |
| ATOM | 250 | O | ALA | L | 34 | 21.790 | −13.259 | −15.739 | 1.00 | 12.33 | L |
| ATOM | 251 | N | TRP | L | 35 | 24.011 | −12.877 | −15.812 | 1.00 | 12.79 | L |
| ATOM | 252 | CA | TRP | L | 35 | 24.156 | −12.741 | −14.366 | 1.00 | 13.24 | L |
| ATOM | 253 | CB | TRP | L | 35 | 25.074 | −13.828 | −13.798 | 1.00 | 11.69 | L |
| ATOM | 254 | CG | TRP | L | 35 | 24.498 | −15.204 | −13.800 | 1.00 | 13.84 | L |
| ATOM | 255 | CD2 | TRP | L | 35 | 23.721 | −15.804 | −12.760 | 1.00 | 13.29 | L |
| ATOM | 256 | CE2 | TRP | L | 35 | 23.442 | −17.132 | −13.157 | 1.00 | 13.84 | L |
| ATOM | 257 | CE3 | TRP | L | 35 | 23.239 | −15.351 | −11.525 | 1.00 | 13.98 | L |
| ATOM | 258 | CD1 | TRP | L | 35 | 24.648 | −16.161 | −14.768 | 1.00 | 11.97 | L |
| ATOM | 259 | NE1 | TRP | L | 35 | 24.018 | −17.322 | −14.385 | 1.00 | 15.09 | L |
| ATOM | 260 | CZ2 | TRP | L | 35 | 22.702 | −18.014 | −12.361 | 1.00 | 13.77 | L |
| ATOM | 261 | CZ3 | TRP | L | 35 | 22.504 | −16.228 | −10.734 | 1.00 | 16.42 | L |
| ATOM | 262 | CH2 | TRP | L | 35 | 22.244 | −17.547 | −11.157 | 1.00 | 12.12 | L |
| ATOM | 263 | C | TRP | L | 35 | 24.755 | −11.385 | −14.021 | 1.00 | 13.46 | L |
| ATOM | 264 | O | TRP | L | 35 | 25.688 | −10.927 | −14.683 | 1.00 | 15.84 | L |
| ATOM | 265 | N | TYR | L | 36 | 24.231 | −10.749 | −12.981 | 1.00 | 12.97 | L |
| ATOM | 266 | CA | TYR | L | 36 | 24.754 | −9.458 | −12.555 | 1.00 | 13.18 | L |
| ATOM | 267 | CB | TYR | L | 36 | 23.763 | −8.318 | −12.824 | 1.00 | 13.50 | L |
| ATOM | 268 | CG | TYR | L | 36 | 23.308 | −8.185 | −14.254 | 1.00 | 15.77 | L |
| ATOM | 269 | CD1 | TYR | L | 36 | 22.297 | −9.004 | −14.757 | 1.00 | 14.62 | L |
| ATOM | 270 | CE1 | TYR | L | 36 | 21.861 | −8.885 | −16.063 | 1.00 | 12.11 | L |
| ATOM | 271 | CD2 | TYR | L | 36 | 23.881 | −7.239 | −15.108 | 1.00 | 11.00 | L |
| ATOM | 272 | CE2 | TYR | L | 36 | 23.454 | −7.117 | −16.431 | 1.00 | 12.74 | L |
| ATOM | 273 | CZ | TYR | L | 36 | 22.441 | −7.945 | −16.899 | 1.00 | 13.86 | L |
| ATOM | 274 | OH | TYR | L | 36 | 22.005 | −7.848 | −18.199 | 1.00 | 14.61 | L |
| ATOM | 275 | C | TYR | L | 36 | 25.072 | −9.441 | −11.072 | 1.00 | 13.31 | L |
| ATOM | 276 | O | TYR | L | 36 | 24.488 | −10.180 | −10.269 | 1.00 | 10.64 | L |
| ATOM | 277 | N | GLN | L | 37 | 26.007 | −8.577 | −10.710 | 1.00 | 14.62 | L |
| ATOM | 278 | CA | GLN | L | 37 | 26.360 | −8.423 | −9.318 | 1.00 | 13.81 | L |
| ATOM | 279 | CB | GLN | L | 37 | 27.858 | −8.619 | −9.115 | 1.00 | 15.04 | L |
| ATOM | 280 | CG | GLN | L | 37 | 28.314 | −8.388 | −7.683 | 1.00 | 14.70 | L |
| ATOM | 281 | CD | GLN | L | 37 | 29.827 | −8.438 | −7.544 | 1.00 | 20.18 | L |
| ATOM | 282 | OE1 | GLN | L | 37 | 30.405 | −9.473 | −7.183 | 1.00 | 20.33 | L |
| ATOM | 283 | NE2 | GLN | L | 37 | 30.481 | −7.322 | −7.850 | 1.00 | 12.85 | L |
| ATOM | 284 | C | GLN | L | 37 | 25.978 | −7.011 | −8.909 | 1.00 | 12.58 | L |
| ATOM | 285 | O | GLN | L | 37 | 26.116 | −6.068 | −9.698 | 1.00 | 12.43 | L |
| ATOM | 286 | N | GLN | L | 38 | 25.472 | −6.861 | −7.692 | 1.00 | 12.89 | L |
| ATOM | 287 | CA | GLN | L | 38 | 25.144 | −5.533 | −7.203 | 1.00 | 13.99 | L |
| ATOM | 288 | CB | GLN | L | 38 | 23.663 | −5.194 | −7.420 | 1.00 | 13.39 | L |
| ATOM | 289 | CG | GLN | L | 38 | 23.330 | −3.757 | −6.993 | 1.00 | 14.29 | L |
| ATOM | 290 | CD | GLN | L | 38 | 21.897 | −3.343 | −7.292 | 1.00 | 14.68 | L |
| ATOM | 291 | OE1 | GLN | L | 38 | 20.962 | −4.110 | −7.093 | 1.00 | 12.79 | L |
| ATOM | 292 | NE2 | GLN | L | 38 | 21.724 | −2.109 | −7.747 | 1.00 | 13.68 | L |
| ATOM | 293 | C | GLN | L | 38 | 25.504 | −5.372 | −5.729 | 1.00 | 15.01 | L |
| ATOM | 294 | O | GLN | L | 38 | 25.052 | −6.138 | −4.873 | 1.00 | 13.59 | L |
| ATOM | 295 | N | LYS | L | 39 | 26.346 | −4.381 | −5.452 | 1.00 | 15.46 | L |
| ATOM | 296 | CA | LYS | L | 39 | 26.776 | −4.076 | −4.092 | 1.00 | 18.51 | L |
| ATOM | 297 | CB | LYS | L | 39 | 28.244 | −3.642 | −4.067 | 1.00 | 19.15 | L |
| ATOM | 298 | CG | LYS | L | 39 | 29.226 | −4.670 | −4.624 | 1.00 | 24.65 | L |
| ATOM | 299 | CD | LYS | L | 39 | 30.660 | −4.295 | −4.256 | 1.00 | 27.38 | L |
| ATOM | 300 | CE | LYS | L | 39 | 31.661 | −5.336 | −4.729 | 1.00 | 27.00 | L |
| ATOM | 301 | NZ | LYS | L | 39 | 31.916 | −5.260 | −6.182 | 1.00 | 28.05 | L |
| ATOM | 302 | C | LYS | L | 39 | 25.893 | −2.927 | −3.631 | 1.00 | 18.25 | L |
| ATOM | 303 | O | LYS | L | 39 | 25.387 | −2.169 | −4.450 | 1.00 | 19.50 | L |
| ATOM | 304 | N | PRO | L | 40 | 25.695 | −2.783 | −2.313 | 1.00 | 20.68 | L |
| ATOM | 305 | CD | PRO | L | 40 | 26.270 | −3.590 | −1.223 | 1.00 | 20.69 | L |
| ATOM | 306 | CA | PRO | L | 40 | 24.854 | −1.707 | −1.775 | 1.00 | 20.24 | L |
| ATOM | 307 | CB | PRO | L | 40 | 25.056 | −1.832 | −0.268 | 1.00 | 20.56 | L |
| ATOM | 308 | CG | PRO | L | 40 | 25.309 | −3.313 | −0.091 | 1.00 | 21.40 | L |
| ATOM | 309 | C | PRO | L | 40 | 25.272 | −0.345 | −2.302 | 1.00 | 21.49 | L |
| ATOM | 310 | O | PRO | L | 40 | 26.463 | −0.047 | −2.397 | 1.00 | 21.41 | L |
| ATOM | 311 | N | GLY | L | 41 | 24.287 | 0.472 | −2.666 | 1.00 | 22.46 | L |
| ATOM | 312 | CA | GLY | L | 41 | 24.580 | 1.802 | −3.170 | 1.00 | 23.76 | L |
| ATOM | 313 | C | GLY | L | 41 | 25.174 | 1.873 | −4.568 | 1.00 | 24.55 | L |
| ATOM | 314 | O | GLY | L | 41 | 25.430 | 2.962 | −5.080 | 1.00 | 26.05 | L |
| ATOM | 315 | N | LYS | L | 42 | 25.399 | 0.727 | −5.199 | 1.00 | 22.96 | L |
| ATOM | 316 | CA | LYS | L | 42 | 25.965 | 0.736 | −6.540 | 1.00 | 21.53 | L |
| ATOM | 317 | CB | LYS | L | 42 | 27.284 | −0.050 | −6.574 | 1.00 | 20.29 | L |
| ATOM | 318 | CG | LYS | L | 42 | 28.374 | 0.571 | −5.721 | 1.00 | 22.76 | L |
| ATOM | 319 | CD | LYS | L | 42 | 29.764 | 0.155 | −6.178 | 1.00 | 28.47 | L |
| ATOM | 320 | CE | LYS | L | 42 | 30.294 | −1.007 | −5.368 | 1.00 | 29.04 | L |
| ATOM | 321 | NZ | LYS | L | 42 | 30.490 | −0.609 | −3.945 | 1.00 | 32.09 | L |
| ATOM | 322 | C | LYS | L | 42 | 25.013 | 0.182 | −7.588 | 1.00 | 19.96 | L |
| ATOM | 323 | O | LYS | L | 42 | 24.023 | −0.479 | −7.272 | 1.00 | 19.18 | L |
| ATOM | 324 | N | ALA | L | 43 | 25.321 | 0.477 | −8.844 | 1.00 | 18.00 | L |
| ATOM | 325 | CA | ALA | L | 43 | 24.531 | −0.001 | −9.960 | 1.00 | 17.72 | L |
| ATOM | 326 | CB | ALA | L | 43 | 24.783 | 0.878 | −11.183 | 1.00 | 16.01 | L |
| ATOM | 327 | C | ALA | L | 43 | 24.970 | −1.442 | −10.237 | 1.00 | 17.81 | L |
| ATOM | 328 | O | ALA | L | 43 | 26.093 | −1.825 | −9.909 | 1.00 | 17.44 | L |
| ATOM | 329 | N | PRO | L | 44 | 24.084 | −2.261 | −10.828 | 1.00 | 17.06 | L |
| ATOM | 330 | CD | PRO | L | 44 | 22.678 | −1.958 | −11.146 | 1.00 | 16.04 | L |
| ATOM | 331 | CA | PRO | L | 44 | 24.399 | −3.659 | −11.144 | 1.00 | 16.18 | L |
| ATOM | 332 | CB | PRO | L | 44 | 23.095 | −4.176 | −11.751 | 1.00 | 19.05 | L |
| ATOM | 333 | CG | PRO | L | 44 | 22.047 | −3.317 | −11.086 | 1.00 | 16.41 | L |
| ATOM | 334 | C | PRO | L | 44 | 25.561 | −3.745 | −12.131 | 1.00 | 16.70 | L |
| ATOM | 335 | O | PRO | L | 44 | 25.818 | −2.797 | −12.879 | 1.00 | 14.30 | L |
| ATOM | 336 | N | LYS | L | 45 | 26.267 | −4.875 | −12.127 | 1.00 | 16.97 | L |
| ATOM | 337 | CA | LYS | L | 45 | 27.387 | −5.060 | −13.041 | 1.00 | 15.95 | L |
| ATOM | 338 | CB | LYS | L | 45 | 28.716 | −4.930 | −12.297 | 1.00 | 19.48 | L |
| ATOM | 339 | CG | LYS | L | 45 | 29.904 | −4.817 | −13.248 | 1.00 | 21.24 | L |
| ATOM | 340 | CD | LYS | L | 45 | 31.242 | −4.824 | −12.530 | 1.00 | 25.41 | L |
| ATOM | 341 | CE | LYS | L | 45 | 31.811 | −6.227 | −12.444 | 1.00 | 29.13 | L |
| ATOM | 342 | NZ | LYS | L | 45 | 33.269 | −6.201 | −12.106 | 1.00 | 32.06 | L |
| ATOM | 343 | C | LYS | L | 45 | 27.318 | −6.417 | −13.744 | 1.00 | 14.57 | L |
| ATOM | 344 | O | LYS | L | 45 | 27.127 | −7.448 | −13.102 | 1.00 | 15.58 | L |
| ATOM | 345 | N | LEU | L | 46 | 27.477 | −6.411 | −15.063 | 1.00 | 11.75 | L |
| ATOM | 346 | CA | LEU | L | 46 | 27.411 | −7.632 | −15.854 | 1.00 | 11.69 | L |
| ATOM | 347 | CB | LEU | L | 46 | 27.352 | −7.297 | −17.348 | 1.00 | 8.39 | L |
| ATOM | 348 | CG | LEU | L | 46 | 27.221 | −8.483 | −18.316 | 1.00 | 10.14 | L |
| ATOM | 349 | CD1 | LEU | L | 46 | 25.954 | −9.279 | −18.005 | 1.00 | 10.60 | L |
| ATOM | 350 | CD2 | LEU | L | 46 | 27.187 | −7.973 | −19.750 | 1.00 | 9.16 | L |
| ATOM | 351 | C | LEU | L | 46 | 28.596 | −8.558 | −15.579 | 1.00 | 13.98 | L |
| ATOM | 352 | O | LEU | L | 46 | 29.750 | −8.138 | −15.644 | 1.00 | 12.98 | L |
| ATOM | 353 | N | LEU | L | 47 | 28.295 | −9.820 | −15.284 | 1.00 | 13.60 | L |
| ATOM | 354 | CA | LEU | L | 47 | 29.325 | −10.819 | −14.993 | 1.00 | 14.28 | L |
| ATOM | 355 | CB | LEU | L | 47 | 29.037 | −11.498 | −13.655 | 1.00 | 15.98 | L |
| ATOM | 356 | CG | LEU | L | 47 | 28.957 | −10.647 | −12.383 | 1.00 | 15.79 | L |
| ATOM | 357 | CD1 | LEU | L | 47 | 28.436 | −11.504 | −11.233 | 1.00 | 18.01 | L |
| ATOM | 358 | CD2 | LEU | L | 47 | 30.322 | −10.078 | −12.047 | 1.00 | 15.65 | L |
| ATOM | 359 | C | LEU | L | 47 | 29.369 | −11.894 | −16.069 | 1.00 | 15.50 | L |
| ATOM | 360 | O | LEU | L | 47 | 30.442 | −12.308 | −16.522 | 1.00 | 14.65 | L |
| ATOM | 361 | N | ILE | L | 48 | 28.185 | −12.345 | −16.459 | 1.00 | 13.37 | L |
| ATOM | 362 | CA | ILE | L | 48 | 28.036 | −13.399 | −17.448 | 1.00 | 14.55 | L |
| ATOM | 363 | CB | ILE | L | 48 | 27.750 | −14.760 | −16.749 | 1.00 | 13.22 | L |
| ATOM | 364 | CG2 | ILE | L | 48 | 27.493 | −15.838 | −17.791 | 1.00 | 10.73 | L |
| ATOM | 365 | CG1 | ILE | L | 48 | 28.893 | −15.136 | −15.797 | 1.00 | 12.15 | L |
| ATOM | 366 | CD1 | ILE | L | 48 | 30.168 | −15.598 | −16.480 | 1.00 | 10.81 | L |
| ATOM | 367 | C | ILE | L | 48 | 26.852 | −13.108 | −18.376 | 1.00 | 15.48 | L |
| ATOM | 368 | O | ILE | L | 48 | 25.826 | −12.587 | −17.935 | 1.00 | 14.33 | L |
| ATOM | 369 | N | TYR | L | 49 | 27.000 | −13.443 | −19.655 | 1.00 | 16.97 | L |
| ATOM | 370 | CA | TYR | L | 49 | 25.910 | −13.281 | −20.617 | 1.00 | 17.75 | L |
| ATOM | 371 | CB | TYR | L | 49 | 26.075 | −12.017 | −21.465 | 1.00 | 17.06 | L |
| ATOM | 372 | CG | TYR | L | 49 | 27.287 | −12.002 | −22.359 | 1.00 | 17.09 | L |
| ATOM | 373 | CD1 | TYR | L | 49 | 28.534 | −11.639 | −21.864 | 1.00 | 17.08 | L |
| ATOM | 374 | CE1 | TYR | L | 49 | 29.651 | −11.619 | −22.685 | 1.00 | 17.52 | L |
| ATOM | 375 | CD2 | TYR | L | 49 | 27.185 | −12.351 | −23.702 | 1.00 | 18.93 | L |
| ATOM | 376 | CE2 | TYR | L | 49 | 28.301 | −12.338 | −24.534 | 1.00 | 19.73 | L |
| ATOM | 377 | CZ | TYR | L | 49 | 29.529 | −11.969 | −24.015 | 1.00 | 19.39 | L |
| ATOM | 378 | OH | TYR | L | 49 | 30.640 | −11.951 | −24.826 | 1.00 | 22.53 | L |
| ATOM | 379 | C | TYR | L | 49 | 25.918 | −14.520 | −21.507 | 1.00 | 19.14 | L |
| ATOM | 380 | O | TYR | L | 49 | 26.931 | −15.210 | −21.597 | 1.00 | 19.50 | L |
| ATOM | 381 | N | GLY | L | 50 | 24.794 | −14.815 | −22.150 | 1.00 | 18.39 | L |
| ATOM | 382 | CA | GLY | L | 50 | 24.748 | −15.997 | −22.991 | 1.00 | 18.48 | L |
| ATOM | 383 | C | GLY | L | 50 | 24.995 | −17.255 | −22.175 | 1.00 | 18.64 | L |
| ATOM | 384 | O | GLY | L | 50 | 25.544 | −18.229 | −22.684 | 1.00 | 20.86 | L |
| ATOM | 385 | N | ALA | L | 51 | 24.595 | −17.214 | −20.904 | 1.00 | 17.30 | L |
| ATOM | 386 | CA | ALA | L | 51 | 24.728 | −18.320 | −19.960 | 1.00 | 16.86 | L |
| ATOM | 387 | CB | ALA | L | 51 | 24.050 | −19.592 | −20.522 | 1.00 | 16.18 | L |
| ATOM | 388 | C | ALA | L | 51 | 26.145 | −18.656 | −19.507 | 1.00 | 16.20 | L |
| ATOM | 389 | O | ALA | L | 51 | 26.351 | −19.004 | −18.345 | 1.00 | 15.32 | L |
| ATOM | 390 | N | SER | L | 52 | 27.124 | −18.547 | −20.400 | 1.00 | 17.04 | L |
| ATOM | 391 | CA | SER | L | 52 | 28.487 | −18.907 | −20.025 | 1.00 | 19.91 | L |
| ATOM | 392 | CB | SER | L | 52 | 28.808 | −20.297 | −20.565 | 1.00 | 19.38 | L |
| ATOM | 393 | OG | SER | L | 52 | 28.724 | −20.298 | −21.982 | 1.00 | 23.58 | L |
| ATOM | 394 | C | SER | L | 52 | 29.596 | −17.965 | −20.461 | 1.00 | 20.62 | L |
| ATOM | 395 | O | SER | L | 52 | 30.762 | −18.173 | −20.111 | 1.00 | 19.13 | L |
| ATOM | 396 | N | THR | L | 53 | 29.262 | −16.941 | −21.233 | 1.00 | 20.57 | L |
| ATOM | 397 | CA | THR | L | 53 | 30.304 | −16.031 | −21.683 | 1.00 | 22.76 | L |
| ATOM | 398 | CB | THR | L | 53 | 29.932 | −15.374 | −23.019 | 1.00 | 22.41 | L |
| ATOM | 399 | OG1 | THR | L | 53 | 29.556 | −16.388 | −23.957 | 1.00 | 25.19 | L |
| ATOM | 400 | CG2 | THR | L | 53 | 31.125 | −14.628 | −23.579 | 1.00 | 28.21 | L |
| ATOM | 401 | C | THR | L | 53 | 30.609 | −14.952 | −20.651 | 1.00 | 22.57 | L |
| ATOM | 402 | O | THR | L | 53 | 29.707 | −14.311 | −20.108 | 1.00 | 23.70 | L |
| ATOM | 403 | N | ARG | L | 54 | 31.896 | −14.756 | −20.390 | 1.00 | 21.90 | L |
| ATOM | 404 | CA | ARG | L | 54 | 32.342 | −13.774 | −19.419 | 1.00 | 22.39 | L |
| ATOM | 405 | CB | ARG | L | 54 | 33.318 | −14.426 | −18.439 | 1.00 | 23.94 | L |
| ATOM | 406 | CG | ARG | L | 54 | 33.746 | −13.532 | −17.295 | 1.00 | 22.06 | L |
| ATOM | 407 | CD | ARG | L | 54 | 34.651 | −14.279 | −16.332 | 1.00 | 25.19 | L |
| ATOM | 408 | NE | ARG | L | 54 | 35.833 | −14.791 | −17.014 | 1.00 | 25.41 | L |
| ATOM | 409 | CZ | ARG | L | 54 | 36.133 | −16.080 | −17.135 | 1.00 | 26.46 | L |
| ATOM | 410 | NH1 | ARG | L | 54 | 35.340 | −17.009 | −16.613 | 1.00 | 25.19 | L |
| ATOM | 411 | NH2 | ARG | L | 54 | 37.227 | −16.439 | −17.794 | 1.00 | 24.96 | L |
| ATOM | 412 | C | ARG | L | 54 | 33.022 | −12.615 | −20.122 | 1.00 | 22.64 | L |
| ATOM | 413 | O | ARG | L | 54 | 33.975 | −12.813 | −20.861 | 1.00 | 23.79 | L |
| ATOM | 414 | N | PRO | L | 55 | 32.538 | −11.383 | −19.905 | 1.00 | 23.26 | L |
| ATOM | 415 | CD | PRO | L | 55 | 31.375 | −10.949 | −19.114 | 1.00 | 21.52 | L |
| ATOM | 416 | CA | PRO | L | 55 | 33.170 | −10.241 | −20.565 | 1.00 | 23.60 | L |
| ATOM | 417 | CB | PRO | L | 55 | 32.205 | −9.092 | −20.276 | 1.00 | 21.17 | L |
| ATOM | 418 | CG | PRO | L | 55 | 31.633 | −9.463 | −18.966 | 1.00 | 22.38 | L |
| ATOM | 419 | C | PRO | L | 55 | 34.564 | −9.950 | −20.040 | 1.00 | 24.67 | L |
| ATOM | 420 | O | PRO | L | 55 | 34.882 | −10.243 | −18.890 | 1.00 | 25.87 | L |
| ATOM | 421 | N | SER | L | 56 | 35.395 | −9.377 | −20.899 | 1.00 | 27.72 | L |
| ATOM | 422 | CA | SER | L | 56 | 36.747 | −9.009 | −20.521 | 1.00 | 29.71 | L |
| ATOM | 423 | CB | SER | L | 56 | 37.446 | −8.315 | −21.692 | 1.00 | 32.14 | L |
| ATOM | 424 | OG | SER | L | 56 | 38.498 | −7.483 | −21.236 | 1.00 | 37.03 | L |
| ATOM | 425 | C | SER | L | 56 | 36.650 | −8.052 | −19.335 | 1.00 | 28.66 | L |
| ATOM | 426 | O | SER | L | 56 | 35.864 | −7.101 | −19.359 | 1.00 | 30.71 | L |
| ATOM | 427 | N | GLY | L | 57 | 37.437 | −8.311 | −18.298 | 1.00 | 25.99 | L |
| ATOM | 428 | CA | GLY | L | 57 | 37.407 | −7.452 | −17.130 | 1.00 | 23.46 | L |
| ATOM | 429 | C | GLY | L | 57 | 36.824 | −8.126 | −15.905 | 1.00 | 21.04 | L |
| ATOM | 430 | O | GLY | L | 57 | 37.169 | −7.774 | −14.777 | 1.00 | 23.99 | L |
| ATOM | 431 | N | VAL | L | 58 | 35.936 | −9.092 | −16.119 | 1.00 | 20.09 | L |
| ATOM | 432 | CA | VAL | L | 58 | 35.318 | −9.817 | −15.015 | 1.00 | 18.09 | L |
| ATOM | 433 | CB | VAL | L | 58 | 33.974 | −10.454 | −15.465 | 1.00 | 17.42 | L |
| ATOM | 434 | CG1 | VAL | L | 58 | 33.371 | −11.274 | −14.347 | 1.00 | 13.35 | L |
| ATOM | 435 | CG2 | VAL | L | 58 | 32.999 | −9.356 | −15.875 | 1.00 | 22.20 | L |
| ATOM | 436 | C | VAL | L | 58 | 36.291 | −10.903 | −14.552 | 1.00 | 18.60 | L |
| ATOM | 437 | O | VAL | L | 58 | 36.834 | −11.639 | −15.372 | 1.00 | 18.10 | L |
| ATOM | 438 | N | SER | L | 59 | 36.519 | −11.003 | −13.245 | 1.00 | 18.47 | L |
| ATOM | 439 | CA | SER | L | 59 | 37.450 | −12.006 | −12.736 | 1.00 | 19.98 | L |
| ATOM | 440 | CB | SER | L | 59 | 37.537 | −11.928 | −11.215 | 1.00 | 19.80 | L |
| ATOM | 441 | OG | SER | L | 59 | 36.299 | −12.252 | −10.617 | 1.00 | 33.96 | L |
| ATOM | 442 | C | SER | L | 59 | 37.053 | −13.419 | −13.167 | 1.00 | 17.53 | L |
| ATOM | 443 | O | SER | L | 59 | 35.877 | −13.791 | −13.123 | 1.00 | 16.98 | L |
| ATOM | 444 | N | ASP | L | 60 | 38.041 | −14.205 | −13.582 | 1.00 | 14.16 | L |
| ATOM | 445 | CA | ASP | L | 60 | 37.771 | −15.558 | −14.036 | 1.00 | 15.18 | L |
| ATOM | 446 | CB | ASP | L | 60 | 38.965 | −16.133 | −14.818 | 1.00 | 14.36 | L |
| ATOM | 447 | CG | ASP | L | 60 | 40.239 | −16.227 | −13.991 | 1.00 | 14.95 | L |
| ATOM | 448 | OD1 | ASP | L | 60 | 40.182 | −16.191 | −12.739 | 1.00 | 14.03 | L |
| ATOM | 449 | OD2 | ASP | L | 60 | 41.313 | −16.362 | −14.613 | 1.00 | 18.87 | L |
| ATOM | 450 | C | ASP | L | 60 | 37.350 | −16.532 | −12.951 | 1.00 | 15.28 | L |
| ATOM | 451 | O | ASP | L | 60 | 37.224 | −17.720 | −13.213 | 1.00 | 19.26 | L |
| ATOM | 452 | N | ARG | L | 61 | 37.135 | −16.051 | −11.731 | 1.00 | 16.41 | L |
| ATOM | 453 | CA | ARG | L | 61 | 36.688 | −16.957 | −10.684 | 1.00 | 17.77 | L |
| ATOM | 454 | CB | ARG | L | 61 | 37.116 | −16.465 | −9.291 | 1.00 | 18.44 | L |
| ATOM | 455 | CG | ARG | L | 61 | 36.714 | −15.059 | −8.913 | 1.00 | 17.88 | L |
| ATOM | 456 | CD | ARG | L | 61 | 37.099 | −14.786 | −7.461 | 1.00 | 17.80 | L |
| ATOM | 457 | NE | ARG | L | 61 | 36.684 | −13.457 | −7.037 | 1.00 | 16.49 | L |
| ATOM | 458 | CZ | ARG | L | 61 | 37.305 | −12.334 | −7.384 | 1.00 | 17.70 | L |
| ATOM | 459 | NH1 | ARG | L | 61 | 38.377 | −12.387 | −8.154 | 1.00 | 9.05 | L |
| ATOM | 460 | NH2 | ARG | L | 61 | 36.836 | −11.158 | −6.982 | 1.00 | 15.83 | L |
| ATOM | 461 | C | ARG | L | 61 | 35.165 | −17.113 | −10.779 | 1.00 | 17.33 | L |
| ATOM | 462 | O | ARG | L | 61 | 34.570 | −17.956 | −10.107 | 1.00 | 15.58 | L |
| ATOM | 463 | N | PHE | L | 62 | 34.555 | −16.291 | −11.632 | 1.00 | 15.89 | L |
| ATOM | 464 | CA | PHE | L | 62 | 33.116 | −16.327 | −11.891 | 1.00 | 15.95 | L |
| ATOM | 465 | CB | PHE | L | 62 | 32.543 | −14.912 | −12.110 | 1.00 | 14.45 | L |
| ATOM | 466 | CG | PHE | L | 62 | 32.470 | −14.065 | −10.865 | 1.00 | 14.11 | L |
| ATOM | 467 | CD1 | PHE | L | 62 | 31.429 | −14.223 | −9.956 | 1.00 | 14.61 | L |
| ATOM | 468 | CD2 | PHE | L | 62 | 33.439 | −13.101 | −10.611 | 1.00 | 13.77 | L |
| ATOM | 469 | CE1 | PHE | L | 62 | 31.351 | −13.432 | −8.810 | 1.00 | 15.43 | L |
| ATOM | 470 | CE2 | PHE | L | 62 | 33.373 | −12.302 | −9.466 | 1.00 | 17.30 | L |
| ATOM | 471 | CZ | PHE | L | 62 | 32.327 | −12.467 | −8.563 | 1.00 | 14.02 | L |
| ATOM | 472 | C | PHE | L | 62 | 32.910 | −17.102 | −13.200 | 1.00 | 16.30 | L |
| ATOM | 473 | O | PHE | L | 62 | 33.567 | −16.820 | −14.197 | 1.00 | 15.70 | L |
| ATOM | 474 | N | SER | L | 63 | 32.007 | −18.073 | −13.202 | 1.00 | 14.21 | L |
| ATOM | 475 | CA | SER | L | 63 | 31.720 | −18.807 | −14.430 | 1.00 | 15.54 | L |
| ATOM | 476 | CB | SER | L | 63 | 32.591 | −20.068 | −14.542 | 1.00 | 15.74 | L |
| ATOM | 477 | OG | SER | L | 63 | 32.296 | −20.983 | −13.506 | 1.00 | 17.85 | L |
| ATOM | 478 | C | SER | L | 63 | 30.245 | −19.188 | −14.469 | 1.00 | 15.34 | L |
| ATOM | 479 | O | SER | L | 63 | 29.630 | −19.440 | −13.434 | 1.00 | 13.22 | L |
| ATOM | 480 | N | GLY | L | 64 | 29.677 | −19.219 | −15.670 | 1.00 | 16.11 | L |
| ATOM | 481 | CA | GLY | L | 64 | 28.282 | −19.580 | −15.801 | 1.00 | 17.11 | L |
| ATOM | 482 | C | GLY | L | 64 | 28.119 | −20.850 | −16.613 | 1.00 | 18.62 | L |
| ATOM | 483 | O | GLY | L | 64 | 28.881 | −21.091 | −17.541 | 1.00 | 16.93 | L |
| ATOM | 484 | N | SER | L | 65 | 27.128 | −21.663 | −16.259 | 1.00 | 17.72 | L |
| ATOM | 485 | CA | SER | L | 65 | 26.864 | −22.906 | −16.972 | 1.00 | 18.24 | L |
| ATOM | 486 | CB | SER | L | 65 | 27.586 | −24.075 | −16.289 | 1.00 | 18.76 | L |
| ATOM | 487 | OG | SER | L | 65 | 27.216 | −24.170 | −14.924 | 1.00 | 19.96 | L |
| ATOM | 488 | C | SER | L | 65 | 25.364 | −23.172 | −17.017 | 1.00 | 18.39 | L |
| ATOM | 489 | O | SER | L | 65 | 24.576 | −22.437 | −16.418 | 1.00 | 20.03 | L |
| ATOM | 490 | N | GLY | L | 66 | 24.977 | −24.224 | −17.731 | 1.00 | 18.01 | L |
| ATOM | 491 | CA | GLY | L | 66 | 23.573 | −24.577 | −17.846 | 1.00 | 17.53 | L |
| ATOM | 492 | C | GLY | L | 66 | 22.939 | −24.153 | −19.161 | 1.00 | 19.77 | L |
| ATOM | 493 | O | GLY | L | 66 | 23.548 | −23.435 | −19.961 | 1.00 | 19.20 | L |
| ATOM | 494 | N | SER | L | 67 | 21.710 | −24.610 | −19.386 | 1.00 | 19.48 | L |
| ATOM | 495 | CA | SER | L | 67 | 20.961 | −24.283 | −20.596 | 1.00 | 21.19 | L |
| ATOM | 496 | CB | SER | L | 67 | 21.557 | −25.002 | −21.816 | 1.00 | 21.66 | L |
| ATOM | 497 | OG | SER | L | 67 | 21.716 | −26.387 | −21.580 | 1.00 | 23.72 | L |
| ATOM | 498 | C | SER | L | 67 | 19.494 | −24.663 | −20.412 | 1.00 | 21.36 | L |
| ATOM | 499 | O | SER | L | 67 | 19.141 | −25.341 | −19.452 | 1.00 | 23.13 | L |
| ATOM | 500 | N | GLY | L | 68 | 18.636 | −24.212 | −21.321 | 1.00 | 21.69 | L |
| ATOM | 501 | CA | GLY | L | 68 | 17.223 | −24.523 | −21.202 | 1.00 | 20.28 | L |
| ATOM | 502 | C | GLY | L | 68 | 16.571 | −23.820 | −20.023 | 1.00 | 17.69 | L |
| ATOM | 503 | O | GLY | L | 68 | 16.315 | −22.621 | −20.081 | 1.00 | 17.33 | L |
| ATOM | 504 | N | THR | L | 69 | 16.318 | −24.565 | −18.947 | 1.00 | 17.46 | L |
| ATOM | 505 | CA | THR | L | 69 | 15.685 | −24.022 | −17.742 | 1.00 | 20.27 | L |
| ATOM | 506 | CB | THR | L | 69 | 14.396 | −24.781 | −17.410 | 1.00 | 22.33 | L |
| ATOM | 507 | OG1 | THR | L | 69 | 14.720 | −26.160 | −17.176 | 1.00 | 21.98 | L |
| ATOM | 508 | CG2 | THR | L | 69 | 13.388 | −24.671 | −18.543 | 1.00 | 24.66 | L |
| ATOM | 509 | C | THR | L | 69 | 16.524 | −24.088 | −16.463 | 1.00 | 20.49 | L |
| ATOM | 510 | O | THR | L | 69 | 16.080 | −23.620 | −15.415 | 1.00 | 21.39 | L |
| ATOM | 511 | N | ASP | L | 70 | 17.717 | −24.663 | −16.534 | 1.00 | 20.65 | L |
| ATOM | 512 | CA | ASP | L | 70 | 18.548 | −24.833 | −15.337 | 1.00 | 22.38 | L |
| ATOM | 513 | CB | ASP | L | 70 | 18.698 | −26.341 | −15.070 | 1.00 | 26.67 | L |
| ATOM | 514 | CG | ASP | L | 70 | 19.252 | −26.657 | −13.691 | 1.00 | 32.77 | L |
| ATOM | 515 | OD1 | ASP | L | 70 | 19.459 | −25.729 | −12.885 | 1.00 | 35.48 | L |
| ATOM | 516 | OD2 | ASP | L | 70 | 19.474 | −27.855 | −13.409 | 1.00 | 35.76 | L |
| ATOM | 517 | C | ASP | L | 70 | 19.921 | −24.173 | −15.510 | 1.00 | 21.25 | L |
| ATOM | 518 | O | ASP | L | 70 | 20.776 | −24.677 | −16.236 | 1.00 | 20.75 | L |
| ATOM | 519 | N | PHE | L | 71 | 20.127 | −23.047 | −14.837 | 1.00 | 19.99 | L |
| ATOM | 520 | CA | PHE | L | 71 | 21.387 | −22.322 | −14.951 | 1.00 | 16.53 | L |
| ATOM | 521 | CB | PHE | L | 71 | 21.126 | −20.947 | −15.561 | 1.00 | 17.34 | L |
| ATOM | 522 | CG | PHE | L | 71 | 20.557 | −21.009 | −16.951 | 1.00 | 16.99 | L |
| ATOM | 523 | CD1 | PHE | L | 71 | 21.393 | −21.144 | −18.052 | 1.00 | 15.63 | L |
| ATOM | 524 | CD2 | PHE | L | 71 | 19.179 | −20.967 | −17.155 | 1.00 | 19.56 | L |
| ATOM | 525 | CE1 | PHE | L | 71 | 20.863 | −21.235 | −19.339 | 1.00 | 18.16 | L |
| ATOM | 526 | CE2 | PHE | L | 71 | 18.641 | −21.056 | −18.435 | 1.00 | 18.46 | L |
| ATOM | 527 | CZ | PHE | L | 71 | 19.487 | −21.191 | −19.529 | 1.00 | 19.12 | L |
| ATOM | 528 | C | PHE | L | 71 | 22.098 | −22.174 | −13.622 | 1.00 | 16.87 | L |
| ATOM | 529 | O | PHE | L | 71 | 21.464 | −22.129 | −12.568 | 1.00 | 15.76 | L |
| ATOM | 530 | N | THR | L | 72 | 23.424 | −22.099 | −13.676 | 1.00 | 15.69 | L |
| ATOM | 531 | CA | THR | L | 72 | 24.217 | −21.968 | −12.463 | 1.00 | 15.15 | L |
| ATOM | 532 | CB | THR | L | 72 | 24.861 | −23.336 | −12.046 | 1.00 | 14.09 | L |
| ATOM | 533 | OG1 | THR | L | 72 | 23.839 | −24.300 | −11.779 | 1.00 | 16.77 | L |
| ATOM | 534 | CG2 | THR | L | 72 | 25.714 | −23.165 | −10.796 | 1.00 | 12.99 | L |
| ATOM | 535 | C | THR | L | 72 | 25.342 | −20.945 | −12.590 | 1.00 | 13.26 | L |
| ATOM | 536 | O | THR | L | 72 | 26.033 | −20.877 | −13.605 | 1.00 | 13.52 | L |
| ATOM | 537 | N | LEU | L | 73 | 25.505 | −20.133 | −11.554 | 1.00 | 13.61 | L |
| ATOM | 538 | CA | LEU | L | 73 | 26.591 | −19.164 | −11.522 | 1.00 | 12.86 | L |
| ATOM | 539 | CB | LEU | L | 73 | 26.113 | −17.777 | −11.063 | 1.00 | 11.24 | L |
| ATOM | 540 | CG | LEU | L | 73 | 27.255 | −16.804 | −10.717 | 1.00 | 14.30 | L |
| ATOM | 541 | CD1 | LEU | L | 73 | 28.011 | −16.419 | −11.986 | 1.00 | 15.95 | L |
| ATOM | 542 | CD2 | LEU | L | 73 | 26.698 | −15.542 | −10.042 | 1.00 | 14.51 | L |
| ATOM | 543 | C | LEU | L | 73 | 27.528 | −19.745 | −10.477 | 1.00 | 12.39 | L |
| ATOM | 544 | O | LEU | L | 73 | 27.110 | −20.026 | −9.353 | 1.00 | 14.16 | L |
| ATOM | 545 | N | THR | L | 74 | 28.783 | −19.949 | −10.846 | 1.00 | 9.99 | L |
| ATOM | 546 | CA | THR | L | 74 | 29.745 | −20.500 | −9.907 | 1.00 | 12.01 | L |
| ATOM | 547 | CB | THR | L | 74 | 30.397 | −21.794 | −10.465 | 1.00 | 13.62 | L |
| ATOM | 548 | OG1 | THR | L | 74 | 29.377 | −22.763 | −10.718 | 1.00 | 15.08 | L |
| ATOM | 549 | CG2 | THR | L | 74 | 31.399 | −22.374 | −9.465 | 1.00 | 10.75 | L |
| ATOM | 550 | C | THR | L | 74 | 30.841 | −19.494 | −9.602 | 1.00 | 12.93 | L |
| ATOM | 551 | O | THR | L | 74 | 31.338 | −18.816 | −10.494 | 1.00 | 13.46 | L |
| ATOM | 552 | N | ILE | L | 75 | 31.191 | −19.383 | −8.327 | 1.00 | 14.21 | L |
| ATOM | 553 | CA | ILE | L | 75 | 32.263 | −18.497 | −7.914 | 1.00 | 13.74 | L |
| ATOM | 554 | CB | ILE | L | 75 | 31.778 | −17.447 | −6.896 | 1.00 | 13.62 | L |
| ATOM | 555 | CG2 | ILE | L | 75 | 32.876 | −16.414 | −6.659 | 1.00 | 10.55 | L |
| ATOM | 556 | CG1 | ILE | L | 75 | 30.522 | −16.740 | −7.431 | 1.00 | 14.29 | L |
| ATOM | 557 | CD1 | ILE | L | 75 | 29.927 | −15.711 | −6.478 | 1.00 | 12.24 | L |
| ATOM | 558 | C | ILE | L | 75 | 33.276 | −19.451 | −7.277 | 1.00 | 14.16 | L |
| ATOM | 559 | O | ILE | L | 75 | 33.030 | −20.003 | −6.202 | 1.00 | 12.36 | L |
| ATOM | 560 | N | SER | L | 76 | 34.396 | −19.665 | −7.967 | 1.00 | 16.26 | L |
| ATOM | 561 | CA | SER | L | 76 | 35.426 | −20.596 | −7.501 | 1.00 | 18.41 | L |
| ATOM | 562 | CB | SER | L | 76 | 36.642 | −20.550 | −8.432 | 1.00 | 16.25 | L |
| ATOM | 563 | OG | SER | L | 76 | 37.269 | −19.290 | −8.402 | 1.00 | 21.54 | L |
| ATOM | 564 | C | SER | L | 76 | 35.845 | −20.372 | −6.052 | 1.00 | 20.17 | L |
| ATOM | 565 | O | SER | L | 76 | 35.915 | −21.319 | −5.277 | 1.00 | 20.41 | L |
| ATOM | 566 | N | ARG | L | 77 | 36.125 | −19.122 | −5.690 | 1.00 | 22.06 | L |
| ATOM | 567 | CA | ARG | L | 77 | 36.499 | −18.784 | −4.320 | 1.00 | 23.75 | L |
| ATOM | 568 | CB | ARG | L | 77 | 38.015 | −18.899 | −4.113 | 1.00 | 27.48 | L |
| ATOM | 569 | CG | ARG | L | 77 | 38.874 | −18.036 | −5.021 | 1.00 | 35.32 | L |
| ATOM | 570 | CD | ARG | L | 77 | 39.862 | −17.226 | −4.198 | 1.00 | 38.79 | L |
| ATOM | 571 | NE | ARG | L | 77 | 40.429 | −18.016 | −3.108 | 1.00 | 43.28 | L |
| ATOM | 572 | CZ | ARG | L | 77 | 40.997 | −17.500 | −2.022 | 1.00 | 45.60 | L |
| ATOM | 573 | NH1 | ARG | L | 77 | 41.482 | −18.304 | −1.085 | 1.00 | 45.59 | L |
| ATOM | 574 | NH2 | ARG | L | 77 | 41.076 | −16.183 | −1.864 | 1.00 | 47.79 | L |
| ATOM | 575 | C | ARG | L | 77 | 36.020 | −17.373 | −3.984 | 1.00 | 22.96 | L |
| ATOM | 576 | O | ARG | L | 77 | 36.213 | −16.435 | −4.755 | 1.00 | 23.05 | L |
| ATOM | 577 | N | LEU | L | 78 | 35.394 | −17.225 | −2.823 | 1.00 | 22.27 | L |
| ATOM | 578 | CA | LEU | L | 78 | 34.858 | −15.928 | −2.432 | 1.00 | 23.22 | L |
| ATOM | 579 | CB | LEU | L | 78 | 33.707 | −16.126 | −1.438 | 1.00 | 19.33 | L |
| ATOM | 580 | CG | LEU | L | 78 | 32.438 | −16.795 | −1.990 | 1.00 | 21.78 | L |
| ATOM | 581 | CD1 | LEU | L | 78 | 31.563 | −17.299 | −0.842 | 1.00 | 19.48 | L |
| ATOM | 582 | CD2 | LEU | L | 78 | 31.675 | −15.805 | −2.866 | 1.00 | 18.23 | L |
| ATOM | 583 | C | LEU | L | 78 | 35.859 | −14.925 | −1.867 | 1.00 | 23.05 | L |
| ATOM | 584 | O | LEU | L | 78 | 36.365 | −15.091 | −0.757 | 1.00 | 23.59 | L |
| ATOM | 585 | N | GLN | L | 79 | 36.150 | −13.886 | −2.646 | 1.00 | 23.01 | L |
| ATOM | 586 | CA | GLN | L | 79 | 37.043 | −12.832 | −2.181 | 1.00 | 22.70 | L |
| ATOM | 587 | CB | GLN | L | 79 | 37.643 | −12.045 | −3.351 | 1.00 | 23.82 | L |
| ATOM | 588 | CG | GLN | L | 79 | 38.510 | −12.844 | −4.307 | 1.00 | 27.12 | L |
| ATOM | 589 | CD | GLN | L | 79 | 39.740 | −13.444 | −3.646 | 1.00 | 28.43 | L |
| ATOM | 590 | OE1 | GLN | L | 79 | 40.171 | −13.003 | −2.580 | 1.00 | 30.84 | L |
| ATOM | 591 | NE2 | GLN | L | 79 | 40.322 | −14.447 | −4.292 | 1.00 | 30.37 | L |
| ATOM | 592 | C | GLN | L | 79 | 36.122 | −11.914 | −1.369 | 1.00 | 21.21 | L |
| ATOM | 593 | O | GLN | L | 79 | 34.897 | −11.993 | −1.483 | 1.00 | 20.12 | L |
| ATOM | 594 | N | PRO | L | 80 | 36.697 | −11.034 | −0.540 | 1.00 | 20.77 | L |
| ATOM | 595 | CD | PRO | L | 80 | 38.135 | −10.873 | −0.250 | 1.00 | 20.97 | L |
| ATOM | 596 | CA | PRO | L | 80 | 35.892 | −10.123 | 0.277 | 1.00 | 19.79 | L |
| ATOM | 597 | CB | PRO | L | 80 | 36.946 | −9.231 | 0.934 | 1.00 | 20.60 | L |
| ATOM | 598 | CG | PRO | L | 80 | 38.114 | −10.163 | 1.086 | 1.00 | 22.31 | L |
| ATOM | 599 | C | PRO | L | 80 | 34.861 | −9.309 | −0.500 | 1.00 | 18.61 | L |
| ATOM | 600 | O | PRO | L | 80 | 33.757 | −9.079 | −0.015 | 1.00 | 18.59 | L |
| ATOM | 601 | N | GLU | L | 81 | 35.217 | −8.882 | −1.705 | 1.00 | 18.03 | L |
| ATOM | 602 | CA | GLU | L | 81 | 34.313 | −8.065 | −2.504 | 1.00 | 18.76 | L |
| ATOM | 603 | CB | GLU | L | 81 | 35.110 | −7.192 | −3.487 | 1.00 | 20.87 | L |
| ATOM | 604 | CG | GLU | L | 81 | 35.762 | −7.931 | −4.655 | 1.00 | 25.64 | L |
| ATOM | 605 | CD | GLU | L | 81 | 37.182 | −8.409 | −4.374 | 1.00 | 29.57 | L |
| ATOM | 606 | OE1 | GLU | L | 81 | 37.891 | −8.690 | −5.359 | 1.00 | 32.05 | L |
| ATOM | 607 | OE2 | GLU | L | 81 | 37.597 | −8.514 | −3.195 | 1.00 | 27.43 | L |
| ATOM | 608 | C | GLU | L | 81 | 33.244 | −8.842 | −3.265 | 1.00 | 18.09 | L |
| ATOM | 609 | O | GLU | L | 81 | 32.414 | −8.245 | −3.949 | 1.00 | 16.61 | L |
| ATOM | 610 | N | ASP | L | 82 | 33.254 | −10.167 | −3.139 | 1.00 | 16.32 | L |
| ATOM | 611 | CA | ASP | L | 82 | 32.276 | −10.982 | −3.844 | 1.00 | 16.70 | L |
| ATOM | 612 | CB | ASP | L | 82 | 32.853 | −12.358 | −4.185 | 1.00 | 15.49 | L |
| ATOM | 613 | CG | ASP | L | 82 | 34.033 | −12.272 | −5.124 | 1.00 | 17.53 | L |
| ATOM | 614 | OD1 | ASP | L | 82 | 34.103 | −11.295 | −5.899 | 1.00 | 16.31 | L |
| ATOM | 615 | OD2 | ASP | L | 82 | 34.885 | −13.187 | −5.097 | 1.00 | 18.40 | L |
| ATOM | 616 | C | ASP | L | 82 | 30.990 | −11.152 | −3.058 | 1.00 | 14.79 | L |
| ATOM | 617 | O | ASP | L | 82 | 30.009 | −11.680 | −3.573 | 1.00 | 15.96 | L |
| ATOM | 618 | N | PHE | L | 83 | 31.000 | −10.736 | −1.801 | 1.00 | 13.81 | L |
| ATOM | 619 | CA | PHE | L | 83 | 29.801 | −10.827 | −1.002 | 1.00 | 13.59 | L |
| ATOM | 620 | CB | PHE | L | 83 | 30.154 | −10.795 | 0.488 | 1.00 | 12.87 | L |
| ATOM | 621 | CG | PHE | L | 83 | 30.904 | −12.016 | 0.938 | 1.00 | 12.43 | L |
| ATOM | 622 | CD1 | PHE | L | 83 | 32.296 | −12.051 | 0.912 | 1.00 | 14.24 | L |
| ATOM | 623 | CD2 | PHE | L | 83 | 30.211 | −13.164 | 1.328 | 1.00 | 13.89 | L |
| ATOM | 624 | CE1 | PHE | L | 83 | 32.993 | −13.219 | 1.268 | 1.00 | 11.30 | L |
| ATOM | 625 | CE2 | PHE | L | 83 | 30.896 | −14.336 | 1.685 | 1.00 | 11.70 | L |
| ATOM | 626 | CZ | PHE | L | 83 | 32.290 | −14.359 | 1.653 | 1.00 | 12.81 | L |
| ATOM | 627 | C | PHE | L | 83 | 28.908 | −9.663 | −1.414 | 1.00 | 14.78 | L |
| ATOM | 628 | O | PHE | L | 83 | 29.164 | −8.502 | −1.088 | 1.00 | 14.23 | L |
| ATOM | 629 | N | ALA | L | 84 | 27.873 | −10.000 | −2.173 | 1.00 | 14.58 | L |
| ATOM | 630 | CA | ALA | L | 84 | 26.934 | −9.026 | −2.700 | 1.00 | 15.43 | L |
| ATOM | 631 | CB | ALA | L | 84 | 27.565 | −8.311 | −3.879 | 1.00 | 17.69 | L |
| ATOM | 632 | C | ALA | L | 84 | 25.690 | −9.772 | −3.156 | 1.00 | 16.55 | L |
| ATOM | 633 | O | ALA | L | 84 | 25.497 | −10.931 | −2.802 | 1.00 | 15.47 | L |
| ATOM | 634 | N | THR | L | 85 | 24.855 | −9.108 | −3.952 | 1.00 | 15.49 | L |
| ATOM | 635 | CA | THR | L | 85 | 23.645 | −9.737 | −4.454 | 1.00 | 13.68 | L |
| ATOM | 636 | CB | THR | L | 85 | 22.428 | −8.803 | −4.315 | 1.00 | 16.11 | L |
| ATOM | 637 | OG1 | THR | L | 85 | 22.314 | −8.376 | −2.946 | 1.00 | 17.16 | L |
| ATOM | 638 | CG2 | THR | L | 85 | 21.144 | −9.542 | −4.719 | 1.00 | 10.98 | L |
| ATOM | 639 | C | THR | L | 85 | 23.848 | −10.100 | −5.920 | 1.00 | 13.74 | L |
| ATOM | 640 | O | THR | L | 85 | 24.494 | −9.366 | −6.659 | 1.00 | 10.71 | L |
| ATOM | 641 | N | TYR | L | 86 | 23.296 | −11.240 | −6.326 | 1.00 | 11.81 | L |
| ATOM | 642 | CA | TYR | L | 86 | 23.427 | −11.713 | −7.693 | 1.00 | 11.97 | L |
| ATOM | 643 | CB | TYR | L | 86 | 24.262 | −12.999 | −7.701 | 1.00 | 11.03 | L |
| ATOM | 644 | CG | TYR | L | 86 | 25.697 | −12.769 | −7.275 | 1.00 | 11.30 | L |
| ATOM | 645 | CD1 | TYR | L | 86 | 26.676 | −12.417 | −8.209 | 1.00 | 11.13 | L |
| ATOM | 646 | CE1 | TYR | L | 86 | 27.980 | −12.143 | −7.818 | 1.00 | 12.04 | L |
| ATOM | 647 | CD2 | TYR | L | 86 | 26.067 | −12.841 | −5.927 | 1.00 | 10.17 | L |
| ATOM | 648 | CE2 | TYR | L | 86 | 27.370 | −12.561 | −5.520 | 1.00 | 10.54 | L |
| ATOM | 649 | CZ | TYR | L | 86 | 28.322 | −12.213 | −6.475 | 1.00 | 11.86 | L |
| ATOM | 650 | OH | TYR | L | 86 | 29.608 | −11.923 | −6.089 | 1.00 | 12.48 | L |
| ATOM | 651 | C | TYR | L | 86 | 22.063 | −11.943 | −8.352 | 1.00 | 14.23 | L |
| ATOM | 652 | O | TYR | L | 86 | 21.199 | −12.635 | −7.813 | 1.00 | 14.83 | L |
| ATOM | 653 | N | TYR | L | 87 | 21.878 | −11.343 | −9.522 | 1.00 | 14.68 | L |
| ATOM | 654 | CA | TYR | L | 87 | 20.625 | −11.476 | −10.252 | 1.00 | 12.98 | L |
| ATOM | 655 | CB | TYR | L | 87 | 20.025 | −10.093 | −10.576 | 1.00 | 12.27 | L |
| ATOM | 656 | CG | TYR | L | 87 | 19.564 | −9.272 | −9.389 | 1.00 | 13.32 | L |
| ATOM | 657 | CD1 | TYR | L | 87 | 18.329 | −9.504 | −8.790 | 1.00 | 11.45 | L |
| ATOM | 658 | CE1 | TYR | L | 87 | 17.915 | −8.765 | −7.680 | 1.00 | 11.35 | L |
| ATOM | 659 | CD2 | TYR | L | 87 | 20.379 | −8.274 | −8.852 | 1.00 | 14.90 | L |
| ATOM | 660 | CE2 | TYR | L | 87 | 19.976 | −7.529 | −7.743 | 1.00 | 13.16 | L |
| ATOM | 661 | CZ | TYR | L | 87 | 18.745 | −7.784 | −7.162 | 1.00 | 13.28 | L |
| ATOM | 662 | OH | TYR | L | 87 | 18.361 | −7.080 | −6.043 | 1.00 | 14.53 | L |
| ATOM | 663 | C | TYR | L | 87 | 20.851 | −12.190 | −11.573 | 1.00 | 13.16 | L |
| ATOM | 664 | O | TYR | L | 87 | 21.863 | −11.968 | −12.244 | 1.00 | 13.55 | L |
| ATOM | 665 | N | CYS | L | 88 | 19.936 | −13.077 | −11.933 | 1.00 | 11.42 | L |
| ATOM | 666 | CA | CYS | L | 88 | 20.035 | −13.686 | −13.238 | 1.00 | 14.30 | L |
| ATOM | 667 | C | CYS | L | 88 | 18.959 | −12.942 | −14.038 | 1.00 | 14.23 | L |
| ATOM | 668 | O | CYS | L | 88 | 18.068 | −12.311 | −13.465 | 1.00 | 14.06 | L |
| ATOM | 669 | CB | CYS | L | 88 | 19.775 | −15.200 | −13.214 | 1.00 | 14.70 | L |
| ATOM | 670 | SG | CYS | L | 88 | 18.231 | −15.802 | −12.475 | 1.00 | 18.56 | L |
| ATOM | 671 | N | GLN | L | 89 | 19.082 | −12.977 | −15.354 | 1.00 | 14.09 | L |
| ATOM | 672 | CA | GLN | L | 89 | 18.139 | −12.308 | −16.233 | 1.00 | 14.30 | L |
| ATOM | 673 | CB | GLN | L | 89 | 18.599 | −10.881 | −16.558 | 1.00 | 15.45 | L |
| ATOM | 674 | CG | GLN | L | 89 | 17.866 | −10.287 | −17.774 | 1.00 | 14.42 | L |
| ATOM | 675 | CD | GLN | L | 89 | 18.701 | −9.269 | −18.536 | 1.00 | 17.10 | L |
| ATOM | 676 | OE1 | GLN | L | 89 | 19.929 | −9.368 | −18.590 | 1.00 | 16.89 | L |
| ATOM | 677 | NE2 | GLN | L | 89 | 18.037 | −8.298 | −19.145 | 1.00 | 16.00 | L |
| ATOM | 678 | C | GLN | L | 89 | 18.047 | −13.074 | −17.534 | 1.00 | 13.48 | L |
| ATOM | 679 | O | GLN | L | 89 | 19.057 | −13.544 | −18.054 | 1.00 | 14.78 | L |
| ATOM | 680 | N | GLN | L | 90 | 16.836 | −13.210 | −18.057 | 1.00 | 13.31 | L |
| ATOM | 681 | CA | GLN | L | 90 | 16.669 | −13.888 | −19.333 | 1.00 | 13.82 | L |
| ATOM | 682 | CB | GLN | L | 90 | 15.680 | −15.066 | −19.216 | 1.00 | 12.97 | L |
| ATOM | 683 | CG | GLN | L | 90 | 14.186 | −14.736 | −19.041 | 1.00 | 12.68 | L |
| ATOM | 684 | CD | GLN | L | 90 | 13.553 | −14.130 | −20.291 | 1.00 | 15.52 | L |
| ATOM | 685 | OE1 | GLN | L | 90 | 13.956 | −14.427 | −21.415 | 1.00 | 13.14 | L |
| ATOM | 686 | NE2 | GLN | L | 90 | 12.544 | −13.291 | −20.094 | 1.00 | 13.86 | L |
| ATOM | 687 | C | GLN | L | 90 | 16.195 | −12.859 | −20.359 | 1.00 | 14.53 | L |
| ATOM | 688 | O | GLN | L | 90 | 15.346 | −12.012 | −20.064 | 1.00 | 13.04 | L |
| ATOM | 689 | N | TYR | L | 91 | 16.784 | −12.908 | −21.548 | 1.00 | 14.58 | L |
| ATOM | 690 | CA | TYR | L | 91 | 16.411 | −12.000 | −22.625 | 1.00 | 16.97 | L |
| ATOM | 691 | CB | TYR | L | 91 | 17.437 | −10.855 | −22.774 | 1.00 | 16.23 | L |
| ATOM | 692 | CG | TYR | L | 91 | 18.884 | −11.299 | −22.810 | 1.00 | 18.28 | L |
| ATOM | 693 | CD1 | TYR | L | 91 | 19.526 | −11.755 | −21.657 | 1.00 | 17.31 | L |
| ATOM | 694 | CE1 | TYR | L | 91 | 20.843 | −12.222 | −21.700 | 1.00 | 18.24 | L |
| ATOM | 695 | CD2 | TYR | L | 91 | 19.600 | −11.311 | −24.011 | 1.00 | 19.27 | L |
| ATOM | 696 | CE2 | TYR | L | 91 | 20.912 | −11.772 | −24.065 | 1.00 | 17.70 | L |
| ATOM | 697 | CZ | TYR | L | 91 | 21.528 | −12.230 | −22.908 | 1.00 | 19.37 | L |
| ATOM | 698 | OH | TYR | L | 91 | 22.819 | −12.711 | −22.970 | 1.00 | 20.68 | L |
| ATOM | 699 | C | TYR | L | 91 | 16.296 | −12.811 | −23.909 | 1.00 | 16.33 | L |
| ATOM | 700 | O | TYR | L | 91 | 16.657 | −12.356 | −24.989 | 1.00 | 16.32 | L |
| ATOM | 701 | N | GLY | L | 92 | 15.774 | −14.027 | −23.753 | 1.00 | 18.40 | L |
| ATOM | 702 | CA | GLY | L | 92 | 15.578 | −14.937 | −24.867 | 1.00 | 19.50 | L |
| ATOM | 703 | C | GLY | L | 92 | 14.271 | −14.674 | −25.595 | 1.00 | 19.66 | L |
| ATOM | 704 | O | GLY | L | 92 | 13.886 | −15.414 | −26.491 | 1.00 | 20.84 | L |
| ATOM | 705 | N | GLN | L | 93 | 13.568 | −13.630 | −25.176 | 1.00 | 21.12 | L |
| ATOM | 706 | CA | GLN | L | 93 | 12.330 | −13.218 | −25.824 | 1.00 | 21.05 | L |
| ATOM | 707 | CB | GLN | L | 93 | 11.137 | −14.068 | −25.361 | 1.00 | 22.34 | L |
| ATOM | 708 | CG | GLN | L | 93 | 10.683 | −13.883 | −23.934 | 1.00 | 23.68 | L |
| ATOM | 709 | CD | GLN | L | 93 | 9.573 | −14.848 | −23.582 | 1.00 | 27.18 | L |
| ATOM | 710 | OE1 | GLN | L | 93 | 8.802 | −14.623 | −22.655 | 1.00 | 30.69 | L |
| ATOM | 711 | NE2 | GLN | L | 93 | 9.493 | −15.942 | −24.324 | 1.00 | 27.89 | L |
| ATOM | 712 | C | GLN | L | 93 | 12.158 | −11.741 | −25.478 | 1.00 | 21.62 | L |
| ATOM | 713 | O | GLN | L | 93 | 12.803 | −11.248 | −24.546 | 1.00 | 20.19 | L |
| ATOM | 714 | N | SER | L | 94 | 11.317 | −11.037 | −26.232 | 1.00 | 20.96 | L |
| ATOM | 715 | CA | SER | L | 94 | 11.126 | −9.604 | −26.037 | 1.00 | 20.63 | L |
| ATOM | 716 | CB | SER | L | 94 | 10.038 | −9.086 | −26.980 | 1.00 | 22.20 | L |
| ATOM | 717 | OG | SER | L | 94 | 10.428 | −9.287 | −28.333 | 1.00 | 23.94 | L |
| ATOM | 718 | C | SER | L | 94 | 10.847 | −9.162 | −24.610 | 1.00 | 19.48 | L |
| ATOM | 719 | O | SER | L | 94 | 11.353 | −8.130 | −24.176 | 1.00 | 18.14 | L |
| ATOM | 720 | N | LEU | L | 95 | 10.046 | −9.928 | −23.879 | 1.00 | 19.77 | L |
| ATOM | 721 | CA | LEU | L | 95 | 9.765 | −9.582 | −22.490 | 1.00 | 18.39 | L |
| ATOM | 722 | CB | LEU | L | 95 | 8.446 | −10.203 | −22.023 | 1.00 | 20.66 | L |
| ATOM | 723 | CG | LEU | L | 95 | 7.713 | −9.548 | −20.845 | 1.00 | 22.74 | L |
| ATOM | 724 | CD1 | LEU | L | 95 | 6.759 | −10.558 | −20.231 | 1.00 | 19.05 | L |
| ATOM | 725 | CD2 | LEU | L | 95 | 8.686 | −9.052 | −19.796 | 1.00 | 22.43 | L |
| ATOM | 726 | C | LEU | L | 95 | 10.905 | −10.148 | −21.643 | 1.00 | 18.48 | L |
| ATOM | 727 | O | LEU | L | 95 | 10.919 | −11.341 | −21.326 | 1.00 | 16.54 | L |
| ATOM | 728 | N | SER | L | 96 | 11.863 | −9.297 | −21.292 | 1.00 | 16.30 | L |
| ATOM | 729 | CA | SER | L | 96 | 12.986 | −9.726 | −20.466 | 1.00 | 16.74 | L |
| ATOM | 730 | CB | SER | L | 96 | 14.178 | −8.787 | −20.673 | 1.00 | 17.15 | L |
| ATOM | 731 | OG | SER | L | 96 | 15.251 | −9.121 | −19.806 | 1.00 | 13.35 | L |
| ATOM | 732 | C | SER | L | 96 | 12.578 | −9.714 | −18.989 | 1.00 | 16.23 | L |
| ATOM | 733 | O | SER | L | 96 | 11.779 | −8.879 | −18.572 | 1.00 | 16.27 | L |
| ATOM | 734 | N | THR | L | 97 | 13.117 | −10.646 | −18.208 | 1.00 | 15.31 | L |
| ATOM | 735 | CA | THR | L | 97 | 12.824 | −10.710 | −16.776 | 1.00 | 13.73 | L |
| ATOM | 736 | CB | THR | L | 97 | 11.739 | −11.765 | −16.429 | 1.00 | 14.22 | L |
| ATOM | 737 | OG1 | THR | L | 97 | 12.113 | −13.032 | −16.983 | 1.00 | 16.75 | L |
| ATOM | 738 | CG2 | THR | L | 97 | 10.375 | −11.342 | −16.961 | 1.00 | 16.25 | L |
| ATOM | 739 | C | THR | L | 97 | 14.065 | −11.067 | −15.969 | 1.00 | 14.57 | L |
| ATOM | 740 | O | THR | L | 97 | 14.986 | −11.723 | −16.472 | 1.00 | 11.95 | L |
| ATOM | 741 | N | PHE | L | 98 | 14.055 | −10.642 | −14.706 | 1.00 | 14.19 | L |
| ATOM | 742 | CA | PHE | L | 98 | 15.135 | −10.884 | −13.756 | 1.00 | 13.14 | L |
| ATOM | 743 | CB | PHE | L | 98 | 15.538 | −9.585 | −13.036 | 1.00 | 12.49 | L |
| ATOM | 744 | CG | PHE | L | 98 | 16.446 | −8.687 | −13.819 | 1.00 | 13.49 | L |
| ATOM | 745 | CD1 | PHE | L | 98 | 17.823 | −8.857 | −13.775 | 1.00 | 13.27 | L |
| ATOM | 746 | CD2 | PHE | L | 98 | 15.926 | −7.638 | −14.575 | 1.00 | 15.70 | L |
| ATOM | 747 | CE1 | PHE | L | 98 | 18.670 | −7.995 | −14.471 | 1.00 | 16.14 | L |
| ATOM | 748 | CE2 | PHE | L | 98 | 16.770 | −6.771 | −15.272 | 1.00 | 17.19 | L |
| ATOM | 749 | CZ | PHE | L | 98 | 18.141 | −6.950 | −15.221 | 1.00 | 15.24 | L |
| ATOM | 750 | C | PHE | L | 98 | 14.612 | −11.819 | −12.678 | 1.00 | 15.15 | L |
| ATOM | 751 | O | PHE | L | 98 | 13.416 | −11.851 | −12.403 | 1.00 | 13.95 | L |
| ATOM | 752 | N | GLY | L | 99 | 15.517 | −12.573 | −12.063 | 1.00 | 15.54 | L |
| ATOM | 753 | CA | GLY | L | 99 | 15.121 | −13.411 | −10.952 | 1.00 | 14.42 | L |
| ATOM | 754 | C | GLY | L | 99 | 15.101 | −12.449 | −9.766 | 1.00 | 13.63 | L |
| ATOM | 755 | O | GLY | L | 99 | 15.535 | −11.303 | −9.893 | 1.00 | 10.96 | L |
| ATOM | 756 | N | GLN | L | 100 | 14.632 | −12.896 | −8.610 | 1.00 | 13.36 | L |
| ATOM | 757 | CA | GLN | L | 100 | 14.561 | −12.020 | −7.452 | 1.00 | 15.30 | L |
| ATOM | 758 | CB | GLN | L | 100 | 13.543 | −12.574 | −6.453 | 1.00 | 19.93 | L |
| ATOM | 759 | CG | GLN | L | 100 | 12.152 | −12.767 | −7.060 | 1.00 | 25.78 | L |
| ATOM | 760 | CD | GLN | L | 100 | 11.430 | −11.455 | −7.316 | 1.00 | 32.63 | L |
| ATOM | 761 | OE1 | GLN | L | 100 | 12.028 | −10.476 | −7.771 | 1.00 | 32.80 | L |
| ATOM | 762 | NE2 | GLN | L | 100 | 10.131 | −11.435 | −7.036 | 1.00 | 36.81 | L |
| ATOM | 763 | C | GLN | L | 100 | 15.908 | −11.787 | −6.769 | 1.00 | 15.41 | L |
| ATOM | 764 | O | GLN | L | 100 | 15.996 | −11.018 | −5.822 | 1.00 | 12.83 | L |
| ATOM | 765 | N | GLY | L | 101 | 16.951 | −12.464 | −7.239 | 1.00 | 15.67 | L |
| ATOM | 766 | CA | GLY | L | 101 | 18.268 | −12.264 | −6.665 | 1.00 | 14.62 | L |
| ATOM | 767 | C | GLY | L | 101 | 18.658 | −13.103 | −5.458 | 1.00 | 15.32 | L |
| ATOM | 768 | O | GLY | L | 101 | 17.829 | −13.450 | −4.617 | 1.00 | 13.18 | L |
| ATOM | 769 | N | THR | L | 102 | 19.941 | −13.438 | −5.381 | 1.00 | 15.09 | L |
| ATOM | 770 | CA | THR | L | 102 | 20.455 | −14.212 | −4.262 | 1.00 | 14.24 | L |
| ATOM | 771 | CB | THR | L | 102 | 21.064 | −15.557 | −4.723 | 1.00 | 14.43 | L |
| ATOM | 772 | OG1 | THR | L | 102 | 20.014 | −16.446 | −5.121 | 1.00 | 17.70 | L |
| ATOM | 773 | CG2 | THR | L | 102 | 21.873 | −16.197 | −3.593 | 1.00 | 14.71 | L |
| ATOM | 774 | C | THR | L | 102 | 21.538 | −13.397 | −3.573 | 1.00 | 13.18 | L |
| ATOM | 775 | O | THR | L | 102 | 22.480 | −12.935 | −4.215 | 1.00 | 12.72 | L |
| ATOM | 776 | N | LYS | L | 103 | 21.395 | −13.209 | −2.268 | 1.00 | 12.04 | L |
| ATOM | 777 | CA | LYS | L | 103 | 22.392 | −12.466 | −1.513 | 1.00 | 13.04 | L |
| ATOM | 778 | CB | LYS | L | 103 | 21.728 | −11.670 | −0.389 | 1.00 | 13.56 | L |
| ATOM | 779 | CG | LYS | L | 103 | 22.701 | −10.815 | 0.396 | 1.00 | 15.43 | L |
| ATOM | 780 | CD | LYS | L | 103 | 22.047 | −10.166 | 1.595 | 1.00 | 16.18 | L |
| ATOM | 781 | CE | LYS | L | 103 | 23.057 | −9.337 | 2.366 | 1.00 | 18.94 | L |
| ATOM | 782 | NZ | LYS | L | 103 | 22.528 | −8.945 | 3.695 | 1.00 | 23.17 | L |
| ATOM | 783 | C | LYS | L | 103 | 23.420 | −13.417 | −0.900 | 1.00 | 13.45 | L |
| ATOM | 784 | O | LYS | L | 103 | 23.065 | −14.314 | −0.139 | 1.00 | 11.39 | L |
| ATOM | 785 | N | VAL | L | 104 | 24.690 | −13.231 | −1.243 | 1.00 | 12.91 | L |
| ATOM | 786 | CA | VAL | L | 104 | 25.736 | −14.059 | −0.658 | 1.00 | 12.40 | L |
| ATOM | 787 | CB | VAL | L | 104 | 26.812 | −14.443 | −1.688 | 1.00 | 12.24 | L |
| ATOM | 788 | CG1 | VAL | L | 104 | 27.879 | −15.309 | −1.022 | 1.00 | 10.31 | L |
| ATOM | 789 | CG2 | VAL | L | 104 | 26.173 | −15.167 | −2.859 | 1.00 | 10.49 | L |
| ATOM | 790 | C | VAL | L | 104 | 26.385 | −13.232 | 0.445 | 1.00 | 14.22 | L |
| ATOM | 791 | O | VAL | L | 104 | 27.029 | −12.217 | 0.169 | 1.00 | 13.16 | L |
| ATOM | 792 | N | GLU | L | 105 | 26.196 | −13.648 | 1.695 | 1.00 | 13.59 | L |
| ATOM | 793 | CA | GLU | L | 105 | 26.781 | −12.923 | 2.812 | 1.00 | 15.03 | L |
| ATOM | 794 | CB | GLU | L | 105 | 25.697 | −12.469 | 3.785 | 1.00 | 18.65 | L |
| ATOM | 795 | CG | GLU | L | 105 | 24.806 | −13.580 | 4.271 | 1.00 | 24.06 | L |
| ATOM | 796 | CD | GLU | L | 105 | 24.698 | −13.603 | 5.770 | 1.00 | 24.91 | L |
| ATOM | 797 | OE1 | GLU | L | 105 | 24.391 | −12.547 | 6.359 | 1.00 | 27.32 | L |
| ATOM | 798 | OE2 | GLU | L | 105 | 24.916 | −14.677 | 6.361 | 1.00 | 25.84 | L |
| ATOM | 799 | C | GLU | L | 105 | 27.820 | −13.768 | 3.538 | 1.00 | 16.43 | L |
| ATOM | 800 | O | GLU | L | 105 | 27.929 | −14.977 | 3.300 | 1.00 | 14.69 | L |
| ATOM | 801 | N | ILE | L | 106 | 28.582 | −13.122 | 4.422 | 1.00 | 15.30 | L |
| ATOM | 802 | CA | ILE | L | 106 | 29.641 | −13.790 | 5.171 | 1.00 | 14.06 | L |
| ATOM | 803 | CB | ILE | L | 106 | 30.724 | −12.790 | 5.685 | 1.00 | 14.67 | L |
| ATOM | 804 | CG2 | ILE | L | 106 | 31.812 | −13.527 | 6.450 | 1.00 | 10.56 | L |
| ATOM | 805 | CG1 | ILE | L | 106 | 31.353 | −12.048 | 4.511 | 1.00 | 12.89 | L |
| ATOM | 806 | CD1 | ILE | L | 106 | 30.451 | −11.010 | 3.949 | 1.00 | 20.67 | L |
| ATOM | 807 | C | ILE | L | 106 | 29.122 | −14.548 | 6.367 | 1.00 | 14.60 | L |
| ATOM | 808 | O | ILE | L | 106 | 28.441 | −13.989 | 7.227 | 1.00 | 14.28 | L |
| ATOM | 809 | N | ASN | L | 107 | 29.455 | −15.832 | 6.410 | 1.00 | 15.26 | L |
| ATOM | 810 | CA | ASN | L | 107 | 29.053 | −16.684 | 7.511 | 1.00 | 17.09 | L |
| ATOM | 811 | CB | ASN | L | 107 | 29.053 | −18.147 | 7.067 | 1.00 | 20.44 | L |
| ATOM | 812 | CG | ASN | L | 107 | 28.610 | −19.093 | 8.163 | 1.00 | 26.30 | L |
| ATOM | 813 | OD1 | ASN | L | 107 | 28.530 | −18.713 | 9.329 | 1.00 | 31.26 | L |
| ATOM | 814 | ND2 | ASN | L | 107 | 28.328 | −20.342 | 7.793 | 1.00 | 27.45 | L |
| ATOM | 815 | C | ASN | L | 107 | 30.097 | −16.469 | 8.594 | 1.00 | 15.73 | L |
| ATOM | 816 | O | ASN | L | 107 | 31.289 | −16.438 | 8.312 | 1.00 | 18.11 | L |
| ATOM | 817 | N | ARG | L | 108 | 29.652 | −16.304 | 9.831 | 1.00 | 16.88 | L |
| ATOM | 818 | CA | ARG | L | 108 | 30.577 | −16.101 | 10.938 | 1.00 | 17.68 | L |
| ATOM | 819 | CB | ARG | L | 108 | 30.890 | −14.602 | 11.103 | 1.00 | 17.33 | L |
| ATOM | 820 | CG | ARG | L | 108 | 29.682 | −13.742 | 11.445 | 1.00 | 19.57 | L |
| ATOM | 821 | CD | ARG | L | 108 | 29.643 | −13.462 | 12.939 | 1.00 | 23.70 | L |
| ATOM | 822 | NE | ARG | L | 108 | 30.516 | −12.350 | 13.272 | 1.00 | 23.21 | L |
| ATOM | 823 | CZ | ARG | L | 108 | 31.050 | −12.119 | 14.467 | 1.00 | 21.35 | L |
| ATOM | 824 | NH1 | ARG | L | 108 | 30.818 | −12.930 | 15.492 | 1.00 | 23.73 | L |
| ATOM | 825 | NH2 | ARG | L | 108 | 31.813 | −11.053 | 14.630 | 1.00 | 19.49 | L |
| ATOM | 826 | C | ARG | L | 108 | 29.935 | −16.676 | 12.191 | 1.00 | 19.13 | L |
| ATOM | 827 | O | ARG | L | 108 | 28.777 | −17.085 | 12.158 | 1.00 | 20.31 | L |
| ATOM | 828 | N | THR | L | 109 | 30.688 | −16.722 | 13.286 | 1.00 | 20.96 | L |
| ATOM | 829 | CA | THR | L | 109 | 30.180 | −17.268 | 14.540 | 1.00 | 20.73 | L |
| ATOM | 830 | CB | THR | L | 109 | 31.272 | −17.270 | 15.625 | 1.00 | 21.97 | L |
| ATOM | 831 | OG1 | THR | L | 109 | 31.728 | −15.930 | 15.840 | 1.00 | 23.60 | L |
| ATOM | 832 | CG2 | THR | L | 109 | 32.452 | −18.134 | 15.199 | 1.00 | 21.92 | L |
| ATOM | 833 | C | THR | L | 109 | 28.991 | −16.481 | 15.072 | 1.00 | 20.05 | L |
| ATOM | 834 | O | THR | L | 109 | 28.945 | −15.260 | 14.963 | 1.00 | 22.57 | L |
| ATOM | 835 | N | VAL | L | 110 | 28.028 | −17.182 | 15.653 | 1.00 | 17.67 | L |
| ATOM | 836 | CA | VAL | L | 110 | 26.863 | −16.521 | 16.206 | 1.00 | 17.89 | L |
| ATOM | 837 | CB | VAL | L | 110 | 25.930 | −17.544 | 16.893 | 1.00 | 18.94 | L |
| ATOM | 838 | CG1 | VAL | L | 110 | 24.855 | −16.825 | 17.684 | 1.00 | 16.27 | L |
| ATOM | 839 | CG2 | VAL | L | 110 | 25.289 | −18.450 | 15.832 | 1.00 | 17.42 | L |
| ATOM | 840 | C | VAL | L | 110 | 27.278 | −15.439 | 17.216 | 1.00 | 19.74 | L |
| ATOM | 841 | O | VAL | L | 110 | 28.214 | −15.624 | 18.004 | 1.00 | 19.29 | L |
| ATOM | 842 | N | ALA | L | 111 | 26.588 | −14.302 | 17.171 | 1.00 | 16.88 | L |
| ATOM | 843 | CA | ALA | L | 111 | 26.868 | −13.200 | 18.080 | 1.00 | 15.95 | L |
| ATOM | 844 | CB | ALA | L | 111 | 27.760 | −12.158 | 17.402 | 1.00 | 16.40 | L |
| ATOM | 845 | C | ALA | L | 111 | 25.556 | −12.563 | 18.507 | 1.00 | 17.34 | L |
| ATOM | 846 | O | ALA | L | 111 | 24.796 | −12.056 | 17.676 | 1.00 | 15.46 | L |
| ATOM | 847 | N | ALA | L | 112 | 25.288 | −12.598 | 19.806 | 1.00 | 16.64 | L |
| ATOM | 848 | CA | ALA | L | 112 | 24.072 | −12.013 | 20.337 | 1.00 | 18.04 | L |
| ATOM | 849 | CB | ALA | L | 112 | 23.833 | −12.499 | 21.760 | 1.00 | 18.69 | L |
| ATOM | 850 | C | ALA | L | 112 | 24.205 | −10.495 | 20.315 | 1.00 | 18.33 | L |
| ATOM | 851 | O | ALA | L | 112 | 25.293 | −9.952 | 20.448 | 1.00 | 18.79 | L |
| ATOM | 852 | N | PRO | L | 113 | 23.088 | −9.789 | 20.148 | 1.00 | 18.77 | L |
| ATOM | 853 | CD | PRO | L | 113 | 21.713 | −10.268 | 19.919 | 1.00 | 14.09 | L |
| ATOM | 854 | CA | PRO | L | 113 | 23.152 | −8.327 | 20.120 | 1.00 | 18.94 | L |
| ATOM | 855 | CB | PRO | L | 113 | 21.798 | −7.938 | 19.547 | 1.00 | 17.60 | L |
| ATOM | 856 | CG | PRO | L | 113 | 20.897 | −9.006 | 20.109 | 1.00 | 20.83 | L |
| ATOM | 857 | C | PRO | L | 113 | 23.360 | −7.680 | 21.485 | 1.00 | 19.48 | L |
| ATOM | 858 | O | PRO | L | 113 | 22.924 | −8.209 | 22.508 | 1.00 | 19.18 | L |
| ATOM | 859 | N | SER | L | 114 | 24.047 | −6.542 | 21.490 | 1.00 | 18.64 | L |
| ATOM | 860 | CA | SER | L | 114 | 24.225 | −5.765 | 22.709 | 1.00 | 17.61 | L |
| ATOM | 861 | CB | SER | L | 114 | 25.549 | −4.980 | 22.696 | 1.00 | 17.83 | L |
| ATOM | 862 | OG | SER | L | 114 | 26.656 | −5.854 | 22.790 | 1.00 | 25.46 | L |
| ATOM | 863 | C | SER | L | 114 | 23.046 | −4.812 | 22.546 | 1.00 | 14.87 | L |
| ATOM | 864 | O | SER | L | 114 | 22.925 | −4.148 | 21.514 | 1.00 | 13.21 | L |
| ATOM | 865 | N | VAL | L | 115 | 22.183 | −4.755 | 23.552 | 1.00 | 15.89 | L |
| ATOM | 866 | CA | VAL | L | 115 | 20.989 | −3.926 | 23.500 | 1.00 | 14.46 | L |
| ATOM | 867 | CB | VAL | L | 115 | 19.742 | −4.751 | 23.936 | 1.00 | 14.19 | L |
| ATOM | 868 | CG1 | VAL | L | 115 | 18.454 | −4.001 | 23.572 | 1.00 | 9.92 | L |
| ATOM | 869 | CG2 | VAL | L | 115 | 19.780 | −6.135 | 23.279 | 1.00 | 13.35 | L |
| ATOM | 870 | C | VAL | L | 115 | 21.081 | −2.673 | 24.364 | 1.00 | 15.62 | L |
| ATOM | 871 | O | VAL | L | 115 | 21.540 | −2.718 | 25.505 | 1.00 | 15.25 | L |
| ATOM | 872 | N | PHE | L | 116 | 20.628 | −1.556 | 23.805 | 1.00 | 15.06 | L |
| ATOM | 873 | CA | PHE | L | 116 | 20.638 | −0.274 | 24.494 | 1.00 | 14.99 | L |
| ATOM | 874 | CB | PHE | L | 116 | 21.747 | 0.625 | 23.937 | 1.00 | 14.93 | L |
| ATOM | 875 | CG | PHE | L | 116 | 23.128 | 0.042 | 24.051 | 1.00 | 17.15 | L |
| ATOM | 876 | CD1 | PHE | L | 116 | 23.905 | 0.269 | 25.188 | 1.00 | 16.62 | L |
| ATOM | 877 | CD2 | PHE | L | 116 | 23.662 | −0.716 | 23.012 | 1.00 | 14.46 | L |
| ATOM | 878 | CE1 | PHE | L | 116 | 25.199 | −0.247 | 25.287 | 1.00 | 14.65 | L |
| ATOM | 879 | CE2 | PHE | L | 116 | 24.955 | −1.237 | 23.101 | 1.00 | 15.39 | L |
| ATOM | 880 | CZ | PHE | L | 116 | 25.725 | −1.000 | 24.243 | 1.00 | 14.49 | L |
| ATOM | 881 | C | PHE | L | 116 | 19.300 | 0.423 | 24.269 | 1.00 | 15.47 | L |
| ATOM | 882 | O | PHE | L | 116 | 18.734 | 0.343 | 23.181 | 1.00 | 15.22 | L |
| ATOM | 883 | N | ILE | L | 117 | 18.792 | 1.095 | 25.295 | 1.00 | 14.58 | L |
| ATOM | 884 | CA | ILE | L | 117 | 17.546 | 1.830 | 25.152 | 1.00 | 16.07 | L |
| ATOM | 885 | CB | ILE | L | 117 | 16.425 | 1.260 | 26.066 | 1.00 | 16.13 | L |
| ATOM | 886 | CG2 | ILE | L | 117 | 16.721 | 1.569 | 27.543 | 1.00 | 12.80 | L |
| ATOM | 887 | CG1 | ILE | L | 117 | 15.075 | 1.853 | 25.639 | 1.00 | 14.80 | L |
| ATOM | 888 | CD1 | ILE | L | 117 | 13.856 | 1.195 | 26.289 | 1.00 | 14.71 | L |
| ATOM | 889 | C | ILE | L | 117 | 17.810 | 3.294 | 25.495 | 1.00 | 15.53 | L |
| ATOM | 890 | O | ILE | L | 117 | 18.533 | 3.596 | 26.438 | 1.00 | 16.41 | L |
| ATOM | 891 | N | PHE | L | 118 | 17.248 | 4.201 | 24.704 | 1.00 | 16.11 | L |
| ATOM | 892 | CA | PHE | L | 118 | 17.437 | 5.626 | 24.939 | 1.00 | 15.02 | L |
| ATOM | 893 | CB | PHE | L | 118 | 18.150 | 6.305 | 23.761 | 1.00 | 13.52 | L |
| ATOM | 894 | CG | PHE | L | 118 | 19.487 | 5.708 | 23.414 | 1.00 | 12.88 | L |
| ATOM | 895 | CD1 | PHE | L | 118 | 19.583 | 4.672 | 22.493 | 1.00 | 14.61 | L |
| ATOM | 896 | CD2 | PHE | L | 118 | 20.653 | 6.200 | 23.989 | 1.00 | 12.50 | L |
| ATOM | 897 | CE1 | PHE | L | 118 | 20.824 | 4.134 | 22.143 | 1.00 | 15.97 | L |
| ATOM | 898 | CE2 | PHE | L | 118 | 21.896 | 5.672 | 23.648 | 1.00 | 15.65 | L |
| ATOM | 899 | CZ | PHE | L | 118 | 21.984 | 4.637 | 22.722 | 1.00 | 14.42 | L |
| ATOM | 900 | C | PHE | L | 118 | 16.089 | 6.303 | 25.118 | 1.00 | 17.37 | L |
| ATOM | 901 | O | PHE | L | 118 | 15.189 | 6.123 | 24.308 | 1.00 | 15.85 | L |
| ATOM | 902 | N | PRO | L | 119 | 15.925 | 7.071 | 26.200 | 1.00 | 18.64 | L |
| ATOM | 903 | CD | PRO | L | 119 | 16.758 | 7.069 | 27.414 | 1.00 | 18.51 | L |
| ATOM | 904 | CA | PRO | L | 119 | 14.655 | 7.768 | 26.433 | 1.00 | 18.81 | L |
| ATOM | 905 | CB | PRO | L | 119 | 14.748 | 8.182 | 27.902 | 1.00 | 19.12 | L |
| ATOM | 906 | CG | PRO | L | 119 | 15.719 | 7.181 | 28.495 | 1.00 | 22.45 | L |
| ATOM | 907 | C | PRO | L | 119 | 14.617 | 8.991 | 25.511 | 1.00 | 18.41 | L |
| ATOM | 908 | O | PRO | L | 119 | 15.602 | 9.303 | 24.853 | 1.00 | 17.86 | L |
| ATOM | 909 | N | PRO | L | 120 | 13.476 | 9.687 | 25.436 | 1.00 | 18.60 | L |
| ATOM | 910 | CD | PRO | L | 120 | 12.146 | 9.430 | 26.018 | 1.00 | 20.08 | L |
| ATOM | 911 | CA | PRO | L | 120 | 13.453 | 10.863 | 24.563 | 1.00 | 18.01 | L |
| ATOM | 912 | CB | PRO | L | 120 | 11.968 | 11.196 | 24.476 | 1.00 | 17.63 | L |
| ATOM | 913 | CG | PRO | L | 120 | 11.446 | 10.754 | 25.813 | 1.00 | 18.78 | L |
| ATOM | 914 | C | PRO | L | 120 | 14.263 | 11.987 | 25.213 | 1.00 | 18.07 | L |
| ATOM | 915 | O | PRO | L | 120 | 14.332 | 12.075 | 26.435 | 1.00 | 16.65 | L |
| ATOM | 916 | N | SER | L | 121 | 14.889 | 12.828 | 24.403 | 1.00 | 17.00 | L |
| ATOM | 917 | CA | SER | L | 121 | 15.663 | 13.948 | 24.935 | 1.00 | 19.17 | L |
| ATOM | 918 | CB | SER | L | 121 | 16.534 | 14.570 | 23.844 | 1.00 | 18.67 | L |
| ATOM | 919 | OG | SER | L | 121 | 15.728 | 15.169 | 22.832 | 1.00 | 20.17 | L |
| ATOM | 920 | C | SER | L | 121 | 14.689 | 15.006 | 25.434 | 1.00 | 20.98 | L |
| ATOM | 921 | O | SER | L | 121 | 13.547 | 15.071 | 24.977 | 1.00 | 19.84 | L |
| ATOM | 922 | N | ASP | L | 122 | 15.133 | 15.833 | 26.373 | 1.00 | 23.11 | L |
| ATOM | 923 | CA | ASP | L | 122 | 14.281 | 16.895 | 26.885 | 1.00 | 25.74 | L |
| ATOM | 924 | CB | ASP | L | 122 | 14.960 | 17.593 | 28.064 | 1.00 | 30.84 | L |
| ATOM | 925 | CG | ASP | L | 122 | 14.826 | 16.809 | 29.352 | 1.00 | 35.79 | L |
| ATOM | 926 | OD1 | ASP | L | 122 | 15.622 | 17.060 | 30.281 | 1.00 | 40.80 | L |
| ATOM | 927 | OD2 | ASP | L | 122 | 13.920 | 15.947 | 29.438 | 1.00 | 37.74 | L |
| ATOM | 928 | C | ASP | L | 122 | 14.003 | 17.891 | 25.763 | 1.00 | 24.67 | L |
| ATOM | 929 | O | ASP | L | 122 | 12.955 | 18.534 | 25.733 | 1.00 | 26.01 | L |
| ATOM | 930 | N | GLU | L | 123 | 14.943 | 18.005 | 24.832 | 1.00 | 23.96 | L |
| ATOM | 931 | CA | GLU | L | 123 | 14.769 | 18.911 | 23.710 | 1.00 | 24.25 | L |
| ATOM | 932 | CB | GLU | L | 123 | 16.011 | 18.928 | 22.824 | 1.00 | 26.90 | L |
| ATOM | 933 | CG | GLU | L | 123 | 15.859 | 19.851 | 21.623 | 1.00 | 33.92 | L |
| ATOM | 934 | CD | GLU | L | 123 | 17.110 | 19.946 | 20.765 | 1.00 | 39.23 | L |
| ATOM | 935 | OE1 | GLU | L | 123 | 17.041 | 20.603 | 19.701 | 1.00 | 39.60 | L |
| ATOM | 936 | OE2 | GLU | L | 123 | 18.157 | 19.374 | 21.151 | 1.00 | 39.67 | L |
| ATOM | 937 | C | GLU | L | 123 | 13.553 | 18.511 | 22.883 | 1.00 | 23.82 | L |
| ATOM | 938 | O | GLU | L | 123 | 12.720 | 19.360 | 22.561 | 1.00 | 25.97 | L |
| ATOM | 939 | N | GLN | L | 124 | 13.443 | 17.228 | 22.541 | 1.00 | 19.86 | L |
| ATOM | 940 | CA | GLN | L | 124 | 12.303 | 16.755 | 21.752 | 1.00 | 20.28 | L |
| ATOM | 941 | CB | GLN | L | 124 | 12.508 | 15.301 | 21.293 | 1.00 | 16.53 | L |
| ATOM | 942 | CG | GLN | L | 124 | 11.289 | 14.708 | 20.582 | 1.00 | 17.68 | L |
| ATOM | 943 | CD | GLN | L | 124 | 11.468 | 13.251 | 20.184 | 1.00 | 14.94 | L |
| ATOM | 944 | OE1 | GLN | L | 124 | 12.027 | 12.452 | 20.936 | 1.00 | 16.31 | L |
| ATOM | 945 | NE2 | GLN | L | 124 | 10.975 | 12.897 | 19.006 | 1.00 | 16.58 | L |
| ATOM | 946 | C | GLN | L | 124 | 10.996 | 16.862 | 22.541 | 1.00 | 20.65 | L |
| ATOM | 947 | O | GLN | L | 124 | 9.951 | 17.206 | 21.985 | 1.00 | 18.60 | L |
| ATOM | 948 | N | LEU | L | 125 | 11.050 | 16.558 | 23.832 | 1.00 | 21.36 | L |
| ATOM | 949 | CA | LEU | L | 125 | 9.852 | 16.643 | 24.654 | 1.00 | 25.45 | L |
| ATOM | 950 | CB | LEU | L | 125 | 10.173 | 16.300 | 26.110 | 1.00 | 24.67 | L |
| ATOM | 951 | CG | LEU | L | 125 | 10.222 | 14.788 | 26.335 | 1.00 | 26.63 | L |
| ATOM | 952 | CD1 | LEU | L | 125 | 10.621 | 14.484 | 27.767 | 1.00 | 26.53 | L |
| ATOM | 953 | CD2 | LEU | L | 125 | 8.855 | 14.187 | 26.005 | 1.00 | 23.96 | L |
| ATOM | 954 | C | LEU | L | 125 | 9.227 | 18.026 | 24.563 | 1.00 | 27.70 | L |
| ATOM | 955 | O | LEU | L | 125 | 8.012 | 18.158 | 24.426 | 1.00 | 28.28 | L |
| ATOM | 956 | N | LYS | L | 126 | 10.064 | 19.056 | 24.621 | 1.00 | 30.62 | L |
| ATOM | 957 | CA | LYS | L | 126 | 9.588 | 20.432 | 24.535 | 1.00 | 33.56 | L |
| ATOM | 958 | CB | LYS | L | 126 | 10.766 | 21.405 | 24.633 | 1.00 | 36.28 | L |
| ATOM | 959 | CG | LYS | L | 126 | 11.215 | 21.707 | 26.053 | 1.00 | 39.19 | L |
| ATOM | 960 | CD | LYS | L | 126 | 12.660 | 22.189 | 26.078 | 1.00 | 41.83 | L |
| ATOM | 961 | CE | LYS | L | 126 | 12.885 | 23.358 | 25.133 | 1.00 | 42.88 | L |
| ATOM | 962 | NZ | LYS | L | 126 | 14.338 | 23.677 | 25.000 | 1.00 | 45.62 | L |
| ATOM | 963 | C | LYS | L | 126 | 8.820 | 20.703 | 23.249 | 1.00 | 33.51 | L |
| ATOM | 964 | O | LYS | L | 126 | 7.967 | 21.589 | 23.208 | 1.00 | 33.70 | L |
| ATOM | 965 | N | SER | L | 127 | 9.119 | 19.940 | 22.202 | 1.00 | 33.72 | L |
| ATOM | 966 | CA | SER | L | 127 | 8.444 | 20.126 | 20.923 | 1.00 | 33.22 | L |
| ATOM | 967 | CB | SER | L | 127 | 9.348 | 19.683 | 19.765 | 1.00 | 33.54 | L |
| ATOM | 968 | OG | SER | L | 127 | 9.484 | 18.276 | 19.712 | 1.00 | 36.34 | L |
| ATOM | 969 | C | SER | L | 127 | 7.106 | 19.390 | 20.849 | 1.00 | 32.36 | L |
| ATOM | 970 | O | SER | L | 127 | 6.387 | 19.509 | 19.859 | 1.00 | 34.29 | L |
| ATOM | 971 | N | GLY | L | 128 | 6.779 | 18.623 | 21.884 | 1.00 | 30.85 | L |
| ATOM | 972 | CA | GLY | L | 128 | 5.504 | 17.917 | 21.897 | 1.00 | 30.24 | L |
| ATOM | 973 | C | GLY | L | 128 | 5.463 | 16.459 | 21.468 | 1.00 | 29.39 | L |
| ATOM | 974 | O | GLY | L | 128 | 4.391 | 15.854 | 21.452 | 1.00 | 29.51 | L |
| ATOM | 975 | N | THR | L | 129 | 6.614 | 15.882 | 21.135 | 1.00 | 28.50 | L |
| ATOM | 976 | CA | THR | L | 129 | 6.668 | 14.486 | 20.707 | 1.00 | 26.45 | L |
| ATOM | 977 | CB | THR | L | 129 | 7.017 | 14.397 | 19.197 | 1.00 | 28.88 | L |
| ATOM | 978 | OG1 | THR | L | 129 | 5.961 | 14.990 | 18.430 | 1.00 | 31.47 | L |
| ATOM | 979 | CG2 | THR | L | 129 | 7.190 | 12.955 | 18.758 | 1.00 | 29.75 | L |
| ATOM | 980 | C | THR | L | 129 | 7.702 | 13.712 | 21.526 | 1.00 | 25.33 | L |
| ATOM | 981 | O | THR | L | 129 | 8.582 | 14.306 | 22.144 | 1.00 | 25.39 | L |
| ATOM | 982 | N | ALA | L | 130 | 7.588 | 12.388 | 21.541 | 1.00 | 20.90 | L |
| ATOM | 983 | CA | ALA | L | 130 | 8.526 | 11.566 | 22.288 | 1.00 | 19.57 | L |
| ATOM | 984 | CB | ALA | L | 130 | 7.907 | 11.138 | 23.611 | 1.00 | 19.93 | L |
| ATOM | 985 | C | ALA | L | 130 | 8.969 | 10.339 | 21.495 | 1.00 | 19.60 | L |
| ATOM | 986 | O | ALA | L | 130 | 8.157 | 9.475 | 21.151 | 1.00 | 17.30 | L |
| ATOM | 987 | N | SER | L | 131 | 10.264 | 10.278 | 21.199 | 1.00 | 16.04 | L |
| ATOM | 988 | CA | SER | L | 131 | 10.820 | 9.154 | 20.460 | 1.00 | 17.42 | L |
| ATOM | 989 | CB | SER | L | 131 | 11.603 | 9.637 | 19.246 | 1.00 | 15.64 | L |
| ATOM | 990 | OG | SER | L | 131 | 10.767 | 10.338 | 18.346 | 1.00 | 18.71 | L |
| ATOM | 991 | C | SER | L | 131 | 11.737 | 8.335 | 21.359 | 1.00 | 17.74 | L |
| ATOM | 992 | O | SER | L | 131 | 12.681 | 8.859 | 21.948 | 1.00 | 18.10 | L |
| ATOM | 993 | N | VAL | L | 132 | 11.442 | 7.047 | 21.466 | 1.00 | 15.94 | L |
| ATOM | 994 | CA | VAL | L | 132 | 12.231 | 6.144 | 22.288 | 1.00 | 15.94 | L |
| ATOM | 995 | CB | VAL | L | 132 | 11.330 | 5.297 | 23.206 | 1.00 | 15.29 | L |
| ATOM | 996 | CG1 | VAL | L | 132 | 12.182 | 4.538 | 24.213 | 1.00 | 14.95 | L |
| ATOM | 997 | CG2 | VAL | L | 132 | 10.325 | 6.194 | 23.917 | 1.00 | 17.50 | L |
| ATOM | 998 | C | VAL | L | 132 | 12.968 | 5.234 | 21.325 | 1.00 | 15.15 | L |
| ATOM | 999 | O | VAL | L | 132 | 12.348 | 4.593 | 20.477 | 1.00 | 16.27 | L |
| ATOM | 1000 | N | VAL | L | 133 | 14.287 | 5.180 | 21.447 | 1.00 | 13.28 | L |
| ATOM | 1001 | CA | VAL | L | 133 | 15.080 | 4.358 | 20.543 | 1.00 | 15.24 | L |
| ATOM | 1002 | CB | VAL | L | 133 | 16.216 | 5.200 | 19.895 | 1.00 | 15.01 | L |
| ATOM | 1003 | CG1 | VAL | L | 133 | 17.016 | 4.350 | 18.920 | 1.00 | 16.00 | L |
| ATOM | 1004 | CG2 | VAL | L | 133 | 15.623 | 6.405 | 19.176 | 1.00 | 13.71 | L |
| ATOM | 1005 | C | VAL | L | 133 | 15.696 | 3.130 | 21.210 | 1.00 | 16.12 | L |
| ATOM | 1006 | O | VAL | L | 133 | 16.140 | 3.179 | 22.358 | 1.00 | 15.13 | L |
| ATOM | 1007 | N | CYS | L | 134 | 15.711 | 2.029 | 20.472 | 1.00 | 14.28 | L |
| ATOM | 1008 | CA | CYS | L | 134 | 16.289 | 0.790 | 20.949 | 1.00 | 17.10 | L |
| ATOM | 1009 | C | CYS | L | 134 | 17.322 | 0.351 | 19.932 | 1.00 | 17.14 | L |
| ATOM | 1010 | O | CYS | L | 134 | 17.018 | 0.246 | 18.746 | 1.00 | 17.36 | L |
| ATOM | 1011 | CB | CYS | L | 134 | 15.233 | −0.303 | 21.070 | 1.00 | 20.24 | L |
| ATOM | 1012 | SG | CYS | L | 134 | 15.891 | −1.817 | 21.835 | 1.00 | 24.74 | L |
| ATOM | 1013 | N | LEU | L | 135 | 18.537 | 0.088 | 20.402 | 1.00 | 16.72 | L |
| ATOM | 1014 | CA | LEU | L | 135 | 19.616 | −0.338 | 19.522 | 1.00 | 16.54 | L |
| ATOM | 1015 | CB | LEU | L | 135 | 20.803 | 0.616 | 19.658 | 1.00 | 15.42 | L |
| ATOM | 1016 | CG | LEU | L | 135 | 22.152 | 0.097 | 19.147 | 1.00 | 16.09 | L |
| ATOM | 1017 | CD1 | LEU | L | 135 | 22.108 | −0.100 | 17.643 | 1.00 | 14.03 | L |
| ATOM | 1018 | CD2 | LEU | L | 135 | 23.250 | 1.088 | 19.517 | 1.00 | 17.08 | L |
| ATOM | 1019 | C | LEU | L | 135 | 20.090 | −1.763 | 19.806 | 1.00 | 16.14 | L |
| ATOM | 1020 | O | LEU | L | 135 | 20.390 | −2.111 | 20.944 | 1.00 | 16.25 | L |
| ATOM | 1021 | N | LEU | L | 136 | 20.132 | −2.583 | 18.765 | 1.00 | 13.78 | L |
| ATOM | 1022 | CA | LEU | L | 136 | 20.625 | −3.950 | 18.874 | 1.00 | 13.48 | L |
| ATOM | 1023 | CB | LEU | L | 136 | 19.675 | −4.939 | 18.192 | 1.00 | 9.63 | L |
| ATOM | 1024 | CG | LEU | L | 136 | 18.413 | −5.292 | 18.978 | 1.00 | 12.24 | L |
| ATOM | 1025 | CD1 | LEU | L | 136 | 17.594 | −4.030 | 19.286 | 1.00 | 11.59 | L |
| ATOM | 1026 | CD2 | LEU | L | 136 | 17.595 | −6.287 | 18.170 | 1.00 | 11.34 | L |
| ATOM | 1027 | C | LEU | L | 136 | 21.945 | −3.862 | 18.123 | 1.00 | 12.56 | L |
| ATOM | 1028 | O | LEU | L | 136 | 21.967 | −3.698 | 16.909 | 1.00 | 11.82 | L |
| ATOM | 1029 | N | ASN | L | 137 | 23.047 | −3.973 | 18.851 | 1.00 | 14.77 | L |
| ATOM | 1030 | CA | ASN | L | 137 | 24.355 | −3.805 | 18.246 | 1.00 | 12.54 | L |
| ATOM | 1031 | CB | ASN | L | 137 | 25.152 | −2.811 | 19.091 | 1.00 | 13.30 | L |
| ATOM | 1032 | CG | ASN | L | 137 | 26.231 | −2.108 | 18.297 | 1.00 | 16.58 | L |
| ATOM | 1033 | OD1 | ASN | L | 137 | 25.942 | −1.391 | 17.339 | 1.00 | 19.74 | L |
| ATOM | 1034 | ND2 | ASN | L | 137 | 27.479 | −2.312 | 18.686 | 1.00 | 17.03 | L |
| ATOM | 1035 | C | ASN | L | 137 | 25.217 | −5.031 | 17.972 | 1.00 | 14.59 | L |
| ATOM | 1036 | O | ASN | L | 137 | 25.370 | −5.907 | 18.818 | 1.00 | 11.08 | L |
| ATOM | 1037 | N | ASN | L | 138 | 25.770 | −5.064 | 16.763 | 1.00 | 13.98 | L |
| ATOM | 1038 | CA | ASN | L | 138 | 26.682 | −6.107 | 16.308 | 1.00 | 16.43 | L |
| ATOM | 1039 | CB | ASN | L | 138 | 28.064 | −5.846 | 16.918 | 1.00 | 17.39 | L |
| ATOM | 1040 | CG | ASN | L | 138 | 28.668 | −4.509 | 16.468 | 1.00 | 20.78 | L |
| ATOM | 1041 | OD1 | ASN | L | 138 | 27.970 | −3.625 | 15.972 | 1.00 | 15.92 | L |
| ATOM | 1042 | ND2 | ASN | L | 138 | 29.974 | −4.362 | 16.659 | 1.00 | 22.02 | L |
| ATOM | 1043 | C | ASN | L | 138 | 26.276 | −7.568 | 16.557 | 1.00 | 16.20 | L |
| ATOM | 1044 | O | ASN | L | 138 | 26.953 | −8.293 | 17.284 | 1.00 | 13.80 | L |
| ATOM | 1045 | N | PHE | L | 139 | 25.183 | −8.001 | 15.939 | 1.00 | 16.21 | L |
| ATOM | 1046 | CA | PHE | L | 139 | 24.727 | −9.374 | 16.094 | 1.00 | 15.34 | L |
| ATOM | 1047 | CB | PHE | L | 139 | 23.269 | −9.415 | 16.568 | 1.00 | 14.26 | L |
| ATOM | 1048 | CG | PHE | L | 139 | 22.323 | −8.628 | 15.706 | 1.00 | 14.43 | L |
| ATOM | 1049 | CD1 | PHE | L | 139 | 22.073 | −7.282 | 15.973 | 1.00 | 14.21 | L |
| ATOM | 1050 | CD2 | PHE | L | 139 | 21.674 | −9.229 | 14.630 | 1.00 | 13.14 | L |
| ATOM | 1051 | CE1 | PHE | L | 139 | 21.187 | −6.548 | 15.183 | 1.00 | 15.78 | L |
| ATOM | 1052 | CE2 | PHE | L | 139 | 20.788 | −8.506 | 13.834 | 1.00 | 15.16 | L |
| ATOM | 1053 | CZ | PHE | L | 139 | 20.541 | −7.163 | 14.108 | 1.00 | 14.80 | L |
| ATOM | 1054 | C | PHE | L | 139 | 24.846 | −10.166 | 14.793 | 1.00 | 16.32 | L |
| ATOM | 1055 | O | PHE | L | 139 | 25.028 | −9.594 | 13.718 | 1.00 | 14.73 | L |
| ATOM | 1056 | N | TYR | L | 140 | 24.763 | −11.489 | 14.916 | 1.00 | 15.17 | L |
| ATOM | 1057 | CA | TYR | L | 140 | 24.801 | −12.401 | 13.777 | 1.00 | 17.20 | L |
| ATOM | 1058 | CB | TYR | L | 140 | 26.233 | −12.621 | 13.266 | 1.00 | 15.00 | L |
| ATOM | 1059 | CG | TYR | L | 140 | 26.244 | −13.459 | 12.008 | 1.00 | 16.67 | L |
| ATOM | 1060 | CD1 | TYR | L | 140 | 26.169 | −14.856 | 12.076 | 1.00 | 14.25 | L |
| ATOM | 1061 | CE1 | TYR | L | 140 | 26.014 | −15.627 | 10.931 | 1.00 | 13.54 | L |
| ATOM | 1062 | CD2 | TYR | L | 140 | 26.181 | −12.857 | 10.752 | 1.00 | 10.85 | L |
| ATOM | 1063 | CE2 | TYR | L | 140 | 26.026 | −13.616 | 9.599 | 1.00 | 14.34 | L |
| ATOM | 1064 | CZ | TYR | L | 140 | 25.935 | −15.004 | 9.694 | 1.00 | 14.42 | L |
| ATOM | 1065 | OH | TYR | L | 140 | 25.709 | −15.755 | 8.562 | 1.00 | 12.60 | L |
| ATOM | 1066 | C | TYR | L | 140 | 24.205 | −13.734 | 14.233 | 1.00 | 16.42 | L |
| ATOM | 1067 | O | TYR | L | 140 | 24.496 | −14.197 | 15.334 | 1.00 | 16.55 | L |
| ATOM | 1068 | N | PRO | L | 141 | 23.359 | −14.370 | 13.397 | 1.00 | 16.75 | L |
| ATOM | 1069 | CD | PRO | L | 141 | 22.613 | −15.554 | 13.870 | 1.00 | 15.71 | L |
| ATOM | 1070 | CA | PRO | L | 141 | 22.901 | −13.993 | 12.057 | 1.00 | 15.91 | L |
| ATOM | 1071 | CB | PRO | L | 141 | 22.187 | −15.251 | 11.586 | 1.00 | 15.43 | L |
| ATOM | 1072 | CG | PRO | L | 141 | 21.493 | −15.674 | 12.846 | 1.00 | 14.95 | L |
| ATOM | 1073 | C | PRO | L | 141 | 21.976 | −12.776 | 12.059 | 1.00 | 14.55 | L |
| ATOM | 1074 | O | PRO | L | 141 | 21.594 | −12.281 | 13.112 | 1.00 | 16.24 | L |
| ATOM | 1075 | N | ARG | L | 142 | 21.603 | −12.321 | 10.868 | 1.00 | 15.92 | L |
| ATOM | 1076 | CA | ARG | L | 142 | 20.765 | −11.136 | 10.714 | 1.00 | 17.12 | L |
| ATOM | 1077 | CB | ARG | L | 142 | 20.634 | −10.781 | 9.228 | 1.00 | 19.78 | L |
| ATOM | 1078 | CG | ARG | L | 142 | 19.791 | −9.535 | 8.985 | 1.00 | 23.11 | L |
| ATOM | 1079 | CD | ARG | L | 142 | 19.716 | −9.144 | 7.509 | 1.00 | 29.07 | L |
| ATOM | 1080 | NE | ARG | L | 142 | 18.433 | −8.505 | 7.219 | 1.00 | 31.39 | L |
| ATOM | 1081 | CZ | ARG | L | 142 | 18.278 | −7.229 | 6.891 | 1.00 | 36.35 | L |
| ATOM | 1082 | NH1 | ARG | L | 142 | 19.322 | −6.505 | 6.497 | 1.00 | 40.49 | L |
| ATOM | 1083 | NH2 | ARG | L | 142 | 17.073 | −6.673 | 6.972 | 1.00 | 35.08 | L |
| ATOM | 1084 | C | ARG | L | 142 | 19.376 | −11.174 | 11.349 | 1.00 | 18.16 | L |
| ATOM | 1085 | O | ARG | L | 142 | 18.846 | −10.129 | 11.743 | 1.00 | 16.16 | L |
| ATOM | 1086 | N | GLU | L | 143 | 18.785 | −12.362 | 11.452 | 1.00 | 19.16 | L |
| ATOM | 1087 | CA | GLU | L | 143 | 17.458 | −12.491 | 12.043 | 1.00 | 20.63 | L |
| ATOM | 1088 | CB | GLU | L | 143 | 16.977 | −13.948 | 11.979 | 1.00 | 23.34 | L |
| ATOM | 1089 | CG | GLU | L | 143 | 16.625 | −14.444 | 10.579 | 1.00 | 28.64 | L |
| ATOM | 1090 | CD | GLU | L | 143 | 17.789 | −14.390 | 9.608 | 1.00 | 30.60 | L |
| ATOM | 1091 | OE1 | GLU | L | 143 | 18.914 | −14.785 | 9.990 | 1.00 | 31.02 | L |
| ATOM | 1092 | OE2 | GLU | L | 143 | 17.571 | −13.964 | 8.453 | 1.00 | 32.44 | L |
| ATOM | 1093 | C | GLU | L | 143 | 17.407 | −12.015 | 13.492 | 1.00 | 20.43 | L |
| ATOM | 1094 | O | GLU | L | 143 | 18.123 | −12.520 | 14.347 | 1.00 | 21.52 | L |
| ATOM | 1095 | N | ALA | L | 144 | 16.552 | −11.038 | 13.760 | 1.00 | 19.39 | L |
| ATOM | 1096 | CA | ALA | L | 144 | 16.388 | −10.503 | 15.107 | 1.00 | 21.56 | L |
| ATOM | 1097 | CB | ALA | L | 144 | 17.400 | −9.403 | 15.373 | 1.00 | 21.99 | L |
| ATOM | 1098 | C | ALA | L | 144 | 14.979 | −9.949 | 15.200 | 1.00 | 21.08 | L |
| ATOM | 1099 | O | ALA | L | 144 | 14.379 | −9.615 | 14.184 | 1.00 | 21.32 | L |
| ATOM | 1100 | N | LYS | L | 145 | 14.447 | −9.861 | 16.411 | 1.00 | 19.33 | L |
| ATOM | 1101 | CA | LYS | L | 145 | 13.105 | −9.341 | 16.592 | 1.00 | 20.40 | L |
| ATOM | 1102 | CB | LYS | L | 145 | 12.099 | −10.486 | 16.767 | 1.00 | 24.22 | L |
| ATOM | 1103 | CG | LYS | L | 145 | 10.652 | −10.022 | 16.655 | 1.00 | 29.01 | L |
| ATOM | 1104 | CD | LYS | L | 145 | 9.876 | −10.220 | 17.941 | 1.00 | 33.29 | L |
| ATOM | 1105 | CE | LYS | L | 145 | 9.121 | −11.540 | 17.936 | 1.00 | 36.48 | L |
| ATOM | 1106 | NZ | LYS | L | 145 | 8.111 | −11.594 | 16.836 | 1.00 | 38.85 | L |
| ATOM | 1107 | C | LYS | L | 145 | 13.049 | −8.426 | 17.803 | 1.00 | 19.42 | L |
| ATOM | 1108 | O | LYS | L | 145 | 13.602 | −8.735 | 18.855 | 1.00 | 19.22 | L |
| ATOM | 1109 | N | VAL | L | 146 | 12.368 | −7.301 | 17.645 | 1.00 | 19.42 | L |
| ATOM | 1110 | CA | VAL | L | 146 | 12.232 | −6.328 | 18.714 | 1.00 | 19.31 | L |
| ATOM | 1111 | CB | VAL | L | 146 | 12.863 | −4.969 | 18.313 | 1.00 | 18.27 | L |
| ATOM | 1112 | CG1 | VAL | L | 146 | 12.673 | −3.954 | 19.423 | 1.00 | 20.69 | L |
| ATOM | 1113 | CG2 | VAL | L | 146 | 14.336 | −5.150 | 18.007 | 1.00 | 18.47 | L |
| ATOM | 1114 | C | VAL | L | 146 | 10.763 | −6.099 | 19.022 | 1.00 | 18.65 | L |
| ATOM | 1115 | O | VAL | L | 146 | 9.957 | −5.914 | 18.116 | 1.00 | 20.53 | L |
| ATOM | 1116 | N | GLN | L | 147 | 10.421 | −6.125 | 20.305 | 1.00 | 17.62 | L |
| ATOM | 1117 | CA | GLN | L | 147 | 9.055 | −5.884 | 20.740 | 1.00 | 17.65 | L |
| ATOM | 1118 | CB | GLN | L | 147 | 8.453 | −7.134 | 21.397 | 1.00 | 20.72 | L |
| ATOM | 1119 | CG | GLN | L | 147 | 7.931 | −8.167 | 20.410 | 1.00 | 23.13 | L |
| ATOM | 1120 | CD | GLN | L | 147 | 7.180 | −9.311 | 21.091 | 1.00 | 27.74 | L |
| ATOM | 1121 | OE1 | GLN | L | 147 | 6.188 | −9.817 | 20.561 | 1.00 | 27.62 | L |
| ATOM | 1122 | NE2 | GLN | L | 147 | 7.660 | −9.729 | 22.261 | 1.00 | 24.04 | L |
| ATOM | 1123 | C | GLN | L | 147 | 9.074 | −4.747 | 21.743 | 1.00 | 18.50 | L |
| ATOM | 1124 | O | GLN | L | 147 | 9.888 | −4.733 | 22.672 | 1.00 | 17.74 | L |
| ATOM | 1125 | N | TRP | L | 148 | 8.194 | −3.778 | 21.542 | 1.00 | 18.12 | L |
| ATOM | 1126 | CA | TRP | L | 148 | 8.106 | −2.651 | 22.450 | 1.00 | 17.11 | L |
| ATOM | 1127 | CB | TRP | L | 148 | 7.832 | −1.359 | 21.684 | 1.00 | 16.94 | L |
| ATOM | 1128 | CG | TRP | L | 148 | 9.034 | −0.788 | 21.008 | 1.00 | 17.78 | L |
| ATOM | 1129 | CD2 | TRP | L | 148 | 10.077 | −0.019 | 21.623 | 1.00 | 16.77 | L |
| ATOM | 1130 | CE2 | TRP | L | 148 | 10.968 | 0.367 | 20.599 | 1.00 | 17.75 | L |
| ATOM | 1131 | CE3 | TRP | L | 148 | 10.340 | 0.385 | 22.941 | 1.00 | 17.55 | L |
| ATOM | 1132 | CD1 | TRP | L | 148 | 9.332 | −0.845 | 19.679 | 1.00 | 17.08 | L |
| ATOM | 1133 | NE1 | TRP | L | 148 | 10.490 | −0.151 | 19.423 | 1.00 | 19.01 | L |
| ATOM | 1134 | CZ2 | TRP | L | 148 | 12.109 | 1.143 | 20.847 | 1.00 | 16.23 | L |
| ATOM | 1135 | CZ3 | TRP | L | 148 | 11.477 | 1.157 | 23.191 | 1.00 | 17.38 | L |
| ATOM | 1136 | CH2 | TRP | L | 148 | 12.346 | 1.529 | 22.144 | 1.00 | 15.69 | L |
| ATOM | 1137 | C | TRP | L | 148 | 6.988 | −2.889 | 23.455 | 1.00 | 16.79 | L |
| ATOM | 1138 | O | TRP | L | 148 | 5.873 | −3.262 | 23.091 | 1.00 | 16.55 | L |
| ATOM | 1139 | N | LYS | L | 149 | 7.296 | −2.696 | 24.728 | 1.00 | 16.94 | L |
| ATOM | 1140 | CA | LYS | L | 149 | 6.296 | −2.869 | 25.766 | 1.00 | 17.23 | L |
| ATOM | 1141 | CB | LYS | L | 149 | 6.563 | −4.149 | 26.568 | 1.00 | 17.53 | L |
| ATOM | 1142 | CG | LYS | L | 149 | 6.305 | −5.424 | 25.760 | 1.00 | 18.77 | L |
| ATOM | 1143 | CD | LYS | L | 149 | 6.695 | −6.695 | 26.503 | 1.00 | 22.14 | L |
| ATOM | 1144 | CE | LYS | L | 149 | 6.276 | −7.936 | 25.707 | 1.00 | 22.20 | L |
| ATOM | 1145 | NZ | LYS | L | 149 | 6.708 | −9.224 | 26.330 | 1.00 | 23.55 | L |
| ATOM | 1146 | C | LYS | L | 149 | 6.310 | −1.649 | 26.662 | 1.00 | 18.85 | L |
| ATOM | 1147 | O | LYS | L | 149 | 7.361 | −1.233 | 27.146 | 1.00 | 21.46 | L |
| ATOM | 1148 | N | VAL | L | 150 | 5.134 | −1.056 | 26.835 | 1.00 | 18.34 | L |
| ATOM | 1149 | CA | VAL | L | 150 | 4.960 | 0.113 | 27.680 | 1.00 | 19.06 | L |
| ATOM | 1150 | CB | VAL | L | 150 | 4.290 | 1.251 | 26.908 | 1.00 | 18.50 | L |
| ATOM | 1151 | CG1 | VAL | L | 150 | 4.067 | 2.429 | 27.815 | 1.00 | 17.72 | L |
| ATOM | 1152 | CG2 | VAL | L | 150 | 5.163 | 1.645 | 25.722 | 1.00 | 17.70 | L |
| ATOM | 1153 | C | VAL | L | 150 | 4.058 | −0.340 | 28.820 | 1.00 | 21.24 | L |
| ATOM | 1154 | O | VAL | L | 150 | 2.894 | −0.677 | 28.601 | 1.00 | 20.72 | L |
| ATOM | 1155 | N | ASP | L | 151 | 4.601 | −0.345 | 30.033 | 1.00 | 20.68 | L |
| ATOM | 1156 | CA | ASP | L | 151 | 3.857 | −0.813 | 31.196 | 1.00 | 23.34 | L |
| ATOM | 1157 | CB | ASP | L | 151 | 2.630 | 0.063 | 31.468 | 1.00 | 23.74 | L |
| ATOM | 1158 | CG | ASP | L | 151 | 3.006 | 1.410 | 32.066 | 1.00 | 25.47 | L |
| ATOM | 1159 | OD1 | ASP | L | 151 | 4.020 | 1.460 | 32.787 | 1.00 | 20.82 | L |
| ATOM | 1160 | OD2 | ASP | L | 151 | 2.290 | 2.406 | 31.828 | 1.00 | 25.93 | L |
| ATOM | 1161 | C | ASP | L | 151 | 3.442 | −2.246 | 30.903 | 1.00 | 22.88 | L |
| ATOM | 1162 | O | ASP | L | 151 | 2.360 | −2.692 | 31.271 | 1.00 | 22.56 | L |
| ATOM | 1163 | N | ASN | L | 152 | 4.330 | −2.947 | 30.204 | 1.00 | 21.80 | L |
| ATOM | 1164 | CA | ASN | L | 152 | 4.140 | −4.339 | 29.830 | 1.00 | 22.35 | L |
| ATOM | 1165 | CB | ASN | L | 152 | 3.841 | −5.171 | 31.075 | 1.00 | 21.87 | L |
| ATOM | 1166 | CG | ASN | L | 152 | 4.101 | −6.645 | 30.857 | 1.00 | 23.72 | L |
| ATOM | 1167 | OD1 | ASN | L | 152 | 5.151 | −7.033 | 30.339 | 1.00 | 22.06 | L |
| ATOM | 1168 | ND2 | ASN | L | 152 | 3.149 | −7.477 | 31.254 | 1.00 | 23.78 | L |
| ATOM | 1169 | C | ASN | L | 152 | 3.084 | −4.608 | 28.754 | 1.00 | 21.57 | L |
| ATOM | 1170 | O | ASN | L | 152 | 2.803 | −5.762 | 28.442 | 1.00 | 22.19 | L |
| ATOM | 1171 | N | ALA | L | 153 | 2.505 | −3.554 | 28.181 | 1.00 | 21.13 | L |
| ATOM | 1172 | CA | ALA | L | 153 | 1.502 | −3.724 | 27.123 | 1.00 | 20.13 | L |
| ATOM | 1173 | CB | ALA | L | 153 | 0.455 | −2.606 | 27.200 | 1.00 | 16.26 | L |
| ATOM | 1174 | C | ALA | L | 153 | 2.189 | −3.707 | 25.750 | 1.00 | 18.99 | L |
| ATOM | 1175 | O | ALA | L | 153 | 2.876 | −2.743 | 25.408 | 1.00 | 17.98 | L |
| ATOM | 1176 | N | LEU | L | 154 | 2.002 | −4.769 | 24.971 | 1.00 | 19.86 | L |
| ATOM | 1177 | CA | LEU | L | 154 | 2.614 | −4.869 | 23.639 | 1.00 | 21.14 | L |
| ATOM | 1178 | CB | LEU | L | 154 | 2.233 | −6.191 | 22.960 | 1.00 | 22.05 | L |
| ATOM | 1179 | CG | LEU | L | 154 | 3.366 | −6.946 | 22.250 | 1.00 | 27.94 | L |
| ATOM | 1180 | CD1 | LEU | L | 154 | 2.766 | −8.013 | 21.333 | 1.00 | 26.67 | L |
| ATOM | 1181 | CD2 | LEU | L | 154 | 4.230 | −5.987 | 21.447 | 1.00 | 22.78 | L |
| ATOM | 1182 | C | LEU | L | 154 | 2.190 | −3.715 | 22.738 | 1.00 | 19.77 | L |
| ATOM | 1183 | O | LEU | L | 154 | 1.001 | −3.437 | 22.591 | 1.00 | 21.09 | L |
| ATOM | 1184 | N | GLN | L | 155 | 3.168 | −3.058 | 22.125 | 1.00 | 16.93 | L |
| ATOM | 1185 | CA | GLN | L | 155 | 2.907 | −1.924 | 21.244 | 1.00 | 18.30 | L |
| ATOM | 1186 | CB | GLN | L | 155 | 4.033 | −0.890 | 21.371 | 1.00 | 15.27 | L |
| ATOM | 1187 | CG | GLN | L | 155 | 4.187 | −0.293 | 22.755 | 1.00 | 16.30 | L |
| ATOM | 1188 | CD | GLN | L | 155 | 2.940 | 0.412 | 23.216 | 1.00 | 14.29 | L |
| ATOM | 1189 | OE1 | GLN | L | 155 | 2.596 | 1.488 | 22.724 | 1.00 | 17.86 | L |
| ATOM | 1190 | NE2 | GLN | L | 155 | 2.241 | −0.199 | 24.161 | 1.00 | 14.86 | L |
| ATOM | 1191 | C | GLN | L | 155 | 2.779 | −2.322 | 19.776 | 1.00 | 18.62 | L |
| ATOM | 1192 | O | GLN | L | 155 | 3.494 | −3.197 | 19.300 | 1.00 | 20.18 | L |
| ATOM | 1193 | N | SER | L | 156 | 1.884 | −1.661 | 19.052 | 1.00 | 19.09 | L |
| ATOM | 1194 | CA | SER | L | 156 | 1.725 | −1.957 | 17.634 | 1.00 | 20.49 | L |
| ATOM | 1195 | CB | SER | L | 156 | 0.626 | −3.002 | 17.431 | 1.00 | 21.71 | L |
| ATOM | 1196 | OG | SER | L | 156 | 0.386 | −3.229 | 16.057 | 1.00 | 26.25 | L |
| ATOM | 1197 | C | SER | L | 156 | 1.400 | −0.706 | 16.831 | 1.00 | 20.15 | L |
| ATOM | 1198 | O | SER | L | 156 | 0.688 | 0.183 | 17.305 | 1.00 | 24.03 | L |
| ATOM | 1199 | N | GLY | L | 157 | 1.943 | −0.635 | 15.619 | 1.00 | 18.26 | L |
| ATOM | 1200 | CA | GLY | L | 157 | 1.685 | 0.495 | 14.748 | 1.00 | 17.56 | L |
| ATOM | 1201 | C | GLY | L | 157 | 2.346 | 1.811 | 15.112 | 1.00 | 19.08 | L |
| ATOM | 1202 | O | GLY | L | 157 | 2.109 | 2.824 | 14.453 | 1.00 | 18.92 | L |
| ATOM | 1203 | N | ASN | L | 158 | 3.169 | 1.821 | 16.153 | 1.00 | 18.41 | L |
| ATOM | 1204 | CA | ASN | L | 158 | 3.827 | 3.061 | 16.531 | 1.00 | 18.65 | L |
| ATOM | 1205 | CB | ASN | L | 158 | 3.273 | 3.575 | 17.868 | 1.00 | 15.36 | L |
| ATOM | 1206 | CG | ASN | L | 158 | 3.523 | 2.625 | 19.021 | 1.00 | 18.49 | L |
| ATOM | 1207 | OD1 | ASN | L | 158 | 4.144 | 1.574 | 18.864 | 1.00 | 19.14 | L |
| ATOM | 1208 | ND2 | ASN | L | 158 | 3.042 | 3.000 | 20.196 | 1.00 | 15.66 | L |
| ATOM | 1209 | C | ASN | L | 158 | 5.354 | 2.969 | 16.570 | 1.00 | 18.39 | L |
| ATOM | 1210 | O | ASN | L | 158 | 6.019 | 3.769 | 17.224 | 1.00 | 20.73 | L |
| ATOM | 1211 | N | SER | L | 159 | 5.914 | 2.002 | 15.852 | 1.00 | 18.10 | L |
| ATOM | 1212 | CA | SER | L | 159 | 7.362 | 1.864 | 15.814 | 1.00 | 18.10 | L |
| ATOM | 1213 | CB | SER | L | 159 | 7.830 | 0.859 | 16.871 | 1.00 | 16.87 | L |
| ATOM | 1214 | OG | SER | L | 159 | 7.501 | −0.470 | 16.510 | 1.00 | 17.98 | L |
| ATOM | 1215 | C | SER | L | 159 | 7.862 | 1.441 | 14.435 | 1.00 | 18.28 | L |
| ATOM | 1216 | O | SER | L | 159 | 7.131 | 0.837 | 13.662 | 1.00 | 18.92 | L |
| ATOM | 1217 | N | GLN | L | 160 | 9.109 | 1.778 | 14.125 | 1.00 | 17.96 | L |
| ATOM | 1218 | CA | GLN | L | 160 | 9.703 | 1.405 | 12.850 | 1.00 | 15.97 | L |
| ATOM | 1219 | CB | GLN | L | 160 | 9.579 | 2.545 | 11.832 | 1.00 | 17.56 | L |
| ATOM | 1220 | CG | GLN | L | 160 | 8.156 | 3.054 | 11.639 | 1.00 | 20.79 | L |
| ATOM | 1221 | CD | GLN | L | 160 | 8.035 | 4.056 | 10.506 | 1.00 | 23.44 | L |
| ATOM | 1222 | OE1 | GLN | L | 160 | 8.036 | 3.687 | 9.329 | 1.00 | 24.85 | L |
| ATOM | 1223 | NE2 | GLN | L | 160 | 7.941 | 5.334 | 10.855 | 1.00 | 25.69 | L |
| ATOM | 1224 | C | GLN | L | 160 | 11.172 | 1.077 | 13.094 | 1.00 | 15.65 | L |
| ATOM | 1225 | O | GLN | L | 160 | 11.775 | 1.551 | 14.059 | 1.00 | 14.81 | L |
| ATOM | 1226 | N | GLU | L | 161 | 11.743 | 0.273 | 12.209 | 1.00 | 13.95 | L |
| ATOM | 1227 | CA | GLU | L | 161 | 13.124 | −0.136 | 12.343 | 1.00 | 12.40 | L |
| ATOM | 1228 | CB | GLU | L | 161 | 13.190 | −1.491 | 13.056 | 1.00 | 13.86 | L |
| ATOM | 1229 | CG | GLU | L | 161 | 12.489 | −2.612 | 12.268 | 1.00 | 19.31 | L |
| ATOM | 1230 | CD | GLU | L | 161 | 12.553 | −3.970 | 12.953 | 1.00 | 22.06 | L |
| ATOM | 1231 | OE1 | GLU | L | 161 | 12.208 | −4.057 | 14.148 | 1.00 | 20.71 | L |
| ATOM | 1232 | OE2 | GLU | L | 161 | 12.941 | −4.957 | 12.293 | 1.00 | 24.92 | L |
| ATOM | 1233 | C | GLU | L | 161 | 13.808 | −0.269 | 10.990 | 1.00 | 13.80 | L |
| ATOM | 1234 | O | GLU | L | 161 | 13.157 | −0.324 | 9.939 | 1.00 | 12.61 | L |
| ATOM | 1235 | N | SER | L | 162 | 15.135 | −0.320 | 11.034 | 1.00 | 11.35 | L |
| ATOM | 1236 | CA | SER | L | 162 | 15.946 | −0.500 | 9.838 | 1.00 | 10.48 | L |
| ATOM | 1237 | CB | SER | L | 162 | 16.280 | 0.844 | 9.180 | 1.00 | 8.64 | L |
| ATOM | 1238 | OG | SER | L | 162 | 17.041 | 1.646 | 10.048 | 1.00 | 12.37 | L |
| ATOM | 1239 | C | SER | L | 162 | 17.210 | −1.201 | 10.319 | 1.00 | 9.34 | L |
| ATOM | 1240 | O | SER | L | 162 | 17.536 | −1.160 | 11.506 | 1.00 | 7.02 | L |
| ATOM | 1241 | N | VAL | L | 163 | 17.912 | −1.830 | 9.390 | 1.00 | 10.80 | L |
| ATOM | 1242 | CA | VAL | L | 163 | 19.109 | −2.599 | 9.691 | 1.00 | 10.51 | L |
| ATOM | 1243 | CB | VAL | L | 163 | 18.812 | −4.115 | 9.497 | 1.00 | 13.78 | L |
| ATOM | 1244 | CG1 | VAL | L | 163 | 20.008 | −4.968 | 9.911 | 1.00 | 14.19 | L |
| ATOM | 1245 | CG2 | VAL | L | 163 | 17.573 | −4.492 | 10.290 | 1.00 | 13.60 | L |
| ATOM | 1246 | C | VAL | L | 163 | 20.241 | −2.194 | 8.762 | 1.00 | 10.97 | L |
| ATOM | 1247 | O | VAL | L | 163 | 20.024 | −1.925 | 7.580 | 1.00 | 12.55 | L |
| ATOM | 1248 | N | THR | L | 164 | 21.449 | −2.146 | 9.304 | 1.00 | 12.01 | L |
| ATOM | 1249 | CA | THR | L | 164 | 22.621 | −1.789 | 8.519 | 1.00 | 13.85 | L |
| ATOM | 1250 | CB | THR | L | 164 | 23.837 | −1.509 | 9.424 | 1.00 | 16.12 | L |
| ATOM | 1251 | OG1 | THR | L | 164 | 24.046 | −2.636 | 10.288 | 1.00 | 15.55 | L |
| ATOM | 1252 | CG2 | THR | L | 164 | 23.623 | −0.256 | 10.270 | 1.00 | 14.42 | L |
| ATOM | 1253 | C | THR | L | 164 | 23.007 | −2.962 | 7.624 | 1.00 | 14.05 | L |
| ATOM | 1254 | O | THR | L | 164 | 22.548 | −4.083 | 7.828 | 1.00 | 14.03 | L |
| ATOM | 1255 | N | GLU | L | 165 | 23.846 | −2.689 | 6.631 | 1.00 | 13.70 | L |
| ATOM | 1256 | CA | GLU | L | 165 | 24.358 | −3.728 | 5.755 | 1.00 | 14.28 | L |
| ATOM | 1257 | CB | GLU | L | 165 | 25.090 | −3.132 | 4.544 | 1.00 | 14.25 | L |
| ATOM | 1258 | CG | GLU | L | 165 | 24.236 | −2.349 | 3.550 | 1.00 | 19.81 | L |
| ATOM | 1259 | CD | GLU | L | 165 | 23.094 | −3.160 | 2.940 | 1.00 | 26.59 | L |
| ATOM | 1260 | OE1 | GLU | L | 165 | 23.197 | −4.407 | 2.854 | 1.00 | 27.51 | L |
| ATOM | 1261 | OE2 | GLU | L | 165 | 22.091 | −2.540 | 2.524 | 1.00 | 29.07 | L |
| ATOM | 1262 | C | GLU | L | 165 | 25.385 | −4.453 | 6.631 | 1.00 | 13.72 | L |
| ATOM | 1263 | O | GLU | L | 165 | 25.865 | −3.898 | 7.631 | 1.00 | 12.63 | L |
| ATOM | 1264 | N | GLN | L | 166 | 25.735 | −5.675 | 6.256 | 1.00 | 14.08 | L |
| ATOM | 1265 | CA | GLN | L | 166 | 26.703 | −6.444 | 7.026 | 1.00 | 13.65 | L |
| ATOM | 1266 | CB | GLN | L | 166 | 26.928 | −7.808 | 6.367 | 1.00 | 13.01 | L |
| ATOM | 1267 | CG | GLN | L | 166 | 27.820 | −8.754 | 7.158 | 1.00 | 11.96 | L |
| ATOM | 1268 | CD | GLN | L | 166 | 27.728 | −10.194 | 6.661 | 1.00 | 13.28 | L |
| ATOM | 1269 | OE1 | GLN | L | 166 | 27.849 | −10.461 | 5.468 | 1.00 | 10.94 | L |
| ATOM | 1270 | NE2 | GLN | L | 166 | 27.513 | −11.125 | 7.583 | 1.00 | 11.12 | L |
| ATOM | 1271 | C | GLN | L | 166 | 28.019 | −5.668 | 7.134 | 1.00 | 14.98 | L |
| ATOM | 1272 | O | GLN | L | 166 | 28.527 | −5.146 | 6.141 | 1.00 | 13.40 | L |
| ATOM | 1273 | N | ASP | L | 167 | 28.556 | −5.577 | 8.347 | 1.00 | 16.22 | L |
| ATOM | 1274 | CA | ASP | L | 167 | 29.803 | −4.848 | 8.568 | 1.00 | 17.79 | L |
| ATOM | 1275 | CB | ASP | L | 167 | 30.128 | −4.791 | 10.065 | 1.00 | 19.17 | L |
| ATOM | 1276 | CG | ASP | L | 167 | 31.258 | −3.823 | 10.371 | 1.00 | 20.60 | L |
| ATOM | 1277 | OD1 | ASP | L | 167 | 31.005 | −2.600 | 10.410 | 1.00 | 22.68 | L |
| ATOM | 1278 | OD2 | ASP | L | 167 | 32.401 | −4.285 | 10.545 | 1.00 | 17.84 | L |
| ATOM | 1279 | C | ASP | L | 167 | 30.962 | −5.508 | 7.815 | 1.00 | 17.58 | L |
| ATOM | 1280 | O | ASP | L | 167 | 31.149 | −6.722 | 7.889 | 1.00 | 16.84 | L |
| ATOM | 1281 | N | SER | L | 168 | 31.748 | −4.705 | 7.104 | 1.00 | 18.23 | L |
| ATOM | 1282 | CA | SER | L | 168 | 32.866 | −5.228 | 6.325 | 1.00 | 21.22 | L |
| ATOM | 1283 | CB | SER | L | 168 | 33.408 | −4.154 | 5.378 | 1.00 | 24.29 | L |
| ATOM | 1284 | OG | SER | L | 168 | 34.019 | −3.099 | 6.101 | 1.00 | 29.19 | L |
| ATOM | 1285 | C | SER | L | 168 | 34.014 | −5.772 | 7.164 | 1.00 | 21.51 | L |
| ATOM | 1286 | O | SER | L | 168 | 34.825 | −6.552 | 6.671 | 1.00 | 21.73 | L |
| ATOM | 1287 | N | LYS | L | 169 | 34.078 | −5.373 | 8.430 | 1.00 | 21.65 | L |
| ATOM | 1288 | CA | LYS | L | 169 | 35.153 | −5.831 | 9.305 | 1.00 | 22.46 | L |
| ATOM | 1289 | CB | LYS | L | 169 | 35.662 | −4.671 | 10.168 | 1.00 | 26.57 | L |
| ATOM | 1290 | CG | LYS | L | 169 | 36.634 | −3.752 | 9.436 | 1.00 | 33.95 | L |
| ATOM | 1291 | CD | LYS | L | 169 | 36.932 | −2.480 | 10.227 | 1.00 | 38.71 | L |
| ATOM | 1292 | CE | LYS | L | 169 | 36.231 | −1.273 | 9.610 | 1.00 | 40.47 | L |
| ATOM | 1293 | NZ | LYS | L | 169 | 36.452 | −0.029 | 10.405 | 1.00 | 43.61 | L |
| ATOM | 1294 | C | LYS | L | 169 | 34.832 | −7.011 | 10.209 | 1.00 | 20.01 | L |
| ATOM | 1295 | O | LYS | L | 169 | 35.614 | −7.955 | 10.288 | 1.00 | 21.04 | L |
| ATOM | 1296 | N | ASP | L | 170 | 33.703 | −6.961 | 10.907 | 1.00 | 16.82 | L |
| ATOM | 1297 | CA | ASP | L | 170 | 33.363 | −8.054 | 11.808 | 1.00 | 15.97 | L |
| ATOM | 1298 | CB | ASP | L | 170 | 33.148 | −7.535 | 13.238 | 1.00 | 17.56 | L |
| ATOM | 1299 | CG | ASP | L | 170 | 31.976 | −6.567 | 13.355 | 1.00 | 19.32 | L |
| ATOM | 1300 | OD1 | ASP | L | 170 | 30.944 | −6.779 | 12.689 | 1.00 | 20.23 | L |
| ATOM | 1301 | OD2 | ASP | L | 170 | 32.087 | −5.600 | 14.136 | 1.00 | 25.27 | L |
| ATOM | 1302 | C | ASP | L | 170 | 32.159 | −8.874 | 11.359 | 1.00 | 14.02 | L |
| ATOM | 1303 | O | ASP | L | 170 | 31.698 | −9.751 | 12.085 | 1.00 | 13.80 | L |
| ATOM | 1304 | N | SER | L | 171 | 31.649 | −8.566 | 10.168 | 1.00 | 13.35 | L |
| ATOM | 1305 | CA | SER | L | 171 | 30.523 | −9.287 | 9.580 | 1.00 | 13.71 | L |
| ATOM | 1306 | CB | SER | L | 171 | 30.978 | −10.703 | 9.212 | 1.00 | 9.74 | L |
| ATOM | 1307 | OG | SER | L | 171 | 31.944 | −10.653 | 8.176 | 1.00 | 16.25 | L |
| ATOM | 1308 | C | SER | L | 171 | 29.229 | −9.362 | 10.396 | 1.00 | 12.01 | L |
| ATOM | 1309 | O | SER | L | 171 | 28.456 | −10.316 | 10.268 | 1.00 | 12.55 | L |
| ATOM | 1310 | N | THR | L | 172 | 28.983 | −8.367 | 11.230 | 1.00 | 10.84 | L |
| ATOM | 1311 | CA | THR | L | 172 | 27.765 | −8.369 | 12.015 | 1.00 | 12.48 | L |
| ATOM | 1312 | CB | THR | L | 172 | 28.027 | −7.954 | 13.477 | 1.00 | 14.43 | L |
| ATOM | 1313 | OG1 | THR | L | 172 | 28.551 | −6.617 | 13.516 | 1.00 | 14.43 | L |
| ATOM | 1314 | CG2 | THR | L | 172 | 29.009 | −8.922 | 14.130 | 1.00 | 14.03 | L |
| ATOM | 1315 | C | THR | L | 172 | 26.747 | −7.412 | 11.406 | 1.00 | 12.49 | L |
| ATOM | 1316 | O | THR | L | 172 | 27.014 | −6.750 | 10.401 | 1.00 | 11.97 | L |
| ATOM | 1317 | N | TYR | L | 173 | 25.575 | −7.366 | 12.024 | 1.00 | 13.14 | L |
| ATOM | 1318 | CA | TYR | L | 173 | 24.488 | −6.497 | 11.600 | 1.00 | 12.87 | L |
| ATOM | 1319 | CB | TYR | L | 173 | 23.279 | −7.329 | 11.130 | 1.00 | 12.50 | L |
| ATOM | 1320 | CG | TYR | L | 173 | 23.480 | −8.073 | 9.825 | 1.00 | 12.31 | L |
| ATOM | 1321 | CD1 | TYR | L | 173 | 23.252 | −7.447 | 8.596 | 1.00 | 12.78 | L |
| ATOM | 1322 | CE1 | TYR | L | 173 | 23.477 | −8.117 | 7.396 | 1.00 | 14.11 | L |
| ATOM | 1323 | CD2 | TYR | L | 173 | 23.935 | −9.395 | 9.817 | 1.00 | 14.56 | L |
| ATOM | 1324 | CE2 | TYR | L | 173 | 24.160 | −10.073 | 8.620 | 1.00 | 12.75 | L |
| ATOM | 1325 | CZ | TYR | L | 173 | 23.933 | −9.430 | 7.419 | 1.00 | 13.58 | L |
| ATOM | 1326 | OH | TYR | L | 173 | 24.185 | −10.090 | 6.242 | 1.00 | 16.09 | L |
| ATOM | 1327 | C | TYR | L | 173 | 24.086 | −5.706 | 12.838 | 1.00 | 12.88 | L |
| ATOM | 1328 | O | TYR | L | 173 | 24.343 | −6.128 | 13.959 | 1.00 | 11.85 | L |
| ATOM | 1329 | N | SER | L | 174 | 23.464 | −4.555 | 12.631 | 1.00 | 11.94 | L |
| ATOM | 1330 | CA | SER | L | 174 | 22.985 | −3.753 | 13.738 | 1.00 | 12.17 | L |
| ATOM | 1331 | CB | SER | L | 174 | 23.907 | −2.560 | 13.993 | 1.00 | 13.78 | L |
| ATOM | 1332 | OG | SER | L | 174 | 25.096 | −3.006 | 14.625 | 1.00 | 13.68 | L |
| ATOM | 1333 | C | SER | L | 174 | 21.585 | −3.295 | 13.382 | 1.00 | 11.50 | L |
| ATOM | 1334 | O | SER | L | 174 | 21.271 | −3.054 | 12.215 | 1.00 | 10.58 | L |
| ATOM | 1335 | N | LEU | L | 175 | 20.733 | −3.199 | 14.388 | 1.00 | 10.45 | L |
| ATOM | 1336 | CA | LEU | L | 175 | 19.364 | −2.798 | 14.142 | 1.00 | 12.24 | L |
| ATOM | 1337 | CB | LEU | L | 175 | 18.442 | −4.014 | 14.312 | 1.00 | 10.60 | L |
| ATOM | 1338 | CG | LEU | L | 175 | 16.930 | −3.851 | 14.130 | 1.00 | 13.37 | L |
| ATOM | 1339 | CD1 | LEU | L | 175 | 16.292 | −5.230 | 13.930 | 1.00 | 13.60 | L |
| ATOM | 1340 | CD2 | LEU | L | 175 | 16.324 | −3.137 | 15.332 | 1.00 | 13.31 | L |
| ATOM | 1341 | C | LEU | L | 175 | 18.949 | −1.666 | 15.063 | 1.00 | 11.61 | L |
| ATOM | 1342 | O | LEU | L | 175 | 19.322 | −1.630 | 16.233 | 1.00 | 12.78 | L |
| ATOM | 1343 | N | SER | L | 176 | 18.182 | −0.737 | 14.514 | 1.00 | 11.83 | L |
| ATOM | 1344 | CA | SER | L | 176 | 17.695 | 0.396 | 15.275 | 1.00 | 10.87 | L |
| ATOM | 1345 | CB | SER | L | 176 | 18.339 | 1.693 | 14.770 | 1.00 | 10.41 | L |
| ATOM | 1346 | OG | SER | L | 176 | 17.687 | 2.813 | 15.331 | 1.00 | 10.74 | L |
| ATOM | 1347 | C | SER | L | 176 | 16.179 | 0.486 | 15.138 | 1.00 | 12.51 | L |
| ATOM | 1348 | O | SER | L | 176 | 15.635 | 0.431 | 14.033 | 1.00 | 10.10 | L |
| ATOM | 1349 | N | SER | L | 177 | 15.500 | 0.626 | 16.266 | 1.00 | 11.57 | L |
| ATOM | 1350 | CA | SER | L | 177 | 14.051 | 0.726 | 16.255 | 1.00 | 12.29 | L |
| ATOM | 1351 | CB | SER | L | 177 | 13.423 | −0.529 | 16.859 | 1.00 | 9.97 | L |
| ATOM | 1352 | OG | SER | L | 177 | 12.019 | −0.383 | 16.950 | 1.00 | 16.03 | L |
| ATOM | 1353 | C | SER | L | 177 | 13.610 | 1.943 | 17.050 | 1.00 | 13.15 | L |
| ATOM | 1354 | O | SER | L | 177 | 14.128 | 2.210 | 18.130 | 1.00 | 11.32 | L |
| ATOM | 1355 | N | THR | L | 178 | 12.646 | 2.674 | 16.506 | 1.00 | 11.77 | L |
| ATOM | 1356 | CA | THR | L | 178 | 12.143 | 3.862 | 17.168 | 1.00 | 13.82 | L |
| ATOM | 1357 | CB | THR | L | 178 | 12.302 | 5.107 | 16.285 | 1.00 | 13.96 | L |
| ATOM | 1358 | OG1 | THR | L | 178 | 13.675 | 5.268 | 15.927 | 1.00 | 16.45 | L |
| ATOM | 1359 | CG2 | THR | L | 178 | 11.821 | 6.349 | 17.028 | 1.00 | 12.82 | L |
| ATOM | 1360 | C | THR | L | 178 | 10.671 | 3.734 | 17.504 | 1.00 | 14.51 | L |
| ATOM | 1361 | O | THR | L | 178 | 9.863 | 3.358 | 16.655 | 1.00 | 16.37 | L |
| ATOM | 1362 | N | LEU | L | 179 | 10.339 | 4.051 | 18.750 | 1.00 | 16.63 | L |
| ATOM | 1363 | CA | LEU | L | 179 | 8.963 | 4.034 | 19.233 | 1.00 | 16.69 | L |
| ATOM | 1364 | CB | LEU | L | 179 | 8.884 | 3.359 | 20.604 | 1.00 | 16.86 | L |
| ATOM | 1365 | CG | LEU | L | 179 | 7.534 | 3.458 | 21.315 | 1.00 | 19.43 | L |
| ATOM | 1366 | CD1 | LEU | L | 179 | 6.543 | 2.511 | 20.666 | 1.00 | 21.29 | L |
| ATOM | 1367 | CD2 | LEU | L | 179 | 7.702 | 3.118 | 22.783 | 1.00 | 19.97 | L |
| ATOM | 1368 | C | LEU | L | 179 | 8.604 | 5.510 | 19.356 | 1.00 | 16.80 | L |
| ATOM | 1369 | O | LEU | L | 179 | 9.350 | 6.281 | 19.960 | 1.00 | 16.82 | L |
| ATOM | 1370 | N | THR | L | 180 | 7.475 | 5.911 | 18.788 | 1.00 | 17.93 | L |
| ATOM | 1371 | CA | THR | L | 180 | 7.085 | 7.315 | 18.823 | 1.00 | 19.06 | L |
| ATOM | 1372 | CB | THR | L | 180 | 7.132 | 7.927 | 17.403 | 1.00 | 21.34 | L |
| ATOM | 1373 | OG1 | THR | L | 180 | 8.404 | 7.647 | 16.803 | 1.00 | 24.31 | L |
| ATOM | 1374 | CG2 | THR | L | 180 | 6.950 | 9.436 | 17.467 | 1.00 | 22.45 | L |
| ATOM | 1375 | C | THR | L | 180 | 5.694 | 7.538 | 19.405 | 1.00 | 19.23 | L |
| ATOM | 1376 | O | THR | L | 180 | 4.743 | 6.861 | 19.039 | 1.00 | 19.97 | L |
| ATOM | 1377 | N | LEU | L | 181 | 5.594 | 8.482 | 20.331 | 1.00 | 20.20 | L |
| ATOM | 1378 | CA | LEU | L | 181 | 4.321 | 8.809 | 20.967 | 1.00 | 21.49 | L |
| ATOM | 1379 | CB | LEU | L | 181 | 4.206 | 8.138 | 22.346 | 1.00 | 21.07 | L |
| ATOM | 1380 | CG | LEU | L | 181 | 4.106 | 6.615 | 22.492 | 1.00 | 25.01 | L |
| ATOM | 1381 | CD1 | LEU | L | 181 | 5.335 | 5.941 | 21.922 | 1.00 | 26.92 | L |
| ATOM | 1382 | CD2 | LEU | L | 181 | 3.971 | 6.265 | 23.967 | 1.00 | 22.84 | L |
| ATOM | 1383 | C | LEU | L | 181 | 4.256 | 10.318 | 21.157 | 1.00 | 21.74 | L |
| ATOM | 1384 | O | LEU | L | 181 | 5.281 | 10.990 | 21.156 | 1.00 | 22.89 | L |
| ATOM | 1385 | N | SER | L | 182 | 3.053 | 10.854 | 21.314 | 1.00 | 22.26 | L |
| ATOM | 1386 | CA | SER | L | 182 | 2.923 | 12.282 | 21.558 | 1.00 | 21.84 | L |
| ATOM | 1387 | CB | SER | L | 182 | 1.465 | 12.709 | 21.479 | 1.00 | 20.84 | L |
| ATOM | 1388 | OG | SER | L | 182 | 0.722 | 12.081 | 22.506 | 1.00 | 22.43 | L |
| ATOM | 1389 | C | SER | L | 182 | 3.419 | 12.465 | 22.988 | 1.00 | 22.36 | L |
| ATOM | 1390 | O | SER | L | 182 | 3.435 | 11.510 | 23.773 | 1.00 | 20.23 | L |
| ATOM | 1391 | N | LYS | L | 183 | 3.827 | 13.678 | 23.336 | 1.00 | 23.03 | L |
| ATOM | 1392 | CA | LYS | L | 183 | 4.299 | 13.923 | 24.688 | 1.00 | 24.43 | L |
| ATOM | 1393 | CB | LYS | L | 183 | 4.619 | 15.407 | 24.866 | 1.00 | 26.77 | L |
| ATOM | 1394 | CG | LYS | L | 183 | 5.138 | 15.758 | 26.243 | 1.00 | 28.30 | L |
| ATOM | 1395 | CD | LYS | L | 183 | 5.438 | 17.237 | 26.352 | 1.00 | 30.18 | L |
| ATOM | 1396 | CE | LYS | L | 183 | 5.870 | 17.603 | 27.762 | 1.00 | 34.74 | L |
| ATOM | 1397 | NZ | LYS | L | 183 | 6.127 | 19.064 | 27.889 | 1.00 | 36.80 | L |
| ATOM | 1398 | C | LYS | L | 183 | 3.231 | 13.481 | 25.700 | 1.00 | 25.12 | L |
| ATOM | 1399 | O | LYS | L | 183 | 3.510 | 12.711 | 26.623 | 1.00 | 25.79 | L |
| ATOM | 1400 | N | ALA | L | 184 | 2.004 | 13.955 | 25.504 | 1.00 | 23.69 | L |
| ATOM | 1401 | CA | ALA | L | 184 | 0.894 | 13.628 | 26.394 | 1.00 | 23.21 | L |
| ATOM | 1402 | CB | ALA | L | 184 | −0.413 | 14.203 | 25.838 | 1.00 | 21.70 | L |
| ATOM | 1403 | C | ALA | L | 184 | 0.741 | 12.133 | 26.634 | 1.00 | 22.70 | L |
| ATOM | 1404 | O | ALA | L | 184 | 0.594 | 11.703 | 27.773 | 1.00 | 24.31 | L |
| ATOM | 1405 | N | ASP | L | 185 | 0.765 | 11.336 | 25.569 | 1.00 | 22.02 | L |
| ATOM | 1406 | CA | ASP | L | 185 | 0.620 | 9.892 | 25.739 | 1.00 | 21.79 | L |
| ATOM | 1407 | CB | ASP | L | 185 | 0.433 | 9.209 | 24.380 | 1.00 | 22.72 | L |
| ATOM | 1408 | CG | ASP | L | 185 | −0.968 | 9.400 | 23.822 | 1.00 | 25.29 | L |
| ATOM | 1409 | OD1 | ASP | L | 185 | −1.173 | 9.177 | 22.610 | 1.00 | 26.57 | L |
| ATOM | 1410 | OD2 | ASP | L | 185 | −1.869 | 9.772 | 24.606 | 1.00 | 26.31 | L |
| ATOM | 1411 | C | ASP | L | 185 | 1.832 | 9.314 | 26.455 | 1.00 | 21.82 | L |
| ATOM | 1412 | O | ASP | L | 185 | 1.717 | 8.378 | 27.252 | 1.00 | 20.30 | L |
| ATOM | 1413 | N | TYR | L | 186 | 2.992 | 9.897 | 26.180 | 1.00 | 21.62 | L |
| ATOM | 1414 | CA | TYR | L | 186 | 4.236 | 9.450 | 26.777 | 1.00 | 22.76 | L |
| ATOM | 1415 | CB | TYR | L | 186 | 5.391 | 10.280 | 26.220 | 1.00 | 22.18 | L |
| ATOM | 1416 | CG | TYR | L | 186 | 6.722 | 9.990 | 26.861 | 1.00 | 20.97 | L |
| ATOM | 1417 | CD1 | TYR | L | 186 | 7.332 | 8.745 | 26.715 | 1.00 | 21.21 | L |
| ATOM | 1418 | CE1 | TYR | L | 186 | 8.551 | 8.466 | 27.321 | 1.00 | 19.08 | L |
| ATOM | 1419 | CD2 | TYR | L | 186 | 7.367 | 10.958 | 27.633 | 1.00 | 19.68 | L |
| ATOM | 1420 | CE2 | TYR | L | 186 | 8.590 | 10.686 | 28.248 | 1.00 | 18.83 | L |
| ATOM | 1421 | CZ | TYR | L | 186 | 9.173 | 9.437 | 28.086 | 1.00 | 18.09 | L |
| ATOM | 1422 | OH | TYR | L | 186 | 10.371 | 9.159 | 28.702 | 1.00 | 20.18 | L |
| ATOM | 1423 | C | TYR | L | 186 | 4.191 | 9.566 | 28.298 | 1.00 | 24.18 | L |
| ATOM | 1424 | O | TYR | L | 186 | 4.688 | 8.696 | 29.012 | 1.00 | 23.51 | L |
| ATOM | 1425 | N | GLU | L | 187 | 3.558 | 10.629 | 28.783 | 1.00 | 24.83 | L |
| ATOM | 1426 | CA | GLU | L | 187 | 3.465 | 10.889 | 30.213 | 1.00 | 26.90 | L |
| ATOM | 1427 | CB | GLU | L | 187 | 3.301 | 12.388 | 30.443 | 1.00 | 28.96 | L |
| ATOM | 1428 | CG | GLU | L | 187 | 4.394 | 13.189 | 29.791 | 1.00 | 34.27 | L |
| ATOM | 1429 | CD | GLU | L | 187 | 4.223 | 14.665 | 30.008 | 1.00 | 38.80 | L |
| ATOM | 1430 | OE1 | GLU | L | 187 | 3.123 | 15.183 | 29.713 | 1.00 | 42.16 | L |
| ATOM | 1431 | OE2 | GLU | L | 187 | 5.190 | 15.306 | 30.470 | 1.00 | 40.51 | L |
| ATOM | 1432 | C | GLU | L | 187 | 2.364 | 10.140 | 30.944 | 1.00 | 25.34 | L |
| ATOM | 1433 | O | GLU | L | 187 | 2.193 | 10.305 | 32.146 | 1.00 | 23.76 | L |
| ATOM | 1434 | N | LYS | L | 188 | 1.615 | 9.320 | 30.221 | 1.00 | 25.58 | L |
| ATOM | 1435 | CA | LYS | L | 188 | 0.545 | 8.549 | 30.835 | 1.00 | 24.49 | L |
| ATOM | 1436 | CB | LYS | L | 188 | −0.611 | 8.398 | 29.845 | 1.00 | 26.09 | L |
| ATOM | 1437 | CG | LYS | L | 188 | −1.285 | 9.726 | 29.525 | 1.00 | 28.85 | L |
| ATOM | 1438 | CD | LYS | L | 188 | −2.012 | 9.706 | 28.192 | 1.00 | 32.43 | L |
| ATOM | 1439 | CE | LYS | L | 188 | −3.142 | 8.701 | 28.180 | 1.00 | 34.73 | L |
| ATOM | 1440 | NZ | LYS | L | 188 | −3.840 | 8.688 | 26.863 | 1.00 | 38.21 | L |
| ATOM | 1441 | C | LYS | L | 188 | 1.061 | 7.182 | 31.258 | 1.00 | 22.63 | L |
| ATOM | 1442 | O | LYS | L | 188 | 0.380 | 6.452 | 31.971 | 1.00 | 25.15 | L |
| ATOM | 1443 | N | HIS | L | 189 | 2.279 | 6.853 | 30.839 | 1.00 | 19.65 | L |
| ATOM | 1444 | CA | HIS | L | 189 | 2.877 | 5.554 | 31.141 | 1.00 | 17.50 | L |
| ATOM | 1445 | CB | HIS | L | 189 | 3.080 | 4.793 | 29.834 | 1.00 | 18.48 | L |
| ATOM | 1446 | CG | HIS | L | 189 | 1.862 | 4.781 | 28.966 | 1.00 | 19.62 | L |
| ATOM | 1447 | CD2 | HIS | L | 189 | 1.609 | 5.375 | 27.776 | 1.00 | 19.10 | L |
| ATOM | 1448 | ND1 | HIS | L | 189 | 0.698 | 4.142 | 29.330 | 1.00 | 20.81 | L |
| ATOM | 1449 | CE1 | HIS | L | 189 | −0.221 | 4.342 | 28.402 | 1.00 | 19.90 | L |
| ATOM | 1450 | NE2 | HIS | L | 189 | 0.307 | 5.087 | 27.448 | 1.00 | 20.89 | L |
| ATOM | 1451 | C | HIS | L | 189 | 4.189 | 5.657 | 31.905 | 1.00 | 17.20 | L |
| ATOM | 1452 | O | HIS | L | 189 | 4.858 | 6.693 | 31.878 | 1.00 | 18.52 | L |
| ATOM | 1453 | N | LYS | L | 190 | 4.569 | 4.564 | 32.564 | 1.00 | 18.14 | L |
| ATOM | 1454 | CA | LYS | L | 190 | 5.772 | 4.559 | 33.380 | 1.00 | 17.10 | L |
| ATOM | 1455 | CB | LYS | L | 190 | 5.410 | 4.116 | 34.801 | 1.00 | 18.82 | L |
| ATOM | 1456 | CG | LYS | L | 190 | 6.579 | 4.139 | 35.776 | 1.00 | 20.80 | L |
| ATOM | 1457 | CD | LYS | L | 190 | 7.211 | 5.528 | 35.879 | 1.00 | 19.19 | L |
| ATOM | 1458 | CE | LYS | L | 190 | 6.238 | 6.546 | 36.459 | 1.00 | 19.25 | L |
| ATOM | 1459 | NZ | LYS | L | 190 | 6.855 | 7.892 | 36.623 | 1.00 | 17.30 | L |
| ATOM | 1460 | C | LYS | L | 190 | 6.970 | 3.749 | 32.886 | 1.00 | 17.17 | L |
| ATOM | 1461 | O | LYS | L | 190 | 8.059 | 4.296 | 32.725 | 1.00 | 15.91 | L |
| ATOM | 1462 | N | VAL | L | 191 | 6.780 | 2.452 | 32.661 | 1.00 | 16.37 | L |
| ATOM | 1463 | CA | VAL | L | 191 | 7.871 | 1.598 | 32.217 | 1.00 | 16.10 | L |
| ATOM | 1464 | CB | VAL | L | 191 | 7.765 | 0.182 | 32.843 | 1.00 | 15.81 | L |
| ATOM | 1465 | CG1 | VAL | L | 191 | 9.005 | −0.639 | 32.505 | 1.00 | 11.50 | L |
| ATOM | 1466 | CG2 | VAL | L | 191 | 7.602 | 0.287 | 34.355 | 1.00 | 15.84 | L |
| ATOM | 1467 | C | VAL | L | 191 | 7.939 | 1.457 | 30.696 | 1.00 | 19.05 | L |
| ATOM | 1468 | O | VAL | L | 191 | 6.972 | 1.038 | 30.058 | 1.00 | 21.01 | L |
| ATOM | 1469 | N | TYR | L | 192 | 9.081 | 1.820 | 30.119 | 1.00 | 17.53 | L |
| ATOM | 1470 | CA | TYR | L | 192 | 9.283 | 1.710 | 28.678 | 1.00 | 18.92 | L |
| ATOM | 1471 | CB | TYR | L | 192 | 9.725 | 3.056 | 28.088 | 1.00 | 16.63 | L |
| ATOM | 1472 | CG | TYR | L | 192 | 8.582 | 4.034 | 27.978 | 1.00 | 17.28 | L |
| ATOM | 1473 | CD1 | TYR | L | 192 | 8.101 | 4.711 | 29.100 | 1.00 | 17.49 | L |
| ATOM | 1474 | CE1 | TYR | L | 192 | 6.986 | 5.554 | 29.006 | 1.00 | 19.06 | L |
| ATOM | 1475 | CD2 | TYR | L | 192 | 7.924 | 4.226 | 26.760 | 1.00 | 17.81 | L |
| ATOM | 1476 | CE2 | TYR | L | 192 | 6.821 | 5.057 | 26.656 | 1.00 | 17.43 | L |
| ATOM | 1477 | CZ | TYR | L | 192 | 6.354 | 5.719 | 27.775 | 1.00 | 20.35 | L |
| ATOM | 1478 | OH | TYR | L | 192 | 5.261 | 6.550 | 27.652 | 1.00 | 18.73 | L |
| ATOM | 1479 | C | TYR | L | 192 | 10.337 | 0.631 | 28.443 | 1.00 | 19.66 | L |
| ATOM | 1480 | O | TYR | L | 192 | 11.474 | 0.746 | 28.898 | 1.00 | 21.16 | L |
| ATOM | 1481 | N | ALA | L | 193 | 9.954 | −0.424 | 27.738 | 1.00 | 18.53 | L |
| ATOM | 1482 | CA | ALA | L | 193 | 10.869 | −1.529 | 27.515 | 1.00 | 19.36 | L |
| ATOM | 1483 | CB | ALA | L | 193 | 10.486 | −2.697 | 28.421 | 1.00 | 17.12 | L |
| ATOM | 1484 | C | ALA | L | 193 | 10.979 | −2.014 | 26.081 | 1.00 | 18.09 | L |
| ATOM | 1485 | O | ALA | L | 193 | 10.028 | −1.975 | 25.314 | 1.00 | 17.69 | L |
| ATOM | 1486 | N | CYS | L | 194 | 12.172 | −2.492 | 25.758 | 1.00 | 20.09 | L |
| ATOM | 1487 | CA | CYS | L | 194 | 12.503 | −3.026 | 24.454 | 1.00 | 20.71 | L |
| ATOM | 1488 | C | CYS | L | 194 | 12.911 | −4.472 | 24.703 | 1.00 | 18.66 | L |
| ATOM | 1489 | O | CYS | L | 194 | 13.911 | −4.718 | 25.363 | 1.00 | 17.74 | L |
| ATOM | 1490 | CB | CYS | L | 194 | 13.685 | −2.248 | 23.868 | 1.00 | 22.56 | L |
| ATOM | 1491 | SG | CYS | L | 194 | 14.215 | −2.874 | 22.252 | 1.00 | 33.28 | L |
| ATOM | 1492 | N | GLU | L | 195 | 12.142 | −5.423 | 24.177 | 1.00 | 21.04 | L |
| ATOM | 1493 | CA | GLU | L | 195 | 12.429 | −6.845 | 24.367 | 1.00 | 19.41 | L |
| ATOM | 1494 | CB | GLU | L | 195 | 11.141 | −7.574 | 24.750 | 1.00 | 22.30 | L |
| ATOM | 1495 | CG | GLU | L | 195 | 11.331 | −9.005 | 25.203 | 1.00 | 26.50 | L |
| ATOM | 1496 | CD | GLU | L | 195 | 10.029 | −9.622 | 25.680 | 1.00 | 29.99 | L |
| ATOM | 1497 | OE1 | GLU | L | 195 | 9.351 | −9.009 | 26.529 | 1.00 | 32.22 | L |
| ATOM | 1498 | OE2 | GLU | L | 195 | 9.682 | −10.717 | 25.207 | 1.00 | 35.42 | L |
| ATOM | 1499 | C | GLU | L | 195 | 13.027 | −7.453 | 23.102 | 1.00 | 18.48 | L |
| ATOM | 1500 | O | GLU | L | 195 | 12.443 | −7.377 | 22.025 | 1.00 | 17.02 | L |
| ATOM | 1501 | N | VAL | L | 196 | 14.182 | −8.089 | 23.245 | 1.00 | 18.35 | L |
| ATOM | 1502 | CA | VAL | L | 196 | 14.876 | −8.653 | 22.098 | 1.00 | 17.86 | L |
| ATOM | 1503 | CB | VAL | L | 196 | 16.288 | −8.056 | 21.988 | 1.00 | 15.95 | L |
| ATOM | 1504 | CG1 | VAL | L | 196 | 17.028 | −8.671 | 20.806 | 1.00 | 16.67 | L |
| ATOM | 1505 | CG2 | VAL | L | 196 | 16.204 | −6.539 | 21.868 | 1.00 | 15.01 | L |
| ATOM | 1506 | C | VAL | L | 196 | 15.016 | −10.167 | 22.061 | 1.00 | 19.05 | L |
| ATOM | 1507 | O | VAL | L | 196 | 15.306 | −10.806 | 23.070 | 1.00 | 18.59 | L |
| ATOM | 1508 | N | THR | L | 197 | 14.818 | −10.723 | 20.870 | 1.00 | 20.36 | L |
| ATOM | 1509 | CA | THR | L | 197 | 14.950 | −12.155 | 20.638 | 1.00 | 23.34 | L |
| ATOM | 1510 | CB | THR | L | 197 | 13.600 | −12.783 | 20.241 | 1.00 | 25.08 | L |
| ATOM | 1511 | OG1 | THR | L | 197 | 12.721 | −12.760 | 21.373 | 1.00 | 27.06 | L |
| ATOM | 1512 | CG2 | THR | L | 197 | 13.794 | −14.217 | 19.776 | 1.00 | 28.18 | L |
| ATOM | 1513 | C | THR | L | 197 | 15.953 | −12.347 | 19.504 | 1.00 | 22.30 | L |
| ATOM | 1514 | O | THR | L | 197 | 15.930 | −11.612 | 18.517 | 1.00 | 24.01 | L |
| ATOM | 1515 | N | HIS | L | 198 | 16.829 | −13.335 | 19.648 | 1.00 | 20.96 | L |
| ATOM | 1516 | CA | HIS | L | 198 | 17.860 | −13.610 | 18.654 | 1.00 | 23.55 | L |
| ATOM | 1517 | CB | HIS | L | 198 | 19.009 | −12.605 | 18.818 | 1.00 | 20.62 | L |
| ATOM | 1518 | CG | HIS | L | 198 | 20.106 | −12.758 | 17.811 | 1.00 | 18.51 | L |
| ATOM | 1519 | CD2 | HIS | L | 198 | 21.331 | −13.328 | 17.907 | 1.00 | 15.47 | L |
| ATOM | 1520 | ND1 | HIS | L | 198 | 20.005 | −12.281 | 16.522 | 1.00 | 19.76 | L |
| ATOM | 1521 | CE1 | HIS | L | 198 | 21.121 | −12.549 | 15.867 | 1.00 | 15.78 | L |
| ATOM | 1522 | NE2 | HIS | L | 198 | 21.941 | −13.185 | 16.685 | 1.00 | 19.44 | L |
| ATOM | 1523 | C | HIS | L | 198 | 18.391 | −15.026 | 18.867 | 1.00 | 24.96 | L |
| ATOM | 1524 | O | HIS | L | 198 | 18.426 | −15.512 | 19.994 | 1.00 | 26.99 | L |
| ATOM | 1525 | N | GLN | L | 199 | 18.805 | −15.676 | 17.784 | 1.00 | 25.79 | L |
| ATOM | 1526 | CA | GLN | L | 199 | 19.341 | −17.036 | 17.844 | 1.00 | 28.35 | L |
| ATOM | 1527 | CB | GLN | L | 199 | 19.898 | −17.432 | 16.469 | 1.00 | 29.94 | L |
| ATOM | 1528 | CG | GLN | L | 199 | 21.016 | −18.481 | 16.474 | 1.00 | 34.66 | L |
| ATOM | 1529 | CD | GLN | L | 199 | 20.511 | −19.906 | 16.596 | 1.00 | 38.44 | L |
| ATOM | 1530 | OE1 | GLN | L | 199 | 19.305 | −20.152 | 16.627 | 1.00 | 40.82 | L |
| ATOM | 1531 | NE2 | GLN | L | 199 | 21.438 | −20.858 | 16.658 | 1.00 | 37.44 | L |
| ATOM | 1532 | C | GLN | L | 199 | 20.430 | −17.166 | 18.901 | 1.00 | 27.35 | L |
| ATOM | 1533 | O | GLN | L | 199 | 20.606 | −18.230 | 19.493 | 1.00 | 28.08 | L |
| ATOM | 1534 | N | GLY | L | 200 | 21.153 | −16.075 | 19.139 | 1.00 | 27.05 | L |
| ATOM | 1535 | CA | GLY | L | 200 | 22.229 | −16.092 | 20.117 | 1.00 | 25.67 | L |
| ATOM | 1536 | C | GLY | L | 200 | 21.823 | −15.841 | 21.558 | 1.00 | 25.48 | L |
| ATOM | 1537 | O | GLY | L | 200 | 22.678 | −15.714 | 22.434 | 1.00 | 24.36 | L |
| ATOM | 1538 | N | LEU | L | 201 | 20.523 | −15.761 | 21.813 | 1.00 | 26.08 | L |
| ATOM | 1539 | CA | LEU | L | 201 | 20.029 | −15.536 | 23.168 | 1.00 | 27.93 | L |
| ATOM | 1540 | CB | LEU | L | 201 | 19.253 | −14.212 | 23.239 | 1.00 | 25.99 | L |
| ATOM | 1541 | CG | LEU | L | 201 | 20.037 | −12.939 | 22.890 | 1.00 | 27.23 | L |
| ATOM | 1542 | CD1 | LEU | L | 201 | 19.084 | −11.753 | 22.798 | 1.00 | 23.64 | L |
| ATOM | 1543 | CD2 | LEU | L | 201 | 21.112 | −12.690 | 23.947 | 1.00 | 25.84 | L |
| ATOM | 1544 | C | LEU | L | 201 | 19.115 | −16.696 | 23.549 | 1.00 | 28.82 | L |
| ATOM | 1545 | O | LEU | L | 201 | 18.145 | −16.977 | 22.847 | 1.00 | 29.55 | L |
| ATOM | 1546 | N | SER | L | 202 | 19.423 | −17.367 | 24.657 | 1.00 | 31.68 | L |
| ATOM | 1547 | CA | SER | L | 202 | 18.618 | −18.502 | 25.107 | 1.00 | 33.26 | L |
| ATOM | 1548 | CB | SER | L | 202 | 19.364 | −19.292 | 26.190 | 1.00 | 34.54 | L |
| ATOM | 1549 | OG | SER | L | 202 | 19.992 | −18.433 | 27.123 | 1.00 | 36.22 | L |
| ATOM | 1550 | C | SER | L | 202 | 17.246 | −18.070 | 25.609 | 1.00 | 33.32 | L |
| ATOM | 1551 | O | SER | L | 202 | 16.325 | −18.880 | 25.723 | 1.00 | 36.59 | L |
| ATOM | 1552 | N | SER | L | 203 | 17.115 | −16.786 | 25.908 | 1.00 | 32.11 | L |
| ATOM | 1553 | CA | SER | L | 203 | 15.852 | −16.230 | 26.368 | 1.00 | 30.35 | L |
| ATOM | 1554 | CB | SER | L | 203 | 15.656 | −16.476 | 27.868 | 1.00 | 31.50 | L |
| ATOM | 1555 | OG | SER | L | 203 | 16.715 | −15.916 | 28.621 | 1.00 | 32.19 | L |
| ATOM | 1556 | C | SER | L | 203 | 15.882 | −14.738 | 26.070 | 1.00 | 27.60 | L |
| ATOM | 1557 | O | SER | L | 203 | 16.950 | −14.140 | 25.977 | 1.00 | 26.30 | L |
| ATOM | 1558 | N | PRO | L | 204 | 14.702 | −14.120 | 25.916 | 1.00 | 26.58 | L |
| ATOM | 1559 | CD | PRO | L | 204 | 13.380 | −14.743 | 26.094 | 1.00 | 26.56 | L |
| ATOM | 1560 | CA | PRO | L | 204 | 14.564 | −12.691 | 25.619 | 1.00 | 25.72 | L |
| ATOM | 1561 | CB | PRO | L | 204 | 13.067 | −12.446 | 25.787 | 1.00 | 27.36 | L |
| ATOM | 1562 | CG | PRO | L | 204 | 12.469 | −13.757 | 25.398 | 1.00 | 26.88 | L |
| ATOM | 1563 | C | PRO | L | 204 | 15.390 | −11.770 | 26.514 | 1.00 | 25.08 | L |
| ATOM | 1564 | O | PRO | L | 204 | 15.509 | −11.995 | 27.721 | 1.00 | 24.51 | L |
| ATOM | 1565 | N | VAL | L | 205 | 15.970 | −10.740 | 25.907 | 1.00 | 23.58 | L |
| ATOM | 1566 | CA | VAL | L | 205 | 16.749 | −9.757 | 26.643 | 1.00 | 22.26 | L |
| ATOM | 1567 | CB | VAL | L | 205 | 18.104 | −9.459 | 25.948 | 1.00 | 22.35 | L |
| ATOM | 1568 | CG1 | VAL | L | 205 | 18.729 | −8.195 | 26.525 | 1.00 | 21.38 | L |
| ATOM | 1569 | CG2 | VAL | L | 205 | 19.054 | −10.635 | 26.145 | 1.00 | 22.65 | L |
| ATOM | 1570 | C | VAL | L | 205 | 15.903 | −8.487 | 26.688 | 1.00 | 21.61 | L |
| ATOM | 1571 | O | VAL | L | 205 | 15.462 | −7.986 | 25.657 | 1.00 | 19.60 | L |
| ATOM | 1572 | N | THR | L | 206 | 15.667 | −7.974 | 27.887 | 1.00 | 21.96 | L |
| ATOM | 1573 | CA | THR | L | 206 | 14.868 | −6.772 | 28.031 | 1.00 | 21.86 | L |
| ATOM | 1574 | CB | THR | L | 206 | 13.666 | −7.012 | 28.957 | 1.00 | 21.93 | L |
| ATOM | 1575 | OG1 | THR | L | 206 | 12.845 | −8.049 | 28.409 | 1.00 | 24.43 | L |
| ATOM | 1576 | CG2 | THR | L | 206 | 12.845 | −5.738 | 29.101 | 1.00 | 21.61 | L |
| ATOM | 1577 | C | THR | L | 206 | 15.657 | −5.597 | 28.582 | 1.00 | 20.36 | L |
| ATOM | 1578 | O | THR | L | 206 | 16.386 | −5.730 | 29.558 | 1.00 | 21.04 | L |
| ATOM | 1579 | N | LYS | L | 207 | 15.500 | −4.446 | 27.944 | 1.00 | 18.93 | L |
| ATOM | 1580 | CA | LYS | L | 207 | 16.161 | −3.228 | 28.383 | 1.00 | 19.68 | L |
| ATOM | 1581 | CB | LYS | L | 207 | 17.146 | −2.728 | 27.327 | 1.00 | 20.97 | L |
| ATOM | 1582 | CG | LYS | L | 207 | 18.583 | −2.701 | 27.802 | 1.00 | 25.24 | L |
| ATOM | 1583 | CD | LYS | L | 207 | 19.049 | −4.081 | 28.212 | 1.00 | 27.63 | L |
| ATOM | 1584 | CE | LYS | L | 207 | 20.510 | −4.072 | 28.606 | 1.00 | 30.08 | L |
| ATOM | 1585 | NZ | LYS | L | 207 | 20.985 | −5.429 | 28.957 | 1.00 | 28.65 | L |
| ATOM | 1586 | C | LYS | L | 207 | 15.053 | −2.212 | 28.574 | 1.00 | 19.24 | L |
| ATOM | 1587 | O | LYS | L | 207 | 14.189 | −2.049 | 27.706 | 1.00 | 18.03 | L |
| ATOM | 1588 | N | SER | L | 208 | 15.072 | −1.530 | 29.710 | 1.00 | 19.41 | L |
| ATOM | 1589 | CA | SER | L | 208 | 14.039 | −0.558 | 29.992 | 1.00 | 20.00 | L |
| ATOM | 1590 | CB | SER | L | 208 | 12.882 | −1.253 | 30.694 | 1.00 | 22.50 | L |
| ATOM | 1591 | OG | SER | L | 208 | 13.310 | −1.762 | 31.940 | 1.00 | 25.37 | L |
| ATOM | 1592 | C | SER | L | 208 | 14.499 | 0.599 | 30.854 | 1.00 | 19.05 | L |
| ATOM | 1593 | O | SER | L | 208 | 15.613 | 0.610 | 31.372 | 1.00 | 19.18 | L |
| ATOM | 1594 | N | PHE | L | 209 | 13.616 | 1.579 | 30.988 | 1.00 | 16.95 | L |
| ATOM | 1595 | CA | PHE | L | 209 | 13.857 | 2.736 | 31.831 | 1.00 | 18.01 | L |
| ATOM | 1596 | CB | PHE | L | 209 | 14.579 | 3.861 | 31.060 | 1.00 | 15.74 | L |
| ATOM | 1597 | CG | PHE | L | 209 | 13.722 | 4.571 | 30.048 | 1.00 | 14.93 | L |
| ATOM | 1598 | CD1 | PHE | L | 209 | 12.832 | 5.568 | 30.442 | 1.00 | 13.14 | L |
| ATOM | 1599 | CD2 | PHE | L | 209 | 13.801 | 4.240 | 28.695 | 1.00 | 15.18 | L |
| ATOM | 1600 | CE1 | PHE | L | 209 | 12.034 | 6.224 | 29.505 | 1.00 | 16.39 | L |
| ATOM | 1601 | CE2 | PHE | L | 209 | 13.005 | 4.890 | 27.751 | 1.00 | 11.07 | L |
| ATOM | 1602 | CZ | PHE | L | 209 | 12.121 | 5.881 | 28.155 | 1.00 | 11.82 | L |
| ATOM | 1603 | C | PHE | L | 209 | 12.473 | 3.173 | 32.287 | 1.00 | 17.65 | L |
| ATOM | 1604 | O | PHE | L | 209 | 11.469 | 2.770 | 31.700 | 1.00 | 16.89 | L |
| ATOM | 1605 | N | ASN | L | 210 | 12.417 | 3.959 | 33.351 | 1.00 | 17.91 | L |
| ATOM | 1606 | CA | ASN | L | 210 | 11.143 | 4.449 | 33.860 | 1.00 | 18.47 | L |
| ATOM | 1607 | CB | ASN | L | 210 | 11.032 | 4.195 | 35.366 | 1.00 | 18.56 | L |
| ATOM | 1608 | CG | ASN | L | 210 | 10.789 | 2.727 | 35.703 | 1.00 | 23.68 | L |
| ATOM | 1609 | OD1 | ASN | L | 210 | 10.902 | 2.320 | 36.860 | 1.00 | 27.80 | L |
| ATOM | 1610 | ND2 | ASN | L | 210 | 10.441 | 1.933 | 34.697 | 1.00 | 20.97 | L |
| ATOM | 1611 | C | ASN | L | 210 | 11.102 | 5.939 | 33.583 | 1.00 | 18.71 | L |
| ATOM | 1612 | O | ASN | L | 210 | 12.073 | 6.647 | 33.842 | 1.00 | 19.41 | L |
| ATOM | 1613 | N | ARG | L | 211 | 9.995 | 6.418 | 33.034 | 1.00 | 18.60 | L |
| ATOM | 1614 | CA | ARG | L | 211 | 9.890 | 7.833 | 32.746 | 1.00 | 19.38 | L |
| ATOM | 1615 | CB | ARG | L | 211 | 8.558 | 8.152 | 32.068 | 1.00 | 19.64 | L |
| ATOM | 1616 | CG | ARG | L | 211 | 8.345 | 9.638 | 31.815 | 1.00 | 19.36 | L |
| ATOM | 1617 | CD | ARG | L | 211 | 6.967 | 9.894 | 31.234 | 1.00 | 20.88 | L |
| ATOM | 1618 | NE | ARG | L | 211 | 5.923 | 9.286 | 32.053 | 1.00 | 21.63 | L |
| ATOM | 1619 | CZ | ARG | L | 211 | 5.540 | 9.733 | 33.245 | 1.00 | 20.71 | L |
| ATOM | 1620 | NH1 | ARG | L | 211 | 6.108 | 10.810 | 33.776 | 1.00 | 19.55 | L |
| ATOM | 1621 | NH2 | ARG | L | 211 | 4.593 | 9.091 | 33.914 | 1.00 | 19.59 | L |
| ATOM | 1622 | C | ARG | L | 211 | 9.981 | 8.592 | 34.058 | 1.00 | 20.65 | L |
| ATOM | 1623 | O | ARG | L | 211 | 9.286 | 8.263 | 35.016 | 1.00 | 21.58 | L |
| ATOM | 1624 | N | GLY | L | 212 | 10.851 | 9.595 | 34.101 | 1.00 | 24.02 | L |
| ATOM | 1625 | CA | GLY | L | 212 | 10.991 | 10.398 | 35.300 | 1.00 | 27.14 | L |
| ATOM | 1626 | C | GLY | L | 212 | 12.033 | 9.932 | 36.301 | 1.00 | 30.84 | L |
| ATOM | 1627 | O | GLY | L | 212 | 12.204 | 10.566 | 37.336 | 1.00 | 32.27 | L |
| ATOM | 1628 | N | ALA | L | 213 | 12.733 | 8.840 | 36.009 | 1.00 | 32.94 | L |
| ATOM | 1629 | CA | ALA | L | 213 | 13.746 | 8.343 | 36.932 | 1.00 | 36.01 | L |
| ATOM | 1630 | CB | ALA | L | 213 | 13.852 | 6.825 | 36.827 | 1.00 | 36.47 | L |
| ATOM | 1631 | C | ALA | L | 213 | 15.101 | 8.979 | 36.657 | 1.00 | 38.12 | L |
| ATOM | 1632 | O | ALA | L | 213 | 15.377 | 9.287 | 35.477 | 1.00 | 39.15 | L |
| ATOM | 1633 | OXT | ALA | L | 213 | 15.875 | 9.147 | 37.627 | 1.00 | 40.06 | L |
| ATOM | 6593 | C | GLY | P | 2 | 10.933 | −14.731 | −32.716 | 1.00 | 43.41 | P |
| ATOM | 6594 | O | GLY | P | 2 | 12.085 | −14.949 | −33.116 | 1.00 | 44.32 | P |
| ATOM | 6595 | N | GLY | P | 2 | 9.495 | −16.601 | −33.373 | 1.00 | 46.06 | P |
| ATOM | 6596 | CA | GLY | P | 2 | 10.040 | −15.847 | −32.211 | 1.00 | 44.91 | P |
| ATOM | 6597 | N | TRP | P | 3 | 10.376 | −13.528 | −32.745 | 1.00 | 40.52 | P |
| ATOM | 6598 | CA | TRP | P | 3 | 11.062 | −12.319 | −33.205 | 1.00 | 35.59 | P |
| ATOM | 6599 | CB | TRP | P | 3 | 10.074 | −11.426 | −33.937 | 1.00 | 35.89 | P |
| ATOM | 6600 | CG | TRP | P | 3 | 9.705 | −11.955 | −35.257 | 1.00 | 37.50 | P |
| ATOM | 6601 | CD2 | TRP | P | 3 | 9.588 | −11.222 | −36.484 | 1.00 | 36.72 | P |
| ATOM | 6602 | CE2 | TRP | P | 3 | 9.180 | −12.147 | −37.469 | 1.00 | 38.08 | P |
| ATOM | 6603 | CE3 | TRP | P | 3 | 9.787 | −9.875 | −36.852 | 1.00 | 37.88 | P |
| ATOM | 6604 | CD1 | TRP | P | 3 | 9.375 | −13.255 | −35.554 | 1.00 | 37.40 | P |
| ATOM | 6605 | NE1 | TRP | P | 3 | 9.060 | −13.374 | −36.873 | 1.00 | 36.98 | P |
| ATOM | 6606 | CZ2 | TRP | P | 3 | 8.964 | −11.778 | −38.788 | 1.00 | 37.40 | P |
| ATOM | 6607 | CZ3 | TRP | P | 3 | 9.571 | −9.515 | −38.166 | 1.00 | 38.81 | P |
| ATOM | 6608 | CH2 | TRP | P | 3 | 9.164 | −10.464 | −39.116 | 1.00 | 38.20 | P |
| ATOM | 6609 | C | TRP | P | 3 | 11.551 | −11.627 | −31.954 | 1.00 | 32.20 | P |
| ATOM | 6610 | O | TRP | P | 3 | 10.824 | −10.841 | −31.348 | 1.00 | 30.08 | P |
| ATOM | 6611 | N | ASN | P | 4 | 12.773 | −11.944 | −31.547 | 1.00 | 28.01 | P |
| ATOM | 6612 | CA | ASN | P | 4 | 13.320 | −11.403 | −30.307 | 1.00 | 24.55 | P |
| ATOM | 6613 | CB | ASN | P | 4 | 14.437 | −12.338 | −29.767 | 1.00 | 24.38 | P |
| ATOM | 6614 | CG | ASN | P | 4 | 14.847 | −12.004 | −28.334 | 1.00 | 26.25 | P |
| ATOM | 6615 | OD1 | ASN | P | 4 | 15.744 | −12.628 | −27.743 | 1.00 | 26.15 | P |
| ATOM | 6616 | ND2 | ASN | P | 4 | 14.176 | −11.015 | −27.766 | 1.00 | 19.93 | P |
| ATOM | 6617 | C | ASN | P | 4 | 13.879 | −10.009 | −30.471 | 1.00 | 22.23 | P |
| ATOM | 6618 | O | ASN | P | 4 | 14.892 | −9.849 | −31.135 | 1.00 | 20.32 | P |
| ATOM | 6619 | N | TRP | P | 5 | 13.235 | −9.008 | −29.875 | 1.00 | 19.93 | P |
| ATOM | 6620 | CA | TRP | P | 5 | 13.693 | −7.622 | −29.964 | 1.00 | 19.18 | P |
| ATOM | 6621 | CB | TRP | P | 5 | 12.826 | −6.747 | −29.044 | 1.00 | 18.29 | P |
| ATOM | 6622 | CG | TRP | P | 5 | 13.181 | −5.288 | −28.984 | 1.00 | 17.73 | P |
| ATOM | 6623 | CD2 | TRP | P | 5 | 14.062 | −4.653 | −28.043 | 1.00 | 16.84 | P |
| ATOM | 6624 | CE2 | TRP | P | 5 | 14.022 | −3.262 | −28.306 | 1.00 | 15.77 | P |
| ATOM | 6625 | CE3 | TRP | P | 5 | 14.879 | −5.123 | −27.002 | 1.00 | 15.75 | P |
| ATOM | 6626 | CD1 | TRP | P | 5 | 12.668 | −4.291 | −29.758 | 1.00 | 17.09 | P |
| ATOM | 6627 | NE1 | TRP | P | 5 | 13.163 | −3.070 | −29.355 | 1.00 | 16.10 | P |
| ATOM | 6628 | CZ2 | TRP | P | 5 | 14.766 | −2.333 | −27.566 | 1.00 | 14.48 | P |
| ATOM | 6629 | CZ3 | TRP | P | 5 | 15.623 | −4.198 | −26.265 | 1.00 | 13.90 | P |
| ATOM | 6630 | CH2 | TRP | P | 5 | 15.558 | −2.815 | −26.554 | 1.00 | 16.26 | P |
| ATOM | 6631 | C | TRP | P | 5 | 15.167 | −7.502 | −29.569 | 1.00 | 20.23 | P |
| ATOM | 6632 | O | TRP | P | 5 | 15.894 | −6.642 | −30.086 | 1.00 | 18.00 | P |
| ATOM | 6633 | N | PHE | P | 6 | 15.602 | −8.357 | −28.643 | 1.00 | 19.67 | P |
| ATOM | 6634 | CA | PHE | P | 6 | 16.988 | −8.333 | −28.177 | 1.00 | 19.01 | P |
| ATOM | 6635 | CB | PHE | P | 6 | 17.137 | −9.138 | −26.871 | 1.00 | 16.46 | P |
| ATOM | 6636 | CG | PHE | P | 6 | 16.558 | −8.448 | −25.665 | 1.00 | 14.70 | P |
| ATOM | 6637 | CD1 | PHE | P | 6 | 15.237 | −8.669 | −25.282 | 1.00 | 13.19 | P |
| ATOM | 6638 | CD2 | PHE | P | 6 | 17.319 | −7.527 | −24.950 | 1.00 | 11.75 | P |
| ATOM | 6639 | CE1 | PHE | P | 6 | 14.686 | −7.982 | −24.208 | 1.00 | 14.31 | P |
| ATOM | 6640 | CE2 | PHE | P | 6 | 16.779 | −6.833 | −23.878 | 1.00 | 13.15 | P |
| ATOM | 6641 | CZ | PHE | P | 6 | 15.461 | −7.056 | −23.503 | 1.00 | 15.00 | P |
| ATOM | 6642 | C | PHE | P | 6 | 17.994 | −8.832 | −29.220 | 1.00 | 19.90 | P |
| ATOM | 6643 | O | PHE | P | 6 | 19.198 | −8.694 | −29.036 | 1.00 | 17.12 | P |
| ATOM | 6644 | N | ASP | P | 7 | 17.500 | −9.405 | −30.315 | 1.00 | 19.83 | P |
| ATOM | 6645 | CA | ASP | P | 7 | 18.390 | −9.887 | −31.372 | 1.00 | 22.24 | P |
| ATOM | 6646 | CB | ASP | P | 7 | 17.922 | −11.239 | −31.925 | 1.00 | 23.36 | P |
| ATOM | 6647 | CG | ASP | P | 7 | 18.021 | −12.357 | −30.908 | 1.00 | 24.59 | P |
| ATOM | 6648 | OD1 | ASP | P | 7 | 18.918 | −12.288 | −30.044 | 1.00 | 26.18 | P |
| ATOM | 6649 | OD2 | ASP | P | 7 | 17.216 | −13.310 | −30.984 | 1.00 | 26.57 | P |
| ATOM | 6650 | C | ASP | P | 7 | 18.477 | −8.905 | −32.534 | 1.00 | 21.96 | P |
| ATOM | 6651 | O | ASP | P | 7 | 19.334 | −9.046 | −33.399 | 1.00 | 21.56 | P |
| ATOM | 6652 | N | ILE | P | 8 | 17.597 | −7.909 | −32.549 | 1.00 | 22.90 | P |
| ATOM | 6653 | CA | ILE | P | 8 | 17.567 | −6.938 | −33.640 | 1.00 | 22.90 | P |
| ATOM | 6654 | CB | ILE | P | 8 | 16.447 | −5.887 | −33.422 | 1.00 | 22.63 | P |
| ATOM | 6655 | CG2 | ILE | P | 8 | 16.511 | −4.812 | −34.510 | 1.00 | 19.27 | P |
| ATOM | 6656 | CG1 | ILE | P | 8 | 15.079 | −6.578 | −33.454 | 1.00 | 22.53 | P |
| ATOM | 6657 | CD1 | ILE | P | 8 | 13.922 | −5.658 | −33.132 | 1.00 | 23.18 | P |
| ATOM | 6658 | C | ILE | P | 8 | 18.881 | −6.212 | −33.941 | 1.00 | 22.27 | P |
| ATOM | 6659 | O | ILE | P | 8 | 19.307 | −6.167 | −35.092 | 1.00 | 24.04 | P |
| ATOM | 6660 | N | THR | P | 9 | 19.526 | −5.653 | −32.925 | 1.00 | 21.64 | P |
| ATOM | 6661 | CA | THR | P | 9 | 20.769 | −4.929 | −33.162 | 1.00 | 22.51 | P |
| ATOM | 6662 | CB | THR | P | 9 | 21.261 | −4.204 | −31.896 | 1.00 | 22.37 | P |
| ATOM | 6663 | OG1 | THR | P | 9 | 21.506 | −5.155 | −30.851 | 1.00 | 20.57 | P |
| ATOM | 6664 | CG2 | THR | P | 9 | 20.218 | −3.179 | −31.438 | 1.00 | 19.23 | P |
| ATOM | 6665 | C | THR | P | 9 | 21.876 | −5.832 | −33.683 | 1.00 | 24.43 | P |
| ATOM | 6666 | O | THR | P | 9 | 22.847 | −5.354 | −34.265 | 1.00 | 25.15 | P |
| ATOM | 6667 | N | ASN | P | 10 | 21.724 | −7.138 | −33.478 | 1.00 | 26.17 | P |
| ATOM | 6668 | CA | ASN | P | 10 | 22.712 | −8.097 | −33.950 | 1.00 | 28.57 | P |
| ATOM | 6669 | CB | ASN | P | 10 | 22.477 | −9.468 | −33.315 | 1.00 | 29.26 | P |
| ATOM | 6670 | CG | ASN | P | 10 | 23.539 | −10.487 | −33.710 | 1.00 | 32.42 | P |
| ATOM | 6671 | OD1 | ASN | P | 10 | 23.221 | −11.623 | −34.049 | 1.00 | 32.26 | P |
| ATOM | 6672 | ND2 | ASN | P | 10 | 24.806 | −10.083 | −33.657 | 1.00 | 30.25 | P |
| ATOM | 6673 | C | ASN | P | 10 | 22.540 | −8.189 | −35.456 | 1.00 | 30.54 | P |
| ATOM | 6674 | O | ASN | P | 10 | 23.512 | −8.162 | −36.209 | 1.00 | 30.18 | P |
| ATOM | 6675 | N | TRP | P | 11 | 21.285 | −8.294 | −35.882 | 1.00 | 32.77 | P |
| ATOM | 6676 | CA | TRP | P | 11 | 20.947 | −8.369 | −37.297 | 1.00 | 34.24 | P |
| ATOM | 6677 | CB | TRP | P | 11 | 19.426 | −8.435 | −37.471 | 1.00 | 34.86 | P |
| ATOM | 6678 | CG | TRP | P | 11 | 18.964 | −7.985 | −38.828 | 1.00 | 37.07 | P |
| ATOM | 6679 | CD2 | TRP | P | 11 | 18.489 | −6.678 | −39.177 | 1.00 | 36.36 | P |
| ATOM | 6680 | CE2 | TRP | P | 11 | 18.227 | −6.684 | −40.565 | 1.00 | 36.61 | P |
| ATOM | 6681 | CE3 | TRP | P | 11 | 18.261 | −5.501 | −38.451 | 1.00 | 35.69 | P |
| ATOM | 6682 | CD1 | TRP | P | 11 | 18.968 | −8.712 | −39.987 | 1.00 | 37.41 | P |
| ATOM | 6683 | NE1 | TRP | P | 11 | 18.527 | −7.936 | −41.034 | 1.00 | 36.44 | P |
| ATOM | 6684 | CZ2 | TRP | P | 11 | 17.748 | −5.557 | −41.242 | 1.00 | 36.21 | P |
| ATOM | 6685 | CZ3 | TRP | P | 11 | 17.786 | −4.379 | −39.126 | 1.00 | 37.21 | P |
| ATOM | 6686 | CH2 | TRP | P | 11 | 17.536 | −4.418 | −40.508 | 1.00 | 37.31 | P |
| ATOM | 6687 | C | TRP | P | 11 | 21.488 | −7.142 | −38.027 | 1.00 | 35.61 | P |
| ATOM | 6688 | O | TRP | P | 11 | 22.121 | −7.263 | −39.074 | 1.00 | 36.68 | P |
| ATOM | 6689 | N | GLY | P | 12 | 21.237 | −5.965 | −37.461 | 1.00 | 34.46 | P |
| ATOM | 6690 | CA | GLY | P | 12 | 21.691 | −4.730 | −38.075 | 1.00 | 36.95 | P |
| ATOM | 6691 | C | GLY | P | 12 | 23.196 | −4.550 | −38.169 | 1.00 | 37.91 | P |
| ATOM | 6692 | O | GLY | P | 12 | 23.692 | −3.987 | −39.144 | 1.00 | 38.24 | P |
| ATOM | 6693 | N | LYS | P | 13 | 23.923 | −5.021 | −37.161 | 1.00 | 39.31 | P |
| ATOM | 6694 | CA | LYS | P | 13 | 25.378 | −4.898 | −37.138 | 1.00 | 40.35 | P |
| ATOM | 6695 | CB | LYS | P | 13 | 25.923 | −5.373 | −35.791 | 1.00 | 40.51 | P |
| ATOM | 6696 | CG | LYS | P | 13 | 27.250 | −4.749 | −35.374 | 1.00 | 42.15 | P |
| ATOM | 6697 | CD | LYS | P | 13 | 28.417 | −5.234 | −36.210 | 1.00 | 44.15 | P |
| ATOM | 6698 | CE | LYS | P | 13 | 29.742 | −4.819 | −35.583 | 1.00 | 45.31 | P |
| ATOM | 6699 | NZ | LYS | P | 13 | 29.877 | −3.339 | −35.455 | 1.00 | 46.91 | P |
| ATOM | 6700 | C | LYS | P | 13 | 25.995 | −5.723 | −38.261 | 1.00 | 41.24 | P |
| ATOM | 6701 | O | LYS | P | 13 | 26.824 | −5.169 | −39.013 | 1.00 | 41.68 | P |
| ATOM | 6702 | OXT | LYS | P | 13 | 25.643 | −6.917 | −38.367 | 1.00 | 41.54 | P |
| END | | | | | | | | | | | |

### Example 2

### Further Structural Analysis

### Fab 4E10 Preparation, Crystallization and Data Collection

Recombinant IgG1(κ) 4E10 was overexpressed in Chinese hamster ovary cells as previously described (Buchacher et al., 1994; Kunert et al., 2000). Antigen-binding fragment Fab 4E10 was obtained by papain digestion of IgG1 4E10. Mercuripapain (Sigma; enzyme at 0.5 mg/ml) was pre-activated with 10 mM cysteine and 1.25 mM EDTA in 0.1 M sodium acetate pH 5.5 for 15 minutes at 37° C. Activated papain solution was then added to IgG1 4E10 (at 5 mg/ml in 0.1 M sodium acetate pH 5.5) to give a final w/w ratio of 4% papain, and the reaction was incubated at 37° C. for 4 hours. Iodoacetamide at a concentration of 20 mM was added and followed by further incubation at 37° C. for 1 hour to stop the digestion reaction.

Fab 4E10 was purified to >95% homogeneity using sequential affinity, size exclusion, and ionic exchange chromatography. Initially, digested sample was diluted 1:3 with 3.0 M NaCl in 0.1 M Tris-HCl pH 9.0 and loaded onto a recombinant protein A column (Repligen). The non-bound material was diluted 1:3 with 10 mM sodium phosphate pH 7.0, 0.15 M NaCl, 10 mM EDTA and loaded onto a recombinant protein G Gammabind Plus column (Amersham Pharmacia). The Fab was eluted using 0.1 M acetic acid, pH 3.0, and immediately neutralized with 1/10 volume of 1.0 M NaHCO₃. The eluted fractions were pooled, dialyzed against 0.2 M sodium acetate pH 5.5, and loaded on a Superdex 75 HR16-60 column (Amersham Pharmacia) equilibrated in 0.2 M sodium acetate pH 5.5. The gel filtrated pooled fractions were further purified by cation exchange chromatography on a MonoS HR5-5 column (Amersham Pharmacia) with 20 mM sodium acetate pH 5.5 and a 0 to 1.0 M NaCl gradient. Pure Fab 4E10 was dialyzed against 20 mM sodium acetate pH 5.5, and concentrated to 12 mg/ml using a Millipore Ultrafree-15 centrifuge concentrator (10 kDa as molecular weight cut-off).

The peptide was synthesized as previously described (Zwick et al., 2001a) and diluted in water to a concentration of 10 mg/ml. Crystals of Fab 4E10 in complex with the peptide were obtained by co-crystallization after overnight preincubation at 4° C. of peptide and Fab 4E10 in a molar ratio of 1:5 (protein:peptide). Crystallization conditions for the complex were initially screened in a nanodrop format (total of 100 nl per drop) using a crystallization robot (Syrrx). Promising crystallization conditions were identified and optimized manually. The best crystals of the complex were grown at 22° C. by sitting drop vapor diffusion against 10-12% (w/v) PEG 8,000 in 0.1 M sodium acetate pH 5.0; 10 mM hexamine cobalt trichloride. Prior to being cooled to cryogenic temperatures, crystals were soaked in a cryoprotectant solution of mother liquor containing 25% (v/v) glycerol. Data were collected on beamline 9-2 at the Stanford Synchrotron Radiation Laboratory (SSRL) using a liquid nitrogen cryostream maintained at 90 K, and processed using the HKL package (Otwinowski and Minor, 1997) and the CCP4 suite of programs (Collaborative Computational Project Number 4, 1994). Diffraction patterns show the contribution of more than one crystalline lattice; however, it was possible to separate and process the diffraction data from only the dominant lattice with good final statistics (Table 2). This crystal belongs to space group C2, with two 4E10-peptide complexes per asymmetric unit (61.5% solvent content and Matthews' coefficient of 3.2 Å³ Da⁻¹). Coordinates and structure factors for Fab 4E10-peptide have been deposited in the Protein Data Bank under accession code 1TZG.

### Structure Determination and Refinement

To examine the interaction of 4E10 with the Trp-rich membrane-proximal region of gp41, the crystal structure of a Fab 4E10-peptide epitope complex was determined at 2.2 Å resolution. The 4E10 epitope is contained within the 13-residue peptide (Lys^{P668} Gly^{P669} TrP^{P670} ASn^{P671} Trp^{P672} Phe^{P673} Asp^{P674} Ile^{P675} Thr^{P676} Asn^{P677} Trp^{P678} Gly^{P679} Lys^{P680}; numbered according to the HXB2 isolate sequence with a P chain identifier) that was previously shown to bind 4E10 (in that study, the peptide was named KGND) (Zwick et al., 2001a). The Lys and Gly residues at either end of the peptide were added to increase peptide solubility in water.

The structure of Fab 4E10 as a complex with the 13-residue peptide was solved by molecular replacement using AMoRe (Navaza, 1994) and Fab 48G7, a catalytic antibody (PDB entry 1HKL), as a probe. The structure was then refined to a resolution of 2.2 Å with R_{cryst}=21.7%, and R_{free}=26.0% (Table 2) in CNS (Brunger et al., 1998) and REFMAC (Collaborative Computational Project Number 4, 1994). R_{free} was calculated using the same set of 5% randomly assigned reflections in both programs. Fab heavy and light chains were treated separately as a rigid body for the initial refinement in CNS. The protein model was then refined using torsion angle simulated annealing at 5,000 K. Following these initial stages, the refinement proceeded through cycles of positional, temperature factor, and manual rebuilding in XFIT (McRee, 1999) into σ_{A}-weighted 2Fₒ-F_{c} and Fₒ-F_{c} electron density omit maps. The maximum likelihood target function, bulk solvent corrections and anisotropic temperature factor corrections were used for the refinement cycles in CNS. Density for the peptide was clear after a few cycles of refinement and manual rebuilding of the starting Fab model. Tight non-crystallographic restraints were used early on in the refinement and released gradually toward the end of the refinement. Water molecules were added automatically using cycles of ARP (Collaborative Computational Project Number 4, 1994) for placement and REFMAC with TLS groups for refinement, then verified by manual inspection in XFIT. Stereochemical analysis of the refined structure was performed using PROCHECK (Collaborative Computational Project Number 4, 1994). Refinement statistics are summarized in Table 2. One of the molecules of the complex in the asymmetric unit (molecule 2) has higher B values (40.4 Å²) than the other (23.3 Å²) due to fewer crystal packing contacts.

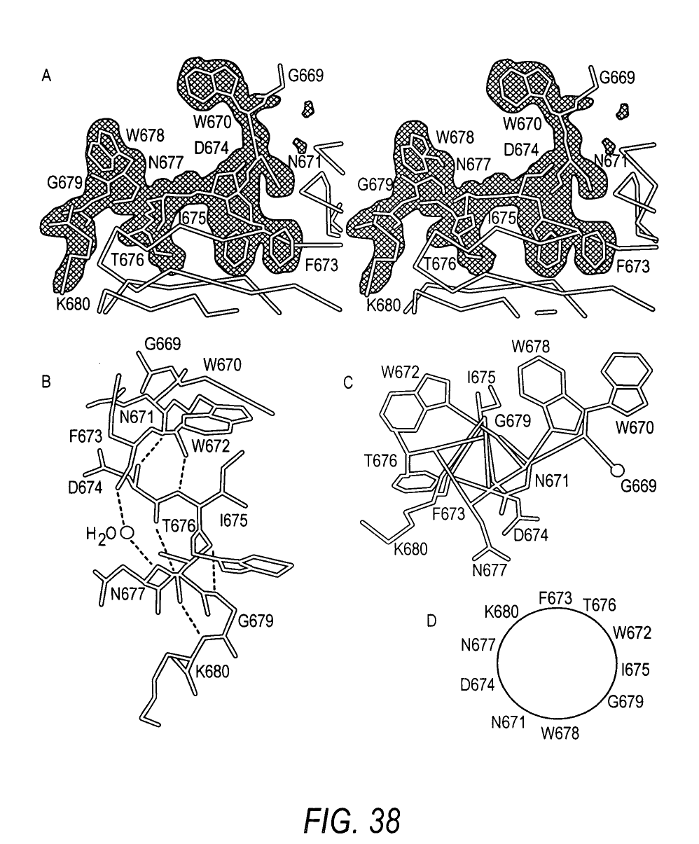

The final model contains Fab residues L1-L212, H1-H232 (Fab residues are numbered according to standard convention (Kabat et al., 1991) with light and heavy chain identifiers L and H, respectively) and peptide residues P669-P680. Heavy chain C-terminal residues (Ser^{H229}, Cys^{H230}, Asp^{H231}, and Lys^{H232}) were visible in one Fab (molecule 1). Electron density omit maps clearly defined the location and conformation of the peptide in the binding site of 4E10 (FIG. 38A). The only peptide residue with no interpretable electron density is the N-terminal Lys^{P668}, which was omitted from the model. FIG. 38 depicts the structure of the peptide bound to Fab 4E10, in this case, the peptide sequence is KGWNWFDITNWGK (SEQ ID NO: 2) and it encompasses the 4E10 epitope. FIG. 38A provides a stereo view of the peptide structure superimposed on the sigma A-weighted Fo-Fc electron density omit map contoured at 46. Clear density is evident for all peptide residues except at the N-terminus. Part of the heavy (gray) and light (pink) chains of the antibody are displayed. FIGS. 38B and 38C provide the side and top views, respectively, of the peptide helix. Hydrogen bonds involved in stabilization of the helical conformation are shown as dotted lines. FIG. 38D is a representation of the peptide helical wheel. The residues in the polar face are in red.

The Fab 4E10-peptide complex model has good geometry with only Ala^{L51}, which is in a conserved γ turn as observed in most antibody structures (Stanfield et al., 1999), in the disallowed region of the Ramachandran plot (Table 2). The two molecules in the asymmetric unit are similar, whereas individually the C_{α}'s of peptide residues, constant or variable Fab domains superimpose with r.m.s. deviations below 0.4 Å. Thus, only the complex with lower B values (molecule 1) is described here.

### Structural Analysis

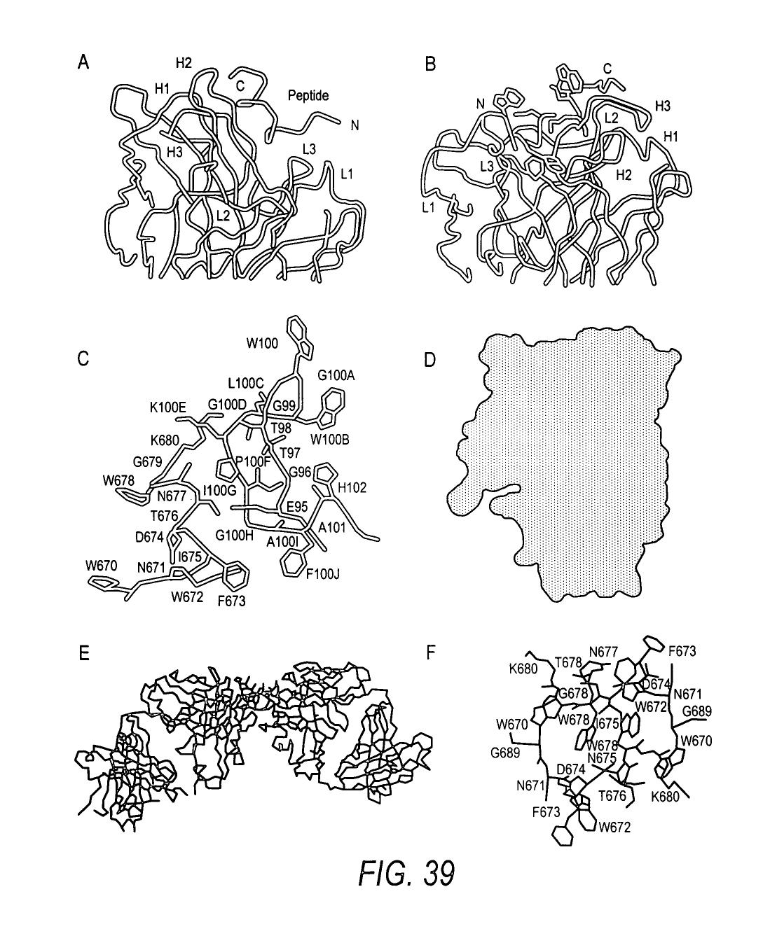

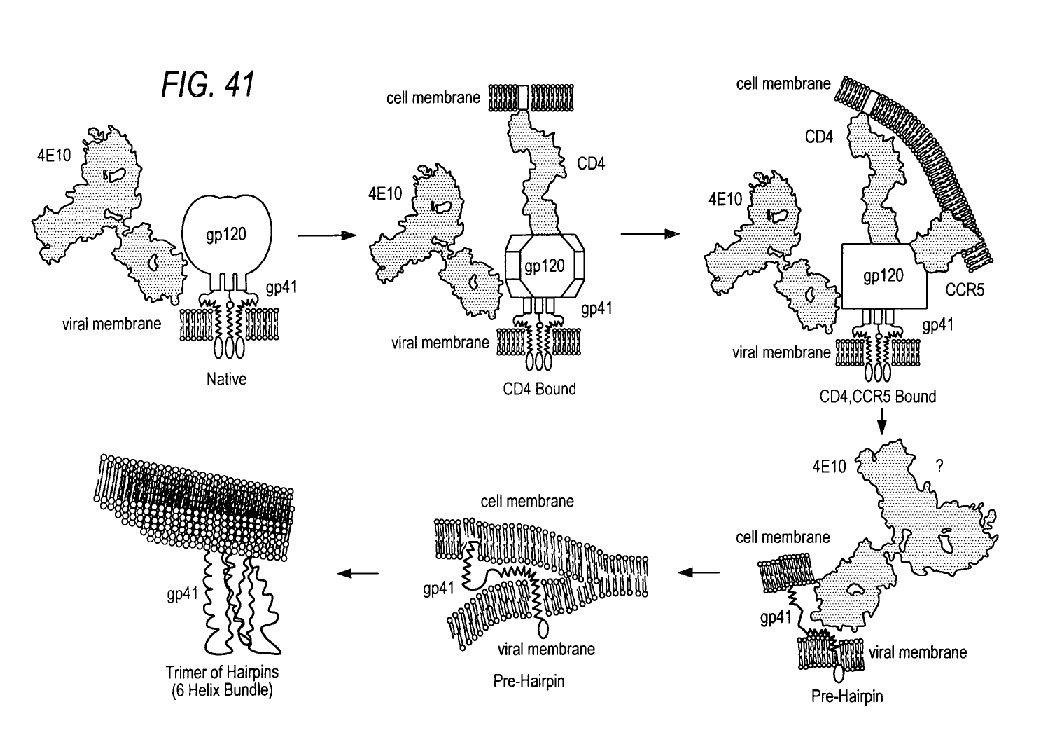

Superpositions and root mean square deviations (r.m.s.d.) calculations were carried out using the INSIGHT II package (Accelrys, Inc., San Diego, Calif.) for pairs of C_{H}, C_{L}, V_{H}, and V_{L} domains. Hydrogen bonds between Fab 4E10 and peptide were identified using HBPLUS (McDonald and Thornton, 1994) and van der Waals contacts were assigned with CONTACSYM (Sheriff et al., 1987). Buried surface areas were calculated using MS (Connolly, 1993) with a 1.7 Å probe radius and standard van der Waals radii (Gelin and Karplus, 1979). The Lys^{P680} to Trp^{P680} change was modeled with XFIT (McRee, 1999). Secondary structure was assigned using PROMOTIF (Hutchinson and Thornton, 1996). Graphics were prepared using XFIT (FIGS. 38, 39E, and 39F), RASTER3D (Merritt and Bacon, 1997) (FIGS. 38-40), GRASP (Nicholls et al., 1991) (FIG. 39D), MOLSCRIPT (Kraulis, 1991) (FIGS. 39A-39D and 40), and MODELZILLA (FIG. 41).

FIG. 39 depicts the antigen binding site of Fab 4E10. FIGS. 39A and 39B show the CDRs L1, L2, L3, H1, H2, and H3 highlighted in the Fab 4E10-peptide complex: the light chain (pink) CDRs L1 (dark blue) and L3 (green) and the heavy chain (gray) CDRs H1 (orange), H2 (magenta), and H3 (red) bind the peptide (yellow). CDR L2 (cyan) does not contact antigen. FIG. 39C shows the conformation of the H3 loop in the peptide-bound structure of Fab 4E10. The H3 loop (gray backbone with pink side chains) is rich in Gly and Trp residues. The peptide (yellow) is shown for reference. FIG. 39D depicts the electrostatic potential surface of Fab 4E10 with a bound peptide. Negatively-charged regions are red, positively charged regions are blue, and neutral regions are white (±15 kV potential range). The peptide (yellow) binds to a shallow hydrophobic cavity on the antibody. FIG. 39E shows an overall view of two molecules of the Fab 4E10-peptide complex in the unit cell. The crystal contacts in this region are close to the antigen binding site of Fab 4E10 (heavy chains are gray and green; light chains are salmon and blue). The peptides (yellow and purple chains) are located in the interface between the two related Fab molecules. FIG. 39F depicts the interaction of two peptide chains in the unit cell show the close interdigitation of their indole side chains.

FIG. 40 depicts contacts between Fab 4E10 and key residues of its epitope. Hydrogen bonds are shown as dotted lines. Light, heavy, and peptide chains are shown in pink, gray, and yellow, respectively. FIG. 40A shows contacts between Fab 4E10 and peptide residues Trp^{P672} and Phe^{P673}. FIG. 40B shows contacts between Fab 4E10 and peptide residues Ile^{P675} and Thr^{P676}. FIG. 40C shows contacts between Fab 4E10 and peptide residues Lys^{P680} and modeled Trp^{P680} (green). The side chain of Trp^{P672} is shown in 40B and 40C for reference.

Fab 4E10 has the canonical β-sandwich immunoglobulin fold with an elbow angle of 193° for both molecules in the asymmetric unit. The complementarity determining regions (CDRs), or hypervariable loops, L1, L2, L3, H1, and H2 belong to canonical classes 2, 1, 1, 1, and 2, respectively, as determined from the length, sequence, and conformation of the loops (A1-Lazikani et al., 1997) (FIGS. 39A and 39B). CDR H3 bends away from the binding site to allow interaction of its base and central residues with the C-terminal region of the peptide (FIG. 39B).

Antibody 4E10 has a long CDR H3 (Glu^{H95} Gly^{H96} Thr^{H97} Thr^{H98} Gly^{H99} Trp^{H100} Gly^{H100A} Trp^{H100B} Ile^{H100C} Gly^{H100D} Lys^{H100E} Pro^{H100F} Ile^{H100G} Gly^{H100H} Ala^{H100I} Phe^{H100J} Ala^{H101} His^{H102}) with a ten amino acid insert after residue 100. Such long CDR H3 loops are also found in other HIV-1 MAbs, such as 2F5 (Barbato et al., 2003), Z13 (Zwick et al., 2001a), b12 (Saphire et al., 2001), 447-52D (Stanfield et al., 2004), and 17b (Kwong et al., 1998) and may facilitate access to concave or relatively inaccessible sites. In addition, the H3 loop of 4E10 is quite hydrophobic and rich in Gly and Trp residues (FIG. 39C); five Gly and two Trp residues are present in the 18 residues of the H3 loop. The Gly residues give the loop some conformational freedom, while the Trp residues may facilitate interactions with hydrophobic regions in or around the membrane-proximal region of gp41, including the viral membrane (Ofek et al. Manuscript in preparation). Thus, the size and amino acid composition of the H3 loop may facilitate 4E10 access and binding to its partially occluded epitope in the native gp41 oligomer.

The 13-residue peptide is bound to Fab 4E10 in a helical conformation (FIGS. 38 and 39) as found for a 19-residue peptide (KWASLWNWFNITNWLWYIK (SEQ ID NO: 1); residues 665-683 of the Trp-rich membrane-proximal region of gp41) in membrane-mimetic dodecylphosphocholine micelles by NMR spectroscopy (Schibli et al., 2001). The 13-residue peptide has an α-helical conformation from Asp^{P674} to Lys^{P680} preceded by a short 3₁₀ helix (Asn^{P671} and Trp^{P672}) and an extended structure (Gly^{P669} and)Trp^{P670}) at the N-terminus (FIGS. 38B and 38C). The transition from 3₁₀ helix to α-helix occurs at Phe^{P673}, where the carbonyl oxygen makes a water-mediated hydrogen bond to the backbone nitrogen of Asn^{P677} (FIG. 38B), the i+4 residue from Phe^{P673}, in an almost α-helical manner. The 3₁₀ helix has been suggested to act as a folding intermediate in α-helix formation. The helical conformation creates an amphipathic structure with a narrow polar face (defined by residues Asn^{P671}, Asp^{P674}, Asn^{P677}, and)Lys^{P680} and a hydrophobic face (Trp^{P672}, Phe^{P673}, Ile^{P675}, Thr^{P676}, Trp^{P678}, and Gly^{P679}) (FIGS. 38C, 38D, 39C and 39D). Residue Lys^{P680}, which is part of a solubility tag, corresponds to the universally-conserved Trp in the gp41 sequence and is located between the two faces. In addition, the H3 loop of 4E10 is quite hydrophobic and rich in Gly (5) and Trp (2) residues (FIG. 39c). The Gly residues give the loop some conformational freedom, while the Trp residues may facilitate interactions with hydrophobic regions in or around the membrane-proximal region of gp41, including the viral membrane (Ofek, submitted). The Fab-bound peptide structure thus defines the minimal 4E10 epitope as WFXYZ, where X does not play a major role in 4E10 binding, Y can be Ile/Leu/Val, and Z can be Thr/Ser. The WFXYZ motif appears to be absolutely conserved in all HIV-1 viruses. The remarkable broadly neutralizing activity of 4E10 appears to derive from its ability to recognize the most conserved gp41 residues within its core epitope sequence. The majority of the contacts (36%) are made with the absolutely-conserved Trp672 of gp41.

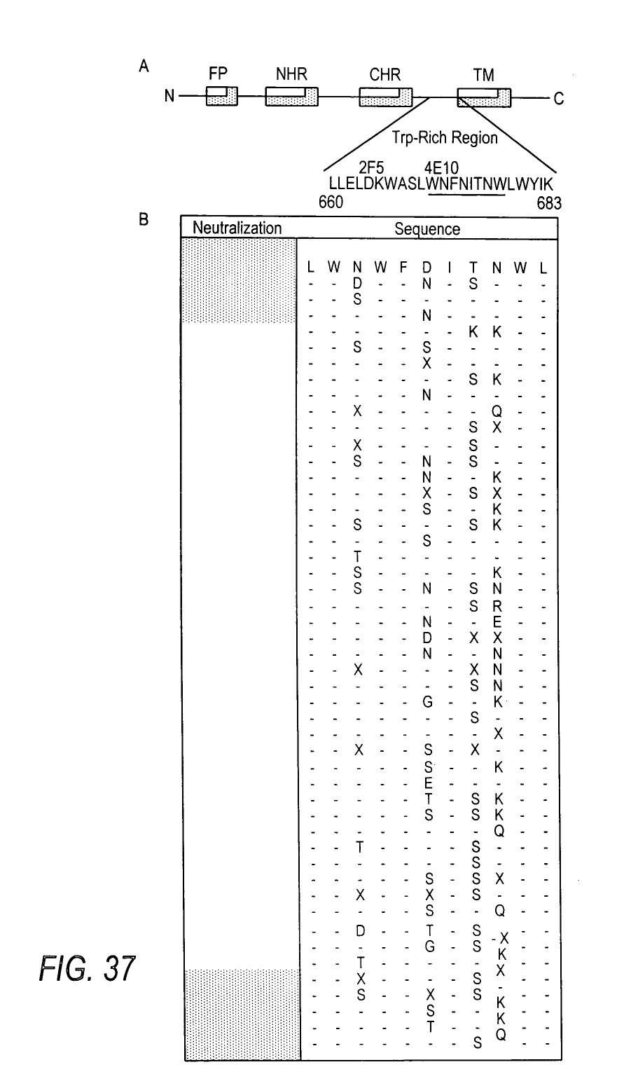

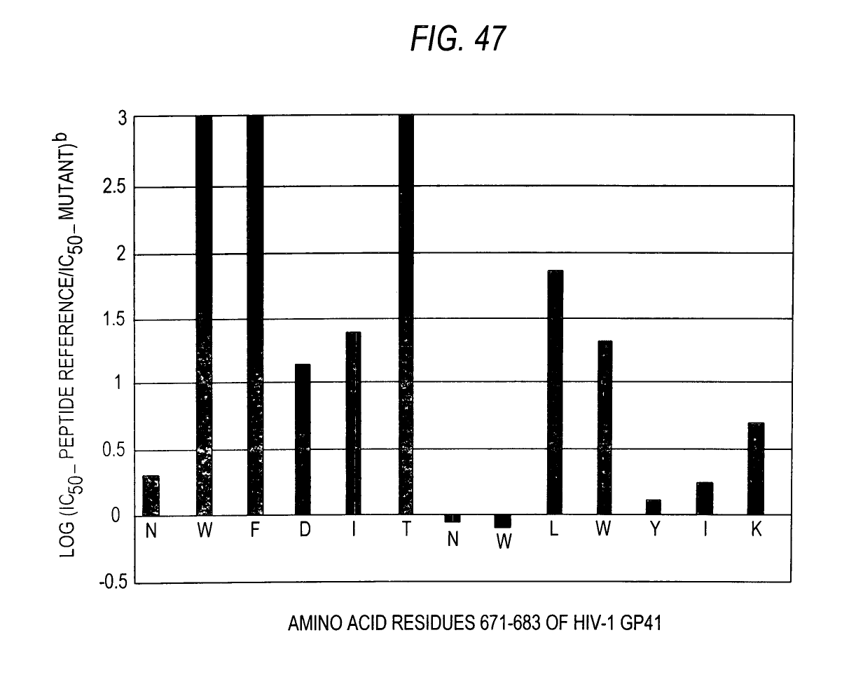

FIG. 47 depicts both the schemiatic representation of gp41 and the neutralizing activity of 4E10. FIG. 47a shows important functional regions include the fusion peptide (FP; purple box), the N- and C-terminal heptad repeat regions (NHR, green box, and CHR, red box, respectively), and the transmembrane region (TM; yellow box). The location and sequence of the Trp-rich region are indicated with the core 2F5 and 4E10 epitopes shown in red and the region contained within the peptide used in this study underlined. Sequence numbering follows strain HXB2. The various domains are not drawn to scale. FIG. 37b, depicts the neutralizing activity of 4E10 against a panel of viruses from different clades. A total of 93 viruses were analyzed of which 52 have unique sequences in the 4E10 epitope region shown here. The sequences are arranged in order of neutralization sensitivity from the most sensitive (red; IC₅₀<1 μg/mL) to the most resistant (green; IC₅₀>50 μg/mL. The intermediate sensitivity, 1 μg/mL>IC₅₀>50 μg/mL, is in yellow). The sequences around the 4E10 epitope are shown with conserved residues as dashes.

In complexes between peptides and anti-peptide antibodies, β-turns are the predominant secondary structure of the bound peptide (Stanfield and Wilson, 1995). Thus, the conformation of the peptide bound to 4E10 is highly unusual. Helical peptides bound to antibody have rarely been reported. To date, only two other examples of crystal structures of complexes between helical peptides and antibodies have been deposited in the Protein Data Bank: an anti-interleukin 2 Fab in complex with an antigenic nonapeptide with 7 residues in an α-helical conformation (PDB access code 1F90) (Afonin et al., 2001), and antibody C21 in complex with its epitope on P-glycoprotein where all 11 peptide residues form an α-helix (PDB code 2AP2) (van Den Elsen et al., 1999).

### Binding Affinity by ELISA

Enzyme-linked immunosorbent assays (ELISA) were used to determine the binding affinity of the antibody for the peptide and gp41. Microplate wells (Corning) were coated overnight at 4° C. with 50 μl of PBS containing peptide (4.1 μg/ml) or recombinant gp41 (4 μg/ml). The wells were washed twice with PBS containing 0.05% Tween 20 and blocked with 3% BSA for 45 min at 37° C. After a single wash, 4E10 (5 μg/ml) was added to the wells in PBS containing 1% BSA and 0.02% Tween and allowed to incubate at 37° C. for 2 h. The wells were washed four times, goat anti-human IgG F(ab′)₂ alkaline phosphatase (Pierce) diluted 1:500 in PBS containing 1% BSA was added, and the plate was incubated for 40 min at room temperature. The wells were washed four times and developed by adding 50 μl of alkaline phosphatase substrate, prepared by adding one tablet of disodium-p-nitrophenyl phosphate (Sigma) to 5 ml of alkaline phosphatase staining buffer (pH 9.8), as specified by the manufacturer. After 30 min, the optical density at 405 nm was read on a microplate reader (Molecular Devices).

Antibody 4E10 binds with approximately 4-fold higher affinity to recombinant gp41 than to the synthetic peptide (data not shown), as determined by enzyme-linked immunosorbent assays (ELISA). The reduced affinity of 4E10 for the peptide could be due to lack of appropriate flanking residues or conformational restraints of the peptide conformation in gp41. Nevertheless, the contact residues between 4E10 and the core epitope are likely to be the same on gp41.

### Structural Basis for 4E10 Specificity

Specific antibody-antigen recognition comes from steric and chemical complementarity between antigen and antibody. The Fab 4E10 combining site is mostly a hydrophobic cavity (FIG. 39D) that allows a close fit of the amphipathic peptide. The antibody surface area buried by the peptide is approximately 580 Å², whereas the corresponding area on the peptide is about 529 Å². Although these values are comparable to those found in other Fab-peptide complexes (Stanfield and Wilson, 1995), the 4E10 peptide additionally buries an extra 360 Å² of its surface due to crystal packing. In the crystal, two peptide molecules are related by a 2-fold symmetry axis and are adjacent to each other (FIGS. 39E and 39F). This supersecondary interaction of the two peptide chains (FIG. 39F) combines to bury the hydrophobic peptide almost completely and perhaps mimics the low-energy conformation in the intact gp41 oligomer or the association with the viral membrane.

Fab 4E10 uses five of its six CDR loops to bind the peptide; CDR L2 is not used and CDR L1 makes only minor contacts (FIG. 39B). Eight hydrogen bonds, 1 salt bridge, and 98 van der Waals contacts are made between peptide and Fab residues from CDRs L1 (4% of total contacts), L3 (28%), H1 (8%), H2 (41%), and H3 (19%) (Table 3). Ten additional hydrogen bonds between peptide and Fab residues are mediated by water molecules buried at the Fab-peptide interface.

The extent and nature of the Fab-peptide interactions define the relative importance of each peptide residue for complex formation. In a helical conformation, the peptide backbone cannot easily engage in hydrogen bonds to the Fab because of the intra-peptide hydrogen bonding along the helix. The peptide recognition then depends mainly on interactions in which the peptide side chain knobs from the helix intercalate into holes on the antibody surface. The helical conformation of the bound peptide places the side chains of Trp^{P672} and Phe^{P673} on the same side of the peptide and along with Ile^{P675}, Thr^{P676}, and Lys^{P680} forms an extensive hydrophobic face that intimately contacts the Fab (FIGS. 38 and 39). The side chains of Trp^{P672} and Phe^{P673} insert into a pocket in the antibody-combining site, where they form a cluster of aromatic rings with Fab residues Tyr^{L91}, Trp^{H47}, and Phe^{H100J} (FIG. 40A). In addition to the 37 van der Waals contacts, the main chain and side chain of Trp^{P672} hydrogen bond to Ser^{L94} and Ile^{H56}, respectively (Table 3 and FIG. 40A). The Trp^{P672} contacts represent 36% of the total contacts between Fab 4E10 and peptide that make it the most important residue in the antibody-peptide interaction (Table 3); the majority of these contacts (85%) are with CDR H2 (residues Gly^{H50}, Val^{H51}, Ile^{H52}, Ile^{H56}, and Asn^{H58}). The next key peptide residues are Thr^{P676} and Phe^{P673}, which make 18% and 14% of the total contacts with the Fab, respectively. Phe^{P673} works cooperatively with Trp^{P672} to form the cluster of aromatic rings in the binding site (FIG. 40A). In addition to several van der Waals contacts, the side chain of Thr^{P676} hydrogen bonds to the carboxyl of Glu^{H95} (Table 3 and FIG. 40B). Thr^{P676} along with Lys^{P680} are the peptide residues with the most interactions with the H3 loop (Table 3). Even though Ile^{P675} is responsible for only 6% of the contacts between 4E10 and the peptide, the side chain of Ile^{P675} stacks with the side chains of Ile^{H52} and Ile^{H56} to create a small cluster of isoleucines on the edge of the antibody-combining site (FIG. 40B).

Mutagenesis of HIV-1 has recently shown that Trp^{P680} is important for 4E10 neutralization (Zwick. et al. Manuscript in preparation). In the peptide used here, a Lys rather than a Trp was substituted at position 680 to increase peptide solubility. To explore the structural role of Trp^{P680} in the binding site, Trp^{P680} was modeled in place of Lys^{P680} in an orientation that maximizes contacts with 4E10 (FIG. 40C). In this conformation, the Nₑ₁ atom of Trp^{P680} would hydrogen bond to the carbonyl oxygen of Leu^{H100C}, in the same way as the N_{ξ} atom of Lys^{P680} hydrogen bonds to Leu^{H100C} in the crystal structure. In addition, Trp^{P680} would pack with Tyr^{H32} and Pro^{H100F} (FIG. 40C) forming a second cluster of aromatic residues in the antibody-combining site. All of these proposed contacts would place Trp^{P680}, together with Trp^{P672}, Phe^{P673}, Ile^{P675}, and Thr^{P676}, as a critical residue for 4E10 specificity for gp41.

### Discussion

The structural analysis of the contributions made by each peptide residue to 4E10 binding reveals the key epitope residues and complements results obtained from epitope mapping (Zwick et al., 2001a) and mutagenesis experiments (Zwick et al. Manuscript in preparation). Previously, 4E10 was mapped to a linear epitope comprising residues NWF(D/N)IT (SEQ ID NO: 77) (Zwick et al., 2001a) on the 671-679 Trp-rich region of gp41. The crystal structure of the Fab 4E10-epitope complex illustrates that Trp^{P672}, Phe^{P673}, Ile^{P675}, and Thr^{P676} make the greatest number of selective contacts with 4E10. These peptide residues dictate 4E10's high affinity for the epitope. Trp^{P672}, Phe^{P673} (and probably Trp^{P680}; a Lys was present at this position in the peptide used here) side chains are buried in the binding site and are involved in aromatic 7π-stacking interactions. The most important residue for antibody-peptide binding is Trp^{P672}, which alone is responsible for 36% of the total contacts between the Fab and the peptide. In comparison, Ile^{P675} and Thr^{P676} have a secondary role for defining the 4E10 specificity. Thr^{P676} can be replaced by a serine without affecting 4E10 binding and Ser is found in many HIV isolates that are neutralized by 4E10. Such Thr/Ser change can maintain the hydrogen bond with CDR H3 residue Glu^{H95}. On the other hand, Ile^{P675}, which is highly conserved and forms part of a cluster of three isoleucines in the binding site, is not involved in as many contacts with 4E10 and can be replaced by other medium-size hydrophobic residues, such as Leu or Val, without any drastic decrease in 4E10 affinity for gp41. Thus, the minimal epitope for 4E10 can now be defined as WFXYZ, where X does not play a major role for 4E10 binding, Y can be Ile/Leu/Val, and Z can be Thr/Ser. Since the X residue must not make steric clashes with the antibody binding site, some restrictions about the size and chemical features of this side chain still remains.

The 4E10 epitope is part of the fusion machinery of HIV and Trp⁶⁷² has a crucial role in virus infectivity (Salzwedel et al., 1999). Second, the variable residues that flank the conserved Trp^{P672}, Phe^{P673}, Ile^{P675}, and Thr/Ser^{P676} are located on the opposite side of the helical epitope and are not involved in many contacts with the antibody. These variable residues might be masked in the interface of a gp41 oligomer or embedded in the viral membrane.

Although HIV-1 entry into human cells has been extensively investigated, many aspects of the process remain undefined. It is hypothesized that before CD4 binding, gp41 is in a metastable conformation with the fusion peptide buried in the gp41 structure (Gallo et al., 2003) (FIG. 41). FIG. 41 is a cartoon representation of a hypothetical model of HIV env-mediated membrane fusion and virus neutralization by antibody 4E10. The native state of the gp120-gp41 complex is metastable and triggered by gp120 binding to CD4 and coreceptor (here CCR5). The 4E10 epitope on gp41 is represented as a pink helix parallel to the plane of the viral membrane and the epitope seems to be exposed and susceptible to antibody binding and virus neutralization in the metastable and receptor-bound states of gp41. Conformational changes of the Env proteins leading to the pre-hairpin intermediate cause gp120 dissociation of gp41 and insertion of the gp41 fusion peptide into the host cell membrane. For clarity, only one gp41 monomer is shown for the pre-hairpin state (N-terminal heptad repeat is a pink helix and C-terminal heptad repeat is a green helix). 4E10 binding to the extended pre-hairpin intermediate is a possibility to be still proved. The viral and cell membranes are brought into close proximity and the orientation of the helical gp41 membrane-proximal region parallel to the membranes with the Trp residues around the helix axis could aid in the disruption of both membranes. In the final stages of fusion, the C-terminal heptad repeat folds back onto the N-terminal heptad repeat to generate a trimer of hairpins also known as the 6-helix bundle structure.

Binding of gp120 to CD4 and coreceptor (CCR5 or CXCR4) triggers conformational changes in gp120 and gp41, resulting in dissociation of gp120 from gp41 and change of gp41 to a pre-hairpin intermediate conformation in which the fusion peptide is inserted into the host membrane and the N- and C-terminal heptad repeat regions are separated (Gallo et al., 2003). The C-terminal heptad repeat region would then fold back onto the N-terminal heptad repeat to generate a trimer of hairpins (also known as the six-helix bundle) with the three C-terminal helices wrapped around the central three N-helices in an antiparallel orientation (Weissenhorn et al., 1997; Chan et al., 1997). Transition from the pre-hairpin to the hairpin gp41 structure brings the host and viral membranes into close proximity. The Trp-rich region of gp41 may be or become parallel to the plane of the viral-host membranes and the distribution of Trp residues around the helix could then allow the Trp-rich region to disrupt both membranes (Schibli et al., 2001), and aid in the formation of a fusion pore along with the fusion peptide. The binding of 4E10 to the Trp-rich region would prevent such an event. The final step of the fusion process is pore expansion to a size that permits passage of the viral nucleocapsid. A cluster of several HIV Env trimers must interact with a cluster of host cell receptors for the fusion process take place efficiently.

The membrane-proximal region of gp41 appears to be quite flexible and apparently changes conformation during the course of the membrane fusion event. The membrane-proximal region is suggested to first extend and then contract to a helical structure (Barbato et al., 2003). Such a structural transition is in agreement with data showing the region in a mostly extended conformation with a central Asp⁶⁶⁴-Lys⁶⁶⁵-Trp⁶⁶⁶ β-turn when bound to MAb 2F5 (Barbato et al., 2003), as a 3₁₀ helix in water (Biron et al., 2002), and as an α-helix in a membrane-mimic micelle (Schibli et al., 2001) and when bound to 4E10 (this study). The 3₁₀ helix could be an intermediate to the final α-helix. The 4E10 epitope region might be helical all or most of the time since it is very close to the helical transmembrane domain and has been shown to be exposed and susceptible to antibody binding and virus neutralization by 4E10, at least when gp41 is in the native metastable and receptor-bound conformations (Binley et al., 2003) (FIG. 41). In addition, the 4E10 epitope could still be accessible when gp41 is in the extended pre-hairpin conformation. However, 4E10 binding to the extended pre-hairpin intermediate has still to be proved. In the metastable and receptor-bound conformations, 4E10 epitope may be partially occluded by the gp120-gp41 oligomer. If at this stage, the Trp-rich helix is already parallel to the membrane, as suggested from the NMR structure of this region in a membrane-mimic micelle (Schibli et al., 2001) and as represented in FIG. 41, the 4E10 epitope might be less occluded by the gp120-gp41 oligomer than if the region is perpendicular to the membrane and is part of a gp41 oligomer. In either of these scenarios, the size and hydrophobic character of the CDR H3 of 4E10 should be an important feature to facilitate interaction with the partially occluded and membrane-proximal 4E10 epitope. The five Gly residues may give the CDR H3 conformational freedom and eliminate potential steric clashes with side chains. The H3 loop size and flexibility would allow a potential interaction between the tip of the loop (Pro^{H100F}) and Trp⁶⁸⁰, a gp41 residue located only a few residues further from the membrane (FIG. 40C). Simultaneously, the two Trp residues located close to the tip of the H3 loop (Trp^{H100} and Trp^{H100B}) (FIG. 39C) have the potential to enhance the interaction between 4E10 and HIV by inserting their side chains into the viral membrane when the tip of the H3 loop is contacting the epitope, similarly to that proposed for 2F5 (Ofek et al. Manuscript in preparation). Mutagenesis studies of the H3 loop of 4E10 are ongoing to test the importance of the CDR H3 for 4E10 binding to gp41 in virus particles.

The fact that the 4E10 epitope is contiguous and highly conserved among HIV isolates of different clades makes the epitope a good lead for structure-based design of a broadly effective HIV-1 vaccine. 4E10 may also increase the efficacy of an antibody combination therapy, since 4E10 neutralizes viruses that are not neutralized by other available MAbs. Despite the contiguous nature of the 4E10 epitope, denaturation of recombinant gp41 reduces the binding of 4E10, but not of 2F5 (Zwick et al., 2001a). This effect suggests the importance of the helical epitope conformation for MAb 4E10. The 13-residue peptide used in this study therefore mimics the biologically-relevant conformation of its cognate epitope on gp41 and helical peptide analogs could be used to focus the immune response to induce higher titers of 4E10-like antibodies able to neutralize a broad range of HIV subtypes.

TABLE 2
X-ray Diffraction Data and Refinement Statistics for the Complex
Crystal Features

| | |
|---|---|
| Space group | C2 |
| No. of molecules of complex per asym. | 2 |
| unit | |
| Unit cell parameters (Å, °) | A = 157.3, b = 45.1, c = 198.5, |
| | β = 113.8 |

Data Quality

| | |
|---|---|
| Resolution (Å)^{a} | 50.00-2.20 (2.28-2.20) |
| No. of observations | 198,794 |
| No. of unique reflections | 61,572 |
| Mosaicity (°) | 0.35 |
| Completeness (%)^{a} | 93.0 (61.4) |
| Multiplicity^{a} | 3.2 (2.2) |
| I/σ (I)^{a} | 16.7 (2.3) |
| R_{sym} (%)^{a,b} | 7.5 (37.1) |

Model Quality

| | |
|---|---|
| R_{cryst} (%)^{c} | 21.7 |
| R_{free} (%)^{c} | 26.0 |
| No. of protein atoms | 6907 |
| NO. OF WATER | 612 |
| MOLECULES | |
| Average B value (Å²) | 22.2, 19.5, 28.3 |
| Molecule 1 (Heavy, Light, Peptide) | 41.0, 46.5, 33.8 |
| Molecule 2 (Heavy, Light, Peptide) | 36.2 |
| A. Water molecules | 0.005 |
| R.m.s deviation for bond lengths (Å) | 1.3 |
| R.m.s deviation for bond angles (°) | |
| Ramachandran Plot | 87.2 |
| Most favored regions (%) | 12.4 |
| Additional allowed regions (%) | 0.1 |
| Generously allowed regions (%) | 0.3^{d} |
| Disallowed regions (%) | |

^{a}Values in parentheses correspond to the highest resolution shell.
^{b}R_{sym} = [ΣₕΣᵢ|Iᵢ(h) − <I(h)>|/ΣₕΣᵢIᵢ(h)] × 100, where <I(h)> is the mean of the I(h) observation of reflection i.
^{c}R = Σₕₖₗ|Fₒ − F_{c}|/Σₕₖₗ|Fₒ|. R_{free} was calculated as R but, using only 5% of data reserved for the cross-validation.
^{d}the only residue present in the disallowed region is Ala^{L51}, which is in a conserved γ turn as observed in most antibody structures.

TABLE 3
Direct Contacts Between Fab 4E10 and Peptide
van der Waals contacts

| Peptide residue | Fab 4E10 residue |
|---|---|
| Asn^{P671} | Gly^{L92}, Gln^{L93}, Ser^{L94} |
| Trp^{P672} | Ser^{L94}, Ala^{H33}, Gly^{H50}, Val^{H51}, Ile^{H52}, Ile^{H56}, Asn^{H58} |
| Phe^{P673} | Tyr^{L91}, Ser^{L94}, Trp^{H47}, Phe^{H100J} |
| Asp^{P674} | Lys^{L32} |
| Ile^{P675} | Ile^{H52}, Ile^{H56} |
| Thr^{P676} | Thr^{H31}, Tyr^{H32}, Ala^{H33}, Ile^{H52}, Glu^{H95}, Pro^{H100F} |
| Asn^{P677} | Pro^{H100F} |
| Lys^{P680} | Leu^{H100C}, Gly^{H100D}, Pro^{H100F} |

Hydrogen bond and salt bridge contacts

| Peptide atom | Fab 4E10 atom | Distance (Å) |
|---|---|---|
| Trp^{P670}-O | Ser^{L94}-O_{γ} | 3.4 |
| Asn^{P671}-O_{δ1} | Tyr^{L91}-O | 2.9 |
| Asn^{P671}-N_{δ2} | Ser^{L94}-N | 3.2 |
| Trp^{P672}-N | Ser^{L94}-O_{γ} | 3.2 |
| Trp^{P672}-N_{ε1} | Ile^{H56}-O | 3.2 |
| Asp^{P674}-O_{δ1} | Lys^{L32}-N_{ξ} | 3.4 |
| Thr^{P676}-O_{γ1} | Glu^{H95}-O_{ε1} | 3.0 |
| Thr^{P676}-O_{γ1} | Glu^{H95}-O_{ε2} | 2.8 |
| Lys^{P680}-N_{ξ} | Leu^{H100C}-O | 2.7 |

### Example 3

### Development of Peptides and Peptidomimetics

As previously described, the structures of the 4E10 and 2F5 peptide epitopes have been analyzed. These structures provide insight into the conformations that compounds have to adopt in order to elicit neutralizing antibodies. 4E10 is the most broadly neutralizing HIV-1 Mab known, and recognizes a highly conserved, contiguous helical epitope in the gp41 membrane proximal region. Based on the crystal structure of the 4E10/epitope peptide complex, helical peptides and small molecule helix mimics are developed as immunogens.

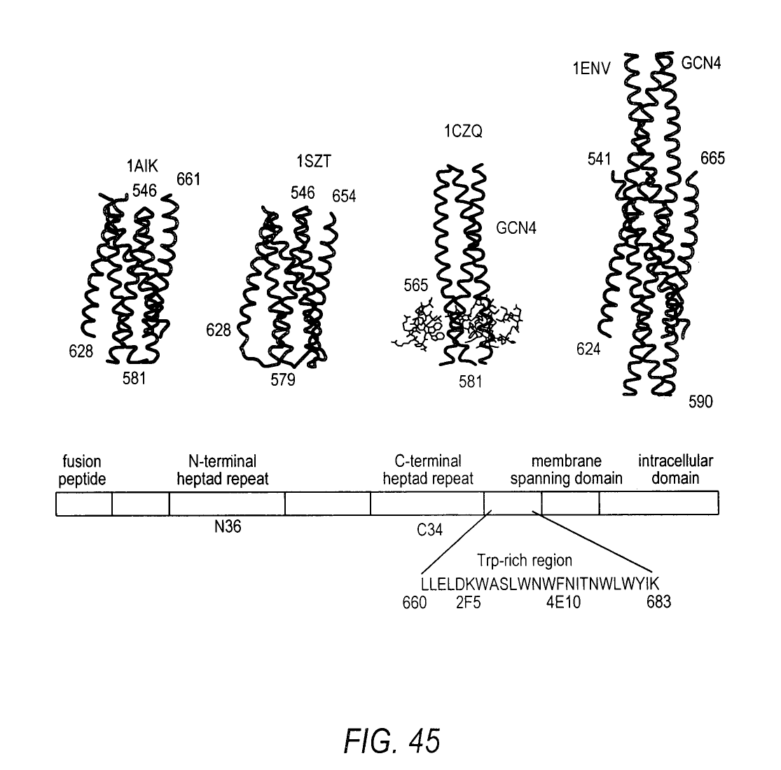

Additionally, substantial structural information is also now available for the fusion-active form of gp41, with at least eighteen different crystal structures in the PDB representing variants of the protease-resistant core of the HIV-1 gp41 ectodomain (FIG. 45) (Weissenhorn, 1997; Chan, 1997; Eckert, 1999; Tan, 1997; Ji, 1999; Shu, 2000a; Shu, 2000b; Liu, 2001; Zhou, 2000; Lu, 2001). Additionally, x-ray and NMR structures are available for the related SIV gp41 (Yang, 1999; Malashkevich, 1998; Caffrey, 1998; Kuszewski, 1999; Liu, 2002), Ebola virus GP2 cores (Malashkevich, 1999; Weissenhorn, 1998) and visna virus core (Malashkevich, 2001). The fusion-active form of gp41 is a bundle of six helices with three inner helices (N-terminal heptad repeat; NHR) forming a trimeric coiled-coil and three outer helices (C-terminal heptad repeat; CHR) packing anti-parallel to the inner trimer (FIG. 45). The first gp41 core structures were for the N36/C34 complex (FIG. 45, 1AIK, (Chan, 1997)), and a single fusion peptide with a trimeric GCN4 sequence N-terminal to gp41 residues 546-596 (NHR), followed by 628-670 (CHR) (FIG. 45, 1ENV, (Weissenhorn, 1997)). Other structures include a fusion peptide containing the NHR region (551-584) linked by residues SGGRGG (SEQ ID NO: 84) to the CHR region (633-659) (FIG. 45, 1SZT, (Tan, 1997)) in different detergents, and with mutations in several positions (Ji, 1999; Shu, 2000a; Shu, 2000b). Finally, the structure of a peptide (IQN17) designed to solubilize N36 by fusing a trimeric GCN4 sequence to a mutated NHR sequence was determined as a complex with a fusion inhibiting D-amino acid peptide (FIG. 45, 1CZQ, (Eckert, 1999)). All of these structures are presumed to represent the fusion active form of the gp41 ectodomain. Comparison with the pre-fusion (Wilson, 1981) and the fusion active forms (Bullough, 1994; Chen, 1999) of the influenza virus hemagglutinin, reveals some similarity of the HIV-1 gp41 structure and fusion mechanism to that of the influenza virus hemagglutinin HA2. These short-lived fusion intermediates expose new epitopes that may provide additional neutralization targets, or facilitate design of fusion inhibitors, such as peptides (FIG. 45) (Eckert, 1999; Wild, 1994; Jiang, 1993; Jiang, 1993; Rimsky, 1998; Ferrer, 1999) and small molecules (Jiang, 2000).

Other structural information for gp41 includes IR spectroscopy of the N-terminal fusion peptide (Gordon, 2004), an NMR structure of the Trp-rich membrane proximal region (KWASLWNWFNITNWLWYIK; SEQ ID NO: 1) bound to micelles (Schibli, 2001), and several NMR studies of the 2F5 epitope, part of the same Trp-rich region (Barbato, 2003; Biron, 2002). These studies all indicate that the fusion peptide and the membrane proximal region can adopt helical conformations, at least in apolar environments.

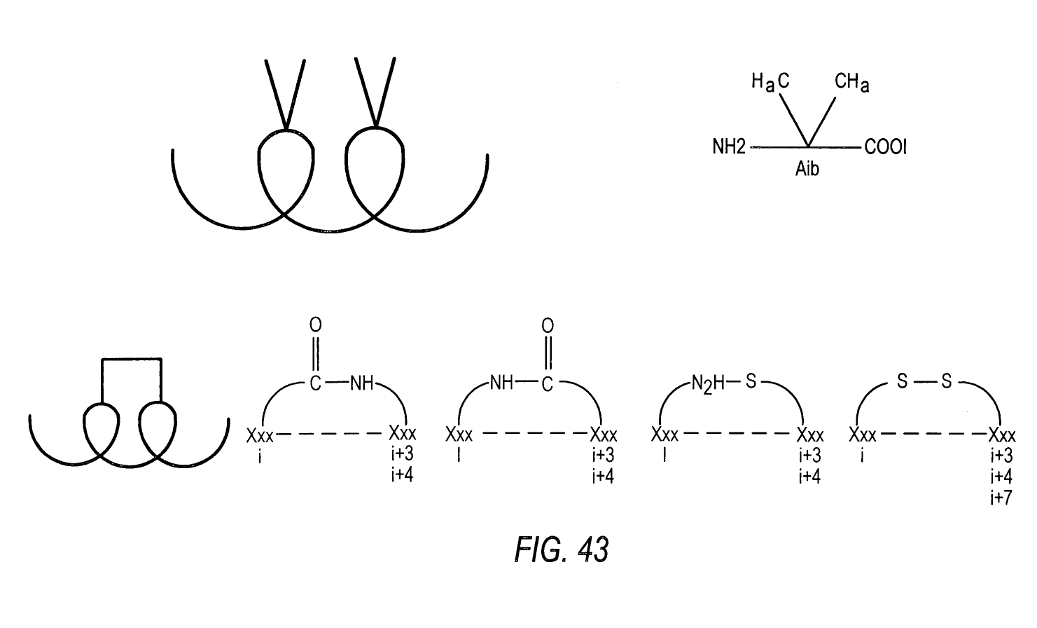

As stated, the 4E10 epitope appears to adopt a helical conformation; therefore a first generation of peptide mimics with a α-helix conformation has been designed. Among the different techniques available to increase the helicity of a peptide is the formation of constrained cyclic peptides and the introduction of the unusual amino acid amino isobutyric acid. Schematic representations of the different peptides that have or will be synthesized, as well as the structure of Aib are shown in FIG. 43. Peptides belonging to three different categories have been designed and synthesized: cycloethers, lactams, Aib-containing peptides.

Furthermore, initial results on the ability of some peptides to bind 4E10, 2F5 and Z13, have provided insight on the importance of the sequence NWFDIT (SEQ ID NO: 85), which appears to be more promising than NWFNIT (SEQ ID NO: 86) to generate broadly neutralizing antibodies. The presence of aspartic acid appears to be crucial to allow binding to 4E10.

The goal of this experiment was to synthesize peptides, or peptidomimetics, with a helical conformation and with the key amino acids. A large number of peptides have been synthesized with increasing diversity in the structures. To enhance helicity, an amino isobutyric acid (Aib) may be introduced, or a (i, i+3), a (i, i+4), or a (i, i+7)¹⁷ cyclic peptide may be formed, for example.

Compounds from three main families were designed and synthesized: the Aib-containing peptides (Aib stands for amino isobutyric acid (an unnatural amino acid that induces a local helical backbone structure)), the cyclic thioethers, and the cyclic lactams. The variety of examples from each family can be expanded by changing the sequence of the amino acids and the size of the ring.

For compounds in the Aib family, the position of the substitution(s) and the length of our peptides are being studied. In the lactan family of compounds, (i, i+4) derivatives based on the sequences c(EXXXK) (a side chain cyclized peptide between Glu and Lys to induce helicity) and c(KXXXE) (the reverse of the c(EXXXK) side chain) have been synthesized. The diversity of these compounds is expanded by replacing lysine with ornithine, which reduces the ring size. Compounds in a (i, i+3) model are also being designed. This allows a determination of which ring size seems more appropriate, and whether the amide bond should be reversed. Additionally, in the cyclothioether compounds, the size of the ring is also studied by replacing the initial c(CXXXO) sequence (a sidechain cyclized peptide with a thioether bond between Cys and a bromoacetylated ornithine residue) with c(OXXXC), c(KXXXC).

Other methods to increase the peptide helicity include introduction of an α-aminoisobutyric acid residue (AIB), or crosslinking the helix with lactam, thioether, or disulfide bridges (FIG. 43)

Additionally, circular dichroism (CD) experiments are performed on each compound to assess their helicity content.

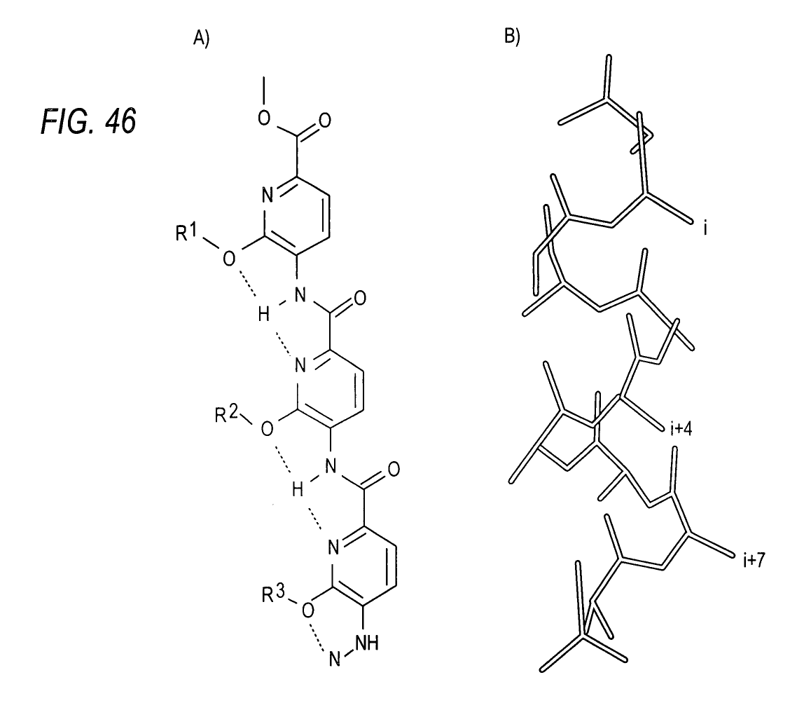

Fifty-five different peptides have already been synthesized (Table 4, the —NH2 at the C-terminus means the peptides are amides; the poly Arg or poly Lys tails are for solubility, not for 4E10 binding). Thus, small molecule α-helix mimetics that present the side chains of the Ab bound hydrophobic face of the amphipathic α-helix (residues (672-680) are prepared, examined for 4E10 Ab binding, and ultimately enlisted as antigens to elicit Mabs capable of binding the conserved gp41 core epitope. Since the Ab-antigen recognition comes from steric and chemical complementarity derived from a mostly hydrophobic Ab cavity and since the bound peptide antigens adopt an α-helix conformation with internal (versus Ab-peptide) hydrogen bonds, the recognition depends mainly on the hydrophobic side chain interactions with the hydrophobic Ab binding site. These can be synthetically reproduced by displaying the key side chains on α-helix mimetics designed to appropriately display the recognition face (side chains of Trp^{P672}, Phe^{P673}, Ile^{P675}, Thr^{P676} and Trp^{P680}) on a small molecule (e.g. i, i+3, and i+7 residues). Included in the list of peptides in Table 4 is one such mimetic that was based on a design from the Hamilton lab (Ernst, 2003; Kutzki, 2002) (FIG. 46). Furthermore, tight binding peptides for 4E10 from the Scott lab are also selected from peptide libraries displayed on the major coat protein of filamentous bacteriophage (pVIII) (Scott, 1990) and include cyclic peptide E6.8 (RCRTIDVFRNCI; SEQ ID NO: 17) and linear peptide 10A.3 (AEPAETSWFYLTTFL; SEQ ID NO:18).

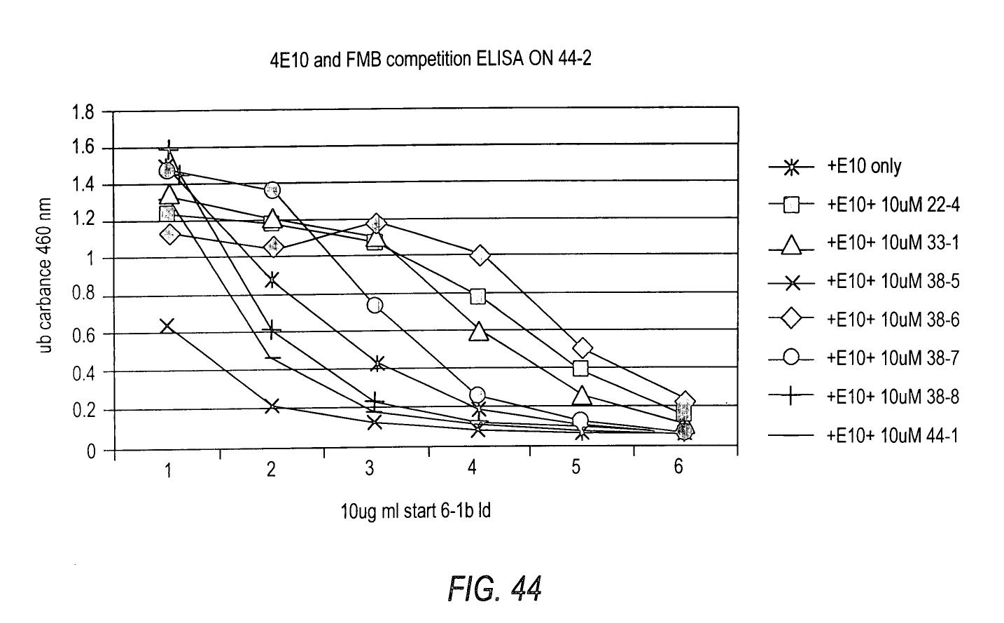

The binding of these peptides with the different epitopes has been studied by ELISAs. The affinity of peptides binding to 4E10 has been increased, as can be seen on the ELISA chart in FIG. 44. This figure depicts competition assays on 44-2 (native sequence) with different peptides: a cycloether (22-4), an Aib-containing peptide (33-1), some lactams (38) and a shorter native sequence.

As a second consideration to the design of peptides described above, it is preferred that the non 4E10 binding elements of the peptides also be engineered to be as non-immunogenic as possible. Accordingly the minimum elements required to obtain the best binding are identified and all non-crucial elements are rendered as non-immunogenic as possible to reduce the likelihood of non-neutralizing epitopes and the formation of non-neutralizing antibodies; only the key binding elements need to be present, the remainder can be replaced by alanine when possible (because alanine is poorly immunogenic) or by the least immunogenic substituents. The present compounds bind tightly to the 4E10 antibody; and, following immunization, the elicited antibodies will be tested in a single-round infectivity neutralization assay against the sensitive HIV-1 strain HxB2. Pre-immune serum will be included as a negative control. The neutralization will be confirmed using purified IgGs from the serum in the neutralization assay against HxB2 and a less neutralization-sensitive isolate, JR-FL. In parallel, the sera will be titered against the peptides in our panel to determine their breadth and specificity, in comparison with 4E10.

TABLE 4
4E10 peptides synthesized in the Dawson lab.

| | Name | SEQUENCE |
|---|---|---|
| 1 | 44-1 | NWFDITNWLWRR-NH₂ |
| | | (SEQ ID NO: 19) |
| 2 | 44-2 | SLWNWFDITNWLWRR-NH₂ |
| | | (SEQ ID NO: 20) |
| 3 | 44-3 | DKWASLWNWFDITNWLWRR-NH₂ |
| | | (SEQ ID NO: 21) |
| 4 | 84-1 | NWFDITNWLWKKKK-NH₂ |
| | | (SEQ ID NO: 15) |
| 5 | 84-2 | WNWFDITNWLWKKKK-NH₂ |
| | | (SEQ ID NO: 22) |
| 6 | 84-3 | LWNWFDITNWLWKKKK-NH₂ |
| | | (SEQ ID NO: 23) |
| 7 | 84-4 | SLWNWFDITNWLWKKKK-NH₂ |
| | | (SEQ ID NO: 24) |
| 8 | 85-1 | NWFDITNWLAKKKK-NH₂ |
| | | (SEQ ID NO: 25) |
| 9 | 85-2 | WNWFDITNWLAKKKK-NH₂ |
| | | (SEQ ID NO: 26) |
| 10 | 85-3 | LWNWFDITNWLAKKKK-NH₂ |
| | | (SEQ ID NO: 27) |
| 11 | 85-4 | SLWNWFDITNWLAKKKK-NH2 |
| | | (SEQ ID NO: 28) |
| 12 | 25-1 | Ac-WFDIT-Aib-NH₂ |
| | | (SEQ ID NO: 29) |
| 13 | 25-2 | Ac-NWFDIT-Aib-NH₂ |
| | | (SEQ ID NO: 30) |
| 14 | 29-1 | Ac-Aib-NWFDIT-Aib-NH₂ |
| | | (SEQ ID NO: 31) |
| 15 | 29-3 | Ac-DKWASL-Aib-NWFDIT-Aib-NH₂ |
| | | (SEQ ID NO: 32) |
| 16 | 29-4 | Ac-ELDKWASL-Aib-NWFDIT-Aib-NH₂ |
| | | (SEQ ID NO: 33) |
| 17 | 33-1 | NWFDITN-Aib-LWRR-NH₂ |
| | | (SEQ ID NO: 34) |
| 18 | 33-2 | SL-Aib-NWFDITN-Aib-LWRR-NH₂ |
| | | (SEQ ID NO: 35) |
| 19 | 33-3 | DKW-Aib-SL-Aib-NWFDITN-Aib-LWRR-NH₂ |
| | | (SEQ ID NO: 36) |
| 20 | 22-1 | Ac-CAWFO(Ac)IT-NH₂ |
| | | (SEQ ID NO: 37) |
| 21 | 22-2 | Ac-c(CAWFO)IT-NH₂ |
| | | (SEQ ID NO: 38) |
| 22 | 22-3 | CAWFO(Ac)IT-NH₂ |
| | | (SEQ ID NO: 39) |
| 23 | 22-4 | c(CAWFO)IT-NH₂ |
| | | (SEQ ID NO: 40) |
| 24 | 24-1 | KKCAWFO(Ac)IT |
| | | (SEQ ID NO: 41) |
| 25 | 24-2 | Ac-KKc(CAWFO)IT-NH₂ |
| | | (SEQ ID NO: 42) |
| 26 | 31-1 | c(CNWFO)ITNWLWRR-NH₂ |
| | | (SEQ ID NO: 43) |
| 27 | 31-2 | CNWFO(Ac)ITNWLWRR |
| | | (SEQ ID NO: 44) |
| 28 | 31-3 | DKWASLc(CNWFO)ITNWLWRR-NH₂ |
| | | (SEQ ID NO: 45) |
| 29 | 31-4 | DKWASLCNWFO(Ac)ITNWLWRR-NH₂ |
| | | (SEQ ID NO: 46) |
| 30 | 31-5 | LELDKWASLc(CNWFO)ITNWLWRR-NH₂ |
| | | (SEQ ID NO: 47) |
| 31 | 31-6 | LELDKWASLCNWFO(Ac)ITNWLWRR-NH₂ |
| | | (SEQ ID NO: 48) |
| 32 | 70-1 | CWFOITNWLWKK-NH₂ |
| | | (SEQ ID NO: 49) |
| 33 | 70-2 | CWFOITNWLWKK-NH₂ |
| | | (SEQ ID NO: 50) |
| 34 | 70-4 | WCWFOITNWLWKK-NH₂ |
| | | (SEQ ID NO: 51) |
| 35 | 74-1 | CWFOITNWLWKKKK-NH₂ |
| | | (SEQ ID NO: 52) |
| 36 | 74-2 | c(CWFO)ITNWLWKKKK-NH₂ |
| | | (SEQ ID NO: 53) |
| 37 | 74-3 | WCWFOITNWLWKKKK-NH₂ |
| | | (SEQ ID NO: 54) |
| 38 | 74-4 | Wc(CWFO)ITNWLWKKKK-NH₂ |
| | | (SEQ ID NO: 55) |
| 39 | 38-1 | NWFEITNKLWGRRRRC |
| | | (SEQ ID NO: 56) |
| 40 | 38-2 | NWFc(EITNK)LWGRRRRC |
| | | (SEQ ID NO: 57) |
| 41 | 38-3 | LWNWFEITNKLWGRRRRC |
| | | (SEQ ID NO: 58) |
| 42 | 38-4 | LWNWFc(EITNK)LWGRRRRC |
| | | (SEQ ID NO: 59) |
| 43 | 38-5 | DKWASLWNWFEITNKLWGRRRRC |
| | | (SEQ ID NO: 60) |
| 44 | 38-6 | DKWASLWNWFc(EITNK)LWGRRRRC |
| | | (SEQ ID NO: 61) |
| 45 | 38-7 | LLELDKWASLWNWFEITNKLWGRRRRC |
| | | (SEQ ID NO: 62) |
| 46 | 38-8 | LLELDKWASLWNWFc(EITNK)LWGRRRRC |
| | | (SEQ ID NO: 63) |
| 47 | 41-1 | NWFEITNWLWGRRRRC |
| | | (SEQ ID NO: 64) |
| 48 | 41-3 | DKWASLKNWFEITNWLWGRRRRC |
| | | (SEQ ID NO: 65) |
| 49 | 41-4 | DKWASLc(KNWFE)ITNWLWGRRRRC |
| | | (SEQ ID NO: 66) |
| 50 | 41-5 | LLELDKWASLKNWFEITNWLWGRRRRC |
| | | (SEQ ID NO: 67) |
| 51 | 41-6 | LLELDKWASLc(KNWFE)ITNWLWGRRRRC |
| | | (SEQ ID NO: 68) |
| 52 | 76-1 | EWFKITNWLWKKKK-NH₂ |
| | | (SEQ ID NO: 69) |
| 53 | 76-2 | c(EWFK)ITNWLWKKKK-NH₂ |
| | | (SEQ ID NO: 70) |
| 54 | 76-3 | WEWFKITNWLWKKKK-NH₂ |
| | | (SEQ ID NO: 71) |
| 55 | 76-4 | Wc(EWFK)ITNWLWKKKK-NH₂ |
| | | (SEQ ID NO: 72) |

Additionally, monoclonal antibodies against the 4E10 epitope will be isolated and their specificity compared with 4E10 against the panel of peptides. The monoclonal antibodies will also be tested in neutralization assays. The “WF” of the core 4E10 epitope, NWFDIT (SEQ ID NO: 85), appears to be significant for 4E10 binding and this will be confined in other antibodies to this region of gp41 in order for them to neutralize HIV-1.

Additionally, to improve the non-immunogenicity of the helical peptides, the peptides will be “masked” on the side of the helix that is not involved in the binding using, for instance, C-sugars (such as those described in U.S. patent application Ser. No. 10/471,328). Sugars are known to be poorly immunogenic because of their bulk, and C-sugars present the advantage of an increased enzymatic stability. C-sugars would be attached on the functional side chains of amino acids placed on the inert phase of the helix (Brunel, 2003a; Brunel, 2003b).

### Example 4

### Synthesis and Characterization of Peptides and Peptidomimetics

To identify the minimal gp41 peptide sequence that binds tightly to 4E10, a series of peptides were synthesized. Previous studies had identified the residues NWFDIT (SEQ ID NO: 85) (gp41 671-676) to be an important part of the core 4E10 epitope (Stiegler, G., 2001; Zwick, M. B., 2001). The importance of W680 was also shown from alanine scanning mutagenesis of the gp41 membrane proximal envelope region (MPER) on the virus using 4E10 neutralization as a readout, and also suggested from analysis of the crystal structure of a 13-amino acid peptide “KGND”, which includes gp41 residues 669 to 677 bound to 4E10 (Cardoso, R. M., 2005; Zwick, M. B., 2005). Therefore, the sequence NWFDITNWLW (SEQ ID NO: 87) corresponding to gp41 residues 671-680 was selected as a starting point to identify the full linear epitope.

Peptides were synthesized manually using solid phase peptide methodology on a C-terminal amide yielding MBHA resin, using in situ neutralization cycles for Boc-solid phase peptide synthesis (Schnolzer, M., 1992). Aib was activated using 0.5 mmol Boc-Aib-OH, 0.5 mmol TFFH and 0.7 ml DIEA in 1.5 ml DMF for 15 minutes at 25° C. The activated amino acid was added to the deprotected polypeptide resin without prior neutralization and coupled for 20 minutes. When necessary, double couplings were performed. The N-termini of the peptides were left unprotected. Solubilizing tails were introduced on the C-terminal end of the peptide to allow easier synthesis of multiple compounds. Following chain assembly, the peptides were cleaved from the resin with HF and 10% anisole for 1 hour at 0° C.

The peptides were purified by analytical reverse-phase HPLC, performed on a Rainin HPLC system equipped with a Vydac C18 column (10 mm, 1.0×15 cm, flow rate 1 mL/min). Preparative reverse-phase HPLC was performed on Waters 4000 HPLC system using Vydac C18 columns (10 μm, 5.0×25 cm) and a Gilson UV detector. Linear gradients of acetonitrile in water/0.1% TFA were used to elute bound peptides. Peptides were characterized by electrospray ionization mass spectrometry on an API-III triple quadruple mass spectrometer (Sciex, Thornhill, Ontario, Canada). Peptide masses were calculated from the experimental mass to charge (m/z) ratios from all of the observed protonation states of a peptide by using MacSpec software (Sciex). All observed peptide masses agreed with the calculated average masses within 0.5 Da.

IC₅₀, were determined by competitive ELISA using a constant concentration of biotinylated peptide and IgG with a variable concentration of gp41 peptides. Microwells were coated overnight at 4° C. with 50 μl PBS containing neutravidin (Pierce; 4 μg/ml). Wells were washed twice with PBS containing 0.05% Tween 20, and blocked with 4% non-fat dry milk in PBS for 45 minutes at 37° C. A mixture of a biotinylated 4E10-epitope peptide, SLWNWFDITNWLWRRK(biotin)-NH₂ (SEQ ID NO: 88) (20 nM), IgG 4E10 (0.2 nM), and the competing peptide analog (3-fold dilution series starting at 10 μM) in 0.4% non-fat dry milk, 0.02% Tween and PBS was incubated in a separate 96-well plate at 37° C. for 2 hours. After washing the blocked plate, the mixture of 4E10, biotinylated peptide and competing peptide was added to the wells. After 20 minutes at room temperature, the Wells were washed five times, and a 1:500 dilution of goat anti-human IgG F(ab′)₂ HRP conjugate (Pierce) was added. Following incubation at RT for 40 minutes, the wells were washed five times, and developed by adding 50 μl of TMB solution (Pierce) according to the manufacturer's instructions. After ˜20 minutes, wells containing TMB solution were stopped by adding 50 μl of H₂SO₄ (2M) and the O.D. at 450 nm was read on a microplate reader (Molecular Devices). The concentration of competitor peptide corresponding to a half-maximal signal (IC₅₀) was determined by interpolation of the resulting binding curve. Each peptide competitor was tested in duplicate in at least two separate experiments.

The resulting peptide NWFDITNWLWKKKK-NH₂ (SEQ ID NO: 15) had an IC₅₀ of 40 nM. The extent of the 4E10 peptide epitope was characterized by extending this sequence towards the N and C-termini. N-terminal extensions of the epitope did not improve 4E10 binding. C-terminal extension of the sequence up to the transmembrane domain (residue 683) increased 4E10 binding by 4-fold with respect to the starting peptide. The results herein suggest that residues 671-683 of gp41 (NWFDITNWLWYIK; SEQ ID NO: 73) represent the shortest linear epitope with optimal affinity for 4E10. A peptide encompassing this sequence with a solubilizing lysine tail, NWFDITNWLWYIKKKK-NH₂ (SEQ ID NO: 8), had an IC₅₀ of 10 nM, an improvement of 4-fold over the starting peptide and an improvement over 1000-fold compared to KGND, a 13 mer co-crystallized with 4E10. Table 5 shows the amino acid sequences and binding data to 4E10 of selected unconstrained peptide analogs.

TABLE 5
Amino acid sequences and 4E10 binding data
(IC₅₀ and K_{d}) of selected
unconstrained peptide analogs

| | | IC₅₀ | K_{d} |
|---|---|---|---|
| Peptide | Structure | (nM) | (nM) |
| 84-1 | NWFDITNWLWKKKK-NH₂ | 40 | 100 |
| | (SEQ ID NO: 15) | | |
| 84-2 | WNWFDITNWLWKKKK-NH₂ | 120 | nd |
| | (SEQ ID NO: 74) | | |
| 84-4 | SLWNWFDITNLWLKKKK-NH₂ | 120 | nd |
| | (SEQ ID NO: 15) | | |
| 104-1 | NWFCITOWLWKKKK-NH₂ | 40 | nd |
| | (SEQ ID NO: 7) | | |
| 94-1 | NWFDITNLWLYIKKKK-NH₂ | 10 | 18 |
| | (SEQ ID NO: 8) | | |
| KGND | KGWNWFDITNWGK-NH₂ | >10,000 | nd |
| | (SEQ ID NO: 2) | | |

“O” represents the unnatural amino acid ornithine. In peptide 104-1, the side chain was acylated. “nd” in Table 5 means “not determined”.

The importance of individual amino acid side chains were assessed by performing alanine-scanning mutagenesis. Alanine was individually substituted for each amino acid in the optimized epitope (residues 671-683). The effects of these mutations on the IC₅₀ are shown in FIG. 47. Mutations at W672, F673, and T676 resulted in a major decrease in binding to the 4E10 antibody (over 1,000-fold), and confirm that these three residues are crucial for peptide recognition by 4E10. The next major increase in IC₅₀ was observed when L679 was mutated to alanine. The importance of this residue had not been predicted in prior reports. Four other residues (N671, D674, 1675, and W680) also showed a decrease in binding of 20 to 30-fold when alanine substitutions were performed. The other residues in the sequence could be substituted to alanine without any major decrease in 4E10 binding (five-fold or less).

Structural analyses of the 4E10/peptide complex showed that the bound conformation of the peptide is helical (Cardoso, R. M., 2005). Therefore, helix-inducing constraints were introduced, including Aib residues and side chain tethers. Table 6 contains the peptide sequences and binding constants of the constrained peptides. Peptides in which “WF” was not included in the cyclic tether showed substantially increased binding to 4E10, indicating that these particular constraints on “WF” interfere with binding. Constraints in the center and C-terminus resulted in peptides with a tighter binding to 4E10, suggesting that increasing the helical character in these regions is favorable for 4E10 binding. The results herein are consistent with the crystal structure of “KGND” bound to the antibody in which the helix begins to “unwind” at residues W672 (Cardoso, R. M., 2005). Tightly binding peptides (IC₅₀ of 10 nM) were obtained that incorporated either Aib residues or thioether tethers.

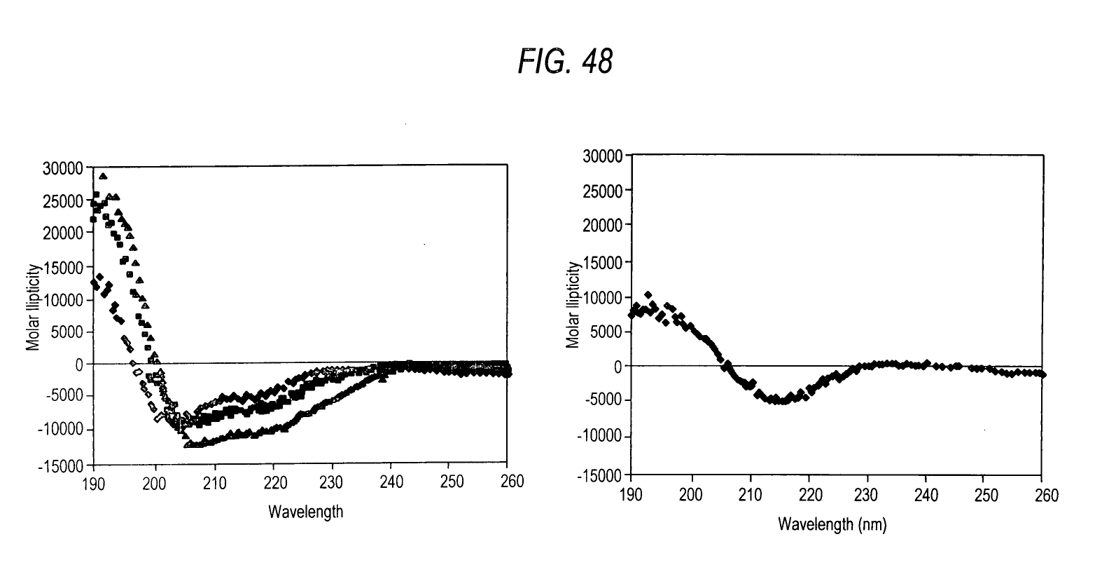

To determine whether the imposed constraints increased the helicity of the peptides, each one was analyzed in solution using circular dichroism (CD) spectroscopy (FIG. 48). An Aviv spectropolarimeter Model 203-02 was used, with cells of 0.1 cm in length, a wavelength step of 0.5 nm and a bandwidth of 1.0 nm. One to three scans were reported. The exact peptide concentrations were determined by UV measurements at 280 nm on a Gison UV detector, model 116.

The tightest binding peptides were all helical with minima close to 207 and 222 nm. However, a further increase in helicity did not result in an increase in binding: 94-1 is more helical than 84-1, but has a smaller IC₅₀. Peptide 119, which is more helical 94-1, had the same IC₅₀. Nevertheless, the imposed constraints were able to increase the peptide order in solution without diminishing 4E10 binding. Slightly shorter, structurally constrained peptides with tight binding to 4E10 (IC₅₀=10 nM) were also identified (see peptides 102-1 and 104-2).

TABLE 6
Amino acid sequences and 4E10 binding data
(IC₅₀ and K_{d}) of selected
constrained analogs.

| | | IC₅₀ | K_{d} |
|---|---|---|---|
| Peptide | Structure | (nM) | (nM) |
| KGND | KGWNWFDITNWGK-OH | >10,000 | nd |
| | (SEQ ID NO: 2) | | |
| 102-1 | NWFDITNWLWKBKBK-NH₂ | 10 | nd |
| | (SEQ ID NO: 9) | | |
| 102-2 | KKBNWFDITNWLWKBKBK-NH₂ | 10 | nd |
| | (SEQ ID NO: 10) | | |
| 119 | NWFDITNWLWYIKBKBKK-NH₂ | 10 | nd |
| | (SEQ ID NO: 75) | | |
| 74-2 | CWFOITNWLWKKKK-NH₂ | 230 | 276 |
| | (SEQ ID NO: 52) | | |
| 104-2 | NWFCITOWLWKKKK-NH₂ | 10 | 17 |
| | (SEQ ID NO: 89) | | |

“B” refers to the amino acid residue Aib (amino isobutyric acid). The underlined amino acids are in a cyclic conformation. Such a sequence containing C,O is a cyclic thioether.

The affinitiefs of the peptide analogs for 4E10 were also measured by surface plasmon resonance. Surface plasmon resonance experiments were performed using a Biacore 2000 instrument (Uppsala, Sweden). Around 2,200 response units (RU) of Fab 4E10 were coated on CM5 chips. The carboxyl groups on the chip were activated with 1-ethyl-3-(3-dimethylaminopropyl)carbodiimide hydrochloride (EDC) and N-hydroxysuccinimide (NHS). Fifty micrograms of Fab were diluted in 10 mM sodium acetate pH 4.5; a flow rate of 5 μl/min was used. Unreacted carboxyl groups were blocked with 1M ethanolamine at pH 8.5. The control was treated in the same fashion without any antibody present. Different amounts of free peptides were then passed over the surfaces at 30 or 50 μl/min for 2 min. Regeneration was done in HPS-EP buffer, 0.25 NaCl (Biacore) in 10 minutes. The amount of salt was increased compared to the commercial buffer to reduce the non-specific binding. For data evaluation, the BIAevaluation software was used. RI and Rmax were controlled, double referencing were done (0 concentration and start point). Analyses were performed to achieve the best curve fitting and small chi² (<1).

The K_{d} values obtained from the Biacore analysis were in good agreement with the ELISA results and were all within a factor of 1.5-2.5 higher than the corresponding IC₅₀ values as determined by ELISA (see Tables 5 and 6). The affinity-optimized native sequences, as well as several of the constrained peptides, all bind the 4E10 neutralizing antibody with affinities in the nanomolar range (K_{d} 20 nM). Their IC₅₀s were determined by ELISA to be around 10 nM. The recurrence of 10 nM values in the ELISA of the tightest binding peptides to 4E10 could mean that a sensitivity limit might have been reached in the assay even though lower IC₅₀ₛ could theoretically be measured. However, in the two examples chosen, peptides with an IC₅₀ of 10 nM were confirmed via Biacore to have similar K_{ds} (18 and 17 nM for 94-1 and 104-2, respectively). Note than an IC₅₀ of 0.25 μg/ml was determined for recombinant gp41 (residues 541-682 of HxB2; Viral Therapeutics, Inc., Ithaca, N.Y.), which, if it is assumed that gp41 has an average molecular weight of 25 kDa and is largely monomeric in solution, is equal to an IC₅₀ around 10 nM. However, this value can differ substantially if gp41 is not monomeric in solution.

### Example 5

### Neutralization Assays of HIV by Peptides and Peptidomimetics

To further investigate the interaction of peptide analogs and 4E10, the inhibitory effect of the best analogs on neutralization by 4E10 was assessed. Neutralization assays were performed in two different formats. In the first, replication competent HIV-1_{SF162} was assayed for neutralization using TZM-b1 cells as indicator cells (Wei, X., 2002). Alternatively, a pseudotype assay was used in which recombinant HIV-1_{JR-CSF} virions, competent for a single round of infection, were generated using the luciferase reporter plasmid pNL4-3.Luc.R-E-, as described previously (Connor, R. I., 1995; Zwick, M. B., 2003), and the pseudovirus assayed for neutralization using U87.CD4.CCR5 cells as target cells (Bjorndal, A., 1997). In all cases, the competitor peptide (NWFDITNWLWYIKKKK-NH₂; SEQ ID NO: 8) and IgG 4E10 were pre-incubated for 30 minutes at 37° C. (60 μg/ml), then the mixture was added (1:1 by volume) to HIV-1, and the resulting mixture incubated for a further hour at 37° C. The mixture of peptide, 4E10, and HIV-1 was then added (1:1 by volume) to the target cells, and the assay developed using luciferase reagent (Promega) following 48-72 hour incubation at 37° C. The degree of virus neutralization was determined as a percentage reduction of viral infectivity against an Ab-free control. All experiments were performed in triplicate and repeated at least twice with similar results.

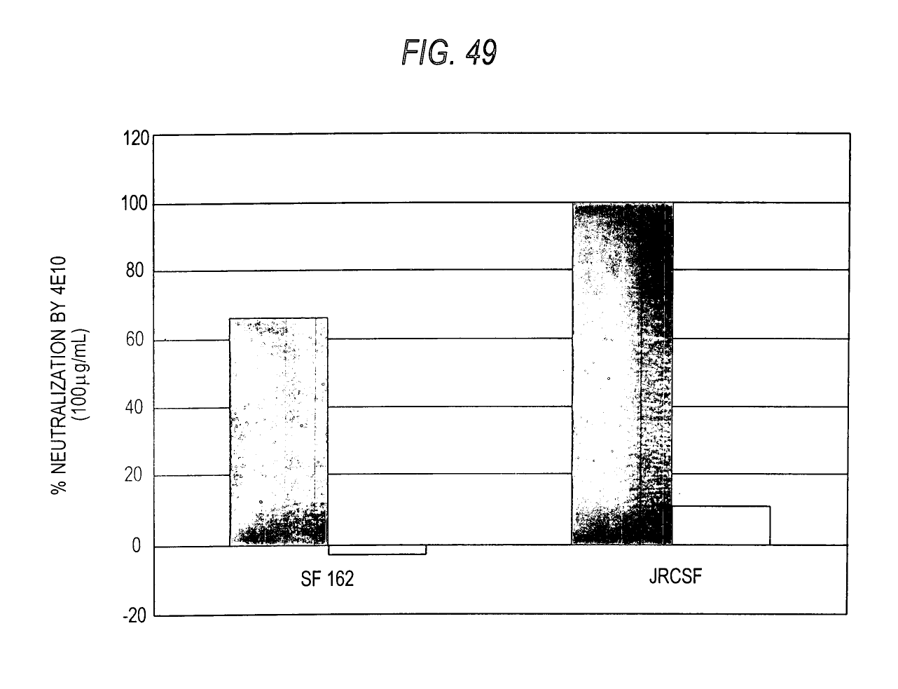

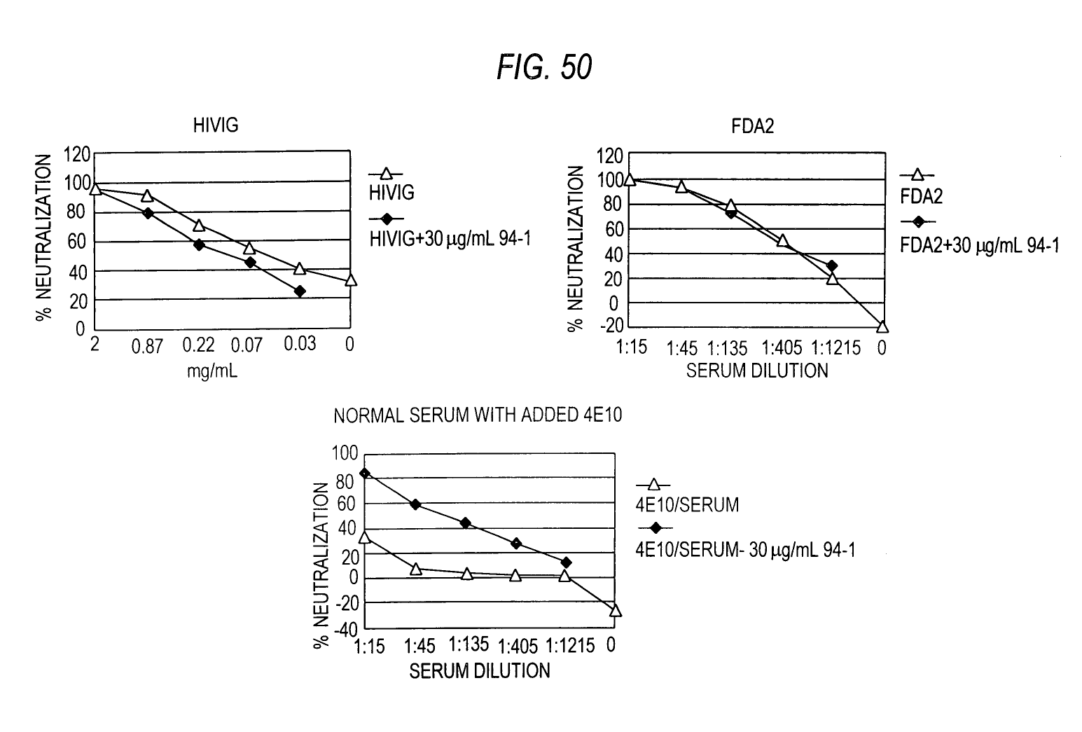

Peptide 94-1, comprising the sequence NWFDITNWLWYIKKKK-NH₂ (SEQ ID NO: 8)produced the most favorable and reproducible inhibition of 4E10 neutralization in initial experiments. This peptide could block the neutralization of 4E10 of replication competent primary isolates, SF162 and JRCSF, at 30 μg/ml (FIG. 49). The peptide also blocks neutralization under conditions, in which normal sera was spiked with 4E10 (FIG. 50). Under similar conditions, this peptide does not block neutralization by polyclonal IgG from HIV-1 infected donors, or by the reference sera, FDA2 (FIG. 50). The results herein show that the peptide interacts with the 4E10 antibody, preventing it from interacting with (and neutralizing) the virus.

### Example 6

### Structural Basis of Enhanced Binding of Long and Helically-Constrained Peptide Epitopes of the Broadly Neutralizing HIV-1 Antibody 4E10

Potent, broadly HIV-1 neutralizing antibodies (nAbs) may be invaluable for the design of an AIDS vaccine. 4E10 is the broadest HIV-1 nAB known to date and recognizes a contiguous and highly conserved helical epitope in the membrane-proximal region of gp41. The 4E10 epitope is thus an excellent target for vaccine design as it is also highly amenable to peptide engineering to enhance helical character, which should aid in eliciting 4E10-like Abs by vaccination. To investigate the structural effect of both increasing the peptide length and of introducing helix promoting constraints in the 4E10 epitope, the crystal structures of Fab $d10 bound to an optimized peptide epitope (NWFDITNWLWYIKKKK-NH₂) (SEQ ID NO: 8), an Aib-constrained peptide epitope (NWFDITNAibLWRR-NH₂) (SEQ ID NO: 34), and a thioether-linked peptide (NWFCITOWLWKKKK-NH₂) (SEQ ID NO: 89) to resolutions of 1.7 Å, 2.1 Å and 2.2 Å, respectively, have been determined. The thioether-linked peptide is the first reported structure of a cyclic tethered helical peptide bound to an antibody. The introduced helix constraints limit the conformational flexibility of the peptides without affecting interactions with 4E10. The substantial increase in affinity (10 nM versus 10⁴ nM of the IC₅₀ of the original KGND peptide template) is largely realized by 4E10 interaction with an additional helical turn at the C-terminus that includes Leu⁶⁷⁹ and Trp⁶⁸⁰, gp41 residues shown to contact CDRs H2 and H3 or 4E10. Thus, the core 4E10 epitope was extended and modified to a WFX(I/L)(T/S)XX(L/I)W motif, where X does not play a major role in 4E10 binding and can introduce constraints.

The development of a vaccine that will provide protection against exposure to HIV-1 is one of the today's most compelling medical challenges. Such a vaccine is likely to include a component that elicits broadly neutralizing antibodies against HIV-1 (Ferrantelli et al., 2002; Mascola et al., 2003; Burton et al., 2004). Some guidance as to the composition of this immunogen may be provided by the handful of broadly neutralizing human monoclonal antibodies (4E10, 2F5, 2G12 and b12) that have so far been isolated from HIV-1 infected individuals. These antibodies target conserved epitopes (Saphire et al., 2001; Calarese et al., 2003; Ofek et al., 2004; Cardoso et al., 2005) on gp120 (antibodies b12 and 2G12) or gp41 (antibodies 4E10 and 2F5), the HIV-1 envelope glycoproteins responsible for mediating viral binding and entry into human cells.

4E10 is the most broadly HIV-1 neutralizing monoclonal antibody described to date with activity against isolates from all HIV-1 clades (Binley et al., 2004). The epitopes of 4E10 and 2F5 seem particularly promising vaccines leads since these anti-gp41 antibodies are very broadly neutralizing and their epitopes are highly conserved and contiguous. However, antibodies elicted against peptides encompassing the 2F5 epitope on gp41, which have been extensively explored, are typically non-neutralizing (Coeffier et al., 2000; Joyce et al., 2002). This lack of success may be a result of the failure of the peptides to adopt a conformation similar to the native epitope in the context of the virus. Thus, restricting the peptide epitope to adopt a specific ensemble of relevant conformations will increase the probability of eliciting effective neutralizing antibody in humans. Unfortunately, the peptide epitope for 2F5 adopts a largely extended conformation (Ofek et al., 2004), and mimicking such a structure may be difficult. On the other hand, the peptide epitope for 4E10 adopts a largely helical structure (Cardoso et al., 2005), which is much more amenable to peptide engineering by introducing structural constraints.

To engineer a synthetic immunogen capable of eliciting 4E10-like antibodies, a multi-step strategy was initiated. The first step was the characterization of the epitope and its essential features in atomic detail. Antibody 4E10 recognizes a contiguous epitope in the membrane-proximal, Trp-rich region of gp41 (Zwick et al., 2004) that seems to be critical for HIV-1 entry into human cells (Salzwedel et al., 1999; Munoz-Barroso et al., 1999). The three-dimensional structural of Fab 4E10, bound to a partial peptide epitope (named KGND; KGWNWFDITNWGK-NH₂) (SEQ ID NO: 2) encompassing gp41 residues 670-678, revealed the epitope conformation and the atomic details of the antibody-epitope interaction (Cardoso et al., 2005). The bound peptide epitope adopts a helical conformation in which the key contact residues, Trp^{P672}, Phe^{P673}, Ile^{P675}, and Thr^{P676}, map to one face of the helix that is buried in an extremely hydrophobic antibody combining-site. The importance of additional flanking residues, especially at the C-terminus, has been proposed by mutagenesis studies (Zwick et al., 2005), structural modeling (Cardoso et al., 2005), and extensive analysis of various truncated peptides that encompass the 4E10 epitope (Brunel et al., 2006). The next step of the strategy focused on limiting the conformational diversity of the peptides by designing analogs that are constrained to adopt a helical conformation in solution similar to that of the peptide KGND bound to 4E10 (Brunel et al., 2006). Chemically constrained peptides have been designed to mimic helices involved in protein-protein interactions. For example, BH3 derived tethered helices directed at BCL-2 have been shown to be anti-apoptotic (Walensky et al., 2004) and nuclear eceptor co-activator helices have been shown to be potent estrogen antagonists (Leduc et al., 2003). Peptides derived from the native gp41 sequence are generally helical in PBS buffer and the presence of a helical conformation is generally associated with strong 4E10 binding (Brunel et al., 2006). To enhance helicity and reduce alternative peptide conformations, constraints were introduced to promote helical propensity through use of α-amino isobutyric acid (Aib), or through cross-linking side chains along one face of the helix with an i→i+3 thioether tether (Brunet et al., 2005).

A critical element of gp41 immunogen design is to develop conformational constrained ligands that do not introduce binding interactions that are not present in the native gp41 target. As a result, crystallographic characterization of the constrained gp41 peptides to 4E10 is critical to guide the design of second-generation 4E10 immunogens. Although there are several examples of constrained peptides that have been structurally characterized in solution by NMR and CD, there have been few studies characterizing how these constrained helices bind their protein targets (Leduc et al., 2003). To investigate the structural effect of increasing the peptide length and helix-promoting constraints in the antibody-peptide interaction, the crystal structures of Fab 4E1-in complex with a longer (compared to peptide KGND) non-constrained peptide epitope (94-1; NWFDITNWLWYIKKKK-NH₂) (SEQ ID NO: 8), an Aib-containing peptide (33-1; NWFDITN-Aib-LWRR-NH₂) (SEQ ID NO: 34), and a thioether-linked peptide epitope (104-2; NWFc(CITO)WLWKKKK-NH₂ (SEQ ID NO: 89), where c(CITO) indicates the presence of a covalent bridge linking the side chains of cysteine and ornithine) were determined. The structure of the peptide 104-2 complex is the first known example of a cyclic tethered helical peptide bound to an antibody. Peptide 33-1 is the first reported structure of a helical Aib-containing peptide bound to an antibody. Structural analysis of the 94-1, 33-1 and 104-2 complexes allowed the extension and modification of the consensus motif required for 4E10 recognition and binding.

### Peptide Synthesis and Purification

The peptides were synthesized manually using solid phase peptide methodooyg on an C-terminal amide yielding MBHA resin, using in-situ neutralization cycles for Boc-solid phase peptide synthesis (Schnolzer et al., 1992). Aib was activated using 0.15 mmol Boc-Aib-OH, 0.5 mmol TFFH and 0.7 mL DIEA in 1.5 mL DMF for 15 min, 25° C. The activated amino acid was added to the deprotected polypeptide resin without prior neutralization and coupled for 20 min. When necessary, double couplines were performed. Following chain assembly, the peptides 94-1 and 33-1 were cleaved from the resin with HF and 10% anisole for 1 h at 0° C. For peptide 104-2, following chain assembly, the Orn(Fmoc) residue was deprotected with piperidine and then bromoacetylated with bromoacetic anhydride. After side-chain deprotection and cleavage from the resin with HF, the peptide was precipitated and washed with ether. The thioether link was formed by adding 6M guanidine HCl 100 nM NaH₂PO₄, pH 8.4 to the mixture of precipitated peptide and resin (<1 mg/mL) which was stirred at RT for 2 hours.

The peptides were purified by HPLC. Analytical reserved-phase HPLC was performed on a Rainin HPLC system equipped with a Vydac C 18 column (10 μm, 1.0×15 cm, flow rate 1 mL/min). Preparative reversed-phase HPLC was performed on Waters 4000 HPLC system using Vydac C-18 columns (10 μm, 5.0×25 cm) and a Gilson UV detector. Linear gradients of acetonitrile in water/0.1% TFA were used to elute bound peptides. Peptides were characterized by electrospray ionization MS on an API-III triple quadruple mass spectrometer (Sciex, Thornhill, ON, CA). Peptide masses were calculated from the experimental mass to charge (m/z) ratios from all of the observed protonation states of a peptide by using MacSpec software (Sciex). All observed peptide masses agreed with the calculated average masses within 0.5 Da.

### Preparation of Complexes, Crystallization and Data Collection

Antigen-binding fragment Fab 4E10 was obtained by papain digestion of the recombinant IgG1(κ) 4E10 as previous described (Cardoso et al., 2005). Peptides 94-1, 33-1, and 104-2 were dissolved in dimethyl sulfoxide (DMSO) to a concentration of 50 mg/ml. Crystals of Fab4E10 in complex with the peptide were obtained by co-crystallization after overnight incubation at 4° C. of peptide and Fab4310 in a molar ratio of 1:5 (protein:peptide). The best crystals of the complexes were grown at 22° C. by sitting drop vapor diffusion against 20% (w/v) PEG 3,350 in 0.2 M ammonium nitrate in the case of the 4E10:104-2 complex, 26% (w/v) PEG 8,000 in 0.2 M sodium acetate pH 5.6 and 0.2 M sodium thiocyanate for the rE10:94-1 complex, and 36% (w/v) MPEG, 5,000 in 0.1 M sodium acetate pH 5.5 for the 4E10:33-1 complex. Prior to being cooled to cryogenic temperatures, the crystals were soaked in a cryoprotectant solution of mother liquor containing 25% (v/v) glycerol. Data were collected on beamlines 11-1 (complex with peptide 94-1) and 9-2 (complex with peptide 104-2) at the Stanford Synchotron Radiation Laboratory (SSRL), and beamline 8.2.1 (complex with peptide 33-1).at the Advanced Light Source (ALS), using a liquid nitrogen cryostream maintained at 90 K. The data sets were processed using the HKL package (Otwinowski and Minor, 1997) and the CCP4 suite of programs (Collaborative Computational Project Number 4, 1994).

### Structure Determination and Refinement

The structure of Fab 4E10 as a complex with each peptide was determined by molecular replacement using AMoRe (Collaborative Computational Project Number 4, 1994) and Fab 4E10 (PDB entry 1TZG), without the bound peptide, as a probe. A non-crystallographic translation vector for the 4E10:33-1 complex was calculated from native Patterson maps using CCP4 (Collaborative Computational Project Number 4, 1994). The structures were refined in CNS (Brunger et al., 1998). R_{free} was calculated using a set of 5% randomly assigned reflections. Fab heavy and light chains were treated separately as rigid bodies for the initial refinement. The protein model was then refined using torsion angle simulated annealing at 5,000 K. Following these initial stages, the refinement proceeding through cycles of positional, temperature factor, and manual rebuilding in XFIT (McRee, 1999) into σ_{A}-weighted 2Fₒ-F_{c} and Fₒ-F_{c} electron density omit maps. The maximum likelihood target function, bulk solvent corrections and anisotropic temperature factor corrections were used for the refinement cycles in CNS. Density for each peptide was clearly interpretable after a few cycles of refinement and manual rebuilding of the starting Fab model. Tight non-crystallographic restraints were used early on in the refinement and released gradually toward the end of the refinement. Water molecules were added manually in XFIT. Stereochemical analysis of the refined structure was performed using PROCHECK (Collaborative Computational Project Number 4, 1994). Refinement statistics are summarized in Table 8.

### Structural Analysis

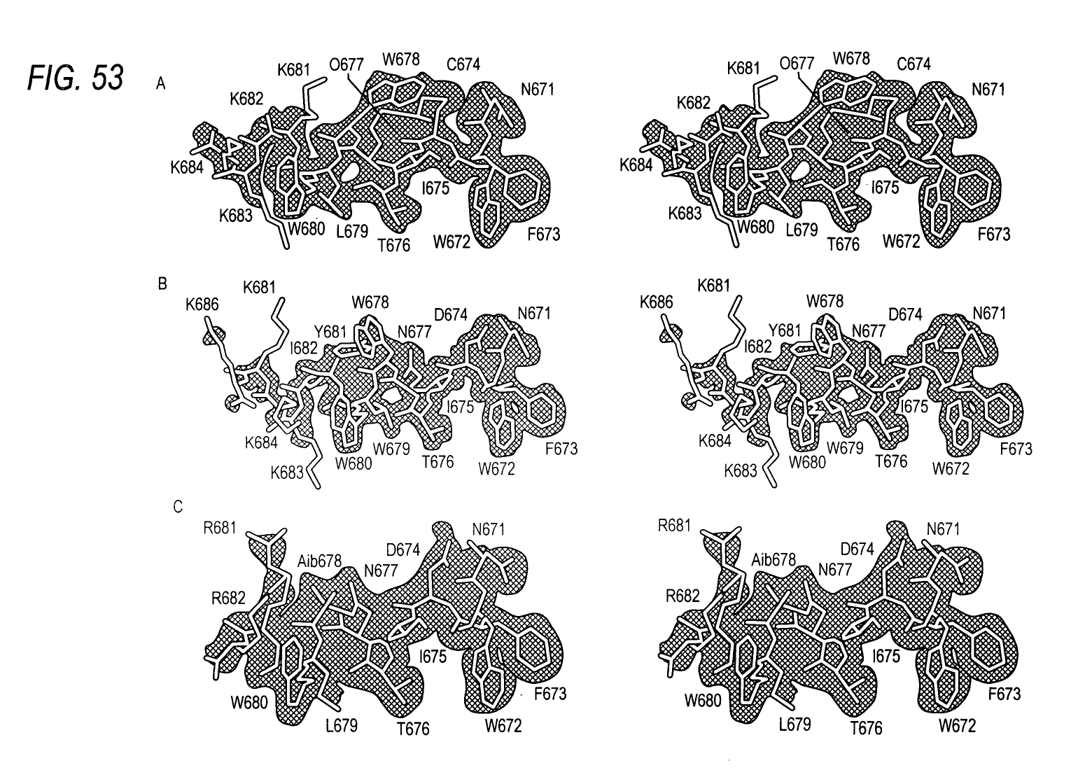

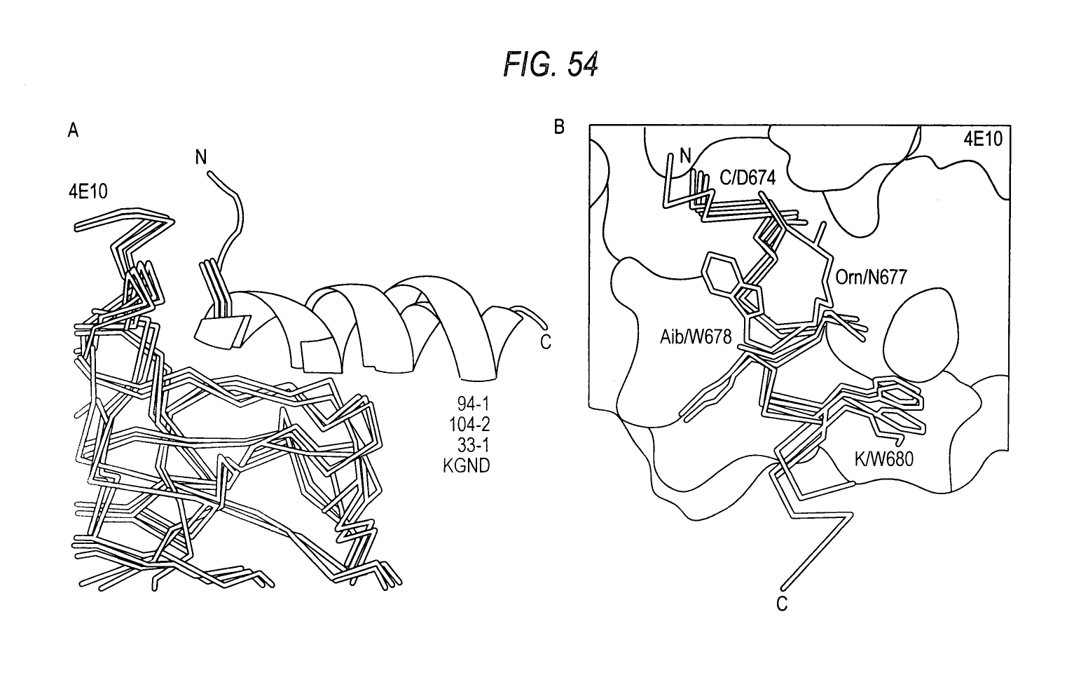

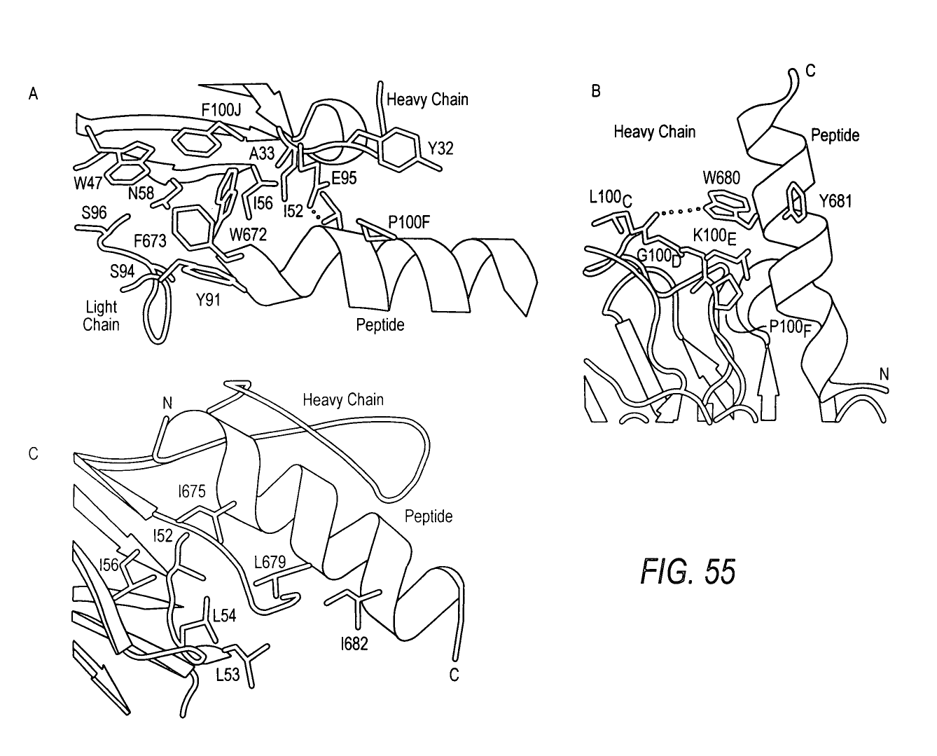

Superpositions and root mean square deviations (r.m.s.d.) calculations were carried out suing the INSIGHT II package (Accelrys, Inc., San Diego, Calif.) for pairs of C_{H}1, C_{L}, V_{H}, and V_{L} domains. Hydrogen bonds between Fab 4E10 and peptide were identified using HBPLUS (McDonald and Thornton, 1994) and van der Waals' contacts were assigned with CONTACTSYM (Sheriff et al., 1987). Buried surface areas were calculated using MS (Connolly, 1993) with a 1.7 Å probe radius and standard van der Waals radii. Secondary structure was assigned using PROMOTIF (Hutchinson and Thornton, 1996). Graphics were prepared using XFIT (McRee, 1999) (FIG. 53), RASTER3D (Merrit and Bacon, 1997) (FIG. 54), and PYMOL (DeLano, 2002) (FIGS. 54 and 55).

### Binding Affinity by ELISA

IC50s were determined by competitive ELISA using a constant concentration of biotinylated peptide and IgG with a variable concentration of gp41 peptides. Microwells were coated overnight at 4° C. with 50 μl PBS containing neutravidin (Pierce; 4 μg/ml). Wells were washed twice with PBS containing 0.05% Tween 20, and blocked with 4% non-fat dry milk (NFDM) in PBS for 45 min at 37° C. Meanwhile, a mixture of a biotinylated 4E10-peptide epitope, SLWNWFDITNWLWRRK(biotin)-NH₂ (SEQ ID NO: 88), (20 nM), IgG 4E10 (0.2 nM), and the competing peptide analogue (3-fold dilution series starting at 10 μM) in 0.4% NFDM, 0.02% Tween and PBS was incubated in a separate 96-well plate at 37° C. for 2 h. After washing the blocked plate, the mixture of 4E10, biotinylated peptide and competing peptide was added to the wells. After 20 min at room temperature, the wells were washed five times, and a 1:500 dilution of goat anti-human IgG F(ab′)₂ HRP conjugate (Pierce) was added. Following incubation at RT for 40 min, the wells were washed five times, and developed by adding 50 μl of H₂SO₄ (2 M), and the O.D. at 450 nm was read on a microplate reader (Molecular Devices). The concentration of competitor peptide corresponding to a half-maximal signal (IC50) was determined by interpolation of the resulting binding curve. Each peptide competitor was tested in duplicate in at least two separate experiments.

### Binding Affinity by Surface Plasmon Resonance

Surface plasmon resonance experiments were performed using a Biacore 2000 instrument (Uppsala, Sweden).

Chip preparation: around 2,200 response units (RU) of Fab 4E10 were coated on CM5 chips. The carboxyl groups on the chip were activated with 1-ethyl-3-(3-dimethylaminopropyl) carbodiimide hydrochloride (EDC) and N-hydroxysuccinimide (NHS). Fifty micrograms of Fab was diluted in 10 mM sodium acetate pH 4.5; a flow rate of 5 μl/min was used. Unreacted carboxyl groups were blocked with 1M ethanolamine at pH 8.5. The control was treated in the same fashion without any antibody present.

Experiment: different amounts of free peptides were then passed over the surfaces at 30 or 50 μl/min for 2 min. Regeneration was done in HPS-EP buffer, 0.25 NaCl (Biacore) in 10 min. The amount of salt was increased compared to the commercial buffer to reduce the non-specific binding.

Data evaluation: the BIAevaluation software was used. RI and Rmax were controlled, double referencing were done (0 concentration and start point). Analyses were performed to achieve the best curve fitting and small chi² (<1).

### Structure Determination and Refinement

Crystal structures of Fab 4E10 in complex with a non-constrained peptide, an Aib-containing peptide, and a thioether-linked peptide (94-1, 33-1, and 104-2 respectively, Table 7) have been determined to resolutions of 1.76 Å (94-1), 2.1 Å (104-2), and 2.2 Å (33-1). Binding of Fab 4E10 and peptide was achieved by overnight incubation of 4° C. of the Fab with a 5-fold molar excess of peptide. Crystals of each complex grew after about one week. The structures were determined by molecular replacement using the previously determined Fab 4E10 structure (PDB entry 1TZG, Cardoso et al., 2005) as the initial model, and were then re-built and refined. Data collection and refinement statistics are summarized in Table 8.

```
TABLE 7
  Amino acid sequence and 4E10 binding affinity for 4E10 peptide epitopes.
Peptide 104-2 has a covalent tether linking the side chains of cysteine
and ornithine (O)a. Peptide 33-1 has an Aib (B)b, which replaces a Trp. Peptides
44-1 and 104-1 are the controls for peptides 33-1 and 104-2, respectively. Key
  residues for 4E10 binding are residues WF, IT and LW, helix-constraining residues
are B, C and O and the tether linked region is underlined. Only IC50 values
were measured for peptides KGND, 44-1, and 104-1 (SEQ ID NOS 8, 2, 90,
19, 89 and 12 are disclosed respectively in order of appearance).
```

| Peptide | K_{d} (nM) | kₒₙ (M⁻¹s⁻¹) | k_{off} (s⁻¹) | IC50 (nM) | Sequence |
|---|---|---|---|---|---|
| | | | | | 671 680 |
| 94-1 | 20 | 6.91 × 10⁵ | 13.8 × 10⁻³ | 10 | NWFDITNWLWYIKKKK-NH₂ |
| KGND | — | — | — | >10,000 | KGWNWFDITNWGK |
| 33-1 | 302 | 4.31 × 10⁵ | 130 × 10⁻³ | 100-400 | NWFDITNBLWRR-NH₂ |
| 44-1 | — | — | — | 600 | NWFDITNWLWRR-NH₂ |
| 104-2 | 17 | 2.02 × 10⁵ | 3.4 × 10⁻³ | 10 | NWFCITOWLWKKKK-NH₂ |
| 104-1 | — | — | — | 40 | NWFCITOWLWKKKK-NH₂ |

^{a}Tether in peptide 104-2 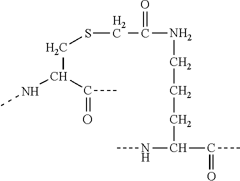 ^{b}Aib residue

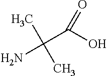

The final models contain Fab residues L1-L213, H1-H227 and peptide residues P671-P680 (33-1 and 104-2) or P671-P683 (94-1). Fab residues are numbered according to standard convention (Kabat 35 al., 1991) with light and heavy chain identifiers L and H, respectively. The peptides are numbered according to the HXB2 isolate sequence with a P chain identifier. The C-terminal residue Lys^{H228} of the heavy chain is visible only in the 94-1 complex. Electron density omit maps clearly define the location and conformation of the peptides in the 4E10 binding site (FIG. 53). The only peptide residues which have no interpretable electron density are at the C-terminus of peptide 94-1 and correspond to Lys^{P684}-Lys^{P686}, which are part of the peptide solubility tag.

The Fab-4E10-peptide structures have good geometry with only Ala^{L51}, which is in a highly conserved γ turn as in most antibody structures (Stanfield et al., 1999), in the so-called “disallowed” region of the Ramachandran plot (Table 8). As expected, the multiple complexes found in the asymmetric unit of 104-2 or 33-1 crystals are similar, with root mean square deviations (rmsd) less than 0.6 Å for C_{α} superpositions. Only the complex with the lowest B value (molecule 1) is described here.

TABLE 8
X-ray diffraction data and refinement statistics for the Fab 4E10 complexes.

| Complex Fab:peptide | | |
|---|---|---|
| 4E10:94-1 | 4E10:104-2 | 4E10:33-1 |

Crystal

| | | | |
|---|---|---|---|
| Space group | C2 | P2₁ | P2₁ |
| No. of Complexes per AU | 1 | 2 | 4 |
| Unit cell (Å, °) | a = 157.9, b = 44.6, | a = 53.4, b = 111.5, | a = 53.4, b = 113.2, |
| | c = 85.3, β = 113.1 | c = 79.4, β = 106.4 | c = 150.0, β = 94.2 |

Data Quality

| | | | |
|---|---|---|---|
| Resolution (Å)^{a} | 50.00-1.76 (1.82-1.76) | 50.00-2.10 (2.15-2.10) | 50.00-2.20 (2.28-2.20) |
| No. of observations | 197,621 | 191,855 | 221,385 |
| No. of unique reflections | 53,993 | 51,614 | 86,354 |
| Mosaicity (°) | 0.6 | 0.9 | 0.9 |
| Completeness (%)^{a} | 99.3 (99.9) | 99.3 (99.0) | 95.5 (88.9) |
| Multiplicity^{a} | 3.7 (3.7) | 3.7 (3.7) | 2.6 (1.9) |
| I/σ(I)^{a} | 14.8 (5.2) | 14.1 (2.7) | 7.3 (1.4) |
| R_{sym} (%)^{a,b} | 6.5 (30.2) | 9.4 (32.4) | 15.5 (65.4) |

Model Quality

| | | | |
|---|---|---|---|
| R_{cryst} (%)^{c} | 20.2 | 23.6 | 23.5 |
| R_{free} (%)^{c} | 22.2 | 27.1 | 28.3 |
| No. of protein atoms | 3,486 | 6,916 | 13,740 |
| No. of waters molecules | 337 | 403 | 600 |
| Average B value (Å²) | | | |
| Molecule 1 (H, L, P Chain) | 24.5, 22.0, 36.7 | 32.4, 34.1, 35.4 | 30.1, 29.3, 34.8 |
| Molecule 2 (H, L, P Chain) | na | 35.8, 34.9, 43.1 | 32.9, 34.9, 38.8 |
| Molecule 3 (H, L, P Chain) | na | na | 30.1, 29.5, 34.9 |
| Molecule 4 (H, L, P Chain) | na | na | 32.6, 36.6, 37.4 |
| Water molecules | 32.3 | 38.5 | 35.2 |
| Bond length r.m.s.d. (Å) | 0.005 | 0.006 | 0.006 |
| Bond angle r.m.s.d. (°) | 1.3 | 1.4 | 1.4 |
| Ramachandran plot regions: | | | |
| Most favored (%) | 89.9 | 87.1 | 87.5 |
| Additional allowed (%) | 9.8 | 12.4 | 12.0 |
| Generously allowed (%) | 0.0 | 0.4 | 0.3 |
| Disallowed (%)^{d} | 0.3 | 0.1 | 0.2 |

^{a}Values in parentheses correspond to the highest resolution shell.
^{b}R_{sym} = [ΣₕΣᵢ|Iᵢ(h) − <I(h)>|/ΣₕΣᵢIᵢ(h)] × 100, where <I(h)> is the mean of the I(h) observation of reflection i.
^{c}R = Σₕₖₗ|Fₒ − F_{c}|/Σₕₖₗ|Fₒ|. R_{free} was calculated as R, but using only 5% of the data reserved for the cross-validation.
^{d}The only residue in the disallowed region is Ala^{L51}, which is in a conserved γ turn, as observed in most antibody structures (Stanfield et al., 1999).

### Structure of Fab 4E10

The binding of the full linear peptide epitope and of constrained peptides encompassing the 4E10 epitope (peptides 94-1, 104-2, and 33-1) does not affect the overall conformation of the antibody and its combining site. Fab 4E10 adopts a very similar conformation in all studied complexes, as shown by the small rmsd (0.1 Å to 0.5 Å) for C_{α} superposition of pairs C_{H}1, C_{L}, V_{H}, and V_{L} domains. The Fab has the canonical β-sandwich immunoglobulin fold with an average elbow angle of 168° (±3°) for the complexes with peptides 94-1, 104-2, and 33-1.

The hydrophobic nature of the complementarity determining regions (CDR's) H2 (GVIPLLTITNYA) (SEQ ID NO: 91) and H3 (EGTTGWGWLGKPIGAFAH) (SEQ ID NO: 92) makes the 4E10 combining site considerably more hydrophobic than that of most antibodies, which facilitates 4E10 binding to its Trp-rich epitope on the membrane-proximal region of gp41. The hydrophobic tip of the CDR H3 (Gly^{H99}, Trp^{H100}, Gly^{H100A}, Trp^{H100B}, Leu^{H100C}, Gly^{H100D}) forms a surface resembling the “H3 foot” described for 2F5 (Ofek et al., 2004), another HIV neutralizing antibody that binds to a neighboring epitope within the membrane-proximal region of gp41. As only two of these H3 loop residues (Leu^{H100C} and Gly^{H100D}; Tables 9 and 10) contact the peptide epitope, the other hydrophobic residues are positioned such that they could interact with the adjacent viral membrane and/or other residues of gp41. Furthermore, the length and glycine-tryptophan-rich composition of the CDR H3 of 4E10 could be an important feature to facilitate interaction with the membrane-proximal epitope. Five Gly and two Trp residues are found within the 18 residues of the H3 loop. The Gly residues (Gly^{H96}, Gly^{H100A}, Gly^{H100D}, and Gly^{H100H}) may give the H3 loop sufficient conformational flexibility to access the membrane surface when bound to the gp41 epitope. High B values for residues at the tip of CDR H3 in all three complexes (94-1, 104-2, and 33-1) attests to the conformational flexibility of this H3 loop. The two Trp residues, located at the hydrophobic tip of the H3 loop (Trp^{H100} and Trp^{H100B}), could enhance the interaction between 4E10 and HIV by interaction of their side chains with the viral membrane when its base encounters the gp41 epitope.

TABLE 9
Van der Waals contacts between Fab4E10 and bound peptides.

| Peptide | 94-1 Complex | 104-2 Complex | 33-1 Complex |
|---|---|---|---|
| Asn^{P671} | Tyr^{L91}, Gly^{L92}, Gln^{L93}, | Lys^{L32}, Gly^{L92}, Gln^{L93}, | Tyr^{L91}, Gly^{L92}, Gln^{L93}, |
| | Ser^{L94} | Ser^{L94} | Ser^{L94} |
| Trp^{P672} | Ser^{L94}, Ala^{H33}, Gly^{H50}, | Ser^{L94}, Ala^{H33}, Ser^{H35}, | Ser^{L94}, Ala^{H33}, Gly^{H50}, |
| | Val^{H51}, Ile^{H52}, Ile^{H56}, Asn^{H58} | Gly^{H50}, Val^{H51}, Ile^{H52}, | Val^{H51}, Ile^{H52}, Ile^{H56}, Thr^{H57}, |
| | | Ile^{H56}, Asn^{H58} | Asn^{H58} |
| Phe^{P673} | Tyr^{L91}, Gln^{L93}, Ser^{L94}, | Tyr^{L91}, Gln^{L93}, Ser^{L94}, | Tyr^{L91}, Gln^{L93}, Ser^{L94}, |
| | Ser^{L96}, Trp^{H47}, Phe^{H100J} | Ser^{L96}, Trp^{H47}, Phe^{H100J} | Ser^{L96}, Trp^{H47}, Phe^{H100J} |
| ^{a}Asp^{P674}/Cys^{P674} | Lys^{L32} | Lys^{L32} | Lys^{L32} |
| Ile^{P675} | Ile^{H52}, Leu^{H54}, Ile^{H56} | Ile^{H52}, Leu^{H54}, Ile^{H56} | Ile^{H52}, Leu^{H544}, Ile^{H56} |
| Thr^{P676} | Thr^{H31}, Tyr^{H32}, Ala^{H33}, | Thr^{H31}, Tyr^{H32}, Ala^{H33}, | Thr^{H31}, Tyr^{H32}, Ala^{H33}, |
| | Ile^{H52}, Glu^{H95}, Pro^{H100F} | Ile^{H52}, Glu^{H95}, Pro^{H100F} | Ile^{H52}, Glu^{H95}, Pro^{H100F} |
| ^{a}Asn^{P677}/Orn^{P677} | Lys^{H100E}, Pro^{H100F} | Lys^{L32}, Pro^{H100F} | Lys^{H100E}, Pro^{H100F} |
| Leu^{P679} | Ile^{H52}, Leu^{H53}, Leu^{H54} | Thr^{H31}, Leu^{H53}, Leu^{H54} | Thr^{H31}, Leu^{H53}, Leu^{H54} |
| Trp^{P680} | Gly^{H100D}, Lys^{H100E}, Pro^{H100F} | Gly^{H100D}, Lys^{H100E}, Pro^{H100F} | Gly^{H100D}, Lys^{H100E}, Pro^{H100F} |
| Tyr^{P681} | Lys^{H100E} | n.a. | n.a. |

^{a}Residues P674 and P677 are cysteine and ornithine, respectively, in peptide 104-2.

TABLE 10
Hydrogen bonds and salt bridge interactions (Å) in Fab 4E10:peptide
complexes.

| | | | | 33-1 |
|---|---|---|---|---|
| Peptide atom | Fab atom | 94-1 Complex | 104-2 Complex | Complex |
| Asn^{P671}-N_{δ2} | Tyr^{L91}-O | 2.9 | 3.0 | 2.8 |
| Trp^{P672}-N | Ser^{L94}-O_{γ} | 3.1 | 3.0 | 3.1 |
| Trp^{P672}-N_{ε1} | Ile^{H56}-O | 3.4 | 3.2 | 3.2 |
| Thr^{P676}-O_{γ1} | Glu^{H95}-O_{ε1} | 3.3 | 3.2 | 3.2 |
| Thr^{P676}-O_{γ1} | Glu^{H95}-O_{ε2} | 2.7 | 2.6 | 2.6 |
| ^{a}Orn^{P677}-O_{γ1} | Lys^{L32}-N_{ξ} | n.a. | 2.5 | n.a. |
| Trp^{P680}-N_{ξ} | Leu^{H100C}-O | 3.6 | 3.2 | 3.0 |

^{a}Residue P677 is an ornithine only in peptide 104-2.

### Structure of the Peptide Epitope

Peptides 94-1, 104-2, and 33-1 include more residues of the gp41 C-terminal region than the previously studied peptide KGND (Cardoso et al., 2005). At least one additional helical turn and residues Leu^{P679} and Trp^{P680} are included in all three new peptides (Table 7). Peptide 94-1 also encompasses Tyr⁶⁸¹, Ile⁶⁸², and Lys⁶⁸³, the gp41 residues believed to be immediately adjacent to the viral membrane. The extension of the 4E10 epitope included in the new peptides was based on mutagenesis results (Zwick et al., 2005), the 4E10 crystal structure with the KGND peptide (Cardoso et al., 2005), and binding studies of variable length peptides (Brunet al., 2006) that suggested the importance of these additional gp41 residues for 4E10 binding. The final four Lys (peptides 104-2 and 94-1) or two Arg (peptide 33-1) residues at the C-terminus of each peptide were included to increase peptide solubility in water.

The helical conformation of the epitope is critical for 4E10 binding (Cardoso et al., 2005; Brunel et al., 2006). Peptides 104-2 and 33-1 were constrained to adopt an α-helical conformation using a thioether bridge and an Aib residue, respectively (Table 7 and FIG. 53). To achieve the thioether bridge in peptide 104-2, Asp^{P674} was mutated to a Cys and Asn^{P677} was changed to the unnatural amino acid Ornithine (FIG. 53B) (Brunel et al., 2006). The peptide 104-2:4 E10 complex is the first reported structure of the helical tethered side chain bound to an antibody. The Aib residue replaces Trp^{P678} in peptide 33-1 (FIG. 53C). The introduction of these constraints resulted in peptides with very similar conformation (rmsd below 0.2 Å for C_{α} superposition of peptide pairs). This confirms the efficiency of the thioether bridge to enhance the helical character of a peptide (Brunel et al., 2006). The three peptides (94-1, 104-2, and 33-1) bind to 4E10 in virtually identical orientations (FIG. 54), with each peptide in an α-helical conformation from Ile^{P675} to Lys^{P683}, preceded by a short 310 helix from Trp^{P672} to Asp^{P674} (Cys^{P674} in peptide 104-2). AsnP671, the N-terminal residue, is in an extended conformation. The membrane-proximal gp41 residues Tyr⁶⁸¹, Ile⁶⁸², and Lys⁶⁸³ are included only in the 94-1 peptide.

Peptide 104-2 superimposes onto the other peptides with a slightly different helical axis due to a different positioning of its C-terminal solubility tab (Lys^{P681}-Lys^{P684}) (FIG. 54). In addition, the side chain of Trp^{P678} has a different rotamer in peptide 104-2 in comparison to peptide 94-1 (FIG. 54B). This rotamer change is associated with crystal contacts unique to the 104-2 and 33-1 structures, which were determined using crystals belonging to the same space group (Table 8). In the 104-2 structure, Trp^{P678} has to adopt a different rotamer to avoid steric clashes with Leu^{H54} and Thr^{H55} of a molecule in another asymmetric unit. Interestingly, Trp^{P678} packs nicely against the tether of peptide 104-2 which would shield the backbone hydrogen bonds from solvent, potentially stabilizing the structure. These crystal contacts are also related to a rotamer change of Leu^{P679} in the 104-2 and 33-1 structures in comparison with the 94-1 structure. The rotamer change brings Leu^{P679} closer to Thr^{H31} in the 104-2 and 33-1 structures and closer to Ile^{H52} in the 94-1 structure (Table 9). Both Trp^{P678} and Leu^{P679} have clear electron density.

The helical conformation of the 4E10 epitope creates an amphipathic structure with a small polar face (defined by residues Asn^{P671}, Asp^{P674}, Asn^{P677}, and Tyr^{P681}) and a large hydrophobic face (Trp^{P672}, Phe^{P673}, Ile^{P675}, Thr^{P}676, Trp^{P678}, Leu^{P679}, Trp^{P680}, and Ile^{P682}) The epitope residues with the large number of contacts with antibody 4E10 are located on the hydrophobic face, suggesting that this is the “neutralizing face” of the epitope. The polar face of the epitope has crystal contacts with the H2 loop and the peptide molecule of the other antibody:epitope complex in the unit cell and, in the context of the virus, this “non-neutralizing face” for 4E10 could be involved in interactions with the viral membrane and/or other regions of gp41.

### The Combining Site.

The Fab 4E10 combining site is a largely hydrophobic cavity that is well adapted for binding of poorly water-soluble peptides. The surface area buried by the peptide on the Fab is 654 Å², 654 Å², and 610 Å² for peptides 94-1, 104-2, and 33-1, respectively. The corresponding buried surface area on the peptides is 625 Å², 617 Å², and 573 Å². In all three antibody-peptide complexes, 4E10 uses five of its six CDR loops to bind the peptide; CDR L2 is not used and CDR L1 makes only minor contacts. This pattern of CDR preferential usage and the size of the buried surface area are typical for anti-peptide antibodies (Stanfield et al., 1999).

A total of 117, 128, and 129 van der Waal's contacts are made between Fab 4E10 and peptide 94-1, 104-2, and 33-1, respectively (Table 9). Furthermore, several hydrogen bonds are made between rE10 and each peptide (Table 10). In the 104-2 complex, an additional hydrogen bond is made between the O_{γ1} of Orn^{P677} and the side chain of Lys^{L32}. However, antibody interactions with the key residues Trp^{P672}, Phe^{P673}, Ile^{P675}, and Thr^{P676} are basically preserved in all of four known 4E10-peptide complexes. The side chains of Trp^{P672} and Phe^{P673} are buried in the antibody-combining site and are involved in aromatic π-stacking interactions with 4E10 residues Trp^{P672} hydrogen bond to Ser^{L94} and Ile^{H56}, respectively (Table 10). The hydroxyl of Thr^{P676} hydrogen bonds to the carboxylate of Glu^{H95} (Table 10 and FIG. 55A) and Ile^{P675} stacks with CDR H2 residues Ile^{H52}, Leu^{H54}, and Ile^{H56} (Table 9 and FIG. 55C) to create a cluster of isoleucines/leucines on the edge of the antibody-combining site.

Antibody 4E10 binds with approximately 10³-fold higher affinity to peptides 94-1, 104-2, and 33-1 than to the original peptide KGND (Table 7), as determined by surface plasmon resonance and ELISA. This substantially increased affinity of 4E10 is likely due to the inclusion of appropriate flanking residues, such as Leu^{P679} and Trp^{P680}, in the re-designated peptides. The indole of Trp^{P680} hydrogen bonds to the backbone of Leu^{H100C}(Table 10 and FIG. 55C), as previously predicted (Cardoso et al., 2005), and the side chain of Leu^{P679} stacks with the side chains of Ile^{H52}, Leu^{H53}, and Leu^{H54}, extending the cluster of isoleucines/leucines created by Ile^{P675}, Ile^{H52}, Leu^{H54}, and Ile^{H56} on the edge of the antibody-combining site (Table 9 and FIG. 55C). Antibody 4E10 binds to peptides 94-1 and 104-2 with about 17-fold higher affinity than to peptide 33-1 (Table 7). In the case of peptide 94-1, this increased affinity could be related to the unique contacts between Leu^{P679} and Ile^{H52}, as well as the additional Tyr^{P681} and Lys^{H100E} interaction. In the case of peptide 104-2, the higher affinity in comparison to peptide 33-1 could be due to the hydrogen bond between Orn^{P677} and Lys^{L32}, which is seen only in peptide 104-2 (Tables 55 and 56). In contrast, the extra methyl group of Aib in peptide 33-1 does not interact with the antibody.

Constraining the peptide increased the binding affinity of the peptide epitope for 4E10. Replacement of Trp^{P678} with Aib increased in 3-fold the binding affinity of the peptide epitope 33-1 in comparison with its peptide control 44-1 (Table 7), as determined by ELISA. The tethered linkage in positions 674 and 677 resulted in a 4-fold increased affinity of the peptide epitope 104-2 in comparison with its peptide control 104-1 (Table 7). The on-rates of the two constrained peptides (33-1 and 104-2) are very similar (k″ values on Table 7), which is in agreement with similar peptide analogs previously characterized (Brunet et al., 2006). The difference in kd between peptides 104-2 and 33-1 (or 94-1) is due to a difference in k^{off} values, which are related to a better stabilization of the complex antibody:peptide. In the case of peptide 104-2 this stabilization could be because the additional H bond between Orn^{P677} and Lys^{L32}. The unconstrained peptide 94-1 presents faster on and off rates compared to 104-2, even though their K^{d} values are similar. Constraining the peptide did not facilitate the formation of the complex with the antibody but it increased the stability of the complex once formed.

Tyr^{P681}, Ile^{P682}, and Lys^{P683}, the gp41 residues immediate prior to the transmembrane domain, do not have close contacts with 4E10. However, these residues extend the helix, which suggests the 4E10 epitope may form a continuous helix with the transmembrane domain. As observed in the 4E10:peptide 94-1 complex, only the O_{H} and C_{ξ2} atoms of Tyr^{P681} have a 3.9 and 4.2 van der Waals' interaction, respectively, with the Cd atom of Lys^{H100E}, a residue located near the base of the antibody H3 loop. However, Tyr^{P681} and Ile^{P682} make intra-peptide contacts that might have a structural role in maintaining the side chain orientation of epitope residues contacting the antibody (FIGS. 55B and 55C). Residue Tyr^{P681} stacks with Trp^{P680}, a key epitope residue for 4E10 binding, and could help to stabilize the Trp^{P680} in an optimal conformation for interaction with the antibody (FIG. 55B). The side chain of Ile^{P682}, which has no contacts with 4E10, packs with Leu^{P679} and Ile^{P675} (FIG. 55C) to expand the cluster of Ile/Leu at the edge of antibody combining site.

### Expansion of the Core Epitope

The structural analysis of the contributions made by each peptide residue to 4E10 binding reveals the key epitope residues. In the present Example, the crystal structures of 4E10 bound to longer peptides (encompassing region 671-683 of gp41) reveal that an additional helical turn including Leu^{P679} and Trp^{P680}, in addition to the previously defined residues Trp^{P682}, Phe^{P673}, Ile^{P675}, and Thr^{P676}, make a significant number of selective contacts with 4E10 (FIG. 55, Tables 9 and 10). These findings complement and extend the results obtained from the previous 4E10 structure along with mutagenesis and binding experiments (Cardoso et al., 2005; Zwick et al., 2005; Brunel et al., 2006). Thus, the 4E10 core epitope has been extended to the contiguous motif WFX(I/L)(T/S)XX(L/I)W. Selective contacts with these key residues in a helical conformation dictate the high affinity for 4E10 for the epitope. Furthermore, the fact that the key epitope residues are very conserved in all HIV-1 viruses explains the broadly neutralizing activity of antibody 4E10. Trp^{P682}, Ile^{P675}, and Leu^{P679} are all highly conserved residues on gp41 from different HIV-1 subtypes. Phe^{P673} is replaced by Leu in only a few isolates. Thr^{P676} can be replaced by a serine, which is found in many HIV isolates that are neutralized by 4E10. The tolerance of the 4E10 epitope to both Thr and Ser at the 676 position could be due to the maintenance of the hydrogen bond with CDR H3 residue Glu^{H95}. Similarly, Trp^{P680} is occasionally replaced by Arg, which can maintain the hydrogen bond interaction with the carbonyl oxygen of Leu^{H100C}, as previously observed for Lys^{P680} in the crystal structure of peptide KGND bound to 4E10.

### DISCUSSION AND CONCLUSION

Elucidation of the critical features of 4E10 recognition of HIV-1 helps to define potential immunogens able to elicit 4E10-like antibodies. Antibody 4E10 recognizes a contiguous and helical WFX(I/L)(T/S)XX(L/I)W motif, where X does not play a major role in 4E10 contacts. Crystal structures of 4E10 bound to peptide epitopes (encompassing region 671-683 of gp41) reveal that the gp41 residues Trp⁶⁸², Phe⁶⁷³, Ile⁶⁷⁵, Thr⁶⁷⁶, Leu⁶⁷⁹, and Trp⁶⁸⁰ have the most significant contacts with the antibody. On the other hand, the “X” residues potentially can stabilize the helical structure in solution (Brunel et al., 2005) and can be used to introduce conformational constraints.

An effective immunogen needs to present a single, stable conformational epitope to the immune system. Although gp41 peptides based on the 4E10 epitope are helical in solution, these linear peptides could adopt numerous alternative conformations when bound to an antibody. As a result, simple linear peptides elicit non-neutralizing antibodies. In order to stabilize the helical conformation and also destabilize alternative conformations, peptidomimetic constraints were introduced at the non-interacting “X” positions of the eptiope. The first approach was to substitute Aib, an unnatural amino acid, at position 678 in the gp41 sequence. Aib residues have two methyl groups bound to the C_{α} atom, which restrict the backbone to the helical region of the Φ,Ψ dihedral angle map (Marshall et al., 1990) and can stabilize both 3₁₀ and α-helices while extended conformations are destabilized. Aib-containing peptides bound to antibodies has been previously structurally characterized with the Aib residue in a constrained beta turn conformation (PDB entries 1AI1 and 1F58; Ghiara et al., 1997 and Stanfield et al., 1999). In this study, the structure of the 33-1:4 E10 complex shows that the bound Aib peptide has phi/psi angles in the α-helical region, nearly identical to the unconstrained peptide 94-1. In addition, the side chain residues contacting the antibody are nearly identical—the rmsd of superpositions between 33-1 and 94-1 is just 0.6 Å (superposition of the C_{β} of all peptide residues and the side chains of only residues contacting 4E10). Importantly, the Aib side chain makes no significant contacts with the antibody or other peptide side chains.

A second approach was the lock the helix with a tether link between positions 674 and 677 in the 104-2 peptide. This 4E10 bound peptide epitope has the expected helical conformation and, similar to the Aib-containing peptide, the side chain residues contacting the antibody adopts rotamers nearly identical to the unconstrained peptide 94-1-the rmsd of backbone superpositions, including C_{β} of all residues and side chains of contacting residues, between 104-2 and 4-1 is only 0.7 Å. However, the tether link forms a hydrogen bond with the antibody (between the O_{γ1} of Orn^{P677} and the side chain of Lys^{L32}) and Trp^{P678} has a rotamer that packs against the tether. While extra interactions are usually desirable for drug design, they represent a “red flag” for immunogen design because they could elicit non-neutralizing antibodies to these new elements. Thus, this long tether link might not be the most appropriate constraint for the immunogen and perhaps a shorter tether loop will have a better fit. Furthermore, peptides encompassing the 4E10 epitope (down to the YIK motif) with an Aib replacing Asp⁶⁷⁴ as well as maybe Asn⁶⁷⁷ and/or Trp⁶⁷⁸ may be part of the next generation of immunogens.

The CDR H3 of 4E10 remains something of an enigma and a potential interaction of this CDR with the viral membrane is another source of considerations for the immunogen design. The CDR H3 of 4E10, as for antibody 2F5, has a large surface remaining that is not involved in antigen contact. A more typical situation has the CDR H3 in contact with antigen throughout most of its length (MacCallum et al., 1996). The length (18 residues), extensive area not contacting the epitope, hydrophobic character, and glycine-rich composition of the CDR H3 of 4E10 raises the possibility that the tip of the H3 loop, particularly Trp^{H100} and Trp^{H100B}, has further interactions with the viral membrane or with other gp41 or gp120 residues, in the context of the intact virus. Biochemical analysis using envelope glycoprotein proteoliposomes suggests that 4E10 and 2F5 binding is enhanced in the presence of a lipid membrane (Ofek et al., 2004). Mutagenesis studies of the H3 loop of the 4E10 are ongoing to test the importance of the CDR H3 for 4E10 binding to gp41 and virus neutralization. Presentation of the 4E10 epitope as an oligomer and/or in a membrane-like context should be further evaluated.

To develop an effective immunogen to elicit 4E10-like antibodies, a multi-step strategy was adopted. Initially, the extension and properties of the 4E10 minimal epitope (the contiguous and helical WFX(I/L)(T/S)XX(L/I)W motif were identified and characterized atomic detail. A 10³-fold increase in binding affinity was achieved by 4E10 interaction with an additional helical turn at the C-terminus that includes Leu⁶⁷⁹ and Trp⁶⁸⁰, gp41 residues shown to contact CDRs H2 and H3 of 4E10. Next, constraints were introduced towards the C-terminal of the epitope to increase the helical character in this region of the peptide. Constrained peptides are better immunogen candidates as they cannot adopt some conformations which would only elicit non-neutralizing antibodies. Introduction of Aib in position 678 of the peptide epitope or a tether bridge between residues 674 and 677 resulted in a 3₁₀ helix (residues WF) followed by an α-helix (residues (I/L)(T/S)XX(L/I)W) structure, which is also observed for the non-contrained peptide epitope. While the Aib side chain makes no additional contacts with the antibody, the tether link has undesirable extra interactions with 4E10, which could contribute to elicitation of non-neutralizing antibodies. The next generation of immunogens will have dispensable constituents of the 4E10 epitope replaced with less immunogenic substituents to mask the “non-neutralizing face” without perturbing the contrained helical conformation. This step will pursue presentation of only the face of the helix contacting 4E10 to the immune system. Additionally, a helical presentation of the core 4E10 epitope in a membrane-like context, for instance liposomes, may have a major impact on the design of a vaccine candidate to elicit 4E10-like antibodies.

### Accession Numbers

Coordinates and structure factors for Fab 4E10 bound to peptides 94-1, 33-1 and 104-2 have been deposited in the Protein Data Bank under accession codes 2FX7, 2FX8, and 2FX9, respectively.

### REFERENCES

U.S. Pat. No. 4,196,265
U.S. Pat. No. 6,037,117
U.S. Pat. No. 6,077,682
U.S. Pat. No. 6,087,478
U.S. Pat. No. 6,110,672
U.S. Pat. No. 6,128,582
U.S. Pat. No. 6,153,579
U.S. Pat. No. 6,221,645
U.S. Ser. No. 10/471,328
WO 00/47763
WO 01/37194
WO 02/0222793
WO 02/077270
WO 02/079490
WO 02/095035
WO 03/012089
WO 03/022879
WO 03/035693
WO 98/57155
WO 99/45379
Afonin et al. (2001). Protein Sci. 10, 1514-1521.
Al-Lazikani et al. (1997). J. Mol. Biol. 273, 927-948.
Altschul and Gish, 1996, Local alignment statistics, Doolittle ed., Methods in Enzymology 266: 460-480
Altschul et al., 1990, J. Mol. Biol. 215: 403-410
Barbato et al. (2003) J. Mol. Biol. 330, 1101-1115.
Binley et al. (2003) J. Virol. 77, 5678-5684.
Binley et al. (2004). J. Virol. 78, 13232-13252.
Biron et al. (2002) Biochemistry 41, 12687-12696.
Bjorndal et al. (1997) J. Virol. 71-7478-87.
Blundell et al. Eur J Biochem 172 (1988), 513
Blundell, in Protein Crystallography, Academic Press, NY, London and San Francisco (1976)
Bohacek et al., Medicinal Research Reviews, 16 (1996), 3-5.
Brunel & Dawson (2005) Chem. Comm. 20, 2552-2554.
Brunel et al. (2003a) Letters in Peptide Science 9:111-17.
Brunel et al. (2003b) Tetrahedron Lett. 44:1287-89.
Brunel et al. (2006) J. Virol. 80, 1680-1687.
Brünger et al. (1998) Acta Crystallogr. D54, 905-921.
Buchacher et al. (1994) AIDS Res. Hum. Retroviruses 10, 359-369.
Bullough et al. (1994) Nature 371, 37-43.
Burton (1997) Proc. Natl. Acad. Sci. USA. 94(19):10018-23.
Burton et al. (2004) Nat. Immunol. 5, 233-236.
Burton et al. (1994) Science 266, 1024-1027.
Burton et al. (2005) Proc. Natl. Acad. Sci. USA 102, 14943-14948.
Caffrey et al. (1998) EMBO J. 17, 4572-4584.
Calarese et al. (2003) Science 300, 2065-2071.
Cardoso et al. (2005) Immunity 22, 163-73.
Chan & Kim (1998) Cell, 93, 681-684.
Chan et al. (1997) Cell 89, 263-273.
Chen et al. (1999) Proc. Natl. Acad. Sci. USA 96, 8967-8972.
Coeffier et al. (2000) Vaccine 19, 684-93.
Collaborative computational project number 4. (1994) The CCP4 suite: Programs for protein crystallography. Acta Crystallogr. D50, 760-763.
Conley et al. (1994) Proc. Natl. Acad. Sci. USA 91, 3348-3352.
Connolly (1993) J. Mol. Graphics. 11, 139-141.
Connor et al. (1995) Virology 206, 935-44.
Cotton & Wilkinson, Inorganic Chemistry (John Wiley & Sons, Fourth Ed. 1980), esp. Ch. 2
Darbha et al. (2004) Biochemistry 43, 1410-1417.
DeLano (2002). The PyMOL Molecular Graphics System. DeLano Scientific, San Carlos, Calif., USA.
Dimitrov et al. (2003) Biochemistry 42, 14150-14158.
Dunbrack et al. Folding and Design 2 (1997), 27-42
Eckert et al. (1999) Cell 99, 103-115.
Ernst et al. (2003) Angew. Chem. Int. Ed. Engl. 42, 535-539.
Ferrantelli & Ruprecht (2002). Curr. Opin. Immunol. 14, 495-502.
Ferrantelli et al. (2003) AIDS 17, 301-309.
Ferrer et al. (1999) Nat. Struct. Biol. 6, 953-960.
Gallo et al. (2003). Bioch. Bioph. Acta 1614, 36-50.
Gauduin et al. (1997) Nat. Med. 3, 1389-1393.
Gelin and Karplus (1979) Biochemistry 18, 1256-1268.
Ghiara et al. (1997) J. Mol. Biol. 266, 31-39.
Gish and States, (1993) Nature Genetics 3: 266-272
Gordon et al. (2004) Protein Sci. 13, 1012-1030.
Goodford (1985) J. Med. Chem., 28, 849-57
Greer et al. (1994) J of Medicinal Chemistry 37, 1035-54
Greer (1985) Science 228, 1055
Hutchinson and Thornton (1996) Protein Sci. 5, 212-220.
Jackson et al. (1991) J. Am. Chem. Soc. 113, 9391-9392.
Ji et al. (1999)J. Virol. 73, 8578-8586.
Jiang & Debnath (2000) Biochem. Biophys. Res. Commun. 269, 641-646.
Jiang et al. (1993) Nature 365, 113.
Jiang et al. (1993) Biochem. Biophys. Res. Commun. 195, 533-538.
Jones et al. (1995) Current Opinion in Biotechnology 6, 652-656
Jones et al. (1991) Acta Crystallogr. A47, 110-119.
Joyce et al. (2002) J. Biol. Chem. 277(48):45811-20
Kabat et al. (1991). Sequences of proteins of immunological interest. (U.S. Department of health and human services).
Karlin and Altschul (1990) Proc. Natl. Acad. Sci. USA 87: 2264-2268
Karlin and Altschul, (1993), Proc. Natl. Acad. Sci. USA 90: 5873-5877
Kitabwalla et al. (2003) AIDS Res. Hum. Retroviruses 19, 125-31.
Kleywegt & Jones Halloween . . . Masks and bones! in From First Map to Final Model (eds Bailey, S., Hubbard, R. & Waller, D.) pp. 59-66, SERC Darsbury Laboratory, Warrington, UK, (1994).
Kochendoerfer (2001), Current Opinion in Drug Discovery and Development 4, 205-214
Kraulis (1991) J. Appl. Crystallogr. 24, 946-950.
Ktabwalla et al. AIDS Res Hum Retroviruses 19(2):125-31 (2003)
Kunert et al. (2000) Biotechnol. Bioeng. 67, 97-103.
Kunert et al. (2004) AIDS Res. Hum. Retroviruses 20, 755-762.
Kuszewski et al. (1999) J. Am. Chem. Soc. 121, 2337-2338.
Kutzki et al. (2002) J. Chem. Soc. 124, 11838-11839.
Kwong et al. (1998) Nature 393, 648-659.
Leduc et al. (2003) PNAS 100, 11273-11278.
Liu et al. (2001) Biochemistry 40, 2797-2807.
Liu et al. (2002). J. Biol. Chem. 277, 12891-12900.
Lu et al. (2001) J. Virol. 75, 11146-11156.
MacCallum et al. (1996) J. Mol. Biol. 262, 732-745.
Malashkevich et al. (1998) Proc. Natl. Acad. Sci. USA 95, 9134-9139.
Malashkevich et al. (1999) Proc. Natl. Acad. Sci. USA 96, 2662-2667.
Malashkevich et al. (2001) Proc. Natl. Acad. Sci. USA 98, 8502-8506.
Maniatis et al. (1982) Molecular Cloning: A Laboratory Manual.
Marshall et al. (1990) PNAS 87, 487-491.
Mascola (2003) Curr. Mol. Med. 3, 209-216.
Mascola et al. (2000) Nat. Med. 6, 207-210.
McDonald and Thornton (1994). J. Mol. Biol. 238, 777-793.
McGaughey (2003) Biochemistry 42(11):3214-23
McPherson (1982) Preparation and Analysis of Protein Crystals, John Wiley and Sons, New York.
McPherson (1990) Eur J Biochem. 189(1):1-23.
McRee (1999) J. Struct. Biol. 125, 156-165.
Merritt and Bacon (1997) Meth. Enzymol. 277, 505-524.
Munoz-Barroso et al. (1999) J. Virol. 73, 6089-6092.
Muster et al. (1993) J. Virol. 67, 6642-6647.
Myers and Miller (1988) CABIOS 4: 11-17
Navaza (1994) Acta Crystallogr. A50, 157-163.
Nicholls et al. (1991) Proteins 11, 281-296.
Ofek et al (2004) J. Virol. 78, 10724-10737.
Otwinowski & Minor (1997) Meth. Enzymol. 276A, 307-326.
Parren et al. (2001) J. Virol. 75, 8340-8347.
Pearson and Lipman (1988) Proc. Natl. Acad. Sci. USA 85: 2444-2448
Pessi et al., (2003) J. Mol. Biol. 5:1201-15
REMINGTON′S PHARMACEUTICAL SCIENCE″, 17th edition, 1985
Rimsky et al. (1998) J. Virol. 72, 986-993.
Ruprecht et al. (2003) Vaccine 21, 3370-3373.
Salzwedel et al. (1999). J. Virol. 73, 2469-2480.
Saphire et al. (2001) Science 293, 1155-1159.
Sayle et al. (1995) TIBS 20, 374
Schibli et al. (2001) Biochemistry 40, 9570-9578.
Schnolzer et al. (1992) Int. J. Pept. Protein Res. 40: 180-93.
Scott & Smith (1990) Science 249, 386-390.
Sheriff, et al. (1987). J. Mol. Biol. 197, 273-296.
Shibata et al. (1999) Nat. Med. 5, 204-210.
Shu et al. (2000b) J. Biol. Chem. 275, 1839-1845.
Shu et al. (2000a) Biochemistry 39, 1634-1642.
Skuker et al. (1996) Science 274, 1531-1534
Stanfield et al. (1999) Struct. Fold. Des. 7, 131-142.
Stanfield & Wilson, (1995) Curr. Opin. Struct. Biol. 5, 103-113.
Stanfield et al. (2004) Structure 12, 193-204.
Stiegler et al. (2001) AIDS Res. Hum. Retroviruses 17, 1757-1765.
Stout et al. (1998) Structure 6, 839-48
Suarez et al. (2000) J. Virol. 74, 8038-8047.
Tan et al. (1997) Proc. Natl. Acad. Sci. USA 94, 12303-12308.
van Den Elsen et al. (1999) Proc. Natl. Acad. Sci. USA 96, 13679-13684.
Verlinde et al. (1992) J of Computer-Aided Molecular Design 6, 131-147
Walensky et al. (2004) Science 305, 1466-1470.
Walters et al. (1998) Drug Discovery Today, 3(4):160-178
Weber et al. (1991) Advan Protein Chem. 41:1-36.
Wei et al. (2002) Antimicrob. Agents Chemother. 46, 1896-905.
Weissenhorn et al. (1998) Mol. Cell. 2, 605-616.
Weissenhorn et al. (1997) Nature 387, 426-430.
Wilbur and Lipman (1983) PNAS USA 80:726
Wild et al. (1994) Proc. Natl. Acad. Sci. USA 91, 9770-9774.
Wilson et al. (1981) Nature 289, 366-373.
Wyatt and Sodroski (1998) Science 280, 1884-1888.
Yang et al. (1999) J. Struct. Biol. 126, 131-144.
Zhou et al. (2000) Bioorg. Med. Chem. 8, 2219-2227.
Zwick et al. (2001a) J. Virol. 75, 10892-10905.
Zwick et al. (2001b) J. Virol. 75, 12198-12208.
Zwick et al. (2003) J. Virol. 77: 6965-78.
Zwick et al. (2005) J. Virol. 79, 1252-1261.

The invention may be described by the following numbered paragraphs:

1. A Fab 4E10:KGND complex having the crystal structure herein described, comprising a C2 space group, cell parameters (in angstroms for a, b, c and degrees for Beta, rms deviations 0.005 angstroms, 1.3 degrees) of a: 157.3 angstroms, b: 45.1 angstroms, c: 198.6 angstroms, and Beta: 113.8 degrees and/or having an X-ray diffraction pattern corresponding to or resulting from any or all of the foregoing and/or having an X-ray diffraction pattern corresponding to or resulting from any or all of the foregoing and/or a crystal having the structure defined by the co-ordinates of Table 1.

2. A method for screening or identification comprising exposing the Fab 4E10 of the foregoing crystal structure to one or more test samples, and determining whether a Fab 4E10 complex is formed.

3. The method of paragraph 2 performed wherein the Fab 4E10 or functional portion thereof is exposed to the test samples by co-crystallizing the Fab 4E10 protein or functional portion thereof in the presence of the one or more test samples.

4. The method of paragraph 2 or 3 wherein resulting crystals are analyzed by X-ray diffraction or crystallographic techniques and compared with the herein data, wherein if similar in crystal structure, the test sample thus binds to Fab 4E10 in a manner analogous to KGND, and is thus useful for eliciting antibodies or in a diagnostic, pharmaceutical immunogenic, immunological or vaccine composition; optionally, the Fab 4E10 can be soaked in a solution of one or more test samples.

5. A computer-assisted method for identifying or designing potential compounds to fit within or bind to Fab 4E10 or a functional portion thereof:
comprising using a computer system comprising a processor, a data storage system, an input device, and an output device, the steps of: (a) inputting into the programmed computer through said input device data comprising the three-dimensional co-ordinates of a subset of the atoms in the Fab 4E10 binding domain (containing or binding to key residues identified herein), optionally with structural information from Fab 4E10 complex(es), such as the Fab 4E10:KGND complex, thereby generating a data set; (b) comparing, using said processor, said data set to a computer database of chemical structures stored in said computer data storage system; (c) selecting from said database, using computer methods, chemical structures having a portion that is structurally similar to said data set; (d) constructing, using computer methods, a model of a chemical structure having a portion that is structurally similar to said data set and (e) outputting to said output device the selected chemical structures having a portion similar to said data set; and optionally synthesizing one or more of the selected chemical structures; and further optionally contacting said synthesized selected chemical structure with Fab 4E10 to ascertain whether said synthesized chemical structure binds to or fits within the domain of Fab 4E10 and/or administering said chemical structure to an animal capable of having an antibody response to ascertain whether the chemical structure elicits anti-HIV antibodies by testing said resultant antibodies for binding to HIV or HIV glycoproteins or portions thereof; or,
comprising: providing the structure of Fab 4E10 as defined by the co-ordinates of Table 1, providing the structure of a candidate binding molecule, and fitting the structure of the candidate to the structure of the Fab 4E10 of Table 1; or,
comprising: providing the co-ordinates of at least two atoms of Table 1 of Fab 4E10 (“selected co-ordinates”), providing the structure of a candidate binding molecule, and fitting the structure of the candidate to the selected co-ordinates; or,
comprising: providing the co-ordinates of at least a sub-domain of Fab 4E10, providing the structure of a candidate binding molecule, and fitting the structure of the candidate to the sub-domain of Fab 4E10;
said method optionally further comprising: obtaining or synthesizing the chemical structure or candidate and contacting the chemical structure or candidate with Fab 4E10 to determine the ability of the chemical structure or candidate to interact with Fab 4E10; or obtaining or synthesizing the chemical structure or candidate and forming a complex of Fab 4E10 and said chemical structure or candidate, and analyzing the complex to determine the ability of said chemical structure or candidate to interact with Fab 4E10 and/or administering said chemical structure or candidate to an animal capable of raising antibodies against the chemical structure to ascertain whether said chemical structure or candidate elicits anti-HIV antibodies comprising testing said resultant antibodies for binding to HIV or HIV glycoproteins or portions thereof.

6. A method of transmitting data comprising transmission of information via telecommunication, telephone, video conference, mass communication, computer presentation, interne, email, documentary communication such as a computer program document and the like.

7. A compound having a chemical structure selected using the method of any one of paragraphs 2 to 6, said compound binding to Fab 4E10 and eliciting an anti-HIV antibody.

8. A diagnostic/pharmaceutical/immunogenic/immunological/vaccine composition composition containing a compound of paragraph 7.

9. A method for making a composition comprising a compound according to paragraph 7 or 8, wherein the method comprises admixing such compound with a pharmaceutically suitable or acceptable vehicle or carrier or diluent, optionally including or being an adjuvant.

10. A method for using a composition according to paragraph 8 wherein the compositions is administered to an animal that generates antibodies to the compound or composition, wherein the antibodies generated are anti-HIV antibodies that may be diagnostically useful or wherein administration of the composition elicits an immunogenic or immunological or vaccine response; or, wherein the compound is used detect the presence of anti-HIV antibodies in a sample.

11. A method of eliciting anti-HIV antibodies comprising administering to an animal capable of eliciting antibodies a compound or composition of paragraph 7 or 8.

12. A method for detecting anti-HIV antibodies comprising contacting a sample suspected of having such antibodies with a compound of paragraph 7, and detecting binding.

13. The method of paragraph 11 wherein the animal is a human and the method is for treatment or prevention of HIV.

14. The method of paragraph 11 wherein the method is for generating antibodies for diagnostic purposes.

15. A diagnostic composition containing a compound of paragraph 7, or an antibody elicited by administration of said composition or compound.

16. A composition for prevention or treatment of HIV comprising a compound paragraph 7, or an antibody elicited by administration of said composition or compound.

17. A computer system for generating or performing rational compound design for Fab 4E10 complexes of Fab 4E10 with a potential binder, the system containing either: atomic coordinate data according to Table 1 and/or the Figures, said data defining the three dimensional structure of Fab 4E10 or at least one sub-domain thereof, or structure factor data for Fab 4E10, said structure factor data being derivable from the atomic co-ordinate data of Table 1 and/or the Figures.

18. A computer readable media containing either: atomic co-ordinate data according to Table 1 and/or the Figures, said data defining the three dimensional structure of Fab 4E10 or at least one sub-domain thereof, or structure factor data for Fab 4E10, said structure factor data being derivable from the atomic co-ordinate data of Table 1 and/or the Figures.

19. A method of doing business comprising providing to a user the computer system of paragraph 17 or the media of paragraph 18 or the three dimensional structure of Fab 4E10 or at least one sub-domain thereof, or structure factor data for Fab 4E10, said structure set forth in and said structure factor data being derivable from the atomic co-ordinate data of Table 1 and/or the Figures.

20. A method of preparing a compound comprising chemically synthesizing said compound, wherein said compound is a peptide mimic of KGND, or is a compound of Table 4.

21. A compound as in paragraph 7, comprising a peptide mimic of KGND, wherein there is one or more conservative substitutions of amino acids of KGND for the peptide mimic.

22. A polypeptide herein described as KGND having the sequence as shown in FIG. 9 or as described in the brief description of FIG. 9.

23. A derivative or homologue of the polypeptide of paragraph 22.

24. A polypeptide having at least 50 percent homology with the polypeptide of paragraph 22.

25. A polypeptide having at least 60 percent homology with the polypeptide of paragraph 22.

26. A polypeptide having at least 70 percent homology with the polypeptide of paragraph 22.

27. A polypeptide having at least 75 percent homology with the polypeptide of paragraph 22.

28. A polypeptide having at least 80 percent homology with the polypeptide of paragraph 22.

29. A polypeptide having at least 85 percent homology with the polypeptide of paragraph 22.

30. A polypeptide having at least 90 percent homology with the polypeptide of paragraph 22.

31. A polypeptide having at least 93 percent homology with the polypeptide of paragraph 22.

32. A polypeptide having at least 95 percent homology with the polypeptide of paragraph 22.

33. A polypeptide having at least 97 percent homology with the polypeptide of paragraph 22.

34. A polypeptide having at least 98 percent homology with the polypeptide of paragraph 22.

35. A polypeptide having at least 99 percent homology with the polypeptide of paragraph 22.

36. A polypeptide which consists essentially of WFXIT (SEQ ID NO: 78), wherein X may be N, D, S, G or other amino acids, including conservative substitutions thereof

37. The polypeptide of paragraph 36, wherein X may additionally be Aib or O.

38. The polypeptide of paragraph 36, wherein Aib may be inserted between any two amino acids of WFXIT (SEQ ID NO: 78).

39. The polypeptide of paragraph 36, wherein WFXIT (SEQ ID NO: 78) is branched.

40. The branched polypeptide of paragraph 36, wherein the branched chain is of sufficient length and/or configuration that the polypeptide binds to Fab 4E10.

41. A polypeptide having a sequence consisting essentially of DKWX¹X²X³X⁴X⁵WFXIT (SEQ ID NO: 3), wherein X is as defined above in paragraph 36, X¹=A or a conservative substitution thereof, X²=N or a conservative substitution thereof, X³=L or a conservative substitution thereof, X⁴=W or a conservative substitution thereof, X⁵=N, S or T or a conservative substitution thereof, wherein the polypeptide has a helical structure, and it is not otherwise disclosed in he art.

42. A polypeptide having a sequence consisting essentially of DKWX¹X²X³X⁴X⁵WFXIT (SEQ ID NO: 3), wherein

X=N, D, S, G, Q, C, T, M, E, K, R, A, P, I, L, V, O, Aib, or other natural or synthetic amino acids, including conservative substitutions thereof,

X¹=A, G, P, I, L, V, Aib, or other natural or synthetic amino acids, or a conservative substitution thereof;

X²=N, Q, C, S, T, M, or other natural or synthetic amino acids, or a conservative substitution thereof;

X³=L, I, V, G, A, P, or other natural or synthetic amino acids, or a conservative substitution thereof,

X⁴=W, H, F, Y, K, C, Aib, or other natural or synthetic amino acids, or a conservative substitution thereof,

X⁵=N, S, T, Q, C, M, E, A, or other natural or synthetic amino acids, or a conservative substitution thereof;

wherein the polypeptide has a helical structure, and it is not otherwise disclosed in the art.

43. The polypeptide of paragraph 42, wherein Aib may be inserted between any two amino acids of WFXIT (SEQ ID NO: 78).

44. The polypeptide of paragraph 42, wherein WFXIT (SEQ ID NO: 78) is branched.

45. The branched polypeptide of paragraph 44, wherein the branched chain is of sufficient length and/or configuration that the polypeptide binds to Fab 4E10.

46. The polypeptide of paragraph 42, wherein the polypeptide comprises or consists essentially of:

(SEQ ID NO: 19)
NWFDITNWLWRR-NH₂,
(SEQ ID NO: 20)
SLWNWFDITNWLWRR-NH₂,
(SEQ ID NO: 21)
DKWASLWNWFDITNWLWRR-NH₂,
(SEQ ID NO: 15)
NWFDITNWLWKKKK-NH₂,
(SEQ ID NO: 22)
WNWFDITNWLWKKKK-NH₂,
(SEQ ID NO: 23)
LWNWFDITNWLWKKKK-NH₂,
(SEQ ID NO: 24)
SLWNWFDITNWLWKKKK-NH₂,
(SEQ ID NO: 25)
NWFDITNWLAKKKK-NH₂,
(SEQ ID NO: 26)
WNWFDITNWLAKKKK-NH₂,
(SEQ ID NO: 27)
LWNWFDITNWLAKKKK-NH₂,
(SEQ ID NO: 28)
SLWNWFDITNWLAKKKK-NH₂,
(SEQ ID NO: 29)
Ac-WFDIT-Aib-NH₂,
(SEQ ID NO: 30)
Ac-NWFDIT-Aib-NH₂,
(SEQ ID NO: 31)
Ac-Aib-NWFDIT-Aib-NH₂,
(SEQ ID NO: 32)
Ac-DKWASL-Aib-NWFDIT-Aib-NH₂,
(SEQ ID NO: 33)
Ac-ELDKWASL-Aib-NWFDIT-Aib-NH₂,
(SEQ ID NO: 34)
NWFDITN-Aib-LWRR-NH₂,
(SEQ ID NO: 35)
SL-Aib-NWFDITN-Aib-LWRR-NH₂,
(SEQ ID NO: 36)
DKW-Aib-SL-Aib-NWFDITN-Aib-LWRR-NH₂,
(SEQ ID NO: 37)
Ac-CAWFO(Ac)IT-NH₂,
(SEQ ID NO: 38)
Ac-c(CAWFO)IT-NH₂,
(SEQ ID NO: 39)
CAWFO(Ac)IT-NH₂,
(SEQ ID NO: 40)
c(CAWFO)IT-NH₂,
(SEQ ID NO: 41)
KKCAWFO(Ac)IT,
(SEQ ID NO: 42)
Ac-KKc(CAWFO)IT-NH₂,
(SEQ ID NO: 43)
c(CNWFO)ITNWLWRR-NH₂,
(SEQ ID NO: 44)
CNWFO(Ac)ITNWLWRR,
(SEQ ID NO: 45)
DKWASLc(CNWFO)ITNWLWRR-NH₂,
(SEQ ID NO: 46)
DKWASLCNWFO(Ac)ITNWLWRR-NH₂,
(SEQ ID NO: 47)
LELDKWASLc(CNWFO)ITNWLWRR-NH₂,
(SEQ ID NO: 48)
LELDKWASLCNWFO(Ac)ITNWLWRR-NH₂,
(SEQ ID NO: 49)
CWFOITNWLWKK-NH₂,
(SEQ ID NO: 50)
CWFOITNWLWKK-NH₂,
(SEQ ID NO: 51)
WCWFOITNWLWKK-NH₂,
(SEQ ID NO: 52)
CWFOITNWLWKKKK-NH₂,
(SEQ ID NO: 53)
c(CWFO)ITNWLWKKKK-NH₂,
(SEQ ID NO: 54)
WCWFOITNWLWKKKK-NH₂,
(SEQ ID NO: 55)
Wc(CWFO)ITNWLWKKKK-NH₂,
(SEQ ID NO: 56)
NWFEITNKLWGRRRRC,
(SEQ ID NO: 57)
NWFc(EITNK)LWGRRRRC,
(SEQ ID NO: 58)
LWNWFEITNKLWGRRRRC,
(SEQ ID NO: 59)
LWNWFc(EITNK)LWGRRRRC,
(SEQ ID NO: 60)
DKWASLWNWFEITNKLWGRRRRC,
(SEQ ID NO: 61)
DKWASLWNWFc(EITNK)LWGRRRRC,
(SEQ ID NO: 62)
LLELDKWASLWNWFEITNKLWGRRRRC,
(SEQ ID NO: 63)
LLELDKWASLWNWFc(EITNK)LWGRRRRC,
(SEQ ID NO: 64)
NWFEITNWLWGRRRRC,
(SEQ ID NO: 65)
DKWASLKNWFEITNWLWGRRRRC,
(SEQ ID NO: 66)
DKWASLc(KNWFE)ITNWLWGRRRRC,
(SEQ ID NO: 67)
LLELDKWASLKNWFEITNWLWGRRRRC,
(SEQ ID NO: 68)
LLELDKWASLc(KNWFE)ITNWLWGRRRRC,
(SEQ ID NO: 69)
EWFKITNWLWKKKK-NH₂,
(SEQ ID NO: 70)
c(EWFK)ITNWLWKKKK-NH₂,
(SEQ ID NO: 71)
WEWFKITNWLWKKKK-NH₂,
or
(SEQ ID NO: 72)
Wc(EWFK)ITNWLWKKKK-NH₂.

47. A polypeptide comprising or consisting essentially of:

(SEQ ID NO: 19)
NWFDITNWLWRR-NH₂,
(SEQ ID NO: 20)
SLWNWFDITNWLWRR-NH₂,
(SEQ ID NO: 21)
DKWASLWNWFDITNWLWRR-NH₂,
(SEQ ID NO: 15)
NWFDITNWLWKKKK-NH₂,
(SEQ ID NO: 22)
WNWFDITNWLWKKKK-NH₂,
(SEQ ID NO: 23)
LWNWFDITNWLWKKKK-NH₂,
(SEQ ID NO: 24)
SLWNWFDITNWLWKKKK-NH₂,
(SEQ ID NO: 25)
NWFDITNWLAKKKK-NH₂,
(SEQ ID NO: 26)
WNWFDITNWLAKKKK-NH₂,
(SEQ ID NO: 27)
LWNWFDITNWLAKKKK-NH₂,
(SEQ ID NO: 28)
SLWNWFDITNWLAKKKK-NH₂,
(SEQ ID NO: 29)
Ac-WFDIT-Aib-NH₂,
(SEQ ID NO: 30)
Ac-NWFDIT-Aib-NH₂,
(SEQ ID NO: 31)
Ac-Aib-NWFDIT-Aib-NH₂,
(SEQ ID NO: 32)
Ac-DKWASL-Aib-NWFDIT-Aib-NH₂,
(SEQ ID NO: 33)
Ac-ELDKWASL-Aib-NWFDIT-Aib-NH₂,
(SEQ ID NO: 34)
NWFDITN-Aib-LWRR-NH₂,
(SEQ ID NO: 35)
SL-Aib-NWFDITN-Aib-LWRR-NH₂,
(SEQ ID NO: 36)
DKW-Aib-SL-Aib-NWFDITN-Aib-LWRR-NH₂,
(SEQ ID NO: 37)
Ac-CAWFO(Ac)IT-NH₂,
(SEQ ID NO: 38)
Ac-c(CAWFO)IT-NH₂,
(SEQ ID NO: 39)
CAWFO(Ac)IT-NH₂,
(SEQ ID NO: 40)
c(CAWFO)IT-NH₂,
(SEQ ID NO: 41)
KKCAWFO(Ac)IT,
(SEQ ID NO: 42)
Ac-KKc(CAWFO)IT-NH₂,
(SEQ ID NO: 43)
c(CNWFO)ITNWLWRR-NH₂,
(SEQ ID NO: 44)
CNWFO(Ac)ITNWLWRR,
(SEQ ID NO: 45)
DKWASLc(CNWFO)ITNWLWRR-NH₂,
(SEQ ID NO: 46)
DKWASLCNWFO(Ac)ITNWLWRR-NH₂,
(SEQ ID NO: 47)
LELDKWASLc(CNVVFO)ITNWLWRR-NH₂,
(SEQ ID NO: 48)
LELDKWASLCNWFO(Ac)ITNWLWRR-NH₂,
(SEQ ID NO: 49)
CWFOITNWLWKK-NH₂,
(SEQ ID NO: 50)
CWFOITNWLWKK-NH₂,
(SEQ ID NO: 51)
WCWFOITNWLWKK-NH₂,
(SEQ ID NO: 52)
CWFOITNWIWKKKK-NH₂,
(SEQ ID NO: 53)
c(CWFO)ITNWLWKKKK-NH₂,
(SEQ ID NO: 54)
WCWFOITNWLWKKKK-NH₂,
(SEQ ID NO: 55)
Wc(CWFO)ITNWLWKKKK-NH₂,
(SEQ ID NO: 56)
NWFEITNKLWGRRRRC,
(SEQ ID NO: 57)
NWFc(EITNK)LWGRRRRC,
(SEQ ID NO: 58)
LWNWFEITNKLWGRRRRC,
(SEQ ID NO: 59)
LWNWFc(EITNK)LWGRRRRC,
(SEQ ID NO: 60)
DKWASLWNWFEITNKLWGRRRRC,
(SEQ ID NO: 61)
DKWASLWNWFc(EITNK)LWGRRRRC,
(SEQ ID NO: 62)
LLELDKWASLWNWFEITNKLWGRRRRC,
(SEQ ID NO: 63)
LLELDKWASLWNWFc(EITNK)LWGRRRRC,
(SEQ ID NO: 64)
NWFEITNWLWGRRRRC,
(SEQ ID NO: 65)
DKWASLKNWFEITNWLWGRRRRC,
(SEQ ID NO: 66)
DKWASLc(KNWFE)ITNWLWGRRRRC,
(SEQ ID NO: 67)
LLELDKWASLKNWFEITNWLWGRRRRC,
(SEQ ID NO: 68)
LLELDKWASLc(KNWFE)ITNWLWGRRRRC,
(SEQ ID NO: 69)
EWFKITNWLWKKKK-NH₂,
(SEQ ID NO: 70)
c(EWFK)ITNWLWKKKK-NH₂,
(SEQ ID NO: 71)
WEWFKITNWLWKKKK-NH₂,
or
(SEQ ID NO: 72)
Wc(EWFK)ITNWLWKKKK-NH₂.

48. A polypeptide having a sequence consisting essentially of

| | |
|---|---|
| DKWX¹X²X³X⁴X⁵WFXITXX⁶XW | (SEQ ID NO: 4) |

wherein X=N, D, S, G, Q, C, T, M, E, K, R, A, P, I, L, V, O, Aib, or other natural or synthetic amino acids, including conservative substitutions thereof,

X¹=A, G, P, I, L, V, Aib, or other natural or synthetic amino acids, or a conservative substitution thereof;

X²=N, Q, C, S, T, M, or other natural or synthetic amino acids, or a conservative substitution thereof;

X³=L, I, V, G, A, P, or other natural or synthetic amino acids, or a conservative substitution thereof,

X⁴=W, H, F, Y, K, C, Aib, or other natural or synthetic amino acids, or a conservative substitution thereof,

X⁵=N, S, T, Q, C, M, E, A, or other natural or synthetic amino acids, or a conservative substitution thereof,

X⁶=any natural or synthetic amino acids;

and wherein the polypeptide has a helical structure.

49. The polypeptide of paragraph 48 wherein X⁶ is W.

50. The polypeptide of paragraph 48, wherein the polypeptide has the sequence consisting essentially of DKWX¹X²X³X⁴X⁵WFXITXWXW (SEQ ID NO: 5).

51. The polypeptide of paragraph 48, wherein Aib may be inserted between any two amino acids of WFXIT (SEQ ID NO: 78).

52. The polypeptide of paragraph 48, wherein WFXIT (SEQ ID NO: 78) is branched.

53. The branched polypeptide of paragraph 48, wherein the branched chain is of sufficient length and/or configuration that the polypeptide binds to Fab 4E10.

54. The polypeptide of paragraph 22, 36, 41, 42, 46, 47 or 48, wherein the polypeptide binds to Fab 4E10.

55. A polypeptide having a sequence which consists essentially of:

XNWFX¹ITX²WLWX (SEQ ID NO: 6)

wherein X comprises 0-8 amino acids consisting essentially of K, Aib, Y, I, or other natural or synthetic amino acids, including conservative substitutions thereof;

wherein X¹=D, C, or other natural or synthetic amino acids or a conservative substitution thereof;

wherein X²=O, N, or other natural or synthetic amino acids or a conservative substitution thereof;

wherein the polypeptide has a helical structure, and is not otherwise disclosed in the art.

56. The polypeptide of paragraph 55, wherein Aib may be inserted between any two amino acids of WFX¹IT (SEQ ID NO: 79).

57. The polypeptide of paragraph 55, wherein WFX¹IT (SEQ ID NO: 79) is branched.

58. The branched polypeptide of paragraph 57, wherein the branched chain is of sufficient length and/or configuration that the polypeptide binds to Fab 4E10.

59, The polypeptide of paragraph 55, wherein the polypeptide binds to Fab 4E10.

60. The polypeptide of paragraph 55, wherein the polypeptide comprises or consists essentially of:

(SEQ ID NO: 7)
NWFCITOWLWKKKK-NH₂;
(SEQ ID NO: 8)
NWFDITNWLWYIKKKK-NH₂;
(SEQ ID NO: 9)
NWFDITNWLWK-Aib-K-Aib-K-NH₂;
(SEQ ID NO: 10)
KK-Aib-NWFDITNWLWK-Aib-K-Aib-K-NH₂;
(SEQ ID NO: 11)
NWFDITYNWLWYIK-Aib-K-Aib-KK-NH₂;
or
(SEQ ID NO: 12)
NWFCITOWLWKKKK-NH₂.

61. A polypeptide having a sequence consisting essentially of:

| | |
|---|---|
| NWFX¹ITX²WLWX | (SEQ ID NO: 13) |

wherein X comprises 0 to 8 amino acids consisting essentially of K, Aib, Y, I, or other natural or synthetic amino acids, including conservative substitutions thereof;

wherein X¹=D, C, or other natural or synthetic amino acids or a conservative substitution thereof;

wherein X²=O, N, or other natural or synthetic amino acids or a conservative substitution thereof;

wherein the polypeptide has a helical structure, and is not otherwise disclosed in the art.

62. The polypeptide of paragraph 61, wherein Aib may be inserted between any two amino acids of WFX¹IT (SEQ ID NO: 79).

63. The polypeptide of paragraph 61, wherein WFX¹IT (SEQ ID NO: 79) is branched.

64. The branched polypeptide of paragraph 63, wherein the branched chain is of sufficient length and/or configuration that the polypeptide binds to Fab 4E10.

65. The polypeptide of paragraph 61, wherein the polypeptide binds to Fab 4E10.

66. A polypeptide having a sequence consisting essentially of:

WFX(I/L)(T/S)XX(L/I)W

wherein X does not play a major role in Fab 4E10 binding and

wherein the polypeptide has a helical structure, and is not otherwise disclosed in the art.

67. The polypeptide of claim 66, wherein X introduces constraints.

68. The polypeptide of claim 67, wherein X is Aib.

69. The polypeptide of claim 66, wherein the polypeptide binds to Fab 4E10.

70. A diagnostic/pharmaceutical/immunogenic/immunological/vaccine composition containing a polypeptide of any one of paragraphs 55 to 69.

71. A method for making a composition comprising a polypeptide of paragraph 55 to 69 wherein the method comprises admixing such polypeptide with a pharmaceutically suitable or acceptable vehicle or carrier or diluent, optionally including or being an adjuvant.

72. A method for using a composition according to paragraph 70, wherein the composition is administered to an animal that generates antibodies to the composition, wherein the antibodies generated are anti-HIV antibodies that may be diagnostically useful or wherein administration of the composition elicits an immunogenic or immunological or vaccine response; or, where the composition is used to detect the presence of anti-HIV antibodies in a sample.

73. A method of eliciting anti-HIV antibodies comprising administering to an animal capable of eliciting antibodies a composition of paragraph 70.

74. A method for detecting anti-HIV antibodies comprising contacting a sample suspected of having such antibodies with a composition of paragraph 70, and detecting binding of the antibody to the composition.

75. The method of paragraph-74, wherein the animal is a human and the method is for treatment or prevention of HIV.

76. The method of paragraph 74, wherein the method is for generating antibodies for diagnostic purposes.

77. A diagnostic composition containing a polypeptide of any one of paragraphs 55 to 69, or an antibody elicited by administration of the polypeptide.

78. A composition for prevention or treatment of HIV, comprising a polypeptide of any one of paragraphs 55 to 69, or an antibody elicited by administration of the polypeptide.

Having thus described in detail preferred embodiments of the present invention, it is to be understood that the invention defined by the appended claims is not to be limited by particular details set forth in the above description as many apparent variations thereof are possible without departing from the spirit or scope thereof.

What is claimed is:

1. A polypeptide having a sequence which consists essentially of:
XNWFX¹ITX²WLWX (SEQ ID NO: 6)
wherein X comprises 0-8 amino acids consisting essentially of K, Aib, Y, I, or other natural or synthetic amino acids, including conservative substitutions thereof;
wherein X¹=D, C, or other natural or synthetic amino acids or a conservative substitution thereof;
wherein X²=0, N, or other natural or synthetic amino acids or a conservative substitution thereof;
wherein the polypeptide has a helical structure, and is not otherwise disclosed in the art.

2. The polypeptide of claim 1, wherein Aib may be inserted between any two amino acids of WFX¹IT (SEQ ID NO: 79).

3. The polypeptide of claim 1, wherein WFX¹IT (SEQ ID NO: 79) is branched.

4. The branched polypeptide of claim 3, wherein the branched chain is of sufficient length and/or configuration that the polypeptide binds to Fab 4E10.

5. The polypeptide of claim 1, wherein the polypeptide binds to Fab 4E10.

6. The polypeptide of claim 1, wherein the polypeptide comprises or consists essentially of:
(SEQ ID NO: 7)
NWFCITOWLWKKKK-NH₂;
(SEQ ID NO: 8)
NWFDITNWLWYIKKKK-NH₂;
(SEQ ID NO: 9)
NWFDITNWLWK-Aib-K-Aib-K-NH₂;
(SEQ ID NO: 10)
KK-Aib-NWFDITNWLWK-Aib-K-Aib-K-NH₂;
(SEQ ID NO: 11)
NWFDITYNWLWYIK-Aib-K-Aib-KK-NH₂;
or
(SEQ ID NO: 12)
NWFCITOWLWKKKK-NH₂.

7. A polypeptide having a sequence consisting essentially of:

| | |
|---|---|
| NWFX¹ITX²WLWX | (SEQ ID NO: 13) |

wherein X comprises 0 to 8 amino acids consisting essentially of K, Aib, Y, I, or other natural or synthetic amino acids, including conservative substitutions thereof;
wherein X¹=D, C, or other natural or synthetic amino acids or a conservative substitution thereof;
wherein X²=O, N, or other natural or synthetic amino acids or a conservative substitution thereof;
wherein the polypeptide has a helical structure, and is not otherwise disclosed in the art.

8. The polypeptide of claim 7, wherein Aib may be inserted between any two amino acids of WFX¹IT (SEQ ID NO: 79).

9. The polypeptide of claim 7, wherein WFX¹IT (SEQ ID NO: 79) is branched.

10. The branched polypeptide of claim 9, wherein the branched chain is of sufficient length and/or configuration that the polypeptide binds to Fab 4E10.

11. The polypeptide of claim 7, wherein the polypeptide binds to Fab 4E10.

12. A polypeptide having a sequence consisting essentially of:
WFX(I/L)(T/S)XX(L/I)W
wherein X does not play a major role in Fab 4E10 binding and
wherein the polypeptide has a helical structure, and is not otherwise disclosed in the art.

13. The polypeptide of claim 12, wherein X introduces constraints.

14. The polypeptide of claim 13, wherein X is Aib.

15. The polypeptide of claim 12, wherein the polypeptide binds to Fab 4E10.
